SEQUENCE LISTING (233 sequences)

SEQ ID NO: 1 - DNA, 693 bp, Saccharomyces cerevisiae
atgcgtaagccttctattacgataacaacagcaaaagctattataactccagactatactcttattaaatcgcattctaaataccaacttccttcacgttttcaaaagctagatgcagactcacctgagagaagtacagttgttaagctattttaccgccgatttatgaggcttaagcctttcatatcgaatgtgaaaatggttaaagatacttacagggattatgttcgatataagtttatgaaagaaaactacgaactcaaacgatacttggtattcaatcctgatggcttgaggtcgaaaatcaatcttgagcttctcagcaacacgaaatgttgtgaaaggatactccccgtgactgagatgcaaagaaccttggaatttgttctgaagagctgttcatacttgcccgaaacaaaagttcaaaagtgggatattgcaagggacaacacatattgcaggcaaattttaaagaaccttttaacaatgcaatatgaaaagtacagatcgattttgcatagaggaataggccatgatgagcttgacgtcaaattctctcatttgaaaactacttctagtccgctaactaagctaaataaaaccgaaaagaaaaagattccgcttttcaaagtatttagtgatttcgatacaacattgatatatctcaatgagacattaggtacaaggttataa

SEQ ID NO: 2 - DNA, 306 bp, Saccharomyces cerevisiae
atgatttttaaacgtgcagtatctacattgattcctccaaaagttgtgtcttccaagaatataggttcggcaccaaatgccaagcgcattgctaatgttgttcacttttataagtctttgcctcaaggaccagcaccagccatcaaggctaacactagattggccagatacaaagccaagtactttgatggggataatgctagtggtaaaccattgtggcattttgctctaggtataattgcctttggctattccatggaatattattttcatttgagacatcataaaggtgcggaagagcattga

SEQ ID NO: 3 - DNA, 402 bp, Saccharomyces cerevisiae
atgagtgagacctgtagcagcagccttgcccttttacacaaaatcctacatatccattcacatacgccttctgtatactacaatatatgcatatcagttagaattctgacatctgaacgtcttcagtgcttttttttctctttctttccagatccaaatatcaccggcagcggcctcaaagtacccggttttcttttttttcacactttttttttttccaaatcatgttgtcaagcattgattgactcattttcctctgactactaccagttcaaaatgttagagaaaaatagaaaagcagaaaaaataaataaaagaaccatatttatttgttcattcactttcgagtataaaattaaatcttgtttttcttgttttcacttaagtacacatacaaattaa

SEQ ID NO: 4 - DNA, 2286 bp, Saccharomyces cerevisiae
atgatgatgtggcaacgatatgcaaggggtgcgccacgctcattgacatcactctcatttggtaaagctagccgcatatcaacagtgaagccagtgcttcgctcgcgcatgccagttcaccagcgtttgcagacgttatctggtctggcaaccagaaacacaatacaccgttctacccaaatacgttccttccatatctcatggacaaggttaaatgaaaataggccaaacaaagagggtgagggcaagaacaatggtaataaagataataatagcaataaagaagatggcaaagacaaaagaaatgagtttggttcattatcagaatacttcagatctaaggaatttgctaatacgatgtttttgaccatcggattcacaattatatttactttgctcacaccttccagtaacaactcaggagacgactctaaccgcgtcttgactttccaggatttcaaaacaaaatacctggagaagggtcttgtgtccaagatttacgtcgttaataagtttctcgtagaggccgaattagtcaatacaaagcaagttgtatcgttcaccattggttcagtagatatttttgaggaacagatggaccagatccaggacctcttgaacattcctcctcgggatcgtatccccatcaaatacattgagagatcttctcccttcacatttttgttccccttcctgcccaccatcattctgcttggtggcctttacttcataaccagaaaaataaatagttcaccaccaaatgccaatggtggtggtggaggaggcctcggcggcatgtttaatgttggaaaatccagagcaaagctcttcaataaggaaacagacattaaaatttcatttaaaaatgttgccggttgtgatgaagctaaacaggaaatcatggaatttgttcactttttgaagaacccaggtaagtacactaaattgggtgccaagattccacgaggcgctattctttctggacccccaggtaccggtaagactctcttggccaaggccaccgcaggtgaggcgaacgtgcccttcttgtcagtatcaggttctgagttcgtcgaaatgttcgttggggtgggtgcttcacgtgtaagagacttgtttactcaagcaaggtctatggccccctcgattatctttatagatgaaattgacgctatcggtaaagaaagaggcaaaggcggcgctctcggtggcgctaacgatgaaagagaagctacgctgaatcaattattggtggaaatggacggattcactacttccgaccaagtcgtagtccttgcgggtacaaataggcccgatgtgttggataatgctttgatgagaccgggaaggttcgatagacatatccaaattgattctcctgatgtcaatggtaggcagcaaatatacctggttcacttgaaaagactgaatctggatccccttcttacagatgatatgaataatctttctgggaaattggctacgcttactccaggttttactggtgcagatatcgctaatgcttgtaatgaggctgcattaatcgctgccagacataatgacccatatatcactatccatcactttgagcaagccattgaaagagtcattgcgggactagagaagaaaacaagagtcctttctaaggaagaaaaaaggtcagtggcctatcatgaggcaggacatgccgtttgtggttggtttctaaaatatgcggatccacttctgaaagtaagcatcatcccgcgtggacaaggtgctttaggctatgcccagtacctaccaccggatcaatatttgatatctgaggagcaattcagacatagaatgatcatggctcttggtggccgtgtttctgaggagctacattttccatcggtgactagcggtgctcatgatgatttcaaaaaggttacacagatggcaaatgccatggttacatccctaggaatgtcacccaagattggctacctgtcgtttgatcagaatgatggaaacttcaaagttaacaaacccttcagtaataaaacggcaagaaccattgatttagaagttaaatctatagtagatgatgcacaccgagcttgtacagaattgctaaccaaaaatttggacaaagtcgatcttgtcgcaaaagaattgctacgtaaggaagcaattactagagaagacatgattagattattgggtccaaggccattcaaggaaaggaacgaggcttttgaaaaatatttggatccaaagagcaataccgaaccgcctgaggcacctgcagcaacaaattaa

SEQ ID NO: 5 - DNA, 654 bp, Saccharomyces cerevisiae
atgtctaagatcacctcttctcaagtcagagaacacgtcaaggaattgttgaagtattccaacgaaaccaagaagagaaacttcttggaaaccgtcgaactacaagtcggtttgaagaactatgaccctcaaagagacaagcgtttctctggttctttgaagttgccaaactgtccaagaccaaacatgtccatctgtatcttcggtgatgctttcgatgttgacagagctaagtcttgcggtgttgacgctatgtccgtcgatgacttgaagaagttgaacaagaacaagaagttaatcaagaagttgtctaagaagtacaacgctttcattgcttccgaagttttgatcaagcaagttccaagactattgggtcctcaattgtccaaggctggtaagttcccaaccccagtttctcacaacgatgacttgtacggtaaggtcactgatgtcagatctaccatcaagttccaattgaagaaggtcttgtgtttggctgttgccgttggtaacgttgaaatggaagaagacgttttggttaaccaaatcttgatgtctgttaacttctttgtttctttgttgaagaagaactggcaaaatgttggttccttggttgttaagtcctccatgggtccagctttcagattgtactaa

SEQ ID NO: 6 - DNA, 342 bp, Saccharomyces cerevisiae
atgtacatgattgtagtaaaatacttatacgccttatgctcaagtttttttgattgcatcttgaattttaacgaaactgtttttgggcccagccacagagcctttaacccaaataacattattttcatcgtcgactttcaaaactttaacattttggatagtaacatgctcgtttcccatatgaccaggcatctttcttcctggcagtaccctacctgggtcttggttttgaccataagaaccaccgtgtctatgcataatagatgtaccatgagatgctctcaaacccttgaatccataccttttcatcacaccagtaaaacctttgcctttgctgactga

SEQ ID NO: 7 - DNA, 2064 bp, Saccharomyces cerevisiae
atgactttgaaaccttcaaagagacgtaagggcaggtctcgccattccaggaagaagcaaataacgtcagcgatactgactgaggaaggaataatgatcaaggcaaaaccatcaagtccttacacatacgcaaatagaatggcagataaacgaagtcgcagcagcattgacaacatcagtagaactagctttcaaagcaacatcagtagaactagctttcaaagcaacagtgataacaacagtatatttgaaacggcttcactaattagctgtgttacctgtttaagcgatactgatacaatagacagatcggaaacatcgacaacggatacaagtaaagatgatctttctgctaatccaaaacttcattatccttcggtgaatggtcaattgccagcaaacaccgttatcccctatggacgaattctggatgccagatacattgaaaaggaacctctgcattattatgatgccaattcatcacccagttcacctttaagcagctcaatgagtaacattagtgaaaagtgtgatcttgatgaattagagtcttcccaaaaaaaagaaaggaagggcaactcgctatcgcgaggaagtaacagtagtagtagcctcctgacttccagatctccttttacgaaactagtagaggttatatttgctaggccaagacggcatgacgttgtacccaaaagggtttcattgtatattgactataaaccccattcatcttctcacttaaaagaagaagatgacttggttgaggagattttaaagagaagctacaaaaacactagaaggaacaaatccatatttgtgatcattaatccgtttggtggtaaaggtaaggcgaaaaaactgtttatgacaaaggcaaagccgttactattagcaagtcggtgttccatagaagtggtttatacaaaataccctggtcatgctatagagatcgcgcgggaaatggatattgacaaatatgacactattgcttgcgcttcgggagatggcattcctcatgaggtgatcaatgggttataccaaaggcctgatcatgtcaaagcattcaacaatatcgccattacagaaattccatgcggatcaggtaacgcaatgagcgtatcctgccactggacaaacaatccttcgtactcaactttatgcttaattaaatcgatagagactagaattgatttgatgtgttgttcgcagccttcttatgcaagagagcacccaaagttatcatttttaagtcaaacatatggtctcattgcagaaactgacataaacactgaatttattagatggatgggacctgcaaggtttgaattgggtgtagccttcaatatcatacaaaaaaaaaaatatccttgtgagatatatgtaaagtatgctgccaaatcaaaaaacgagttaaaaaatcattacctggaacacaaaaataaagggtcgttagaattccagcatattactatgaacaaagataacgaggattgtgataattacaattacgaaaatgaatacgaaaccgaaaacgaagatgaagatgaagatgcggatgcggatgacgaagactcccacttgatatctcgtgatctggcagattctagtgctgatcaaattaaagaggaagatttcaaaataaaatatccattagatgaaggtatccctagtgactgggaaagattggatcctaatatttcgaacaacctaggtatcttctatacgggtaaaatgccatatgtggctgctgacactaaattctttccggcagcgcttccttcagatggtacaatggatatggttatcaccgatgcaagaacctcgttgacgaggatggcaccaatcctgctgggactagataagggttcccatgttttacaaccggaagtcttacactctaaaattttggcatacaagataataccaaagctagggaacggcttgttctctgtcgatggcgagaaatttcctctagagccccttcaagtcgaaattatgccacgcttatgcaagacgttactgagaaatggccgttatgtggacacagatttcgattctatgtga

SEQ ID NO: 8 - DNA, 1041 bp, Saccharomyces cerevisiae
atgtcaggtgtaagctctgttatgctcggtcttcgacctgctacaagaatttttttccgcagtaatatttcggtttcaccttcgaggacttttgtatcatatattggaagatcccagagcacgtcgatactcaaaaatgctcccaacttagaggacaatgtcacaaatcttcagaaaattataccgaaacggttcttttctcaaacatcaattttgaaatcaaggtggaagcctatattcaatgaagaaactactaatcgatacgtacgtttgaacaggtttcagcagtaccagcagcagagaagcggcggcaatcctctgggctctatgactattttggggctctctttaatggcaggaatatattttggctccccttatttgttcgagcacgttccaccctttacgtattttaagacgcatccaaagaatctggtatacgcgttattaggcatcaatgttgccgtatttggactatggcagctacccaaatgctggaggtttctacagaagtacatgttgctgcaaaaggattacgtaactagcaaaatttcgataatcggaagtgcgttttcacatcaagaattctggcacttaggtatgaacatgctagcgttgtggtcctttggtacttcactcgcaacaatgttgggagcatccaattttttctccttatatatgaatagtgccattgcaggttctttgttttcgttatggtatccaaaactggcacgcctagccattgtcggacctagcttgggtgccagtggagcgctatttggggttttaggatgtttttcatatctattcccacacgctaaaattttgctgtttgttttcccagtcccaggtggggcttgggtagcattcttggcttcagtggcatggaatgcagctggttgtgctttaagatgggggtcatttgattacgctgcgcatttaggtggctctatgatgggggtcttgtacggatggtatataagtaaagctgtagagaaacaaaggcagcgtcgccttcaggctgctggtaggtggttttaa

SEQ ID NO: 9 - DNA, 1035 bp, Saccharomyces cerevisiae
atgacattggcagagttgcttggacggtcgagaatagctcaagtagctaacaatcataaaccattgacatacactggcaaaaaatttcatccaacacatcaaatcatcgaaactaagccatcgacactatacagacaagaatgggggttgaaatccgctattccttcgaaaattaaatctcgatatttggtgtacaacgatttggatacgttggaaagaattaccacattcgaacctagaggcggtacgcaatggaataggctaagatttcaagaaatgggagtaccaattgtgtctaatataggcagacaaaatccatttttcaagtacatatctcgtcctgaggatgagtctcatgctaaattgtccttattcaaagaaatgaaaggagatacagatatttcgcctgctgctatgaagaagcgtttgaagaagataacagcactaattagatcgtttcaagatgaatttaaagagtggctggtggaaaatcatccggatgaattgaaattgaattctaataagctggaagactatgtagtgaagtttctaaacaaaaagttggaaacaaaaacgaataaaaaatttaacactgaaatcattggtacaggtggattatcatacagcttgccaggaaagctaaaaaactcaccaaatggtgtcattcaaaggactgtagtacccggtagaatcttgaacgttgttaaggagaacaacgacaataaatggctggcagctattggtgggtttgtggccgatgtggtatttttccaatcaccacctagttcctttaactccatgggagatttcatcagaatgaaaacgttcttgttcgagatcttggaggcatctatggagaaaaatggttctgtttcgatgcacgctagattactcgaaccacaaaatgacaagaccagagaatttttcaataagagaccaatttataaaccattgacctctagaagggcacgtcgaccatcagtgggaaacattcaagaggccaataaccttttgaatataatcaagggaaattga

SEQ ID NO: 10 - DNA, 1572 bp, Saccharomyces cerevisiae
atgaaacatccgtatgaggaattccccacaggttccaagtcaccatataacatgtctagaggagctcatccaggcgccgttttgctatcaccacaatcttctgccataaacaagaacaatccgggcagcaattctggcaataaccagggcaattcatccgtgaccgcgaatgttttgtcccctcaatcccatagtatgtcactcaatgacatgcttgatcagcaatcctttatgctagacactgcgggaaccagggctcagccgctccagcaacaacagcagcagcaacaacagcaacagcaggcgtcgttgccttcccttaatattcaaacagtttcgtctacagcagctggttctgctatcgtttcccccatgatgcaatctccaaaggcgttgcaatctactttatcttcaacatctatgtatttggattcatttcagagatctccaaataatattttgggtattccatcgcagagtggttcaattcctttgccgcaatcccgtcaatcgcaacaacagtctcaatcccaaaaaaacgatccgaacatgggcaccaatttcagccaagacatcaaccaattatgttcttggatatccatgctgaacagcagccaacaaaatactgtaatggacaatatcctttccattttgaatgatgatgttttgaaatatactaagttaaaaatagagactttgacaaacactcccttcatctccccaccgctacctgcaattgcatcacctatacctaatagggatgacacacaaattttgaatattgattcagtcttttcttcaagtcctataactaatgaccctgaaaataccgataacttgttgtatcaaaattggtcccctcagccacattcaataccaattagccaacccatatatgacaacatcaccgacgctagtcaaaggtccaagtcggctgagcctcatgttaattcaagcccaaatctcattcctgtccagaaacaattcaacaacggtaacagtaccaaatataaaaagttgccatcagagaatcccaactatctttctcactctctctcaagttcccattcttttttccaaccaaagaaaaggtctaacatgggaaatgagtacaattctcatcaccatcattcattgcatcatcctctgcataataccacctcatatttttcgaatacttcaagaccatcaggaactgatttgaataagtccaaccagaacgtgttcaataataccattacgcatcctaatgctggtcctacaagtgccacatcgacatctacatcatctaacggaaacactcctttatcgagtaactcttcgatgaatccaaagagtttgacagaccccaagctactaaaaaatatacccatgtggttaaaatcgttaagactacacaagtattcagatgccttaagcggaaccccgtggatcgaattgatttacttagatgacgagactttagaaaaaaaaggcgttcttgccttgggtgcaaggagaaaattgttgaaggcttttggaatcgttattgattacaaagaacgtgatttaattgatagatctgcttattaa

SEQ ID NO: 11 - DNA, 489 bp, Saccharomyces cerevisiae
atgtcttcttcccaggtcgtcagggattctgccaaaaaattagttaatttactggaaaaatatccaaaggatcgtatacaccacttggtctcattcagggatgtacaaatagcaagatttagacgtgtagcgggtctgccaaatgtagatgacaaaggaaaatctataaaagagaaaaaaccctcattagatgaaataaaaagtataattaacagaacttccggtccattaggactgaataaggagatgttaaccaaaattcaaaataaaatggtagatgagaaattcacggaagaaagcatcaacgagcaaattcgtgccttgagcactataatgaataataaattcagaaactattacgatattggcgataagctctataaacctgcaggaaatccccaatattatcaacggttaataaatgccgttgacggtaagaaaaaggaaagcttatttactgcaatgagaactgtattatttggtaaataa

SEQ ID NO: 12 - DNA, 852 bp, Saccharomyces cerevisiae
atgtctgaatcatctatttcttcttctaaaccatctgtggaattgccacaagcaacctggtcgcatctgcaaagatacccagctttatccaagtttattaaatatgcggaatctctgccacctgtggagagattgatttccttcaacctcgttgttttgggatctgtgaaccagtgggtttccgaatcgtccagctctcctcgtctggtgaagcaagttgttgctgctgggaaggaaggggccttcaagttggacgagttagttaacctcttggtgttcaaggagggtgtcgacggcttgctgtacaattggaaatcacattccaacacgccagggatctggctggtgtggttcttcgtcgactacgtcgccaacatttctaatactctgttgagggagttcctgatcaagccattgcacttgcaaggttctaccgcatcgaaggagatcggctcttccggtgaggagaacaaggtcactgatgcttcttctttgccccacgtggcagagttgtcttcaacgaccagaggtatgtcgcaggagatccagtccaaggtcaagtcgaactatatcgacccaaccaaggacctggctaaagaaaagtacgacgccatagtgaagcccacaactgacaagttgcagtctgtgtacatcgacccaacaaagactaagcttaacgaaacctaccaacgcttcaccactgtctatgaaaacaatctaagtaaatctgaaagcgtccctaaagccattgtatccaccgggttggacttgggcaatgccaccattgagaagctaaaggcctcaagagaagaccaaaccaattctaagcccgcggctgtgtcgaccaattag

SEQ ID NO: 13 - DNA, 1479 bp, Saccharomyces cerevisiae
atggctaatgatcaagatcccaacaagtctctcattaatgacgctttgactcggagtatgtccgaattttatgatgatgatgatgataacgacagtgacatgtgtagggcaaatgatgagggagaagacgtttttgaccttccacttaaggtaggcgtttctcaaagcaggaacttttcagaagtaaatgacgtgttagacccgctttctagtttgcatggtccgtccaagaaggtgagatttgaacagcaaaagcagcaacagcaacatcagcaacttcacaatgacttcaatacagatttcaacctgaaaagcccgtccagtaaaaaaatgggggtagaacagctgatccagtcagccaatgaaatcaacgattacctcgctaacaacattgacaaggtaaatagctttaactcagagctattgagcggcagtggcaagttacctgggagagtaaagagtgacacagcaactcaaggtaccggacgcttagattccatgtccaactttgctcttagcgatacggagttggacaatgatgacgataattatctcctggatccccttgcaaatgctagctctactacacccactgtggaacaccacggttacagcttattagacaaggctttatccacgtctgataaggaaaaaatatatacgaataaagtgaattcgaatagtcagatagatactgataatcactctcacgaaagtggaaacactaccaataacgaaacagatgaaaatgagagttcggagatcctggactacacaaaatttgattctttcccctatcccccgtcttctgcccccaatggggaaccaccagatttgaaagtgctaagcattgaatgtgagcaggaaaatgaaaaagagctacgacgaatatctttattattggatcattacgaatcaataccgaaaataccagaattgtcagacgatgaagcactatctaagttccgtgagaatatcgaattaattttacaactatctaagaaaattaacgataatgcgaacactttagcgatttcctccgaagatcctcaaaaatttgttaattttgtgatgaagaacccaccttccttatcattcagagattttatcgacagaatccagaataaatgcatgtttggtgccgtagtctatcttggagccacctatctacttcaacttgtgtttcttacgagagacgaaatggacggtcccataaagttgaaggctaaattacaggaggaccaagcccataggatcatcatatctacaataagaattgccaccaagctattagaagactttgtccactcacaaaactacatttgcaaagtcttcggtatttcaaaaagactgctgaccaaactggaaatttctttcatggcgagtgttaatttcgatggattaatgattacttgtgaaaaattagaaaaaacattgcatatccttgatgatacaagacaggcattaggaaacacatga

SEQ ID NO: 14 - DNA, 1296 bp, Saccharomyces cerevisiae
atgaccccacctcatttttttttatctctcataaaaaaaagatgcatatgctggatatgtttggaagaatccacatatgactctacatggctacagcacacgtgcggttgcaacttacaaattcataaacgctgctatataagatggctctatcaaatgcatgttgaactttttcttccgaacacagtggacctaccaaaggatgctgatttacctataatcacctgtttaaaatgtctcgttgatgggcaccatgattttatgacgactttctctcttacagaaatatgggaaactcgtcccatttgggggcaaaaatcagtaccgtttcaaaatgactacgtgttcaatctaatgtctctttataccaaacgggataatcatccaccttatgtgttagtgaaatttggcgaatgtcctcaatgcaagaaaacaaatttcataaaaagacctacagtaaccatccagtcttctgtactatccctgttttatcagtggcaaaaaattacacgatacgtgataccattagggataactagtttatttcttttaaaccctgaaaagacaagttttgacattggattatggcaactacgctgccttttccctgagaatgttctacgaaatatgttaaacatcagcactacaaaggcattggacgtatatgcgcaaactgaacgtggcttattaagcattcctttaacctcctcgatcataatatatgggttcatacattatcttagcaacatttctaacgtcagtgctaatgcaatactctttaagtgggtgtatctcagtattgtaaaaactgctggaaacaaatattacaagggtataggtttacctaaaataattttgtattctaacttggctacattttgctataattttacctttaaaagattggttgacttaatttacaggaggctgatcaataaaggaggtaagtatttgtatcacggaaattttgaaaactctagtaatagcgttccagctgaggaattttttatcaggcggaattggtatgctattcttgcagaaaagatactatggccctttgttggtaaatgtacaggaggtcttctattgaatgcttttctatggatacagagaaagtttaaaattgaatggacgccgaattgctccccttcggaatttagaatgattttcaatattataggttgtggtactgctgctataggctggtcaagtctgaagctctatgccagttacaaaagatgtcaggaacttgaaaagatcaatgaatttattgaacaaagttgcaaaggtgagtaa

SEQ ID NO: 15 - DNA, 2409 bp, Saccharomyces cerevisiae
atgagatttcttcaggtttataaatcatcggcattaattggactaataatattgttagccagtaaagtaaatcttgcagaggcaaaaaggaagctggtagccacatcattggtcacatgtatggaaaactcgcagctgtcagcaaatagttttgacgttgttttcaatccagacgacaggtcattacattatgatcttgatatgtcaactcaaattgattcatatatttttgctgatattgatgtctacgcatatggtttcaagatcattactaaaaatgtcgatctttgttcaatcaactggaaacagttttgcccggttcacccaggtaatattcagatcgactctattgaatacatctccagtgagtacgtaaatgaaattcctggtattgcataccaagtccccgatatcgacgcatatgccagggtcaagatcaccaacaatgtgagcgagtacctagcatgtatccaaatatacttctcaaacggtaaaacagtttctcagattggtgtaaaatgggcaactgcagtggtagcgggaattggtcttttactatctgccatcttatccacttttgggaactctaccgcagcctcacatatttctgccaacacaatgtcacttttcttatacttccaatctgttgtcgtggttgccatgcaacatgttcatcgagttccgcctattgcagccgcctgggcggaaaacttggtttggtcaatggggttaattagaatctcattcatgcaaaggattttccggtggtatgtgcaatccactggtggtacgcctagcttatatctgacttcaacatctatgtctgttttggctcaaagaagttggcaatatttgatggagctcccattaatcaaaagagctaccaatgttttgtatggtaatgcaaatacattaatttttagaggaatcaaaaggctaggctataaaatgggaatcgaaaatacgtccatcgtttgtaccggttttactttcttcgtcttatgtggatatgtgttggctggatttatcatagtattcaaatgctgtgtggagttagccacccggttaggatggatccaaaaggcaaggttttgggaattcaggaaacaatggagaatgatcttaaaaggtgctcttttaagatatatttacatcggtttcgttcagttaacaattctcagtttttgggaatttactgaaagagattcacctgctgttatcgtcatcgcatgcctttttattcttttgtcatgtgggttaatgctttgggctgcatggaggactgtttttttcgcaagaagatctgttgcattgtacaacaacccagcagccttattgtatggagatgaatatgtcttacacaaatacggatttttctatacaatgttcaatgccaatcattactggtggaacattgttcttttatcctacatttttgtaaaatcattattggttggatttgctcaggcttctggtcaaacacaagttcttttcatgttcatattggatttgttttacttcgttgctattatctactataagccctatctggaccgtccaacaaacataatgaatattttaattgctactgttactgtagttaattcctttctattcatgtttttttcggacttgtttaaccaaagctacaaagtggctgctattatgggatggatatttttcattatgaacgctgcattttcgttcattttactaatgatgatattggcattcgctggtatgatgcttttctcaaagaaccccgatctaagatttaaacctgctaaggacgatagaacttctttccaaaggaacactatgaaaccagaaggtactgttaatagatcagtagcgaacgagttgcttgcgttgggcaatgtggcaaaagaccatgacgacaattcagattacgaatcaaatgatactggtgtgaatgatgagctaaagcaagcacaggatgaaaccactcccacaaccgtaacatcaagtgatgataacaagcctacgttttcagagaaaatattaagtaaattctctaggcctaagaatgaaaacgcaagcacagatgctctgagagttgaagctccaaaacaacaaacgtttcctcacaacttaacgaatctttcacgagaaaacctatcaacattaggctctaaaccttatccaggacacacaagatcacagtctgatgcgcataatggactgatcaattcctttgaagaggaagatacaagttcaaataccgatccatttcacgactccactgaaggagaccttttagatacctcaagttcggacggaggatttaggtcgcaaaactatgttcgagatgacagcataaatagtttgggaaacaataaacaaccactgaggaaaccccctggattcttcgatgaaggatttatgtga

SEQ ID NO: 16 - DNA, 831 bp, Saccharomyces cerevisiae
atgctttttaaacaatggaatgacttaccagaaccaaaacatctgctagatctcccagagatttcaaaaaacctgcaatctttagaagtctgcccggttccaaaagttgagtttcctcaggacttggatgttcctcaatattcgacagccgtcattactactaagataatgaacccattattccccaaaaatctgcttcaattaacctctattggtgaaattaaaactacattgacggtgaaaagtccaagtcttccccaatcttcaggtaaacactcgtggaattacgacgaaaactttccaaacgaggttgatcctgatcaaaaaaatgacaccgccgatgaaacagtatatggtttttctttcccgatttattcatttggaaagacgttattattttctatggaggagaatttcattagtatatcgccgatttttggtaacatgataagtagatctatcatatcacagttagcccagttctcccctgatataatagtaattggcacctctgataaaatcgccagcatgaaggtaatgacggaaaatgaatgtacactacaaccaccagaatttataacgggattcataggaagtgtgctaacacaattgattgtcggtcccagtaagggactgaaatttaaatgtcttgtagcgcctagtgaggggcctaatggatttgagaaactttcgttgtctgacatgggttctttagttgatttgtgcggtcaatggcttggttttgagccttcaagatattctgaagaatgctatcgcttatggaggtgtgatagcgcggcaattggtgcacaatcaggcctatatatatga

SEQ ID NO: 17 - DNA, 1707 bp, Saccharomyces cerevisiae
atgtcttcattatatactaagttggttaaaggggcgaccaaaataaagatggcccctccaaagcaaaaatatgtggaccctattctctcgggcacttcgagcgcaagaggattgcaagagattacccacgcattggatataaggttgtctgatacagcctggaccattgtttataaggcattgattgtgttgcatctgatgatacaacaaggcgaaaaagatgtcacgctaaggcactattcccataatttggatgttttccaattgagaaagatttcgcatactactaagtggtcttccaatgatatgagggctttgcagagatacgacgaatacctaaaaacacgttgtgaagagtatggcaggctaggtatggatcacctacgagacaattactcctcgctaaagctgggcagtaagaaccagctttcaatggatgaagagctagatcatgttgaatcattggaaattcaaattaatgccttgataaggaataaatactctgtttcagatttagaaaaccatttgttattgtatgcattccaattgttagtacaagatttactaggtctgtacaatgctcttaatgaaggtgtcattacacttttagaatcattcttcgaattgtccattgagcacgcaaaaagaacactggatctctataaggatttcgtggatatgacagaatatgttgttagatacctaaaaataggaaaagctgtcggtttgaaaatccccgttatcaaacatattactacaaagttaattaactcccttgaggagcatttgagagaggaaacaaaaagacaaagaggtgaaccttctgaaccgcagcaagatagaaaaccatctacggccattagctctaccagtagtcacaataacaacagcaatgacaagaataagtctatagcgcagaagaaactggaacagataagggaacaaaaaagactgctggagcaacaattacaaaaccaacagttgctcatttcccccacggtcccacaagatgcttacaacccatttggatcacaacagcaagatttaaataatgacaccttttcattcgaaccaacccagccacaaatgaccgctcaagtccctcaaccaactgctaaccccttcttgataccgcaacaacagcaacaggctttacagttgacatctgcgtctacaatgccacaaccaagtgagatacagattactccaaatttgaataatcaacaaactgggatgtatgcttctaaccttcaatacaccccaaatttcaccggctcagggttcggcggatacacgaccaccgagaacaatgcaattatgaccggcactttagatccaacaaaaacggggtctaacaacccattttcgctagaaaatattgccagagaacagcaacaacaaaatttccaaaactccccaaatccttttaccctccaacaagcacaaaccaccccgattcttgctcatagtcaaacaggtaatccatttcaggctcaaaacgtggtgacatcaccaatgggcacatacatgacgaatcccgtggccggtcaacttcagtatgcctctactggtgcgcagcagcaaccacaaatgatgcagggtcagcaaacagggtatgttatggttcccactgcgtttgtccccatcaatcaacaacagcaacaacagcagcatcagcaagaaaatcctaacttaatagatatatag

SEQ ID NO: 18 - DNA, 1875 bp, Saccharomyces cerevisiae
atgttatcaaggtacaaccgcgttattgaaataaatgggggtaatgcagatatttctctgcccattgtcaaatttccaccgtttaaactcagagctcaattaattgaaaaggaccccgtagtatggctgcatctaatagaaacctatgtaacgtattttgagtatttgatgcaaggtgctaacgttgaattattagatgaatctacacttgatcatcttcgtttatttttgagaacttacttgcatgaaattgctgatgaggaaggaaagttattaagtctcggtatcaaccatgatgtgtctgaacagttatatttactaaaaggctggatattttcattgatcaagaaatgcgggttattacatcttcagatttttggtgactccctgtggaatttgatcaaagtatatgttagacgtaatccagattctattcgtggtttgatcgatggctcattgaaaccacggatcaacacgcaacgtgtacaattggacaaatcgtaccaagttcaacagcatttgaagcagttaatagaatctggaaaatttaaaagaatagatttaaggtgcgttgaagatctgctgtctgccaaatccatgcagccaaataagtttgcggagaacttctttacggcaaattggattgaaattttagaggcattatgggcaaagggacagggccggggtcataaagaggctagagaattgatcattatcagtttattttctgtttctgctgatcgtttactaaaaataactaaagaattaggcataagcaattttgaaactttggcgttatatcctcttttaggaacaatgctcataaatgaaggagtccacaaaaggcttcctgatttgaagagcaagttactatttctgaatttaggggggttgtcaatggacgaaggagaccatatgagctatccaacatcatcaggtactgaagttaatgaagaacaactgtccgcgttgatggaattgttcccccagttttctaaatatcaattatctcaaacgttgcttgcctatgataataacattgaattagtaacgaataaaatatttgaggatcctaccattatcgaagcattctccagagaacctgcggaggaagaagtagagccggtgtcagatggtgataatgcatcctttactgaagaactgagtatattggatagaggagatagctcaaaaaataaagagctagacaaaaagattatctcggagggtgttccggatgaattaagaaataaaactttgacaagagcattgaaacttttatacgaggctgatgaagatgaaagggatgacacttatgatgaagccgatgtgaaccggtccgatccttcgaaaagaattggacttcaagaagacgaagaaagctatgatacaaaagatgattctaatgaagtgcgacaagatcataactatcatattgttgaagcttacttgtggaatctattgaaagaggatccgaaattatttgagagatcaaaaagaggtacaaaagtcaggaaaactatgaaagaaatgacttcatggtcggatgaaaaaatcgaaggttggtgtcggatgttagaaagatctcctacccgagcaagacttttagaaaagaagtttatgttcaaaggaaatagcaaaacagggaaaacgtcttatgttcataatcgtgatagccaaaatgatggaaatgtcgtcaaagaacaagccaaacaaaaaaaatctgaaaatattaagaaacatgaacctcaatctactgaacagaagaaaagacagcatgctaagaatgaaaaaaggaaaggggctagagctaaccataaccgtaagaaggggcacgataaaaaattggctcgtgcgggaaataacgctatttga

SEQ ID NO: 19 - DNA, 939 bp, Saccharomyces cerevisiae
atggtttcattgactacaatagagcaaagcccagtcaaatgcgagacaaccactgagaaagagagcaatgatactagggggacagattcgaacgagaatgcagagacaaaagaaacaaagaagggttttccattccatgacttggcaaaattgcagaaacagtacaaaaataaatccagtaggaacgagtcactagtagcactaatatatctcttgggttcgatgctgtcattttgtctcctaatatttttcacggacttttatttgatacctttgttccccaccaccactacaatgacagactacattgtattcaatttttatttattgaacgtttttgtattttgtatggtgcatttcatttatcacttcgtcaaaaatatttctctacaacaacatctcgaacattggcagaagttttcgtatttatcgaatatcaacttattgatatcatcacaaatcacaattctttattatctattctatgattacgtgtttttcttcaaaatttttacccttttaatgaacttcataggattagtggcatacttttttatattgacggacaaactcatttcctcgaaaagattcaataaaacagttttcttcatttcggtatcagtagtgtgttgttccttgccactattaaccgcaataatcacctttgatggtttagaaaacctgaaagagaggataaaagtgaacgcaattacgtgggaacttgtggctttggtagcggcatctataatttacgttaccaggttccctgaatcattgttcagacgaaataaaaaggaagaaggttggaatcattccgagtaccttttccatctcttgatttctggtacggctttttaccattttttcattttgattcaatcttatattttgatgcattcaagtttgaatcagcccgaattaattaatttcaagtcttag

SEQ ID NO: 20 - DNA, 888 bp, Saccharomyces cerevisiae
atggctacgttcaacccacaaaacgagatggagaaccaggcacgtgtacaagagtacaaggtctccactggcagaggcggagctggcaacatccataaatctatgtccaagccgtctcccgtacttcttccattgaaatccaattcaaagacagtagcaaacaacaataataacggtagcaaccaggaaaaggttcctcgttttgcaattggtagaggtggtgccggtaatatctttcatgatccgcacctgacgagatccgcccaacaactggactctaacgacaatatcaactataacgacgtgatcaatgacatcgacgattatatttcccctataacttcggatatggtggatgaaggtgggtcaaacccggtgaccaacaccaggtctcgtatcagcgctacaaggagccatcagtctttgcatgccaccacttcgtctccgaacaacaacgccccaattgtcgttggtagaggtggggcaggaaacattttcttcaacaaaaaaaaggtggccagcaatggtggaaatgaagaggatgagatacgaggcggcaatatcgaggatgaggatactatcaatgcaaacgaggacaacctgtttgtggtgacttccaatggcaatgctttggcggcaatcaagtccacgtccaagaagcctaaaaataagctcaagggcaaaagcgcaccggaaaaatttgctattggaagaggtggcgctggaaacataatttctccaaagtcaagcaggaacactatcaaccataacttaaacgatgatgatgaagataagtttaatttgaaagatgacaatggtaaagagaagaagaagaagaagaagaagaaatcagggtttttcagttctttgaaaactatgtttaattga

SEQ ID NO: 21 - DNA, 1461 bp, Saccharomyces cerevisiae
atgcctctatcaaaggtggagcactacctttcataccatacgcgcttactcttaccccatgttttgtctcttcagtcatcacatcgtgttgcatacatcttttcgctattatctgcggtgtcaactggcttcattactttgatatctctttactctcaaccgtggcagaaacatttaaattattcctcatggcaaatcaacaccatcgctagtatgactaatttggggatgtacttgacgccaccaatcttggggatgatcgctgattctcatggccccattactttaagtcttttagccatcatagggttcatacctagctattcatatctggcttacgtttttaatcatccggagttatctctcggaggaaatggtgactcatcattcaatctatccatcatttgtttcgttttcataggtatatcaacaagcgctttatactttagcgctttactgacatgcactaagctatatcctcatacaaaactactatccattagcttaccaacgacatgttatggtatttcttctgtagtcggttctcaactgctaagaatcaaatggttctggtcctctaacgcaagttcttcctcgtccaatagtgacttaaacctgggaagagtattccaaacatttgccctcgtttatgtcgttattgggctacttgcatggatagccaccagcgtggtatcacttttgcattttaatgaagagcaagacaaccaaaaacggctggatgatcaaactgatgtggaacaatcaccgctgttagaacgaagtaatcatgttcaagaaaagtttacgcagacgatgctaaggatctttagtgatcctgtgacatatatcctagcggtatcaattttgttatcacttgggcccctcgagatgtttattgccaatatgggatcactgactaacctgctagtccaattagatgcgccaaccttatctacaaagttgttatccacatacgcgctatcttccacttttacgagattgctcacaggcatagtggcagacttcttcgccaagaaaaaaatatcaattaaatggatcctgttgactttcctttcattaggggtatgtgcacaactgtttttattgaaaatgacctcttcagcgtcaccctgggggctagtacctacaggatcattggttggaattgtatacggtggacttttcactgtttatccgacgctggtcctgttagtatggggcgaacgctcattcgggactgtttacggtagcttactaattgcacctgctataggttctatgatattttgcatgttgtatgccaaattttacgattctcgctgtatgagtggcggaggagatctgcgaaatccgtcctgtatttcggctgtctacaagtacagcagtatcgcattcgttgtatccgctgttctttcagctgtagtattttggaaattaaaaagtagaaaactcagaatttaa

SEQ ID NO: 22 - DNA, 876 bp, Saccharomyces cerevisiae
atggcacccagaaaacgctttaggctactcgaattagggtcaccaacacattctaaaaggaaagttcaaaaacctcttcaagagaagaccccaaacttgcgagtttctccacttgcttttaaaattggtaaagaaatcaagaataaggaaatcagaaaaacaaaaaaaacagaatcagaaaatatattcaattcgaaacatgtcgatttaagactagaaagcccacatccaggattaaattttgtaagtgatgctcaacagtactcaaaggctggagatgttcgatatttgaaaaataaatcaagcaataccttgaaaaacgaaagacaaacaatagagcgcccctcatttgataattccttaaggttcgaggacattgagcagcctccaaaatctacttcaacgccggttctttctcaatcttctcaaataaatgtagaaagagaagcacccatgtttccagttccttactatattgctccttctcctatgtataattttagcccgtatcaaaatttcgttggaaacccaacgttcctgacgccaagtcataacccaaacctaaactatgccattcccatccaacggccggagttgctttatccgaatgttaacgtatacgattcgccattgtttaagaaaacaagactgccccatcagaccaaaagtctagataaagaaaaaaattaccaatatcttccaatatatccggtctcgatttctaataacggagattttgttggccaagagacaccaagggcagcaccaaagttaagtaaaaagagactttcaaatacattggatgttaattgttccgattatgagtctagtggacaaaatgcaacttataatgatagtgagtcttcccttaattaa

SEQ ID NO: 23 - DNA, 1185 bp, Saccharomyces cerevisiae
atgtggacaaacactttcaaatggtgcagcaaaactgagaaggagaccactactgcagacgccaaggtgtgtgctagcgtgcagggcctgaaggccctgcagcagcaaattatggatagcaccactgtgcgtgggtccgttaataacacgatgactccaggcgggattaaccagtggcatttccacaataagcgtgcgaacaaagtgtgcacgcctacagtactaatccatggatacgctgcctcgtcgatggcgttctacaggacgtttgagaacctgtcagacaacattaaagatctatatgccatcgacttgccagccaatggtgcctcagaggcgccagcattgcaagtgaacaaaaccaagaagatcaagtctctcaggtttaagcatatagaagatgacgtagtaatccctgtgatagagaagcgccccccagcagaggacatcaagtcacacctggagcagtacgaaagctacttcgtagacaggatagagcaatggcggaaggataacaagcttcgcaaaataaacgtggtgggccattcattcggaggatatatttcattcaaatacgccctcaagtaccctgattccattgagaaactgtgtctcatatctcctcttggtgtggaaaacagtatacatgctatcacccacaaatgggagcccaacaccacctatccacttacgtttaccgacccatcgtcccgatattatacaagaaagctgaacgtaccgcggtttatctttgaaaaccagctaaacgttttgaagtggatgggacccataggctccaaattgtgctcgaactacatctccaccgcatacgtaaaagttccggatcagatatataaggactatcttttgcactcatttgtcgggaagaaccagacggtgcaaccacagacaatcaaggtgttcacccatttgtttgaaaggaatctgatcgctagagatccgataataaacaacgttcgttttttgaacccagcaacgcctgtcatgttcatgtatggcgagcacgattggatggataaatatgcgggctacttgactactgaatcaatgctaaaaaacaaggcaaaggctagctacgttgaagtccctgatgctggccataatcttttcctggacaacccgcagcactttgcctcttctttagtctcgttcctgtcaaagtaa

SEQ ID NO: 24 - DNA, 528 bp, Saccharomyces cerevisiae
atggtccttttacacaaaagcacacatatatttcctaccgattttgcctctgtttcacgcgccttttttaatagataccccaatccatactccccccatgtactatccatagacacaatatcaaggaacgttgatcaagaaggaaatttgcgcacaacgaggctgttgaaaaagtccggaaagctgcccacatgggtcaaaccctttttaagaggtataacagaaacatggataatcgaagtttccgtagtgaaccccgctaactccacaatgaaaacttacactaggaatctggatcacactggaatcatgaaggttgaagaatatactacctatcaatttgacagtgctacaagtagtacgatagcagacagccgggtgaagttttcaagtggcttcaatatgggtatcaaatctaaggtagaggattggtcgcgcactaaatttgacgaaaacgttaagaaaagcagaatgggcatggcatttgttatccaaaaactcgaagaggcgagaaatcctcagttttga

SEQ ID NO: 25 - DNA, 1335 bp, Saccharomyces cerevisiae
atgaacgtcgttgtgtgctccggtgggactgccacaaattcattgactccttgcttttctaatatttctatcttaaaaggacatgagctgacgtatatattgccaatatcagataatggcgggtccacaagtgaaattctacgaatagttggtggcccagcaattggtgatatcagatctagaatagtgagactacttcaagatgaacagcttgtcgagttgtttggccataggctacctaatgataagttattagctaagaaagaatggaatgaaatagtggaaggttcgcatcccatatggaaaaatatttcgatagaagttaaggaaatgtgtcgttcttttattatccatatgcaagcagaactcttgaaaaaaatcaaacactcgaatccatttcagtttgagagtgcatccattggaaattttttcttaactggtgctagattgtttttaggttctctagacgcatctattgaactaatgatgaggataggaaggtgcagtcctctagttcatgttattccatgtataaataccaatcacacacatcatatctcggctttgttgacgaatggtgagatgataactggccaatcacaaatatcgcatccctcgaaatctgttcctaaagataatagcatagcacattcagctaaatttattcatttacttggctcttatgacgaccatttgaaaattctcctcgacgatgaggaagaagaagcggaagaggagtatgccaatcctatctatattcttccagaactgaaaaactcccagttgcattttgacaagcttgatgaatcacagaatctacctgctccggtacataggatattgtatataaacccatatggcgaagagataaaaccaatgggaaatccaagagctatttccaaggtgaaaaaagctgacatggtcgtttattctatcggttctttaatgacaagtttactcccaatccttattcttggtaatcttgctgaagtaattttggaatctaataatacgaaaaaagttttgctcataaataataaatatgatagagaggtttttggtttagacggtttacactacgttcaaatgatcatagattctatgagtagggcaatagcaggctataggcaatctaagggtgtccattcagagaatgatgactttgaatggcaagattttattactgatattgtttacctaaaaaatggtgaaattgaaattgatgaaacgatattcgagaagcatagtattagatgccaccagatcgcgtcttcggataagatggaaagtgaagaacttgaaaaggttctgaatcaaattggccttaaaaactga

SEQ ID NO: 26 - DNA, 195 bp, Saccharomyces cerevisiae
atgtctagtcgccaaggtggtaagatgaagcctctaaagcaaaagaagaagcaacaacaggacttagatcctgaagacattgccttcaaagagaagcaaaaggccgatgctgctgctaagaaagctttgatggctaatatgaaaagtggtaaacctttagtgggtggtggtatcaaaaaatctggtaagaaataa

SEQ ID NO: 27 - DNA, 2316 bp, Saccharomyces cerevisiae
atgataaaaatgtggaggttacagattgtactggtgccgccaagtgctcaagatattatcacatttctcgaagcacgcttgaatactccacaatcagtgtctcctatggtccagtataatgaagatataattacgcacaacaacagtatcaataactgtagcgacccctctcctacgagcccgtcttcgcaaaattctattcagtcgaatcgtagtagtgatttcattaactacctcccgaattgtaaaaagtttttacattttacggacggtgacaatacccttttacaactatctaatgaaattttaactaaatttgatagactttatccaaatttcaaagaatccattgaaatcgtatctctacaagataggcatggttgtgatttggactctgaatttataataaaagacgtatttgaaaatgatggtgtggttttggtaattttaaaagatgaattagactggagtagaaatcaacatatttcacttttacaattggcaagacagcggagacggcaggacaacaagccatccacaaaatccatcgttactgaaaagagaaaaaagatctctaaggaagacttatcttctatatcaaataaagatacaatgcatctaatagctaaaagctcgctcaagaataacttcattaataaatcaagagtatcaacgccgctaatgaacgaaattctaccgcttgcaagcaagtatgatgcactgaataaagaaaagtgtcccatgccacttacttccacagtagtggcatcaaatgtacacaaagacgtaaaagatcacgctcgtgctaaagagggagttgtaacgcaagggagtgataacaataaagaaaatataccttcatccactcaacaacagaaaaatgatggcgctaaaagggccgaatcaaaggatttagatcttttacgcaattcgtcagaagatgctgattatgaacccgcagatgaaaactcccctcaaatttcgtttgattcgattgataccgacttccaattgagcactacttctcatactaattctgatatgcatatacaatattctaacccctctagtggtgctcatagtcccaggaagagttccttagaaattaaagttcaaaacaaaaaaggtgatgatctgccattgaacgacaaagatataggtgagaattgtagaaggatagaagcattttcagatgaagaggattttaatgaaaccgataatgatcgtgcggattcgtttatcaacaattctaagaaggcatcgatgggatttcgggatatcaattccgacttggatagcgtttcattcaacagtgatattgaaaatgcagtacaaagtactcaaagtaccaaaaatgttgtttctcctcccttttttcctgaaaaagaattgaataatcggttacatcaaagtcaaggtaaggaagccctatttcgacttgttgagaaggagtttcctgataaatcattgggtgctgcgtcatctacttcgcatgcaaaagacgtaaaaatacaagaaactattcggaaactaaataggttcaagccaactggggaaactaaggtccaaaaaaggaatagtattacggaaccatattacggaaaatttggaataatgaagaaagataaacccaaatccataacttcaaaaggggtaagtctggaaacaaagcactttgatgatcccaataccattatttccggtgagaaattcgcaaaatttggtaaaataaaagtgaaacgaaaaactgatgatgttggatcgaaagttatagagtttaaaagaaaaagaaatatggggaatagatccctaaaagatatttttgccaatgctgggaaaccgccaaatgcagcttcgaccataaaagtagtcaaattaatgagagatcctgttgataatagtaaagacaaagtggaggcaacttctaatagtacggctcaggaacaagaacaagtctcacccaagttgccagttatgaattcaactcctggaaaaagaaaaaatggccaagcgataccgtccagtttagaaagaacaccgcagctcaagaaagtcaaggttaccaggtcgcattcttcaccttcttcttcttcttccatgtcacttgaatcttcattggatagctcttcctcagatgattcggatgatgattctgattcgaggaacgtgcaagttaaaaaaattaatttcaaaacaagtcatggaccagctggaaattctaatggaaaaccaatgctggatgtggatgacaatgaaataaatacaaagaagtaccaaacacctaaatatgttgaaagtgatgaagacgaccagtaa

SEQ ID NO: 28 - DNA, 2502 bp, Saccharomyces cerevisiae
atgtctaaccaaatatactcagcgagatattcgggggttgatgtttatgaattcattcattctacaggatctatcatgaaaaggaaaaaggatgattgggtcaatgctacacatattttaaaggccgccaattttgccaaggctaaaagaacaaggattctagagaaggaagtacttaaggaaactcatgaaaaagttcagggtggatttggtaaatatcagggtacatgggtcccactgaacatagcgaaacaactggcagaaaaatttagtgtctacgatcagctgaaaccgttgttcgactttacgcaaacagatgggtctgcttctccacctcctgctccaaaacatcaccatgcctcgaaggtggataggaaaaaggctattagaagtgcaagtacttccgcaattatggaaacaaaaagaaacaacaagaaagccgaggaaaatcaatttcaaagcagcaaaatattgggaaatcccacggctgcaccaaggaaaagaggtagaccggtaggatctacgaggggaagtaggcggaagttaggtgtcaatttacaacgttctcaaagtgatatgggatttcctagaccggcgataccgaattcttcaatatcgacaacgcaacttccctctattagatccaccatgggaccacaatcccctacattgggtattctggaagaagaaaggcacgattctcgacagcagcagccgcaacaaaataattctgcacagttcaaagaaattgatcttgaggacggcttatcaagcgatgtggaaccttcacaacaattacaacaagtttttaatcaaaatactggatttgtaccccaacaacaatcttccttgatacagacacagcaaacagaatcaatggccacgtccgtatcttcctctccttcattacctacgtcaccgggcgattttgccgatagtaatccatttgaagagcgatttcccggtggtggaacatctcctattatttccatgatcccgcgttatcctgtaacttcaaggcctcaaacatcggatattaatgataaagttaacaaatacctttcaaaattggttgattattttatttccaatgaaatgaagtcaaataagtccctaccacaagtgttattgcacccacctccacacagcgctccctatatagatgctccaatcgatccagaattacatactgccttccattgggcttgttctatgggtaatttaccaattgctgaggcgttgtacgaagccggaacaagtatcagatcgacaaattctcaaggccaaactccattgatgagaagttccttattccacaattcatacactagaagaactttccctagaattttccagctactgcacgagaccgtatttgatatcgattcgcaatcacaaacagtaattcaccatattgtgaaacgaaaatcaacaacaccttctgcagtttattatcttgatgttgtgctatctaagatcaaggatttttccccacagtatagaattgaattacttttaaacacacaagacaaaaatggcgataccgcacttcatattgcttctaaaaatggagatgttgttttttttaatacactggtcaaaatgggtgcattaactactatttccaataaggaaggattaaccgccaatgaaataatgaatcaacaatatgagcaaatgatgatacaaaatggtacaaatcaacatgtcaattcttcaaacacggacttgaatatccacgttaatacaaacaacattgaaacgaaaaatgatgttaattcaatggtaatcatgtcgcctgtttctccttcggattacataacctatccatctcaaattgccaccaatatatcaagaaatattccaaatgtagtgaattctatgaagcaaatggctagcatatacaacgatcttcatgaacagcatgacaacgaaataaaaagtttgcaaaaaactttaaaaagcatttctaagacgaaaatacaggtaagcctaaaaactttagaggtattgaaagagagcagtaaagatgaaaacggcgaagctcagactaatgatgacttcgaaattttatctcgtctacaagaacaaaatactaagaaattgagaaaaaggctcatacgatacaaacggttgataaaacaaaagctggaatacaggcaaacggttttattgaacaaattaatagaagatgaaactcaggctaccaccaataacacagttgagaaagataataatacgctggaaaggttggaattggctcaagaactaacgatgttgcaattacaaaggaaaaacaaattgagttccttggtgaagaaatttgaagacaatgccaagattcataaatatagacggattatcagggaaggtacggaaatgaatattgaagaagtagatagttcgctggatgtaatactacagacattgatagccaacaataataaaaataagggcgcagaacagatcatcacaatctcaaacgcgaatagtcatgcataa

SEQ ID NO: 29 - DNA, 378 bp, Saccharomyces cerevisiae
atgaacgtcgttagagtctggccattttctacacgaactataccttgtaccactacagtggcagcttggtggacatgttgcgtgacagtcacagcaactggggcagataaagcgacagatgcagatgcggccagtagtgagactttagaaaatttcatctttgtaagttttattaaaggaatggtattgttttatgatgggctgtttcgaggaacgatgataccaagagccaacagatccatcacagcaatatgtaacactctgcgacaagcatatatatatacaaagatccaaagcattgcctttgaacgacgtctatgtttccaaaacattacatatagagaagtgcgtgagatacacgaaatgtactgtaaatag

SEQ ID NO: 30 - DNA, 288 bp, Saccharomyces cerevisiae
atgtccggaaaaccaccagtttatagattaccccctcttcccagactaaaagtgaagaaacctatcattaggcaagaagcgaacaaatgtcttgttttaatgtcaaacttattacaatgctggtcttcatatggtcatatgagccctaaatgtgctggcttagtaacggaattgaaaagttgcactagtgaaagtgccctcggcaaaagaaacaacgttcaaaagagcaatattaactatcacgccgctaggctgtatgataggatcaacggtaaaccccatgattaa

SEQ ID NO: 31 - DNA, 714 bp, Saccharomyces cerevisiae
atggccccaaaaaaagttttactcgctcttacctcatataacgatgtattctacagtgacggcgccaagaccggtgtttttgttgtagaagccttacatcccttcaacacattccgaaaagaaggttttgaagtcgattttgtatctgaaaccggaaaatttggttgggatgagcattccttagccaaagattttctaaatggtcaagacgaaacggattttaaaaataaagactcagatttcaacaagacattggctaaaattaagacaccaaaagaggtgaatgccgatgattaccaaattttttttgcatctgcaggccacggtaccttatttgactatcctaaggctaaagacttgcaggacattgcttccgaaatttatgctaacggtggtgttgtcgcagctgtttgtcacggtcctgctatttttgatgggttaacagacaaaaaaacaggaagaccattgatcgaaggtaaatctatcaccggttttactgatgttggtgaaaccattttgggtgttgatagtattttgaaagccaagaatttggcaaccgttgaagatgttgctaaaaaatatggcgctaagtatttagctccggttgggccctgggatgattactctattactgacggaagactggtaacaggtgtgaatcctgcttctgcgcactccactgccgtaagatccatcgacgctttaaaaaactga

SEQ ID NO: 32 - DNA, 1410 bp, Saccharomyces cerevisiae
atgtttctcaatagaatgatgaagaccaggactggtctttatcgcttatattcaacccttaaagttccacatgtagaaatcaatggcataaaatacaagaccgacccacagactaccaatgttacagattcaataataaagcttaccgacagatcattacatttgaaagaatcacatccagtaggcattcttcgcgatctaattgaaaagaaattaaactcagtcgacaacacatttaagatctttaataatttcaagcccgtggtaaccacaatggaaaacttcgattctttagggtttcctaaggatcatcctggaagatcaaaatctgacacatattatataaatgagacgcacctactgagaacacatacttcagcccacgaattagagtgctttcaaaaaataagaaacgattcagataatattaaaagtggatttttaatatctgcagatgtgtacagaagagatgaaattgacaaaactcactatccggtattccaccaaatggaaggagccacaatttggaaacgaacgaaggctgatgtgggcgtaaaggagccaatgtatatcgagaaaatccgtgaagatatcagacaggtagagaaccttttaaataaagaaaatgtaaagattacggttgacgatgatactatacctttgaaagaaaataatcctaaacaagagtatatgtccgatctggaggttgatttgtgctctcaacatttgaagaggtccattgaactgatagtttctgaagtttttaacaaaaaaatatctagcatgatcaagaacaaagcgaataatacacccaaagagctaaaagtccgttggattaacgcttacttcccctggaccgcgccctcatgggaaatagaggtttggtggcagggcgaatggctcgaactctgcggatgcggattgattcgtcaagatgtgctactaagagccggatataaaccttctgaaacaattgggtgggcttttggcttgggtttggaccgcattgctatgcttctttttgaaattccagatattagactgctttggagtcgtgatgaaagattttcaagacaattctccaagggattaattacttccttcaaaccttattcaaaacacccgggatcatttagggatgttgcgttttggttaccagaagataaaccagatattcatcaagttcatgaaaatgatttgatggaaattatcagaaatatagctggcgatttggtagagagtgtcaagctagtcgatagctttacgcatccgaaaactgggagaaaatctatgtgctacaggatcaactatcaatcaatggacagaaatttgacaaacgccgaagttaacactttgcaagacatggtgtgttctaaattggtaaaagaatacagcgtagaactcagatag

SEQ ID NO: 33 - DNA, 1323 bp, Saccharomyces cerevisiae
atgatcagaacatcatctatattaaaaaactgtaactatagatacatacattgcatccatagatgcttgctgaatgaagccaatttgaaagatagaaaaactcacaacgtagaaagagtttcaaatgaaaagacatttgaacaagctctcgaagaggaacgtaaggtatttggagaactattcgaagctggagcaagggttgaaaacatgaggcacactaatgctagcaaaataattgacaagtattacaatgggctacaggacaactccgagggaactagtgtaaaaaaagaaaaaattgtttttaaccacagtcaaagagctcaaaggaagcttccaaataaggaccatgaattcctgaaggaaacagcaggcaatgactatgtctacgaacgggcagaaccttctgctattagcactaaaacgataagtgagcagacaagaactttgctggaaaagatatttgacgaagataatagcattaataaaagcaacagagaattgctgaatcttaatctgcgcaagggctcgggaatggaagctttgaggcagccagttgcacattccaacgtcaaatttagcgaggaagtgatgcaagaaattggtaataagattcggtaccaaaccacactggatcaggttttagaacctcacattgactacttgagggaagctgttaaaagtgattacgatcttttaagatacctaaagcaatcactagacatttacaagaaaaggaacaaagacctagagttaaagatgaatgcagaatcttcaaacattttcgaagacattagaagtgcgtgtataaataaaccggcagaactaccgaaaccgcttgcaatgactttaccatatattatagtcaaatcgcttagattaggtgactttgatttcccggcagacagaaagtacacattgatctcctatgtttataatgaatgtaaaaacaacatggacgcttcactgtacctaacaatttgtaatgttgatttctacaatttgctggttcagctattgtgggaaaacttccaggagattcgctacttgagacgtgtggttaccgagatgagcgttaatggtgtcatcggaaatattgaaaccgtggatatattggataagattgtcaaagaaatgagaagtttaaatgaggatgtctttcttgaagcaggagaacagctcagtgcagatgaggaagtttcgtccagcgcaaataaaatagtgaatgtaggtgttttatggaataaagacacgaataatgacttactaattgttgaaaattatctcaaaagcctcaaaaaaaatttaactagagacaggtag

SEQ ID NO: 34 - DNA, 954 bp, Saccharomyces cerevisiae
atgagcagcacgaggaagttcaaaaaggttgagaaacctctatcacaaaccagacattattcactctgtatccccacgacattggtatcggactgtcgcaacctctctcaaatcacgcataaggtgtaccaagtggccaagtttgcatcgctgtttaatgtttctgaggttgtcattcttgaggacaattcccaggtcgatgccaccaaaaaaaaaatctccactgctaaattaatactggcacttttacaatacttcgtcacgcctccatatcttcgcaatactgtcttcaacgaaaaattcagaccttatttgaccgccgcttcaaaattaccaagattatccacacttcctttcaccaggtaccaaaagcaagatcacggtcggtacagagaaggcctcactatcaagatgcagaaacccaccttggctcgcaaaaagattggtaaggtattcaagcaaaccaaatatattaatatagggaaaagcaaagcactggctctacaaagtcagctggtgccgataaacgcaagggtcaccatcgacacaataacgaggaaaattgtatctcctcaggaagcttacggtgatttcaccggattggactcgcagtatggctactatactcgtatagcgtcatcgttcacagacttgtttatgaaggggcctttaaaagagggttatacacaatcagtatatgttcccttaaccacgagagacacctctattcccgagttatcatcactgcctaccgccgaaactaatccccacatcttattggtattctcaacgtgggacactcttgctcgagcattcaaactggatcaagatcaattcgtagattgtcaaggtcctcaagagttctttgatgctcagctaccatgccccgtatccaattcggacgttgcagatgcaatccccatgacactgacaactctttcaactgtattttaa

SEQ ID NO: 35 - DNA, 423 bp, Saccharomyces cerevisiae
atggttgtgaaagctattgcacgaaattccataggtcgtaatggagttggagcattcgtgtttccatgcagaaagattactttgcaattctgtaactggggagggtcttcggaaggcatgaggaaatttttgacttcaaagagattagataaatggggccaggagttcccatggatacagtttgaagtgatgaggaagtctggacatcctttgttgagggcagaatacacaaatgggcgtgaaaaagttatttgtgtgagaaacttgaacattgacaatgtagaaaacaagctaaagctattgaaggattctgatggagacattttacgcagaagaacaaagaatgataacgtggaaagcttgaacagtagtgtgaggggtatatggtcgcctctgcatgctgcgaaaaggcatagaatttag

SEQ ID NO: 36 - DNA, 693 bp, Saccharomyces cerevisiae
atgattttaaccacagctagattaaattgtagaccagtcaccgttcctcgtctatttaatcgttcttttagtcaatctttcataattttgaagaaaaaaagttctacccctactgagaaagtcgaagaagatgaaattgacgtgaatgaactgctgaaaaaggcagaaactcaattcaaaaaaactttagaaattcaaaaacagaaaatgaatgagataaaacagggaaattttaatcctaaggtattcaatagtttagtgttcaaaaataacagaaagtttacagatattgctaccacatccttgaaaggtaaaaatgcacttttaataacagttttcgaccccaaagatgtgaaaactgtgatcagtggggtgcttgctgcgaacctgaatttaactcctgaaagggtcccaaataacgatttgcaattgaaagtttcgttaccaccaccaactacagaatcccggttaaaagtagctaaagacttaaagagagtatttgaagaatataagcagtcatcgctaaaagactcattaggaactatcagaggcagtattctaaaggaattcaaaagtttcaaaaaggatgatgccgttcgaaaagctgagagggatttggaaaaactgcataaggattacgtgaacaagcttcatgaccaattccagaaagttgaaaaaagcattgtaaaatga

SEQ ID NO: 37 - DNA, 408 bp, Saccharomyces cerevisiae
atgatgtttaatatttaccttttcgtcacttttttttccaccattcttgcaggttccctgtcagatttggaaatcggtattatcaagagaataccggtagaagattgcttaattaaggcaatgccaggtgataaagttaaggttcattatacaggatctttattagaatcgggaactgtatttgactcaagttattcaagaggctctcctatcgcttttgaacttggcgttggcagagtaattaaaggttgggatcaaggtgttgccggcatgtgcgttggcgaaaaaagaaagctgcaaattccaagttctttggcctacggagaaagaggtgtcccaggcgtcattcctccaagtgctgatttggtgtttgatgtcgaattggtagacgtgaaatcagccgcctag

SEQ ID NO: 38 - DNA, 324 bp, Saccharomyces cerevisiae
atgaaccacttgacaacggtcttggccgcacgtaaaacctgtgtggtgcgagaagcaacgaatctgtatacagttgatacagaacgtcggatcaacaaaatggggcgtgatgaagaaaaaaagtggcgatcccgtggaggacattggtgcatggatgattcaatcatacaatttacgacatttaaccaaagttgcactctgatgggctcaacgactgctactcgtgccaataatgaaactgaaaagcatgaagccatcactgtttttttcgtggtctttctatatataaacaaagcctctgtaaagtttttttttttcgcataa

SEQ ID NO: 39 - DNA, 2121 bp, Saccharomyces cerevisiae
atggacaagctaactccatctcaatggaaggtaataaataagtcatatgaaccagcgtctacgataaaagtcattgcgggcccaggctcaggaaaaacgctaacgctactatataaagtactacatttaattactgtggagaatattaagcctgaagaaatacttatattttcgttaacaaacaaagcagttgatagtataatagaaaatttactgtccattttcgaaaactcgcatacaaataaggaaattgtccatcagattgggtgttacacagttcatgggttggctaatagaatcgttgtagagaatgaggggatgataaacattatagaagagattggatggagaggattaatgaagttgttaccgccgtcaaagagaaccccccatcatttcagatcatataaggaactcgagaaagttgttaaggattacaagctaaacaacgcaaaaaataataaccccgtcattgagaaactagttgaactgatggataactgtaaagtgatgaccaatgatgatttgatcattcgcgcaaaaaaatacttagagcttgattcttctgattcagatgcatcgtcttttactcaagatctccgcaataaatacaaggttgttcttatcgatgaatttcaggacctctatccaagtttagcacccttaattaccatgatttgcaaaggcaagcagctcattatgtttggtgatacaaatcaaagcatatacggatttttggggagtaacaacgagattatgtcacaattagacaatcttcacccaaaaaattccacgactgtcctgaaattatttgataattttaggtctacccccgaaataatctctctagcatcaaaaattatcaatcgtccactagcagagaaacagattatcgatgatactgatgaaacaccatcagagctagttcgcaagctaccgagtggtgtttcacctcaaataatgacatttgatgatctagccgcagaatctgaatttattattgataaaattacccaattgatttgttcttctgcaaagttttccgatatagcaattctatcaagaacaaattcacatctaacggctatagccagtattttgaagaaatatggtattccttatcaaaaactaaagagtcaacctgattggatggacgatttacgaattcaattccttttagatattctaaaagtgtgttctttagcctccgacgaaaagcataatcgtgagttcaatacgggtgataaatggcaaagtaacttcagcattttggtcacaatgagcgctctcaaagggattggcgatgcttcaatacaagccttgtataaagcatgcagcctgaaaaatctgtcaatttggaaatacctaactatggttcccaattttgaatggccattgggcctctccataaagaaaaaaatggaaaattacacttcaaatttatatgaaatgatcgaaaatgatcaagttcatcaattagatgatccgatggaacttttagaaaaagttgctagcatcacaaataacttaaacctaaatcctacctacttccaatccttaagtgatgctcaatcatctttagagtttaaaacacatctacaagagatggcacaagtaatgaaggtctcaaaatctaataagcccccgggtatttcatttgttaaatggtttctagagacatattttgatcaaacgatggtctttcaccagtctcagcaggcattacaaactaccggtccggggacagtaaaactatctaccatccattctgcaaagggtttagaatttcccatagtatttctaacaaacgggagcatgtccaatttcccaatggatacaaacgccctttatgtggggataactagggcaagaaacttgttgtacatgtgcaacatgaagcacgagagactggtcagtaaatcatcaccatactcaagaaacataatgtctaataatttattctggacttactacaataaggatttgaaacgatctgtatgtgacgttaaggtgacacatggttataatgtgcagcgatacaatcagttacgtaaaaattttggattttatagagcatattcttcactaagaggttgtaagagcgttttcagacgtatatag

SEQ ID NO: 40 - DNA, 1686 bp, Saccharomyces cerevisiae
atgtcgctttttttcaagccagtaatttcaccacagtggtctttccccgtgcttctcaagataggagtgcgttcttatgcgggaggcccaagaacaaagcataaaggaaactctccattagcttcagtgcctacaggcagtagtaataaaaaccgaaaacagaaggccaaaggcaaaaaaggtaacaaaaagaatgaccctgaccaagcattcaactttggagagtatggtggattgaaaaaagacgtcgagatgaatatggacagtacgaataaactcattcaaaaaatatcgaatttcgaccaattgcttatacttccacctgtcagagacgctgtgaaagaaatcatttccaaagagtccctgaagctacaagatagtaggaaaaaaaccagtgaaaatataataccgagtcccattcaaacagtggccattaagagaatatccaaaaatttaatggatccaaagttacaaatacatgcgattgccgctgagacaggttcaggtaagaccatggcctacttgatacctttaatagactatttgaaaagacaggagttggaaactcccgaattatgggaaacattaagaaaaaacgttctgattcggtccatcatactggtgcctacgcatgaattggttgaccaagtatacgaaactgtttccaagacgaaaaccttgttaggcttaaactctttcaagtgggacaaggccacatcatatcgtgatttattagagaacattaaaaatagaatagatattttagtcactactccgggaaaactactgaacctgttctccataagaatgataactagacctgataaagtcttgtccaaagtgggtttcgtagtactagatgaagctgacacacttctagataggtcatggcttgaagaaacgcactccgcaataaagagaattcctaacattaatcaccttattttctgttccgctactattcctcaagagtttaataaaacaatgcaaagactatttcctacagtggttcctataatgacacccagactgcataagttaccattcgctttggactttaaagttataaattctgccttaagtcctttcaagggatccaagatcaaagcattggcacaaactttatatgctatttcaaatgatgacacggaaccagggttcgagaaaagatgcattatctttgttaacgaaaaaaagaatgttcctgaaattgttaacctattgaataagaaattcggtcataatgccatcggtttaactggtgaagatacatttgaagaaagatccgagaaaattatgccctttttatcaccaccgcgaccactttcggaggtggttgctcagagtacttcacctcccacttccttgaagaaatttgaaattcctgattccaacatcgtcatcggcaaattgaaaaacacaaattctaatggtaccgcacccagtaacaagtctttgcatgtactcgtaaccaccgacctcatggcaagagggctcaatttcaaaggtgtacggaatgttgttctctacgatgtacccaaaacgtcaatagacctgatacaccgagtgggcagaactgctagaatgaaacagggcggccgcgtatttatgctaactgacagtaaaacaaaatcttgggctaaagctcttccaaagatcatcaaaaagcaccagagattatcatga

SEQ ID NO: 41 - DNA, 6837 bp, Saccharomyces cerevisiae
atgtcatcggaagaaccccatgcatctatatcctttccagatggctcacatgtccggtcgtcctccactggtacttcatctgtgaacacaatcgatgctaccttatccagaccgaactatattaagaaaccgtccctgcacattatgtcgacatctacgacgtcgactacgacggatctagtgacaaacccaattctgagcaatatctccgtgccaaaaataagcccccccacatcatcctcgatagcaacagcgacatcaacaagtcacgtgaccggcactgcgtcgcattcgaacattaaagctaatgctaacactagtaccagcgtgaataagaaaaatttgccgccaacaactagtgggaggataccctctagcaccatcaagagatatccgagtcgctataagccttctcactcgctgcagctgcccattaagaacgactccaactttaaacgatctagcatatatgcctccaaatccactgttactgctatccccattaggaacaatcggccaatttccatgcagaattcctatgcgcgcactcctgacagcgatcatgacgacgttggcgatgaagtgtcttcgataaagagtgcctcttcttcgttgaccgcatccctgtccaagagtttcctgttcgcattttataacaaccgcaagaaggacaagaccagtaacaatggtgtcctctcaaaggaatattggatgaaggacgagagttctaaagaatgtttcagttgtggtaaaacgtttaacactttcagaaggaaacaccactgcaggatatgcggccaaattttttgcagcagttgcactctcttgatagatggtgatcgattcggctgtcatgccaagatgcgagtatgttacaattgttatgagcatgcagacacatacgaggactccagtgatgaagagaatgattctacaatgcaattgaatgagccaaggtctcgctctcgctctcgttcttccaataccaatccatattctcattcacattcgcatttgcatctcatttcacaggacaaccataacggtaccgatctccatgatccagtagcagcaacggataaccctcaacagcagaatgaggtctacctattaaatgacgatgacgtacaaagtataatgacctcaggtgaagattctaaattgttcatttctacacccccaccaccaccgaagatggccatcccagcaacaaagcaaggcggatcgctggaaatctcattcgattcggaaaacgatagggctttgcattatcaagatgacaaccccggtcgccatcatcacctcgattccgtgcccacaaggtacactatcagagatatggacaatatatcacattacgataccaactctaattccactttgaggccacattataacaccaataacagcactattaccatcaacaacctcaacaacactacttccaacaacagcaactataacaataccaatagcaacagcaacatcaataatcctgcgcattccttaagaaggtctattttccattatgtaagcagcaatagtgttaacaaggacagtaacaatagctctgcaacacccgcttcgtctgcacagtcttcttccatactggatcctgccaatcgtattatcgggaattatgctcatagaaattacaagtttaaattcaactacaattccaaaggtccttcgcaacaaaatgacacggcaaatggaaataatgataataataataataataataataataataataataataataataacagtgccagcggtattgctgacaacaataacattccctcaaacgacaatggcacgacgttcactctagataagaagaaaagaaacccactgacaaaatctaaatctacgtccgcttacctagaatatcctctaaacgaggaagactcttctgaggacgaaggttccatgtccatatattctgtattgaatgatgaccataaaactgacaatccaattagatcaatgaggaactccacaaaatcttttcaacgagcgcaagcttcactacagcggatgaggtttagaaggaaaagtaaaagcaagcattttcccaacaattcgaaaagttccatttatagagacttaaatttcttgaccaatagtactcccaatttactttctgtagtaagcgacgataatctatacgacgattcttctcctttgcaagacaaggcctcttcatccgctgcaagcagactcactgacagaaaattcagcaatagcagtggcagcaataataatagcaatagtaatagcaatatcaacaccgatccttggaagagaatcgctagtatatcgggattcaaactgaaaaaggaaaaaaagagagaattaaatgaagtctctcttttacatatgcatgccctattaaaacaattattgaatgatcaagaaatttccaatcttcaagaatggataacccttctagatggcgctttgcgtaaagttttaaggaccatcctgaacgcgagggatttgaacacattagattttagacaaacttacgttaagataaaaagaattagtggtggatctcctcaaaattcagaatatatcgacggggttgtcttttcgaaagcattgccgtcgaaaacaatgccacggcatttgaaaaacccaaggatactattaatcatgtttccactggagtaccaaaagaacaataaccactttctaagtatagagtccgtctttaggcaagagcgcgaatatttggataaacttgtttcgagattaaagtcattacatcctgatattatatacgtcggcgccaatgttagtgggtacgcgttagaactattaaatgactcaggcattgtcgtacagtttaatatgaaaccacaagtcattgaaagaattgccaagctaacagaagcagatattgccatttctgtggataagttggctacgaatattaagatgggtgaatgtgaaacttttgaagttaaaagctacatttatggaaacatcagtaaaacatatacatttcttcggggctgtaacccagaacttggtggaacaattttgttaagaggtgatagtcttgaaaacttgagaaaaatcaaacaagtctctgaatttatggtctacgcaattttttcattgaagttggaaagttcattttttaacgataatttcatacaactttctacagatgtttatttgaagagagctgaatccaaaaaactgcaagttttcgaaggttattttgcagatttcttgatcaaattcaataacaggattttgactgtatcaccaacggtagatttccccattccctttttattggaaaaagccagaggcttggaaaagaagctcatagaaaggatcaatcaatatgaaagcgaatctgatttagatcggcaaacgcaactaaatatgcttcaaggcttagaatcgactatcactaagaaacacttaggaaatttaataaaattcctgcatgaaatggaaattgaaaatttagagcttgagtttcagaagagaagtcgtcaatgggaggtttcatattcatcatcacagaacttacttggtacgggctcacaccagtctataacagttctctactcaatggtttcaacaaagactgccacaccttgcgttggccctcaaattgttactattgactatttttgggacagtgatatttccattggtcagtttatcgagaacgtcgtgggaactgccaggtatccctgccaacaaggctgtaatggtctgtatttggatcattatagaagctatgttcatgggtcaggaaaagtagacgttttaatcgaaaaattccaaactagattaccaaaattaaaagatattatcctcacgtggagttattgtaaaaaatgtgggacatctactccgattttacaaattagcgaaaaaacctggaatcattctttcggtaaatatttagaagtcatgttttggagttacaaagatagcgtgacaggaattgggaaatgtcctcatgatttcactaaagatcatgtcaaatactttggttacaatgatttagtggttcgactagaatattctgatttagaagttcatgaattgataaccccaccacgcaaaataaaatggaaacctcatatcgacatcaaactgaaggtggaactatactataagattctggaaaagatcaataatttctatgggagtgtcctaagtcggttggaacgtattaaattggatagtatgactaaagataaagttcttagtggacaagctaaaattatcgaattgaagagcaatgctacagaggagcaaaaattaatgttgcaagacctggatactttttacgcagatagtccttgcgatcaacatttaccattgaacctggttatcaaatctctatacgataaggctgtaaattggaactctacatttgcgatatttgcaaaaagctatttgccttctgaaactgatattagtcgtatcacggccaagcagctgaaaaaactgttttacgattcttcaagaaaagacagcgaagacaaaaaatcacttcatgatgaaaaggcgaagacgaggaaaccagaaaagaacgaattaccgttagagggattgaaagatgtcgaaaagccgaaaattgatagcaagaatacgacagagaatcgtgacagaacgaacgaaccacagaatgccgtgacgataaccacttttaaagacgacactccaattattccaacaagtggtactagtcacttgacagttacaccttctgcatcgtcggtttcttcttctttgacccctcaaaccgaagaacgtccgccaataagcagaagtggaacaggtatatctatgactcatgataaaagcactagacctaacataagaaaaatgagtagtgatagtagtttatgtggactagcatcacttgctaatgaatatagtaaaaataataaggtgagtaagctagctacatttttcgatcaaatgcattttgatgccctgtcgaaagaatttgaactggaacgtgaaagggaaagattacaacttaataaggataaataccaagccatacgattacaaacatccacaccaattgtggaaatttataaaaatgtcaaagacgcagttgatgaaccgcttcactctagaagttcaggcaataatttgtcatctgcgaatgttaagactttggaggcacctgtgggagaacattcccgggccaataattgtaatcctcctaatttagatcaaaacttggaaactgaattagaaaattccattagccaatggggagaaaacattttgaacccctctggaaagaccaccgcaagtactcatttaaattctaagccagtagtaaaggaaacttcagaaaatccaaagtcaatagttagagaatcagataattcaaagtcggagccgcttccacctgtaataacaacaacaactgtcaataaggtagaatccactccacaacctgaaaaatcattattgatgaaaaccttgagcaacttttgggcagatcgatctgcgtatttgtggaaaccccttgtttatccaacctgtccttcggaacacatttttactgatagtgacgttataatccgcgaagatgaaccaagttccttgatcgcattttgcttaagtacttctgactatagaaataaaatgatgaacttgaatgtccagcaacaacagcagcagcaaacagcagaagctgctcctgccaaaacaggaggaaacagtggaggaactacgcaaaccggggatccttctgttaacatttctccctcagtttctacaacttctcataataaggggagggactcagaaatttcatccttagtaaccaccaaagagggcttgttgaacacccctcccattgaaggcgcccgagataggaccccacaagaatcacaaacgcattcccaagcaaatttagatactcttcaagaattagagaaaattatgacaaaaaaaacagccacacacttgagatatcaatttgaggagggtttaacggtaatgtcatgtaaaattttcttcacagaacattttgacgtgtttagaaagatttgtgattgtcaagaaaacttcattcaaagtttgtcaagatgtgttaaatgggattccaatggggggaaaagtgggagcgggtttttaaagactttggatgatagatttattataaaggagctgtcacacgcagaactggaagccttcataaaatttgcaccaagttattttgaatacatggcacaagcaatgttccatgatttgcccacgactttggctaaagtatttgggttttatcaaattcaagtaaaaagctctatttcaagttccaaaagttataaaatggacgttatcattatggagaacttattttatgagaagaaaacaacaagaatatttgatttgaagggttccatgagaaatagacatgtcgaacaaacaggtaaagctaatgaagtgttgttggatgaaaatatggtagagtatatttacgaatcacctattcatgttcgcgaatatgacaagaaacttttaagagcttctgtttggaacgacacattatttttggccaaaatgaatgtgatggattattccttagtcattggtattgacaatgaagggtatactttgacagtaggaatcattgatttcattcgaacgtttacatgggacaaaaaactagaaagttgggtcaaagagaaaggcctagtagggggggccagtgtgatcaagcagcctaccgttgttacaccaagacaatataaaaaacgttttagggaagcaatggagaggtatattttgatggttcctgatccgtggtattgggaaggaaattaa

SEQ ID NO: 42 - DNA, 384 bp, Saccharomyces cerevisiae
atgccacagtcttttacgtctattgcgagaattggtgactatattttgaagtcacccgtcctctccaagttatgtgttccagttgccaatcagttcattaacctcgcaggttacaagaagttagggctcaaatttgacgacttaattgcagaggaaaatcccatcatgcagaccgctttaagaagactccctgaagatgaatcttatgccagagcatatagaataatcagggctcatcaaaccgagttgactcatcatttactgccaagaaacgaatggatcaaagcccaagaggatgttccttacctgttgccatacatattagaagctgaagctgcagctaaggagaaggacgagttagacaacatagaggtctccaaatga

SEQ ID NO: 43 - DNA, 1098 bp, Saccharomyces cerevisiae
atggctcaaaattttggaaagattccttctcacaagtcttacgtattgagcttatatcggaccgttctcagaaacattccaaagtgttgccactcatatgcgtttcaatatgaaataaaaaaaacactttcaatacaattgttcaaacacaagcatgacaagtcaagttggagtgtttatactttactcaatgaattcagtttgttgaacaattgccttttggaaggtaaattacaagaaatcaaaaacttaatgaagccattaaaaaagatgaaaaaacaactgaaaacaacgaaaatcctcaactctttgacttcattaggggatgtaaagaccaacgacccagaagaagtaagaagatttcatgttctaagtgcttacattaagcgaaaacaagatttgggccttttacctgcatacataccaaaaacgtatcagcacaaactcctattaccattagcattgaacgaacatgcgtgcctgaaactatttcatattcagcaaaaattgaaaaatggcccaccttctgctgggctgtcatatacaaaagaaggaaggaatcaaatatggtttgttcggtcaccaattaacaagggcagacaacaatcgaaaaagctgggaatattgatacgaaaagaaaggaaggattctcagaaaaatatcgataatctcaatttttgcgaaattaatgctgcttgggcattacatgaggccatttgggaggaatatctagagtcgaaaaagattattaaagtaaacttaccgaaatacttggaatacgctgcaaatatcccaaagtctactaaatgtaatccttcgagtcaatatcagaaagttaaagaatgggtagatcccgtgagagaaataatgttcgaattacattccaaaagttttcaaagagtggagtactttaacaaatataaggaaaagcttttaaagaacggtggccaactagcatattttgataaaaagtcaaaagaaatgtacgccaaacggttgacactattcagaaaaatgagcaaagaaacattaccatatgtgacactctttatagaagggagggacttgccaagtgttttggcgaaatacggtttttaa

SEQ ID NO: 44 - DNA, 1026 bp, Saccharomyces cerevisiae
atggcagtaaaacataaatccgagagccttaagcacgaagagggggctgccaaaaaggcaaagacaggcctattgaagctaaaaaaaatcatggatatagaatctaatgtggtcaaatattccatatgcataccgacaactgttatcgacaattgtaataatttggagcaagttacatttactgcatatcaaatagctcggacagctgtattgttcaatgttcaagaaatcatagttctagaccagtcaaaagataaaaagcatgaaaaaaaatcaagatcaaaggagactatctctgattgtttattacttgccacactattacaatattttgtaacgcctccaaacttactggatactacattcaaaaaaaaaaacaagttatatttaaagtgtgcatctacctttccgccattgaaccagctaccttttatgaacgcatctgccgaacaacactacaaggaaggcttatcaatcgcacgagatagctcaaaaggtaaatctgatgatgctctaacaaatttggtgtatataggaaagaatcaaattattacgttatctaaccaaaatattcccaatactgcgcgtgtaacagttgatacagagcgcaaagaagtagtgtcacccatagatgcttacaaaggaaaaccactcggctatcatgttcgaatggcaagcactttgaatgaggtgtccgaaggttacacaaagatcgtttgggtcaacagtggtgacttccactacgacgaagagttgtcaaagtaccacaaggtagaaacaaagctaccatacatcgcaaaactgaaaaaaagttcaacatcagaaaaaccctgtaatatcctgcttatcttcgggaaatggggccatttaaaacgttgtttcaggagatctgacttagaatcctcttctttgcatcattacttttctggtcaactgcaattccctgcttccgtaccccagggaaatatacccattcaagatagcctacccatagctcttaccatgtttcagcgttgggcaagttga

SEQ ID NO: 45 - DNA, 360 bp, Saccharomyces cerevisiae
atgccgcatttagccgccgaagcgcatacttggcctccgcatatttcacattcaacactttcgattccgcatccaaccccggaacaccggcacgtatttcataaaaaggacgtgaagaacaaaaggaacgaagaaaaaggcaataatttactctatgtgttatttagaactacggtgataaagagctcgttccgatcactaagtacggccggaagagagctgttgtttgttgtccatcaagggcacatcggcaccggcctcatcgtcttcatcatatgctggaggctgtgcttgagattcctctgcagggtgagcttccaggtcacggtctacggcgggcgcagtcgcatgtctgcgtga

SEQ ID NO: 46 - DNA, 1506 bp, Saccharomyces cerevisiae
atggttacaaatatggtgaaattgaggaatttaaggagattgtattgttcatcccgtctcttaagaactatacagaacgggagaatttcttctgtttcttcaatatcgttgagtaagaaatatactacaaaatctgccaaagaaggtgaagaaaacgtagaaaggaagcatgaggaggaaaaaaaggatacattaaaaagttccagtgtaccaacttcacgaatatcgagattgttccattatggctcactggcagcaggggtgggcatgaatgccgcagcaaaaggcatatcggaagttgcaaagggcaattctcccacttggaaatctttgattctatcagattcgaatattgacagaattaccaataaattttctaaaatgagaggtgttgcccttaaaatagggcaactattgtcttttcaggatgagaaagtgttgccaaaggaattatacgaaattttatcaagggtacagaatagtgcaaatcatatgcctcaaaggcaattggaaaaggttatggccaaggaattaggtgctaattggaagacaaagttttccaaatttgacaagatacctatggcagctgctagtatcggacaggtacatgcggcagagttgcccagtggtcaaagagtggttgtcaaaattcaatatcctggggttaaagaatccattgattcggatttgaatagtttactaatgttacttacagcctcaagtttattaccgaagggattatttttggataagacaattgccaacgctagaactgagttaaaatgggaatgtgattacaacagagaggctagagctctacaaaagtttgaggcgttgctaaaggatgacccagcgtttgaggttcctcatgtgtttcctgaatatacaaccgataatattataactatgacacgtatggagggaactgaaataatgaaattgcccaaggcatcacaagaaaccaaaaattttatttcggagaatatcatgagactatgtctggaagaaattgccactttcaagtatatgcaaacggatcctaattgggcaaatttcctctataacggtaggacaaagaaaattgaattattggatttcggtgcctccagaccttttgcagaagattttattctaaaatatagaaaacttttaacttacgccacattgagagataggaaaggggcgtacgaaatgtctgtacaactgggctatctcacaggcttagaatcgcagtccatgaaagacgcgcacgtcgatagtgtacttacacttggtgaaccttttaggggtgatgtagataaatctttcgatttcaaggatcaaacagtctcagacaggattagaggtaatatcgggttaatgttgaatgaaagactatgtccgccaccagaggaaacatactcgttacatagaaaatttagtggcatttttcttttgtgtgcaagaatgggtgcgtctgttcattgtgcaaagctttttaaagagatttttgcctataaagtttaa

SEQ ID NO: 47 - DNA, 447 bp, Saccharomyces cerevisiae
atgttatcaagggccatattcagaaatccagttataaatagaactttattgagagccagacctggtgcttatcatgcaactagattgactaaaaatacgtttattcaaagtaggaagtattctgacgcacatgatgaagaaacctttgaggaattcaccgcaagatacgaaaaggagtttgatgaagcctatgatttgtttgaagtgcaaagagtgctcaacaactgtttttcctatgatttagttcctgctcctgctgttattgaaaaagctttgagggctgccagaagagtcaatgacttacctaccgcaattagagtatttgaggctttgaaatacaaggtggagaatgaagaccaatacaaggcctacttggatgaattgaaggatgtcagacaagaattgggcgttcccttaaaggaagagctatttccaagctcttcttaa

SEQ ID NO: 48 - DNA, 1557 bp, Saccharomyces cerevisiae
atgattactacagtgcaagaaatcagcaaatggagaaacctgtgcttcattaggatgcaatctcgcaaatggtacccagtacttaaaaagactcctttagtagcggacggtcgaaagatcattaaacatgccgataaagttcctcatccggaagaaataattcacccattttaccaaccaacagcaatagaacaattcacggcatgcgctacggaatacaatcctagcctgcttgatggcaaaaaaattgctccatcactgataaaacacccggtgagcctaaaaaccatactagttgatagcaaattaaagtttgatgatattaggggagtgaataaatggttgatggaattcgttgcaagacgacaacatcaaagaaatattgtacttacacctgcgagcaaaagcgttagatcttttcacgttctgcatttgtcttcaaccgatattgccaaattaagaggtcttgaaaatatattatcagagattgaaaacacgaatgatttgcaatccagggtcgagtccgtaaataatgaactacagaatatttttgaccgcgacagtaaacagacaagattattttgcgagaatatactggcttatttgataaagaattatggaaactcgactgaaaaacttatactactaataaatgttactgagatgcaattatattcacgtttagaccaaatgaaggccatgaacattattttatataatatcctttgtaaagtggaagcaaatgaaaacccaccgtacagtcccacattggttaccgcactagaaaacctattggcagcgatcaacaacagattttttccagggcgttgcgaaaattcattgcacccaatagtaattgaacaactattatcatatttcattaaaaccggcaatttgaatgaaagcaaaaattttttgggccatttaataaaaaagggtattttaccagaggctactatcattaatagatatttggaggcaattgatgttcatttcgataaaagcactaagatttttgatataagatccaaatttgcattcatagcagatcttgcacccataatagaaaattatggaacaatagacctgttcaaatttttgatacctatgtgccggcatttcgacgaattgtgttctctgctaaatattattcgaaaatccaataacgcaaaacgggctgtggatagcaccttacctatttttatcaaaaaagtattaacatttactaaagatccaatgatcaactccggaaatctatccactgttttcaatatagtgagcccaatatatggacaaaacgtgcccagcgaattcgtggagaaatttatactgtcttttgcacttcaaggaaattacactatgatggctcatatgatagatacatataagatcaaactaagtcataaataccaactacaaattattagggctctcaaaaatagtgagagaaatcacgcgcttaagaatacgggcgctgtaggttataataaggaatttaaaaagtacttcatcgagaaatatttgaattgcactgaacgagaagctttacgcccataa

SEQ ID NO: 49 - DNA, 318 bp, Saccharomyces cerevisiae
atgttggcgcgcagtttgggttacaggttgatatcgaccagtcgtatcttatataataaacccacggttaagtcagtggtgtcgtcgtgtcctgcgggaacatcgcttaacttaaacatatggaagagcggtaaagacgcagtggcactcgaggacaaggagtatccaaattggttatggagcgtattggatagtgatcacgttgtcgaacatgcggctgaggacccagagggacaggctctcttaaagagaagaaagaacataaggaaggcgaacaggcagcgaatcaagcaaaacaatttcttgagccaactttaa

SEQ ID NO: 50 - DNA, 1932 bp, Saccharomyces cerevisiae
atgttcgggatagtttatctgaagaatcgcagtttactctgcaagaaccccttttcttcttaccctcgttatggcttcatgccctcgttcgacacccagttcagcaaccaatttcgtaaattagaaattaatattggacggaaaagatacagctcaaaaaccttgaatacaaaatatactgaccaacccgaaggcccaatttatcctttggatgtactacgcctagacatctcaaaagcactgcatgatatatccggaatagaccactcgctaatactaaatgcgcttgagtcgacaaatagtatggatagaggtgatctactgctacctcttccaaagataaaagtggctgaccctgtggccgttgccaaccgatgggccattgaactaagcacgcatgggtgtattggaaaggtttgtgcaaaagggccatttcttcagttcttcctggaccagcggtatttgatacaatcaacggtaccaaatatattacttcaaaaggggaaatatggtcagaaaaaatctcgtcatcagaaaaaagttgtagtagaattttcatcaccaaatattgccaaaccattccatgcaggccatttaagatcgactataataggtggattcctttcaaacttatatgaagcaatggggtggtccgttacacgtatgaactacctaggagactggggcagacagtttggtttgttagcagtcggttttaagagatacggcgatgaaaagacgttacaaaagcagccaattcagcatttattcgacgtctacgttaaaatcaacatggaccttgccaaggaagagataaacggaaactctaagtgtgggattagcggtgaggcccgtagttttttcaaaaacttagaaaatggggatgagaatgccattaagatatggaataggtttcgctcgttatccatccaccactatatccaaacttattcacgtttgaacattaattttgatatattctccggtgaatcacaagtgtctaaagaatctatgaatgaagccttagatatatttcgcaaaaacaatcttgttaaagaaatagacggtgcccttgtcatcgatttaacccaatggtcgaaaagattgggcagagttgttgtacaaaaatcagacggaacaactttgtacttgacaagagatgtaggggcagccatagagcgtaaaaagaatctacattttgataaaatggtgtacgtgatttcctcacagcaagatttatacatgtcccagttttttatgatattaaaaaaaatgaattttgaatgggcaaaagacttgcagcatataaattttggtatggtgcagggcatgtcgaccagaaaaggaaatgtggtattcttagataccatactagacgaggcaagggataaagctctgcaaattatggaaaataacaaaatgaaaatatctcaggtagataatccacagcgtgttgcagatttaattggcgtatctgctatcattattcaagatatgaaatcaaagcgtatcaataactatgaattcaactggaacagaatgttgtctttcgaaggtgacacagggccctatttacaatacacacattccagacttagatctttagaaaggacctccagcgactttacgacggacatgctaatacacgcagatttttcaaatttaaatgaaccacaactagttgagcttgtgaggttgctagcgcagtatccagatgtccttagacgagcattcgaaactcaagagccggccacgattgtaacatatttattcaaagtttgccatcaggtctcctcgtgctataaaaaaatatgggtttccggaaaacctgcagatattgccataccgcgactcgccgtatattcagcaagtagacaagtgttacacaatgcaatgtcgcttttgggactagtcccggttgatagaatgtaa

SEQ ID NO: 51 - DNA, 681 bp, Saccharomyces cerevisiae
atgaaggtaaaccattcaatttcaagattccgccccgcttcatggttcgaaaagacaaagataatcccaccccaggtatacatctttaggaacctagagtatggacaagtactatattctcaatttcccaacttttcgcaaacacaggtggataagctgtttgtgagaccaaactggagcaacagaaagccatcattgagaagggacatctggaaatgtatgtgtgtagtgaacttgcaaaactataagcagagtgtccatctataccagaacctctgccggttgagatacctccgtgatgtggcacagcgtaaggagagtgacaagctaagaaaaaaggactctaacgggcacgtctggtatagcggacagtatagacctacatattgtcaagaggcagtggcagacttgcgggagtccttgttgaaggtgtttgagaatgccacaccagcagaaaagcagacagtacccgccaaaaaaccgtccatatactgggaggacccatggaggatgggtgacaaggacaaacattggaattacgatgtgttcaatgctctggggctggaacacaagcttattcagcgtgtggggaacattgcgagggaagaaagcgttattctgaaggaactagctaagctcgaatcacatcctacagagcagacggaagtgtcttcccagtag

SEQ ID NO: 52 - DNA, 795 bp, Saccharomyces cerevisiae
atgtcgtcactgcttaaattacattgtattagaccattgccgcaaaggtcggtctggctctctggctacaagcagaaagccagatgtattcacagttccgctgcaaacggggactttatgtcatggttcaagaggaagaagcaagaagagcaccaagagcctgtcaaggacaccaaacagttgataaaggatattgaagaaggcactaacgaggcttcctctcaatcctcttccaataacaaaaacagattggagctaataccagaaaattttattggcgaagggtctagacggtgtaagagacagaaggagttgaagcttgcggtctcctctgcgccattcaatcagtggttatccagggataagattacgtctgataatcagttagatgatatgatattgcaggcaacggaaaaaacattgggcaaagtagaccaggacgtccaattctctgatttggtagctaaatttcagttcaccaaatttttgcaatctaaatctggctatctaattcctgattatgaactgaccactttaagcacacccttgcagtttaaaaggtatattaaggaaaaaattttgccatctgccaacgatcctaaactcgcttataaagaagctgagccaaatgctatccacccattttctgataattacgcgtcaccaaacatatatgttgttaacgatgttacttctaaagagcaaaaaagtaaatatgatactattatgaaagaaattcaaaagttagaggatgatgcaacaagaaaagctttggaaacagcaagatccgcttag

SEQ ID NO: 53 - DNA, 534 bp, Saccharomyces cerevisiae
atgttgaagtctatatttgcgaagcgatttgcttccacagggtcatatcctggttctacaagaattactttgccaagaaggcctgcaaaaaaaattcaactggggaagtcacgaccggctatttatcaccaattcaatgtcaaaatggagttgagtgatggaagtgtagttatccggagatcccaatatccaaagggtgaaattagattaattcaagaccagagaaataatccgctgtggaatcctagcagggatgacctagtcgttgtggatgctaattctggtggtagcttggacagatttaacaagaggtatagctctttgttttctgtcgattcaacgaccccaaattcaagctctgaaacagttgaactatctgaagaaaataaaaagaaaacacagattaaaaaggaggagaaggaagacgtttctgaaaaagcgttcggtatggatgactatctatctctgttagacgatagtgaacaacagatcaaatcaggtaagttggccagtaagaagagagacaagaagtga

SEQ ID NO: 54 - DNA, 2568 bp, Saccharomyces cerevisiae
atgtctgaaggaaaacaacaattcaaagacagcaataaaccgcacaaggactccacagatcaggacgatgatgccgccacaatagtaccacaaactcttacttactcacgcaacgaaggtcattttttaggcagtaatttccatggagtaactgatgaccgtaccactttgtttgatggtgaagaggggcgtagggaggatgatttgttgccctcattgcgttcctccaactcaaaagcacacttaatttcttcacaacttagccagtggaattataacaacaatagagtacttttaaaaagatctatattaaagacacaagcttttatggatcaacttcaagaagaaaataatattcgcccgatttttattgctgcaaatgatgaacgggaaaaattacatgttctacaactgaatatcaagctagatggtcagtacaataccaaagaaaagaacggtttcaacattgagaaaaaagctttatcaaaattatttcactctcagatcgtatcagtgacaaatcatttaaacgctttgaaaaaaagagtggatgacgtttcatctaaagtattcatcacaggtgatgtaaatactggtaaatcagctctttgcaactctctattaaagcagcgtttgctacctgaggatcagctaccatgtaccaatgtattttccgaaatactagaggctcgggaaaatgacggcatagaggaggttcatgccataccactaaacattgctccgactctcaaggaagccattgatatgtattcaatacaaaatcccaagacttatgaaatacacactttgaaagaacttcccgatttggttccccaaaacggaaaatatgctttactgaagatttacataaaggacgataaaaggcctgcctctactagtctattaaggaatggtactgttgatatttcattaattgactcccctgggttgaacatggactcgttacaaaccgccgaagtaatgtcccgacaagaagaaatcgacttagttatatttgttgtcaatgcagagaaccagctaacactatctgccaaggagtttatttctttagcatctcgtgagaaaaaattaatgttttttgttgtgaaaaaatttgacaaaatcagggacaaacaacgttgtaaggagttgatcctaaagcaaatccgtgacctgtccccagaaacatataaacgtgcagctgattttgtccactttgtttccaaaaacggagatgaattgccacattatcataatgaaaatgacaatgaagatcatggtgaccgaaaacctgacgacgacccatattctagcagcgatcccgatccggattttgacagtctggaagattctctacgcaattttgttttgaagaaaagatcactttctaaattgttacctgccaagacttatctatcgaaattattaagcgatataataatgatctccaaatctaatatgaaaatgtatagtgaagaagaaataaaaattaacgaacaactggaaacattgagacctgaaattttgagcgctagagcaaagtgcaatgatttaacaacttccgttgatcaaatggccgagcaaacaattacaatgacttataacaatactaaagaagcacttctcaatgcgttggatgttccgctacacgaatatccgaaatatcagggcctcggtcaaatttatgacttcatattttcaacagaagcgttcatagcaaatcagattgatgagtcaataggatcaagtgaattatttgccaagcaaaagacagatttactggtaaaaaaaatttatgagattggaaaaaatgaacttggtgatgatttcatgtgtgaacgtgttttcagaagcgaactaatgtttagaaaaaggaagcatttgataggaaaaaggcttaaagtatctttgtcaataactgatttgtttgctccaacttggaaaggctttttgtcttacttaagctggcagaaacctgtcacagcaccactgccggatatagaaggacaaactaacgagggtcagattggcctaatgaagtatttgggtttgaaaaattatcctttgacacaatattggtcaagaccatcattgctcttcacatcaaaaattcccactctaacgctgtactttctaggaagtacaaaggttgtggggaacattatattaaacggcattaaattatcgtcgtggagctcactcaagaagttatcagttccggttatagtggtaggttccttgttgggacttacgtatttaattcatgacctcccccgtgccttaccaatgaatctttccattaaatacaaaagaaagttgcaagaactggattacattcatcttaacgctcaaagaacctcaaatgaagtacgtgatgttttgcgtgtacctacacgtgaaattctaaggtcgtgtgaaataattatggataaaaaacaaatcactaagaaagaattggaaaacaaaaaagaaagtaacttgttgtcaatcaaattcttccaatctctatacgaaggaaccgtggctcaaaaattgatggtggaagaaataaatttagacatcgattag

SEQ ID NO: 55 - DNA, 1488 bp, Saccharomyces cerevisiae
atgactacggataacgctaaggcgcaactgacctcgtcttcagggggtaacattattgtggtgtccaacaggcttcccgtgacaatcactaaaaacagcagtacgggacagtacgagtacgcaatgtcgtccggagggctggtcacggcgttggaagggttgaagaagacgtacactttcaagtggttcggatggcctgggctagagattcctgacgatgagaaggatcaggtgaggaaggacttgctggaaaagtttaatgccgtacccatcttcctgagcgatgaaatcgcagacttacactacaacgggttcagtaattctattctatggccgttattccattaccatcctggtgagatcaatttcgacgagaatgcgtggttggcatacaacgaggcaaaccagacgttcaccaacgagattgctaagactatgaaccataacgatttaatctgggtgcatgattaccatttgatgttggttccggaaatgttgagagtcaagattcacgagaagcaactgcaaaacgttaaggtcgggtggttcctgcacacaccattcccttcgagtgaaatttacagaatcttacctgtcagacaagagattttgaagggtgttttgagttgtgatttagtcgggttccacacatacgattatgcaagacatttcttgtcttccgtgcaaagagtgcttaacgtgaacacattgcctaatggggtggaataccagggcagattcgttaacgtaggggccttccctatcggtatcgacgtggacaagttcaccgatgggttgaaaaaggaatccgtacaaaagagaatccaacaattgaaggaaactttcaagggctgcaagatcatagttggtgtcgacaggctggattacatcaaaggtgtgcctcagaagttgcacgccatggaagtgtttctgaacgagcatccagaatggaggggcaaggttgttctggtacaggttgcagtgccaagtcgtggagatgtggaagagtaccaatatttaagatctgtggtcaatgagttggtcggtagaatcaacggtcagttcggtactgtggaattcgtccccatccatttcatgcacaagtctataccatttgaagagctgatttcgttatatgctgtgagcgatgtttgtttggtctcgtccacccgtgatggtatgaacttggtttcctacgaatatattgcttgccaagaagaaaagaaaggttccttaatcctgagtgagttcacaggtgccgcacaatccttgaatggtgctattattgtaaatccttggaacaccgatgatctttctgatgccatcaacgaggccttgactttgcccgatgtaaagaaagaagttaactgggaaaaactttacaaatacatctctaaatacacttctgccttctggggtgaaaatttcgtccatgaattatacagtacatcatcaagctcaacaagctcctctgccaccaaaaactga

SEQ ID NO: 56 - DNA, 762 bp, Saccharomyces cerevisiae
atggaaaatagcatgatgtttatatcgcgttccttgcggagacccgttactgctttgaactgcaatttacaaagcgttagaacagtcatataccttcataagggtccgagaattaatggtttgagacgagatccagagtcgtacttgagaaatcctagcggggtactctttactgaagttaacgcaaaagagtgtcaagacaaagttagatccatactacaattgcccaagtacggaataaatttatcaaacgaactgattttgcagtgtttaacacataagtcgttcgcacacgggtcaaagccatacaacgaaaaactgaatctgctaggagcacagtttttgaaactacagacgtgcattcattcgttaaagaatggttcgccggccgagagttgcgagaacgggcaactttctttgcagttcagcaatctgggaacaaaattcgctaaggaattaacgtccaaaaacacagcatgcacattcgttaaattacataatctagaccctttcatattttggaagatgagagacccaataaaagatggccacataaatggtgaaaccacgatttttgctagtgtcttgaacgcatttattggtgcaattctgtcaacgaatggatctgaaaaagcagctaaatttatccaaggaagtttgctggataaagaagatcttcattccttggttaacattgctaatgaaaatgtagcatctgcaaaggcaaaaatatccgacaaggaaaataaagcatttctgtga

SEQ ID NO: 57 - DNA, 396 bp, Saccharomyces cerevisiae
atgctttatgagctgatcggacttgttcgcataaccaactccaatgcgccaaagttggaagcaaaagaattgtcttccacaatcgggaaactgattattcaaaacagaggtgtggtgagggacattgtacccatgggcataaggtatcttcctaaaataatgaagaaagaccaggaaaaacattttcgagcatatcactttttaatgctgtttgattcatcagctgcggtacagtctgaaattctaagaactttaaagaaagatccccgtgtcataagatcatccatcgttaaagttgatttagataagcagctggatagagcctcatcgttacaccgttctttggggaagaagtctattttagaattagtgaatgaagattatcaatccatttag

SEQ ID NO: 58 - DNA, 1053 bp, Saccharomyces cerevisiae
atgtctcaactgccaacagattttgcttcattgatcaagagattccaatttgttagtgtcctcgattccaaccctcaaaccaaagttatgtccttattgggaaccattgacaacaaggatgctattatcacggctgaaaagacgcactttctatttgacgaaaccgtgagaagaccctctcaagatggccgctccactccagttttatataattgtgaaaacgaatattcttgcatcaacggaattcaggaattaaaggagattacttccaatgatatttactactggggtttgtcagtgataaagcaggatatggagtcgaaccctaccgccaagttgaatttaatttggcctgctactccaattcatatcaagaaatatgaacaacaaaactttcatttagtcagagagactcctgagatgtacaagagaattgttcagccttacatcgaagaaatgtgtaataacggtagacttaaatgggtaaacaacattctttatgaaggcgctgagtcagaaagggtcgtttataaagatttttctgaggagaataaggacgacggcttcctaattttacctgatatgaaatgggatggcatgaacttagactctttatatctagttgcgatcgtctacaggaccgatatcaagacaataagggatttaagatattctgacagacagtggttaatcaacttgaacaacaagattagatccattgttcctggttgttacaactatgctgtccatccggatgagttgagaattttggttcactaccaaccttcttattatcatttccacattcacatcgttaacataaagcatcccggtttaggtaatagtattgccgcgggcaaggccatcttgttggaagacatcattgaaatgttaaattatctaggaccagaaggctacatgaacaagacaatcacctacgctattggtgaaaatcatgatttgtggaaaaggggtctggaagaagaactcactaaacaattggaaagagatggtatcccaaaaatacccaagattgtgaacggttttaaataa

SEQ ID NO: 59 - DNA, 180 bp, Saccharomyces cerevisiae
atggctaagtctaagaaccataccgctcacaaccaaaccagaaaggctcacagaaacggtatcaagaagccaaagacctacaagtacccttctttgaaaggtgttgatccaaagtttagaagaaaccacaagcatgccctacacggcactgctaaggctttggctgctgccaagaaataa

SEQ ID NO: 60 - DNA, 189 bp, Saccharomyces cerevisiae
atgtctgcctggaggaaagctggtatatcatatgctgcatatttgaatgtggccgctcaggctatccgttcttcattgaaaactgaattacaaaccgctagtgttcttaacagatcgcaaacagatgctttttatacccaatataaaaatggcactgcagcttctgaacccactccaataacaaaatag

SEQ ID NO: 61 - DNA, 1977 bp, Saccharomyces cerevisiae
atgttggcccgttccagggtgtgcttgcagacaatcactagacggttggcagactttccagaagccaatgctattaagaaaaaatttctctttaggaaggacacctcgaccatcaaacagttaaaaggactgtctagcggccagaaaatagtactcaatggatggatagagcagaagccaaaaagagttgggaaaaatttgatcttcggacttttaagggactctaacggtgacattatccagttggttgataacaaatcgttgttgaaaggctttactttagaggatgtggttcaagccgtaggtatactctctttgaagaggaagctatcaaatgaggacgcagatgagtacgaagtgcaattggaggatattactgtgttaaatgcatctaataaaaaaccagcgcaaatgcaggattttaaattgtcagccatatacccacctgagttccgctatttgcaattgagaaatcccaaatatcaagattttttaaagaagaggtcatctatctctaaggaaataagaaactccttcaacaattttgattttacggaggtcgagaccccaatgttatttaaagctaccccagaaggcgcaagagagtttctggttccaacaaggacaaagagatccgatggtaaaccatcgttttatgctctagatcagagccctcaacaatacaagcaactcttaatggctagtggtgtcaacaaatattatcaaatggcaaggtgctttagagatgaagatttaagagcagacaggcagcctgagtttacacaggttgatatggaaatggcctttgctaattctgaagatgtcatgaaaatcatagaaaagacagtttctggggtatggagtaaattttccaaaaaacgaggattattgactttagacagtaagggtacattagtgcctgcgaaaaaggaaaacggcacagtatctatctttcgtatgacctacgaacaagccatgacctcatatggtattgacaagccagatttgagagcgccagatttgaagattatcaatttaggcgagttcaatgcctttagtcatttgaacaaaaaatttcccgtttttgaagtaattattctaagaagtgccttttcaaatatggaagagtacaaagaacgatggtcgtttctgacaaataacagtaattacaattatagagttccaatagtgctaccaattgaaaatgacgaacaagctaattcaaattggtttgagaattttcatgcaattgccacgtttgaaaacccacatctaataaccaaatttctgaaactgaaaaaaggtgacattgtatgcggttgtacgagagagccaaaccattccattttcgagaatcctactcccctgggaagattgagacagttggtgctacaaagtgagcatgggaaaaatatctatcatgctgtcaataaggatgttgcctcatggattgtggatttcccgttattttctcccgttataattgaagataagtctggtaaaaaagaaaagcttgcatatccggagtacgaaaaggatagactatgttccacgcatcatccttttactatggtgaagcttaaagactacgaaaaattagaaaagacgccggaaaagtgcttgggtcggcattatgacctcgtagttaacggtgtggaacttggtggtggctcaacaagaattcacgatccaagattacaagactatattttcgaagatatcctcaaaatagataatgcatatgaactatttggccatttactgaatgcttttgatatgggaacaccgccacacgctggatttgctattggttttgatcgtatgtgcgctatgatctgtgaaactgagagtataagggacgtaatcgccttcccaaaaagtattaccggggctgatttggttgtcaagagtccaagtgtgatacctgaaagcattctggaaccttacaatatcaagtatagtaattcaaaaaaatga

SEQ ID NO: 62 - DNA, 348 bp, Saccharomyces cerevisiae
atgatgttcaccttcatattccatattttcaatggttttttccactgttttttcaaaatattctacttcattttccggttctatagagccaatttgtttttcctttggttgtatttccttcatctcgtaatgggaaacatcgtcaaggtcgttacgattcatattcatattagagcttccctcattattccgcccatggcgtctattaccaagcgtcataatgtgcaatatttgatattatataagctacttgagaaagcgatagtttttttttcttacacaaaaaaaaaaaaacataaagcacctataactctcaactttgaagaagcacgaaaggaatatgtttaa

SEQ ID NO: 63 - DNA, 969 bp, Saccharomyces cerevisiae
atgaaagcggaaaggcaaacaggattaagaaatagttttacaaccgtaatcggtcgcaaactaataaacacttttgttccgtccatgatgttaactagcgtagctggtaacgatatttttttccgaggtttattcaaatcgccggtattagcatttcaaagttatcgttatgtttcgattttgggacaattgaaaccgtcagatggttctacaaagtctttcaaaagacttgggcgtggtccttccagcggtttaggtaagacatcagggcgtggtcaaaagggtcaaaaggcgcgtggtaaagtaaaatcgtggtttgaaggtggtcaaactccaatttacaagctatttcctaaaattggatttaccaacgttggcgccaaaccattaaaggaattaaacttgaaacgcattcaatggtttcatgataaaaataggcttcatttgcaaccaggcgaggttttagacatgaataagatgagaaagcttggtctggtaactggtcccataaagtacggagttaaaattctagccagcggaaagttccactacaatctaccgattgctttggaggcttctagagcatccgctaaggctattgcggctattgaaaaagcaggaggtaagtttacagctcgttactacacaccgttgggtctaagggcacatctcaacccccagtggtttcttgagaagaggggaagagtaccactacaggctagacccacgaaaagaagggatattgatttctatagtaaagaagagaaaagaggttacctagtaatggaaaaagataaactcttacaagatatcaaagaagctcaaaataagggttctagacactttttaaaacagaatgtaaaaaagagctcattagaaatagagctagaagaactgtctccagagaaggattgggtgccagttgtgtctaattctaaagtcatgaacatcaaggctttggaccactaa

SEQ ID NO: 64 - DNA, 1116 bp, Saccharomyces cerevisiae
atgtggaatcctattttactagatacttctagtttttcattccaaaaacatgtgtctggtgtgttccttcaagtgcgtaatgccaccaaaagggctgcgggctcgagaacaagtatgaaggattctgcaggaagaaggttaggaccaaaaaaatatgaaggccaggatgtttccacaggtgaaattattatgaggcaaagaggaacaaagttttatcctggagaaaacgttggtatagggaaagatcattccatattcgccttagaacctggtgttgttcgttactatcttgatccttttcatcctaaaagaaaattcattggtgttgccttgaggcgtgatttgaaactgccatctcctcattttgagcctaccgtaaggaggtttggccgttttgagttaacaaataaaagggcagcatataaggaagaaaattctatttctagaaaagactatctcgcaaaacctaatattttgaagcagttagaggtcagggaatctaaaagaaaggagttacaagacaagttgagtaaggttcttcgggatgaacttaagttagatataaaagacatcgaattggcaacgtcatacttaattcgtgtaagggcttccctgaagaatggataccccatagaagatgcaaggttcaatagtagatattatttaaaagaagaagagcgtttgaaggcaaggagagaaagctggacgaatgagaaattgtcggagagtctatccaagattgatgagtgtagtgatcttttgaactcatctacatctttcaataacaagctagagctacaccagtatatttcggaacaagaaaaacaagctttgaaggcaaaactattagaggacctagaaaaatcccaacacttagaaaccaaaaaagacaagaattatattaaggcgcttttcaaggacgcttgtaattttttgactttatctgaagaagtacatttgcgtaggaaatacttaaagtcggtcttccccgaaacagatagtacggttgaaaccaaaagcgggaagaagtcaattgtgtctagacgttttgattacaccaaaaataaagttgaagtcattgctagaagtaggcgggcttttttgagcaagctttga

SEQ ID NO: 65 - DNA, 933 bp, Saccharomyces cerevisiae
atgaatttattacgaatgaatgctttaacaagcaaggctaggtcaatagaacggttaaaacaaacattgaatattttatccattcgtaatcatagacaattcagcaccattcaacaaggcagcaaatataccctcgggtttaagaaatacttgacattattaaatggagaggttggttcttttttccatgatgtccctttagatttgaatgaacatgaaaagaccgtaaatatgattgttgaagtaccaagatggacaactggaaaatttgaaatatccaaagaacttaggttcaatccgatcgttcaagatacgaagaatggcaagcttaggttcgtgaacaatatctttccctatcacggctatatacacaactatggtgctattccccaaacatgggaagatcccacaattgaacataaactggggaagtgtgacgtggctttgaaaggtgacaatgatcctcttgattgttgcgaaatcggttcggatgttttggaaatgggttccattaaaaaggtcaaggtattaggatcactcgctttaattgatgatggagaattggattggaaggtcatagtgatcgacgtaaatgatcctttatcctctaaaatagatgacttggaaaaaattgaggagtactttcctgggattctagatacgactagggagtggtttagaaagtataaagttccagcagggaagccattaaatagtttcgcctttcatgaacagtatcaaaattccaataaaaccatccaaactataaagaaatgtcacaattcgtggaaaaacctcatttcaggatcactccaggagaagtatgacaacctgcctaatacagaaagggctggtaatggtgtcacacttgaagatagtgttaaacccccttctcaaataccgcctgaggtgcaaaaatggtattacgtttaa

SEQ ID NO: 66 - DNA, 834 bp, Saccharomyces cerevisiae
atgggtttacctaaatcggcatataagaagttactcatagattgtccaacgagagtcattaataaaaactgtgcccaacgagtcaaagatgtttctccgctcattacaaacttcgagaaatggtcggataagcgcaagaagctctatttcaaagatgaagaggaaatggtggggcagttccatttggaaaatttcaatttaaaaaataacttatatggtaggcttttagcttctccaatgagagcagaaaaaatctcaaaattaaaatcgtgtagagaattactaattccattgaaagtggtaccatcaactggcaaagatcagcatgctgataaagataaactaaaactggtacctacactagattattccaaatcatacaaaagctcgtacgtattgaattcagcctccattgtacaagacaacttggcggctgcgacgtcatggttcccaatctcggtattgcaaacatcaactccaaagagcctagaagttgattcctctacatttataacggaatacaatgctaacttgcacgcatttataaaagcaagactttcagtgataccgaatgttggaccttcttcgataaatcgtgttttactcatttgtgacaagagaaaaacacccccgattgaaatacaagtagttagtcatggtaaagggctaccaattacacagtctgtctttaaccttggttatctccacgagccaactctagaagctatcgtgagtaaagatgcagttactaatggaatttacctggatgccgacaatgacaaggatctcatcaaacacctttatagtacattattgtttcattcagttaactga

SEQ ID NO: 67 - DNA, 552 bp, Saccharomyces cerevisiae
atgaattctttgatttttggtaaacaattagcatttcacaaaattgtgcctaccactgcaattgggtggttggtaccgctaggaaatccttcactgcagattccaggccaaaaacaactgggatctatccaccgttggttgagagaaaagctacaacaagatcataaggacactgaagataaagattttttctctaataatggtattctactagcagttcctaaaaaaaaagtatcacaccaaaaaaaaaggcaaaaactttacggtccaggtaagaagcaattgaagatgattcaccatttgaataagtgcccatcatgcggccattataagagagccaatacactgtgtatgtattgtgttggacaaataagtcatatatggaaaacgcataccgctaaagaagaaattaagccgagacaagaggaggaactttccgaactagaccaaagagtcctatatcctggtagaagagataccaaatataccaaggatttgaaagataaagataactatttggaacgtcgcgttcggactttaaaaaaggactag

SEQ ID NO: 68 - DNA, 441 bp, Saccharomyces cerevisiae
atgatcagttcgtgtgttactagatgttttggtaggggtaaatgccttccagggcctgccactgcctcgatataccaaacgataagatgtatatccactaattcaaataaagctgctgaggcgccaatatttccaaagctggaagacgtgaagatgcatgagctcataggaaacaacaattttggtaaaaagacctactacgtggagagaagcaggaccggaaatctaccggtgtattccgcttataaaaatggaggtaacaagattatcacggagatcagaaagattgaaggagatgtaattcaactaagaaatgacttgcaggagcaactgcctttcatacccaaaaaatcatggtctgtggtgatgcaatcgaaaaaaattattatcaagggcaatgcagtagaggctgtcaaaagggtattaaccaagaaattttag

SEQ ID NO: 69 - DNA, 816 bp, Saccharomyces cerevisiae
atgtcttttgatattaattggagtacattggaatctgacaatagactgaatgatcttataagaaaacatttaaattcatatttacaaaatacgcaactaccaagctacgtaagtaatcttagggtactagattttgatctgggaaaagttggtcctgctataacactgaaagaaataacagatcccctagatgagttttatgattcaattagagaggaggctgatcaagagacggaggaaaataatgacaacaaggaggacagcgaacatatttgtcctgatcgtactatagctaatcatgaaggacctaaagatgattttgaggcgcctgtggtgatgccatcccccaacgatatacaatttttactagaggtcgagtacaagggtgacttactcgttacgattggtgccgaccttgtgctgaattatcccgtagaaaaatttatgactttgccagtaaaactttctatcagtgatatcggtctccattccctttgcattgttgcatgtttatcaaagcaactatttcttagctttctgtgtgatgttagtgatcctgcactggatgacaaccaaactgttttagatcctaaggggccaattctggcagctactaagccattggagcgaatatccattgtgagaagtatgaagattgaaaccgaaataggggaacaatatcagggacagggctcagtattaagaagtgttggagaactagaacagtttttgtttacgatattcaaagattttttaagaaaggagttagcatggccaagttggattaatctggatttcaacgatggtgatgagtaa

SEQ ID NO: 70 - DNA, 324 bp, Saccharomyces cerevisiae
atgagtagggctagcaagataacgtttgcagcttcctgtctgataacggcagcgacggtagtgggcgtgcattatgtgcaagaaatggaaagggaaactttgcatcagggtccgataaaagatgctaaacgagtcgaagaaaagaggttgagaaagacaaacggagttgcatcattagatcccacaaaagaaaggaaaaggtacttcaatatgagtgaacacgaggaacaaaaagagttgcgaaagaagtatgagaccatgcaaccgcttagtggagaagttgtgaccaaagatggagaggtggttaaagaatctaagaaataa

SEQ ID NO: 71 - DNA, 510 bp, Saccharomyces cerevisiae
atgtggagcaggaacgtcagattgcttggatcatggacaaggtcctacatggtccccgccaccaagagaaaaaccatccccgtgtacccacctgtgcagcgcatagcttcgtcgcagattatgaagcaggtggccctctcagaaatagagtctctggatcccggggccgttaagaggaagctcatcagtaaaaagaacaaggaccgcttgaaggcaggcgacgtggtccggattgtgtacgactcgtccaagtgctcgtacgacacctttgttggctacatcctttccatagaccgcaaacaactggtgcaagacgcctcgttgctgttgcggaaccagatagccaagacggccgtcgagattagagtgccattgttttcgccgctgatcgagagaatcgacttgctaaccccccacgtctcgagcagacaaagaaacaaacactactacatcagaggtacaaggttggatgtcggcgacctcgaggcaggtctaagaagaaagaaatag

SEQ ID NO: 72 - DNA, 444 bp, Saccharomyces cerevisiae
atgctggcacaaacattcaaaaaaccacacagagccgttctagaacaggtatctgggaccacggtcttcatcagaaataagagaacaaagagcaagagctcactgtcacctttggcacaaagggtcgtcacgcagttgagtgtgatgtctgcaagcagaaagcagcccaagttgctgaagctggcgcgtgaagacctgattaaacatcagaccattgagaagtgttggtcaatttatcagcagcaacaacgggagcgcagaaatttacagttggaattacaatataagagcattgagagatctatgaaccttctacaagaactcagccctcgtctgtttgaggctgccaatgcttccgagaagggcaagcgattcccgatggaaatgaaggtgcccactgacttccccccaaatacgttatggcattataacttccgaaaatga

SEQ ID NO: 73 - DNA, 879 bp, Saccharomyces cerevisiae
atgtatgaagtaatacagaaaaggaaaacaaaaataataaacgttttacagagtcctgaactcatgaggctcatagaggacccatcaaatctgggtatttctttacattttccagtaagttcactgctaaaaagtaataagtgcacaccaatgcctaaactttctacgtatagtttggctagtgggggatttaaggattggtgcgcggacatccctctagacgttccaccagagattgatatcatcgatttttactgggatgttattttatgcatggaatctcaattcatattagattacaatgttccgtcaaaaaataaggggaacaatcagaagtctgttgctaagctgttgaaaaataagcttgtaaacgatatgaaaactacgttaaaaagactaatttataatgaaaataccaagcaatataaaaataataatagccacgatggttacaattggagaaaactaggctcgcagtatttcatactgtatcttcccctatttacgcaggaactgatttggtgtaaacttaatgaaaactatttccatgttgtattaccatctttactgaatagtaggaacgttcatgataaccacagtacctatataaataaagattggttacttgcccttttagagctaacttccaacctgaaccaaaacttcaaattcgaatacatgaaattgagattgtatattttaagagatgatttaattaataatggtttggatcttttgaaaaatcttaactgggtcggtgggaaactgattaaaaatgaagatagagaagtcttgttgaactcgaccgatttagctacggattctatttctcatttattaggtgatgaaaactttgttattttagagtttgaatgctaa

SEQ ID NO: 74 - DNA, 801 bp, Saccharomyces cerevisiae
atgcttgttttcaaaaggggtattcacgtagtacctaaactaccaaattctaaggccctactgcaaaatggagtgcctaatatactcagttcttcgggattcaagacggtgtggttcgactaccagcgatatctatgtgataagctaacgctggcgacagcgggacagtctttggaatcatattatccatttcacatattgctgaaaactgcaggaaatccattacagtcaaacatcttcaatttggcgtcctctatccataataaccatttattcgtggagaacattctaccttctgctgttgaacatggcactaactcgaacgccgttgtgaagacagagccttctaggctttttttatccaaaatcaaagattcattcaacggcagcgattgggaagtagtgaaggaggaaatgatttaccgtgctgagaatgaagtcttgggacaaggctggttatttctagttgaaaacaatgagaaaaaactcttcatattaacgagcaataataatggtacgccgtattattttcccaggaaccagtcatttgatttaaacagtgcgatatccatcgatgaatttgctaccttgaaacaaatgaaagaactgataggaaagtctactaagttgaatggtaaggttcaagactggaccatgcctataatctgtgtcaatctgtgggatcatgcttatttacatgattatggcgttggtaaccggtccaaatacgtgaagaatgttctagataacttgaattggtcagtggtaaataataggatattctctggtatatcgaagtaa

SEQ ID NO: 75 - DNA, 348 bp, Saccharomyces cerevisiae
atgggtaatttcagatttcctataaaaaccaagctaccaccagggtttatcaatgctcgcatacttagggataacttcaaaagacaacaatttaaagagaatgaaatccttgttaaatctttgaaattcatcgctagaaatatgaaccttccaacaaaactgaggttggaggctcagttaaaactaaatgcattgccaaactacatgagatctacgcagatcaaaaataggtgcgtggactctggacatgcaagatttgttctaagtgattttaggttgtgtaggtatcaatttagagaaaacgctctgaagggtaatttgccaggtgttaagaagggtatttggtaa

SEQ ID NO: 76 - DNA, 1329 bp, Saccharomyces cerevisiae
atgtcaagaagagtggttatcacaggattgggctgtgtaacgccgttgggaagatcattaagtgagtcatgggggaatctgctctcttccaaaaatggactcacaccaatcacatctttgcccaactataatgaggactacaaactcagagaaaaaagtatcccatcaacgataacagtggggaagattccagagaattttcaaaacgaaaattcagccatcaataaactgctgttcactagccaggatgagagaagaacctcaagctttatcaagctagcactacgtacaacttatgaagcgcttcacaatgctggtctcttgaacccaaatgatataaccatcaatacatctctgtgcaatctggatcactttggttgcctgataggttctggtataggatccattcaagacatataccaaacttctctacaattccataacgacaataaaagaataaatccatatttcgtccctaaaatccttacaaatatggcagctggtaatgtttccatcaagtttaaccttagaggattatcccatagtgtttccacagcatgcgcaacaggtaataactccattggcgatgcattcaattttattcggttaggcatgcaagacatctgtgtcgccggtgcaagtgaaacgagtttgcatccgttaagtttagcaggtttcatcagagcaaagtcgattactacaaacgggatctctagaccctttgatacacaacgttctggattcgtacttggtgaaggatgcggaatgattgtcatggaatcgctagaacatgctcaaaagagaaatgcaaacataatttctgagctcgtgggctatggtttaagcagtgatgcctgccatattacctcccctcctgctgatggaaatggtgccaaaagagcaatagagatggctctaaaaatggctagattagaaccaactgatgttgactacgtcaatgcacatgctacatcaactttactaggcgataaagcagagtgtctggcagtagcctcagcactcttaccaggaagatccaaaagcaagccactgtacatatccagtaacaaaggtgcaattggccatcttttaggtgcacgtggcgccgtagaaagtatatttacaatttgttccttgaaggatgataagatgccgcataccttaaacctggacaatgttctgactctagaaaataacgaggccgataagctacatttcataagagacaaacctattgtgggagctaatccgaagtacgcattatgcaacagcttcggatttggaggagttaacacatctcttctcttcaagaaatgggaagggagttaa

SEQ ID NO: 77 - DNA, 651 bp, Saccharomyces cerevisiae
atggtactggtgctctcgccgcgattggtgaaccagttctgtgacgtgttgaagatcaaagaaggaagaacatccttgaacactacatgcatggactcttttagccttctctcaatggtgctttggagaatttttacagaacaatcaaacttaaaatccaaatcaacgtcattaaaggatatcccaattccagggttctttacaaaaatattcgtgatggcttctagctcgatggaactgaacgtcatagttataggaattgtgaacgatccattattgatgagctggggcatggtaaagctgggtgtatcctgttcgttaatcaataaaagattcgactctatgactgtctgtatccgcaacatagcatccttacatgaaatcttgcaaatccccttggccagagacttgggctgcgtgataatgtctaggtccagaatttctagctgtggtattgtaggaaaatccaccttctgtaccttaattccgcttttcaagatatctaatttctttttagacggtgagttcaaagccgtagataacttttcccgtattcgttcattgaaagagttatcaccaaacacagcttcgttgaatctaaatgacatgacgatgctgaagtttcttgcagttgtgttttttttgttataa

SEQ ID NO: 78 - DNA, 924 bp, Saccharomyces cerevisiae
atgttcaagaggcaattatcgactagtgttcgttatctacagcattatgatgagtcgctattatcaaggtactatcctgaaagtcttttaaaatccataaaacttgctcaacaaacgataccagaagatactaaattcagagtatctcgtaacgtggaattcgcaccaccatatttggatgattttaccaaaatacatcctttttgggattacaaaccaggcatgccccatctccacgctcaagaggagaataataacttcagcatctttagatgggaccaggtacaacaaccattaccaggcgaaggtaacattctgcctccaggagtcagcttaccaaacgatggtggtcggaaatcgaaaagcgccgatgtagctgcagggctacacaagcaaaccggtgtagatccggattatatcactagaaagttgactatgaagccgctggtgatgaaaagagtgtcaaatcagactgggaagggtaaaattgcgtctttctatgccttggttgtcgttggtgacaaaaacggtatggtaggtttgggagaaggtaaatctcgtgaagaaatgtccaaagcgatctttaaagctcattgggatgcggtaaggaacctgaaggaaatacctaggtatgaaaacagaactatttatggtgatatagattttaggtatcatggtgtgaaactacatttaagaagtgcaaaaccagggtttggattacgtgtaaaccatgtaatctttgagatttgtgaatgtgcaggtatcaaagatctgagtgggaaagtatataaatccagaaacgatatgaatatagctaaaggtaccatcgaggctttcacgaaagctcaaaagacattggatgaggttgccctgggtagaggcaaaaagcttgttgatgtcaggaaagtttactattcaagctga

SEQ ID NO: 79 - DNA, 441 bp, Saccharomyces cerevisiae
atgaaaggctcacccatttctcaatttagtaagacctctattaatgctcttaccaggccttggaagaaatacagagatggtgaattattttatgggttatcaaaagttgggaataaaagagtgccgttgaccacgaagcaaggtaacaagacgatgtacaaagggacaagagcttcagggattggtagacatacaaaattcggtggttatgtgataaactggaagaaagtcagaacctatgttaccccagatatggttaatttcgaattaaagccctacgttaacgcaaatgtaccacctctaaaacacgaatttaaaggatttagcggcggcccattagatcctcgtttacagttattaaagataaaagaatacatagtaaacgggagggtacaaagcgaaggggccaccgacacttcatgttataaggagcgtggataa

SEQ ID NO: 80 - DNA, 2460 bp, Saccharomyces cerevisiae
atgtggaaatggaatgtgcgcagatgggcgggcgcaagagtaaacattagcaagaaccgattaagtgtaattaacgttggaagtagatacttatcgaccgcaagaagtccactgtctaaggttagaaatataggaattatagcccatatcgatgcaggtaaaactaccacaacagagaggatgctttattatgcaggaatctcaaagcatattggagacgtcgacactggcgatacgataactgattttttagagcaggagagatcccgaggtatcacaattcaaagcgctgcgatttcattcccatggcgaaatacttttgcaataaatcttattgatacgccgggacatatagattttacttttgaagtgattagggctttgaaagttattgattcatgcgtggtcattttggacgctgttgccggcgtagaggcacagacagagaaggtttggaagcaaagtaaatcgaagcccaaaatctgctttatcaacaaaatggatcgtatgggggcgagtttcaaccatacagtcaatgacttgataaataaatttatgagaggaacaactaccaaaccggtgttggtaaacattccttactatcgaaaacaaccgactagcaatgattatgttttccaaggtgttattgacgtcgtaaatgggaagcgattaacctggaatccagaaaatcctgatgaaattatcgtggatgagttggatggtacttctcttgagcagtgtaatcgttgtagggaatcgatgatagagactttgactgaatacgacgaagatttagtccagcacttcttagaggaggcagaaggcgattactccaaggtttccgcacagtttttgaacgcttcaataagaaaactgactatgaagaatatgattgtacctgtcttatgcggtgcgtcttttaaaaatattggcgtgcagcctctattagatgctatagttaattatctcccttcgcccattgaagccgaactaccagaactaaatgataaaaccgttccaatgaaatatgaccccaaagttggatgtttagtaaacaacaacaaaaatctttgcattgctcttgcattcaaagtgattacggatccaatcaggggtaaacaaatattcattagaatttattcaggtacactaaacagtggcaataccgtttataattctactactggggaaaaatttaaattgggtaaattactgataccccatgcgggaacatcacaacctgtaaatattttaactgcgggccaaatcggattgcttactggctctacagtcgaaaacaacatatctactggagatacactaataacgcattcgtcgaagaaggatgggttgaaatcacttgataaaaaaaaggagttgacgttaaaaatcaattctatcttcattcccccaccagtttttggtgtttccatcgaaccaaggactttaagtaataaaaaatcgatggaagaagctttaaacactttaattactgaagatcccagtctttcaatttctcaaaatgatgagactggccaaacggttttaaacggtatgggtgaattacaccttgaaattgccaaggatcgattggttaatgacctaaaagcagatgttgaatttggtcagttgatggtgtcctacaaagaaacaattaattcagaaacaaatatagaaacgtatgagagtgatgatggatatagattctccttatctctgctaccaaattccgatgcccttcctaactgccttgcatatccattaggagtcaacgagaattttctgataatggaaaaaaatggtaattgggataaagaatggaaataccaagtttctttcgaatcaattctaaattctattattgcaagttgcattgtgggacttcaaagaggcggaaaaatagctaactttcccttatatgcatgctccatcaaaatcaatagcgattggtcagtcccacctgatattgaaactccacaagaaatcttaaaaattactagaaacttaatttttaaagctctcaatgacttaaaacctgaaaaatacaaccttctggaacccatcatgaatcttgatctaacaatccctcaatctgatgttggtacggtgttacaagatctaacaggagcaagaaaggctcaaattctgtctattgaagacgaatcaagcgtgtcaaattctggcgcatccacttgtaattctccagagaacagcaataggatatatattccctctgatgctgttacaaccctacacgcaaccaaagataaaaaaaacactcaagagaccagttctaatgtaaaaaaaattataaaagcgaaagtgccactaagggaaattaccacctacaccaataagctaaggagcttatcgcaagggaggggtgagttcaatattgaatattccgatatggagaaagttaccaacgatcgcctacaatcaatacttcacgacttgtag

SEQ ID NO: 81 - DNA, 1731 bp, Saccharomyces cerevisiae
atgaatgtgctgttaaaaagacgcagtttaacgtttgcaccgagatggctgtggtgcaagtgtcgaagtagcaggagcaggccgtattctctggcacacgcggtcgacacaagtaagatggaggcaaccaggaggaacggtcagattgtgaaggacctgggtagatactatccgtcaatgtcagaatcagcactccacgatctttgccaggagtataaagaagtcactatcgcagactttaatgagagattcttgggcaacccagccacattacaccatgaggataatcctaatttgttacttagtattaatggcaggattaaaagcattaggttttcaggccagaagattgtttttatagatctttacaacggcagtagcggattgaagaatgatacgcaactgcagttgatcgtaaactacaataaaattggtgggagtagtgaggataaggcgaatttctcagaatatatgaactttttgaagaaaggagattatattaaggcgctcggttatcccgggttttcccaatccagagtcaagatgctgtccttgatatgtaataagctacctatagtattgtctgtttcgcaactgccgttaccttccagactgaatgatgaaaccaagatcaaatctaatagagttgttgactaccaattaaacggcactcagacactgctagtaagggcgcgtatcatcaaacttttgaggaagtttctcgacgacagaaacttcgttgaggttgaaacgcctatactgtcatcaaagtccaacggtgccatggcgaagccctttataacgtcgtccaaggacttcgaccatttggagttgcgtattgcaccagaattatggctgaagaggcttataattagtggtttacaaaaagtatacgaaataggaaaggtctttcgaaacgaaggcattgattccacgcataatgctgagttttccactttagaattctatgaaacttacatgtcaatggacgatatagtcacaaggactgaagatttgttcaaatttttgattactaacttgcaaaagttctttcaagatacccgtctgcctgtccctaagacgtttagtgaattacatctggcactatccgagaataattggaagtttagaaaagtcgaattcctgcccactttgaacaaagagcttggcattgatctaatgaactctggattggatatcaataagccgagcgaacttttgaaggctttaccgaaagacattgcgaagaagtattttccttctgcagataacactgggcaattatcttcgctgcaaatcctaaataaactatcggacgtgttcctcgagcagcgccattgtcaatcaaccttgcctacggtaatataccatcaacctgcaatattatcgcccttagccaagacggatccgcaaaataagcaagtcacgaagagattcgaagtcttcatcaaaggtaaggaatacatcaatgcttacgaagaggaaaattgcccccaattacaactgcagaagtttctacaacaaaagcagattaacgaattgacaggaaacaaaactgagaccttatcgcctgttattgactaccagtatgttgagacgatgaaatacggtatgcctccagtaggtggctttggccttggcatagatagattgtgcatgctattttgcgacaagaagaggatagaagaggttctgccctttggctgtgtggatgatgtaaacaggcagtaa

SEQ ID NO: 82 - DNA, 1239 bp, Saccharomyces cerevisiae
atgacttccctgactaatgccgtgttcaaacgatacctggcagtgactcctagcgctcaccaagctcttaaaacgagaattaagaaaaagtcgtcatctttcgataagtttttcccacagcaaagcaatagtagaaaaaaacaatgggaaacgttgaatgaagacaaagcgtcgtggtttaaaaggaaatacgctcacgtacatgctagggaacaggaccgtgcagcggatccatatggcaagaaaaaggcccacgtcgagaagctaaaggagatcaagaatcaggcgaaattaaaccaaaagagccataaatccaagttccagaataaggacattgctctgaaacttatgaatgataatcctatcttcgaatatgtctacggcaccaacagcgtttatgcagcgttgctgaacccgagtagaaattgccattctcggttgctgtaccatggcactattcctagtaaatttcttcagatagtcgatgaactgaaggttacgacagagctggtggacaagcacagattaaacttgttgaccaattacggggtgcacaataacattgctcttgagacaaaacctttgcagcctgtggagattgcgtacctaggcgacatggacaccagttcagcagcattaagcattcacgagctgggcttcaataacgaaaatatacctcatgaactgccttatggtacgaaaacagacgctaagaagtttcctctcgggttatatctggatgaaataactgaccctcacaatatcggtgccattattagaagcgcttacttcctgggggttgacttcattgtgatgtcgagaagaaactgctctcctttgacgcctgtggtgtccaagacaagcagtggggccctcgaacttctgcccatattttacgttgataaaccgttagagttttttactaaatcacaagaaatgggtggctggacgtttattacaagccacttagctaatgccacgtcagaaaaatacactgttggaaaaactattagcatgcacgatttgaacggattatgtaacgagctgcctgttgttctagtggtggggaacgaaagccagggcgtaagaacgaatttgaagatgaggagtgacttcttcgtcgaaattccctttggtggcattgagaaaggcaatcgggccccagaacccattgttgactcactaaacgtaagcgtggctactgcattgttaatagataatattctaacttgtaaataa

SEQ ID NO: 83 - DNA, 2910 bp, Saccharomyces cerevisiae
atggtagttaggcggaaagtacatgttcttctaattgcaaggagctttcattcctacacaccttgcttcagagtaactaccagaggcaaacgacagcgaagtaagagtaagcagcaagcaaaagtagaactagatcacacacgagagctagataatgatcaagctacagagacagttgtagaccgttcggttggaccggagaaagatatagaatcaataaacaaagactttctgcaacgaacaaaaggattagagcctgatattgaattaaaacaactgcctcaaattaaacaggaatttaaccaacgttataaagataggtatgttaaaccttccgaagattggtacgtaaactcctggaggtctttaacaaaaccaaaaattccgctttacaagttaataaattcagattttcaattaataacaaaactgaaagctccaaacccaatggaatttcaacctgttcagctaatggaaagtcccttaaatgttggtgactttgttcttttaaaaatgagacccaatgaattggcgatgtgtgtaagtcttccaagtagcactatggatcctcgatacacttttgttactattgatggaactatgtgctttgcgaccaaaaaccgggtgttattgaggattcctcacaaattacctgctggaatccattcacttatacagcctgagagccatcacaaacatttgccaattggaaccgttaagaacttttctaaccaaacgaatattttgcctattgtagcaaggcagctaataacaagtaggtatccagcccaaatttcaaagttggcctggaaagatttgcccattactaccaaaaaattacagttactccacagatcattacaaaattatatgggtccatggcagattcctttttttaccctagtcggattagttcagaaattggatttaaataaagccttggacgataaaaatggaataaattacttaactagtttagttaataactatcacactgtaaacgatataccaataaattcacctacctttgtttccacgtactgggcgattatgcaacaacaggaatcgaacctttggggggaaatccatctgaatacagcgttgctgtcgcctatatcagtaacgattattcccttaaaatcacaacatttatattatgcgcaggtgatagagaagctagaagccaacagttatagagaggtcaataagtttgtaaaattggtgaatgaaaggaaatatcgcgatatatcagctttatatccttctgtgatccaattattgaaagattttgctgctgggaattttcacaataacggaattatagtagctttgatctcaaaaatattcagaaagatagaacgctataaggattgtgacatcacaagagatatatgtcaagatttaatcaatgagataacacccaactcaatacccaatccattgttacttaacatggatttagcattacctgcttcctcgaagttagtgaaatggcaacaaaaactctacgacttaactaatatagaagaattgcaatggaaaaaatccggcactgatgacgataggtacgattttggcgatcttagggttttttgtatagactctgaaactgcgcatgagattgatgacggcgtgtcggtgaaaaactatggtagagatggactgtacactttatatattcatattgcggatccaacttctatgtttccggaaagcactaatgttgatattgagggtataagtacagatatcctaaatgttgccttgaaaagatcatttaccacatatttaccagatacggttgttcctatgttacctcaatctatttgtcacttatcagatttggggaaacaaggacaaaggacgaaaactatatccttctctgttgatgttaaaatcacttctaaatgcagtggaaaatcaattgaaattatgtatgacagttttaaaatccggaaaggcattgtgtccaacttcccaaaggccacttatgaagatgttgacaggatactcggtacacccaatagcgaagcatcacctgtcaaaaaggatttggaaagtttgtccatgatttccaaattattgagagaacaacgtataaaaaacagtaatgctgttattttcggggagggctttaataagggcctagtgatgctaaacgccgattcagaaggagaactgactgaggtaacattttcggatcaagaggagacgctgtctaccattttggtttcagaaatgatgatcttggcgaatactttaacgggtagatattttgcggagaacaaaatcggtggtgtgttcagatgttataaacagttaccactgggcgaagtagcacaacaacagtatgattctatgataacaagtacaaaaaaaggaatttttcccaagctaaaagatatcgttaagctttcttcattgttgaactcgagtttttatacaggtcgaccttttagacatgaaatgattggagctaaacaatatttgacagtaacatcccctcttcgtcgttttccagacttgataaatcatctacaaattcaccgtcatttgcagaaaaagcctctatgttttaatcaaacacaaatcgacagtttgatctggccaattcaatctcgagcggatattttaaaaagggctagccgtaattcgtcgacgtactggacactgaattatttaaaaaagctaaccaaactagagccagaaaggactttcgatgtaatggtaacatcagtaccgcagaacggatttactggatgtgtttttccagacttatcgttcgctagaggaacattaaaattgcatccaagttctatgcattatccgatgattggagatatcgtgaagaattgtaaaatttcaaaaattgactgtttagaaggaatgttagagttggaaaagctataa

SEQ ID NO: 84 - DNA, 924 bp, Saccharomyces cerevisiae
atgttgatttcttcacggatcgttcgaagctcgctggtaaatgtcccgctaagattatctaggtgttttacgcaagctcacagagcatgcaaagaagaagaagttaatagtcctttatcatccgcagctgaacagccagagcagaagtatacgcattttggttcgaagactgtattgaagtctaccaagcagaagttagttggtgatgtcttttcttccgtggccaatcggtatgacttgatgaatgatgttatgtcattaggaattcatagattgtggaaggaccattttatcaataaactagatgcgggaaaaaggccaaactctacgactcctttgaacttcatagatgtggctgggggatccggtgatattgctttcggattactagaccatgctgagtcgaaatttggtgacactgagtctacaatggatattgtagatatcaaccctgacatgcttaaagaaggtgagaagagagccatggaacaaggaaaatatttcaaggatcctcgtgtgagatttttggtttctaatggtgagaaactagaggagattgattctgattccaaggacatctacacagtctccttcggtatcagaaatttcaccgatattcaaaagggtttaaacactgcttatagagttttgaaaccgggcggtattttttattgtctagaattttccaaaattgagaatcccctaatggactttgcttaccaacagtgggctaaggtcctacctgtaatgggctcgatgattgctaatgactacgactcttaccagtatttggtggagtctatcgaaagatttcctgaccaagaaacgttcaaatccatgattgagaaggcaggattcaaatctgctggctacgaaagtttaacttttggtatatgtgccatccattggggcattaaagtttaa

SEQ ID NO: 85 - DNA, 960 bp, Saccharomyces cerevisiae
atgtggaagagatcgttccactcccaaggagggcccctgcgtgccagaacaaagtttaccaaaccaaagccaaaacaaccggtgcttcccaaagacaaaataagaccgcccactcaattgactcatcactccaacaacctgaggattaccgaaccaatcccaccaacaacctcaaacctgcgctgtcccgacgaccacccgctgtggcagttcttctccaacaagaaattcatcaggtccgccgacgacttgccgccttccagccatatcagaccctggagcatcccagagttgaggcacaagtcctttaacgacttgcattccctttggtacaactgcctcagggagcaaaatgtgcttgctcgcgaaaaccatctcttgaaaaacattgttggctccacgcatgacgaattcagtgagctctctaactctatcagaaccaccatgtggcaaataagacatgtacttaacgaaagagagctggcctatagcgcttcacgcgaatttttacaggatgaatcggagaggaaaaagttcttggatactttggccaacgattacttcttgaacaaggatatccctgatgacgaagtcgcctccatgctcacacgattccagttggccattttcggcatatcggaaaccatccaggataatacagtcgacattaacttcatcgacgggatcaagttcttggcaaacttaaaactacagcggttcaaggactccaatgacctcatatccgaaatatcacaagaaccgatcacagacgtgggcgagtcgttcatcttattcacttccgacttcgaacctcatgccgttcaagaggcttgtgtcgccatcaaagacctgagaaagtccccagataataaagtccctaaattagacgaactccccaccgtgagaaaatacttgaaacagctaattcatgcaagctccgtggagcaagcaactgcatga

SEQ ID NO: 86 - DNA, 432 bp, Saccharomyces cerevisiae
atggtcgttcacatcttgggtaaaggtttcaaaggtaaagaggtcattaaaatcgcactggcctctaaattttacggcataggtaaaacaacagccgaaaagatatgctcgaagttagggttctacccttggatgaggatgcaccaactatcagaaccccaaatcatgagtatagctagtgaactgtcgacaatgacgattgaaggagacgctagagcaatcgtaaaagacaatatagctctaaaaagaaaaattgggagttatagcggtatgagacatacattgcatctgccagttaggggccagcatacaagaaacaacgcgaaaactgcaaggaagcttaataaaatcgatagacgtggaattcacacattttctcaagcaaaagtacaacataatccgtccctttggtcttgtatctttggcaagtga

SEQ ID NO: 87 - DNA, 417 bp, Saccharomyces cerevisiae
atgcaacctatcatcaagggagcagtaagctccacatttaaacgtgctctatacaatttcggtatcaaagaaaagaagagtgtcaatattgaaatggggagaacgcaacaaacaaagaaaatcgatcagtctttgtccaaaaaacttcccaaaggtactatctacgatccatttgacttctctatgggacgaatccatttagatagaaaatatcaggcaaacaagaactcaaacaggaacgatataatgaagtccggtgcaaatccactagaattttatgcaagaccaagaatactttcaagatatgtgacctccacgggaagaatccaacatagggatatcacaggcttatcggcaaagaatcaaagaagactatcgaaagccattagacggtgccaggcaataggattaatgtga

SEQ ID NO: 88 - DNA, 2313 bp, Saccharomyces cerevisiae
atgcttcgcacgataatattgaaagccgggtccaatgcctccataccgtcaccttcccgccaaaataagttactcaggttcttcgccacagccggcgcagtctctaggaccagtccaggaagcattaagaagattttcgacgacaattcatattggagaaatattaatggtcaggatgctaataatagcaagatctcacaatatctttttaaaaaaaataaaaccggacttttcaagaacccttatttgacttctccagatggtttgcgtaagtttagccaggtttctttgcagcaagcacaagaacttctcgacaaaatgaggaatgattttagcgagagtggtaaattaacctatattatgaacctggacagattaagcgatacgctatgtcgagttattgatttgtgcgagtttattaggtcaacacatccagatgatgcatttgttagggcagcacaagattgccatgaacaaatgtttgaattcatgaatgtcttgaacactgatgtttccttatgtaacatactaaagtcggttttaaacaatccagaagtgtcttcgaagttaagcgcagaagaacttaaagttggtaaaatattattggatgattttgaaaagtcaggaatctatatgaatccagatgttagagaaaagtttatccagttatctcaggaaatcagtttagtaggtcaagaattcatcaaccatacagactatcctggttcaaattctgtgaagataccatgtaaagatctggataatagtaaagtgagtacatttctattgaagcaattaaataaagatgtaaaagggcaaaattataaagtacctacatttgggtatgcagcttatgcattattaaaaagttgtgaaaatgagatggtaagaaaaaagttgtggaccgctcttcacagttgttctgacaaacaggttaaaagattgagtcacttaatcaaactaagggcaatcttggctaatttaatgcacaaaacaagttacgcagagtatcaattggaaggtaaatggcaagatcgaagatgtcaagattttattttgacgttaatgaacaatactatagagaagacagcaaatgaattgaaatttatagctgaactcaaggccaaagatcttaagaagccgttgactacaaacacggacgaaatattgaagctcgtgagaccatgggatagggattactatactggcaaatatttccagctcaacccgtcaaactctcccaatgccaaagaaataagctattattttacattaggaaatgtcattcagggcttgtcagatttgtttcagcaaatatatggtattagattagagccagcaattactgatgagggagaaacatggtccccagacgtgagaagattgaatgtgatatctgaagaggaaggaatcatcggcataatttattgtgatttattcgaacgaaatggcaagacttcaaatccggctcatttcacagtttgttgctctaggcaaatatatcccagtgaaactgatttctcaaccatccaagtcggtgagaatccagacggtacctactttcaattacctgttatttctttggtgtgtaatttttctccaatactaatcgcttctaaaaaaagtctttgttttttgcagcttagtgaagttgaaacgctcttccatgaaatgggacatgcaatgcactcaatgttagggagaactcatatgcaaaacataagtggtacaagatgtgctactgattttgtagagttaccaagtatcctgatggagcacttcgctaaggatatacgaattctgacaaagattggcaaacattacgggactggagaaacaattcaggctgatatgttacagcgcttcatgaaaagcactaacttccttcaaaattgtgaaacatactctcaagcaaagatggctatgctggatcaatcatttcatgatgaaaaaatcatttctgatattgataactttgacgttgtggaaaactatcaagcactagaacgacgtttaaaggtcctagtggacgatcagagtaattggtgtggaagattcggccatttatttggatcggggcaacttattacagctacttattttgataggacgatagcttctaaaatctggtacgcccttttcgaggatgatccgtacagtcgaaaaaatggtgataaatttaaaaagcatttactaaaatggggagggctaaaagacccttggaaatgtatcgctgatgttttagaatgccccatgctagagaaaggcggtagcgatgcgatggaatttatagcacagtctcacaagtcttag

SEQ ID NO: 89 - DNA, 1731 bp, Saccharomyces cerevisiae
atgttgaaatatcgtacattgtctcgatcatgtcatatttttcacccaaaatctctttcaaacaataccctgaaatctgaaactactcaggaactacttcaaactgtgggattcgtaagacgctcgcaggttgggttgttccaatggctgccacttggactcagatcacttaataaagtatcaaatgctatacggaaccgaatggatagcgatggtggagccatagaagtttcattaagtgctatctcgtcaaaagcattatggcaagcgacggatcgctggaacaacagtgaattatttaaattgaaggattctaaagggaagcagtactgcttgacagcaacatgtgaggaggacattacagatttaatgaaaaattacattgctagttataaagatatgcctatcacaatataccaaatgacaagaaaatatagggacgaaatcagacccaggggtggtatcttacgtggacgagaatttttaatgaaggatgcgtattcgtttgctagtaatgaagaggatgcgtttgccagttttcaaaagctagatgatacttacaataagatattcaaagatctgaaaatcccctttgttagtgcgtgggcagatagcggtgacattggcggtgagtttagcaaagaatttcacctgattcatgaatccggtgaggataccttgatgagttgcaaacattgcggcgatatatcaactttggatatgtcccagtcatatcctgaaaaagatggtcaatattcaggtgatgtcgattgtaagtatgctttgactaaggatcactcaacattgatatgtttctactatccaaaggatagacaattgaattggaatttagcattaaatgctatggataaagacatcgatttgacgttgaggaacaaaccaaatgatcacgtcttacaagtctacgaaaaagataacgaagatattatgttcagtaaaatattaagagttatggactgtagattgaattcaaaatccaactttcctgattttccactaaagaaatacctgaaaaataattttggtcaaatcagtgacgtttctatagtggatgcacaggaaaacgaaatctgtggcaaatgtgaggaaggaaggttagaacctctcaaaagtattgaggtaggccacatatttttgctgggtaataaatactcgaagccgttaaatgtcaagtttgtcgacaaagaaaacaaaaacgagacatttgtccatatgggttgctacggcattggagtaagcaggctagttggagcaattgctgaactagggcgtgattccaatggatttagatggcccgctataatggctccatacaaagtatcgatttgtactggaccaaataatcctgaaaactcacagaggttacaggatgttaaatcagagctgctgaatgatcctaccatgcaaaatttacagaatgacatccttgaccagtttaatgaaaaactgggtataggtgctagaataaaattatcgcatgccatgggtataccattatgtgttatcgtgggctcaaaaagttggcccaatgtggaaattgaagttcgtggcataagatggggagaaaaggacctgtggagaaagcagtttgaaaaaagatgtagcgaactgcaatggaagtgtaccaaaaatgaacatggaattgagaaacatactgttccaatccagcatttagcggaagtcattggcgtattgctaaaggatatgtag

SEQ ID NO: 90 - DNA, 1209 bp, Saccharomyces cerevisiae
atgtttcaacagctgtcggcatccattaggcacaatgcacacataatttttttatgcataagttggtattttatttcatcgttggcatctcaggtaacgaagcaggtactaacggtttgcccattaccactttttttgggtgagtttcagttcatttatactgctgtgctagcatggtttacgtgttatattgcatatagtttcccaggattctatcgcatatttcctaatggtacttttccggaatattatatcgacgacagagaaactagccgtgcagctaggaaagaatcaaaactatcctccctcattataccgccctctaaaccaatcctgcaaacagttctacctttaggtcttttccaatttgtagggaaatattttgggcatacagctacctctttggtgcctgtttccacggttgcaagtataaaaacgctatcccccatgtttatccttttgctccaaaagattttgaaaatatccactttgaaaattactttaactcttatctttagtttatgcactttagttcttggagtttggattatagttcaagaagataatcgttcaccggcctcttccaatgagctgagagaattctctaaatacggcgtaatatgcgctatgatatcaatgttcatttttgtcttgcaaaatatatatggcaagaccgtgttcacgtacagatctcaaacagatgaatctcaaagtaattctggattctcacggcaagaatcacctttacccttatatgaaaagttggatgagaaacttgttgcgaaaaagaaacctaaatcatatgacaaattgactttaatgatttatatttcccttgttggattttgtttatcgtttggctggtttatcactctagaatttcctgtactatttaggtatttttttcaaattaacagctcttctacagtaataaaagcttttccagtctctttatttttactgaacgggacattccacttcattcaggcaatgattacatttcatttactgggtgaggtctcaactttaacatattcaatagcgaatctgatgaagcgctttgccattattgcggttagttgggtctttattggaagaagaattacttggctgcaagtgttcggtttggttctgaacacattagggttattcctttatgaaagatgcacatcacaatcgaaaataaaagcgaaaatacgtcctgagtag

SEQ ID NO: 91 - DNA, 1281 bp, Saccharomyces cerevisiae
atgactgatagtgagaatgaatccaccgaaacggattcgttaatgacgtttgacgattatataagcaaagagctacctgaacatttacagagactaatcatggagaatttgaagggttctactactaatgacttaaagcaaacttcaaacaactcagagtttaatgtcagtaaaaacgggagcttcaaaggtctcgatgatgcaattcaagctttgcaaatgcaaagcgtgttgcatccttcttcgttaggatcgttagcaacgtcctccaaattttctggatggtcgtttgctcaagggttttttgtaggacagctaagcatagtgttgttgttcatctttttcctaaagttctttatattcagtgatgagccatctaaaagtaagaatccgaaacctgcagcctcccgtcacagatcaaaatttaaagaatatccctttatatctcgcgaattcctgacttctcttgttaggaagggtgctaaacaacactacgagctcaatgaagaggcagaaaatgaacatcttcaagaactagctcttattttagagaaaacctattataatgtcgacgtgcaccctgcagagtcattggactggttcaacgttttagttgcccaaataatacagcaattccgcagtgaggcttggcacagggacaatatccttcattccttgaatgattttattggaagaaaatcacccgatctgcctgaatatttggataccataaaaataactgaactggatacaggtgatgatttccccattttctcgaattgcagaatacaatattcgccaaattcaggaaataaaaagctagaggctaaaattgatatagatttaaatgaccacttaactttaggagtagaaacaaaactattacttaactatccaaagcctggtattgccgcactccccataaatctagtagtgtcaattgtgaggtttcaggcgtgtttgaccgtatctttaactaatgcagaggagtttgcttctacttcgaacggtagcagtagtgaaaacggtatggagggcaattcaggatactttttgatgttttctttttctcctgaatatagaatggaatttgaaatcaagtcgctaattggctcacggtctaaacttgaaaatattcccaagatcggcagtgtcattgaataccaaataaaaaaatggttcgttgaacgatgcgttgaaccaagattccaatttgtcaggttaccaagtatgtggccacgtagtaaaaatacgagagaagaaaagcctacagagttataa

SEQ ID NO: 92 - DNA, 534 bp, Saccharomyces cerevisiae
atgttgaagagcacgctgaggctttcaagaatctctctcagaagaggtttcacaacgatcgactgtttacgccaacaaaattcggatatcgataaaatcatactaaatccaatcaaattagctcagggaagcaacagcgatcgtggccaaacctctaaaagcaaaactgataatgcagatattttatcaatggaaattccagtagatatgatgcaatctgctgggagaataaacaagagggagcttctatccgaggcggaaattgctagaagtagcgtggagaatgcacaaatgagattcaattctggaaaatctataatcgtgaataagaacaaccctgcagaatcatttaagagattaaacaggatcatgtttgagaacaatattcccggagataaaagaagtcaacggttttacatgaagccggggaaagtggctgaattgaagagatctcaaaggcataggaaggaattcatgatgggcttcaagaggttgattgaaattgttaaagatgccaagaggaaaggatactaa

SEQ ID NO: 93 - DNA, 1647 bp, Saccharomyces cerevisiae
atgcataaaggaaaaaaaaattaccctaatttgattacttcgtttcggatgaatttgaaaaaaatcatattgaaccatgaccgatttagccatccagaaagatggaaaacaaacgcactcttaaggttcaccttcgtgtatataaaatttctcttcgacttgatgataatcaaaaatccattaaggatggttggaaaaacttatcgagacgctgttactgcgctaaactctttgcagtccaattatgccaacatcatggctatccgtcaaactggtgatcgtaaaaacactatgacattattggaaatgcatgaatggtctcgaagaattggttactctgcatcggatttcaataaattaaatattgttcatatcactggaacaaaaggtaaaggttctactgccgcatttacttcatcaattctaggacaatataaagaacaattaccacgtatcgggctatatacgtctccacatcttaagtcagtgagagaaagaataagaataaatggggagccaatttctgaagaaaaatttgcgaagtacttctttgaggtttgggaccgtctagatagtacaacctcttctttggataaattcccgcacatgatcccgggaagtaagcctggctatttcaagttcctcacattgctttcatttcacacttttatacaagaggactgcaaaagctgtgtctacgaagtcggcgtcggaggtgaattggatagcactaatataattgaaaagccaattgtctgcggtgttacactattaggaatagaccatactttcatgcttggtgatactattgaagaaattgcttggaataagggaggaattttcaaatctggagcaccagcattcaccgttgagaaacaacctcctcagggattaacaatattgaaagaaagagctgaggagcgcaaaacaacgctaacagaagtacccccatttaaacagttggaaaatgtcaagcttggaattgctggcgagtttcaaaaaagtaacgcctcccttgccgttatgctggcttctgagatcttgcatacgtccaatatattggaggagaagatcaaatgcagttcgaatgcatcgattccagaaaaattcataatcggtctacaaaatactaagtgggaaggcagatgtcaagtactagaaaagggaaaaaacgtttggtacattgatggtgctcacaccaaagatagtatggtagctgcatcaacatggttcagagatatggttcgattgtcgaagagaaagaagatcttgctcttcaaccaacaaagcagagatgccaacgctcttgtgaataacctgtactcatctgtttcgccagaaataacgttcgatgatgtgatatttactactaatgtcacttggaaatcaggctcatacagcgctgacctcgtttccatgaacacttctcaagaagatgtagaaaaattgaaagtccaggagtcattagtcaagaattggaacaaaatagacgataatcgtgctaaaacgcatgtaacggctagtatagaggaagcaaatgaattgattgaaacgctatacgatgaacctgctgatatatttgtaactggttcattacatcttgttggcggattattggtcgtttttgatagaatagatgtaaagtaa

SEQ ID NO: 94 - DNA, 375 bp, Saccharomyces cerevisiae
atgttccctatcgcttccagaagaatactgctcaatgcttcagttctgccattgagactgtgcaatagaaatttcactaccacaagaatatcctacaacgtcatacaagatttgtatttgagggaactaaaagacaccaaactggctccaagtaccttgcaagatgctgaaggtaatgttaagccttggaacccaccacaaaaaccaaatctaccagaattggaacttcaaggcccagaggctttaaaggcttacaccgagcaaaatgtagaaactgctcatgttgctaaagagtctgaagagggtgagtcagagccaattgaagaggattggctagttttggatgatgctgaggaaaccaaagaaagtcattga

SEQ ID NO: 95 - DNA, 1479 bp, Saccharomyces cerevisiae
atgtttcatgctttcaccttccttaaaggtggtagattttactcttcactaacagttaaatcattgtacgagcaggtacaccatactagccatgatcccatttcaattaatggatggatcaaatccataagactattaaaacgtatagcgtttttggatttacaagatgggacttctgtgaacccattaagaatagttattccactcacaaatactgatgaagtacagttcctaaaaattctgaaaactggtcaaactttatctatatctaatgctacctggcaaagcacccctaatagaaaacaaccttttgaattgcaaatcaaaaatcctgtcaagtcaattaaacttgtgggtcccgtttcagaaaactatccattacaaaagaaatatcaaaccttacgttatttaaggtccttacctacactaaaatacagaaccgcttacttaagtgcaattttacggttaagatcctttgtagaattccagttcatgctatatttccagaaaaaccacttcaccaaagtttcaccaccaatattaacttcaaacgattgtgaaggtgccggcgagttgtttcaagtctccaccaatacgtcgccaactgcatcctcgtactttgggaagccgacttatttgactgtgtccactcaattgcacttggaaattttagcgttatcactgtcaaggtgttggacgttatctccttgctttagagccgaaaagagtgatactccaagacacctttcggagttttggatgcttgaagtggaaatgtgctttgttaatagcgtcaacgagctaacatcgtttgttgagactacaataaaacacataattaaagcttgtatagataaccaacaagaactcttgccgaagcaatttatctcttcacaagaaaataatgcatcgtcagagctatcaataaatcaagagacacaacaaattaaaacacgatgggaagatttaataaatgaaaaatggcacaatataacgtataccaatgcaatagaaattctcaagaaacgccacaatgaagtttcacactttaagtatgaacctaaatggggacagcctttgcaaactgaacatgaaaaatttttagccggagagtattttaagtccccagttttcgttaccgactatccacgtctttgtaaaccattctacatgaaacaaaattccactcctgacgatactgttggatgctttgatctactggttcctggaatgggtgaaataattggtgggagtttaagggaagatgactatgacaagttatgtagagaaatgaaagcacgcgggatgaatagatctggagaattggactggtatgtttctctgagaaaagaaggaagtgcaccacacggaggctttggtctagggtttgagagatttatctcatacttatatggcaaccataatataaaggatgccatacccttttatagaacatctgcagaatccatcgatttttga

SEQ ID NO: 96 - DNA, 1395 bp, Saccharomyces cerevisiae
atgccactgaaacgctccttaaaagaatcaatcgaaagattatctagttttcaatcaaaatataatattttcactagtatcaatccatctccttattcaatcacaaataaaaaaggtacaaaagaaacattgacaggttgcgttgccagtatcaaggataatatagttacaaaagattttccaaccacatgtgcctcacatatacttgagaatttcaagtctcccttcgatgcaacagtagtgaaactattaaaacaggcaggagtgcatatcttaggcaagacaaatttagatgaatttggaatgggttctgggggagtacattcaataagagggcccgtaattaaccctttatatcctcatgaggacaagaagatcatgggaggttcatcgtctggagccgctgcaagcgttgcttgcgatctagtcgattttgccttgggaacagatactggtggctccgttaggctccccgcatgctatggatctgttttaggattcaagccgtcctatggacggttgtcaagatttggcgtaatagcgtattctcaatctttagacactgttggcatacttagcaaaaagataaacgtcttgcgaaaagtatttcatacgctcgataaatatgacatgaaagatcccacttccttaagtgtggaattgcgtgaattgatagaagggaataaaaaggttaggagacctttaaaggtaggaattgtaaaggaatttagccatgaaagtatgcccattgggttccacagattatatttatccttactagaaaagctgataaatttagggcttgaaatataccctgtatccattccatctgtaaaaaactgtttaccgatttattatacgctatcacccgctgaagcggcttccaatttatcaaggtatgatggtatcagatatggatacagagattcagaactcgatataaaagacggtattttattcgcacctactaggtccaaattcggaacagaagttaagaatagaatcattctaggtaattataatctttgttcagatgcatttaagaacaattttattaaggcagaaaagctacgagttaatttgatcgatgaatttgacgggatcttcagatttccaaatgttttaactaattctaagggaaatccagacggcctagatctactgatagttcccacatcgtccaagcttcctggatccataagggacttcgaagaagaggaagccaaatctccggctaactcgtatataaacgatgtctttactgtcccaatgtcattggctggactaccaagtttatcaatgcctttaaaagagaagaccccgattggcttacaggtagtaggtcaatatggtgatgattccactgtgttagattttgtcgagtcaatatcataa

SEQ ID NO: 97 - DNA, 1758 bp, Saccharomyces cerevisiae
atgctgggccgggcactaatcaacaagtatggatttttaattcatcctaggagatttgtacatttaaacgataagtcccttgatggaacatttatactaccttcgaagaaaaatcatatgtatgatgttccaactaatgatccaagcggtatcctgaatgcctcggatatcgatagaatcaataatttgccatttttcgataacaccagtccgacaaaagagacaaatactaaggaaggagcattgttgagcgaaaaattggccagcgttaaagaattatttggagaagaccctgaaaatccttcgtttatcaattacagattccccagaggtttagagaatccctattttgacattcaagtaaatcagctaaagaaaaaacgattatccgtcacacaattatgcactacgcagaattggtgcgagttaaggaatttttacgacttttattctcaaaatttgtctaaccaactactgaacttaaaatttcaggtgcagaaaggtaaaaagattcataaatcactagaggacgaaacgcatcctgaattaaatcagtataaaagctttacccacaatttccttgcactgacaaaattatctatggatattgataatgatatggatgccctgctagataactggttcaactctatcaatagattggtctctttattcaccaaaggtgacggtcatgcaagagaaatagtttgtcatggcttcattaacttagaagatggcaaactggtggagcatttgctgaacagtgatagcaagacaaaggaaaatgtgattatatcaggtgttattgaccacttaactcttagaaataggcacaatcatcaagtacagaaaggtgcagctcatcttgatacggaatatcagagctggggtaatatcctcactaatttactatcaaatctaaaagaactgaagtcgaataacgagattgttatctctgatatcaaaactaggtcagtaccaaaaataccatctattgagtccgttatagaatcctctaaattacagacaatgtattataagttttttttttcccaccttagtcaagacatgacacaaacctaccatagctttttaatcaatgcccagagaagaggactcgacgttgatgcgcccataaatcctaccaagattttaacattcatactaacaaacccactcttcgctaatgatgtgaagaacctcctgtacggactaccgataaatcattctgctttcgataacgacgccaaaggctctaatacatttgatatgaccgcgttcaatgatttgctggatagaggtcccacatcttttaacgttccgattgagcaagacgaagatagttcggagtcaacaaaatgtgtatctttgcgagattatggacatttttatacgaaatggaagacacctctaactttgaaatactttgcggcaaggttatcacagatctattttattgtgggtaatctcgtttcgaatgatttaatgatagaatattattaccataacgataatttccacaatatcatcttcccatatgatcccttgaaactgggaacccatgcccatgattctgcaatggtttggtttggtggcagagatatgcatccaattgagcctactcaaaaaaacttcaacacatactgtaagttttgtgactacagacacgtttgttcttggaaaaataaaaatgaattgaaacttattgatctagggaaagaattaaaaaaaattattcttgaatcaagtatgaagtag

SEQ ID NO: 98 - DNA, 894 bp, Saccharomyces cerevisiae
atgtctggaagctatcaacatttatcaaacgttggatcgcgtgttatgaaaagattggggaatcgacccaaaaactttcttcctcatagtgaaaagttcattaaaaaatctacgcctgagttcatgaagtcagatttaaaggaagtagatgagaaaactagtttcaagagtgaaaaagaatggaagtttatacctggcgacagagtagtagtaatgagcggggcttccaaaggaaatatagctgtaattaaatctttcgataaaagaacaaattcattcatattagatgagaatgggcccacaaagactgttccagttcctaaacaattctggcttgaaggccaaacatctcacatgataacaattccagtttcaattttgggaaaggatttaaggctagttgctgatattgacgatgaaaagaccccaggaaagacaagaacggtggcagtaagggatgtttccttcaatggttcctactatgatgcagactacaaaaaggtaatgccatatagatgcgtaaagggtcaaccagacttgatcattccatggcctaaaccagacccaattgatgtgcaaacgaacttggctaccgatcctgtaattgctagagaacaaacattttgggtcgattccgtagtgagaaaccctattccaaaaaaggcaataccaagtattcgcaatcctcattctaagtacaaaaggggtaccttgacagcgaaagatatagctaagctagtagcacctgagatgcccctaacagaggtaagaaaaagccatttggcagaaaaaaaggagttggctgagagagaagtaccaaaactaactgaagaggacatggaagctattggggcgagggtttttgaatttttggaaaaacaaaaaagagaataa

SEQ ID NO: 99 - DNA, 1821 bp, Saccharomyces cerevisiae
atgaatacattaaggtgtttgacacaagcactatccaaaagtgggagagaagctccaaaactatatcagaaagtaatatttccaggcctgttcagagagggtataccaattgcaaatgtcaagaaagttgatgaaaaaataatagatagcccaacatcaacgtcagttaatggagaggcaaaaaaaatagtacggcatggtgtgaagtatgaaagggagcaagttaaggaatacctttcaagcctgccgactttaacgctttctaggaaacaaattagggatgactatgatgaggaacgtgcaaaacgaatgtatatgttctctaaacaaacgaattcctcgaataaattccagaaacttcttacagcaaagtctcaagagtttactagagaactattgacgctactgatagattgcacatcgaatgagaagaattcagggccagaacgcttcactcgtaagtttcttaagtttagtaatgacgagatccctcctctccctgacttttcaaagaaccctcaattgtttgaaaattatattggaatactaagtcatacaaagttcaattttcgttcatcttccaaactaaatggtattgtgcgtaagatgctgcggcacctacttcatccaaccaataagacgactctgccgttgcgttctgctcaagtttataatgactcaatatactttttcagtgagcacttcgactttgcgtcttgtagagaaatatttgcacaaatgaaggctgaggggactaaaccaaatacaatcacttttaatcttttactgcgtaatgtagtgaaaaactcgcatattagaaaaacgaagcaccctgacgatgaggtccttttttatcttagaagtatgcgtaatcatggagtttttgcagatgtaataacttggacgacatgttataactttcttcgtgatgaagtttcgagacaattgtacattgttcaaatgggtgaacatttaggaaattttaacgtaaattttgtttatactgtgctaaggaacggagattatagagcagaggactgtttaaaagttttagcagcaaattcactaccgatttcccgtaagacgttctatctgtgcatagaacgactactaaacgaggaacaacttgaaaccgcatctaaattacttgactatggttttcagcacttgaaaagtaattttaagttggactcggaggcaatgaatcatttcatgcgggtatttgctaataagggaagaagtgatttggcattcctttgctataatacttgtcgtaagatttacaaaattaaacctgattctcagacgttcgaaatgttatttaaagcgttagttaggaatggcaacaccaagaattttggggcagtcctacagtatatcaaagacttaaaggttagtgaaggatttggattgcggacaagttattggagaaccaaagcagactcaattttcaaatttggtagtcctaatacgctatcagaaaagtcaattgaaaaagcaagaaaattgttgggcaacctgattgcttccgagggtgagttctcttggaaaatttggaaagaaagtgattcttcacaaaaaaaaatactacgttttttgggttgtatccccactactttacgctgcaccaatacagcacaagatcatcagaagcccacaaacctaccctcaaacatttcccagaaaaaaagagaatatcgaaatcgggttaaagcaatagccacaaaggctgctttggaaaaaagaatggcgtatatcaaagacaatgatgtagctttcaaaaaagagctagtaaagaggagaatagttggggaggtttga

SEQ ID NO: 100 - DNA, 990 bp, Saccharomyces cerevisiae
atgagtgaagaatttcctacgccgcagcttttggatgaattggaagatcaacagaaagttaccacgccgaatgagaagagagagttaagttctaatcgtgtgttgaaggatatttttgcgggtaccatcggtggtattgcacaagttcttgtagggcaaccgtttgatacaacaaaagttaggcttcaaacagccaccacaagaacaacaacgttggaggtattgcgtaacttggtaaagaatgagggtgtgtttgcattctacaaaggtgcgttaacaccgttattgggtgttggtatatgtgtctcagtacaatttggtgtgaatgaggcgatgaaaagatttttccaaaattacaacgcttctaagaacccgaatatgagctcacaggacgtagatctttcacgttcaaatactctgccactatcccagtactacgtttgtgggttgacaggtggtgtagtcaattcattcttggcatcgcccattgaacaaataagaattcgtttgcagacgcaaacatctaatggtggagacagagaattcaaaggtccttgggactgtattaaaaaattgaaagctcaaggtggattgatgaggggtttatttcccacaatgataagagccggacatgggcttggtacatattttcttgtctacgaagcattggttgccagagaaattggtacgggactaacaagaaacgaaattcctccttggaaattatgcttatttggtgcattttccgggacaatgttgtggttgacagtatacccattggacgttgtgaaatccattatacaaaacgatgatttacgaaaacctaaatacaagaattccatttcttacgtagcaaagaccatttatgcaaaagaaggtatcagagcctttttcaaagggtttgggccaacaatggttaggtccgcgcccgttaatggtgcgacattcctgacgtttgaattagtcatgagatttctgggggaagagtag

SEQ ID NO: 101 - DNA, 1482 bp, Saccharomyces cerevisiae
atgagtaacgatgaaggagaaacatttgccacggaacagactactcaacaagtttttcagaagctaggtagcaacatggaaaatcgagtttgttttgattgtggtaacaagaatcctacttggacttcggtaccttttggtgtgatgctgtgtattcaatgctctgctgtgcatagaaacatgggtgttcatatcacctttgtaaagtcttctacattagataaatggacaattaacaatttaagaaggtttaagttgggtgggaaccataaggctcgtgattttttcttaaagaacaatgggaaacagctactaaatacagcaaatgttgacgcgaaaacaaaatatacgagccctgttgctaagaaatataagatacatctcgacaagaaggttcaaaaggatatggagttatacccatcagaattagtcttgaacggtcaagattcttcggactcaccgttggacaccgattcagacgcttccaggtcgacttccaaagaaaatagtgttgacgattttttctctaattggcaaaaaccaagctccaattccagttctaaactcaatgtaaatacaggctctctcgccccaaaaaacaacaccacaggcagcacacccaagaccactgtcactaaaacgagaagttccatcttaacggctagcagaaagaaaccggtattaaactcgcaagataaaaagaaacattcaattttgtcttcgtcaagaaagcccactagactaaccgcaaagaaagtcgacaaatcacaggcagaagacttgtttgatcaatttaaaaaagaggctcaacaagaaaaggaggacgagttcactaattcgtcttctagcaccaaaattagacaaaatgattacgattcccaatttatgaataacagtaaaggcaacaataataacagtatagatgatattaatacccagcctgatgaattcaatgattttttaaatgacacttcaaacagcttcgacacaactagaaaagaacagcaagatactttaacacccaagtttgccaaattgggatttggtatgaccatgaatgacgccaatgatttggcaaagcaacaaaaagaatctcagaaaatagctcagggaccaagatatacgggcagaatcgcggaaaggtacggcacacaaaaagctatatcatctgatcaattatttggtcgtggctcatttgatgaagcagcaaacagggaagcgcatgacaagttgaaaacatttgacaatgctacatcaatttcctcttcttcatactttggagaagataaagaagtggatgaatttggtaatcccatcaatagtagtggaagtggagcagggaattttgatggccgtaattctaataacggtttcatcgattttaatgcctccgcagatgatgaattgcagatgttgagagacgttgtagaacagggtgctgaaaaactcggtagttatttaagggactacttaagaaaataa

SEQ ID NO: 102 - DNA, 1164 bp, Saccharomyces cerevisiae
atgggtgttagtgcaaatttgttcgtaaagcaaagagggtcgacaacagctctgaagcagccaaaggaaataggattctattcaagaactaaggatgaagaatatttaattagcgatgatacgaatttgaattactactatcttccagacgctgaattggacaggaagttggatctttcatcaggatttcagaaatttaaggattattacaaagattttgaagacaggtgttccctgcgtgggctattagaaaccatagaaagctctgaaagacataaaggaaaaaaaattaatgctgatataattacttttagaggaatagcacggaaattaatttcatgtgcatttgactctccatcttttaatacagttgatcttcgtatcgtttcattcaacggccaactttttatcaaggaagtgcctgaagctgtcaatgcagctaaggcttcctctgcaactgaagcagggcgtaatataaatcaagatctgaatgtatttacagggtacaagtttgaaactttggctactctttccaatccgttgcagtatacaccccgtgaagtgatagaaaaaagaactaaaaggattgtatcgcatggagatgaatacattagtgttgtgagaactggtgtaggtaactgcaagttaattcttggtgctgaggtggattgtatatttgactttaaagaaaacggtagagacaatttaaaacattatgccgaattgaaatgtacgcagcaagttgcgaacatatcggatacgcataagtttgagcgtaaactgttccggacctggctgcaatgcttcttagttgggatcccaaggatcatatatggttttaaagacgaccattatgtattaaaaactgtggaagagttttcaacggaggaagtacctgttttattaaagaacaataatccacaagttggaagtgcatgtttagaagccattaaatggtatggtctccttactgagtggcttttgaaaatgataccaagagatgaggatcctcattctcaaataagagcatttaagttggttttcgaaaataatcacctaagattatccgaaattgaagaaagtgatgaggaatattcaggtttgattgatggagaacatatattatcgaacgggtttaaggagtggagaaaatctttgaaataa

SEQ ID NO: 103 - DNA, 1932 bp, Saccharomyces cerevisiae
atgtggaaattcaacaaaaagctcgcccgtcttacttaccgtctttattcaagctcaggcccaagctcgccccttcatgggaagaagaaactgccgcaaaatctgaaattcgtcgtcttgaaccctacccaatctgggctagtgaaaaatgatcagaagcagcctagacacaggccgtctaagaaacgtagccataaggaaactggagataacaacctcgatttcggctcgaaactccttgtcttcgaaaagcagaattcactagattccgcattgaactccatccgattgaagaaaccgacaagcgccagtctgccctccctggaatacaatgcccttctccaatcgcttacatcaagctacaatcgctaccaactgcgcgagttcatctccacacatcagccagactcctcttcacatctgacgcactggaagaaaagcaagctgtcccaatatataattgaaaaaatctggaattgccaaccaatttcaacacccaccactcccacaggcatcaagtccacatcattgactttccagttcgattctcctagagaaattttccttttactcatcactcaaaatggtaaaatactcaccaacttcaataagctcgggctgacattcatcatctccattcaggataacgaattgaccgtcaagggttcgcctagcttgctcaaatacgcagaaatatccttaaacaaaatatggtcaaacatcactcatgaaaatgtgcgtatgtattccctcatgccatcaaaagacgtcatcaaccttattcagaaggaaactcacactttttttgaatacctcccggacttgcaaatgtacaagatttcagccctaagcaccaagaaaatatccatggccaaagtctttttattaaatgccgtcgcttctaatcctaacacaactcaacaccaccacacaatagcctcaccagcactcaagacagaactctatccgttcaacaatactctagagaacttggactggctaaacaagtcacaagattgggcaagattacagtcagttgtccctaaaaattgcacagatctgatgactcccactgaaaacgccacaccagaattgacagatgcgcaggtcagccaattcgaatcttccttgtctaaaaatataccatctctctccccttccgactctatttcccaatctttatcaataacgctaggtcactccttacagtccgcgtctttttctagtattttccaacctctaatacataagagttttatttcaaaattactgaatctaccaatgtacaaagaatcatcatcatcagccgtacctgtgcctgtgcctcttgatcaacatctaataacaaatgcgcatcaatccttcatacagctgaacttcactcctgtgccgcccacctctggctcctcctcgtccccttttttgcagatttggttcgagattgacgaattcgataacattgttaccacctcaatgaggcctcttttgaaattacaagaaaattctgttatcttaagaactccacaatgccagacagactataagatcacgtcagattacattcaagatcttttacctgatttcgaccagaccaatccggatgcttggttatctgaacaaaaaggcctccaagaatttcttctcaagtcacactggaagttaaacaaatatcagaatcttatgaaaaaaatcaacatttctttaccagataacctaatacagcaataccaattgactgatgttttgacccatcgtgtcttgaatttacgatttcctacgaacactgcccaagatgataaatacatcattcaatattcagatatcagccgcggatttttgaacaatggctcctacagacaattagatttcatcaatgtgaaccccagtgaaacctcactaaaaacctttatcaatgatgttttaagcttttaa

SEQ ID NO: 104 - DNA, 333 bp, Saccharomyces cerevisiae
atgaacgtaccaaaggcgagactcctaaaagtagcagagttatctgcgaagatatttgatcaaaatttcaacccatcaggtataagaaccggttctaagattctaaatgagcgattaaagggcccttctgtggcaagctactacggcaatccggatatcctgaagttcagacatttgaaaacattatatcctgatattgaatttgtagaccttgaggagcaatatcgattatctatggtagaagcaaagaaacgtcgtggtaaaggtgctcctaagaagatgaagaaggatgcagctgcaactgcaaaggggaaggggaagaaaaaaaaatga

SEQ ID NO: 105 - DNA, 2253 bp, Saccharomyces cerevisiae
atgagtataattagttcttggttacttgtctctattatttgcttaactacttctattgtaacaaaattgcaagctgctggagttactacccatctattttatttaacaagaggtgctcccttaagtttaaaggaaaactattatccttggttgaaagctggttctttctttccggatgctttgtactcatgtgcaccttcaaataaggattggtctgattttgcagaattcactcattggcccaatttcttaatgattgcagtatcttattggcagcaaaagtatggtcagaacgatcgacttcggggaactcacggctcactcgcactaaagtcatttttaataggggtattcactcatcagatagttgatgtttcctggcattcgttagttaccgattatagaatgcacggactactccgagttctcagcgaaacagaatttgatggagatattgaaactgctcatacgtttttagacgttatgggtgaattcctcaccttaaataatgtaatccgtgacagtaataataacgaaaattgggattttttaactcgctctgattggaaattgcccagagaagaggatttaatggaaattataaggaacgctggactttcaaaggaaaagttgtcctacgcagaactggaattttgcgttaaaagaggaatggcagcggctattagtgaagggtacctattccgctctcaaagaaaccaattgttaactaatatttactcaacatcccccagggctaatgatctaatactgaatcattggctaggagggcagtccaatttggttgcaatgctccaaagatgcgtgcctttcttcgagactttgtttcatgacgagaatactaatgaagctcaagcagaagaactgagattatgtgctaatttaccgccagtatcacaaaaacggataaatgcacgacctttggtctcttcattaaaagcacgtaagggaaatagccacattgtcgtatctcccatgaaatcattttccgattttggcacaagcctgactatgggaaaatttcgagaagacaacaaggattatctcgctgtaagtgcaccacttgaagacacagttggtgcaatctatatagttccatgggatattctaactgtagcaagaaaagaggattttagcattttacaacccatcactgccatgtacggttcaaaggtcgggacatacaaggcaagtgatgttgactacctactcgtttcacagcctggtacctgtacaatcgacttttacttcaaaggagtaaaaatactgacaatcaaggatgagaccaccgaagaagcacatcagttacaatttgctgtaacaggtaatttttatgatgataagattcctgatttggtggtgtcaagcccaagttatggtgccaacgaaactggaattgcaacatttatcccaggatcttccataataagctatttgaccaattcagacaaatatcaagtagttgatatctcaacttttaagggcgttattaaccttgacgggtaccctatgaaaataccatttcagcatttcggggcaacaattcagatatctgacacgactgacaaacaaaagttaatatatattacatgtcaaagtcttggtactgtttttgtttattcgagtaatgatttacacgatttgtccatcccaatatattatataacgaaaaatggcgtcattccagcaaaggacagtgaccacgttgagtggcatataattccctcaaaagaacatggtatgttcggtgcagccatatattcttggaattttgaaggtatgagttttgtagccgtatctcaaccaatgtttgacactgtgtttatatatattgagaagtctgggcaaatagagtttttcttaaaactggttctaaagattaagacaaaatcagattcaatcccggatgagtttggttcatctttgctgtttaatgacgaagaaaagaaattgtacgtttcctccccaggcagtttcgatgcaagaggaagtatatggaaaattagtatggatgagctattaaaggcgggaaacgatccgaagcgcaaaacccttttgattaataatttaaggcatctcatgctcataaatcctgacaaaagtagcaagggtgtgtcaaattttggtaatagcatgattttgggtccccaaaatcaccttatagtaggcattccccaatatgggtatggaaattttgatcacatgcagctaaccggtagaatcctggtactttaa

SEQ ID NO: 106 - DNA, 645 bp, Saccharomyces cerevisiae
atgtcatttattcagaggaggttattgtcgcaaacactttttctgcgctctcaagtggggtctttaccactttatatatctcctgaagttcaggtttccataaatgcattatccatgcccaggattatcagaaaaggtagaacttcaatgaatatatcacagaatatcacagtaaagggacccaagggtgaattgtccgtggaggtccctgattttttgcacttggacaaggatgaaaagcacggtaaaataaatgttacagttcagaattcagaagataaacatcaacgttcgatgtggggtacagtaagatcacttataaataatcacattataggtgttactgaggggcatcttgctgtccttagatttgtagggacaggttatagagctcaattagaaaatgatggtaaatttgtcaacgttaaggtgggtgcatctatcaaacaaggattggacgttcctgaaggtattgttgtcaaaacacctgctcccacgtctctgattattgaagggtgcaataagcagcaggtccttctttttgctgccaaattgagaaaattccatccaccagagccgtataaaggcaaaggaatatacgttaacgatgaaacaataaagctaaaggacaagaaaatcaaataa

SEQ ID NO: 107 - DNA, 1482 bp, Saccharomyces cerevisiae
atgctaccctatatggaccaagtactaagggcattttatcagagcacccattggagtacgcaaaatagctacgaggatataacggccacatcgagaacattattagatttccgaattccctcagcaatacacctgcaaatttccaacaaatctactcccaatacattcaattctttagatttttctacgaggtccaggataaatggttctctgagttatttatactccgatgcacagcaattggagaaattcatgcgcaactctactgatatcccattacaagatgccaccgaaacatacagacaattgcaaccaaacctcaatttcagtgttagtagtgcgaatacgttgagtagtgacaacaccacagtcgacaatgacaagaaattactacatgactcgaaatttgttaaaaaatccctttattatggtagaatgtactaccccagctctgatttagaagcaatgataataaaacgactaagtccacaaacccaatttatgcttaagggtgtcagtagtttcaaagaaagcttaaacgttttaacgtgctattttcaaagagattctcaccgcaatttacaggagtggatattttccaccagtgatctattatgtggttatagagtattacacaatttccttaccacgccttccaagtttaacacctcactgtacaataattcttcgttgtcgcttggtgctgaattttggttagggttagtaagtttaagccccggttgttcgacaactttaagatattacacacattctacaaacacaggacgaccactaactttgacattatcttggcaaccattattcggccatatatcctccacatattcggccaagacagggacaaattctactttttgcgcgaagtatgattttaatctttattcgattgaatcaaatctttcatttgggtgcgaattttggcaaaaaaagcatcatttgcttgaaaccaataaaaacaataatgataaattagaaccaatctccgacgaattggttgatataaatccaaacagcagagcgactaaactactgcacgaaaatgtaccggatctgaattcagctgttaacgatattccttctacactagatatacctgttcacaaacaaaagctattaaatgatttaacttatgcattctcgtcgtcattaagaaaaatcgatgaagaaagatctaccatcgaaaaatttgataacaaaataaatagttccatttttaccagtgtttggaaattaagcacgtcattacgtgacaagactttaaaactattatgggaaggcaaatggaggggatttttaatatctgccgggacagagctggtattcactagaggctttcaagaaagtttatccgatgatgaaaagaatgataatgcaatatctatatcagcaactgatacagaaaacggcaatataccagttttcccggcaaagtttggcatacaattccagtactccacatga

SEQ ID NO: 108 - DNA, 1149 bp, Saccharomyces cerevisiae
atgagaaaagaattaaaataccttatctgcttcaacatactcctcctgttatctataatatactacactttcgatttgctaacgttgtgtattgacgatactgttaaagatgctatacttgaggaagacttaaatccagatgcacctccaaagcctcaactaatacctaaaatcatacatcagacttataaaacggaagacatccctgagcactggaaagagggtagacaaaaatgtctcgatctacatccagattacaagtacatcctatggacggacgagatggcctatgagtttataaaggaagaatacccgtggtttctcgatacttttgagaactacaaataccccatagaacgtgccgatgccattcgttactttatcctgtcccattatggtggtgtatacatcgatttagatgacggctgcgaaaggaaactagatcctttgttagctttcccggccttcttaagaaagacttcacctttaggtgtctcaaacgatgtcatgggttctgtgcctagacatcccttctttttaaaggctttaaagtctttgaaacactatgacaagtactggtttatcccttacatgactattatggggtctactggtccgttgtttttaagtgttatttggaagcagtacaaaagatggcgcatacctaagaacgggacagtaagaatcctacaacctgcttactacaagatgcatagttattcatttttctccattactaaaggctcttcatggcatttagatgatgcaaaattgatgaaagcactagaaaaccacatcttatcctgcgtcgttaccggatttatttttggcttctttatcctatatggtgaattcacgttctattgctggttgtgttccaagaatttcagtaatctaaccaaaaattggaaacttaatgctataaaagtaagatttgtcacaattctaaattcattaggcttaagactaaagttgagtaaaagtaccagtgatactgccagtgccactttgctggcaaggcagcagaaacgattgaggaaagattccaatacaaacatagtgttactaaaatcttcaaggaaaagtgatgtttacgatttagaaaagaatgattcttctaagtactcactgggaaataacagctcgtaa

SEQ ID NO: 109 - DNA, 2031 bp, Saccharomyces cerevisiae
atgctcagaagacacggattattctggttaaagacatgtccccgtctaaatgttcttctaaatcaatccataccgatacctcatttgcttcatagccgtgatatttgccagcaaagatggtatgcaaaagggaaaagaaggaaccagatttccaagaaagagttaaagcctctcaatttttctattccgaattatatttctgtgaataaattggccaatttgttaaattgtcgtgtggaaagattaatcaaagatttgacagcattaggtttcgaaaatattacaaccacgtatatattatcaaaagaatacgtcgaattgattttacaagaatacaactttgcccttccgaatctgtcaacgtctacaaatttagataatgtttatgatgagctaaaatctcctgtaaacccaaaattattaaccaaaagagcccccgtggtaactataatgggccatgttgatcatggtaagaccactatcatagactatctaaggaaatcttctgttgtggctcaagaacatggtggaatcacgcaacacattggtgcctttcaaataacagctccgaagtctgggaaaaagatcacatttttagacacccctggccatgctgcctttttgaaaatgagagaaagaggtgcaaatattactgatattattgttttggtggtatctgtagaagattctctgatgccccagacattagaagccataaaacatgccaaaaattcaggaaatgaaatgataattgcaataacgaaaatagatagaattccacagccaaaagaacgtgaaaaaaaaattgaaaaagttataaatgatttgattgttcaaggaataccagtagagaaaattggcggagacgttcaagtgatcccgataagtgccaaaactggtgagaacatggaccttttagaggaatcaatagtcctattaagtgaagtaatggatataagagctgaaaactcccctaaaactatcgcagagggttggattatcgaaagtcaagttaaaaaacaggtcggaaatgtggcgaccgtcttggttaagaagggcactttacaaaaagggaaaattctaatttgtggtaatacattctgcaagatcaaaaatttaattgatgataagggcataccgattcttaaggcaacaccatcatatgctacagaggttcttggttggaaagatgtaccccatgttggagatgaggtcattcaggtaaagagcgaagcgatcgctaaaaagttcatcagtaaaaggcaagatcttatcgaagttcaaaaaaactctagtatcgttgaaaaattaaatgaagaaagggcccttgccaaagagcaacacctaaataaggaactggaacatgagaatactgtccaggaacacgaacaaaatactggtcctaaacttattaattacatcatcaaatgtgatgtctctggctcagcagaagcggtttcagaaagcatttcctctctaggaaatgacgaagtgagatgtaatgttatctcttcttctgttggtatacctacggaaagtgacttaaagatggcgcaaataacggagagtacaattctatgcttcaatttggggaatttgccgagtgaggtgatcaacaatcgtgctggcatcaaaataaaacagtacaatgttatctataagctaattgaagacgtcactgaaacactaactgaaaatctaaagcccatctttgaaaaaaagatcgtttcaactgttgacgttcgtgaaacttttgactttaggttaaagaaaaaaattattaggattgcaggttgcaaagtaaacaacggtgtcatcaaaaagaattcattggtgcaggtagttagaggtcctaacgaagatgtaatttttgatgggaaaatctctactttgaagcacaataaagatgacgtagcagaagtttcaaaagggcatgaatgtggaatcacttttgagagtggattcgagggtttcaaaccaggtgataagatcctagtttatgaaaacgtgcgagttcctcgttacttgtaa

SEQ ID NO: 110 - DNA, 198 bp, Saccharomyces cerevisiae
atggccgtacagacaccaagacaaagactggctaatgccaagtttaacaagaataacgaaaagtatagaaaatacggtaagaagaaggagggcaaaactgagaaaaccgcacctgtgatatccaaaacttggttgggtattcttctgtttcttctcgtaggtggtggtgttttgcaactaatcagctatatcctatga

SEQ ID NO: 111 - DNA, 1671 bp, Saccharomyces cerevisiae
atgttgaatctgtgtcatgctcttcgaggcgtacgtcagttttcctgttctgtgattgtgaaagtgaaatgtgcgtcatgttccataaaactacaggatcaggatcctagtaaaccaggatattatacgaagccaaaaagtctacccgactcaaaactaaatcctgacctccaagatctgaagtatttgctattcagtcaagacatacagctctcgaaacaggcaactcaaaatgatcctgacctcaaaaccaaaagagatttactattgagagtgatttgcaaaaggtgcagtaatgcccttcatcataacaattataacccggaagagtttcccgaaagcacattaaatgatattctgaactacgtccccagaggctcaaatgttatgcatattgtcccctttgtagaatttcccttacatttagaccccaatgttttgaaacgaaatgatttagatactacattagtgttaacgaaaagcgatcaagtttttaaagataaaaatgctgtcagcaaaaaggttcctatattcatgaagcaatttttaaagaacactttaagaattgactcaaataaaacattcgccatatctgcgctgaaaaactggaacatatccatgttttacaactactttaaaaactacacatatttgctaggcaaccctaatgttggtaaatcaactttgatcaatactctactacagaaatatcttggatataaggttaaaattgattctactgggaaaattaactccccctctgaagaagtgatgcaagaagcttttacgaacccgaagaattttttcaagattcaagctgctggtgtatcacatattcccaatttaactagatccgtacaagcctaccaagttggaggtaaaatactttttgatcttccaggctattcaacttccacctccagattacgcctagaagaactaatcgacgagcgttggcttcaaaggctgcgaaaaaccgatttattcaatcggaagcatataaaacagaagacttatgagtctatgaagggcacatcacagggaggctgttataccgtgggagggattttctacctggtacctccgaaaggatccataaatcagattgtgaaatacataccaggaccatcaaaaacattcaagaacattgaaaaaggtattgatgtattcaactcgtgcaattcatcatctggcacacatccgttatcacggtattgtggaattaaaagcgttatatgcgaaaaaagccagtacaaaaggtacgcaatacctccatttattggaagtatagagatcgttctgaaagatataggatatattttattaaggacaactgggaggtatgagttcaaaggactacatgagatatggatacctaggggtatccaagtaggtataagggaaccgctagaaaacttgattgagtctggctaccagcgttacattgaaacaaatggtaaagagtcaagctgtcccagagatagaccgatcatcagttccctgtatgagatggccccggatgaagctgatactttgaacgcagtgaagaagtcgtatttagaaaaaaccgagaaagatttatctgctagaagatttgtcgacgatgatccttatgatttggttcaacacctagaaaaaaaaaagaatccctattggtactaccaatggtaa

SEQ ID NO: 112 - DNA, 180 bp, Saccharomyces cerevisiae
atgactattgctccaattactggtacgatcaagagaagagtcatcatggacatcgtcctcgggttctccctcgggggtgtcatggcctcttactggtggtggggattccacatggataagattaacaagagagagaagttctacgcagagctagctgagaggaaaaagcaagagaactga

SEQ ID NO: 113 - DNA, 372 bp, Saccharomyces cerevisiae
atgataatgagatacgagaaccagaagaaacacaagaatcactccttgtgctcctcatcgtcatcggcaatggcagaagagtcctcttttgactcttccttgccgtttttcttcttatttttggggaatttaggcaagtttttcttcttatggccgttaaaggatcttgatctaccgctgaagtttttggacttcgaggcctctctctgtaaatcaaactgttttttcgtcaaaacactcagtttcttaccttcatatgacaaaatttcgttggactcatcgtcattggaatacgatttcaaaaaagcttcacattctggaattgtcttaaattccaccaagaccgtaccattaaatttcttgtttctgtga

SEQ ID NO: 114 - DNA, 156 bp, Saccharomyces cerevisiae
atggctgctcaaaagtctttcagaatcaagcaaaaaatggctaaggctaagaagcaaaacagaccattgccacaatggatcagattgagaaccaacaacactatccgttacaacgctaagagaagaaactggagaagaaccaagatgaacatctaa

SEQ ID NO: 115 - DNA, 408 bp, Saccharomyces cerevisiae
atgtttttacgtactcaagctcgtttcttccatgctactaccaagaagatggacttctcgaaaatgtccatcgtcggccgcattggctctgaattcactgaacatacttctgctaataacaatcgttatttgaaatatagtatcgcttcgcaaccaagaagagatggccaaaccaattggtataatatcaccgttttcaatgaacctcaaatcaattttttgacagaatatgttagaaaaggcgctttggtatatgttgaagcagatgctgctaactatgtcttcgagagagacgacggttctaagggtactactttgagcttagttcaaaaggacattaatttattgaagaatgggaagaaattagaagatgctgagggccaagaaaatgctgcttcttcagaataa

SEQ ID NO: 116 - DNA, 810 bp, Saccharomyces cerevisiae
atgtcgaagttcctacagggatcaatatttagcatatcaaaacttcatgttagatattcttctactcggccttttctagtggctcctagtattgctaacagtataacaaccgaggctccagcgattaatcactcacctgaactggcaaatgcgagaaaatggctaccaaaaagatgcggtttaattactcgtaagaagggaatgatgccttattttgataaatctactggtgaaagaagcgccgctactatattagaagttaataatgttgaagtaataatgcatagaacttcggaggtcaatgggtacttcgcgtgtcaagtagggtacggatcaaggcatttgtctaaagttagcagacaaatgcttggtcatttcgcttctaaagttgttaacccaaaagagcatgtggccgaattccgagttaaagatgaaaagggattaataccacctggcacattattgaaaccctcttttttcaaagaaggccaatatgttgacgtcagatcagtcagcaaaggcaaaggttttactggtgtgatgaaaaggtatggattcaagggtttgagagcatctcatggtacatctattatgcatagacacggtggttcttatggtcaaaaccaagacccaggtagggtactgccaggaagaaagatgcctggtcatatgggaaacgagcatgttactatccaaaatgttaaagttttgaaagtcgacgatgaaaataatgttatttgggttaaaggctctgtggctgggcccaaaaacagtttcgttaaaattcaagatgcaatcaaaaaaacttga

SEQ ID NO: 117 - DNA, 873 bp, Saccharomyces cerevisiae
atggaaaagctattacagtggtctattgcgaattctcaaggggacaaagaagctatggctagggccggccaacctgatcctaaattgctacagcagttattcggtggtggtggtcctgacgatccaaccttaatgaaagaatccatggctgttattatgaatccggaggttgacttagaaacaaaactcgttgcatttgacaactttgaaatgttgattgagaacttagataatgctaataatatcgaaaatttaaaactgtgggagccattgttggatgttcttgttcagacgaaggatgaagaactacgtgctgctgctttatccattattggaacggctgtgcaaaacaacttggattcgcaaaataatttcatgaaatacgacaatggtctgcgaagccttatcgaaatagctagtgacaagacaaagccactcgacgtgagaacaaaagctttttacgcactatctaatctaataagaaaccacaaagatatctcagaaaagtttttcaaattaaatgggctcgactgcatagcacctgtattaagtgataacaccgccaaaccaaaactgaaaatgagagccattgccttattgaccgcatatttgtcatctgttaagattgatgaaaatataatcagtgtgctgagaaaggatggagtaattgaaagtacgattgagtgcttgtctgacgagagtaacttgaacatcatagatagagttctgtcttttctctctcacctgatatcttccggaataaaatttaatgaacaggaattgcacaaattgaacgaaggttacaaacatatcgagcctctaaaggacagacttaatgaagacgattatttagccgtaaagtatgtattatga

SEQ ID NO: 118 - DNA, 990 bp, Saccharomyces cerevisiae
atgtgcagtaggatactcttgtccggtttagtcggactgggtgctggtactggcttaacctatcttcttttgaacaaacattctccaacgcaaatcattgagacaccttatccacctacccagaaacctaatagtaatattcaatctcactctttcaacgtcgatccttccgggttcttcaagtatggttttcctggtccgattcatgatctgcagaaccgcgaagagtttatctcatgttacaacagacaaactcaaaatccttattgggtcctcgaacatataacgccagaatcattggctgcaaggaatgctgacagaaaaaattcctttttcaaggaagatgaagtaattccagaaaagtttagaggtaaactaagagactattttaggtcgggctatgatcgaggccatcaagccccagctgcagacgcaaaattttctcaacaggccatggatgatacattctacttatccaatatgtgtcctcaagtaggagaaggttttaatagagactattgggcgcatttggagtacttttgtaggggattgactaagaaatataagagtgtaagaatcgtgactggtccattgtatctacccaaaaaggatcccatagataataaatttagggttaattatgaagttattggcaatccacccagtattgctgttccaacgcacttttttaaattgattgttgcagaagcaccaacagccaatcctgctagagaggatattgctgtcgcggcatttgtattgccaaacgaaccgatatcaaatgagacgaaattgactgactttgaagttcctatagatgctttagagagaagtactgggctagaacttctgcaaaaagtaccaccttcaaagaagaaggcattatgcaaagaggtaaattgtcaaattgtagtgagagatttctctaacgcggcgatcaaacaatcgaaagatgtgaaattgttacctcctccaaaaaaaaggaattga

SEQ ID NO: 119 - DNA, 315 bp, Saccharomyces cerevisiae
atgctttttgactcatccttccgcgaaaggagaactttttacaatgccgccggctctgttagcgtacccttctacaagcagatagcagaacaaacacatgatatattcaaaaggtgcaatgtcagagaacatatattgcgcccctgtcctgtagacatcagtcatttttcgcgggacttgaatggcgcaccattatcctatacagaaaacattaaatttactgcaaaattttgggcaaacgcttggatagactatgaatccgtcatgaagacactacttgtaaaattatatatatccttttttttcatgtgctag

SEQ ID NO: 120 - DNA, 3564 bp, Saccharomyces cerevisiae
atgaaaacaaagtttgatcgcctcggtacaggaaaaagaagtagaccctctccaaataatattgactttaacgaccagtctgctacatttaaaagaaataagaaaaatagccgccaaccaagttttaaagtaggtttgtcttataacagtttactggatgattgtgatgatgaaaatgaaacagaggaaatatttgagggccgtggtctgcaattcttcgacaaagacgataatttttcaataacagcagatgatacacaagtaactagtaaattatttgatcacgatttagagcagacacctgatgaagaggcgaagaaacctaagaaggtcacgataaggaaatcagcgaagaaatgtttgagcacgaccatattaccagattcattcagaggggtttttaaatttacagaatttaataaaatgcaatcagaggcttttcctagtatttatgagagtaacgagaactgcataatttcttcaccaaccggatcaggcaagactgtattatttgaattggcaatattacgtcttataaaggaaacaaatagtgataccaataacaccaaaattatatacatcgcgccaaccaaatctctatgttacgagatgtacaagaactggtttccttcctttgtgaatctttctgttggtatgcttactagtgatacctcttttctggaaactgaaaaggctaaaaaatgtaacataataattacgacaccagaaaaatgggatttgttaacaagaagatggtccgattacagccgattattcgaattggtcaaacttgtcctagttgatgagattcataccataaaggagaaaagaggagcatctttggaagtaattctgacaaggatgaatacaatgtgtcaaaacattcggtttgttgctttaagtgcaacagttccaaatatagaagacctagcattgtggctcaaaactaacaacgagcttcctgcaaatattctttcttttgacgaatcgtacaggcaagttcagttaacgaagtttgtttatgggtactccttcaattgcaaaaatgacttccaaaaagatgctatatataattccaaattgattgaaataattgaaaagcatgccgataatcgtcccgtactaatattttgtccgactagggcttcaactatatcaacagcaaaatttttattaaataatcatattttttcaaaaagtaagaaaagatgtaatcataatccctccgataaaatattaaatgaatgtatgcaacaaggtatcgctttccatcatgctggaatttctttagaagaccgtactgccgttgagaaagaatttcttgcaggttcaattaatatattgtgttcaacttcaacgttagctgttggtgtgaaccttcccgcatatttggtcattatcaaaggcaccaaaagttggaactcttctgaaatccaagagtattcagacctagatgtactgcaaatgattgggcgggcaggaagacctcaatttgaaactcacgggtgcgcagtaatcatgacagactctaaaatgaagcaaacatatgaaaacttaattcatggaactgacgttttggaaagttcattacatttgaatttgatcgagcatttagcagcagaaacctctttggaaactgtatattcaattgaaactgcagtaaattggctacgaaacacattcttttacgttagatttgggaagaatccagctgcatatcaagaggtgaacagatacgttagttttcattccgttgaagactctcagataaatcaattctgtcagtatttgcttgacaccttagtaaaagtgaaaataatcgatatcagcaatggggagtacaaatctacagcgtacggaaatgcaatgacgcgacattatatatcctttgagtcgatgaaacaattcattaacgcaaaaaaatttctatcattacaagggatactaaatttactggccacttctgaagagttttcagttatgagggtgaggcacaatgaaaaaaaattgttcaaagaaatcaatttatcgccacttttgaaatatccatttttaaccgagaagaaacagagccaaataattgacagggttagtcaaaaggtttctcttttgattcaatatgagttaggtgggttagaatttccatcatacgaaggcgcttctaaactacatcaaactcttgttcaggataaattcctcgtttttagacattgttttagattactaaaatgcatggtagacacatttattgaaaaaagtgatggaacatcactgaaaaatactttgttcttactgcgaagcctgaatggtcactgctgggaaaacacgccaatggttcttaggcaattgaaaacaattggactagtttcagtaagaaggctaatacgacatggaattaccaatctagaagaaatggggcatttgtcagatactcagattgaatactatcttaacctcaagattggaaacggcataaaaattaaaaacgacatttctttgttgccttgtctgaatattagaacaaaactagaaaattgcaaaatagaaaatgaagagttgtggttgacatttaaagttgagattagtgcaacattcaagtctagcatttggcatggtcagcacctctcattagatatcgaaaccgagaaaagctcaggagagttaatcgactttagaagattgcaagttaacaaactacagtcaccaaggggctttagaatttcagcaaaaatatcaccaaaactagaaaaaatagaattctcaattcattgccaagaaatcgccggactcggcaaaacaatcgtatattccactgatcatttggcatcacagttctctgcgaagacaccaaatataaggaaagatctaaatagtttagaaaaatgcctcttctatgaaagcagtagtgacggagaagtggggaaaacgtctagggtttcccataaagatggattagaagaatcgctatcctcagatgatagtatactagattatttgaatgaaaggaagaaatcatcaaaggcggttgaatctgcagcagttattcacccggaggcacactcttcttcacattttagtaatggccgccaagtaagatctaatggcaattacgagtgttttcatagctgcaaagacaaaacgcaatgcagacatttatgttgtaaagagggtattccagtaaagtatataaaagaaaaaggaccctcctcaatcaagccagtatcaaaacctgatcaaatacgccagccactgcttgcaaaaaatatcaacacaacccctcacctggaaaaaagactgaatagtaaaccaaagcagtggcaagaagaaaatacagatattgcgacagttcatacacttccttcaaaaatttataatctatcgcaacagatgtcgtctatggaagcgggtgaacaagtcctaaaatcaggccccgaaaactgccctgaaataattcctattgatcttgaatcatccgatagttattctagtaatacagcggcgagctcaataagtgatccgaatggtgatctggatttcttgggttccgatatagagtttgaatga

SEQ ID NO: 121 - DNA, 3825 bp, Saccharomyces cerevisiae
atggagcaaaatggccttgaccacgacagcagatctagcatcgatacgactattaatgacactcaaaagactttcctagaatttagatcgtatacccaattaagtgaaaaactggcatctagttcttcatatacggcacctcccctgaacgaagatggtcctaaaggggtagcttctgcagtgtcacaaggctccgaatccgtagtctcatggacaactttaacacacgtatattccatcctgggtgcttatggagggcccacgtgcttgtatccgacagccacgtattttttgatgggcacttctaaaggatgcgtactcatttttaattataatgaacatttgcagacaatcctagtgccgaccttatctgaggacccttctattcactcaataagaagtccagtgaaatcaattgtcatatgttccgatggtactcatgtagctgcctcatacgagaccggaaatatatgcatttggaacttgaacgtagggtatagagtgaaacccacttctgaaccaacaaatggtatgaccccaacgcctgccttaccggcagtcttacacatcgatgaccatgtgaacaaggaaatcacagggttagacttttttggtgctcggcatacagccctgattgttagtgataggacaggtaaagtatcactctataacggttacagaagaggcttttggcagttggtgtataattcaaaaaaaattttagatgtgaactcttccaaggagaaattaataaggtcaaagttgtctccactaatatcacgggagaaaatttccactaatttgttgagtgtactcacaactacacattttgcccttattttattatcgccacacgtttctttgatgtttcaagaaactgttgaaccctcagtacaaaattctctagtcgtgaatagctctatttcatggactcaaaactgttccagggttgcttattccgtaaataataaaatttctgttatttccatatcttcatcagacttcaatgttcagtccgctagccattctcctgaatttgcagaatctatattatccattcaatggattgaccagctcctacttggtgttttaaccatatcgcaccaatttttggtattgcacccccaacatgacttcaagatcctgttaagattggattttctgattcacgatttgatgatcccacctaataaatattttgtaataagtagaagaagtttctacctgttaacaaactactcatttaaaattggcaaatttgtgtcttggtcagatattactttaagacatattttgaaaggcgactacttgggtgcattggagttcatagaatcacttttgcaaccttactgtccactggcaaacttgttgaagctagataataatacggaagagaggactaagcaacttatggaaccattttacaatctgtccttggctgccctaaggtttcttataaaaaaagataatgccgactacaatagggtttaccaattattaatggtagttgttcgtgttttgcagcaatcttccaaaaaactagactcaattccttctctagacgtctttttggaacagggtctggagttctttgaattgaaggacaacgcggtatattttgaagttgtagcaaatattgttgcccaaggatcagttacgtcaatttccccagttcttttcaggtccataattgattactatgctaaggaggagaatttaaaagtaattgaagacttaatcatcatgttaaatcctactacgcttgatgttgatcttgccgtcaaactatgccaaaagtataatttgttcgatttattaatatatatttggaacaagatctttgatgattatcaaaccccagtggtggacttgatatacaggatttctaaccaaagtgaaaaatgtgtgatcttcaatggtcctcaagtacctcctgaaacgactatatttgattacgtaacgtatatccttactggcaggcaatatccacaaaacttgtctatatcaccaagtgataaatgctccaaaatacaaagggaactttcagcatttatttttagtggcttctccataaaatggccgtcgaacagcaatcataaactttacatatgcgaaaatccagaagaagagccagcatttccttactttcaccttttattgaaatcgaatccgagtaggttcttagcaatgctcaatgaagtgtttgaagcgtccttgtttaacgatgacaatgacatggttgcatcagttggagaagcagaattggtaagtaggcaatatgttattgatctactattggatgctatgaaagatacgggaaattcagacaacatcagggtacttgttgcaattttcattgcaactagtatatcaaaatatcctcaatttattaaagtgtctaaccaagccctcgactgcgttgttaataccatatgctcctctagggttcaaggtatatatgaaatttctcaaatagctctggagtcgcttttaccctattatcattcaagaacaacagaaaattttatactggaactaaaagaaaaaaatttcaataaagttcttttccatatctataaaagtgaaaataagtacgccagtgcgctttcacttattttagaaactaaggacatcgaaaaagaatataacacggacattgtatccataaccgactacatactcaaaaaatgcccacctggaagtttagaatgtggcaaagttactgaagttatcgagacgaactttgatcttcttctctcaaggatcggtatcgaaaaatgcgtcacaattttttctgactttgactataatcttcatcaagaaatcctggaagtaaagaatgaggagactcagcaaaagtatttggataagcttttttctacgccaaatatcaacaataaggtcgataagcgtttaagaaatttacacatcgaattgaactgtaaatacaagagcaaaagggaaatgattctttggcttaatggtacagttctcagcaacgctgagagcttacaaattctggacttattgaatcaagactctaattttgaagctgcagctataattcacgaacgcttggaaagttttaacctagcagtcagggatttattaagttttattgaacaatgtctaaatgaagggaaaacaaatatatctactttattggaatctttgaggagggcctttgatgattgtaattctgctggtaccgagaaaaaatcgtgttggatattattgattacattcctgatcactctatatgggaaatatccttcacacgatgaaaggaaagatttatgtaataaactacttcaagaagcatttttgggattggttaggtccaagagttcctctcagaaggattcaggtggggaattctgggaaataatgtcttctgttcttgagcaccaagacgttattttaatgaaagttcaggatttaaagcaactgctactgaatgtttttaatacttataaattggaaagatctctttctgagttgattcaaaagattatagaggattcttcgcaagatcttgttcaacagtatagaaaatttctgagtgaagggtggtctatacacaccgacgactgcgaaatctgcgggaaaaaaatatggggagctggtctggacccattactttttctagcttgggaaaatgtacagcgccaccaagatatgattagtgtagatctcaaaactccccttgtcatattcaaatgtcaccatggctttcaccagacttgcctcgaaaacttggcccagaaacccgatgaatattcttgtttaatttgccagacggaatctaacccaaaaatagtataa

SEQ ID NO: 122 - DNA, 1263 bp, Saccharomyces cerevisiae
atgagtcagcaaacaccacaggaaagtgaacagaccacagcgaaagaacaggaccttgatcaagagagcgtgttgagcaacattgacttcaatacggatttgaatcacaatttgaatttatcggaatactgtatatccagtgacgcaggaacagagaagatggatagcgacgaggagaagtcgttggccaatctgccggagttgaaatacgctcccaagctatccagcctggtgaagcaagagacgctcaccgagagcttgaaaagaccacacgaagatgagaaagaggcgatagatgaggccaagaagatgaaagtgccgggagagaacgaggacgaaagcaaggaagaggaaaagagtcaagaactggaagaggcaattgacagcaaggagaagagcaccgacgccagggacgagcaaggggacgaaggtgataatgaggaggaaaacaacgaggaggataatgaaaacgaaaacgagcatacagcaccgcctgcgctggtgatgccctcccccatcgaaatggaggaacagaggatgactgcgctgaaggaaatcaccgacatcgagtacaagttcgcgcaattgcgccaaaaactatatgacaatcaattggtgcggttgcaaacggagctgcagatgtgtctggaagggtcacacccggaattgcaggtctactactcgaagattgccgcgatccgtgactacaagctacaccgagcgtaccagcgacagaagtacgagctttcatgcatcaacacagaaacaatcgctaccaggacattcattcaccaggacttccacaagaaggtcaccgacctgcgagccaggctgctgaacagaaccacgcagacctggtacgatatcaacaaggagcgccgcgatatggatatagtcatcccagatgtcaattaccacgtccccatcaaacttgataacaagacgctgagctgtatcacgggctacgccagcgcagcacagctgtgctatcccggcgagcccgtggcagaggacctcgcttgcgaaagcatcgagtaccgctacagagccaacccggtggacaaactcgaagtcattgtggaccgaatgaggctcaataacgagattagcgacctcgaaggcctgcgcaaatatttccactccttcccgggtgctcctgagttgaacccgcttagagactccgaaatcaacgacgacttccaccagtgggcccagtgtgaccgccacactggaccccataccacttctttttgttattcttaa

SEQ ID NO: 123 - DNA, 2514 bp, Saccharomyces cerevisiae
atgaacgacccttctttactaggctaccctaatgtgggcccgcagcagcaacagcaacagcagcaacagcagcatgccgggctactaggaaagggtacaccaaacgctctacagcagcagctacacatgaaccagctcaccgggatacctccaccgggactcatgaacaacagcgatgtacacacttccagcaacaacaactcgcgccagttgctcgaccaattggctaatgggaacgcgaacatgctcaatatgaacatggataacaacaataacaacaataacaacaacaacaacaacaacaacaacggcggtggaagcggggtgatgatgaatgcctccacagccgcagtaaattctatagggatggttcctaccgttggtacgcctgtgaacatcaatgtgaacgccagtaaccctttattacacccgcacttggatgacccttctttactaaacaatccgatctggaagcttcaattgcatctggctgcagtgtccgcacaatctctggggcaacccaacatttatgctagacaaaacgctatgaagaagtatttggctacacaacaggctcagcaagctcagcaacaagcgcagcaacaggcacagcagcaggtcccgggcccatttggccccggacctcaggctgcaccaccagctttgcagcccaccgatttccagcaatctcacattgcagaagcctccaaatcactggtagactgcacaaagcaagccttgatggaaatggccgacactctcaccgacagcaagacagcaaagaaacaacaacctacgggagatagcactccctcaggcacggcaactaacagtgcagtttctacaccattgactcccaagatagagctgtttgctaatggcaaggacgaagccaaccaggcgctcttacaacacaagaaactgtctcagtacagcatcgacgaagatgacgacattgaaaacagaatggtcatgcccaaggactcgaaatacgacgaccaattatggcacgcgctagatttgtccaacttgcaaatcttcaatatcagcgccaacatcttcaagtacgattttctaacgagactatatttgaatggcaatagcctcacggaactgccagcggagatcaagaacctaagcaacctacgcgttttggacctgtcgcataataggttaacatctctaccagcggaactaggctcatgtttccaattgaaatacttctacttttttgataacatggtcaccacattaccatgggagtttgggaacctgtgtaaccttcagtttcttggtgtagaaggaaaccccttagaaaaacagtttttaaagatccttacagaaaaatctgtcacgggattgattttctacctaagggacaacagaccagagattcccttaccgcatgaacgtaggttcatcgaaatcaataccgatggggaaccacagagggagtacgattctttgcagcaatccactgagcatctggccaccgatttagccaagaggacgttcaccgtcttatcctacaacaccttatgtcaacactatgccaccccaaaaatgtaccgttacacaccgtcgtgggcgttaagttgggattacaggcgcaataaattaaaggagcagattctctcgtacgacagtgatctgttgtgtttacaagaagtggagtctaagacttttgaagagtattgggtgcccctattggacaagcacggttatacaggcatcttccatgcaaaggcaagagccaagaccatgcattccaaggactccaagaaagtggacgggtgttgcatttttttcaaaagggaccaattcaagttgatcaccaaagacgccatggatttcagcggtgcttggatgaaacacaagaagttccaaagaactgaagattatttaaaccgtgcaatgaacaaagacaacgttgcactgttcttaaagctacaacacattcctagtggcgacaccatatgggcggtcaccacgcatttgcactgggatccaaaatttaatgatgtcaagacattccaagtaggtgtcctgttagatcatctggaaacgctgctaaaggaggagacatcgcacaattttagacaggacattaagaaatttcctgtgctcatttgtggtgacttcaattcatacatcaactccgccgtatacgaattgataaatacaggccgtgtccaaatacatcaagagggaaatggtagagatttcggttacatgtcggagaaaaatttctcacataacttggctctcaaatcaagttataattgtatcggagaactaccattcaccaatttcacaccgtcattcacagatgttatcgactatatatggttttctacacatgctctaagggtgcgtgggctattgggtgaagtggaccctgaatacgtgagtaagtttatcgggttccccaacgacaaattccccagtgaccatataccattgttagcaagatttgaatttatgaagacaaacacaggcagtaagaaagtataa

SEQ ID NO: 124 - DNA, 1869 bp, Saccharomyces cerevisiae
atggtacgtcgatggattcctagtggcaggcatcttcgcaataatgacaacactggtgatgatgacgacagcgagttcacaaactcgatggattctgggatgtccataccatcactcagggactccatgaccacgaggtcatctcataacgatcccatcaagcctgctctgatgaacgattccaacaaagtcaaaaatttggagaaggagttgactaatgccaaaatcaagattcaagtactctatgaatacattcgcagaatccctaataaagacggcaatgcaccatcgctgggcaatgacactgattttagaaattcgattatcgagggtctaaatcttgaaataaacaaattgaaacaggatttaaaggcgaaggaagtcgagtaccaagatacgctacaattcgttcaagagaatttagaaaattcggaaagtatcgtgaatacgatcaatcacctattgtcctttatattgacgcattttaatgagcaagatgaaaatgcccatcttcttgataaagaagagagggagaccctggaggaaactttagagctgagctcggattatgttcttgaaaaaatggatactttgtccaagttcatcatccaattcttgcaggactttttgcattccaaaagtcgagcggaatcaaagcaggataaggaagaatttctttcactggcccagtcctcaccagcaggatcacagttagaaagtagggactcaccatcaagtaaagaggagaatactgatggtggataccagaatgacgaaattcatgacagtaacaaccacattgatacagaaaatgtaatggcaaacagtacttccttaccaatttcagccgtcgagtctcgttttgagaaaaccttagacactcaattagagattgtcatagaaattttgcacaaagaatatgatcaatttataaattccattagattgaaattcgagaaatcgcaaaaattagaaaaaataatagcgtccaaactaaatgagcagtctcatctactagattccttagaattggaagaaaattctagttccgttatagaaaaacaagatcatttgatttcccaactgaaggaaaagatcgaatcgcaatctgttttgataaacaatttggaaaaattgaaggaagacatcattaaaatgaaacaaaatgaaaaagttttaaccaaagaactggaaactcagaccaaaatcaataaactaaaggaaaataattgggacagctacatcaatgacttggaaaaacaaatcaatgaccttcaaatcgataaatcagaggaattccacgtaatacaaaatcagttagacaaattagacttggagaattaccaattaaaaaatcagttaaatactttggacaaccaaaagttaatactatcccaatatgaaagcaattttatcaaatttaatcagaaccttttactacaccttgacagtattttcaatattctgcagaaaatcttacaagaaagttctattgcccaatttgacaggaaaatgaaatccataaaatctgtaccaaatgcgttgaaaaacttgaatttgattcagcccaaactagaatcgctctacacttttatagagactgcattagagtccataataaactcatatatctcttcactgatttccatggaaaccccagagcagccacatcaacagggcaatgaattaacggcaactccaaataaagaactgaccttacgaatagaggagttacaaagaagatggatatccgaaagagaaagacggaagctcgatgctaacgcttcagaagctcgaatcaaggctttagaacaggaaaatgagtcattgagatcgaaacttttcaacctatcaatcaacaatccctaa

SEQ ID NO: 125 - DNA, 1272 bp, Saccharomyces cerevisiae
atggcggaaatacctgatgaaaccatccagcagttcatggcattgaccaatgtgtcgcataacatagccgttcaatatctctctgaatttggagatttaaatgaagcactaaattcctattatgcttctcaaacggatgaccaaaaggatagaagagaggaagcacattggaacagacagcaggagaaggccctcaagcaagaagccttctccaccaactcttcgaataaagccataaatacggagcacgttggtgggttatgtccaaaaccaggatcctcacaaggtagcaacgagtacttgaaaaggaaaggttctacctctcctgaaccaaccaagggtagtagccgctctggaagtggtaacaactccaggtttatgagcttttcggatatggtaagaggtcaagctgatgatgacgatgaagatcaaccgagaaatacttttgctggtggtgaaacatccggcttagaggttacagatccttcagatcctaattcattactgaaggatttgctggaaaaagcgagaaggggtggtcaaatgggcgctgaaaacggattccgtgatgacgaagaccatgaaatgggtgccaataggtttactggaagaggttttagattagggtcaaccatcgacgcagcagatgaagtcgtagaagacaacacttcacaatcacaacgtagaccagaaaaagtcacaagagaaattacattttggaaggaaggttttcaagtggccgatggtccgctttatcgctatgatgatcctgcgaacagtttctatttgagcgagttaaatcaagggagggctccattaaagctcttagatgtgcaatttggacaagaagttgaagttaatgtatataaaaaattagatgagtcttataaagctccgacgagaaaactgggcggtttttcaggccagggccaaagactaggatctcctatcccgggtgaatcgtcacctgcggaggttccaaagaatgagacacccgctgctcaggaacaacccatgccggacaatgagccaaaacaaggcgacacctccatccaaattagatacgcaaatggcaaaagagaagttttgcactgcaattccacagatacagtaaagtttttgtatgagcatgtgacatcaaatgcgaacactgacccatcgaggaatttcaccttgaattatgcctttcctatcaaaccaataagcaacgatgagacaacattgaaggacgctgatctgctgaactccgttgtcgtgcaaagatgggcatga

SEQ ID NO: 126 - DNA, 249 bp, Saccharomyces cerevisiae
atgtcaacatatgacgaaatcgaaatcgaagatatgacgtttgagcctgaaaatcaaatgttcacctatccttgtccctgtggagataggtttcaaatatatctggatgacatgtttgagggcgaaaaagttgctgtttgtcccagctgctcactgatgatcgatgtagttttcgataaagaagacttggctgagtactacgaagaggcaggcatccacccccctgagcctattgccgctgctgcctaa

SEQ ID NO: 127 - DNA, 663 bp, Saccharomyces cerevisiae
atggcttctagagtggacgaaactacagtcccctcatactactattacgtggatccggaaactacatatacgtaccaacaaccaaatcctctacaggacttgatatcggtgtatggcttggatgacatctccaggcaagtggcaagaacaaatttggacggcactaaagccgtgaagctaagaaaatcttacaagaaccagatagcagatctttcaggtaaattctccaccataccgaccagagaaaatggtaaaggtggtcaaatagcacatattcttttccaaaataacccagacatgatgatacaaccacctcagcagggtcaaaacatgtcagagcaacaatggcgcgaacagctgcgcaatagagacatagcattgtttcagcctccaaatttcgattgggacctttgctcttcggtactatcgcagtttgaaaggtcatatccaagcgagttcgcaaaccagaaccaagggggagcccaagcgccgtttgatatagacgacttggcgtttgatctagacggtacaggaaaaagccaatccggctcaaattcaggtaacaatagtaagaaaaggaagaacaaatctagtggaagttcgatggctacaccaacacatagtgacagtcatgaggatatgaaaagaaggaggctggagtag

SEQ ID NO: 128 - DNA, 3498 bp, Saccharomyces cerevisiae
atgaccggcttgaatggagatgatcctgatgactactatctgaaccttaatcaagatgaagagtctctacttaggtcaagacacagtgtcggctcaggagcacctcatagacaaggctctttagtgcggcccgaaagaagccgactgaacaatcctgataatccacatttttattatgcgcagaaaacgcaggagcagatgaatcacctggatgttttaccatcaagtaccggtgtaaacccaaatgcaactcgtcggagtggctccctgcgctccaaaggctcagtgagaagcaaatttagtggccgcgaaacggatagctatcttttacaagatatgaatactactgacaagaaggcttccgttaaaataagtgatgaaggtgttgcggaagacgaatttgataaagatggtgatgtggacaatttcgaagaaagctccacgcagcccataaataagtctatcaaaccattaagaaaggaaacgaatgatacattgtcattttggcagatgtactgttatttcattacgttttgggcacctgctccaattcttgctttctgcgggatgccaaagaaggaaagacaaatggcgtggagagaaaaggttgctttaatttctgtcatcttgtacattggtgcgattgtggctttcctgacttttggtttcactaaaaccgtttgtagtagttcgaaactacgtttgaaaaacaacgaagtatcaacagaatttgtcgtaattaacggtaaggcttatgaattggatacttcctcgcgttccggtatacaagacgttgaagtagattcagacaccctttatgggccctggtcagatgctggtaaagatgcttcgttcttgtttcaaaatgtgaatggtaactgtcataaccttataactccaaagagtaattcttccattccccatgacgatgataataatttagcatggtattttccttgtaagttaaagaatcaagatggctcttcgaagccaaacttcacagttgaaaattacgcaggatggaactgtcatacgtctaaagaagatagggacgcattttacggtttaaagtcgaaagctgatgtttacttcacttgggatggtataaagaactcttctagaaacttgattgtttataatggcgacgttttggatttagatcttcttgattggttggaaaaggatgacgttgactatcccgttgtattcgatgacttgaagacttcaaatttacaaggttatgatctttcgttggttttgtcaaatgggcatgaaagaaaaattgcgagatgtttgagcgaaattattaaagttggtgaagtagactccaaaaccgtcggttgtattgcctctgatgtcgttttgtatgtttctctggtatttattctttcagtggtgataattaaattcataattgcctgctacttccgttggactgtagctaggaaacaaggtgcatatatcgtggacaataaaacaatggataaacacacaaacgatatcgaggattggtctaataatattcaaacaaaagctcctctaaaggaagtagatcctcatttgaggccaaagaaatactcaaaaaagtcgttgggacacaagcgtgcttcaacctttgacttgctgaaaaaacacagctccaaaatgtttcaatttaacgaatctgtgatagatctagacacctccatgagcagttcactacaatcttctggttcatacagaggaatgacaacaatgaccactcaaaatgcttggaaactctcgaatgaaaacaaagctgtacattcccgtaatccatctactttgttgcctacatcctcgatgttttggaataaagcgacttcctctcctgtaccaggatcatcgctgattcagagtcttgattcgacgattatacatcccgatatcgttcaacaaccaccactggattttatgccatacgggttcccattgattcatactatctgttttgttacttgttattctgaggatgaagagggtttaagaaccactttagactctctttctaccacagattatccaaattcccataaactactgatggttgtttgtgatggtttaattaagggctcgggcaacgataagactactccagagatagcgttaggaatgatggacgactttgtcaccccacctgatgaagttaaaccttactcctatgtggcagtggcatcaggctctaaaagacacaatatggccaagatatatgcgggtttttacaaatatgacgattctacaattccaccagaaaatcaacaacgtgtcccaatcattacaattgtgaagtgcggtactcctgcagagcagggggccgccaaacccggtaacagaggtaagcgtgattctcaaattattctgatgtcctttttagaaaaaataacatttgatgaaagaatgactcaattggaatttcagcttttaaaaaatatttggcagattacggggctaatggcagacttctacgaaacggtacttatggttgatgctgatactaaagtctttcccgatgctttaactcatatggtcgctgaaatggttaaagatcctttgattatgggtctttgtggtgagaccaagatcgctaataaggcacaatcttgggtaactgcaattcaagtgtttgagtactatatttcgcatcatcaggctaaagcttttgaatctgtcttcggttcggtaacttgtttgccgggatgtttctcaatgtatcgtataaaatctcctaaaggttcagatggttattgggtacctgtattggcaaatccagatattgttgaaagatattcggataatgttacaaacactttgcataagaagaacttattattacttggtgaagatagatttttatcttcattaatgttaaagactttccctaagagaaagcaagtatttgttccaaaagctgcttgtaaaactattgcccctgataaattcaaagtcttactttcccagcgtcgaagatggattaattctacggtacataacctttttgaattagttctaatcagagacttatgtggcactttctgtttttccatgcaatttgtgattggtattgaattgattggtactatggtactgccgttagccatttgctttactatttatgtcattatttttgccattgtatcaaaacctacacccgtaatcactttagttttactggcaattattcttggtctgcccggcttaattgttgttataactgctacgagatggtcgtacctatggtggatgtgcgtatatatttgtgctttgcctatttggaatttcgtactaccttcatatgcgtactggaaatttgatgacttctcatggggtgatacgagaactattgcgggaggtaataaaaaggcacaagacgagaatgaaggtgaatttgatcactcaaagattaaaatgaggacatggagggaatttgaaagggaagatattctcaatcggaaggaggaaagtgactccttcgttgcatag

SEQ ID NO: 129 - DNA, 3999 bp, Saccharomyces cerevisiae
atgactgaaagaataccaataaagaattatcaaagaacaaatgccaaagctttacttaaattgactgaaaaactttttaacaagaacttttttgatctctatttaacctctcagcaattggtcgttcttgaatacctgctgtcgatttcaagtgaagaagacaaactgaaagcatgggactatttcttaaagggaaacatagcattaaatgtcgaaaaatcatttccattaacccaagaagaagaacatcacggagcggtctctcctgccgttgacacacgatcagatgatgtatcatcacaaacaattaaggacaataacaatactaataccaacaccagtatcagcaatgaaaatcatgttgaaaatgaaattgaagataaaggcgataacgcaatagcaaatgaagataattttgtgaataatgacgaaagtgataatgttgaagaagacttattcaaattagatctagaggacttgaagcagcaaataagcggaacaaggtttattggaaacttatccttgaaaatcagatacgtcttgtggcagtgcgccatagattatatatactgtgatcgtaatgagtttggtgatgaaaatgatacagaatacaccctattagatgttgaagagaaggaggaagaggaaattggtaaaaatgagaagccacaaaacaaagaaggtatttcgaagttcgccgaggatgaagattacgacgatgaagacgagaactatgatgaagacagtacagacgtaaaaaatgtcgatgatcctccaaaaaatctcgattctatttcctcttctaatatcgaaattgacgatgaacgacgcttggtgctaaatatctcaatatcaaaagaaacactgtcaaagttaaaaacaaataatgtagaagaaattatgggaaattggaacaaaatttaccacagttttgaatacgataaagaaactatgataaagcgattaaaacttgaagaaagcgataaaatgatagagaaaggaaagaagaaacgaagtcgaagtgatttagaagcagctaccgatgaacaagatcgcgaaaatacaaatgatgagccagatactaatcaaaaattgcccactcctgaaggttcaacattcagcgatactgggaacaagcgccccaaacaaagtaatttagatttaacagtcaatctaggcatcgaaaatttatcattaaagcaccttctatcatctatccagcaaaaaaaatcccaattaggaatatcagattacgaattaaaacatctgattatggatgtcagaaaaaatcggtcaaaatggacatcggatgaaagaattgggcaagaggaattatacgaagcctgtgaaaaggttgttttggaacttagaaactacactgagcattctacaccatttctgaataaagtgagcaaaagagaagcccccaattatcatcaaatcatcaaaaagtccatggacctgaatactgttttaaaaaaactgaaaagctttcaatatgactccaaacaagaatttgtagacgatattatgctaatatggaaaaattgtttgacctataattcagatccttcacattttttgagagggcatgctattgctatgcagaagaaatctcttcagttgattcgcatgattccaaatatcacaatccgaaacagggctgatttagaaaaggaaattgaagatatggaaaaagacaaagactacgaattagatgaggaagaggaagttgctggttctggaagaaaaggattgaatatgggagctcatatgttggccaaagagaatggcaaggtgtcagaaaaagatagctctaaaaccgtcaaggatgaagcaccaaccaatgatgacaaactaacttctgtcatccctgagggggaaaaagagaaagataaaactgcttcatctactgtaacggtacacgaaaatgtaaataagaacgaaataaaagaaaatgggaaaaatgaagagcaagatatggttgaggaaagtagtaagactgaggattcatcaaaagatgctgatgctgccaaaaaggatacggaagacggactacaagataaaactgcagaaaataaggaggctggggaaaataatgaagaggaagaggatgatgatgacgaagatgaagacgaagacatggtcgactcccaatcttatttacttgaaaaggatgacgatagagacgatttggaaatatccgtgtggaaaactgtaactgccaaagttcgtgcggaaatttgcttaaaaagaactgaatattttaaaaatggaaaattaaatagtgattcagaggcgtttttgaaaaacccacaaagaatgaaaaggttcgaccagctttttcttgaatataaagagcagaaagctttagaatcatatcgtcaaaaaatagagcaaaattccattatgaaaaatggctttggaacagtactaaaacaggaagacgatgaccaattgcagtttcataatgatcactctttaaatggaaatgaagcttttgaaaagcaacccaatgatattgagttagatgataccagattcctacaggaatatgatattagtaacgccattcctgacatagtatacgagggagtaaatactaaaacattagacaagatggaagacgcttccgtggaccgcatgcttcaaaatggtatcaacaaacaaagcagatttctggctaacaaggatttaggactaacacctaaaatgaaccaaaatatcacactgattcagcaaattaggcacatatgccataaaatatccctgatcagaatgttacagagccctttatcggctcaaaactccagaagcaatcccaacgctttccttaacaaccacatttataattacactattattgatgactcactcgatattgatccggtgtcacagcttccaacgcatgattacaaaaacaacagggagctgatatggaaattcatgcataagaacatatctaaggttgctatggccaatgggtttgaaactgcccatccatcagcaataaacatgcttactgaaatcgccggggattacctatctaatctgataaagactttgaagcttcatcatgaaactaactccttaaatagaggaacaaatgtggaaatgctgcaaacaacactgttggaaaacggtatcaacaggccagacgatctattttcctatgttgaatctgaatttggtaaaaaaactaagaaacttcaggacatcaaacagaaactagaaagctttttgagagccttattaaggccaactttgcaggagttgtccgagagaaactttgaagacgagagccaaagcttttttacaggtgactttgccagcgaattgactggtgaagacttctttggttttagagagcttggattagaaaaggagtttggagttttgagttcatctgttccattacagttactgactactcagtttcaaactgttgacggggaaaccaaagtgcaggccaaaaagatccaaccggaagaatcagacagcattgtgtataagaaaattacaaaaggtatgctggatgctggttcattctggaatactctacttcccctattacaaaaagattatgaacgttccaaggcctatatagcaaagcaaagcaagtcatctgcaaatgataaaacctcaatgacttccacagaagacaattctttcgctttactagaagaggatcagtttgtctcaaagaaaaccgcaacgaaggcaagattacctcctactggtaagataagtaccacatacaaaaagaaaccgatcgcaagcgcgtttatacttccagaagaagacttggaaaacgacgtaaaagcggatccaacaacaactgtaaacgccaaagtgggtgcagaaaatgatggagattcttccttatttttgcgaacgcctcaacctttagatcctttggatatggatgatgcttttgatgataccaatatgggcagcaatagttcatttagcttgagccttcctcgccttaatcaataa

SEQ ID NO: 130 - DNA, 2901 bp, Saccharomyces cerevisiae
atgaatccgggcggtgaacaaacaataatggaacaacccgctcaacagcaacaacaacagcaacaacaacagcagcaacagcaacagcaggcagcagttcctcagcagccactcgacccattaacacaatcaactgcggaaacttggctctccattgcttctttggcagaaacccttggtgatggcgacagggccgcaatggcatatgacgccactttacagttcaatccctcatctgcaaaggctttaacatctttggctcacttgtaccgttccagagacatgttccaaagagctgcagaattatatgaaagagcacttttggtaaatcccgaactatcagatgtgtgggctactttaggtcattgttatctgatgctggatgatctgcaaagagcttacaatgcctatcaacaggctctctaccacctcagtaatcccaacgtaccgaaattatggcatggaatcggcattctttatgacagatatggttcgctcgactatgccgaagaagcttttgccaaagttttggaattggaccctcattttgaaaaggcaaacgaaatttacttcagactaggtattatttataaacatcagggtaaatggtctcaagctttggaatgcttcagatacattctccctcaacctcctgctcccttgcaggagtgggacatatggtttcagttgggtagtgttttggagagtatgggagagtggcaaggtgcgaaggaagcctacgagcatgtcttggctcaaaatcaacatcatgccaaagtattacaacaattaggttgtctttacggtatgagtaacgtacaattttatgaccctcaaaaggcattggattatcttctaaagtcgttagaagcagatccctccgatgccactacatggtaccatctcggtagagtgcatatgattagaacagattatactgccgcatatgatgctttccaacaagctgttaatagagattcaagaaaccctatcttttggtgctcaatcggtgttttatattaccaaatttctcaatacagagacgccttagacgcgtacacaagagccataagattaaatccttatattagtgaagtttggtacgatctaggtactctttacgaaacttgtaacaaccaattatctgacgcccttgatgcgtataagcaagctgcaagactggacgtaaataatgttcacataagagaaagattagaagctttaacaaagcagttagaaaacccaggcaatataaacaaatcgaacggtgcgccaacgaatgcctctcctgccccacctcctgtgattttacaacctaccttacaacctaatgatcaaggaaatcctttgaacactagaatttcagcccaatctgccaatgctactgcttcaatggtacaacaacagcatcctgctcaacaaacgcctattaactcttctgcaacaatgtacagtaatggagcttcccctcaattacaagctcaagctcaagctcaagctcaagcacaagctcaagcacaagcacaagctcaagcacaagcacaagcacaagcgcaagcacaagcacaagcacaggcgcaagcacaggcacaagcacaagcacaagcacatgcacaagcgcaagcacaagcacaagcacaggcacaagcacaagcacaggcgcaggcacaacaacaacaacaacaacagcaacaacaacaacaacaacaacaacaacaacaacaacaacaacaacaacaacaacaacaacagcagcagcaattacagcccctaccaagacaacagctgcagcaaaagggagtttctgtgcaaatgttaaatcctcaacaagggcaaccatatatcacacagccaacagtcatacaagctcaccaactgcaaccattttctacacaagctatggaacatccgcaaagctctcaactgccacctcaacagcaacaactacaatctgttcaacatccacaacaacttcaaggccagcctcaagcccaagctccccaacctttaatccagcataacgtggaacagaacgttttacctcaaaagagatacatggaaggtgcaatccacactttagtagatgccgccgtatccagtagcacccacacagagaataacacaaagtctcctcgtcaaccaacccatgccattccaacgcaagctcccgcaacaggaataacgaacgctgaaccacaggtaaagaagcaaaagttgaactctccaaattcaaacatcaacaaattagtaaatactgctacttccattgaagaaaatgcaaaatctgaggtgagcaaccaatcgccagcagtagtggagtctaataccaataatacttcacaagaagaaaaacctgtaaaagcaaactcaataccttcagtaattggcgcacaggaacctccacaggaagctagtcctgctgaagaagctaccaaagcagcttctgtttctccttctacaaaaccgcttaatacggaaccagagtcatctagtgtccaaccaactgtatcatcagaaagttcaacaacaaaagcaaatgaccaaagcactgctgagaccatagaactttctactgctactgttcctgcagaagcaagccctgtagaagacgaagtaagacagcattctaaagaggaaaacggcacaactgaagcatctgcaccttctactgaagaggcggagccagcagcttccagagatgctgaaaaacaacaagatgaaaccgctgctacaacgataactgtaatcaaacctactttggaaacaatggaaacagtgaaagaggaggccaaaatgcgtgaggaagagcaaacatctcaagaaaaatccccacaggagaacacacttccaagagaaaatgtagtaaggcaagtggaagaagatgaaaactacgacgactaa

SEQ ID NO: 131 - DNA, 1182 bp, Saccharomyces cerevisiae
atgagtagcagtggtgtttatacgtgtaattcatgtgtcctgacctttgattcaagcgacgagcagcgggcccacatgaagtccgattggcatcgctacaatttgaaaagacgtgttgcacaattgccaccaatatcatttgagacatttgactccaaagtgtctgcagctgctgccagtactagtaagtctgctgaaaaggagaaacctgttaccaaaaaggagttgaaaagaagggaaaagcaagcattgctcgaaaagaaaaagaaactgttggaaattgccagggctaatatgcttgaaaacatgcaaaagagccaagagggaaatactcccgacctaagcaagctctctttgcaagaaaatgaggagaacaaggaaaaggaagaacctaagaaggaggagcctgaacagttgaccgaggaagaaatggcggaaagagtaatgcaagaaaacgtacgcaacagagtcgatattccactggaacaatgtctattttgtgagcacaataagcacttcaaagatgttgaagaaaacctggaacacatgtttaggacccacgggttttatatcccagaacagaaatatctagtcgacaagatcggcttggtaaaatacatgtcggagaagattggtcttgggaacatttgtattgtttgtaattaccaggggagaacgttgaccgctgtaagacagcacatgttggcaaagagacactgtaaaattccctacgaaagcgaggatgaaaggttggagatatctgaattctacgattttacaagctcatacgcaaactttaatagcaacacaacaccagacaacgaagatgactgggaagacgtgggcagcgacgaagccggaagcgacgacgaagatctgccacaagagtacttatataacgatggtatagagctgcatctaccgacaggcatcaaagttggccacaggtccttgcaaagatactacaagcaagacttaaagcccgaggtgatactgaccgaaggccaaggtaccctggtcgctgcagagacgagatcgttcttacctgccttcgacaaaaagggcgtgcagactcaacagcgtgtttggcaaactgagaggttcgacaagaaaaggctcgataagagaagtgccaagttcgtcaataaccaaccacactacagagaccaacttctgcagtaa

SEQ ID NO: 132 - DNA, 1338 bp, Saccharomyces cerevisiae
atgtccaaaaaacaggaatatattgcaccaataaaataccaaaacagcttgcctgtgccacaattacctcctaaacttctggtttaccccgagtctccggagacgaatgcagactcctcccaactaatcaattcactatacatcaaaacaaacgtcactaatctaattcaacaggatgaagatttaggtatgccggttgatttgatgaaattcccaggcctattgaacaaattagattctaaactgctttacggctttgataatgtgaaattggacaaagatgatcgaattttactgagggaccctagaatagatagactgaccaagactgatatatcaaaggttaccttcttgagacgcaccgaatatgtctccaatacaattgcagcccatgataacacatcgttgaaaaggaaaaggcgcttggatgatggagattcggatgatgaaaaccttgatgttaatcatataattagtagggtcgaaggaacattcaataagacggacaaatggcaacatccggtgaagaagggtgtcaagatggttaaaaaatgggacctattgcccgacacagcttcaatggaccaggtgtattttatcctaaaattcatgggcagtgcatcattggatactaaagaaaaaaaatctttaaatacaggaattttcaggccagtggaactggaagaagacgaatggatttccatgtatgccacagatcataaggactctgccattctggaaaacgaattggaaaagggtatggatgaaatggatgatgactctcatgaaggtaaaatatacaagtttaaaaggataagagactacgacatgaaacaagtggctgaaaagccaatgacagagttagctatacgtttgaatgataaggacggcatagcttactacaaacccctacgttcaaagatcgaattaagacgtaggagagtaaacgacatcattaaacctttggtgaaggagcacgatatagaccaacttaatgttactttgagaaaccctagcactaaggaagctaacataagggacaagttaagaatgaagtttgaccccataaatttcgccactgtagatgaagaagacgatgaagacgaggagcaaccagaagacgttaagaaagaatcggaaggcgattcaaaaacagaaggttctgagcaagaaggagaaaatgagaaggatgaagaaatcaaacaggaaaaagagaatgaacaagatgaagaaaataaacaagatgaaaaccgtgcagctgatacgcccgaaacttcagatgctgttcatactgaacaaaaaccagaggaagaaaaggaaactttacaagaagaatag

SEQ ID NO: 133 - DNA, 2718 bp, Saccharomyces cerevisiae
atgaataatcagccgcagggtaccaacagcgttccaaatagtattggaaatatatttagcaacattggaactccatcttttaacatggcgcaaattccgcaacagctgtatcagagcctcacaccacaacaattgcagatgattcagcaacgacaccaacagttactgaggagtcgtctacaacaacaacaacaacaacaacaacaaacttcaccgccaccgcaaacgcatcaatctccaccccctcctccgcaacaatctcaacccattgctaatcaatcagcgacttctacccctcctcctcctccagcaccacacaacttacatccccaaattggtcaagtgcccttagctccagcgcctattaatttgcctccacaaattgctcagttacctttggctacacagcaacaagttttgaacaagttgaggcagcaggccatagcaaaaaataatccacaggttgtgaatgcaattactgttgcacaacaacaagtgcaacgccaaattgagcagcaaaagggacagcaaacggcacaaactcagctagaacagcagaggcaattgctggttcagcagcaacagcagcagcaacttagaaaccaaatacagcgacaacagcaacaacagtttaggcatcatgtgcaaatacaacagcagcaacaaaagcaacaacaacagcagcagcagcatcagcaacaacaacaacaacaacagcaacagcagcaacagcaacagcaacagcagcagcaacaacaacagcaacaacaacaacaacagcagcagcagcagcagcagcagcaaggacaaataccgcaatctcagcaagttcctcaagttagatccatgagtggacaacctcccaccaatgttcagcccactattggccaacttcctcaacttccaaaattaaacttacccaagtaccaaactattcaatacgatccaccagaaaccaagctaccatatccaacctattggtcagacaaaaaagcagatacggatactttgttgtacgaacaaattatccagcgtgataaaattaacaaatattcgctaataagagaaaccaatggttacgatccgtttagcatttatggatttagtaataaagagtatattagtagactgtggcatacactgaagtattatcaagatttgaagaacactagaatgaaatctatcacaagcacttctcagaagattccttcggcaagtatttggggaaatggttactcagggtatggtaatgggattacgaatacaactaccagagttattccacaagtagaagttggaaataggaagcattacctagaggataaattaaaagtctataaacaggccatgaatgagacatcggaacagttagttcccataagattggagttcgatcaagatcgtgacagattcttcctcagggacactttgttatggaacaaaaatgacaagcttattaaaattgaagactttgtggacgacatgttgcgagattaccgatttgaggacgctacgagagagcaacacattgatactatttgtcaatctatacaagagcagattcaggagtttcaaggaaatccatatatagagttgaatcaggaccgtctaggcggtgatgacttgagaattagaatcaagctggatattgtcgtgggacaaaaccagttaatcgatcaatttgagtgggagatctctaatagtgataactgtccagaagagtttgcagagtccatgtgtcaagaattagaactaccaggtgagtttgtgactgccattgctcactccataagagagcaagttcatatgtatcataaatcactggcactgttaggttacaattttgatggatcagcgatagaagatgatgacattagaagcagaatgctcccaacgattactcttgatgatgtttataggcctgcagcggaaagcaaaatttttactccaaacctattacagatttcagctgcagagttagagagattggataaagataaggacagagacacaagaaggaaaagaagacaaggtagatctaatagacgtggtatgctcgcattgtccggcacatctgcaagtaatacatctatgaacggcgttcacaacacagtagcagcaggaaatgcttcatcgttgccaccaggagagattttactgccagatattgcagatattccaagaactttcaggactccagtacctagcactttaatgcctggtggtgttgacgtaggcccttctgtggaatcgtacgaattgagaaacacaaccacttataaaagcaggccagatagacctaagccagtttcacctccttgttatattattgaccatattccgggtcattcgctactactttctattaaactgcctgggaaagttaatacaaaagaagagttcgcagcagcgcccaatgacacaagtagtggcaccaatgcaatgcttccgagtccagaatcgctgaaaactaagctgaatagtaacattcgcgctggtgtgacgataccttcaatcccaaacccgattgccaatcacactgttactaattcacccaatcccacactgcagccagtaatcccaggtggggcagctagtaaatcggtacctacacctagtcttcctatagcacctccagtagcaccacatgatagcgaagcgacattgttgactaatagcaataatggtagcagtaacaataacacacagaatacatag

SEQ ID NO: 134 - DNA, 237 bp, Saccharomyces cerevisiae
atgtcacagaacaacacaatcagctcaatgaatcctgaaagagcttacaacaatgtaacgctgaaaaatttaacagcatttcagttattatctcaaagagaaaacatatgcgagttattgaacttggtagaaagtacggaaagacacaatagtattatcaatcctgaaaggcaaaggatgagtttggaagaaatgaaaaaaatgctcgatgctttgaaaaatgaaaggaaaaagtag

SEQ ID NO: 135 - DNA, 1074 bp, Saccharomyces cerevisiae
atgaggttagctgtggttgtgaccctacttgttcactgtttcctagtgacatgctctccaggagataatttagatgagtttatagactgtacgtatgcgtgcgagtataacagaagatgtccgaattcccaaataaactacattgaccctgaaaccaacatgtttcatgatattgagtttttcgataccccgcctttgtactctaagttattgttctgggattgtatctcagattgtgattaccaatgtcagcacatcattacgcgctggagaattgacgaagaggaagaaatataccaattccatgggaaatggccatttttgagagtattggggactcaggaattcttctcgacgatattcagtataggtaactttattccacattataagggatttgtaaagttttctagaatcatacgcgaagagggagataggaggagaaaaaacagcagaagtatactgatttggaactacctttacgttactgtggcgggaatgttggcttggacagcaagctcggtctttcactgtcgtgatttgatcataacggagaagctagattactttttcgcaggtttaactgtcctaacagggtttcatgcaatatttgcaagaatgacttccatgttcctgtaccccaagatagcacaagcgttcactgcgtcagttgcggcaatctttgccctgcacatcttgagactctatgttgactggtcgtacacatacaacatgagattcaacatttttttcggtgttttacagtatattttattgataatgttatcatgccaaaactaccatgctctgcaaaagcaaaagctaatgggcgaattcaagaaaaccgcgtactccagcttcaagcggcaaatcttcaaactgtgtgtcattccaattcttcttgtgattgtaaccacaatggccatgtcattggaactgtttgacttttttagttacgaatggcagattgatgcccatgcgctatggcatctgtgcacaatatggccctcgtgggtattgtacgactttttcctcgaggattatgcctattggggtaatagacaattgtactaa

SEQ ID NO: 136 - DNA, 1176 bp, Saccharomyces cerevisiae
atggtttcattgttcaaaagaggtaaggctccaccgctcacgaaagaaggccccacttctaaaaagcctcctaacacagcgtttagacaacaaaggcttaaggcatggcaaccaatactgtctcctcaaagtgtgcttccgttgttaatattcgttgcatgtatatttactcctattggtattggactcattgtaagcgctactaaggtacaagatctaacaattgattatagtcattgtgatacaaaagcatctacaactgcttttgaagatataccaaagaagtacattaaatatcactttaaaagtaaagttgaaaataaaccacaatggaggctaaccgaaaatgaaaatggcgaacaatcatgcgaactgcagttcgaaatcccaaacgatatcaagaaatccatttttatatattataaaataaccaatttttatcaaaatcatcgcagatatgtccaatcgtttgacacaaagcaaatattaggggagcctatcaaaaaagatgatctggatacaagctgtagtccaataagaagtagggaagacaaaataatatatccctgtgggttgatcgctaattccatgtttaatgatacattttctcaggtgttgagtggtatagatgacacagaagactataatttaactaacaagcatatatcatggagtattgatcgtcacagatttaaaaccaccaagtataatgctagcgatattgttccaccgccaaactggatgaagaagtatcccgatgggtatacagatgaaaatcttcctgatatccatacttgggaagagttccaggtatggatgaggactgcagcctttcccaagttttacaagttgacgttgaaaaatgaatctgcttctttaccgaagggtaaatatcaaatgaacattgagttgaattatccgatttcactctttggtggcacaaaatcatttgtactgactacaaatggagctattggtggtagaaatatgtcactaggcgtactgtacctcatcgttgcagggctttgcgccttatttggcatcatttttttggttaaattaatcttccaaccaagagcgatgggtgatcacacttatttgaattttgatgatgaagaaaacgaggattatgaggatgtacacgcagagaatacaacattgagggaaattttatag

SEQ ID NO: 137 - DNA, 1860 bp, Saccharomyces cerevisiae
atgcatagcgatactaacggtcgaacaaagagcaataatagcccatcagacaataatccaaatgagacagttatattaatagatagtgataaggaagaagacgcttctattcgcgaggctaaccttcctgtgaggttgtacccagacagacgggtagggagacgacgtgatgctttaaataggtttgtgaggtcagattcaaggagcaggaattctcaacggactcacataacagcaagctctgagcgccctgattttcaagctaataacgacgatatcactataatacgagaagttggacggttttttggagacgatggacctattgatccttctgcacattacgtcgatcttgatcaggagccaggttctgaaacgttagaaactccaagaacaatacaagtagataacacaaacgggtacttgaatgataatggtaataacaatgaaagtgatgatggcctgacaatagttgaagagagaacaacacgacccagagtgacactgaacctgccaggcggggagaggcttgaggtaactgcgacgacaacagacataccgataagaaggtcatttgaattccaagaagatctaggcgcatcacgcaggcagttactgagaaggagtgctacaagagcccgtaatttatttgtggaccggtctgatgaaaacgatgaagattggacagatgatacccataatttaccagaagctattcaaagggctcgtagggagagccgtatgcgaatgagcagacggattgcagaaagacaacgtagggtacaacagcaaagggtgtccagtgacgaaaatataagtacatctattaggttacaatcaatcagggaacgaatacaatcatatacgcctgatattcgtagtgcttttcatcgtgcagagtcactgcatgaattcaggtctattttacaaaatgttgctccaattactttgcaagaatgtgaagaagaactaatggcactcttcactgagttcagaaatcaactgctgcagaattgggccattgatagggtcagaaatactcaggaagaggccttgagacttcaccgagaagcattggaaagacaagaaagaacggcaggaagagttttccatcgtggcacattacgtgaatcaatcacaaactatcttaatttcaatggagaggatgggttcttaagtcgtttgtggagtggcccagcattgagcgatgctgacgaggaacgacacactcaaaatatcattgatatgatccaagagagagaagaacgagagcgggatgtggtcatgaagaatctaatgaacaaaaccagagcccaacaggaagaattcgaagctagggcagccagcctacccgaaggttatagcgcgtcatttgatactacgccaaaaatgaaattagacattacgaaaaatggtaaagaagaaaccatcattgtgacagatgacgatttagcaaaaacactcgaggatattcctgtttgttgcttgtgtggcgcagaattgggggtcggaataccggatgattttactggaatcagtcaaaaggatcgtggtgtttcatttgaagggttagtgtcaaaatacaaattccactgcccctaccaaaccctagccagaccatcaatgcttgatagagatttatcaaaacgaacgtttattgcatcgtgtggtcatgcattttgtggtaggtgtttcgccaggattgacaatgcaaaaaaaaaatctaagatgcccaaaaagaaactagctcaactaaagggttcggctcatccggataattatggccccaaattatgtcccgcagattcttgtaagaagcttattcgttcaagaggcaggttgaaggaggtttacttctag

SEQ ID NO: 138 - DNA, 321 bp, Saccharomyces cerevisiae
atgatttcagtcaatttttcaccattgatctcttcttcgaacacccacatttgtactattggagcgaaaacttctgggtatcccttacaattttcggcaaccaccttggcaatagtagtaccccagttaccagatccaatcacagtaaccttgaaaggcttttcggcagccttcaaagaaacagaagaggaacttctctttctaccagcattcaagtggccggaagttaagtttaatctatcagcagcagcagacatctttatattatcaatatttgtgtttgtggagggggggggtgtacaatatacaattgtttcttga

SEQ ID NO: 139 - DNA, 342 bp, Saccharomyces cerevisiae
atggccggtttgaaagacgttgtcactcgtgaatacaccattaacttgcacaaaagattgcacggtgtctccttcaagaagagagctccaagagctgtcaaggaaattaagaagttcgccaagttacacatgggtactgatgatgtccgtctagctccagaattgaaccaagctatctggaagagaggtgtcaagggtgttgaatacagattaagattgagaatttccagaaagagaaacgaagaagaagacgccaagaacccattgttctcctacgttgaacctgtcttagttgcttctgccaagggtctacaaactgttgttgtcgaagaagatgcttaa

SEQ ID NO: 140 - DNA, 1680 bp, Saccharomyces cerevisiae
atgatggaagaattctcgtacgatcacgattttaacacacattttgctacagatttggattatttgcaacatgaccaacaacaacaacaacagcaacaacatgatcaacaacataatcaacagcaacaaccacaaccacaaccaattcaaactcaaaacctggagcacgaccacgaccaacatactaatgatatgagtgcttcatcgaatgcatcagatagtggacctcaaaggcccaagaggactcgcgcaaagggtgaagcactagatgtgctaaagcgtaaatttgaaataaatccaacaccctctttggtagaaagaaagaaaatatcagatctgataggaatgcctgaaaaaaacgtcagaatttggtttcagaacagaagagctaaattgaggaaaaagcagcatggaagtaataaggacacaatcccctcgtcacaatcccgtgatattgccaacgattacgatcgtgggagtacagacaacaatttggtcactacaacaagtacttcatccatatttcacgatgaagacctgacttttttcgaccgtattccgctgaacagcaacaacaactattatttttttgacatttgctcaattactgtgggaagttggaatagaatgaaaagcggcgcactgcaaagaaggaactttcagtctataaaggagttgagaaacctatcgccaataaagattaataacataatgtcgaatgccacagatttaatggttttgatatccaagaaaaactcagaaataaactatttttttagtgccatggcaaataatactaaaattctcttcaggatctttttcccattaagttcagtcacgaattgctctctaactttagaaactgacgacgatataataaatagtaacaacacaagcgataaaaacaatagtaatactaataatgatgatgataacgacgataacagtaatgaagacaatgataatagtagtgaggataagaggaatgctaaggataactttggagaattgaagctaacagtcaccagatcacccacttttgctgtttactttttaaataatgctcctgatgaagatccaaatttgaacaatcagtggtccatatgtgatgatttctcagaaggtagacaggtaaatgacgcatttgttggtggttcgaatattcctcacactttgaaaggtttacagaaatcattaagattcatgaattctctaattctagactataaatcatcgaatgaaatattacctacgatcaatacagcgatccccactgctgcagttccacaacagaatattgcccctccctttctgaatacaaattcaagtgcaacagactcaaatccaaatacaaatttagaagattctctcttcttcgatcatgatctgttatcgagttcgataaccaacaccaacaacggacaaggctctaataatggacgtcaagctagcaaggatgatacgctcaatttactggatactaccgtcaacagcaataacaatcataatgctaataatgaggagaatcatctagcgcaagaacatttatccaacgatgctgatattgttgcaaatccaaatgatcatttgttgtctttaccgactgatagtgaactcccaaatactccagattttttgaagaacactaacgaactaactgacgagcatagatggatatga

SEQ ID NO: 141 - DNA, 1899 bp, Saccharomyces cerevisiae
atgaattcacaaaaggcgagtaaaatgactggctcactaaaaaagaatgatctttatatttttgatttgtctgagcaacttctgaattcattgaaattgatgagctttgactccacactgcgagaagttgaagtagagaagacatctgataatgatagaaacaaggagtctggagatctccaaatagcaagaaagaaggtcacatcaaatgttatgcgttgtagtgtttgtcaaatgagctttgattccagaaatgaacaaaaggcccactaccagactgattatcatttgatgaatgttaagcgtaaccttagggggctggatatcctatccgttgaggaattcgatgcgctgatttctaaggagcatggtatcaagtctgaagacgaaaattctggcggcgagcaaacttcaagcgatcacgaagaaagtgaggaagccagcgatcgggacccggacctacaaactaataattatatggaaactataatcgaaaatgatttgcagaaattagggttccaaaaagacgagtcggatgctattagccatattaatacccaatcgccctacatttatttcaaatcgaagtatcttcaaaagaatgaggtcttggcgatttataagtctctatttaataaaaggagtctttccaaccccaatgaagctttgactttttggaattcgcaagagaacccaatggccatttcagctctgtttatggtgggaggtggacattttgcgggagcaattgtttctcatcaaagactcaatgtcaaggggaacgcgcacaagaaggacgaaaccttaattgagcaagcggtcaatttcttagagcacaagacgtttcacaggtatactaccagaaggaagcaaggtggttctcagtcagcaatggacaatgcaaagggaaaagctaactctgccggttcagcacttcgtaggtacaatgaaagcgctttaaaaacagatatacagggtgtgttgaaggactgggaaccttatctgtcaaagtgtgataatatttttattcgagctcgcaatgtcagtgataaaaaaatttttactgataataccgttctgaacaagggtgatgaaaggataaagagcttcccttttaccacgaacaggcccactgtattagaattgaagaaggcatggtgcgaactttcctacctgaaaatattacctaagcctgaaccattggcagtgaaagaaacagtacaaaagttagaagtgtcgaacaaaaaggacgaatttaaagaaaaacaagaaccgttactggaagaaatccaaacagaagaaataatttctctgctgaagaaaggaagggcaccgttattgatatccttcttaaaaaagaataagcttgatggaaactttcggttaaagcctgagagtaaatattccttaacccctaccatgttacattatgcatcgcagcaagggatgaaacagatggcactgattttgctctcaaatattaaatgcgatccaacgatcaagaacagactaggaaggactgcgtgggatttgaatagaaacgacgacgtgagacacgcgtttcaaattgcaagatacaatcttggagagtctttcactaattgggacgaaactcatatcggtcagccattgagcagagaacaagttgatgaaatcaatgaaaagaaaaaagcaatagagaatgaaaaggcagaaaaactgattaaattggaattagaagccgccaaggaaaaacaaagatttgctaaagatgcagagaggggtccaggcaaaaagttaaccaatattccttcaattcagcaacaaaatttgaattctttgaccgatgaacagagacgcagattgatgagagaacaaagggctagagccgcagaagaaaggatgaaaaagaaatactga

SEQ ID NO: 142 - DNA, 2295 bp, Saccharomyces cerevisiae
atggtgaacgaattagagaatgtgccacgggcgtcgaccttgacgaatgaagagcaaacggttgatcctagcaataacgactcacaggaagatattagtctgggtgattctaacgaaattacttcgttggcatcattaaaggctatccgatcaggaaatgaagaggaaagcgggaacgagcaggtgaatcataatgatgaggccgaagaagatcctctgttaactcgctaccataccgcttgccaaagaggtgatctagcgactgtcaaagaaatgatacatggaaagttgctcgaagttaataacgacggtgactctacggaacatatcactggtttgcattgggcaagtattaataacagactttctgtagtggattttctagtttcacaaggcgctgatgtcaatgcaagggctggtgctttgcatgccacaccattacattgggctgcaaggtatgggtacgtgtacattgtcgattttttgcttaagcatggagctgatcccaccatgaccgatgaccaaggtttcaatctactacacttatctgtgaacagctcaaatataatgctagttctatacgttttattcaacgttgtcagcaaaggtcttttagatatagattgccgagacccaaagggcagaacttcactactatgggctgcttaccagggagattccctaactgtagcagaattgttgaaatttggtgccagcataaagatcgcagatacagaaggattcaccccactgcactggggcactgtaaaagggcaaccacatgtactaaaatacttgattcaagatggtgcagattttttccaaaaaacggataccggaaaagattgctttgctattgctcaagaaatgaacactgtgtactcacttagagaggctctaacacattctggattcgactaccatggttatcccattaaaaaatggttcaaaaaaagtcaacatgctaagctcgttacattcataacgccatttctatttctaggaatcgcatttgctcttttctctcatattaaccctttgtttgtaatcattgtcctttttttattggccattgccactaacaaaggcttgaacaaattcgttctgccgtcatatggacgaatgggtgtccacaacgttaccttgttaaggtcacctttgttatccggtgttttttttggcactttactttgggtgactatcgtttggtttttcaaagttatgcctcgaacattttctgatgaacaatatactaacatcctaatgctggtaatacttgtttctgtattttacctttttgggcagcttgttattatggacccaggatgtttgccagaagaaactgatcatgagaacgtgcgacaaacaatctcgaacttgctcgaaatcggtaaatttgatacgaagaacttttgtatcgaaacttggattagaaaacctctaagaagcaaattttcgcccttaaacaatgctgtggttgcaagatttgatcattactgcccttggatctttaatgatgtcggactcaaaaatcacaaagcatttatattttttattacattaatggaaagtggtattttcacatttcttgcactttgcctggaatattttgatgaacttgaagatgcacacgaagatacatcccagaaaaatggcaaatgtttcatcctaggcgcttcagatctctgttcgggattaatatatgatagatttgtctttctgatattattatgggctttactccaatctatttgggttgcaagtttgatcttcgtccaagcctttcaaatatgtaaaggaatgaccaatacagaattcaatgttttgatgaaagagagtaagtccattggccccgatgggttatctttcaatgaaaacttcaatactactccagaaggatttgcgccgtcgattgatccgggtgaggagagtaatgatactgtactagcaccagtcccaggctcaacgataaggaaaccacgcacttgttttggcgtttgttacgcggtgactggtatggatcagtggttagcggttataaaagaaaccattggcatcaaggatagcaccggacataatgtgtattcgattacatcaagaataccaacaaattacggttggaaaagaaatgttaaagacttttggcttacaagcgatataaatgcgcctttgtggaggcgaattttgtatcctccttcgggctctaaagctttgctcaacgggatagaagttgactattttaagctttacaaattgccaaataaggatgttgaacaaggaaatgatatggtatga

SEQ ID NO: 143 - DNA, 1920 bp, Saccharomyces cerevisiae
atgagcgaatcagtgaaagaaaacgttactccgacacgtaacttccgtcgtactcaagggccccaaaataatacaaaacctcacaacgacaggaagaatttcagaagaaagcaaaagaaaaataacctttctgctgaacccaacctcaccacttcaagcgcagatgatactgatgaagaaaatgaattatgtgtaatttgtgcgcgcaagttaacatacgtatctctaacaccatgccatcataagacatgccatatctgtggtttcagacaacgagctttgtacaacaagaaaagctgtttgatttgcagaacggagaatgaagaagtcatgtttaccgatcgcatagatggtgatatttcagataaatataacttttgcgaaaaaaatgaaaaatatggaataaatttcactagcgaagaggttgctaccgaaacactaaatcttttaaaatttttttgtcccctatcgaaggatgaacaagtttgtgactttggtagtttcaagaaatataatgaacacttgaaatctgaacataacagaatgatctgtttaatatgtgcaacccataagcatgcattcccttgtgagcttgaaatattcacccaaaatcaattacgcaaccatcaaactaagggtaattcagaaggattcaaaggccaccctatgtgtgccttctgttccggaaaacgattttattcagacgacgaattgtacattcacatgagaaatcagcatgaaaagtgccatatttgtgataagatgaaccccgcttctcctcaatactttaaggattataatcagctttttgaccactttaaacattcgcattacgtttgcaccgtccaaacttgcttagataacaagtttgtcgtatttaaagacgagttggagttacaagcccacattttgcaggagcatggtaatatcctgaagggaaagccaaagtttttccagtcagaactttctacttttatctcggctccatctagagtgatcagggaaagagatgattacgaccttccatctatatcttccctaccgggttcatcatccggctctagaactgatgtgagatctgctagttctcccgaggaaagtagactaagattggcagaaagagcaaaatactatctggaaaattccaaagaagacttcaataagttttcttcttataacgaagattattctaaaggtcgcttaagtgctgaaaaattattggaatcatataaattgttattcacaaaaccaaatgcggatgtatatttattgattcataacttagctgaaacctttcccaaaaactcctccaaatacaataatctgaacgccatatatgaacaaagagagcaaacccttgcacgacaaacttcactaccttctttatctagtgattcttccctttcaatgtcaataggcaggggccattggggtggaacaaacgatgggggcagcgctggtgcagcattaggcgttaggaatatcaaaaatcttccaaccctgaaaagtccttcagcgtcgtatgatccctttgcaaccaccgtaaaaaaaaacactctgagacccgtgcaaaacattaaaagaactacacctcaatcagttagttatcgaacttccactaatacagtcgccttctctccaacctatttggaaagtaaaaagggttcctcttctacttctctaaataatagcaaagataaattgaaaagtctcaacctacctcaactacctccaccaaagccaaaggtgcaaattccaggtctgaacagaccccaaattgctgatcctaagcagtggggtaaaaagtcaagcacacaagatactaacgtgcacgataatttgagggaattgaacacaactagtggaggaaataaaaagaaaggcaaacaaaagcagctgttattccacattggtgtatag

SEQ ID NO: 144 - DNA, 441 bp, Saccharomyces cerevisiae
atgacaatcctccaccaatcagaagatcaaatagaggtggcttcagaggtcgcggcggcttcagaggcggcttcagaggtggcttcagaggcggtttctccagaggcggcttcggtggccccagaggtggatttggtggtccaagaggtggttacggtggctattccagaggtggctacggtggctactccagaggcggatatggtggctccagaggtggttacgatagtcctagaggtggttacgatagtccaagaggtggttattccagaggtggctatggtggtccaagaaatgattacggtcctccaagaggtagctacggtggttcaagaggtggttatgatggtccaagaggcgattatggtcctccaagagatgcatacagaaccagagatgctccacgtgaaagatcaccaaccaggtaagccatttatatag

SEQ ID NO: 145 - DNA, 393 bp, Saccharomyces cerevisiae
atgagcaggaaaacgttgccggaaaaagtttatttatccgagagaataattgatgaagaagtagcggtatgcacagtagcggcggaagtattagcaatttttactctggtgtgcacaagagtgttcatcatttttttcacggctaggatatgccatggaatatggccatcttcgccatcagagagaccgtaccacacgtttagagcagccaggttgcgaaactcttctaagatggtttccagcaattgtgtactatcagaatgtggacagtttaaaaggttgactgcgaatttgtcccaaaccgtatcaccgtcgcattttttgaatttgatcaaagcaccactcctaatagcacagcgatgctgtgagtgtgccagtgggaacgggtgctga

SEQ ID NO: 146 - DNA, 3036 bp, Saccharomyces cerevisiae
atggtaaaaaagaaaacgaataatgacaaaggaaaagaagtaaaggaaaatgaaggaaagctcgatatagatagcgaatcctcacctcacgaaagagaaaatgacaaaaaaaaaacagaagatgatagtctacgtgcaacagaaagtgaagagacgaatacacataatgctaacccaaatgaaacagtacgagctgataaattttcccaggaagagagtcgaccaatcgaagacagtccacatactgataaaaacacagcacaagaaagctgtcagcctagcagcgcagaagataatgtcataaatacagatattacaagcttaaatgaaaaaacctccacgaatgatgaacaggaaaaagggttgccactgaaaataagcgaaggtccttttacaatctctaccctattagacaatgtaccatctgacttgatatacacatgttgtgaggcgtatgagaaccacatattcttgggaacaactacaggtgatcttttacattactttgaacttgaacgtggaaactatatgcttgtgtcgcaaacaaagtttgatgcagaatcaaattcgaagatagacaaaatccttcttttacctaaagtagaaggcgcactaatattatgtgataacgaactagttttatttattctcccagaatttgcaccaaggcccaacacaactagacttaaaggtattagtgatgtcgttatttgtaatttttcccgaagctcgaaggcttatagaatatatgcatttcacgcagaaggcgtaagactactcaaaatatctgcagattcattagtacttaccaaagcttttaatttcaaactaatagataaagcttgtgcgcacgaagaaacattaatggtttcgaagttaaatagttacgaacttataaatctaaagtcgtcacaggtaattccattatttcgaataagtgagactgacgaagacttagaacctataataaccagtttcaatgaacagagtgaatttttagtttgttctggaggaggatcatacgatagtggggcgatggccttagtggtaaatcatcatggcgacataataaaaggaactatagtgctaaaaaattatccaagaaatgtaattgttgagtttccctacataatcgctgagtctgctttccagtcggttgacatatattctgccttgccgagtgaaaaatcccagttgttgcaaagtataactacttctggatcggatttaaaaatttcaaaatctgacaatgtattcactaataccaataattctgaagaattcaaagaaaaaatattcaataagttgagacttgagcctctgacacatagtgataataaatttagaattgaaagagaacgcgcctttgttgaagagtcttatgaagaaaagacctctttaatcgtttataataatttagggattcatttattagttccaacgccgatggtcttacgttttacatcatgtgaagagtccgaaattgataatattgaagatcaattgaaaaagcttgcgaagaaggatttaacaaaatttgagcacattgaagcgaaatatttgatgtctcttttattgtttctaatgaccttacattacgatcacatagaagatgaggtaatgaaaaagtggtgtgacttttctgacaaagttgacatcagaatattattttatatgtttggctggaaagtctatagcgagatctggtgcttccatgggttaataaatatagttgaaaggttgaaaagcttgaagctaactaacaaatgcgagaatattctaaaaatgcttctgatgatgaagaatgaactaaagaaaaagaataaaactggactcttaacgaatgactttgatgatataatgaaaactattgatataactttattcaaattaagattggaaaaaaaggaaactataactgttgatatgtttgaacgcgaaagttacgacgaaattatcagagagattaatttacatgatgataaacttcctaggatagagttactaattgagatatataaagaaaaaggggaatatcttaaagcactaaatcttttgagggaagctggggattacatttcactagtttcattcattgaagaaaatctcaaaaaacttcctgaggattatatcaaagaaagaatagccgatgatcttctcctcactcttaaacaaggcgacgaaaatactgaggaatgcgccatcaaaaaggttcttaaaatattggacatggcatgtattaataaaaacgactttttaaacaaaatacccgcagaagaaacatccctgaaggtatcattcatagaacaactcggtgtccaaaacagcaacgattccaaatttctctttaactattatttggcgaaattgcgagaaataatcaaccaaagtaacatatggtccattcttggagattttatcaaagagtacaaagacgatttcgcgtatgataagacagatataacaaattttattcatatcaaattaaagcatagtcttcagtgcgaaaatttttccaaatattatgaaaaatgtgaaaacttgaaatcggaaaatgaaaaagatgatgagtttattaatttcaccttcgacgaaatttccaagatcgataaagaacacatattgaccttgttatttttccccaatgaattaacaaactgggtttcttccgaagaattgctgaaaatatatttgtcatttaatgatttcagaagtgtagaaaaatacataggcaagcagaacttggttgcggtgatgaaacaatacttggacatatcttctttgaattactcagttgaattagtgactaatttgctacagagaaattttgaacttttagatgatacagatattcaattaaagatcctcgaaactataccttccgtatttcccgtacaaacaatatccgaattgcttttaaaggtacttattaagtatcaagaaaagaaggaggaatctaatttgcggaaatgtttgttgaaaaatcaaatatccatttctgacgaactaagccggaattttgatagtcaaggataa

SEQ ID NO: 147 - DNA, 1158 bp, Saccharomyces cerevisiae
atggatacagggagtgcgagcataaaggactatgagaccgtgttaacggatattgaggattctattgctgtgagcagcgaagaagtgctaaataaccaagaactaaggctcaaaaatacattgcacgaaattacttccagcattctggcaataaatgaagagaacaagtttgtgaatcctcttaggaacgatgagagcttagacgtagagggaaaggaggtctttgtaaatccaaaaattctgagtgcaaaaatcaaggagttcaataaactaatggagttacttaagttgacttacttggaacaggagacattggactatttcttcaggtttactttgtcctcgacgaaacctttgcagttagattccgaaaaagacccccaatttgtaaaacttaatgaaagagttaatgatttaaaggaagaaatttcgaatgttcaagaaagcaaaatagaacaaatcaaagctgaaatccaagagaccggtcacaactttgcagaaaggcaagatttaataaatgagttgtatctggaagccactggtgatattgaaaattgttgggattcgctcaatgaactaaaaaatttaacaaacaaagaagataagaatatgatgggagaaaaagatacgatattaaattcaagtgattcagatgattttgtggaagagacatacacgaattggcaaaaactactatttttgcagaagcaaaatcaaaggttaacgaaagagctcaaggaaatgcatgaagttaaaaatcaaataatacgaaaaggagagcaatcaaaaaaagaggattctggacatttaatggccaatgaatcagaattatgtcagtcaattaatttgctaacaaaattttgggaaaagcactttttactcaaagggtcaaagacgactatattaaattttgaaatctttacccagcttggaaaagtacaatttgaaataaaggatatgcaatatataattgccatatctttaagcgatttaaaaaggccaatgataaaagacattactattttacaaaaagcgggtggcaatatagtcacagacattgaagcgagcagtaaatttaacaataagtacagaaataatactaaggtgcaaatctttgaggtaatggatgacattataagcgagctaacaaacgaataa

SEQ ID NO: 148 - DNA, 501 bp, Saccharomyces cerevisiae
atgagcggtagtaggggcaatagcagcaatagtagcgtcagtaataatagtaataataacaataataatgacgggggcgacgagagattgctgtttctaagaagcgtgggcgaacgcaatgaaattggctttccctctagattcaagtcggcgcattacaagaaaccgacaagaagacacaaatcagcgaggcagttgatctcggacgaaaacaagcggatcaacgccttgttgaccaaggctaacaaagctgcagagagttctactgctgctaggcgacttgtgcccaaagcgacgtactttagcgtggaagcgccaccgtctatcaggcctgccaagaagtactgcgatgttactgggttgaagggcttctacaagtcgcctacgaacaacattcggtatcacaacgcagaaatctatcagttaatcgtaaagccgatggcccccggcgtcgatcaggagtacttaaaattgagaggggccaacttcgttctaaaatag

SEQ ID NO: 149 - DNA, 2667 bp, Saccharomyces cerevisiae
atgtacgtttataaaagagacggtcgtaaagaacctgtccaattcgataagattaccgctcgtatatcacgcttatgctatggtttagatccaaaacatatcgacgccgttaaggtcacccaacgtatcatttctggtgtctatgaaggtgtcacaacaatcgaactagacaacttagccgctgaaacatgcgcttatatgactactgttcatccagattacgccaccctagcggccagaattgccatttctaatttacataaacaaaccacaaaacaattttctaaggttgtcgaagatctttatagatacgtcaatgctgctactggtaagcccgctcccatgatctctgatgatgtctacaatattgtcatggaaaacaaggataaattgaactccgcaattgtctatgacagagattttcagtacagttattttggttttaaaactttggaacgttcttatttactaagaatcaacggtcaagtggccgaacgtccacaacatttaattatgagagtcgcactaggcatccacggtagagatatcgaggctgctttagaaacgtataacttgatgtctctaaaatattttactcacgcctctccaacgttgttcaatgccggtactccaaaacctcaaatgtcctcttgtttcttggttgccatgaaggaggactctatcgaggggatttacgacaccttgaaggaatgtgctttgatttccaaaactgctggtggtattggtctacatatccataacattcgttcaactggttcttacattgctggtacaaacggtacttctaacggtttaattcctatgattcgtgttttcaataacactgcccgttatgttgaccagggtggtaataaaagacctggtgcgtttgccctttacctggaaccatggcatgctgatatatttgattttattgatattaggaagaaccacggtaaagaggaaattcgtgcaagagatttgttccctgctctatggattcctgatcttttcatgaagcgtgtcgaagaaaatgggacctggacattattctctccaacatcagctcctggtttaagcgattgttacggtgacgagtttgaggctctatatacccgctacgagaaagaaggtcgtggtaagactatcaaagcccaaaaattatggtattccattttggaagctcaaactgaaactggtacacctttcgttgtttacaaggatgcttgtaacagaaaatctaatcaaaaaaacttaggtgtcatcaagtcatcaaacttatgctgtgaaattgttgaatactcagctccagatgaaactgctgtttgtaacttggcttccgttgccttaccagcattcattgaaacttctgaggatggtaagacttccacatacaacttcaaaaaattacatgaaattgctaaagttgttactcgtaatttaaacagagtcattgatcgtaattactaccctgttgaagaagcgagaaaatccaatatgagacatagaccaattgctttgggtgttcaaggtctcgctgacactttcatgctgttacgtttgccatttgattctgaggaagcccgtttgctaaatatccaaatctttgaaactatttatcatgcctccatggaagcttcttgtgaactagctcagaaggacggtccatacgaaactttccaaggatctcctgcttctcaaggtatactacagtttgatatgtgggaccaaaaaccctacggcatgtgggattgggacaccttaagaaaagatatcatgaagcatggtgttagaaattccttgaccatggcaccaatgcctactgcatccacatcccaaatattgggttataatgaatgtttcgaaccagtcacttccaatatgtactcccgtcgtgtcttatccggtgaattccaggttgtgaacccttacttactgcgtgacttggttgatttaggtatttgggatgagggtatgaaacagtatctgattacacaaaatggctccattcaaggcttaccaaacgttccacaagaattgaaggacttatacaagactgtttgggaaatttcacaaaagactatcattaacatggcagccgatcgttctgtctatattgatcaatctcattctttgaatttgttcttacgtgccccaactatgggtaaactaacaagtatgcatttttacggatggaagaagggattgaagaccggtatgtactatttgagaacccaagctgcatctgctgcaattcaatttactattgatcagaagattgcggatcaagctacagaaaacgttgctgatatttccaacttgaagcgtccatcatatatgccttccagtgcaagctacgctgccagcgatttcgtgcccgcagctgtgactgcaaacgcaactattccatctctagatagctcctcggaagcttcaagagaggcatctccagctccaacaggtagccactcattaactaaaggaatggcagaattaaacgttcaagagtctaaggtagaagttcctgaagtacctgccccaactaagaatgaagaaaaagctgcccccatcgttgatgatgaggaaaccgagttcgacatttacaactctaaggttatagcatgtgctattgataacccagaagcttgtgaaatgtgttcgggttaa

SEQ ID NO: 150 - DNA, 1731 bp, Saccharomyces cerevisiae; identical to SEQ ID NO: 89

SEQ ID NO: 151 - DNA, 2739 bp, Saccharomyces cerevisiae
atgaacatgcaagacgctaacaaagaagagtcgtactcgatgtacccgaaaacctcttctccaccaccacctacgccaaccaatatgcagattcctatttatcaagcgcctttgcagatgtacggctacactcaggccccatatctataccccacacaaatacctgcctattcgtttaatatggtcaaccaaaaccagccaatctaccatcaaagtggcagcccacatcacttgcctccgcaaaacaatattaacggcggaagcactaccaataacaacaacattaacaagaagaagtggcactctaatggcattaccaataacaatggaagcagcggtaatcaaggcgccaactctagcggtagcggcatgagctacaacaaatcccacacctaccatcacaattactctaacaatcatatccccatgatggcctctccaaacagtggcagcaatgcgggcatgaaaaaacagaccaactcttccaacggcaacggttcttcggctacttcaccatcgtactcttcctacaactcttcttcacagtatgatttatacaagtttgatgtcactaaattaaagaatctcaaggaaaattcatcaaacttgattcaattgccactgttcataaacactacggaagcagaatttgctgcggcaagtgtccaaaggtacgaattaaacatgaaggctttgaacctaaactctgaaagcttagagaactcatctgtagaaaagagctctgcccatcatcacacaaaaagccatagtataccaaagcataatgaggaagtaaagacagaaacacatggggaagaagaagatgctcatgataaaaaaccacatgcgagcaaagatgcgcacgagcttaaaaagaaaactgaagtaaagaaagaggatgctaagcaagaccgtaacgaaaaagttatacaggaacctcaagctactgttttacctgtagtggataagaaggaaccagaggaatctgttgaagaaaatacttccaagacatcttcaccttcaccatctcctccagcagcaaaatcctggtccgccatagcatcagatgcgattaaaagtagacaagctagtaacaaaacagtctccggatcgatggtcactaaaacaccaatttctggtacgaccgcaggcgtttcatcaacaaacatggctgcggcgactataggtaaatccagctctcccctgttgtccaagcagcctcagaaaaaggataaaaaatacgttccaccttctacaaagggtattgagccactgggttcgattgcgttaagaatgtgttttgatcccgatttcattagttacgttttacggaataaagatgttgaaaacaaaataccagtccattccattattccaagaggcataattaacagagccaacatttgttttatgagttctgtgttacaagtgttactctactgtaagccatttattgatgtaattaacgttctcagtacacggaataccaattcaagagtcggcacatcatcctgtaaattattagatgcttgtttgactatgtataagcaattcgataaggaaacctatgagaaaaaattcctagagaatgctgatgatgctgaaaaaaccacggaaagtgatgcaaaaaaatcatcaaaatccaagagtttccaacactgcgccactgccgatgctgtcaaacctgacgaattttacaaaactttgtctactataccgaagttcaaagacttgcaatggggccatcaggaagacgcagaagaatttttgacccacttattggaccaattacacgaggaattaatttctgcaattgatggcttaaccgataatgaaattcaaaatatgctgcaaagtattaatgatgaacaattgaaagttttctttattagaaatttgtcacgttatggaaaagcagagtttatcaaaaatgctagtcctagactgaaggagttgatagaaaaatatggcgtgatcaatgatgactctaccgaagaaaatggttggcatgaagtgagcggatctagcaaaagaggcaagaaaactaagaccgctgccaagaggactgtcgagattgttccatcaccaatctccaaacttttcggtggccagttcagatctgtgttagatataccgaacaataaggaatctcaatcgattacactcgatccgttccaaacaattcaattggacatttcagatgctggtgtgaatgatctagaaactgcattcaaaaaatttagtgaatacgaattgctaccctttaagtcctcgtcagggaatgatgtcgaggccaagaagcagacttttattgataaattgccgcaagttcttttaatccaattcaaaagattctcattcataaataatgtgaacaaagacaacgcaatgacgaactataacgcgtacaatggacgtattgagaagatcaggaaaaaaattaaatatggtcacgagttaatcatacctgaagaatcaatgtcttccataacattgaaaaacaacacctcagggattgatgatagaagatataagctaaccggagttatataccatcatggggtaagttccgatggcggtcattacacagcggatgtttatcatagcgagcacaacaaatggtatagaatagatgatgtaaatattaccgaactagaggacgatgacgttttgaaaggtggcgaagaagcttctgattcgaggactgcctatattttaatgtatcaaaagagaaattaa

SEQ ID NO: 152 - DNA, 1500 bp, Saccharomyces cerevisiae
atgagcggagaaacgtttgagttcaatattagacattctggtaaagtttacccaataacactttccactgatgctacttcagcagatttgaaaagcaaagcagaggaattgacccaagtcccaagtgcccgccaaaaatacatggttaaaggtggcttgtctggcgaagagtccattaaaatatatcccttaatcaagccaggatcgacagtaatgctattggggactccagatgctaacctgatttctaaaccagccaaaaagaataatttcattgaagaccttgcgcctgagcaacaagtccaacaatttgctcaattgcctgttggtttcaagaatatgggcaacacctgttatctgaatgctaccctacaggctttatacagagtgaacgatttaagggatatgattcttaattataacccttctcaaggtgtgtctaacagtggtgcacaagatgaagagattcacaaacaaatcgttattgaaatgaagcgttgttttgaaaatttacagaataaaagtttcaagagtgttttgccaattgtgttattaaacacgctaagaaagtgttatccacaatttgctgaacgtgattcacaaggtgggttctataaacagcaagacgctgaggagttgtttacacaactattccatagtatgagtattgtttttggtgacaaattttccgaagatttcaggattcaatttaaaactaccatcaaagacacagctaatgataacgatattactgttaaagaaaatgaaagcgattctaaattacaatgtcatatttctggtactacaaatttcatgagaaatgggctcctggaaggtttgaatgagaaaattgaaaaaagatcagacttgactggcgccaattccatctatagcgtcgaaaagaaaatatcaagattaccaaagtttttaactgttcagtacgttagatttttctggaaaaggtcaaccaacaaaaaatctaaaatattgcgtaaggtcgttttcccatttcaattagatgttgcagacatgcttaccccagaatacgcagcagagaaggtaaaagttcgtgacgaactgagaaaagttgaaaaggagaaaaatgaaaaggaaagagagatcaaaaggcgtaaatttgacccatcatccagtgaaaatgtcatgacaccaagagaacaatatgagacacaagtggctcttaacgaaagtgaaaaagatcaatggctcgaagagtataagaaacattttcctccaaacttggaaaaaggtgaaaacccatcttgtgtttataacttgatcggtgtcattacacatcaaggtgccaattctgagtctggacactatcaagctttcataagggacgaactggacgaaaataaatggtacaaatttaatgatgataaagttagcgttgttgaaaaggaaaaaattgaatctttagccggtgggggcgaaagtgatagtgcactgatcttaatgtataaaggatttggtctgtaa

SEQ ID NO: 153 - DNA, 1422 bp, Saccharomyces cerevisiae
atggcaaaggataatagtgagaagctgcaggtgcagggagaggagaaaaagtccaagcaaccggttaatttcctgcctcagggtaaatggctgaagccaaatgaaatcgaatatgagtttggtgggactactggtgttattggtatgctgatcgggtttccactgctaatgtactatatgtggatttgtgcggaattttatcacggtaaggttgccctacccaaggctggtgaatcgtggatgcactttatcaagcacctataccagttagtcttggagaacggtatcccagaaaagtatgactggactattttcttaacattttgggtgtttcagatcattttctactatacgttgcccgggatttggacaaaaggtcaaccattgtctcatttgaagggaaaacaattgccttacttttgtaatgccatgtggaccttgtatgtaactaccactttggtcttggttttgcactttaccaatctttttagattgtatgtcattattgaccgttttgggaggatcatgacatgtgccattatttcagggtttgccttctccatcatattgtacttatggactttatttatctcacatgactatcatagaatgacaggaaaccatctatatgatttcttcatgggagctccactaaaccctaggtgggggattttggacttgaagatgtttttcgaggttagattaccttggttcaccctttactttatcactttgggtgcctgtttgaagcagtgggagacttacggctatgtgacaccacaattgggggttgtcatgttagctcattggttgtacgcgaacgcatgtgctaaaggtgaagaattgattgttccaacctgggacatggcttacgaaaagtttggatttatgctgatcttctggaatattgccggtgtcccatacacttactgtcattgtacgttgtatttgtactaccatgacccatctgaatatcactggtctacactgtacaatgtttcgctgtacgttgttctattatgcgcctactacttctttgacacggcaaatgctcagaaaaatgccttcagaaagcaaatgtctggtgacaagacaggtaggaagactttcccatttttgccataccaaattttgaagaatccaaagtatatggttacctccaatggatcgtacctattgattgatggttggtacactttggctagaaaaattcactacactgccgattggactcaatctctcgtttgggccttgtcttgcgggttcaactcggtgttcccatggtttttcccagtattcttccttgttgtcctgattcacagagccttcagagaccaagcaaaatgtaagagaaagtacggaaaagattgggatgagtattgtaaacattgcccttacgtctttattccttatgttttctag

SEQ ID NO: 154 - DNA, 1443 bp, Saccharomyces cerevisiae
atgtctaggaagttgtcccacctgatcgctacaaggaaatcaaaaacaatagtcgtaaccgtacttcttatttattctttgttgacatttcacttgtcaaacaaaaggctgctttctcagttttaccctagcaaagatgatttcaagcaaactcttctccctacgacttctcattcacaagatataaatttgaagaaacaaattacagttaacaagaaaaaaaatcaattgcataatttacgtgatcaattatcgtttgcgtttccctacgactctcaggcccccatcccgcaaagggtgtggcagacctggaaagtcggcgcagatgataagaattttccctcttcgttcagaacctatcaaaaaacatggtctggttcgtattcaccggattaccaatattctctgatttcggatgattctattatcccttttttggagaatctttacgcacccgttccgatagtcatccaagcgtttaaattgatgcctggaaatatcctaaaggcagattttttaaggtacctattattatttgcaagaggtggtatttactcagatatggatactatgcttttgaagccaattgattcatggccttctcagaataagtcatggctaaacaacataatagatttgaataaacctattccttataagaactcaaagccctcacttctctcaagtgatgagatatcacaccagccaggtttggtcatcggcattgaggcagatccggacagagatgattggagtgaatggtatgctcgtaggatccagttttgtcaatggactatccaagccaaaccaggtcacccaattctaagagaattgatcttaaatattactgcaacgactttggcgagcgtacaaaacccaggagttcctgtcagtgaaatgattgatccaagatttgaagaagactacaacgtaaactataggcacaaaagacgtcatgatgagacctacaaacactccgaattgaaaaataacaaaaatgttgatggctcagatataatgaattggacgggtccaggtattttctccgacattattttcgaatacatgaacaatgtgctccgatacaatagcgatatcttgttaatcaacccaaacctaaacaagaacgacgaagaaggtagtgagagtgccaccacaccagcaaaagatgttgataacgatacgctgtccaaatcaacaagaaaattttataagaaaatatcagagtctttgcaatcgtcaaattcaatgccctgggaattcttctcttttttgaaagaaccagttatcgttgatgatgtgatggttctaccaataacaagtttttcaccagatgtggggcagatgggcgcccagtctagtgacgacaaaatggcttttgtaaagcacatgtttagcgggagctggaaagaagatgctgataaaaatgcaggtcataaataa

SEQ ID NO: 155 - DNA, 1188 bp, Saccharomyces cerevisiae
atgtcactttctcttgtatcgtaccgcctaagaaagaacccgtgggttaacatttttctacctgttttggccatatttctaatatatataatttttttccagagagatcaatctctgttgggacttaatggccagtccatttcccaacacaaatgggcacacgaaaaggaaaacacattttattttcccttcaccaagaaatacaaaatgccaaagtactcttataagaaaaaaagcggctggttgttcaacgatcacgtggaagatattatcccagaaggtcatattgcacattatgatttgaacaaattgcactctacgtcagaagcagctgtcaataaggagcatattttgatattgactccaatgcaaacatttcatcaacaatactgggacaatttgctgcagctaaattaccctcgggaattgattgaattggggttcattacaccaagaacagccactggtgacttggccttaaagaaattggagaatgctattaaaaaggttcaaacggacaagaaaactcaaagatttagtaaaattactattttgcgacagaattcccagagttttgataagttgatggagaaggaaagacacgctttagatgttcaaaaggaaagacgtgcagcaatggctttggcgcgcaatgaattactattctccaccataggacctcacacttcttgggtgctgtggctagatgccgatattatagagacaccaccatctttaattcaagacatgaccaaacacaacaaagctatcttagctgcaaacatttatcaaagattttacgatgaagagaagaagcaaccatcaatcagaccatacgatttcaacaactggcaagaaagtgacaccggtttagaaatagcctctcagatgggtgatgacgagattattgtcgagggttatgcagaaattgccacttataggccattaatggctcatttctacgatgctaatggcgtaccaggtgaagagatggcgctggatggtgttggtggaggctgtactttggtcaaagcagaagttcacagagacggtgccatgttccctaatttcccattttatcacttgattgaaacagaaggttttgctaagatggcgaagagattaaactacgatgtatttggcttaccaaactatttggtttatcacatagaggaagagaaccattga

SEQ ID NO: 156 - DNA, 2535 bp, Saccharomyces cerevisiae
atgaaaccttacttatttgacctaaagctaaaagacacagagaagctggattggaaaaaaggcctctcctcatatctcaagaaatcatacggctcctcacaatggagaacattttatgatgagaaagctacttcagaactggatcacttaaggaataatgcaaatggagaactagcaccttcttcattgtcggaacaaaatctaaaatattactcatttttggagcatctttattttcgcctgggtagcaaaggatcaagattaaaaatggatttcacttggtatgacgcagaatactcatcggcccagaaaggattgaaatacacgcaacatacgttagcatttgaaaagtcttgtactttgttcaacattgctgtaatctttacccaaattgcgagggagaatatcaatgaggactacaaaaactcaatcgcaaatttgacaaaagctttttcctgttttgaatatctatcagaaaattttttgaactcaccttcagtcgatcttcagtcagaaaacactaggtttctggctaatatttgccatgcagaagctcaagaattgtttgtcttgaaattattaaatgatcaaatatcatccaagcaatatacattaatcagtaaactttctagagccacgtgtaacctctttcagaaatgtcacgattttatgaaagaaatagatgacgatgtggccatttatggtgaacccaaatggaaaacgacagttacttgcaaactgcatttctacaaatcgttaagcgcttattatcacggtttacaccttgaagaagaaaatagagttggcgaagcaattgcttttctcgatttttctatgcaacaattgatttcatcccttccattcaaaacgtggttagtggaatttatagactttgatgggtttaaagaaactttagaaaagaaacaaaaggagttgattaaagataacgattttatatatcatgaaagcgttccagccgttgtgcaggttgattccattaaggcgctcgatgcaataaaatctccaacatgggagaagatattagaaccatatatgcaagatgttgcaaataaatgtgactctttgtacagaggaattattcccctagatgtctatgaaaaggaaagtatttactcagaagaaaaagcgacgctgttgagaaagcaagttgaagaaactgagacagcaaatttggaatattcttccttcatcgaatttacaaatctacccaggctcttgagtgatttggaaaaacaatttagtgacggaaatattttctcgaatacggatacacagggacaattgatgagggaccaaattcagacatggtgtaaatttatccaaacaaatgaatttagggatatagaagaacagatgaacaaaattgttttcaaaaggaaacaaattttagaaatcctttctgccttacccaatgatcaaaaagaaaatgttacaaaactaaaatcttctttagtagctgcttcaaactcagacgaaaaattgttcgcatgcgtaaaaccacatattgtcgagatcaatctattgaatgacaatggaaaaatatggaagaagtttgacgaatttaatcgcaatacgcctccacaacctagcctattggatatcgatgataccaaaaacgacaagatattagagttgttaaaacaagtaaagggccatgcggaagacttaagaacattgaaagaggaacgttcgagaaatttgtctgaactaagagacgaaatcaacaacgatgatatcacaaaattattaattattaataaggggaaatccgatgttgagctcaaagatttattcgaggtggaactggagaaattcgagcctttgagcacaagaatagaggcgacaatttacaaacaatcttcaatgatagatgacatcaaagccaagctagatgaaatttttcacctttcaaatttcaaggataaatcttctggggaagaaaaatttttagaagatcgtaagaatttttttgataagctgcaagaagcagtgaaatcattcagtatttttgcatccgacttgccaaaaggaatagagttctatgattcattattcaatatgagtagagacttagcagaaagagtgagagttgcaaagcagaccgaggattcaacagctaattctcccgctcctcccctccctccacttgattctaaagcgtctgtcgttgggggtcctccattactgccccaaaaaagtgcagcctttcagtcattatctagacaagggctcaatttaggggaccaatttcaaaatctcaaaataagtgccggcagtgatttacctcaaggacccggtattccaccaagaacttatgaagcttcgccatatgctgcaacgcctactatggcagccccaccagtaccaccgaaacaatcgcaagaggatatgtacgacttgagaagacgtaaagcagttgaaaacgaagaacgtgaactgcaagagaatcctacgtccttttacaatagaccctctgtttttgatgaaaatatgtactccaaatacagcagttag

SEQ ID NO: 157 - DNA, 735 bp, Saccharomyces cerevisiae
atggtagctacagtaaaaagaaccatccgtataaaaacccaacaacacattctgccagaagtgccaccagtagagaattttcctgttcgtcaatggagtatagaaattgtattattggatgatgaaggaaaagaaattcccgctacaatttttgacaaagttatttaccacttgcacccaactttcgccaatcctaacaggacatttactgacccaccatttagaattgaggaacaaggttggggtggatttcctttagatataagcgttttcttgttggaaaaagcaggtgagcggaaaataccacacgatttgaactttctacaagagagttatgaagtggagcacgttatccagattcctctcaataaaccacttttaacagaagaacttgcgaaaagtggatctactgaagaaacgacggccaatacaggcaccattgggaaaagaaggactaccacgaacacaactgcagaaccaaaggcgaagagagctaagacgggcagtgcatctaccgtgaaagggagcgtggacctagaaaaattagcgttcggattgactaaactaaatgaagatgacctggttggtgttgttcaaatggttaccgacaataaaacaccagaaatgaacgtgacgaataatgttgaagagggtgaatttataattgacttgtatagtttacctgagggattattgaaaagtctatgggactacgttaagaaaaataccgagtaa

SEQ ID NO: 158 - DNA, 1467 bp, Saccharomyces cerevisiae
atgtcagctatacaatcgccagcacctaaacctctgcagccaacttatccagctgcttcgccagcttccacgaatgcatatatgaagccgggcctaattggtagtcctgccgtcagtaatcataccgagcctaacaatggtaacaatgaaactgcggagcctcagggaccaaatcagagaattgatttgggtgccatgatcgaagaattaacctcactattgggaaaagaaagctggacgaaatatgctcagatcatcagtcttttcattttagggaagttatccagaaaggaactttctaatgaattagagctggtattttcaccaagcgctgcaagcttagaaaaatcaaatacaaatcatcaccatagtttagtacgacttcataaccaacttttattagggatttttgctaattcattacgtgaaaaccctcttgggagaaacggtaatgagagttcttggggatttggcaatggaagcaacaatccaaacaataaactaaaaagaatcaataagcataactctcaaattgaagtctataaaaaaattgtcatgtcgttacctctaaatgatcgaaatagacttaaaatgatcacgaaagaggccggcaaaagaggcttcattttttgctctgtatttcaagccagattaaataatatacccaaaattcccattgtaaccaatccagaaagtttgaagcgtgtcaagagcaataatttaaaaacgccgctggaatggtcacaagatataatgaatggattcaacgttcctttagcgagcgaaagccattctttaccagacacggattcgttttacttaagaatggttggtatagcaagagaacatgggttagtcggcacagtggacgcacgttgcgtagagcttatatcattggccctagatcaatatctaaaaaatataatagagtttactattgatacagttcgttatagaaggaagaaatattcagattattatgatttgaatgagagtgggctttataaatccgtatcagaaatggctgctgataaacgcgatgccaaaattaagcagttagatgatgataaaaatgaggatgaatgtgccgatgaagccaaaagtattaataacggcaataacagtagcaaggacgatattggcgacatatcaatgagcagcattacaaaggctggcgaagcagttaatgaggagttacatgaaaacagaacgatatcattaacgaatgaagacatatatgattcgttatctatatttccgaatttggttgaaccttcaggttcatactatgcgttaactaatttagggctagttaacgatgatgaactagtagatatgaagagcaatattgacgacttgccggacttcttaaatgaaaagccgactttcacgcctttggatgaaagaaacgttggtacaaggcatgaattaaattggttaattaaaggaatcttaacggaagattga

SEQ ID NO: 159 - DNA, 2610 bp, Saccharomyces cerevisiae
atgactgaaagtgctatagatgaccaaaggtttaatcttacaaaggagttacaacgacactcatgcagagatcaagggaaaataacacaaaaggatgatgctttagattttataagttacagttcttttcaatcctcttttaatactgaccaaaaaagcgcgaacaatggaagtactgtgagaagaagcataaggtcgatttttagaagagcagccgaattgcctagagtccatatggggcctcttacttattcacatgggataaatgagcttgttaacaagaaattaagaaaagactgtgatctcagcacgctatgtcgcgtattgcaaagaggaatcaggatgattaggatgacaagaagaagaaggaagttctatgaatttaaattaatcaataacaacgggcaaataatatggaaagatggttcgaagtatctggaattagactccgttaaagacatcagaatcggtgatacggcgagtacttatcaagaggaagtagatccgaaaagattgagatcggacagtaaactttggattgctattatttataaggtttccaacaaattgaaagcgttacacgtagttgctttgaatgaattggactttaacacattcttaagctgcatttgcggtttagtgaagttaaggagggaattaatggaaagcatacttttgcctgataattcgcaattcgctaggatacattggcaaattactgtttctgaaaaagaggaagatgagaaaaaggacacattgagctttgcagatgtgaagaaactatgtgataaatttcatatttatgtgtcaactggtcaattgttagagtttttccaattagcagacatcaatcataacggactattaaattattttgaatttgaaaagttcattaaaatcttaaagaataggaaagaggtaaatatgatttggtctaaatttacaaaacctccacactcccacttatcatttgaaaatttttttcaatttttaattacagagcagcatgagcaagtggatcgtcaaactgcatggtcatattttattaagtacagagaaccgactcagctgacgatggggcaagatggttttaccaagttcttgaaggagcaaccatatttggtggaggttaaagaggagttatattcaaagccattaaaccattattttattgcatcttcacataatacttatttattggggaaacaaattgcagaaactccatctgtggagggatatattcaagtcctacaacaaggttgtaggtgcgtagaaattgatatatgggatggtgaaaatgggccagtggtatgccatggatttttaacgtcagctattcccttaaaaacagttatacgcgtgataaagaagtacgcttttataacgtctccgtatcctttgattatttcactggaaataaactgtaacaaggataatcaaaaattggcaagcctgataatgagagaggttttagcagaacagctgtactttgttggcacaagaactgataaactgccatcaccaagggaattaaaacataaaatactgttgaaatccaagaagacgagtgaagcaactagaggtcttagcgttaatgagccatttccatcttcttttagttcttcttacgaatcagctaatgaacaggaattaagaatgaaagatgactccacgaacagtagtagcgcaacaaattcttctagtatgcaacgcatcaaaagaattggattaaaaaagcacgctgatataattaatgatgtttctaacatttctggaatacatggcatcaagttcaggaatttctctttaccagagtccaaaacgatagcgcattgtttttcactgaatgagcgcaaagtagaatacatgataaaagacaaacacttaaaattatcgttggataaacataatagaagatatttgatgagagtctatcctcacgttttacgatacaaatcatcaaattttaacccaatacctttttggaaggccggtgtacaaatggtggctacgaattggcaaactaatgatattggacagcaacttaatctggcaatgtttcaaatattagatcaccaacctgatggaagttttaaatcaggatacgtgctaaaaccaaaaaagttgctacctgtagtaacaaaggcgaaaatgattccattaatttacgaacattttgaaaatggtagtgatcctgttactgttaaaattcgaattttatctacacagttattgcccagattgaatgatacatcgccaagcagaaataacaccaattcctttgttaaagtggagtttcataccgatgacgagccgacaatgcccatctcaatcgataagggtacaaggatttctgccactgaagcatccactaagagttctcaagggaatggattcaacccaatatgggatgctgaagtttctatcacattgaaagatacagacctcacatttataaaattcatggttatctctgaagagacccagattgcatctgtttgccttaagctgaactatttgaggatgggatatcgtcacattccgttgtttaacatggagggggaacaatacatattttgcactttatttattcatacacaaattttatag

SEQ ID NO: 160 - DNA, 519 bp, Saccharomyces cerevisiae
atgtccacaccagctagaagaaggttgatgagagattttaaacgtatgaaggaagatgccccaccgggtgtatctgcttcaccattacctgataacgtcatggtatggaacgccatgattatcgggccagccgatactccatatgaagacggaacttttaggttattgttggagtttgatgaagaatatcccaataagccaccgcatgtcaaatttttgagtgaaatgtttcatcccaatgtctatgcaaatggtgaaatttgtttggatattttgcagaacagatggactccaacatatgatgtcgcatccatattgacatccattcaaagtttattcaacgatccaaatccagcttcgccagcaaacgttgaagctgcaacattattcaaagatcataaatcacagtacgtcaaaagagttaaggagacggtagagaaatcttgggaggatgatatggacgatatggacgatgatgatgatgatgatgacgacgacgacgacgacgaagcagactga

SEQ ID NO: 161 - DNA, 369 bp, Saccharomyces cerevisiae
atgactacgtttagattttgtcgtgactgcaacaatatgttgtaccctcgtgaggataaagaaaataaccggttgttatttgaatgcagaacatgctcgtatgtagaagaagcgggcagtccattggtctacaggcatgaattgattaccaacattggtgaaactgcgggagtggtgcaagatattggttccgatcccactcttccgagatctgatagagaatgtcccaaatgtcactctcgggaaaatgtgttttttcaatcacaacaaagaagaaaggatacttcgatggttttattctttgtgtgcttatcttgctcacacatatttacttcagatcaaaaaaacaaaaggacgcagttttcatga

SEQ ID NO: 162 - DNA, 756 bp, Saccharomyces cerevisiae
atggacctagatctagccagtatcttaaaaggtgaaatttctaagaagaagaaagagctggccaactctaaaggtgttcagccaccatgcactgagaaattccagccacatgaatccgcaaacatagacgaaacaccgcgacaggtagaacaagaaagtacagatgaagaaaacctgtcagacaatcagagtgacgatattaggaccaccattagcaaattagaaaatcggccagaaaggatacaagaagcgatagctcaagacaaaaccatctctgtaatcattgatccatcacaaattggttctactgaaggaaaacctcttttgtcaatgaaatgtaatttatatattcacgaaatattatctcgttggaaggcgtctttggaagcttatcatccggagctgttcttagacactaaaaaagcactctttccactcctactgcaactacgaaggaaccagcttgccccggatttacttatttcccttgcgaccgttctctatcatttacaacaacctaaagaaataaatttagccgttcagtcctatatgaaactcagtatcggcaatgtggcctggcctattggtgttactagcgtaggcattcatgctcgtagtgcacattcgaaaattcaaggaggccggaatgctgctaacataatgattgatgaaagaacgcgattatggattaccagtattaaaagattaataacttttgaagaatggtataccagcaaccacgatagcttagcctaa

SEQ ID NO: 163 - DNA, 309 bp, Saccharomyces cerevisiae
atgtctagtgaaagagcctgtatgctgtgtggcatagtgcagaccacaaatgagtttaatagagatggttgtcccaactgtcagggtatttttgaagaggcaggtgtttctacaatggaatgtacgtcgccttctttcgagggcctcgtaggaatgtgtaagcctactaagtcgtgggtagcaaagtggctgagcgtagatcatagtatagctggtatgtacgccatcaaggtcgatggtagactaccagctgaggttgtggagctgttgcctcactacaaaccgagggatggcagtcaagttgagtaa

SEQ ID NO: 164 - DNA, 1719 bp, Saccharomyces cerevisiae
atgctatcaaaatcagaaaaaaatgtcgacctcttggcaggcaatatgagcaatttgagtttcgatgggcatgggactcctgggggcactggactatttcctaaccaaaatataacaaagaggaggacaaggcctgcgggtatcaatgattcaccttcgccggtaaaaccatccttttttccttacgaagatacttccaacatggacatagacgaagtatctcaacctgatatggatgtctcgaactctcccaagaagcttccaccaaagttttacgaaagagcaacttcaaataaaacacagagagttgttagtgtttgcaaaatgtactttctagaacattactgtgatatgtttgactatgtaattagtagaagacaacgcacgaagcaagttctagaatacctgcaacagcaaagccaacttccgaattctgaccagattaaactcaacgaagagtggtcctcttacttacaaagggaacatcaggttttgagaaaaagaaggttgaaaccaaaaaatagggattttgaaatgatcacacaagtaggtcaaggtggttatgggcaagtttatttagccagaaagaaagacacaaaagaggtgtgtgctttgaaaattttgaacaaaaaactattgtttaagctcaacgagacaaaacacgttttaactgaaagagatatcctaaccacgacaaggtctgaatggttagtaaaacttctgtatgcattccaagacctacaaagtttatacctggctatggaatttgtaccaggcggtgattttcgtacattattaataaatactagatgcttgaaaagtggccatgcgagattttacatcagtgaaatgttttgcgctgttaatgcgttacatgatttaggctatacacatagagatttaaagccggaaaacttcttaatcgatgccaagggacatataaaattaacagattttggtttggctgctggtacgatttctaatgaaagaattgaaagtatgaagataaggttagaaaaaatcaaggacctggaatttccagcgtttacagagaaatctatagaagatagaagaaaaatgtacaaccagttgagggagaaggaaattaattatgcgaactccatggttgggtctccggattatatggctttggaggttttagaaggaaagaaatatgactttactgtggattactggtcgttgggttgtatgctgttcgaaagtttagttggttatacaccatttagtggatcatcgactaatgagacctatgacaacttaaggcgctggaaacaaactttaagaagaccgagacaatctgatgggagggcagcattctccgatagaacttgggatttaataacaagattgattgccgatccaatcaatcgattaagatccttcgaacatgttaaacgcatgtcttattttgcagatataaactttagtactttgaggtcaatgatcccgccttttacaccccaactagacagcgaaactgatgccggttattttgatgacttcaccagtgaggctgacatggccaaatatgctgatgttttcaaaagacaagacaaattaacggctatggtagatgactcggcagtatcatcaaaacttgttgggttcactttccgacatagaaatggtaaacagggttccagtggcatcttattcaacggactggaacactcagaccccttttcaaccttttactag

SEQ ID NO: 165 - DNA, 924 bp, Saccharomyces cerevisiae
atggttcagcaactaagcctttttggatctattggtgatgacggctacgatttactaatttcaactttgaccacaatatcaggtaatcctccgctactgtataacagtttatgcactgtctggaaaccaaatccatcttacgacgtcgagaacgtgaactctagaaaccaattggttgaaccaaatagaataaaactttccaaagaggtgccattttcttacctgatcgatgaaacaatgatggataagccattaaactttagaatcttgaaatcttttacaaacgataaaatcccgcttaactatgctatgacacggaatatcttgcacaacacagttccgcaagtcaccaacttcaacagcacaaacgaagatcaaaacaacagtaagcatacagaagatactgtaaatgaaagtcgaaacagcgatgacatcatagatgtcgacatggatgcaagtcccgccccttcaaacgagtcatgttccccttggtcattgcaaatttcagatattcctgctgcaggaaacaatagaagtgtttcaatgcaaacgatagctgagactatcatattatcttcagctggcaaaaactcttcagtatcctcgctcatgaacggattgggttatgtattcgaatttcagtatcttacaattggtgtgaaattttttatgaagcatggtttaatacttgagttacaaaaaatttggcaaatagaagaagcaggcaattcacaaataacaagcggagggttccttttaaaagcatacatcaatgttagtagggggaccgatatcgatcgtataaactatacagagactgccttgatgaacttaaaaaaggaactacaaggctatatagagttaagtgtacccgatagacagtcaatggactcgagggtagcacatggaaatattctaatataa

SEQ ID NO: 166 - DNA, 2454 bp, Saccharomyces cerevisiae
atgagcactgaacaggtcggtagaaagaaatcgtatagatgggttagcgcatctcaagcaagttatgacggcgctggatgggacagtagtgacgaatatgactactcttccgaggatggcacaaaaggttcagaaattcacaagcaaaaaatatctaatttgcctagtttacccaagctcaattatacagatgttaatggtgagcatgatgaaaatacgggtgaaaataaagacagcaatgataataacgtcagtaagagtgatattagtcctagtgacaaggaagtaggctatctctcagatggggtgcctaaacttatggcttcaagagagtctgtggaattacaagcgaaaaaatccagcgagcactccaaatcagattatctttcgtcgacagcatctttgaaaagtccaagtgaaaacaaaaaatcaccacatacaaatagggctgtaaacgaagacctagacaatttgatagagcaaatatcaagggagatgacaccagaaattaggcaaacatctgattttcggcgtgattcagacagttgtgacgaaatccaaaacgaagcaccattaggggaagcggtaccctcttcatcatctcctgtagaagaagatgaaaaatcacattccttaggggtttcaatggatactaatgaagctgataccactttcaatacgccaacaaggaatggaaatgaacatttgagtagtgatggcgatgtttctgagcagaaagacgacgagtttaaagtttccgaaagaggctaccttgcagatattctgcctgcggagaaagaggagaatttacaacaagaagatgatggtgaagtcgagagctcaggagctttggaaaagaaagaaaaaagtgaagaaaagactagtattaggaataggaactctacatctagtgggcaagataaagtagcaaagccgaaacccgtagcaaatgaaacaaaaactagtgataatggatatcgaaattcttttttcaatgattatcagcattcgtcagactctgaagaagatgataacaacgaaggcaacagtggtagtagtgatgatgataatcgatcttcggttagtgataaacatgctgatattaaccggcaatcgaaacaacttgatacaactgacgatgatgcattgtcgtataccgaatcaataaaatattcgacaaatgagacagaagaagaagataatgaagataatgaatccatagaagacaaaaacgaagataatgaatccatagaagacgaaaatgaagatacagatagctataaattttctaatagggaaaaaggttccattctacttacgagtgatgaagaggaggaggaaaaaggtatgagctctgattcagatgaaggcagtctcaaggcgcccaaatcaggttatttctccaaaatgattggtaatgatgataaaggtgattcagcattgcaacctaatcaaatagacaccattgaaaataccaatttgtcaaatagcggttctgaattggaaaacagcgatggcagcgatgaggaagaccacataaatgaagacaaagttttggaagaaagttcagttaaagactccactgatgttgattcttggaagccggacagtgaggccttacgatcagggttcgtacaggatacagccaataagaaagcgccaccaggttacgtgatcgattctaacgggaagctagtagatttaacacctgctagtatgaaaccacgtgttgtatcgacttattcagagatggagagtacttgggatgcattcccttccaaaggcgaagatgacgatttggagacaatcagggacactaagactatatacgacaacaatacgatttataatgttcccggtttgattggtaaccaatcgaatttgccacctctaccgatggacgcacaggaacaattaaatgcaggaaacgataatagtacaacagataatgataatagtaacaatactgccaatgatttggcagcgcgcagtgcatctttcaaaagtgaaaacagaacagtatctcaaggcgaaatgacaagcgtccatgaacccagtactgaagaaatggcgaaattggggcagcagaataacttaccgaaattagatatgaataaactactgaacagtaagacttcgcacgccggtaagatagaacagttgagaaattataagcgtgagctagatgaatatgatacggggatacaaacatggataaattatacgctaaaatcatcctctaataaggataaggatttcatagctgaggagtataagcagcatagtcacgttagggaagcatatgcgaacgcggacgatttgagtaagaagcatactgtcattaacacggttgctagtgtcaaccaaaatgtaacgcacttaaggagaaaagttttccagcattctatgaaaccaaaggatttattcgcatcaataggtaagaaaaaactgtaa

SEQ ID NO: 167 - DNA, 786 bp, Saccharomyces cerevisiae
atgacgcaagaattcattgataaagtatcttcctacctgactcctgatgtggacattgcacctatttctcaaggtgcagaagccattgttttcacaacgactactcacccatatctcccaagagcaaaggattctcatcaaaagtatattatcaaatataggccaccaaagcgttatagacatccacaaatagaccaggcactaacaaaacatcgtacgttgaatgagtcgcgtttattggccaaattatacttgattccagggctatgtgtccctcaactgatagcgtgtgatccatacaatggattcatttggttagagttccttggagaagatcttcccggagggcacggttttagtaacctaaagaacttcctttggatgcatgaccaagatccatatagtgatcttgtagcaactacactacggaaagtggggcgccaaattgggttgttgcactggaatgactactgtcatggtgatttgacaagttctaacattgttctcgtgcgagatggtgcgagatggacgcctcatttgattgatttcggtctgggctcagtttcaaacctggtcgaagataaaggcgtcgatttatacgtcttagagagagctattttaagtacacattcgaagcatgcggaaaaatacaatgcttggatcatggaggggttcgaagaggtctatcgtgaacaaggtgcgaaaggtgccaagaaactgaaagaagttaccaaaagattcgaagaggtcaggttgcgtggtcgtaagagaagtatgctaggataa

SEQ ID NO: 168 - DNA, 5904 bp, Saccharomyces cerevisiae
atgtcaactgaatactctgctgattcatctaaatcatttatgattgcaatgcaatctatgattgatacttcacaaacctttaatctagatagatcgaaaatctctttaccagattttgatgacgagctaaaaaaagtccaaaaggatgaaccaaatcaaagaactgaactaactgttctctcacaagatagaaatgattgggatgatatatttgaggagtttaaagacatttcatttgctcaactgcaatctataattgattcatacaaaacaaagaatgcagttgcagtatataaaaaaattggaaaactaatcaatgaagctgaaacgacactaagcagcaatgtattattagaaactgtactgcaaatggtttataaacatcaaaagcaagagttagagaaagaattgttggattttttaggaacagggaatatagaccttgtttctctgttacttcagcatagaagaatgatcgttgctacacctatagaaacgacgattctgctaataaaaaatgcggtcaattcaacgccagagttcctgacacagcaagatatcaggaatcaagttttggaaagtgcagaggatgccaagaataggaaattaaatcccgccactaaaataatcaagtacccacacgtcttcagaaaatatgaagcaggatccactacagctatggcctttgccggtcaaaaattcactttgccagtagggactacaaggatgtcgtacaatacccatgaagaaattataattccggcagcagatcaggcatccaacaagaactatctctatacaaaactattgaaaataagtgatttggatcatttttgcaagactgtatttccctatgaaactttaaaccaaatacaatcccttgtttaccccgtagcttataaaacaaatgaaaatatgctaatttgtgcacctactggtgcaggtaaaacagacattgcattactgacaataattaatacaataaaacaattctccgtggtcaacggggaaaatgaaattgatattcaatatgatgattttaaagttatttatgttgcccctttgaaagcactcgctgctgaaatcgtcgacaagtttagtaagaaattggcgccttttaatatacaagtaagggagttaacaggcgatatgcaattgacaaaggcggaaattttagccactcaagtaatagtcacaacccctgagaaatgggacgttgtcacccgtaaagcaaacggcgacaacgatttggtttcaaaggtcaaattactaattattgatgaagttcatttactgcacgaagatagaggtccggttattgaaactttagttgcacgtaccttgagacaggtagaaagctctcaatcaatgattcgtattatcggtctatcagctacattacccaattttatggatgtagcagatttcttaggagtaaatagacagatcggaatgttctattttgatcaatcattccgtccaaagcctctagagcagcaacttttgggctgtagaggtaaggcaggtagtagacaaagtaaagagaatatcgataaagtcgcttacgacaaattgagtgaaatgattcaaagagggtaccaggtaatggtatttgtccactcaagaaaggaaactgttaaaagtgcaagaaactttatcaaactagcagaatctaaccacgaagttgacctttttgcacctgaccctatcgaaaaggataaatattctagaagtttagtgaagaacagagacaaggatatgaaggagatttttcaatttggttttggtatccatcatgctggtatggcccgttcagatagaaatcttactgaaaaaatgtttaaagacggtgctatcaaggtactgtgttgtactgcaacattagcatggggtgttaatttacctgctgattgtgttatcatcaagggtacacaagtatatgattctaaaaagggtggattcatagatctaggtatttcagatgttattcagatttttggcaggggtggtaggccgggcttcggttccgcaaatggtacgggtattttatgtacttccaatgatcgtctggatcattatgtttccttaattactcaacaacaccctatcgaatctagattcggttccaagttggttgataacctgaacgccgaaatttcattggggagcgtgacaaatgtagatgaagcaatcgaatggcttggttacacatacatgtttgttcgtatgagaaaaaatccttttacttacggtattgattgggaggaaattgcaaatgacccgcaattatatgaacggaggagaaaaatgatcgtagtagctgcaaggcgattacatgctctccaaatgattgtgttcgatgaagtgtcaatgcatttcattgcaaaggatttgggcagggtgtcctctgatttttatcttttgaacgaatccgtcgagatattcaatcaaatgtgtgatcccagagccaccgaagctgatgtattgtccatgatcagtatgagcagtgaatttgacggtataaaatttagagaagaagaatccaaggagttgaagagactttctgatgaatctgttgagtgccagattggaagccaacttgacacccctcaaggtaaagcaaacgtgctattacaggcttatatttcccagactagaatttttgactcagcactatcatccgattcgaattatgttgctcagaactctgttagaatttgtagagcattgtttttaataggtgtcaatagaagatggggtaaattttcaaacgtcatgctaaatatttgcaaatctattgaaaagaggctctgggcctttgatcaccctctctgtcagtttgatttgcctgaaaatattatcagacgcatcagagatactaaaccttccatggagcatcttttagaattagaggcagatgagctcggagagctggtgcataataagaaggccgggtctaggttatataaaatattgagccgatttccaaagattaatatagaagctgagattttccccattacaacaaatgtaatgaggatacatattgcattaggtccagactttgtatgggattcccggatacatggtgatgcacagtttttttgggttttcgttgaagaatctgataagtcgcaaatccttcattttgaaaagttcatcttaaacagaaggcagttgaataatcagcatgaaatggacttcatgatcccattgtcagaccctttgcctccgcaggttgtcgtaaaggttgtgtcggatacttggattggctgtgaatcgacccatgccatctcttttcaacatttaatcagaccatttaacgaaactttacagacaaaattgttgaagttacgtcctctacctacatcagccctccagaatcctttaattgagtcaatttatcctttcaagtatttcaatcccatgcaaactatgacattttatacactttacaataccaatgagaatgcgtttgttggctcgcctacaggttctggtaaaacgattgtggctgaattggctatatggcatgccttcaaaacatttcctgggaaaaagattgtttatatcgcaccaatgaaagcattagtacgtgaaagggttgatgactggagaaagaaaataacaccagtgaccggagataaagttgtagagttaaccggtgattctttgccagatcctaaagatgtgcatgatgccaccattgttattacaactccagaaaagtttgatggtatttcccgtaattggcaaactcgtaagtttgttcaagatgtatctttaattatcatggatgaaattcatttgttggctagtgatcgtggtcctatcttagaaatgattgttagtcgtatgaattatatttcctctcaaaccaagcaaccggtcagactgctaggtatgtcaactgctgtttcaaacgcctacgacatggctggctggttgggagttaaggaccacggactgtataacttcccgtcaagcgtccgtccggttccattgaaaatgtacattgatggatttccagataatttggcattctgtcctctgatgaaaacaatgaacaaacctgtgtttatggccattaaacaacattcacctgataaaccagcattaatatttgttgcatcgcgtagacaaactagactaacggctttagacttgatccacttatgtggaatggaagataatccacgtaggtttttgaatatagacgatgaggaggaactgcaatattacttatcacaggtcactgacgacactttaaagttatcactccagttcggtattggattgcatcatgcaggtctagtgcaaaaagatcgttctatttctcatcagttgtttcaaaaaaacaaaattcagattctgatcgctacatcaaccctagcgtggggtgtcaatttgccagctcacttggttatcatcaaaggtacacaattttttgatgcaaaaattgaaggatatagagatatggatttaacagatatcttgcaaatgatggggagagctggtagaccggcttatgacactacaggtactgctatcgtttatacaaaagagtccaaaaaaatgttttataaacatttcctaaacgttggttttcctgtcgagtcctccttgcacaaggtattagatgatcatttaggtgcagaaataacgtcaggtagcataactaataagcaagaagcgttagattttttgagttggacatttttgtttcgtagagcgcatcataacccgacatattatggcattgaagatgatacaagtacagcaggagtcagtgaacatttgagttctttgatagatagcacattagaaaatctgcgagaatcacaatgtgttttattacatggagatgatattgttgctacgccattcttaagtatatcctcatactactatatttcacatttaacgatacgccaattattgaaacagatacatgatcatgcgacttttcaagaggtattacgatggttatcgttggctgttgagtataatgaattgcctgtaagaggtggtgaaattatcatgaatgaagaaatgtcgcagcaatcgaggtactccgttgaaagcacttttaccgatgaattcgaattaccgatgtgggatccacatgtaaaaacatttttattacttcaagctcatttaagccgtgtagatttaccgattgcagattatatccaggataccgtatcagttttggatcaatctctacgtattctacaggcttatattgatgtcgccagtgaattgggttactttcatacggtattgaccatgataaagatgatgcaatgcataaagcaagggtattggtatgaagacgatcctgttagtgtacttccaggattacaactgagaagaatcaaagattacacttttagcgagcaggggttcattgaaatgacgccgcagcaaaagaagaaaaagctgttaaccttggaggaaataggaaggtttggatataagaaattattaaatgtttttgatcaactaacttttggaatgacagaaagcgaagatacgaaaaaacgtttcgtcagtgtctgccaaagattaccggttttagaaggcatgaaatttgaagaacaggaaaataacgaggtgttaacattttatagcaagcacctttctagtaagcataataatgggtttgaagtatattgtgataaattccctaagatacaaaaagaactatggtttttaattggacacaaaggcgatgaactgctgatgattaaaaggtgtcaaccaaaacaaatgaataaagaggtcataatacattgtgatctatttattcccgaagaaattcgtggcgaagaattgcaattttcccttattaatgacgctttaggattacgttatgatatggtccataaactaatctcttag

SEQ ID NO: 169 - DNA, 999 bp, Saccharomyces cerevisiae
atgggtgtcatcaagaagaaaagatcgcaccatggaaaggcttcgcgccaacaatactactctggggtacaggttgggggagtaggcagcatgggcgccataaacaataacatcccgtcgttgacgagcttcgcggaggaaaacaactatcagtacggatacagcggctccagtgccggcatgaatggcagatcgcttacgtacgcgcagcaacagcttaataagcaaagacaggacttcgaacgtgtacgacttagaccagaacagctcagcaatatcatacatgacgagagcgacacgatatcgttccgatccaaccttttgaagaactttataagctcgaacgacgcatttaacatgctgagtttgaccacggtaccgtgcgacagaattgagaagtccagattgttcagtgaaaaaacaataagatatctcatgcaaaaacaacacgaaatgaaaacacaagcggcagagttgcaagaaaagcctctgacgccacttaaatacacaaaacttatagctgcggcagaggacggaagccgtagcacaaaggatatgatagatgctgtcttcgagcaagatagtcatttgaggtaccagccggacggcgtggtcgtacatcgtgacgatcctgcgctggtgggtaaactccgcggagatctccgcgaagcgccggcggactactggacgcatgcttatagggatgttttggcgcaataccacgaggccaaggagcgtatcaggcagaaggaagtaactgcaggtgaagcacaggacgaagccagcttgcagcagcaacagcagcaagatttgcagcaacagcaacaagtagtgactacagttgcctcgcaaagtcctcatgcaactgcaacggaaaaggagccagtacccgccgtggttgacgacccactggagaacatgttcggagattattccaatgagccgttcaacaccaatttcgacgatgaatttggagatcttgatgctgtatttttttaa

SEQ ID NO: 170 - DNA, 717 bp, Saccharomyces cerevisiae
atgcctattaatattcgcagagcgacaatcaatgacattatatgtatgcaaaatgccaaccttcataacctacccgaaaattatatgatgaaatattatatgtatcatattctctcgtggccagaggcttcgtttgttgctactaccaccactttggactgtgaagatagtgatgaacaagatgagaacgacaaattggaattgaccttagatgggaccaatgacggcaggacaatcaagttggatccaacatacttggctccgggcgaaaaattagttggatacgttctcgtgaagatgaacgacgaccccgaccagcaaaacgagccaccaaatggtcatattacttccttaagtgtcatgagaacctatagaaggatggggattgccgaaaacttaatgagacaagctttatttgcgttgagagaagttcatcaagcggaatatgtttccttgcatgtgagacaatctaatagagcagctttacacttgtacagggacacattggcgtttgaggtgttgagtatcgagaaaagctactatcaggatggtgaagatgcatacgccatgaagaaggttttaaaattagaggagttgcagataagcaacttcacccatcgccgtttgaaagagaatgaggaaaaactagaagacgatctagaaagcgatctactagaggatatcattaagcaaggcgtaaatgatatcattgtataa

SEQ ID NO: 171 - DNA, 1455 bp, Saccharomyces cerevisiae
atggctgataagatagagaggcatactttcaaggtcttcaatcaagatttcagtgtagataagaggtttcaacttatcaaagaaatagggcatggagcatacggcatagtgtgttcagcgcggtttgcagaagctgccgaagataccacagttgccatcaagaaagtgacaaacgttttttcgaagaccttactatgtaaaagatccctacgtgagctaaagcttttgagacatttcagaggccacaaaaatattacatgtctttatgatatggatattgttttttatccagacgggtctatcaatggactatatctttatgaggaacttatggaatgtgatatgcaccaaatcatcaaatccggtcaacctttgacggatgctcactatcaaagtttcacataccaaatattatgtggtttaaagtatattcattctgcagatgtcttgcatcgtgatttgaagcccggcaatttgcttgtcaatgcagattgtcaattgaaaatctgtgattttgggttagctagaggttattcggagaatcctgtcgaaaacagtcaatttttgacggagtacgtggccactagatggtatagagctccggaaataatgttgagttaccaaggatataccaaggcgattgacgtatggtcagctggctgtattttagcggagtttcttggtggaaagccaatcttcaaaggaaaggattacgttaatcaattgaatcaaatattacaagttttagggacacccccagacgaaactttaagaaggattggttctaaaaatgttcaggactacatacatcaattaggtttcattccaaaagtaccttttgtcaatttatacccaaatgccaattcacaagcattagacttattggagcaaatgctcgcgtttgaccctcaaaagagaattaccgtggatgaggccctggagcatccttacttgtctatatggcatgatccagctgacgaacctgtgtgtagtgaaaaattcgaatttagttttgaatcggttaatgatatggaggacttaaaacaaatggttatacaagaagtgcaagatttcaggctgtttgtgagacaaccgctattagaagagcaaaggcaattacaattacagcagcagcaacagcagcagcaacagcaacagcaacagcaacagcagccttcagatgtggataatggcaacgccgcagcgagtgaagaaaattatccaaaacagatggccacgtctaattctgttgcgccacaacaagaatcatttggtattcactcccaaaatttgccaaggcatgatgcagatttcccacctcgacctcaagagagtatgatggagatgagacctgccactggaaataccgcagatattccgcctcagaatgataacggcacgcttctagaccttgaaaaagagctggagtttggattagatagaaaatatttttag

SEQ ID NO: 172 - DNA, 336 bp, Saccharomyces cerevisiae
atggccctgtggggtagaagtgcctacagacaaaaaaccgttacgtcccgcctcaccaagcatcgacacacaagccctctcaacctccttaatttctttatctttttttcccttcacctctgtgctctttttttagccacagctgtgcattacgcttgctttgcttgttttgttctctttcgccatgccatattactacttttctacttactggcacgaggccgcgcaagccagatccaagcacgccagaaagtgcggtgtactggtgcaaccttttatcgatttctcatcatatcgctatcgcaacgggcatgggcgacgaaaaagccaatctaa

SEQ ID NO: 173 - DNA, 4437 bp, Saccharomyces cerevisiae
atgccctttttgaggaaaatagcggggacagcacatacacattctaggtctgattcgaactcatctgtgaaattcggccatcagccgactagttcggtagcatcaaccaaaagttcaagcaaaagccctcgtgcaacatctcgcaaaagcatttatgatgatattagaagccaatttcccaacctaacccccaactctacctcttctcagttttacgaaagcacgccagttatcgaacaatcctttaattggacgacagatgaccacatctcagctggaacgcttgaaaacccaacgagctttacaaacagttcttataaaaatgacaatggacctagtagcctctctgattcgaggaaatcctccggtggcaatagcgtaaatagtttgtcctttgacaagctaattctatcgtgggatcctacagaccctgatgaatggacaatgcatcgcgtcacctcatggtttaaatttcatgattttccagaatcctggatattgtttttcaaaaagcatcaattgtttggtcacagatttataaagttgcttgcatatgataatttcgctgtttatgaaaagtatttgccgcagactaaaactgcttcatataccaggtttcagcagttattgaaaaaaacaatgaccaagaacgtaacaaatagccatattcgtcagaagagcgctagcaaacttaaaagttccaggtcttccagcgaatcgatcaaatcaaaattaaaaaatagtaaatcgcaagaggatatttcaaattctagatcaacgtcagaatctgcattgagcccaacaaaatcgggcccttccaagaccgatgaaaagaattttttacattctacttcaacacaccaaaaaaccaaaagcgcaagttcactatacagaagaagttttatatccctaagaggctcatcatcgagcaatgcttcctcagcaaaatcaccttcaaacatcaagttaagtataccggctcggccgcactcaattattgaatctaacagtacacttaccaaatcggcgagcccacctgcatctccttcgtatcctagcatatttagaagacatcacaaaagtagttcatctgagtcgtcattattaaattccctttttggtagtggaataggcgaggaagctccaacaaagcctaatccacaaggtcatagtctgtctagtgaaaatttagctaaaggaaaatctaaacactatgaaactaatgtgtcttcacctttaaaacaatcttcactacccacttcggatgataaaggtaatttatggaataaattcaaaagaaagagccaaataggggttcctagcccaaatacggtagcttatgtaacgtctcaagaaactccatccttaaaatcgaattcgagtactgctaccttaaccgtacaaacggcagatgtaaatataccatctccatcttcatcaccaccgccaatacccaaaactgcaaacagaagtttggaggtcatcagcacagaagatacacctaaaatttcttcaaccacggcgtcttttaaagaaacgtatcctgattgtattaatccagacaagacagttccagtgccggtaaataatcaaaagtatagtgtaaagaactttttactggaccaaaaattttatcctctgaagaaaacagggttaaatgatagtgagaataaatatattctggttaccaaagataatgttagttttgttccgctaaacttaaaaagtgtagcaaaattatccagtttcaaagaatctgctctcacaaaattgggaatcaatcacaaaaatgtcactttccatatgacagactttgattgcgatattggtgctgcaattccagatgatactttggaatttttgaaaaaaagcttgtttttgaacacttctggaaaaatttatatcaaagaccaaatgaagcttcaacaaaaaccgaaacctgctcctctcacctcagaaaacaatgttcctttaaaatcggtgaaaagtaagagttcaatgaggtccggaacaagcagtctgatagcatcgacagatgatgtttccattgtcacttcgtcttctgacataacatcatttgatgaacatgcatcaggaagtgggcgcaggtacccccaaaccccgagttattactatgacagagtttccaatactaatccaactgaagaattgaattattggaatattaaagaagttctttctcatgaggaaaatgcaccaaaaatggtttttaaaacaagtccaaaattagaactcaacctaccagataaaggaagtaaattaaatattcctacccccataacagaaaatgaaagcaagagtagttttcaagtgctaagaaaagatgaggggactgaaattgatttcaatcatcgtagggaatcgccttatacaaaaccagaactggcaccaaaaagagaagctcccaagcctcccgcaaatacttctcctcagaggaccttatcaacttctaaacagaataaaccgatccgcctagtgagggcaagtacaaaaatttcgagaagcaaaagatcgaaaccattgccgccacaattattatcatctcctatagaagctagcagctcgtctcctgattcgcttacttcctcatatactcctgcttcgactcatgttttgataccgcaaccttataagggtgcaaacgatgttatgcgtaggttgaaaacagaccaggactcgacgagtacttccccatctttgaaaatgaaacagaaagtgaatcgctcaaattcaactgtatcgacttcaaattcaattttctattctccttcaccattgttaaaaagaggtaactcaaaaagagttgtttcgtcgacatctgcggccgatatatttgaagagaatgacataacattcgcggatgctccgccgatgtttgacagcgatgatagtgatgacgattctagttcatccgatgacattatctggtccaagaaaaaaacagctcctgagactaataatgaaaacaaaaaggatgagaaaagcgataacagttctacgcattctgacgaaatattctatgattctcaaacgcaggacaaaatggagagaaagatgacctttagaccatctccggaggtcgtttatcaaaatttagagaaattcttcccaagggctaacttagataagccaatcactgaaggaatagcttcaccaacatctccgaaatccttagacagcctactttcaccaaagaatgtggcttcatcgagaactgagccaagcactccttcccgtcccgtccctcctgatagctcatacgagttcatacaggatggacttaacggtaaaaataaaccattgaatcaagctaagacacctaaaagaacaaaaaccataagaaccattgcacatgaagctagtttagcaagaaaaaactctgtaaaactaaaaagacagaacaccaaaatgtggggtacaagaatggtcgaagtgaccgaaaaccatatggtgtcaattaataaagccaaaaattcgaaaggtgagtataaggaattcgcctggatgaagggtgaaatgatagggaagggatctttcggtgctgtttatttatgtttaaacgttactacaggtgagatgatggccgttaagcaggttgaggtccccaagtatagctcacaaaatgaagccattctaagtaccgtggaagcattaagatctgaagtgtccacgttaaaagatttagatcatcttaatattgttcaatacttaggttttgagaataaaaacaatatttacagtttgtttttagaatatgttgctggtggctccgtgggatccttgattagaatgtatggaagattcgatgaaccgttgatcaaacatttaacaacacaagtattaaaaggattggcatacctacactcgaaaggtattctccacagggatatgaaggcagacaacttacttttggatcaagatggtatctgcaaaatcagtgacttcggaatttcaagaaaatcaaaggacatatactctaattcggatatgaccatgcgaggaacagtcttctggatggctcctgaaatggttgatacaaagcaaggctacagtgcaaaagttgatatatggtctctgggatgcatcgttctggaaatgtttgctggtaagcgcccgtggtccaacttagaagtcgtcgcagccatgttcaaaattggaaagtcaaaatcggcaccaccaattcctgaggacactttaccattgatatcgcaaatcggacgaaattttctggacgcatgcttcgagataaatccagagaaaaggccaaccgctaacgagcttctttctcatccttttagtgaagtaaatgaaacattcaatttcaaatctaccagactcgcgaagtttataaagtcaaatgataagttaaactcttcaaaattaaggataacctctcaggagaataaaactgaatag

SEQ ID NO: 174 - DNA, 3708 bp, Saccharomyces cerevisiae
atgcattttagaagaacgatgagtagagtgcccacattggcatctcttgaaatacgatataaaaaatctttcggccataaatttcgtgattttattgctctatgtggtcactattttgctccagttaaaaaatatatcttccccagttttatcgcggttcactacttctacacgatatccctgacattaataacttcaatcctgctatatcccattaagaataccagatacattgatacattgtttttagcagcgggcgcagttacacaaggtggcttaaatactgtggatatcaacaatctaagcttataccaacaaattgttctgtatatcgtatgctgcatatcaacaccaattgcagttcatagttgcttggcatttgtacggctttactggtttgagcgctacttcgatggtattagagactcttctagacgaaattttaagatgagaagaacgaaaacaatcttagaaagggaactaacagcaagaaccatgaccaagaatagaacaggtacccaaagaacgtcttatcctaggaaacaagctaaaacagatgatttccaagaaaaattgttcagcggagaaatggttaatagagatgagcaggactcagttcacagcgaccagaattctcatgacattagtagggacagcagcaataataatacgaatcacaatggtagcagtggcagtttagatgatttcgttaaggaagacgaaacggatgacaatggagaatatcaggagaacaactcctactcgacggtaggtagttcgtctaacacagttgcagacgaaagtttaaatcagaagcccaagccaagcagtcttcggtttgatgagccacacagcaaacaaagacccgcaagagttccctcagagaaatttgcaaaaagaaggggttcaagagatattagcccagccgatatgtatcgatccattatgatgctacaaggtaagcatgaagcaactgctgaagatgaaggtccccctttagtcatcgggtcccctgcggatggcacaagatataaaagtaatgtcaataagctaaagaaggccaccggcataaatggtaacaaaatcaagattcgagataagggaaatgaaagtaacactgatcaaaattccgtgtcaagtgaagcaaacagtacggcgagcgtttcggacgaaagctcgttacacacaaattttggtaacaaagtaccttcattaagaacaaatactcatagatcaaattcgggcccgatagccattactgataacgcagaaacagacaaaaagcatgggccatcaattcaattcgatataactaaacctcctagaaaaatttcaaaaagagtttcaaccttcgatgatttgaacccaaaatcttccgttctttatcgaaaaaaagcatcgaagaagtacctcatgaaacattttcctaaagcgcggcgaatacggcaacaaattaagagaaggctttctactggttcaattgagaaaaacagcagtaacaatgtttcagatagaaaacctattactgatatggatgatgatgatgatgacgatgacaacgacggcgataacaacgaagaatactttgctgacaacgaaagcggcgatgaagatgaacgagtacagcagtctgaaccacattctgattcagaactcaaatcgcaccaacaacagcaagaaaaacaccaactgcagcagaacctgcaccgcatgtataaaaccaaatcatttgatgataatcgttcaagagcagttcctatggaacgttccaggaccatcgatatggcagaggctaaggatctaaatgagctcgcaaggacgcctgattttcaaaaaatggtctatcaaaattggaaagcccatcatagaaaaaaaccgaactttaggaagaggggatggaataacaagatatttgaacatggtccctatgcatctgacagcgatcgcaattatcctgataatagtaatactggaaacagtattcttcattacgcagagtctattttacatcatgatggctctcataaaaatggaagcgaagaagcctcttccgactctaatgagaatatctattccacgaatggaggaagcgaccacaatggtcttaacaactatcctacttacaacgacgatgaagaaggctattatggtttacatttcgataccgattatgacctagatcctcgtcatgatttatctaaaggcagtggtaaaacgtatctatcatggcaaccaactattggacgtaactcaaacttccttggattaacaagagcccagaaagatgaattaggtggtgtcgagtacagagcaatcaaacttttatgcaccatattggttgtctactacgttggatggcatattgttgcttttgttatgttagtaccttggattattttgaaaaagcattatagtgaagttgttagagatgatggtgtttcacctacatggtggggattttggacagcaatgagtgcatttaatgatttaggtttgacattaactccaaattcaatgatgtcgtttaacaaagctgtatacccattgatcgttatgatttggtttatcattatcggaaatacagggtttcccatccttcttagatgcatcatttggataatgtttaaaatttctcctgatttatcacagatgagagaaagtttaggttttctcttagaccatccacgtcgttgtttcaccttgctatttcctaaggcagctacatggtggctacttttaacgcttgcaggattgaatataactgattggattttatttattattctagattttggctcaacagttgtgaaatcattatcgaaaggctatagagtccttgtcggcctgtttcaatctgttagcacaagaactgctggattcagcgttgtcgatttaagtcaactgcatccttctatccaagtctcctatatgctaatgatgtatgtctccgtattaccattggccatctctattcgacggacaaatgtttacgaggagcaatctttaggactatatggagatatggggggagaaccagaagatacggatactgaagacgatggtaacgatgaagatgacgacgaggaaaacgagagtcacgaaggtcaaagtagtcaaagaagtagttcgaacaacaacaacaataacaacaggaaaaagaaaaagaaaaagaaaactgaaaatccaaatgaaatatctacaaaatcctttatcggtgcccatttaaggaaacagctttcatttgacttgtggtttctatttttagggttatttatcatttgcatttgtgaaggggacaagataaaggacgtacaagaaccaaactttaatatatttgcaattctttttgaaattgttagcgcttacggtacagttgggctatcgctaggttatccggacaccaaccaatcgttttcaagacagtttactacattatctaagttggtgatcatagctatgctgatcagaggcaagaatagaggtctaccatactcactggatcgtgcaattatcttgcctagtgatagacttgaacatattgaccaccttgagggcatgaaattgaagagacaggctagaaccaatacagaagacccaatgacggaacatttcaagagaagtttcactgatgtgaaacatcgttggggagctcttaagcgtaagaccacacattcccgaaatcctaaaaggagcagcacaacgctctaa

SEQ ID NO: 175 - DNA, 513 bp, Saccharomyces cerevisiae
atgtttttaacgtcaccatttgaatcctgtattgtgctgtcttccttaatagcaggcttattgttttcattgtctaccgggttcgtaggtatactaggagtgtttgcctcattgttcgaaacagaactctcggtttcgccaaagagattatcactatcctcattatcttggccaaagacgttctgggccctgctttcttctgtagaaggtgtttcctgggaatctagcttattcgcttgcatagtcggctgctgctttgccgttaccgttatcgcctcactttctgcatcgcgtgtgttcggaacagttgcttcatccttcagagactcatcttgttgctgcgattcttcaccagcagtatctgtattggcaacccctgccacagctgcattagcgttgctatcactattgttgtcattgccatgttggtccacgtccacggaagcgtttaccgtggacccctcacccagtgtattctccatgcttgcaaaccgtatcacaattggtctctga

SEQ ID NO: 176 - DNA, 372 bp, Saccharomyces cerevisiae
atgaaactaccggtagcacagtacagtgcaccagatggtgtggaaaaaagttttgcaccaatacgcgatgaccctcgatacatgaccacagagggaaggacaactgggcccagtgaccatgtgctaaacgctggccaaatcgatagagacaaaccttcagaaccggaacgcacgaaagatggctcacaactgacatacttaggccagctgcgcacgcagctgacggggctacaggacgatattaacgagtttttgacaggaagaatggaattggcaaaaaataagaagaaagccggcgcagacgagaagcggatccaggaagagattaaccagctattagatggtggtgacggtgacgaagatgctgtttag

SEQ ID NO: 177 - DNA, 3456 bp, Saccharomyces cerevisiae
atggatcaggataacaacaaccacaatgacagcaataggctgcacccacctgatatacatccaaatttgggccctcaattgtggctgaatagtagcggtgattttgacgacaacaacaacaacaacaacaacaacaacaataataatagcacaagaccacaaatgccatcacgaactagagaaacggcaacttcggaaagaaatgcaagtgaggttagggatgcaacgctaaataatatctttaggttcgatagtattcaacgggaaacgcttttgccaaccaacaacggacaaccgctaaatcaaaacttttcgctgacatttcaaccacaacagcaaacaaatgcgctgaacgggattgacataaacactgtgaacacaaaccttatgaatggtgtcaatgttcaaatagatcaacttaatcgattgttaccgaacctaccagaggaagaacggaagcaaatccacgaattcaagctaatagtgggcaaaaaaatccaagagtttctggttgttatagagaaacgtagaaaaaaaatactgaacgaaattgagctagacaaccttaaactaaaggagctacgtattgataactccccacaagcaattagttatttgcataaattacaaagaatgaggcttagggcgctagagacagaaaacatggaaattagaaatttaaggctaaaaatattaacaattatagaagagtacaaaaagtcattatatgcatactgccattccaagctaagaggtcaacaagtggaaaatccaacagataatttcatcatttggataaactccatagatactactgaatcatctgacttgaaagaagggctacaagatctttcgagatattcaaggcagttcataaataatgtgctttcgaatccatcaaatcaaaacatatgtacgagtgtcacccgaagatcacctgtgtttgccctaaacatgctaccctcggaaatattacacttaatattagataaacttaaccaaaaatatgatattgtaaaattccttaccgtttccaaactctgggctgaaataattgtgaagatactttattacagaccgcacatcaacaaaaagagtcaattagacttgtttttaaggactatgaagttaacttctgaagaaactgtattcaactatcgtttaatgatcaaaagattaaatttttcatttgttggtgactacatgcacgatacagagcttaactattttgtcggatgtaagaatttggagcgactaactttagtattttgcaagcatataaccagtgttccaatatcggctgttttgagaggatgtaaatttctccaaagtgtggatatcactggaataagagacgtttccgatgacgtatttgataccttagcgacatattgtcccagagtacagggcttttatgttcctcaggcaaggaatgtaacattcgattcactgcggaatttcatagtccattccccgatgttgaaaagaataaaaatcacagcaaacaataacatgaatgacgaattagtagaactattagccaacaaatgccctttgcttgtagaggtcgatataacattaagtccaaatgtcactgattctagtttgttaaaactcctcactaggttagttcagctgagggaattcagaataactcataatacgaatattacggataatcttttccaggagctttctaaagtagttgacgatatgccctctttaagattgattgatctttctggatgtgaaaatattacagataaaactatagaaagtatagtcaatttagcccctaaattacgtaatgtttttctaggcaagtgtagccgaattacagatgcatcgttgttccaattatcgaagctgggcaaaaacttgcaaacagtgcattttgggcactgtttcaatataactgataacggggtaagagcactctttcattcatgtacaagaatacagtatgtggactttgcgtgctgtacgaatttaaccaatagaactctttatgaactagcagacttaccaaaattaaagagaattggccttgtcaaatgtacgcaaatgactgacgagggtttgttgaatatggtttccttgcgaggccgaaatgatactttagaaagggtacatttatcttactgttctaatttaacaatatatccgatatatgagcttctaatgtcttgcccaaggctctcacatttgtctttgactgctgttccgtcatttttacgccccgatataacgatgtattgcaggcctgcaccctcagactttagtgaaaatcaacgtcaaatattctgcgtattttcagggaaaggtgttcataaacttcgccattatttagtaaatttaacgtcgcccgcttttggaccacatgtcgatgtaaatgatgttttgacaaaatatattagatccaagaatttgatatttaacggtgaaacacttgaagatgctcttaggagaatcataactgatttaaatcaagattccgctgcaattatagctgctacaggattaaatcaaatcaacggtctaaataacgattttcttttccagaatatcaattttgaacgaatagatgaagtattcagttggtatctcaatacttttgatggcattaggatgagctcggaggaagttaactcactattattgcaagtaaacaagacgttttgtgaagatccatttagtgatgtggacgatgatcaagattatgtcgtagcacctggtgtaaaccgggaaattaacagtgaaatgtgtcatattgttagaaaattccatgagttaaatgatcatattgatgatttcgaggtgaatgttgctagtttggtaagagttcagtttcagtttactggttttttacttcatgaaatgactcaaacctatatgcaaatgattgaattaaacagacaaatttgtttagtacaaaaaacggttcaggaatcgggcaacatagattaccaaaaagggcttttaatatggcgacttttattcattgacaaattcattatggtggttcagaagtacaagctctccaccgttgttttgagactatatttaaaagataacataacattgttaaccagacaaagagaactattaatagcccaccaaagatcagcatggaataacaataatgacaatgacgccaaccggaacgccaacaacatagtgaatattgtatcggatgctggggcaaacgatacaagtaacaatgaaactaacaatggtaatgatgacaatgaaacagaaaatccaaatttctggcgtcagtttggcaatagaatgcaaatatcacctgaccagatgaggaatctccaaatgggacttcgtaatcagaacatggttaggaacaataacaacaacacaattgacgaatcaatgcctgacactgccattgattctcaaatggatgaagcatcaggaacgcccgatgaagatatgttataa

SEQ ID NO: 178 - DNA, 609 bp, Saccharomyces cerevisiae
atgtctgcattacctccagtatactcattccctccactatatacccgtcaacccaattctttaacaaggagacaacagataagtacgtggatagacatcatatcacaatattgcaagaccaagaaaatttggtacatgtctgttgacgggactgtaatcaatgacaacgaactagatagtggaagtacagataatgacgattcaaaaaaaatctcgaaaaatctttttaataatgaggacatccaaagatctgtatcacaggtatttattgatgaaatttggtcgcaaatgaccaaagaaggcaaatgcttaccgattgaccaaagtggtaggagaagcagcaataccaccactacgagatattttatattatggaagagcttagacagttgggcttctttgatactacagtggttcgaagactctggaaaactgaatcaagtcattacgttatacgaattgagtgaaggtgatgaaacagtcaattgggagttccatagaatgcccgaaagcttgctctattattgtttgaagcccctatgcgatagaaatagggccacaatgctgaaagatgaaaatgataaagtaattgccattaaggttgtttaa

SEQ ID NO: 179 - DNA, 699 bp, Saccharomyces cerevisiae
atgagtttgtttgagtgggtatttggaaagaatgtcactccgcaagagaggttaaaaaaaaatcaaagggctttagaaaggactcagagggagcttgaaagagaaaagagaaaattggaactacaagataaaaaacttgtatcagaaattaaaaaatcggcaaagaatgggcaagttgcagctgcgaaagttcaagcaaaagatttagtaagaacccggaattacatccaaaaatttgacaacatgaaagctcaacttcaggcgatatcattgagaatacaggccgttcgaagtagtgaccaaatgacacgttctatgagcgaggccactggtcttttggctggaatgaacagaacaatgaatctacctcagttgcaaaggatatcgatggagtttgaaaagcagagtgatttgatgggtcaacgacaggaattcatggacgaagctattgataacgtcatgggtgatgaggtggatgaagacgaagaagctgacgaaattgtaaataaagttcttgatgagattggagtggatttgaattcacagttgcaaagcacgcctcaaaatctggtttctaatgcgccgatcgcagaaacagcgatggggattcctgaacctattggcgccggatcagaatttcacggtaatcctgacgatgacttgcaagctcggttgaacactttgaagaagcagacttga

SEQ ID NO: 180 - DNA, 1587 bp, Saccharomyces cerevisiae
atgtcctacaataatggcaatacttattcaaagagttatagcagaaataataagagacccttgttcggaaagaggtcgccaaatcctcagtccctagcgagaccaccgccaccaaagagaatacggactgatagtggttaccagtcgaatatggacaatatatcttctcatagggtaaattcaaatgaccaaccaggccacacgaaaagtcgtggtaacaataatttatctcgctataacgatacatcctttcaaacaagttccagatatcaaggttcaagatacaacaataataatacgtcgtatgaaaataggcctaaaagcataaaaagggatgaaacaaaggctgaattcctgtctcatttgccaaaggggcccaaatctgttgagaaatcaagatataataattcatccaatactagtaatgatataaaaaatggctatcatgcttcaaaatactataaccacaaaggccaggaagggcggtctgtcatagctaagaaagttccagtttcggttctaacgcagcaaaggagcacgtcagtttacctaaggataatgcaagtcggagaaggaacatatggtaaagtttacaaggcaaaaaacacgaatacagagaagttagtagcgctgaaaaaattgagattacaaggagagagggaaggctttcccataacctctatacgagagattaaactactacaaagttttgatcatccaaatgtctctactataaaagaaataatggtcgaatctcaaaaaactgtatatatgatatttgaatatgctgacaacgacttaagtggattactattaaataaggaagtccaaatttctcactcacaatgcaaacatctcttcaaacaattgctattaggaatggaatatttacatgataataagattttacaccgtgatgttaaaggctcaaacatcttaatcgataaccaggggaatctaaaaataacagattttggactagcgaggaaaatgaattcgcgagctgattacactaaccgtgtcattacgttgtggtacagaccaccagaactattgttgggaactacaaattatggaacagaagttgacatgtggggttgtggctgcctcctggtggaattattcaataaaactgcaattttccagggctctaatgaattagagcaaatagaatcaattttcaagattatgggaactcctacaataaatagctggccaacgctttacgatatgccgtggttttttatgattatgccacagcaaactaccaagtatgtcaacaatttttctgagaaatttaaaagcgttttaccatcttcaaaatgtctgcaattggcgattaatttgttatgttatgatcagacgaaaaggtttagtgcaaccgaagctttacaaagcgactatttcaaggaagaaccaaagcctgaacctttagttcttgatggattggtaagttgccacgaatatgaagtcaaattggcaagaaaacaaaagcgtcctaacatactatccaccaacacaaacaataagggtaatggtaatagtaataataataataataataataatgacgatgatgataaataa

SEQ ID NO: 181 - DNA, 2148 bp, Saccharomyces cerevisiae
atgggatatcaattgagtgcaacacttaaagggcacgatcaggacgtcagggatgtggtagctgtggatgattcaaaggttgctagtgtttcgagggatggaacagttcgcttgtggtctaaagatgaccaatggctcggtacggtagtttatacaggacaaggatttttaaatagtgtgtgttatgattccgagaaggagttgttgctttttggaggtaaggatactatgatcaacggtgtgcccttgtttgccacctcaggagaggatcccttgtataccttgattggccaccaagggaacgtttgtagtttatccttccaagatggtgttgtgataagcggtagttgggacaaaaccgcaaaggtttggaaagaaggatcgttggtttacaatttgcaagcacacaatgcctctgtatgggatgccaaagtcgtatccttctcagaaaataaattcttgacagcttccgccgataagacgattaaactgtggcaaaacgataaagtgatcaaaactttcagtgggattcacaatgatgtagtaagacatttagctgtggtggatgatggccattttatcagttgttcaaacgatggcctcatcaagttggttgatatgcatacaggtgacgtactaagaacatatgaaggccatgaaagctttgtttattgcattaaactgctgcccaatggagatattgtaagctgcggcgaagaccgaacagtccgtatatggtcaaaggaaaatggttcgttaaagcaggtaattacacttcctgccatctctatttggtccgttgattgtatgtctaatggtgatattattgtgggaagtagtgataatcttgtgagaatattttcgcaagagaagtcaagatgggcttccgaggatgaaataaatgaactatctacgcaagtggaaaaatccactataagttccaagacaatagaatttgatgagtctaaactatcaccttatgaaattttacaatcgccggggcgcaaagagggtcaaattgtggtagtcaaatcacctcaaggtactatagaagctcatcaattttcaaattcttcatggaaaaaggttggtgatgttgtcggtgcaggtgctactggaaacgacaaaaaaatagaattcgagggtaaaacgtatgactacgtatttgatgtggatattgaagatggaaaacctcccttgaaattgccgattaatgtcagcgacaacccatatacagcagccgacaattttttggctcgttatgaactgccaatgagttatagggatcaagtagtacagttcatactgaagaacactaatggcatatcgctagaccagcccaacgataatgcatcatcttctgcagtatcaccttcaaaaacgagcgtaatgaaggttcttcctgtcaagcagtatcttatcatggaaaattataacccagatactattttcaatggaatagtaaaaataaattcaaatgagaagacctttgatgatgaaattctggcgcaaattggtggcgcactacatgacatagatgaaagctgggagttgctactttcatttgctaacactataagatctaactgggaaattaagactcctgcatacgacatagtacgattgattgtaaaaaaattaccttattcttcagatataaaagattacatcgaagaaggcttgggcaataagaatatcactctaaccatgttaactgtgcgtattttggtaaattgcttcaataatgaaaactggggtgtgaaattactggaatcaaatcaagtttataaatcaatttttgagactatagatactgaattttctcaggcatctgctaaacagtcacagaatttggctattgcagtttccacactaattttcaactattcagctctcgtcacgaaagggaactctgatttagaacttttgcctatcgtagctgacgccatcaacaccaagtatgggccattggaagagtatcaagaatgcgaagaagccgcatataggctaaccgtagcttacggaaatttggctactgttgaacctacattaagacaatttgccaactccgtaacttggcttgcaaacatcaaaaggagctatgggaacgtgccaaggtttaaggatattttcgacgatctctcctaa

SEQ ID NO: 182 - DNA, 1593 bp, Saccharomyces cerevisiae
atgacaagaaagcaggctattgactatgcaatcaaacaagttccgcaaatattgcctttggaagagtctgatgtaaaagcgctgtgcgaacaagtactgagtacatcatctgatgatccagaacaaattgcttcaaaatttttggagttcctaggtcatgaagatctatcgtttgaatttgtaatgaagttcaatgaattattgaatcagaatgataaaaaagaggaaaaaaaaacgaaaaatgtgcatcttgaacatactgcccccacttcatggaaaaatgaatcaaaacagcccactaataactatatcaataagaagggagatgaaaaaccaaaaaaattaaaggatgagaagaaaagctcgacaacgaggcctaccgttcaaccatcaaatcaatctacgcaatcaaaccccataaaggagaaaaaagagcacagatcaaaggggaaattacaatcgttacaagaaatagacgaagcaatcaagatgctagagcttcgagatagcggttcttctaaaaattgtaattgtcaaggtacaaggcatccagttttcgacatcgcgccaaactgtcttcactgtggtaaagtggtttgtgttatagagggattgaataaaggtaaatgcggtcattgccatgaacagttgatttctgataacgaaagaacccaaatggtagaaattttaaatcaggaaaaaaacgaactaaacggttcatcatcgtccttatccaatgcgtcaaatggagctaatgtcccaaagaagaagactaaaacctacaaaataacttccggtatgggtaaaaatttatttgctgaacaggacaaattatttgatttcatcgaaaggaaaagagaaagagaaagaaagcgtaatgaagtgttgaaattgcaggaggaaaaagaagagagcgaagcaaaggaaagacaagcgagcgagcatgaccataaagctgaggaaaacccggagttgttagcagcacaagaacgattagatagacttttatatttccaagacacttcggcagaaagaacaaaaattattgataatgccagcgattttgacatgaatcaggaggtgggtttatggggcagcgcaagagaaagagctcttgcgttgaagaagcaacaacgtaatttgagaaagtgggaaaaagtagaaaaggaacggaatggcaggcgcgagaagtacgttgttagcatgaatataggttcaaacggtaaagttactatgacagaagtccctaaggatacagagaacgtaattgcaggttcagatgatgacatcagtgatataagtgacgaagaagatatcagcgaccttaaacatatacacgctctgaaaagtgaaataaatacaacgaaatccttggagaatttgcatttacagtccaaagcatgggattacgaacgtgataaaaaacaattcgataggcctacgtatgtgaaaaaaaattcagatactgcacaacagaatagaaaaacggaagaaaaggctcacgacatgcaagcttatgatttaaagtcaagagtacaagtggaccaaaatgcagacgcatcagtagaacagaatattcttgctgtactttga

SEQ ID NO: 183 - DNA, 3474 bp, Saccharomyces cerevisiae
atgagtgaaaaaaaagtacatcttcgtttgcggaaggaacttagcgtacccattgcggtggttgaaaacgaatccctggcgcagttgtcttatgaagaggaaagccaggcctctctaatggacatttccatggagcagcaacagttaaggttacattcgcactttgataattccaaggttttcacagaaaataacagatacatagtaaaaacccttcaaacagactacagtagcggattcagtaacgatgacgaattgaatggatatatcgatatgcaaattggatatggactagtcaatgaccacaagaaggtttacatctggaatattcactccactcaaaaggatacgccctatataactgtaccatttcgttctgatgataatgatgaaatagcagtcgcgcccaggtgcatactaactttccctgctacaatggatgagtctccattagcactgaatcctaatgatcaggacgaaaccggagggcttatcattatcaaaggtagcaaagcgatatattatgaggatatcaactccataaataatttgaactttaagttatctgaaaaattctctcacgagttagaattacccatcaactcttcgggcggagagaagtgtgacctaatgttaaactgcgaacctgctggtatagtgctttctacaaatatgggcagaatattttttataaccattagaaattccatgggtaaacctcaactgaagttaggcaaactattaaataaacctttcaaattgggtatctggtccaaaattttcaatacaaacagttcagttgtctcattacgaaatggacctatcctcggtaaggggacaaggttagtatatattactactaacaaaggaatttttcaaacatggcagttgtctgccacgaattcccatccaacaaaattgattgatgtaaacatttacgaagccattttagagtcgctccaggatttgtatccatttgctcatggtacgttgaagatttgggactctcatccattacaagatgaaagttcacaacttttcctgtcatctatttacgacagttcatgcaatgaaacatattatattctttccacgattatcttcgattcttcttctaatagttttactattttttccacttatagattgaacacttttatggagtcaataaccgacacaaagtttaagcctaagatatttatacctcagatggagaatgctaacgataccaatgaggtaacttccattttggtaatgttccccaatgctgttgtcattactcaggtgaactcaaaattggactctagctattcaatgagaagaaagtgggaagatattgttagccttagaaatgacattgatataattggttctggttacgactcaaaatctttgtatgttttaacgaaacaaatgggggtgctacagttttttgtgaaagagaatgaggaaacaaattctaaaccggaagttgggtttgtcaaatctcatgttgatcaagctgtttatttctccaaaataaatgcaaatcccatagatttcaatttacctccagaaatttcactagatcaagaatctatagagcatgatttaaaattaaccagcgaggagatttttcattccaacggcaagtacataccccctatgctaaatacgttggggcaacatctctctgtccgtaaagaattttttcaaaatttcctgacatttgttgctaagaacttcaactataaaatatcacccgaactgaaattggatctgatcgaaaaatttgagatcttgaattgctgtattaagttcaatagtatcatcagacaatctgatgtattgaacgatatatgggagaagactttatcaaactataatctgacacaaaatgaacacttaactactaaaacagttgtaatcaacagtcccgacgtatttccagtaatctttaaacaatttttaaaccatgttgtgtttgttttgttcccatcacaaaatcaaaatttcaaactaaatgttaccaatttaatcaacttatgcttttatgatggaattcttgaagaaggtgaaaaaactataaggtatgaacttttggagttagacccgatggaggttgatacatccaagctaccatggttcataaactttgattacttaaactgtatcaatcaatgtttttttgattttacatttgcgtgtgaggaggaaggaagtctggattcttataaagagggcttgttaaagattgtgaaaattttatattatcagttcaaccaatttaaaatatggatcaacacgcaacccgttaaatctgtgaacgccaatgataattttataaatatcaacaatctttatgatgataaccacctggattggaaccacgtcctttgtaaggtaaatctaaaagaacagtgtattcaaatagcagaattttacaaagatttatctggattggtgcaaacattacaaactttagatcaaaatgactcaacaaccgtatcgctatacgaaacattcttcaacgaattccctaaagagtttagctttacattatttgaatatttaatcaagcataaaaaattgaacgacctcatcttcaggttcccacaacaacacgatgttttaatacagtttttccaagagtccgctccaaagtacggtcatgtagcatggatccaacagattctggatgggtcgtacgcagatgccatgaatactttaaaaaacattactgttgatgattcaaagaagggcgaaagcttgagcgaatgtgaattgcacttgaatgttgcaaaattaagctcgttgctagttgaaaaggataatttggacattaatactttgagaaagatccaatataatttagatacaattgatgctgagaaaaatatttcaaacaaattgaaaaagggcgaagttcagatatgtaaacgcttcaagaatggctctatcagggaagttttcaacatattagtggaagagcttaaatcaacaacagtagtcaatctatcggatctcgttgagttgtattctatgctcgacgatgaagagagcttattcataccgctaaggcttctttctgttgatggaaacttactgaattttgaagttaaaaagttcttgaatgctttggtatggagaagaatcgttctactaaatgctagtaatgaaggagataaactgcttcagcatatagtcaaacgtgtttttgatgaagaactacccaaaaataatgatttccccttgcccagtgtggatcttttatgtgacaaatcgttactgacgccagaatacataagcgaaacatatggcaggtttcctattgatcagaatgccatacgtgaagagatatatgaagaaatatctcaagtggaaacgttgaactcagacaactcactcgagataaagttgcactcgactattggttctgtagcgaaagaaaaaaactataccatcaactatgaaaccaacactgtagaatactaa

SEQ ID NO: 184 - DNA, 1806 bp, Saccharomyces cerevisiae
atgctcaaccatcccagccaaggctcagatgatgctcaggacgaaaagcaaggcgacttcccggtcatcgaagaggagaagacccaggctgtaacgctgaaggattcgtatgttagcgacgacgtcgccaactccacggaacgctacaacttgtccccttctccggaggacgaagacttcgaaggccccactgaagaagaaatgcagactttaaggcacgttggtggtaaaattcctatgaggtgttggttaattgctattgtagagctttccgagagattctcctactacgggctttccgcaccattccaaaactacatggaatatggacctaatgactccccaaagggtgttctgagcttgaacagtcagggtgccactgggttgtcgtattttttccagttttggtgttacgttacaccagttttcggtggttacgttgcggacaccttctggggtaaatataatacaatttgttgcggtaccgctatttacattgccggtattttcattctatttatcacttcgattccctccgttggtaacagagacagtgctattggtgggttcattgctgccattattctgatcggtattgccactggtatgattaaagctaacctttccgtgttgattgccgaccagcttcctaagcggaaaccctccatcaaagttttaaaatcgggcgaaagagtcattgtcgattcaaatattactttacaaaacgtttttatgttcttctatttcatgatcaatgtcggttctctatcattaatggccactactgaattggaatatcataaggggttctgggcggcctatctattgcccttctgcttcttttggatcgctgttgtcactttgatttttggtaaaaagcaatacattcaaagaccaatcggagataaagtcatcgctaaaagttttaaagtttgttggattttaactaagaataagttcgacttcaacgctgctaaaccttctgttcatccagaaaagaactatccatggaatgacaaatttgttgatgaaattaagagagctttggcggcttgtaaagtctttatattctacccaatttattggacccaatacggtaccatgatttccagtttcatcactcaggccagtatgatggaattacatggaattcccaacgatttcttacaagcattcgattccattgcgttgatcattttcatcccaatttttgaaaaattcgtatatcctttcattagaagatacactccactaaaaccaattacaaaaattttcttcggtttcatgtttggatcttttgccatgacatgggctgctgttctacaaagtttcgtttacaaggctggtccatggtataatgaacctctgggtcacaacaccccaaatcatgtccacgtttgctggcaaatacccgcatatgtcttgatttctttttcagagatctttgcctctatcactgggttggaatacgcttattccaaagccccagcttccatgaaatcgtttattatgtccattttcttattgactaacgcctttggttctgcaatcggttgtgcattgtccccagtgaccgttgatcctaaatttacatggttattcactggtttggctgttgcctgctttatttctggttgtttgttctggttgtgcttcaggaagtataatgatacagaggaagaaatgaacgctatggactacgaagaagaagacgaatttgatctcaatccaatttccgcacctaaagctaacgatattgaaatattagaaccaatggaaagtctaagatccaccaccaaatattag

SEQ ID NO: 185 - DNA, 723 bp, Saccharomyces cerevisiae
atgtggtcatcactttttggttggacatcaagtaatgccaagaataaagagtcaccaacaaaggccatagtgcggttgagggagcatatcaaccttctatccaaaaagcaatcgcatttacgtactcaaattacaaatcaagagaatgaagctagaatctttttgacgaagggcaataaagtaatggcgaagaatgcacttaaaaagaagaagacaatcgaacaacttttaagtaaggtagaaggcacaatggagtctatggaacagcagctattctctatagaaagtgcaaacttaaatctagagacaatgagggctatgcaggaaggtgctaaggcaatgaaaactattcacagtggccttgacatagataaagtggatgaaactatggacgagataagggagcaagtcgaattaggagatgaaataagcgacgctatatccaggcctttaattactggggcaaacgaggtggatgaagatgagctggacgaggaattggacatgctggctcaagaaaatgctaaccaagaaacgtccaagatcgttaataataatgttaatgcggcgcctatctcagagaacaaagtctcactacctagtgttccaagtaataaaattaaacaaagtgagaactctgtgaaggacggggaagaggaagaggatgaagaagatgaagatgaaaaagcattaagagaactacaagcagaaatggggctttga

SEQ ID NO: 186 - DNA, 1257 bp, Saccharomyces cerevisiae
atgtctgccactctgttcaataacatcgaattgctgccccctgatgccctttttggtattaagcaaaggtacgggcaagatcaacgtgctaccaaggtcgacttgggtatcggggcctacagagacgacaacggtaaaccatgggtcttgccaagtgttaaagccgccgaaaagctaattcataacgacagctcctacaaccatgaatacctcggtattaccggtctgccaagtttgacatctaacgccgccaagatcatcttcggtacgcaatccgatgcctttcaggaagacagagtaatctcagtacaatcactgtctggtacgggtgctcttcatatatctgcgaagtttttttcaaaattcttcccagataaactggtctatttgtctaagcctacttgggccaaccacatggccatttttgagaatcaaggcttgaaaacggcgacttacccttactgggccaacgaaactaagtctttggacctaaacggctttctaaatgctattcaaaaagctccagagggctccattttcgttctgcactcttgcgcccataacccaactggtctggaccctactagtgaacaatgggttcaaatcgttgatgctatcgcctcaaagaaccacatcgccttatttgacaccgcctaccaagggtttgccactggagatttggacaaggatgcctatgctgtgcgtctaggtgtggagaagctttcaacggtctctcccgtctttgtctgtcagtcctttgccaagaacgccggtatgtacggtgagcgtgtaggttgtttccatctagcacttacaaaacaagctcaaaacaaaactataaagcctgctgttacatctcaattggccaaaatcattcgtagtgaagtgtccaacccacccgcctacggcgctaagattgtcgctaaactgttggaaacgccagaattaacggaacagtggcacaaggatatggttaccatgtcctccagaattacgaaaatgaggcacgcattaagagaccatttagtcaagttgggcactcctggcaactgggatcatatagtaaatcaatgcgggatgttctcctttacaggattgactcctcaaatggttaaacgacttgaagaaacccacgcagtttacttggttgcctcaggtagagcttctattgctggattgaatcaaggaaacgtggaatacgtggctaaagccattgatgaagtggtgcgcttctatactattgaagctaaattgtaa

SEQ ID NO: 187 - DNA, 1188 bp, Saccharomyces cerevisiae
atgtctgctacatcttcaagtggtgacgtgaaaaaatttcaagctgtaccaaaaccaacgagcaatgcatcaccaccaccagcatcgagtgggtttaacgcaaggaccctatggccggatttgatagaaacaccagaaaaccagtgggtttttgagtgcaaagatattattgaaaagataggcactaatggaccgatagcagtggaaataaaaaaaaatatggaaaaatgcttaatgtacttttacacacttaaaaaaaaattaaatctttttgatcacacctatacggcatcatgcattctattttacaggtattggttcatatatggaatacctacagccataactgaatgtattcatatatcacagggaattctggtcaccgcttgcaaaacaatggagaataataggcctatagaggcctatattaaggcgacttgtgagtttttgatgcaaaatataccgtctttaaaaagtcgaacaaatattgataaacttaaatgggaatttagagataagttagtgacgaatgaaaaaaaaatactatgtttatttggctttgacttaaatatcagtaatccaaaagaattgattgaagaagtgttcagtggctattatagattcaacagggatcacaatctgccagaaaactttaaaaaagcttttccaaaaattcttcaagaatcaagaaattttatggtccaagctgtcacccagccagtttctttattgtgcgacggttatacgttcattgtactatctctcatatactgtggtttagagtataaaaagcttgtagacaaagatttcagatatccgaaaaacttcttcaaggatagattccctatcgaagtaacgccggaaaattttgccaatatctttaccgattacaagttactggaagagaattttttcaacttaaaaagtaataaaggtgctaaattacaaattgattcaagcatgattgattcggtgattgacgaaagtggcgatgtggaaaatgaagtttcagaaatctctgatccgtttaactacgagttgattaaatccggtgaggtgaaggaagagtttttgaatcatattgaaacaagggttaaagatttactagataaggctaaacaagaaagtatgaagagaaaggctaaagatcctataagaaccccagatgccaaaaaacctaaaatataa

SEQ ID NO: 188 - DNA, 1971 bp, Saccharomyces cerevisiae
atgaaagtgctatgtgtcgcagagaaaaattctatagcgaaggcagtttcacagatcctaggaggaggcagatcaacttcaagggattccggctacatgtatgtaaagaactatgatttcatgtttagtgggttcccgtttgccagaaatggggctaactgcgaagttaccatgactagtgttgcagggcacctaacaggcattgatttcagccatgattcgcatgggtggggaaaatgcgccatccaagagttatttgatgcgccactgaacgagattatgaataacaaccaaaaaaagatagcaagcaacatcaagcgagaagcgaggaatgcagactatctgatgatatggacagattgcgaccgggaaggagagtacatcggttgggagatatggcaggaggccaagagaggcaacaggctcatacaaaatgatcaagtataccgggcagtcttttcgcatctcgaaagacaacacatattaaatgcagcacgaaacccaagtcgattggatatgaagagtgtgcacgctgtaggcacgcggattgaaatcgatcttcgagcaggtgttacattcaccagactcttaacagaaacgctacgaaataaactgagaaaccaagccaccatgaccaaggatggtgcaaaacaccgcggtggtaacaagaacgactcacaagtcgtatcgtatggtacatgccagtttccaacgctcggctttgtagtagacaggtttgaaagaatacgaaattttgttcccgaagagttctggtatatccaattggtagtcgaaaacaaagacaacggcggaacaacaacgttccagtgggacaggggccacttgttcgaccggctgagcgtgttaacgttttacgagacatgcatcgaaaccgccggcaatgttgctcaagtagtagacttgaaatcaaagccaacaacgaaatacagacctttacctctgaccacagtggagctacaaaaaaactgcgcccggtacctgcgtctgaacgccaaacaatcactagacgcagcagaaaagctataccaaaaggggttcatatcgtatccaagaacagagactgatactttcccacacgcaatggacctaaaatccttggtcgaaaagcaagctcaattggaccaactcgctgcaggcggcagaaccgcctgggcatcgtacgcggcatcgctgctccaacccgaaaacacaagtaacaataacaagttcaagtttccacgaagcggctcccatgacgacaaagcgcatccaccaatccaccccatcgtaagtctggggcctgaagcaaatgtttcgccagtggaaagaagagtatacgagtacgtggccaggcactttttggcatgctgctcagaggacgccaagggccaatcgatgacccttgtgttggactgggccgttgaacgtttctcagcttcaggtctcgtagtcctagagagaaatttcctcgatgtttacccttgggcccgatgggaaaccaccaagcagttaccgcggcttgaaatgaatgccctcgtagacatcgcgaaggccgaaatgaaggcgggcactacggcgccgcccaagccgatgactgagagtgaactcattctcctcatggatacaaacggcattggcacagacgccaccattgcggagcacatagacaagatccaagtacgtaattacgttaggagcgagaaagtaggcaaggaaacctacttacaacccacgaccctgggtgtctcactagtgcacggcttcgaggccatcggcctcgaagactcctttgcaaagcccttccagcgcagagaaatggagcaagacctcaagaaaatctgcgaaggtcatgcctccaagactgatgttgtaaaggacatagtcgagaagtataggaagtactggcacaagacgaatgcctgcaagaatactctcttgcaagtttatgaccgtgtcaaggcatccatgtaa

SEQ ID NO: 189 - DNA, 2628 bp, Saccharomyces cerevisiae
atgtcactgaacaacataacattctgtgtctcacaggatctggatgttcccctgaaagtgaaaatcaagtcattggaaggacataagccactgttgaagccatctcaaaaaatcctgaaccctgaattaatgctgatagggtcaaatgtattcccttctagtgatctaattgtatccttacaggtatttgataaagagagaaacagaaatttaactcttccgatatacacgccgtatattcctttcagaaactctagaacctgggattattggttgactttgcccatacgtatcaaacagcttacctttagcagtcatttacgcattattttgtgggaatacaatggatccaagcaaattccctttttcaatctagaaacgagcatctttaacttaaaagactgtactttaaaaagagggtttgaatccttaaaatttcgttatgatgtgattgaccactgtgaagtggtcactgataacaaagatcaagaaaatttgaataaatattttcagggtgaattcacgagactcccttggcttgacgaaatcactataagcaaattaaggaaacaacgggaaaataggacttggcctcagggcacctttgtcttaaacttagaatttccaatgttagagcttcctgttgtgttcatcgaaagggaaattatgaatactcaaatgaatattccaactttgaagaacaatcctggactaagcacagacttacgagaaccgaataggaatgatcctcaaatcaaaatttctttgggggacaaatatcactccacattgaagttttacgatcctgaccaaccaaacaatgatccaatagaggaaaagtataggagattggaaagagcatctaaaaatgcaaacttggacaagcaagtgaagccggacattaaaaagagggactacttgaataaaatcatcaactacccccctggcactaaattaacagcacatgaaaagggttcaatatggaaatataggtattatttgatgaacaataaaaaagctcttacaaagttattacagagtacaaatttgagagaagaatcggaaagagtagaggttttagaactaatggactcatgggcagaaattgatatagacgacgcattagaactattaggctcaacttttaaaaacctttcagtgagatcgtacgccgtaaataggctgaagaaggcatcagacaaggagctggaattgtacttattacagttggtagaagctgtctgttttgagaacctgtccactttttccgacaaatctaacagtgaattcactattgtggatgccgtatcttcgcaaaagctttcgggtgattctatgttactatctacatcgcatgccaaccaaaaattattaaagtccatctcgagtgaatcggaaacttccgggacagaatcgctaccaatcgtcatttctcccttggctgagtttttgattaggagagcactggtcaacccaaggttaggaagttttttctattggtatttaaagtccgagtctgaggacaagccatatttagaccagattttaagttccttttggagtagactggataaaaaatctcggaatatactgaacgatcaagtcaggctaataaatgtgctcagggaatgttgtgaaacaattaagaggctgaaagatactacagccaaaaagatggaattactagtgcatttattggagacgaaagtcaggcctcttgtcaaagtacgaccaattgctttaccactagaccctgatgtgttgatatgcgacgtttgtcctgaaacttcgaaggtatttaaaagctctttatcgccactaaaaataacattcaaaacgaccttaaaccaaccatatcacttaatgtttaaggttggcgatgatttgagacaagatcaactggtagtacagatcataagtttaatgaatgagttattaaaaaatgaaaacgtggacttgaaattgacaccatacaaaattttggcaacaggaccacaagaaggtgccattgaatttatccctaatgacacattagctagcatactaagcaaatatcacggtattcttggctaccttaaactccactatcctgatgagaacgcgacattaggcgtacaaggctgggttttagataattttgtcaaatcttgtgctggctattgtgttattacatacatcttaggtgtcggcgataggcatttagacaacttactagtcacgccagatgggcacttttttcacgcagactttggttatatcttgggtcaggaccccaaaccttttccgccattaatgaaattgccccctcaaattatagaggcgtttgggggtgcagaatcatcaaattatgataaatttcgcagctactgttttgtggcatattcgattttaagaaggaatgcaggcttaatcttaaacctgtttgaattaatgaagacttcgaacatacctgatatcagaatagatcccaatggtgctatattacgtgtaagagagagatttaatttgaatatgtccgaagaagatgccacagtgcattttcagaatctaatcaatgatagtgtaaatgctttgctgcctatcgtgattgatcatttacataatctggcacaatactggcggacctga

SEQ ID NO: 190 - DNA, 315 bp, Saccharomyces cerevisiae
atgagacactgcattatctttatagtgtgtatcagcattgttgaaattagaaccgttcatattgaattcatcaaagagatcgttgtcatattccgcatagttgaccactttagcccttttatgctgccttgtttgttgagccattgtaaagatggggataccatcatcttcgtttgtcaatctgttatgaaagttagaaatataagcttgtggaataagctggttttggtgcgacattgtgtgctgctatgtgcctttcttctttcttttttcaatgtacttcattcaataatctctatttgtcgtattttctaa

SEQ ID NO: 191 - DNA, 2016 bp, Saccharomyces cerevisiae
atgtcttcagttgatgtactgttaacagtaggtaagttggatgcctcattggcgttactgactactcaggatcatcatgttattgagtttcctacagtattattaccagaaaatgttaaagctggatctatcataaaaatgcaagtttcacaaaatttagaggaggaaaaaaaacaaaggaatcattttaagagtatacaagccaaaattttggagaagtatggtacccataaaccggagagcccagttttgaaaattgttaacgttacgcaaacgagctgtgttctagcatgggatccattgaaacttggctcagcaaaattgaaatcactgatcctttataggaagggaatacgttcaatggtaattccaaatccattcaaagtgactaccacgaaaatatccggtctttccgttgatacgccatacgaatttcaattgaaactgataaccacgtcaggaacattatggtctgaaaaggttatattgcgtacacataagatgactgacatgtctggtatcactgtatgtttgggtccattggatccattgaaagaaatttcagacttacagatatcccaatgtttgtctcacatcggggcgagacctttacaacgtcatgttgcgatagatactacgcattttgtctgtaacgatctagacaatgaagaaagcaatgaagagcttataagggcaaaacataacaacataccaattgtcagaccggaatgggtgagagcttgtgaggttgagaaaagaatcgttggtgttagaggattttacttagatgcagatcaaagtatactgaaaaactacacattcccaccagttaatgaggaagaactttcgtactcaaaggagaatgagccggtagccgaagtagcggatgaaaataagatgcccgaggacacaacagatgtcgaacaggttgcatcacctaatgacaatgagagtaatccttcagaagctaaggaacaaggagaaaagagtggacatgaaactgccccagtaagtcctgtagaagatccattgcatgcttcgacggctttggagaatgaaaccaccatcgaaaccgtcaacccctccgtaagaagtttgaaaagcgaacctgttggtactcccaatatagaggaaaacaaagcggactcttccgcagaagccgtggtagaagaaccgaatgaagctgtggctgaaagttctccaaatgaagaagcaacgggacagaaaagtgaggataccgatacacattctaacgaacaagctgataatggatttgtacagactgaagaagtagctgaaaacaacataaccacagaaagtgcaggggaaaataacgaacctgcagatgatgcagcaatggaatttggacgtccagaagctgaaattgaaactccagaagtaaatgagtctatagaagatgccaatgaacctgcggaggattccaatgaacctgtggaggattccaacaaacctgtgaaggattccaacaaacctgtggaggattccaacaaacctgtggaggattccaacaaacctgtggaggattccaacaaacctgtggaggatgccaatgaacctgtggaagataccagtgaacctgtggaggatgccggtgaacccgtacaagaaaccaacgagtttactaccgacattgcctctccaagacatcaagaagaagatatagaacttgaagccgaacctaaagatgctaccgaaagtgttgcagtcgagccatccaatgaagatgtaaaaccagaagaaaaaggttcagaggcagaagacgatatcaacaacgtttccaaggaggctgcctctggtgagagtactacccaccaaaaaactgaggcctctgcttctcttgaaagcagtgccgtcacggaagaacaagagacaacggaagccgaagtaaatacagatgacgttttgtccactaaagaagctaaaaaaaatactggcaacagcaacagtaataagaagaagaataagaagaataagaagaaagggaaaaagaaatga

SEQ ID NO: 192 - DNA, 2454 bp, Saccharomyces cerevisiae
atggcaggtgctccagctcctcctcctccaccgcctccaccagcattaggtggtagtgctcctaaaccggcaaaatctgtgatgcagggccgtgacgccctgttaggtgacattagaaagggaatgaaactaaagaaagctgagaccaatgatagaagtgccccaatcgtgggaggtggtgtcgtttcatcggcctcaggaagttctggtaccgtcagtagtaaaggaccctctatgtcagctcctccaatccctgggatgggcgctccgcagttgggcgatattttagctggaggtataccgaagttaaaacatatcaataacaatgcaagcactaaaccctctccatcggcgagcgcaccgccaattccgggcgcggtgccatctgttgcggctccccccattcctaatgcaccattatcaccagcacccgctgttcctagtattccctcgtcttctgcacctccaattcctgatattccatcttctgcggcccctcctattcccattgtaccatcatctccagcaccaccgcttccattatcaggtgcctctgctccaaaggtaccgcaaaatagaccgcatatgccttcagtaaggccagcacacagaagccaccaaagaaaaagctctaatatttctcttcctagtgtgtcagcaccccctttgccttcagcgtctctgccaacacatgttagcaaccctccacaagctccaccccctcccccaactccaacaattggcctggactcaaaaaacattaaacctacagataacgcagtctcaccaccatcttctgaagttcctgcaggcgggttgccatttttggcagaaattaatgccaggaggtctgaacgaggagccgtcgagggcgtttcttccacaaagattcaaaccgaaaatcataaatctccttcacaaccaccacttccatcatctgctccgcccattcctacatcacatgctccgccattaccgcctactgcaccacctccaccatcgttaccaaatgtgacctctgcacctaaaaaagcaacttcagctccagctccgcctccaccaccactaccagcagccatgagctctgcgtcgactaacagcgtaaaagcaaccccggttcccccaactttagcaccaccgttaccaaatactacatcagttcctcctaacaaagcatcgtcaatgcccgcgccaccacctcctcctcccccaccacctggtgctttttcaacatcctctgccttgtcagcatcatctattcctttagcaccactaccacctcctcctcctccttcggttgcaacgtcagtaccatcggctcctcctcctcctccaacactcacgacaaacaaaccctcagcgtccagtaagcaatccaaaatatcttcatccagttcttcctcggccgtaacacctgggggacccttaccatttcttgctgagatccaaaagaagagagatgatagattcgtggttggtggtgatactggatacactacccaagataaacaagaggatgtaataggttcatctaaggatgataacgtaagaccttctcctatttctccatctataaacccgcctaaacagagtagccaaaatggtatgtcgtttctggatgaaattgaatccaaattacataagcaaacatcatcgaatgcttttaacgcaccacccccccatacggatgctatggcgcctcctttgcctcctagtgcacctcctcctcctataacatcgctaccgactccaactgcatcaggtgatgaccacaccaatgataagagtgagactgtgttagggatgaaaaaagcaaaggcccctgcactgcctggacacgttccacctccaccagttccaccagttttatcagatgatagcaagaataatgtccccgccgcttctttattacacgatgttcttccctcctcgaatctcgaaaaacctccttctcctcctgttgcagctgctccgccattgccaacatttagtgcaccaagtctgcctcagcaatccgtgtctacatcaataccatcacctcctcctgttgcgcctacactttccgttcgtaccgaaactgaatcaatttcgaagaaccccactaagtcgccacccccaccaccaagtccatctacgatggataccggtacctctaatagccccagtaaaaaccttaaacaacggttattttctacaggtggatcgacgctgcaacacaagcataatacgcatacaaatcaaccagatgttgatgtaggtaggtatacgatagggggatcgaattctatcgttggggccaaatctggaaatgaaagaattgttatagatgattcaagatttaaatggactaatgtgtcacaaatgcctaagccacgtccctttcaaaataaaacaaaactgtatccaagtggaaagggtagtagtgtgccattggacttaacattatttacgtga

SEQ ID NO: 193 - DNA, 858 bp, Saccharomyces cerevisiae
atgttttttgagtccaggccaattgttggagttgggggagggggtggagcttgtggagggttgctaacatgtgttggcagagacgctgaaggcaaagggggtgctgacacactaggaagagaaatattagagctttttctttggtggcttctgtgtgctggccttactgaaggcatatgcggtctattttgcggtacctttggagcagaggcacctgataatggaagcggtggtgctggagatgatggtacaatgggaataggaggggccgcagaagatggaatatcaggaattggaggtgcagaagacgagggaatactaggaacagcgggtgctggtgataatggtgcattaggaatggggggagccgcaacagatggcaccgcgcccggaattggcggtgcgctcgccgatggagagggtttagtgcttgcattgttattgatatgttttaacttcggtatacctccagctaaaatatcgcccaactgcggagcgcccatcccagggattggaggagctgacatagagggtcctttactactgacggtaccagaacttcctgaggccgatgaaacgacaccacctcccacgattggggcacttctatcattggtctcagctttctttagtttcattccctttctaatgtcacctaacagggcgtcacggccctgcatcacagattttgccggtttaggagcactaccacctaatgctggtggaggcggtggaggaggaggagctggagcacctgccatttcaacaattcgttatatatatggtaggcttcttcaacaaacggttgtgcgctttttagtcgtatgctttgtctcatatcaaaaactttcttcataa

SEQ ID NO: 194 - DNA, 1683 bp, Saccharomyces cerevisiae
atgtctcaaagaaagctacaacaggatatcgataagcttttgaagaaggtgaaggaaggtattgaagacttcgatgacatatacgaaaaatttcagtcaacagacccctccaattcttcgcatagagaaaaactggaatctgacttaaaaagagaaatcaagaagctgcaaaaacatagggatcaaataaaaacatggctcagcaaagaagatgtaaaggataagcaaagtgttctgatgactaataggaggttgattgaaaatggcatggaaagatttaagtccgtggagaagctaatgaagacaaaacagttttcaaaggaggctttaacaaatccagatataatcaaagaccctaaagaactcaaaaaaagagatcaggtcttatttattcacgattgtttagatgagctacaaaagcaattggaacagtatgaggctcaagaaaatgaagaacaaacagaaagacacgaatttcacatcgccaacctagagaatatcttgaaaaaacttcagaataatgaaatggacccagagcccgtagaagaattccaagacgatataaagtactatgtagaaaataacgatgatcccgatttcattgaatacgatacaatttatgaggacatgggttgcgaaatacaaccttcatcaagtaataatgaggctccaaaagaaggaaacaatcaaacttctttatctagcattcgctcatcaaaaaaacaagaacgttctccaaagaaaaaagctccgcagagggatgtttctatatcagacagggcgacgactccaatcgcgccaggtgtagaatcagcctcgcaatctatatcttctacgccaacacctgtgtcaactgatacgccattgcatacagtgaaagatgattcaataaaattcgataattctacccttggtacaccaactacacatgtgtctatgaaaaagaaggaatcggaaaacgactcggagcaacaactaaatttcccaccggatagaactgatgaaattcgaaaaaccatccagcacgatgtagagacgaatgcagcttttcaaaatcccttattcaacgacgaattaaaatactggttagactcgaaaaggtacttaatgcaacctcttcaagaaatgtcaccaaagatggtatcgcaattagaatcttcgctattgaattgtccagattctctagatgccgattcgccatgtctatacaccaaaccattatctttgcctcacccaacgtcaatcttttttcccaatgaaccaattcgatttgtttatccgtacgatgtacctttaaatttaacgaataatgaaaatgatactgacaataagttcggtaaagatagtaaagcaaaatctaagaaagatgatgatatctattccagaacctcactagccagaatatttatgaaatttgatcttgatactttatttttcatattctatcattaccaaggatcatatgaacagtttttagctgctagagaactttttaaaaatagaaactggctattcaacaaggtagatcgctgctggtattataaagaaatcgaaaagttaccaccaggaatgggcaaatctgaggaggaatcatggagatactttgattataaaaaaagttggttagcgagacgttgcggtaacgacttcgtatataatgaagaagatttcgaaaaactgtaa

SEQ ID NO: 195 - DNA, 1314 bp, Saccharomyces cerevisiae
atgagcacgggagattttttaactaagggaatagaactggttcaaaaggccattgacctggacacagccacgcagtatgaggaggcgtatacagcatactataacggattagattatttgatgttggctttgaaatacgagaaaaaccctaagtcgaaggatttgataagagcgaagtttacagagtatctaaaccgtgcagagcagttgaaaaagcacttggaaagtgaagaggcaaacgcggcaaagaaatctcctagcgctggtagtggttccaacggtggtaataagaaaatctctcaggaagagggtgaggataatggtggagaagacaacaaaaagctaagaggtgccttatcgagtgctattttgtctgagaagcccaatgttaagtgggaagacgtggctggactggaaggagccaaagaggcccttaaagaagccgttattttacctgtcaagttcccacatttgttcaaaggtaaccgtaagcccacttcgggaatcttattgtacggaccaccaggtacaggtaaatcatatttggcaaaagcagtggccaccgaggccaattctacctttttcagtgttagttccagtgatttggtttctaagtggatgggtgaatctgaaaaacttgtcaaacagttatttgcgatggcaagagaaaataaaccttctattatttttatagatgaagtggatgcgctaacaggtaccagaggagaaggagagagcgaagcaagtagaagaattaaaacagaattattggtacaaatgaatggtgttgggaatgactcccaaggtgttttggtccttggcgcaacaaatatcccatggcaactggacagtgccatcaggagaagatttgaaagaagaatatatataccattaccagatttggcggcaagaaccaccatgttcgagattaacgtcggcgacactccatgcgtattgaccaaggaagattatagaactttaggtgcaatgactgaaggttattccgggagtgatattgctgtagtggtgaaagatgcgctaatgcaaccgataagaaaaattcaaagcgcgacacatttcaaagatgtctctacggaggacgacgaaacaagaaaactaacaccatgctctccaggtgatgatggcgccatcgagatgagttggacggatattgaagctgatgaattaaaagaaccagatctcaccataaaggatttcttaaaggctatcaaatcgacgagacctaccgtgaatgaggatgacttgctgaagcaagaacagttcactagagattttggtcaagaaggtaactag

SEQ ID NO: 196 - DNA, 1668 bp, Saccharomyces cerevisiae
atgtataatggcaaggatagagcacaaaactcctatcagccaatgtaccaaaggcctatgcaggtacaaggacaacagcaagctcaatcgttcgttggaaagaaaaacacaatcggaagtgtgcatggaaaagccccgatgctaatggccaataatgatgtttttactattggaccttatagggcaagaaaagatagaatgcgggtatctgtcttagaaaagtacgaagttattggctacattgctgcgggcacatatggtaaagtttacaaagcgaaaagacaaatcaactccggtaccaattccgctaatggttctagtctgaatggtaccaatgcgaaaattccgcagtttgacagcacgcaaccaaaatcaagctcttcaatggacatgcaggcaaatacaaacgcattaagaagaaacttgttaaaggatgaaggagtgacccccggaagaatacgaactacgagggaagatgtatccccgcactataattcccaaaaacaaaccctcattaaaaaaccgctgacggtattttatgccattaaaaagttcaagacagagaaggatggcgtcgaacaattgcattatacgggaatatctcagagtgcctgtagagaaatggcattatgtcgagaattgcacaacaagcatttaaccacattagtggaaatttttttggaaaggaaatgtgtccatatggtatacgaatatgcggagcatgatctgctacaaattatccacttccattcccatcccgaaaaaaggatgataccaccaagaatggttcggtctattatgtggcagcttttagacggcgtatcgtatcttcatcaaaattgggtgcttcatcgagatttgaaacccgcaaatataatggtgaccatagatggatgtgttaaaattggtgatttaggtttggccagaaaatttcataatatgctgcaaaccctctatactggggataaagtggttgtcactatatggtaccgtgcacctgagttgctattgggagcacggcactatacccctgcggttgatttatggtccgttggctgcatttttgcagaactgataggattacagcccatatttaaaggtgaagaagctaaactagactctaaaaagactgttccatttcaagtgaatcaactacagagaattttggaagttcttggcactcccgatcaaaaaatttggccttatttggagaagtatccagaatatgatcaaattacgaagtttccaaagtatagggataaccttgctacatggtatcattccgcgggaggaagggacaagcatgctttaagcttactttaccacttgttaaattatgatccaattaaaagaatagatgcatttaatgcgttggaacataagtacttcacagaaagtgatattcctgttagtgaaaatgtatttgaaggtctaacttacaaatacccggcaagaagaattcacacgaacgataatgacatcatgaatcttggatcaagaacgaaaaacaatacacaagcttcaggaatcaccgcaggtgccgctgcaaatgcgttaggtgggcttggtgttaaccgtagaattctggccgcggcagcagcagccgctgctgcggtgtcaggaaacaatgcatcagatgagccatctcgaaagaaaaacagaagatag

SEQ ID NO: 197 - DNA, 2397 bp, Saccharomyces cerevisiae
atgaaaaaccctagcttcgactgggaaagattgaaagacgtattttatagaagcagagccattggcgaattaaaatggccaactcagtacgaggagttcaaatgtgccttgtcattaactgttatcgcggttgaaattcaagactttatacaagtatacaattattttgggcagctgttaggcaagataaatttgcaaagaattcatgaggatataataaagtttgagttcgataaagacgagaagctgatattggtcacgaaaagctccatcaaaattgtgaaaggttggtcgccactgacgatagaaagtgtcccattacaggacccaaccattgatactatctgggactatcacaatggaatcatgttgttagcaaaatctcgtgatatatacaagttaaacggaaatgaatgggaacttctttacgagaacaaagataaaaaatataatttattgacaaaaaaccactggagttgcaatgatgactccatcattttactggatgttgatcatgtatatcaagtaagcacctcgaacggggcactattgaagttaatcacagattcgtcgtggcacaaggtaacgatctcatctagaggatttatttgcctttataatatgaaggacaataaactgcagattttcagagatccagcaagaatactaatggaacacaacttggattctacaccagatgatatatgttggtgtggcaatgataccgttgcttgttctttcgaggacgaaatcaaattatacggcccagatggtttgtatgtaacattttggtacccttttactgtaactaacctaagggcagaggttgatgggttgaaggttattaccactgaaaagatttactttctatccagggtacaacctcagacttctaatatatttagaattggctcaaccgagcctggtgcaatgcttgttgactcatttagtttactggaggatcatgcacccaaagcaattgaaatattaaagaattttgtcttagagaagggtgtactggactgtattgctgctgcaatagacgaatttgaacctaaactacagaagatgttgttgaacgctgcatcttatggaaaagcttctttgcagtacaagtcatttgacgctagtatatttgtgaatgcgtgtaatacgataaaattattaaactgcttcagaagttttggtatttttttgactgtggaggaatacaggtgtatttctttgaaaggtgtcattgataggctgttgaagtatcacagatattatgagtgcatacagatatgcaaattggccaatgaacgatttctgttaggatacgtgtttactgagtgggcaaaggataaaatcaagggtagtcctgacatggaagatgatgagttactagacaaaatcaaatctcgactaagtgttatagatatgacggatacattacaaatggtagcagttgcgaaagttgcatatcttgaaggaaggttccagttatcccgtaatttagctttattagagaaaaacgaagaagctagaattgagcaactgtataatcttgatgatgatagtatagcgctaaaggagtgtatcaaagtacaaaattacagtctgacaatttcattactgattgcactttccaaaaagctgacaaactctcaactgacgaagcttttgatcattgatatgttcaacaatcccttatatctctattatatgagaatggataaagcatacttgtacgatttttaccgccaaactgaccgatttattgacttggctcacgtattactgcagcagggaaaagaacagcaatctttacacagctttttaccgcaaattaaggacctttatagccaagttcaaaacagtgaagtggtcaacaatacaatagaacaacttcaaagacaggaaaagttgtggatataccaagaatcattaggcaaaagatttgctatatcattcactaatatgactttagatcaaacgctttcgaagttaatagaaacagggcaagataaacaggtgaaagaaatagttaaaaagttcaaaataagcgaaaaaaagctgtatcacctgaaatgtaaaacgttagtcgaagctaagaagtttgacgaactactacagtttgctcagtcgcggaaatcaccaattggttacatgcctttctacacatacttaaaaagcagaggccatatggataaagctagtccttatgttaatatgatacctggcctttcgtatcaggaaaagaaaaagctttatgtggagtgcaggggttttcgtgatgcaattcaactagcaggaaaggaaaaagacattccaggcttaaaggagatatataatattataccacctaatgagccggaattaaaggcattagcaaatgaaactatgagcaggatatag

SEQ ID NO: 198 - DNA, 1134 bp, Saccharomyces cerevisiae
atgcaatcttctcttcccctttgcagagaattttttgaaaaaataactgcatatttagattatcatgatttcaggttaaccattactgccaatcagccttcaattactctcccttactacgtagacgaaaaagcccattccatagaactaattatcttcaaaacaacatttttatctcttttccaagaagcacatacatattttaacaaaactttctctgatcaaagcggcatttctaatgagaatatttattacatgactgttggatttctacttacaacacctgaaaacaaaacggtatataatgtacacgaagatttactcaagagatacttccaggacaacagtgttttagtaataccggatttattggtcaaagaagtaagactgattcaacggcttctttgttcatcaaacaatcgtatcaacaagtcgtcatctctttggatattataccgaaaactattcgttttatctcttgatgctaatacactggttcttccagatattttatttgtatttcattcttctggatcccaacatttctctaattactactgttggaatactgctaggtggttctacgataatttaccttacaataaaagaattgagctcttcaatttgactaagagattttgctttcaaaatgttaaggattgttcctcatggagcgcattagcatatatggtatgccagcaggaggaaaaaaaaaccgataatatacgtgattttcaaagactaacaagctcttttaatgttccgttcaagataaataaagtggatttgaattttcaagtacagcctgcggatgcatttactcaggaattagtaaaatggattgatcgaacgtatgctgcagattggcctccatatctgtgtcttctgcaaattacgaaatttaatatcacccttagaattgaaatggactctgtcttattgacttggagaaatgaaattctgaattttgaagagaacagtggtcatatcaaaatgataaataatactccaatagtacctgagaaattttcaaacgatcttctaacttctgtgaattttgcacattttggctataaaaagcttttcttaaataagtttttggataaaaataagaaagaacaatcagatagttag

SEQ ID NO: 199 - DNA, 537 bp, Saccharomyces cerevisiae
atgcctgcgtatcactcgactttccctgtagatcctaacactgaccgcatggtagggaactttgcgcttctgcccttgaacactaagttcaggggtcctgcataccccagcaattccgattacgatattatcgacgagtgcttggacctatttcgtgccaattccttcttcaagaattttgaaattaaatcacccgccgacagagtgctcatctacggaattctctttatcaatgattgcctagcccatttgaaaataacaacgtcatttaacgaagccgtcaaagtgttaaccaatgtggccttggacaactttactttaccaggtacaccgggtttccctctgaataatgtttaccaagttccagtacaagatcataattcgatggacttgttgaagacttatattcagcaattccgtcaagaattggcaatgagactattagaaagagtctatagctctacagattctaaggaatatccctcaaagttttggttggcgttcacaagaagaagattcatgaacaaatctctataa

SEQ ID NO: 200 - DNA, 1701 bp, Saccharomyces cerevisiae
atggagtactggcattatgtggaaactacgtcatcgggccaacctcttcttcgagaaggtgaaaaagatatctttatcgatcagtccgtaggtctttaccatggcaaatctaagattctacagcgacagaggggcagaatctttttaacgtcacaaagaattatctacatcgacgatgcaaagcccactcagaactcgcttgggttggaattggacgatctagcgtatgtaaactattcatctggcttcttgaccagatcacctcgtctgatcctgtttttcaaggacccctccagtaaggatgaacttggtaaaagtgcggaaacagcaagtgccgacgttgtttcgacgtgggtctgccccatctgtatggtttcgaacgaaacgcagggggaattcactaaagacactctacctacacccatttgtatcaattgcggtgtgccagcggactatgagctcaccaagtcgtctataaattgttctaatgcaatcgacccgaacgccaacccacagaatcagttcggagtgaactctgagaacatttgccccgcctgtacttttgctaatcatcctcaaataggaaattgcgagatttgcggccacagattgcctaatgcatccaaagtacgatcaaagctgaacaggctaaactttcatgattctagggtccatattgagctagaaaaaaattcactggctagaaacaagagctcgcactcggcattatcgtcctcgtcctcgacaggatcatcgacagaattcgtgcaattgagtttccgcaaatctgatggcgtattgttttcacaagctacagagagggctttggagaatatactcactgaaaagaataagcatattttcaatcagaatgtggtttctgtaaatggtgtagacatgaggaaaggggctagttctcacgaatacaacaatgaggtgccatttattgagactaaattgagcagaattggcatatcaagcttggaaaaatctagagaaaaccagcttttgaacaatgacattctctttaacaatgcgttgaccgacctaaacaagctaatgtcgctggctaccagcatcgaaagactatacaaaaatagcaacataacgatgaaaaccaagacattgaacttacaggatgaatcaaccgtgaatgaaccgaaaacgagaagaccgctattgatacttgatagagagaagttcctaaataaagagctgttcttggatgagattgcgagagaaatttacgagttcacgttgtccgagtttaaagatttgaacagtgataccaactatatgatcataactctggtagatctatatgcaatgtacaacaaatctatgcgtataggtacaggcctgatatctcccatggaaatgagagaagcatgcgaaagattcgagcatttaggtctaaatgaattgaagttagtgaaagttaacaaaagaatattatgtgtaacgagcgaaaaattcgacgttgtaaaagaaaagctagtagatctgatcggtgataatcctggttctgatctcctaagattgacacagattttgagttccaataattcaaaatcaaactggacgctaggtatccttatggaggtattacaaaattgtgtcgatgaaggcgatttactgatagacaagcaactgagtgggatttactactataaaaactcttattggccctcgcatatataa

SEQ ID NO: 201 - DNA, 1152 bp, Saccharomyces cerevisiae
atgagtgaaacagaattgagaaaaagacaggcccaattcactagggagttacatggtgatgatattggtaaaaagacaggtttgagtgcattgatgtcgaagaacaactctgcccaaaaggaagccgttcagaagtacttgagaaattgggatggtagaaccgataaagatgccgaagaacgtcgtcttgaggattataatgaagccacacattcctactataacgtcgttacagatttctatgaatatggttggggttcctctttccatttcagcagattttataaaggtgagagtttcgctgcctcgatagcaagacatgaacattatttagcttacaaggctggtattcaaagaggcgatttagttctcgacgttggttgtggtgttgggggcccagcaagagagattgcaagatttaccggttgtaacgtcatcggtctaaacaataacgattaccaaattgccaaggcaaaatattacgctaaaaaatacaatttgagtgaccaaatggactttgtaaagggtgatttcatgaaaatggatttcgaagaaaacactttcgacaaagtttatgcaattgaggccacatgtcacgctccaaaattagaaggtgtatacagcgaaatctacaaggttttgaaaccgggtggtacctttgctgtttacgaatgggtaatgactgataaatatgacgaaaacaatcctgaacatagaaagatcgcttatgaaattgaactaggtgatggtatcccaaagatgttccatgtcgacgtggctaggaaagcattgaagaactgtggtttcgaagtcctcgttagcgaagacctggcggacaatgatgatgaaatcccttggtattacccattaactggtgagtggaagtacgttcaaaacttagctaatttggccacatttttcagaacttcttacttgggtagacaatttactacagcaatggttactgtaatggaaaaattaggtctagccccagaaggttccaaggaagttactgctgctctagaaaatgctgcggttggtttagttgccggtggtaagtccaagttattcactccaatgatgcttttcgtcgctaggaagccagaaaacgccgaaaccccctcccaaacttcccaagaagcaactcaataa

SEQ ID NO: 202 - DNA, 1179 bp, Saccharomyces cerevisiae
atgcctctgtcacaaaaacaaatagaccaagttagaaccaaagtgcactacagtgaagtagatactccattcaataaatatttagacatcttaggaaaagtaaccaagctgacaggaagcattataaatggcacattatccaatgatgatagcaagattgaaaaacttactgagcaaaatatctcccaactaaaagaaagtgctcatcttcgatttttggatctgcagtcatcaattgacacaaagaaagtagcagacgaaaattgggaaacatgccaacaggagacattggccaagctggaaaatctcaaagataaattgcctgatataaagagcatccatagcaagttgcttttacgtattggaaaactacaaggtctttacgattctgtccaagtaattaacagagaggtggaaggcttgtcagaaggccgtaccagccttgtggtgacacgtgcggaatgggagaaagaacttggcactgatctggtcaagtttttaattgaaaagaattatctcaaactagttgatccgggcctgaaaaaggatagttcagaggagagatatcgtatttatgacgatttctctaagggcccaaaagagttagaaagtatcaacgcctcaatgaaatcggatatagaaaacgtaaggcaggaggtatcgtcttacaaagagaagtggctaagagatgcagaaatatttggcaagatcacatcaatattcaaagaagaacttctgaagagagatggcctgctcaatgaggcagaaggagataacattgatgaagattatgaatcggatgaagatgaggaaagaaaagagaggtttaaaaggcagagatcaatggtggaagtgaatactatagaaaatgtggacgaaaaagaggaaagcgatcatgaatatgacgatcaggaggatgaagaaaatgaagaggaagatgatatggaagtagacgttgaggatataaaagaggataatgaagttgatggggaaagcagtcaacaagaagataatagtcgccagggtaataatgaggaaacagacaaagaaactggagtgatagaagagccagatgcggttaatgacgcagaggaggcagatagcgatcactcaagcaggaaacttggaggcactacaagcgattttagtgcgtcttcctctgttgaagaagtaaaatga

SEQ ID NO: 203 - DNA, 492 bp, Saccharomyces cerevisiae
atgtcctctcaaaaaatcgatttaactaaactaaatcccgagcagctgaatgctgtaaaacagcaattcgaccaagaattgcagcatttcacacagtccttgcaagcattaaccatggctaagggcaagttcacagaatgtattgatgatattaaaacagtctcccaagcaggaaatgaagggcaaaaactactggttccagcatctgcatcattatacatcccaggtaagattgtagacaataagaaattcatggtcgacattggtacaggatattacgttgaaaagagcgctgaagcagcaatcgcattttaccaaaagaaagtagacaagcttaacaaagaatctgtacagatccaagatattattaaggaaaagacccagtattccttgtccatcgaggcccaaataagacaagccgctattagacagcatgaagcgatgagtaaacaacaacagcagcaacaaaaaaaggagtcgtctacagcctag

SEQ ID NO: 204 - DNA, 891 bp, Saccharomyces cerevisiae
atggactctcttgaagctagattacaattcattcaggtcctgaagaacctgcaaaagacgctgcacaagaccagagactctatcacatcatcgtcgaccaccacaccaccgtcatcgcaacaaaagctgaacaatgaccctatacagttctacttgagaaactacagacatcactacgaggacttccaccaatgtttgttcgatacaaccatgaagatggacccactagatagactggacgtagtgatatactatgttagaataataagaaacttatatccgcatagccattccaataccaatgttacaaaagtgttaaacgaagtgctactcatggacatagacttggtttttgagctttgtctgccctgccaggactggaaatccctcacgaatcaagccacctgtaaagagctattccttgacttatccaaactaatccattacgacgccaccagcgtcacgcacacaccatcagacaccacactcatagacgctactacctggtacagtgtcaagacagagaggactacaaaggactataaagaatcattgcaacgaacggagtctctgcttaaggacagagacttgaagaaattggctttctttcagcagttcaattccgatacaactgcgatcaacccggacttacagacgcagccaaccaatgcaaacattttgttgcacaggatggaagcagacagggaactgcacaagaggtcgaaagagacaagttggtacatcgaaaggccctccaacgatatactggatgaatccgaatttaaaagcttgtggacgcattttgaaactactgattccggatttgataaggacgattacaaaaatatcaaggctttaaatgacattgcgaaggcatcttacatatattag

SEQ ID NO: 205 - DNA, 2010 bp, Saccharomyces cerevisiae
atgagctacagttatgaagcttgtttttgggacccaaacgacaatggtgtgaacatccttctgggtcacatttctcagggaataagatcttgcgactcaatgatacttttctttaagcagcgtagtgagcttgagaaggactatgccaggcggcttggagccatcacgggaaaactagacaaagacattggaacaaacatggattatggaaagttgaatgaaacatttaatgtggtgctcagtgttgagaaagctcgagcacaatcgcattccaagcaaagtgagattcttttcagacagatttacacggatactaaggcatttgccgctaacttgcaagcaagatatactacattgagtggaaagattgaaaggttgcggatggacaagttcaacaagaaaaaggggtgtgaggtgttgcaaaagaaattacaggatgcccagattagattcagagacttgcaattgaacgagaataatatgattggggccaagagagtagaacacaacaagagagaactattgaaatgggaatcgaactctcaggaatataaggttcaactagacgttcttaagcaagaatataaggcatcgcagaaattctggatacacgagtgggcgcaattatcctgcgaactgcaagaaatggagaacgctagaatatcctttttacaatcgaaattacaacagttcgcaacctcatcgatggaaacatatattttagagcaaacaaaaatggacatgttaacaaatcatctgaactcattcacggcagcggatgaaatatcgacgttttcaaaagaaaatggaactggtagactgaaacataagacttccaaaggtgacatgaattctagcgccaactgggctcaaatgagcagtatctccacgacaagtaaaaaaaccgaatcgtatatggacaatataaggaaactatcttctcagctgaaggaaaccgaaaataaaaggaaactagcttcaatagacaaatatgagaaaccattgccatctccagaagtcaccatggctacacaatttagaaattccacacccgtcatacgtaatgagacaaaagtggttgctaacccaacattatctttgaggtcttcacccgtccagttacagagcaatgtggatgactccgtgttaaggcagaaacctgataaaccaaggccgattgttggggaagaacaacttaaacctgatgaagattcgaaaaatcctgatgaaaaaggtctaatggtgcataaaagaaatcaatctctcagctcaccatcagaatcaagttcttctaatccaacggattttagccacatcaaaaagagacaaagtatggaatctatgactacatccgtaagttcaatggccaatagtatagacgactcacagagatttgccaaatcatggaactcgtcaaataggaaaagaaaatctatgagccatttgcaagtaccctcatccgcttcttcgaggtcggatgatgggggaagaacgcctaactctgcgcacaacctcaatgaagatgattataacacgaggagagataccagtacaagcacaattttgttcaaacctcctgtggcagtaagaggaacgtctagggggcacacgcatagacaatccatgataatgcaagattcaagtaatccaattgaagatgccttgtacgaaatggaaagaatccaaagtagttctaaaccaggtacaaaaacaggaaacatcatggatgagagaggtgtggtcagggatagaggtattactgttaccttgcctattgtcaccagtgaaggttttccagtcattgaatatgccaaggccatgtatccattgattggaaatgaggcacctggattagcgaatttccataagggcgactatctgctaattactgagattgttaataaagattggtataaaggtgaagtttatgataatgaccgtatagatagaaatcataggataggattaattccctataatttcattcagctactgcatcaaggtctttga

SEQ ID NO: 206 - DNA, 666 bp, Saccharomyces cerevisiae
atgggccaaaaaagtagtaaagtgcatataaccaaaacagatagggcgatcctagaagtaaaacgatctaaagatgaaattcataagtttacgaggaggacagataatttgattttggtagagaagagtcaactaaaagacctaatacgaaaaaatcctgaaaattataaatctaacatgaaggtacgatttctactaaaaagaattcattatcaggagcatttgttgcagcaggcttcggatcagcttattaatttggaaaatatggtgtcaacacttgagttcaaaatggtagaaaagcaatttattaatggactgaaaaatggaaatgaaatcttgaaaaaactaaacaaggaatttagcaatgttgatgaactgatggacgatgtacaagatcaaattgcgtatcaaaatgaaattaacgagacattatctagaagcttagtcggaacaagcaattatgaagacgatctagataaggaattggatgctctagaaagtgaactcaatccagagaagatgaataacgcaaaagtagctaacatgccatcaacagaaggtttgccatcattgcctcaaggagaacaaaccgagcaaaaagaaagagaggaattcgcgacggaggagagatcagacactaaggaacctttagcattactatcctga

SEQ ID NO: 207 - DNA, 669 bp, Saccharomyces cerevisiae
atgaagtttttcccactccttttgttgattggtgttgtaggctacattatgaacgtattgttcactacctggttgccaaccaattacatgttcgatccaaaaactttgaacgaaatatgtaactcggtgattagcaaacacaacgcagcagaaggtttatccactgaagacctgttacaggatgtcagagacgcacttgcctctcattacggggacgaatacatcaacaggtacgtcaaagaagaatgggtcttcaacaatgctggtggtgcgatgggccaaatgatcatcctacacgcttccgtatccgagtacttaattctattcggaaccgctgttggtactgaagggcacacaggtgttcactttgctgacgactattttaccatcttacatggtacgcaaatcgcagcattgccatatgccactgaagccgaagtttacactcctggtatgactcatcacttgaagaagggatacgccaagcaatacagcatgccaggtggttcctttgcccttgaattggctcaaggctggattccatgtatgttgccattcgggtttttggacactttctccagtactcttgatttatacactctatatagaactgtctacctgactgccagggacatgggtaagaacttgttgcaaaacaaaaagttctaa

SEQ ID NO: 208 - DNA, 606 bp, Saccharomyces cerevisiae
atggctaggccagttaatacaaacgctgaaacggaatcgagaggcagacccactcagggtggtggttacgcaagtaacaacaatggcagctgtaacaacaacaatggcagcaataataacaataataacaataataataataataataacagcaataacagcaataacaataacgggcctacctccagcgggagaaccaatgggaagcaaagactcacagctgcacaacagcagtacattaagaatctcatagagacacatatcaccgacaaccaccctgatctgcgccccaagagtcacccaatggatttcgaagagtatacggatgcgttcctgagaaggtataaagaccactttcaattggacgtaccggacaacctgacgctgcagggatacttattgggttccaaattgggcgcgaagacttattcatacaagaggaacacacaggggcagcacgataaaaggatacataagagagacctggccaacgtggtgagaaggcatttcgatgagcattccattaaagagacagattgcataccgcaatttatatacaaggtaaaaaaccagaagaagaaattcaagatggaatttcggggttga

SEQ ID NO: 209 - DNA, 3498 bp, Saccharomyces cerevisiae
atgacagaagaagatagaaagctcactgtagagacagaaacagttgaggcacccgtggcaaataatcttttattgtcgaataacagtaatgtagtagcacctaatccttctattccctctgcctccacatctacctctccgctacacagggaaatagttgatgattctgtcgctactgctaacaccaccagcaacgttgtacagcataatttgcccaccatagataacaatttaatggattccgatgccacgtcacataatcaagatcattggcattcagacataaacagggcaggaacatcaatgtcaacgagtgatatcccaacagatttacatttagaacatatcggctctgtttcatcaactaataataatagtaacaatgccctaatcaaccacaaccctctgtcatctcatctttccaatccgtcatcttcattacgaaacaagaagagctctttgttggtagcttctaaccctgcgtttgcttcagatgttgaactctcgaagaagaaacctgccgtcatctccaataatatgcctacaagtaacattgccctttatcaaacagcgagatcggcgaatattcatggtccatcatcaacttccgcatctaaagcgttcagaaaggcttcggccttctccaataacacggcacccagcactagtaataacatcggctcgaatacacctccagctcctcttttacctctaccttcactatcacaacaaaataagccaaaaataatagagaggcccacaatgcacgtcactaattcaagagaaatacttttaggtgaaaacctgttagatgatacaaaggcgaagaatgctcccgcgaattcaaccacacacgataatggtccagtagcaaatgatgggctgcgtataccgaatcactcgaacgcagatgataatgaaaataacaacaaaatgaagaagaataaaaacattaatagtgggaaaaatgaacgtaatgatgacaccagcaaaatatgcactacatctactaaaacagcgccttcaaccgcacctttgggcagtacagacaatactcaggctctcactgctagtgtctccagcagtaatgctgacaatcacaataataacaagaagaaaaccagcagcaacaacaacggcaataacagtaatagtgcatccaataaaaccaatgccgatatcaagaattctaacgccgacttgagcgcttctacctctaacaataatgcaataaatgacgactcacatgagagtaattcagaaaaaccaacaaaggcggattttttcgctgcaaggctggctacagctgtaggtgaaaatgaaattagtgattctgaggaaacatttgtttatgaatcggcagctaattcgactaaaaacctaatatttcctgactcctccagccagcagcagcagcagcaacagcaacctccaaaacaacagcaacagcaacaaaatcatggaataacctcaaagataagcgccccattgctaaacaataacaaaaaattattaagccgactgaaaaattcaagacatattagcactggtgccatattgaataacacaatcgcgactataagcacaaatccgaacttgaattctaatgtgatgcagaacaataacaatctgatgtcgggacacaatcacctggacgagttgagcagtataaaacaggagccaccgcatcaattgcagcagcagcaaccaccaatggatgtacaatcggtagattcgtatacctctgacaacccagacagcaatgttattgccaagtcgcctgataagaggtcaagcttagtatccctatctaaagtttctccacatttactttcatccacatcaagcaacggtaacacaatatcgtgtcccaatgttgccacaaattcgcaggaattggaaccaaacaatgatatttcaacgaagaaatctctttccaattctactttgagacattcctctgctaatagaaattctaattatggtgacaacaaaaggcctcttagaacaacagtgtcaaagatatttgattcaaaccctaatggagctcctttacggagatactctggggtaccggatcacgttaatctagaagattacatcgaacagccgcataattatccaacaatgcaaaatagtgtgaaaaaggatgaattttataacagcaggaacaataagtttccccatggtttaaacttttatggtgataacaatgttattgaagaggaaaataatggtgactcgtctaatgtaaatcgaccgcaacacactaaccttcagcatgagtttattccagaagataacgaaagtgatgaaaacgatattcactccatgttttattataatcataagaacgatttagaaacaaaaccgctaatatccgattatggtgaagatgaagacgtagatgattatgatcgcccaaatgctactttcaacagttactatggctcagcatccaacacgcacgaacttccattacatggaaggatgccttcaagatcaaataatgattactacgattttatggttggcaacaatactggcaataacaaccaattgaatgaatatacccccttaagaatgaaacgtggtcaaagacacctatcaagaacaaacaatagcataatgaatggtagcatccatatgaatggtaacgatgacgttacccattccaatatcaataataacgatattgttggttactcaccgcacaacttttactcaaggaagtccccatttgtgaaagtaaagaattttctttatcttgcatttgttatatcatcactattgatgacaggattcattctgggatttttattggccactaataaagaactacaagatgtagacgtggtagtgatggataatgtgatttcaagttcggacgagttgatcttcgacatcacagtaagtgcttttaatccaggattcttcagtataagcgtttcccaagtcgatttggacatttttgcaaaaagttcttacctgaagtgcgattctaatggtgactgtacagtaatggaacaggaacggaaaattttacaaataacgacaaatctttcgttagttgaagagagtgctaataatgatattagtggtgggaacatagagacggtattactaggaaccgctaaaaaactagagacaccattaaagttccagggcggcgcatttaataggaactacgatgtgtcagtctcgagtgtcaagcttttaagtcctgggtctcgtgaagccaagcacgaaaacgacgatgatgacgatgatgatggcgacgatggtgacgatgaaaacaatactaatgaaagacaatacaaaagcaaaccaaatgctagagatgacaaagaagatgatactaaaaaatggaagctactaatcaagcatgattacgaattgatagtccgtggaagcatgaagtatgaggtgccctttttcaatacgcaaaaatctacggctattcaaaaggattccatggtccatcctggtaagaagtga

SEQ ID NO: 210 - DNA, 1230 bp, Saccharomyces cerevisiae
atggttaaagaaactaagttttacgatattctaggtgttccagtaactgccactgatgtcgaaattaagaaagcttatagaaaatgcgccttaaaataccatccagataagaatccaagtgaggaagctgcagaaaagttcaaagaagcttcagcagcctatgaaattttatcagatcctgaaaagagagatatatatgaccaatttggtgaagatggtctaagtggtgctggtggcgctggcggattcccaggtggtggattcggttttggtgacgatatcttttcccaattctttggtgctggtggcgcacaaagaccaagaggtccccaaagaggtaaagatatcaagcatgaaatttctgcctcacttgaagaattatataagggtaggacagctaagttagcccttaacaaacagatcctatgtaaagaatgtgaaggtcgtggtggtaagaaaggcgccgtcaagaagtgtaccagctgtaatggtcaaggtattaaatttgtaacaagacaaatgggtccaatgatccaaagattccaaacagagtgtgatgtctgtcacggtactggtgatatcattgatcctaaggatcgttgtaaatcttgtaacggtaagaaagttgaaaacgaaaggaagatcctagaagtccatgtcgaaccaggtatgaaagatggtcaaagaatcgttttcaaaggtgaagctgaccaagccccagatgtcattccaggtgatgttgtcttcatagtttctgagagaccacacaagagcttcaagagagatggtgatgatttagtatatgaggctgaaattgatctattgactgctatcgctggtggtgaatttgcattggaacatgtttctggtgattggttaaaggtcggtattgttccaggtgaagttattgccccaggtatgcgtaaggtcatcgaaggtaaaggtatgccaattccaaaatacggtggctatggtaatttaatcatcaaatttactatcaagttcccagaaaaccatttcacatcagaagaaaacttgaagaagttagaagaaattttgcctccaagaattgtcccagccattccaaagaaagctactgtggacgaatgtgtactcgcagactttgacccagccaaatacaacagaacacgggcctccaggggtggtgcaaactatgattccgatgaagaagaacaaggtggcgaaggtgttcaatgtgcatctcaatga

SEQ ID NO: 211 - DNA, 1050 bp, Saccharomyces cerevisiae
atgcccaagttggaacaatttgaaataaaaaaatactggcaaatcttctcgggtttgaaaccaatagagaataaggtaaaccatgaccaggttttaccaattctttataattccaaattagattcatcggttctaaacaagatttggtttttagctgatattgatgacgacgacaatttagactttgaggaatttgtaatttgcatgagattaatatttgatatggttaataaaaacattagctctgttccagatgaattgcctgattggttgattccagggagtaaggtgaatctaatcaaagaaagaaagaagcgcaagcaaatagaaaacgcagacttgcctccaaagaaggaaatcaaagtagattggtacatgtctccagatgatttgaatcaatatgaaaaaatttataatagctgtgcaaaactaaccgatggtactattacattcaacgaactgtcaacaaaactttccacaaaattttttaacatcagtaagacagatttaaataaggtttggagtttaattaacccacaaaatttgccatcaattgatagagatcctactttttattttattcactgtttaagacaaagaaatgatttgggtgctgaaattccagcaagtttaccaaattctctggcggaggtgtgcaataaaaaacaactgagctatgatttacggtcctctcaacctcctacaaagagaaaagaagaagctaacgaagtggacaatctgcgtgacaatgggcagaactcgagttccgatagcagtggcagtaacgtgctttcaaatgaagacagtataaagcaaaaatatgcttctctgacagatgatcaagttgcgaacatgagagaacaattagaaggccttttgaactacaaaaagagtgaaaaaactcaaggaggctcaaaactttctaagcggattaacatcaggtcgataactgatgacttggataacattgaacagcaggttgaagtattggagaattacttgaacaataagagacacgaattgcaagcattacaagcagaaatcaattga

SEQ ID NO: 212 - DNA, 993 bp, Saccharomyces cerevisiae
atgagttcaccagcgaacctattccctggattaaacgacataactgacgtgctggaggaattcccactggccacctccagatatttaacattactacacgaaatagatgcaaaatgtgtgcattctatgccgaatttgaacgagaggatagataagttcttgaagaaagacttcaataaagatcaccaaacacaagtaagactgctcaataatatcaacaagatttatgaagaactgatgccatcgctggaggagaaaatgcatgtctcatccattatgctggataatctagacagattgacgtcccggttagaattggcgtatgaagtcgcaatcaagaacacagaaattcctagaggtttaagactgggtgtggacaaccatccagcaatgcacctccatcatgaactaatggaaaaaatagagagcaaatcaaacagcaaatcgtcgcaggcactgaagagcgaatcaagaagagaagccatggctgccaacaggaggcagggcgaacattactccgccagcacacaccaacaagacgactcaaagaacgacgcaaactacggaggcagcaggcacgagagccaagaccacactggtaacaacacaaactcaagaaaaagagccaacgctgccaataccaacaacgccgatccagagaccaaaaaacgcaagaggagagttgccaccacagccgtttcaccaagcactatcagcacggcaactgccgtcaataatggcaggataggtacatctacagcgtccaggggagttagcagcgtcggaaacagcaacaacagcaggatatcaagaccaaaaaccaacgactacggcgaaccgctctactgctactgtaaccaagtggcatacggggaaatggtggggtgtgatggcgcagactgtgagctagaatggttccatttgccatgtattggactcgaaactctacctaagggcaagtggtattgcgacgactgcaaaaaaaaactgtga

SEQ ID NO: 213 - DNA, 522 bp, Saccharomyces cerevisiae
atgtcatctaccagtgataataatgctaatagtgcaagagagaaaaaaccactgaaattcgtgtcagaaggagttggtaatgtagaggcccagaggatacgagagcaagtggagcaaaaaaaatatgaagcggaatataaaaggaaaaccaggaagtccctgcgagatcagcttaggtctaatgctattagtaaacaaaaacaatataacggtttggtgagagaccgtgaaagttttactaggttgagtaaagaggacctggaattttaccagaagagcaagaatgagttgctaaaaaaggaaaaagaactgaataactacttagacgtcaaggcaatcaattttgaaaagaaaaaaaaagcattactaatggagaacgattctactactaatacagaaaaatatttagagaccggcactagtttgggcagtaaaacacaaataaagggcgtaaagacatccagccctaaaccaaaaatcaaggtatccataaaaaaattgggtagaaaactggagaactga

SEQ ID NO: 214 - DNA, 570 bp, Saccharomyces cerevisiae
atgctggaggcaaagatggttatggtagttccaactatagaggaccaagcaatgatcgttctgcacccaaaaatattaagccaatcagtagttatgcacataaaaggtcagaactaccaactagacctagtaaaagtaaaacatataatgatagaagtagggcacttagacccacaggtccggacaggggcgatggttttgatcaaagggataaccgcttacgtgaagagtataagaaaaacagttcacaacgttcacaattacggtttcctgaaaagccatttcaagagggcaaagacagcagcaaagccaacccttaccaagcttcatcttacaagcgcgactcgccttcagaaaatgaagaaaagccgaataaaagattcaagaaggatgaaacaattagaaacaaatttcaaacgcaagattatagaaatacacgagatagcggtgctgcccatagagccaatgaaaatcaaaggtacaacggaaatagaaagagcaatactcaggcgcttccgcaaggtcccaagggtgggaattacgtcagtaggtaccagaggtaattactag

SEQ ID NO: 215 - DNA, 348 bp, Saccharomyces cerevisiae
atgcatcagcaacactccaaatcagagaacaaaccacaacagcaaaggaaaaaattcgaaggccctaaaagagaagctattctggatttagcgaagtataaagattctaaaattcgcgtcaaattaatgggtggtaaattagttataggtgtcctaaaaggctatgatcaactgatgaacttggtacttgatgatacagtagaatatatgtctaatcctgatgatgaaaacaacactgaactgatttctaaaaacgcaagaaagctaggtttgaccgtcataagaggtactattttggtctctttaagttccgccgaaggttctgatgtactatatatgcaaaaatag

SEQ ID NO: 216 - DNA, 1065 bp, Saccharomyces cerevisiae
atgatgaataacaacggcaaccaagtgtcgaatctctccaatgcgctccgtcaagtaaacataggaaacaggaacagtaatacaaccaccgatcaaagtaatataaattttgaattttcaacaggtgtaaataataataataataacaatagcagtagtaataacaataatgttcaaaacaataacagcggccgcaatggtagccaaaataatgataacgagaataatatcaagaataccttagaacaacatcgacaacaacaacaggcattttcggatatgagtcacgtggagtattccagaattacaaaattttttcaagaacaaccactggagggatatacccttttctctcacaggtctgcgcctaatggattcaaagttgctatagtactaagtgaacttggatttcattataacacaatcttcctagatttcaatcttggcgaacatagggcccccgaatttgtgtctgtgaaccctaatgcaagagttccagctttaatcgatcatggtatggacaacttgtctatttgggaatcaggggcgattttattacatttggtaaataaatattacaaagagactggtaatccattactctggtccgatgatttagctgaccaatcacaaatcaacgcatggttgttcttccaaacgtcagggcatgcgccaatgattggacaagctttacatttcagatacttccattcacaaaagatagcaagtgctgtagaaagatatacggatgaggttagaagagtttacggtgtagtggagatggccttggctgaacgtagagaagcgctggtgatggaattagacacggaaaatgcggctgcatactcagctggtacaacaccaatgtcacaaagtcgtttctttgattatcccgtatggcttgtaggagataaattaactatagcagatttggcctttgtcccatggaataatgtcgtggatagaattggcattaatatcaaaattgaatttccagaagtttacaaatggacgaagcatatgatgagaagacccgcggtcatcaaggcattgcgtggtgaatga

SEQ ID NO: 217 - DNA, 2925 bp, Saccharomyces cerevisiae
atgatgcttggagagcatttaatgagctggtctaagactggtattattgcatacagtgactcacagtcctcaaatgccaatatttgcctcacatttttagaatccataaatggaataaactggcgatttcatacgccgcaaaaatatgtattacacccgcaattgcacgaagttcaataccaagaaagtagtagtactctaagcactcactcgacaacaacttctgtcaatggatcaactacagccggggttggaagcactccaaacttcggaggcaacagcaacaagtctcctccacagttcttttataatatatcttccattcattggaacaactggtttagtttaccaggtgacatgttggctgtatgtgacgaattaggtaacatgaccatgttaattactggacaacgtcctgatcgggccactacatacgaaaagttaacgatggtatttcaagataatgtttacaaaatttataaccacgttatgcctttaaagcctgtagacaagctaaagcccatgaacattgaaaggaaacaaacaaggaaagaatacaatacatccatcttggaatttcgctggctcacctcttcaaaatccgtaatcgtttctcaattctgcgcatttgatagtagttctaatacatatagaagcagagcacagcaagtgcctccatatggtgtgtaccatccaccctttataaaatatgcgtgcttggcaataaggaaaaacggtcaaatagatttttggtaccaattttcaaactcaaaggatcataaaaaaataacgttacagctgctggacacttcaaatcaaagattcaaggatcttcaatggcttgaatttgctagaattacacctatgaatgatgatcagtgtatgttgataaccacatattcaaaattatctaaaaatatctctttttacaaactacacgttaattggaaccttaatgcgaccaaaccaaacgtcctaaatgacccatcgttgaaaatacaatttatattaagcactactttagatccaacggatgatgaagggcatgtgttgaagttagagaacctgcatgttgtgtctaaatcatcaattgagaaagatccctccccagaaattttagttttgtataatgtttgcgatacatcaaaatcattggtgaaaagatatagactagctccaacacaactttcagctgagtatttggtgatattgaaaccagatttaaatatcgatcgaaataattctacgaatcaaatattccaatcgcgccgttacaaccttcgtcgccacagtgatatcgttctcgacaaaaaagtcaccttgataacttctgagatgttcgatgcatttgtttcattttactttgaagatggtaccatcgaatcctacaatcaaaatgattggaagttggagactgaacgacttataagtcagagccaattaggaaaattcaaaaatattattgcatccccactaagcgcaggatttaattatgggaagctccctttacctccatcggttgagtggatgaaggtttcaccatctatgtgtggggttatcgttaaacagtataataaaaaatggccacagttttatgctgctgttcaaaaaaactacgcagatcctgagaaggattctataaatgctacagctttggcatttggatacgtgaaaagtttacataagcaaatatctgccgaagacttgaccatcgctgcgaagacgcacatactcagaatttcattcttggatagaaaaagggccaaagaatttattacgactttgttgaaaagcctatactcgtttttcaacatttctcctgatgcgcctaaagagattatggataaaataataacaagtaggccacttcaaaaaattatgctattgcagctggaacttggcagttgtttttcgcaagaaaatattgaagaaatggcgagagttatactctacttgaaaaatgttttgtttgcgttcaatggtgtggcgagaaattttcattttgcgattgaacaaataagtaataattctaaccaacaacaaaacccgaagttgttccagactatattttcaaaacaggatctcattcattcattgattcctgttgctaaatggtttgtcaaatttatcacttatttgacccaagaaattttaattttaataaatgatcccacaaacaaggaatacacactagttcacggaatatttggagccaaaatgtccaggactttgatattgtctattcttaacgaaatcaagaaagtcacccaaatagtggcgaaatttccagaaacatcatacccaattttaaacgagtcatcaacttttttgaaactagtcttatctgaatcaccagtggatttcgaaaaatttgaaacgtttttagttgatgtaaacaacaaatttatcgcgctatgcgaacaacagccatcacaggaacgtgagttttcgttgttggtgaaagctgaaatccccccagaatatgccaaagtgggagatttcctcctgcagtatgctaataatgcagtgatttcccatgcgaatgcggctgcagtatattttgcagatacaagtggcttgaaaatatctaattcagagtttttcaatcccgaaatatttcatttattgcaaccattagaagagggattaattattgatacagataaactacccataaaaaatagaacctccaaatcttttagcaaattactgtacgacgatgtaacctgtgacaaactgagtgtatcagaaatatccgatgggaaacttaagagatgcagtaggtgcggttccgtcacaagagcaggaaatataatatcgagtgataagacaattgtacccactagcatccaaaccaaaagatggccaaccatgtatacaagactgtgcatatgttcaggtatgctttttgagatggacggctga

SEQ ID NO: 218 - DNA, 1548 bp, Saccharomyces cerevisiae
atgggtgttaccgttcaagacatatgttttgcctttttgcaaaactactacgaaagaatgaggaccgacccttccaaattggcatatttttatgcaagtacagccgagttgacgcatactaattatcagtccaagtcaacaaacgaaaaagatgacgttcttccaacggtgaaagttactggacgtgaaaacattaataagtttttttctagaaatgatgccaaagttaggagcttgaaactaaaattagatactatagatttccagtacactgggcacttgcacaaaagcattctgatcatggccacgggtgaaatgttttggacaggaactccggtctataagttttgtcaaacatttatcctgctaccatccagcaatgggtctacgtttgacattaccaatgatataatcagatttatttccaattcattcaagccttatgtcttaacagatgcttctttgtcacaatctaacgaggagaactctgtcagtgcagttgaagaagataaaataaggcatgagtccggagttgaaaaagaaaaagaaaaagaaaaatcaccagaaatatcgaaaccaaaggccaaaaaggaaacggtcaaagacacaactgcgccaactgaatcgtcaactcaggaaaaaccaattgtcgatcatagtcaacctcgggctatacccgtcactaaagagtccaagattcacaccgaaacggtaccatctagtactaagggcaaccacaaacaagatgaggtctctacagaagagttaggaaacgttactaaactaaatgaaaaatctcacaaagccgaaaagaaagctgcaccaatcaagacaaaggaaggctctgttgaggccataaatgctgttaataactcttctttgcccaacggtaaagaagttagtgatgaaaaaccggtacctgggggcgtaaaggaagctgaaactgaaattaaaccaatagagccacaggtttccgacgccaaagaatccggcaataacgcttcaacaccttcatcttctccagagcccgttgcgaatccaccaaagatgacatgggcctcaaagttgatgaatgaaaactccgacagaattagcaaaaataataccacggttgaatacatcagacctgaaacactacccaagaaaccgactgaaagaaaatttgaaatggggaatcggagggacaacgctagtgctaacagcaaaaataagaaaaaaccagtattcagcaccgttaacaaggacggcttttatccaatctacattagaggtacaaatggcttaagagaagaaaaattgagaagtgccttagaaaaagaattcggtaaagttatgaggataacagctgcagacaattttgccgtcgtcgactttgaaactcaaaagagccaaatcgatgctttagagaagaagaaaaagtcaattgatggtattgaagtatgcttagaaagaaaaacagtaaagaagcccacgagcaataatcctcctggcatatttactaatggaacacgttctcatagaaagcaacccctaaaaagaaaggactag

SEQ ID NO: 219 - DNA, 1302 bp, Saccharomyces cerevisiae
atgcaatctatgaatgtacaaccgagggtattagctgtaggaggtgagcagtttttctctcagaggcaggcttctgaacaacaccagcagcaaaacatgggacctcaggtgtattcgccaaaggttaatagagcaagaatgtttcctcaagggatgcctgtaaatacaattaatggctctgttaatcaggagatgaacaatgcctatcttttgaagcagaagaacgagccactgttaactcagcaacaacagcaacagcaacaacaacagcagccatttaatataggaacgcctgtttcggtggctagtttgccgcccggactgaacgttctgcagcaacaacaacagcagcaacaacaacagcagcaacagcagcagggagtgggactcaatcgtcccttggcttcccagctacccaagcatttaaccaaccaaagtatgccaccgatctttttaccacccccaaattatcttttcgttcgcgatgtctggaagtccaacctttacagtgaattcgccgttataagacaactggtctcgcagtataatcatgtgtcaatcagtactgagtttgtggggactttggctaggccgatcggcactttcaggtcgaaggtcgattaccactatcagacaatgagggccaacgtagacttcttaaacccaatacaacttggtctttccttgagtgacgccaacggcaataagcctgacaacggtccctcaacgtggcagttcaattttgaatttgacccaaagaaggaaatcatgtccacagaatcgttagaattactcagaaaatcaggtatcaacttcgaaaaacacgaaaatttaggcatcgatgtatttgaattttcgcagcttctaatggactctggtttaatgatggatgattctgttacttggatcacataccatgcggcatatgatctaggtttcctgatcaacattctaatgaatgactccatgcccaacaacaaggaggatttcgagtggtgggtccatcaatacatgcccaatttctacgacttaaacctggtgtataaaataatccaggagttcaagaaccctcaattgcaacaatcttcacaacagcaacagcagcagcaatattcactaaccacacttgccgatgagttagggctaccaagattctctatatttactactacaggcggccagagtctattgatgcttctatccttctgccaattgagcaagctgtctatgcataagttcccaaacggtacggatttcgccaagtaccaaggtgtcatatacggtattgatggggaccaatga

SEQ ID NO: 220 - DNA, 4611 bp, Saccharomyces cerevisiae
atgtcacaggtttggcataattcgaattcgcaatcaaacgatgtggctacttcaaatgacgctacgggttccaacgaaagaaacgaaaaagaaccgtccctccagggaaataagcccggttttgttcaacagcagcaacggattactttaccctcgctatctgccttgagtactaaggaggaagatagaagagattccaatggccaacaggctctaacttctcatgctgctcacatattaggttatcctcccccacattcaaatgctatgccctcaattgcaactgattcagcattgaaacagccccacgagtatcaccctcgccctaaatcttcgtcctcttctccctctataaacgcttcgcttatgaatgctggtccagctcccctccccacagtgggagccgccagtttttctttgtcgagatttgacaatccattaccgataaaagctcctgttcatacagaggaaccaaaaagttataatggtcttcaggaagaagaaaaggcgacgcaacggcctcaagattgcaaggaagttcccgctggtgtgcagcctgctgatgcccctgaccctagcagcaaccatgcagatgctaacgatgacaataataacaacgaaaattctcacgatgaagatgctgactacagacctctaaacgtgaaggatgccctatcttacctcgaacaggtcaaatttcaatttagttcgcgtccggatatctataatttatttttagatattatgaaggactttaaatctcaggcaatagacacaccgggcgttattgaaagagtatccactttgttcagaggttatccaattttgattcaagggttcaatacttttctaccccaaggctatagaatcgaatgctcctctaatccggacgaccccattagagttactacaccaatgggtactacgacagtaaacaataacatcagtccatctggtagaggtacaacggatgcacaggaacttggttcttttccagaaagcgatggaaatggtgttcaacagccctccaatgtgccaatggtgccttcgagtgtgtatcaatcggaacaaaaccaagaccaacaacaatctttgcctcttttagctacttcttctggtttaccttcaattcaacaacctgaaatgcctgcacatcgccaaatcccacaaagtcaatctttagtgcctcaagaagatgctaagaaaaacgttgatgtcgaatttagtcaagccataagctacgttaataaaattaaaactagatttgccgaccaacctgatatttacaagcattttctggaaatactacaaacttatcagcgagagcaaaagccaataaacgaagtctacgcacaagtgacgcatcttttccaaaatgctcctgatttactagaagatttcaagaaattcttgccggactcttcagcttctgccaatcagcaggtgcaacatgctcagcaacatgctcaacaacaacatgaggcccaaatgcatgcacaggcacaagctcaggctcaggcacaggcacaggtggaacaacagaagcagcaacagcaattcttgtatccagcttcgggctattacggccatccttctaaccgaggtattccacagcaaaacttgcctcctataggaagcttctcgcctccaacaaacggttcgaccgtacatgaagcctatcaggatcaacagcacatgcaaccaccccacttcatgcccttaccatcgatcgttcaacacgggccaaatatggtacatcaagggatagcgaatgaaaatccacctctatcagatctaagaacgtctctcactgaacagtacgctccttctagtatacaacatcagcagcagcacccgcaaagtattagtcctatagcaaatacgcagtatggtgatatccctgttagaccggaaattgatttagatcctagcattgtgcctgtggtccctgaacccactgagcccatcgaaaacaatatatcgcttaatgaggaagtcactttcttcgaaaaggccaagaggtatatcggcaataaacatttatacactgagtttttgaaaattttaaatttgtactctcaagatatacttgatcttgacgatttagtggaaaaggtagatttctacttgggttccaataaagaactatttacgtggttcaaaaactttgttggctaccaagaaaaaaccaaatgtatcgagaatattgttcatgaaaaacatagattggatttagatttatgtgaggcatttggcccaagttacaagaggctaccaaaaagtgacactttcatgccatgctcaggtagggatgatatgtgttgggaagtcttgaacgatgaatgggttggacatcctgtatgggcttccgaagattcgggatttattgctcatcgtaaaaaccagtatgaggaaacactattcaagatcgaagaggaaagacatgagtatgatttttacattgaatcaaatttaagaactattcaatgtttggaaacaattgtaaataagatcgagaacatgactgagaatgaaaaagccaattttaaactgcctccaggtcttggccatacttcaatgactatttataaaaaagtgataaggaaagtttatgataaggaaagggggttcgagattattgatgctttgcatgagcaccctgcagtgacagccccagtcgttctgaaaaggttaaagcaaaaagacgaagaatggagaagagctcaacgtgaatggaataaagtttggagggagttagaacagaaggtttttttcaagtcattagatcatttaggcttaacatttaaacaggctgacaagaaattattaactacaaagcagttgatatcagagattagcagcatcaaagttgatcaaacaaacaaaaaaattcactggttaactcctaaaccaaagagccagttagattttgatttccctgataaaaacattttctatgatatcttgtgtttggctgacacttttataacccataccacagcctattctaatcccgataaagaaagattgaaagatttactaaaatacttcatctctttgtttttttctatttctttcgaaaaaatcgaagaatcgttgtactcccataagcaaaacgtgtcagaatctagcggatctgacgatggcagttctattgcatcaaggaagaggccctatcaacaagaaatgagtttacttgatattttacacaggagcagatatcaaaagctaaagcgttctaatgatgaagatggcaaagttccccagctctctgaaccacccgaagaagaacctaataccattgaggaggaagagctcatcgatgaagaagctaaaaatccgtggctaactgggaatttagtggaggaagcaaactctcagggtattatccaaaatcgcagtatttttaatctattcgctaatacgaatatttacatatttttccgtcattggacaacgatttatgagcggcttttggaaattaagcaaatgaatgaaagggtcacaaaggaaatcaacacaaggtcgacagttacttttgccaaagatctagatttattatcgagtcaactttccgaaatgggactagattttgttggtgaagacgcgtacaaacaggttttaagactgagtagaaggttgattaatggggatcttgaacatcagtggtttgaagagagtttgcggcaagcttacaataacaaagcgtttaaactctatacaattgataaagtcacccaatcgttggtaaagcatgctcataccttgatgactgacgctaaaactgcggaaataatggctttgttcgttaaagatagaaatgcctccaccacgagtgcgaaggaccaaattatctatcgcttgcaggtgcgctcacatatgtccaacacagaaaatatgtttagaatagagtttgataaaagaactctgcatgtttccattcaatatattgcacttgatgatttgacactaaaggaaccaaaggcagacgaagataaatggaaatattatgtaacatcgtatgctctgccacatccaacagaaggcattccgcacgagaaactgaagataccatttttggaaaggctcatcgaatttggacaagatattgatggaacagaggtagatgaggagttttcccccgaaggcatctctgtatcgacattgaaaattaagatccagcctataacctaccaactgcatattgaaaacgggtcttacgatgtttttacccgtaaggctactaacaaatatcctactattgctaatgataatactcaaaaaggaatggtttctcagaagaaggagttgatatcaaaatttctagactgtgcagttggtttgagaaataatttagatgaggctcaaaaattaagtatgcaaaaaaaatgggagaacctaaaagacagcatagcaaagacgagtgctggaaaccagggaatagagagtgaaaccgaaaaaggtaaaattacgaaacaggagcagtcggataatttggacagctctactgcaagtgtactgcctgcatccattactactgtaccacaagatgataatatagaaacgactgggaatactgaatcttcagacaagggggctaagattcaataa

SEQ ID NO: 221 - DNA, 3246 bp, Saccharomyces cerevisiae
atgtctgctgctcctgtccaagacaaagacactctgtccaatgccgagcgtgcgaagaacgtcaacgggttgcttcaggtgctcatggacattaacactctgaacggagggagctccgacactgctgataagataagaattcatgccaaaaacttcgaggcagctttgttcgcaaagagctcttcaaagaaagaatacatggacagcatgaacgaaaaagttgctgtcatgcgcaacacgtacaatacgaggaaaaacgccgttactgctgctgccgctaataacaacattaaacccgtggaacagcaccatattaacaacttgaaaaattctggcaacagcgccaataatatgaatgtcaatatgaatctaaacccacagatgttcttgaatcagcaggctcaggcaaggcaacaggttgcgcaacaattaagaaatcaacaacaacaacaacaacagcagcagcagcaacagaggcgtcaattgactcctcaacaacaacaattagtgaaccagatgaaagtggcacctattcccaaacaattactgcaaagaattcctaacattccacccaatatcaacacctggcagcaggtcactgctttggctcaacaaaagctattgacacctcaggatatggaagctgcgaaggaagtctacaagattcaccagcagttgctattcaaagcaaggctacagcaacaacaagcacaggctcaagcacaagctaataacaacaacaacggcctcccccaaaatggtaatattaacaataacataaatattcctcaacagcagcaaatgcaacctcccaattcaagtgcgaacaacaaccctttgcaacagcaatcatcacaaaataccgtaccaaacgtcctcaaccaaattaaccaaatcttttctccagaggagcaacgcagcttattacaagaagccatcgaaacctgcaagaattttgaaaaaacacaattgggtagtacgatgacggaacctgtcaagcaaagttttattaggaaatacattaaccaaaaggccctgagaaaaatccaagctttgagagatgttaagaacaacaataacgctaacaacaacggctcgaaccttcagagagcccaaaatgtccctatgaatatcatccaacaacaacaacaacagaacacgaacaataatgacaccattgccacttctgctacacctaatgctgccgctttctctcagcaacagaacgcaagttctaaattatatcagatgcaacaacagcaacaagctcaagctcaagctcaagctcaagctcaggcacaggctcaagcacaagctcaagcacaggcggcacaagcggcgcaagcgcaagcacaagcacaagcacaagcacaagcacaggcacaggcacaggcacaagcccaggcgcaggcccaagcccaagcccaagcacaagcacaagcacacgctcagcaccagccctcccaacaaccacaacaagctcagcaacaacctaacccactacatgggttgacacctactgcaaaggatgtcgaagtaattaagcaattgtccttggatgcttctaagaccaacctaaggcttacggacgtaacaaattctttatccaatgaagaaaaggaaaaaattaaaatgaagttaaagcaaggtcaaaagctttttgttcaggtgagtaatttcgccccacaagtctacatcatcacaaagaatgaaaacttcttgaaggaagtttttcagttaagaatctttgtaaaagagatcctagaaaaatgtgccgagggtatatttgttgttaaattagacaccgttgacaggttaattattaagtatcaaaaatattgggaaagtatgagaattcaaattttaagaagacaagctattttaagacaacaacagcaaatggcaaacaacaatgggaacccaggcactacttctactggaaacaataataatattgcaactcagcaaaatatgcaacagtcactacagcaaatgcagcatttacagcaattgaaaatgcagcagcaacaacaacagcagcaacaacaacagcagcaacaacaacagcagcaacaacaacagcaacagcacatatatccctcctcgactcctggtgtggctaattattcggcaatggctaatgcacccggtaacaatatcccatatatgaaccataaaaatacctctagcatggattttttgaactctatggaaaatacaccaaaagttcccgtatccgctgcggcaaccccatcactgaacaagacgatcaacggtaaggtgaatggcaggacaaaatctaatacgatacctgttaccagcattccatcaacaaataagaaactatcaatttcaaatgccgctagtcaacaacccactcctaggtctgcatcaaataccgctaagtcaaccccaaatacaaatccttctccactgaaaacccaaactaaaaatggcacaccaaaccccaataatatgaagactgtacagtctcctatgggtgcacaaccatcatataatagtgccattatagagaatgcattcagaaaggaagaactcttgttaaaagatttggaaatcaggaagttggagatatcttctcgttttaaacatcgccaagaaattttcaaagattctcctatggatttgtttatgagtacgctgggtgattgcttaggtatcaaagatgaagagatgcttacgtcatgcactatcccaaaggctgtggttgatcacatcaacggctctggcaaaaggaagcctacaaaagcggctcaaagggctcgcgatcaagattccattgacatttccataaaagacaacaaattggttatgaagagtaaattcaataagagcaataggtcgtattcgatagcgttgtccaatgtagctgctatattcaagggtatcggtggtaactttaaagacttgtccactttggttcattcatcatcgccgtccacatcttctaatatggatgtcggcaacccaagaaaaagaaaagccagcgtattagaaataagcccgcaagattcgatagcctcggtgctatcaccagattcaaatataatgagtgattctaaaaaaattaaagtagattctcctgatgacccattcatgacaaaatcaggagccacaactagtgaaaaacaagaagtaacaaatgaagctccatttttaacttctgggactagttcagaacaattcaatgtatgggattggaataattggacaagtgctacttga

SEQ ID NO: 222 - DNA, 1512 bp, Saccharomyces cerevisiae
atgaacatattgctaatgcaacggatagttagttttattttggtggttagtcaagggcgatactttcatgttggagagctgaccatgacaatgttgaaaagacctcaagaagaagagtcagataataatgctacgaagaagctgaaaacgagattaacgtacccttgtatcctcgggaaggataaggtgacgggcaaattcatctttcctgcaatcactaaagatgatgttatgaatgcaagactgtttttgaaggacaacgatttgaaaacttttttagaatattttttgccagtggaagtcaactcgatctacatttatttcatgataaaattgctgggattcgacgtcaaagataaagaactatttatggcgttaaactctaatatcacttctaataaagaacgttctagtgcggaactatcgtctatacatgcaaaagctgaagatgaggacgagttgaccgatcccttagagaaaaagcatgcagtaaaattaatcaaagatttacaaaaagccataaataaagtccttagtacaaggcttcgattacccaacttcaatacgattgatcatttcaccgccactttgcgtaacgccaaaaaaattttggttctgacgggcgctggagtttccacttctttaggtattcccgattttagatcatctgaggggttttattctaagatccgacaccttggattggaagaccctcaggatgttttcaacttggatatattcttacaggacccttctgttttttacaatattgcccatatggttttaccaccagagaacatgtattctccgttgcacagcttcattaaaatgctacaagacaagggcaaactattaagaaactacacacagaatatagataacttagaatcgtatgcagggatagatcctgataaattagtgcaatgtcacggttcattcgccactgcatcttgtgtgacttgccattggcaaatacctggtgagaaaatattcgaaaatattagaaaccttgaactaccattatgtccctactgttatcaaaaaagaaaacaatattttcctatgagtaatggcaataatacggtgcaaacaaacataaatttcaactcacccattttaaagtcgtatggtgttttgaaaccagacatgacattttttggcgaggccctgccatcgcgatttcataagaccatccgcaaagatatactggaatgcgatctattgatttgtataggaacaagtttgaaagtggctcctgtgtctgaaattgtcaatatggtaccttcacatgttccgcagattttgatcaatagggatatggttacacatgcagagtttgacttaaacctgttgggattctgtgatgacgtggccagtctcgtagctaaaaaatgccattgggatataccgcataaaaaatggcaagatctaaagaagatagattacaactgtacggaaatagataaaggtacgtataagattaagaaacagccacgaaagaaacaacagtaa

SEQ ID NO: 223 - DNA, 1047 bp, Saccharomyces cerevisiae
atgtctatcccagaaactcaaaaaggtgttatcttctacgaatcccacggtaagttggaatacaaagatattccagttccaaagccaaaggccaacgaattgttgatcaacgttaaatactctggtgtctgtcacactgacttgcacgcttggcacggtgactggccattgccagttaagctaccattagtcggtggtcacgaaggtgccggtgtcgttgtcggcatgggtgaaaacgttaagggctggaagatcggtgactacgccggtatcaaatggttgaacggttcttgtatggcctgtgaatactgtgaattgggtaacgaatccaactgtcctcacgctgacttgtctggttacacccacgacggttctttccaacaatacgctaccgctgacgctgttcaagccgctcacattcctcaaggtaccgacttggcccaagtcgcccccatcttgtgtgctggtatcaccgtctacaaggctttgaagtctgctaacttgatggccggtcactgggttgctatctccggtgctgctggtggtctaggttctttggctgttcaatacgccaaggctatgggttacagagtcttgggtattgacggtggtgaaggtaaggaagaattattcagatccatcggtggtgaagtcttcattgacttcactaaggaaaaggacattgtcggtgctgttctaaaggccactgacggtggtgctcacggtgtcatcaacgtttccgtttccgaagccgctattgaagcttctaccagatacgttagagctaacggtaccaccgttttggtcggtatgccagctggtgccaagtgttgttctgatgtcttcaaccaagtcgtcaagtccatctctattgttggttcttacgtcggtaacagagctgacaccagagaagctttggacttcttcgccagaggtttggtcaagtctccaatcaaggttgtcggcttgtctaccttgccagaaatttacgaaaagatggaaaagggtcaaatcgttggtagatacgttgttgacacttctaaataa

SEQ ID NO: 224 - DNA, 1815 bp, Saccharomyces cerevisiae
atgagtgccaatagcccgacaggaaacgatccccatgtatttggtattcctgtgaacgcaacaccatccaatatgggttcgccaggcagtccagttaatgtaccacctcctatgaacccagcggtagcaaatgtaaatcatcctgttatgaggacaaacagtaatagtaatgccaatgaaggtactaggactttaaccagggagcaaatacagcaattgcagcaaagacaacgattattactacagcaaagactactcgaacaacagagaaaacagcaagcactccaaaactatgaggctcagttttatcaaatgcttatgaccttaaacaaaagacctaaaagactttacaattttgtggaagatgcagactcaattttgaaaaaatatgagcaatatttacacagttttgaatttcatatttatgagaataattataagatttgcgctcctgcaaatagcaggttacaacagcagcaaaagcaacccgagctgactagtgacggtttaatattaaccaaaaataacgaaactttgaaagaatttttagagtacgtcgcaagaggaaggattcctgatgctattatggaagtcttaagggactgtaatatccaattttacgaaggaaatcttattttacaggtttatgaccatactaatacagttgatgtcacccccaaagagaacaagcctaatttgaacagctcttcttccccttcaaataataatagcacacaggacaattccaagattcaacaaccatccgaacctaatagtggtgtggcaaatacaggcgcaaacacagcaaataagaaggcatccttcaaacgacctagagtatatcgaacattgttgaagcctaatgatttgacgacgtattatgacatgatgtcttatgcagataatgccaggttttctgatagcatttatcaacagtttgaatctgaaattttaacacttaccaagaggaacttgtcactaagtgtgccattaaatccatatgagcaccgagacatgttggaggaaacagctttttcagaacctcattgggatagtgaaaaaaaatccttcattcacgaacatcgtgctgagtctacgagagaaggcacgaagggcgttgtagggcatatcgaagaacgcgatgaatttccgcaacatagttcaaattatgaacagttgatgttgatcatgaatgaacgcacaacaacgataaccaactccacgtttgctgtttcgttaaccaagaatgcgatggagattgctagctccagttccaacggggtacgtggagcttcgtcctcaacttcgaattccgcatcaaatacaaggaataacagtttggctaatggcaatcaagtagctttggcagcggcagcggctgcggctgcagtgggatctaccatgggtaacgataataatcaatttagcaggctgaaatttattgaacaatggagaataaataaagaaaaaagaaaacagcaggctttaagtgcaaacatcaatccaacccccttcaacgctagaatatcgatgacagcaccattgactccacagcaacagctacttcaaagacaacagcaggccttggagcaacaacagaatggtggagccatgaaaaacgcaaataaacggagtggtaacaatgccaccagtaacaataataataataataataatttagataaaccaaaggttaagcgacctagaaaaaatgcaaaaaaaagcgagagcggtacaccagctcctaaaaagaaaagaatgactaagaagaaacagagcgcaagtagcacgccctcttctactacaatgtcataa

SEQ ID NO: 225 - DNA, 1026 bp, Saccharomyces cerevisiae
atgcagatagtacaaattgagcaggccccaaaagactacataagcgacatcaaaataatcccttccaagtcactgcttttgattacgtcttgggatggctctttaacagtctacaaattcgacattcaagcaaagaatgttgaccttttacaatcgctacgatataaacatccgttattgtgctgcaatttcatcgacaataccgatctgcaaatatacgtgggaactgtacagggtgaaattctaaaagttgatttgataggtagtcccagcttccaagctttgacgaacaatgaagccaatttgggtatttgccgaatatgcaaatatggagacgataaactcattgccgcgtcatgggatggcctgatagaggttatcgaccctcgcaattatggtgatggagttattgctgttaaaaatttgaactctaataacacaaaggtgaagaataagatatttactatggatacaaactcctctcgattgatcgttggtatgaacaatagtcaggttcaatggtttcgcctgccactctgtgaggatgataacggaacaattgaagaatcaggactgaagtaccaaataagagatgtcgctcttttaccgaaagaacaagaaggttatgcatgtagcagcattgacgggcgagttgctgtggagtttttcgatgatcagggcgatgattacaactcaagcaaaagatttgcatttagatgccaccgtttgaatttaaaagatacaaacttagcgtatccagtaaattctattgaattttccccccgtcataagttcctatacacggctggctctgatggcataatttcatgctggaacctacaaacccgcaagaaaataaaaaatttcgccaaatttaacgaagacagcgtggttaaaattgcttgttcggacaatattctatgtctggcaacttctgatgatactttcaagacaaacgccgcaattgaccaaactattgaactaaacgcaagttcaatatacataatatttgactatgagaactga

SEQ ID NO: 226 - DNA, 2370 bp, Saccharomyces cerevisiae
atgccactgtgtgagaaagggaatgatccaatcgatagctctactattgatagcctctgcgctgcatttgacaagactttgaaatccacccctgatgtccaaaaatacaatgatgctataaatacaattttccagctaaggcaaaaatctgaatctgggaaaatgccggctgatttaacgaatagtgaagcccttaaagatcgtcagaaaatcgaagaaatattgaccaggtcttaccaagaccattctgagtcccgcgtacatttatctaaacttatacagaatgatattcccttcgcactcaatttatttgaaatattatcaagatcatccatccacgttttcgttggttgcttttccaataaagacgcaacaattgcattgttaaatgagctgcaaattaggatacactatggcgaggacacccacgtcacgtatttactgagcataattctacaactattgaataagttcaagtacaatttcaaggaggtgcgatttttagtgaaggaacttattttgcgcataagcgaagatgaagtaaaatcgatgatgcttataatttttgctgaattacaatcaagcttccaaaaagactttgataaagcagtggtcgatttcatgagtagcttaattgtggaggcagaaatagacgttggaaatgatccattgtccatcatagtcaaaaccctatcagaattatatccttcattgacaacactttgttctgaaatatttttgaccaaaggactaagtaagctttttaagaagagagtattcgaagagcaggatttacagtttacaaaagaactgctgcggttattatcttccgcgtgtattgatgagaccatgagaacgtatattacagaaaattaccttcaactattggaaagatcactaaacgtggaagatgtacaaatatattctgcattggtgttagttaagacatggtctttcacaaaattaacgtgtataaacttgaagcaactgtccgaaatctttataaacgctatttcaagacgtatcatgccaaaaattgaaaatgtcaatgaaagtgcggttaagctcgaggaggttcctaaagttgaaatgtccgttgaagcgctggcatatttaagtctaaaggcctcggttaagattatgattaggagtaacgagagctttactgaaattctattaactatgattaaaagccagaaaatgacgcactgtttatatggtctcttagttatcatggcaaatttgtccactttaccagaggaatccaatggtagttcacagtctatcaatgacttaaaaaattacgcagatttgaagggcccaggcgcagacaaggtaggcgcagagaaagaatcaaaggaagatattcttttgtttaacgaaaagtacatattaaggaccgagctgatttcctttttgaagagggaaatgcacaatttgagccccaattgtaaacaacaagtggttagggttatatataacataacccgctcgaaaaacttcattcctcaatgcatttcacaaggtggtacaactatcatcttagaatatttggccaataagcaggacataggggagccaatacggattctagggtgtcgggcattaacaagaatgctaatattcactaatccgggtttgatatttaaaaaatactctgcattaaacgctatccccttcttgttcgaattgttgccaaggtctacgccagtggatgacaatccgttacataacgacgaacaaataaagctgacagacaattacgaggcactattagctctgacgaacttggcatcatcggagacctctgatggtgaagaagtttgcaagcatatcgtatctacaaaagtatattggtccacgattgagaatctgatgctagacgaaaacgttcctttgcaaagatcaacattagagctcataagtaatatgatgagtcatccattaacgattgctgctaagttctttaacttggaaaatcctcagagtttgagaaattttaatatattggtgaaacttcttcagttatccgacgttgagtctcaacgagcagtggctgctatctttgctaacattgccaccacgattccattgatagctaaggaactgttaaccaagaaagaactaatcgaaaatgctattcaggtatttgcggatcaaattgatgacattgagttaagacaaaggttacttatgcttttttttggattattcgaagtgataccggataatggcactaacgaggtgtatccgttgctgcaagaaaatcaaaaattaaaggatgctttaaatatgtctttaaagagaggcgattcaggaccagagttttcagcggcaattcccgttatccttgctaaaattaaagtctaa

SEQ ID NO: 227 - DNA, 867 bp, Saccharomyces cerevisiae
atgtcggaagacgaattttttggtggtgataatgaagccgtttggaacggttccagattcagtgattcacctgagttccaaacgttgaaagaagaggttgctgcagagttatttgaaataaatgggcaaataagcacgctgcagcagtttaccgcgacacttaagtcatttatagatcggggagatgttagtgcgaaagttgtggaaagaattaataagagatctgtggcaaagatagaagaaataggcgggctcattaaaaaggtcaatacatcagtgaaaaagatggatgcgattgaggaagctagcctggataagactcaaataatagcgagagagaaacttgtgagggatgtcagttactcttttcaagagtttcaaggtattcagcggcagtttacccaagtaatgaaacaagttaatgaaagagcaaaagaatctcttgaagcaagtgagatggcaaatgatgctgctttattagatgaagaacaaaggcagaatagctcaaaaagtactcgaataccaggcagccaaatagtcattgagagagacccgataaataacgaagagtttgcttatcagcaaaatcttatcgagcaaagagaccaggaaatcagcaatattgaaagaggtataacggaactgaacgaagtttttaaagatttgggaagtgttgttcaacagcagggtgtgctagttgacaatattgaagcaaatatttatacaacgtcagataacactcaattggcttcagacgagctaaggaaggccatgcggtaccaaaaacgtacgagcagatggagggtgtatttgttgattgtgcttctcgtaatgcttctttttatttttctcattatgaaattgtaa

SEQ ID NO: 228 - DNA, 3819 bp, Saccharomyces cerevisiae
atgccgaacgaagataatgaacttcaaaaagcaattgagaaccatcataatcaactactaaaccaggataaagaaaatgctgacagaaatgggtctgttatagaagacctcccattatacgggacaagtataaaccagcagtctacccctggagatgttgacgatggaaaacacttactgtatccagatattgccaccaacctaccactgaagacttctgacagacttttggacgatatactttgcgatactatttttctcaattctacagacccgaaggtcatgcaaaagggcctgcaatcgaggggtattttaaaagagtctatgctttcttactcaactttcagaagtagtattcgccctaactgcttgggttcattaactgatcaagtggtttttcaaacaaaatccgagtatgattccatttcatgcccaaaatataataaaatacatgtatttcaggcggtcatctttaatccatcactggcagaacagcaaatttcaacttttgatgatattgttaaaattcctatttatcatcttaaggttagcgtaaaagtccgccaagaactggagcggttgaagaagcatgtcggtgttactcaattccactcactagatcatttgcacgaatacgatcgagtagacctttcgacttttgattcttccgatcctaatttgttggattacggtatttacgtttctgatgatactaacaaactgatcttgattgaaatttttaaacccgagtttaattcacctgaagagcatgagagttttactgccgacgcaattaagaagagatacaatgctatgtgtgtaaaaaatgaatcactagataaaagcgagacgccatctcaagttgactgtttttacacactttttaaaatttttaaagggcctttgacgaggaaaagtaaagcggaacctacaaagacaattgattctggaaatttggcccttaacactcacctgaatcctgaatggttaacgtccaagtatggatttcaagcaagctcagaaatcgatgaggaaactaatgagatatttactgaatacgtccctccagatatggtggactatgtaaacgatttggagacaagaaaaattcgagaatcgtttgtgaggaagtgtttacaactgatattttggggtcaactatctacctcattactggcacctaattctcccttgaaaaatacgaaaagcgtaaagggaatgtcttcattacaaacttctttctcaacactaccttggttccatttattgggagaatccagagcaaggattctattaaattccaatgagcaaactcattcgcctttggacgcagaacctcattttattaatctttccgtttcgcattattataccgatagagatataatcagaaactacgaatctttgtcttctttggatcctgaaaatattgggctgtattttgacgcactgacatacattgcaaataggaagggggcatatcaattgattgcttactgtggaaaacaggacattataggccaagaagctctagaaaatgctttgttaatgtttaaaattaaccctaaagagtgtaacatctccgaattaaatgaggcgactttgctatctatttacaaatatgaaacatcaaataagagccaagtaacctctaatcacctaacaaatttgaaaaatgctctaagattgttggccaaatataccaaatctgacaaactaaaattttacgtcgatcatgagccctacagagctttatcccaggcatacgacacactttcaattgacgagtctgttgatgaagacattataaaaactgcatattcggtcaagattaacgactctcccggattaaagttggattgtgatagagcactttacaccattgctatcagtaaaagaagccttgatttgttcaattttttaacagaggaatgcccacagttttccaactattatggtccagagaagcttgattaccaagaggcattgaagcttcttcaagtgaatgaaaatgcctctgacgaaaccattttgaaaatctttaaacaaaagtggtttgatgaaaacgtttatgagcctgaccaatttcttattttgagggcagcattgaccaaaatcagtatagaaagaaattcaactttaatcaccaacttcttactaactggtacgatagatccaaattccttgccgccagaaaattggccaactggcattaataatatcgggaacacctgttacctaaattctttattacaatattacttttccattgcgccactaagaagatatgtattggaatatcaaaaaacggtagaaaatttcaatgaccacctctctaatagtgggcatattagaagaattggtggaagagaaattagtagaggcgaagtggaaagatctattcaattcatataccaacttcgcaaccttttctatgcgatggttcatacaagagaaagatgtgtaacaccctcaaaagagctagcatatttggcatttgctccaagtaatgttgaagtagaatttgaagtggaaggcaataaagtagttgatcaaacaggagttctttcggattcaaagaaggaaacaacggatgacgcatttactacaaaaataaaggatacaagcctgattgatttagaaatggaagatggccttaatggcgatgttggtacagatgcgaacagaaaaaaaaatgaatcgaatgatgctgaagtaagtgagaacgaagatacaacaggattaacttcacctacgcgtgtggcaaaaatcagttctgatcaattagaaaatgctttggaaatgggtaggcaacaagatgttactgaatgcataggaaacgtgttatttcagatagaaagcggttcagagcctatccgatatgatgaagacaacgagcaatatgacttggttaagcaactattttatggtactactaaacaaagtattgttcctttgtccgcaacaaataaagtccgtacgaaagttgaaagattcctatcgttactgataaatattggcgatcatcctaaagatatttatgatgcgtttgattcttattttaaagacgaatatctgacaatggaagagtatggtgatgttatacgtaccgttgctgttacaacttttcctactattttgcaggtacaaatccaaagagtttattacgatcgtgaaagattaatgccgtttaaatccattgagcccttaccattcaaagaagttatttacatggacagatacgcggatacagagaaccctttattgttggcaaaaaagaaagaaacagaagaaatgaagcaaaagttgaaggtaatgaaaaatagacaaagagagcttttgagtcgtgatgattcagggcttacaaggaaggatgcatttttggagagtatcaagctattggaatcggataccataaagaaaactcctttaaaaattgaggctgctaatgatgtgataaagacgctgagaaacaacgttcaaaatatcgataatgaattgatgaaattatacaatgatatcaacagtttggaagagaaaataagccatcaatttgacgatttcaaggaatatggttactcactgttttcggtttttattcatcgcggcgaggccagttatggtcactattggatatatatcaaggacagaaatcgcaatggaatttggaggaagtacaatgatgaaaccatcagtgaggtccaggaagaggaggtcttcaatttcaatgagggtaacactgcaactccatatttcctagtatatgtcaaacaaggacaagaaggtgatattgagccattgaaaagaattctaaagtag

SEQ ID NO: 229 - DNA, 771 bp, Saccharomyces cerevisiae
atgtttaatttaattttatggcccttatttttgcttacctcagttgccattcctttgcagctgacattagaagtggtctatctgacgtcttccgttgacttctcaaaggcaagtgctgctaaaaccgctacttctttgggtcagtccccagttgtcatcaccatttataaatcactgttaaaatattggagtctttacgagttcatacattttatttacctctatactcctattgatgcctttctgaatttcttaccatttacctctttacttatgtcgtttggaagtatttgcttgacaagagagctggtatatgacttcattgcctttatggaatctcaaaacaagcttaccggattccttaataagataacagaaccgaattttaacagttatttactgttttcttcaatttataacatttggtttgcagatgacaccaatgacaaattcttattcggtaaattaactcagattttaatatctgtaacaaaaaggtacgaatttccaagaacattctacttagcaaaagtttctgatttcctacaaaatctcattctaacgagattgagaccctttgtaacggagcaaccccaaggcgacaaaaatcgctatcaaaacggtgaccgtgaatctacaaagaatggagctgcttatcagaagtcttcacaacaatcatcgtctttcgaacaaaattttaccagcactgagttcccgaatgattatgattttatggaggatattctagatgagacaactgaattggattag

SEQ ID NO: 230 - DNA, 5112 bp, Saccharomyces cerevisiae
atgaacataccacagcgtcaatttagcaacgaagaggtcaaccgctgctatttaagatggcagcatttgagaaatgaacacggaatgaacgctccgagtgtgccggaatttatctatttgactaaagtgttgcaatttgctgccaagcagcgacaagaattacaaatgcaacgccaacagcaaggcatttcaggttctcagcaaaatattgttccaaattcttcagatcaagcagaactgccgaataatgcttcctcgcacatttctgcttctgcttccccgcatttagctccgaatatgcaattgaatggaaatgagacctttagcacttctgctcatcaatcaccaatcatgcaaacacagatgccattaaatagcaatggtggtaataacatgctaccacaacggcaatcttcggttggttcattaaatgcaactaatttttcaccaacaccagcaaacaatggagaaaatgctgcggagaaaccagataactcaaaccataataacttaaaccttaataattccgagcttcaaccgcaaaatcgatcattacaagaacacaacatacaggattcaaatgtgatgccaggttcgcagataaattctcctatgccccagcaggcgcagatgcagcaggcacaattccaagcacaacaggcgcaacaagcgcaacaggcacaacaggcacaacaagcacaagcgagactgcaacaggggagaagattacccatgacaatgtttactgccgagcaatccgaactgttaaaggcccaaatcacatctctaaaatgtctagtaaatagaaagcctattccgtttgaatttcaggctgttatccaaaaatctattaaccatcctccagatttcaaaagaatgttactgtctttgagtgagtttgcgagaagaagacaacccacagatcaaaataaccaatcaaatctcaatggcggcaacaatacgcagcaacccggcaccaattcccactacaataacactaacactgacaacgtttctgggcttaccagaaatgcacctttagatagcaaagacgaaaattttgcatctgtttcaccagctggtccatctagtgtacataatgcgaagaatggcaccctcgataaaaattcgcaaactgtaagtggcactccaatcacgcaaacagaatcgaaaaaagaggaaaacgaaaccatttcaaacgtagctaaaacagctcctaatagtaataagacccacaccgagcaaaataacccgccaaaaccgcagaaacccgttcctttaaacgtccttcaagatcaatataaggaaggaataaaagtggtagatatagatgatcctgatatgatggttgattcttttacgatgcccaatatctcacatagtaatatcgattatcagactttattggccaattcggatcatgcaaagtttactattgaacctggcgttctaccggttggtattgacacacataccgctactgacatttaccaaactttaattgcattgaacctagatactacggtaaatgactgtttggataagctactaaatgacgaatgtacggaatcaaccagggaaaatgctctttacgattactacgccttacaattattaccgttacaaaaagccgtaagaggtcatgtattacaatttgaatggcatcaaaattctttattaaccaatacacatccaaattttctttcgaaaataagaaacatcaacgttcaagatgcattgctaacaaaccaactttataagaatcatgaattacttaagttagaaaggaagaagactgaggcagtggccagattaaagtcgatgaataaatccgccatcaatcaatataatagaagacaagataaaaagaataaaaggttgaagtttggccatagactcattgctacgcatactaatttagaaagggatgagcaaaagagagctgaaaaaaaggcaaaagaacgtttacaggctttaaaggcgaacgatgaggaagcctatataaaattattagaccaaaccaaagatacaagaattacacatcttctaaggcaaacaaacgcatttttagattctttaacaagagccgtgaaagatcaacaaaagtacactaaagaaatgattgattcccacataaaggaagcatcggaggaagtggatgatttgtccatggtccccaagatgaaggatgaagaatatgatgacgacgacgacaattctaacgtcgattattacaatgtcgcacatagaattaaagaagatatcaagaagcagccttcaatattggttggtggtaccttaaaggactatcaaatcaagggtttacagtggatggtttcgcttttcaataatcatttaaatggtattttagcagacgaaatgggtcttggtaagactatccagacgatatccttacttacatatttatatgaaatgaagaatattcgtgggccatatttagtcattgttcctttatctacactgtcaaattggagcagtgaattcgcgaaatgggctcctaccttaagaactatatcatttaaaggttcgccaaatgagaggaaagccaaacaggctaaaataagggcaggtgaatttgatgtagttctgactacctttgagtatattattaaagaaagagcacttttatccaaggtcaagtgggtccatatgattattgatgaaggccatagaatgaaaaatgcgcaatccaagctttctttaaccttgaacacacattaccatgcagattatagattaattttgacaggtacaccacttcaaaacaacttgccagaattatgggccttattgaattttgtgttacccaagattttcaattccgtgaaatcttttgatgaatggttcaatacaccctttgccaacaccggtgggcaagataaaattgagttgagtgaagaagagacgcttttggttatcaggagattgcataaagtcttgagaccctttttattgcgtcgtttgaaaaaggatgtagaaaaagaattaccggataaagttgagaaggtagtgaagtgcaaaatgagcgctttacaacaaattatgtaccaacaaatgttgaaatatcgtcgtctttttatcggtgatcaaaataataagaaaatggtaggtttaagaggtttcaataatcaaatcatgcaactaaagaaaatctgtaatcatccttttgtatttgaagaagttgaggaccaaatcaatccaacaagagaaacgaatgatgatatttggcgtgttgctggtaaatttgaactattagatagaattttgcctaagcttaaggcaactgggcatagagtattaattttttttcaaatgacccaaattatggatattatggaagattttttaaggtacattaatatcaagtacctgaggttagatggtcatacaaaatctgatgaacgtagtgagttgttacgtctatttaacgcccctgattctgaatacttgtgctttatcttatcaactagagcgggtggtttgggtttaaatttacaaactgcagacactgtcattatttttgatactgactggaatcctcatcaagatttgcaagctcaggatagagcgcatagaataggtcagaagaatgaagttagaattctaagattgattactacgaattcagttgaagaagttatcctagagagagcctacaagaagctagatatcgatggtaaagtcattcaagcaggtaaattcgataacaaatctacatcggaggaacaagaagcgttgcttagatctttgctggatgcagaagaggaacgtagaaagaaaagagagtctggtgttgaagaagaagaggagttaaaagactcagaaatcaacgaaattcttgctcgaaatgacgaagaaatggctgttcttacaaggatggatgaagatagatcaaagaaggaggaagaattgggagtaaagagccgtctcttagaaaagagtgaattacctgatatttacagtagagatattggtgccgagctcaaacgtgaagaatcagaatctgctgctgtttacaatggaaggggtgcaagagaacgtaagacggcaacttacaacgataatatgtctgaagaacagtggttaagacaatttgaagttagcgatgatgaaaaaaatgacaagcaggcaagaaaacaacgaacaaagaaggaagataagagcgaggcgattgatgggaatggagaaattaaaggcgaaaacattgacgctgataatgacggtcctagaattaataatatatcggccgaagatcgggcagatacggatttggcaatgaatgacgatgatttcttgtctaaaaagagaaaggcaggaaggccccgtggaagacctaagaaagtgaaacttgaaggatctgaaaattctgaaccaccagcgcttgaatcaagtccagttactggcgataactctcccagtgaggattttatggacattccaaaaccacgtacagcgggaaaaacatctgttaaatcagcaagaacttcaacaaggggccgtggtcgcggtcgtggtcgtggtcgagggcgaggacgtggacgaggaaggccgccaaaagcaagaaatgggcttgattatgtccgcacgcccgctgcggcaacgagtcctattgatattagagagaaagttgctaaacaagctctagacttataccactttgctctcaattatgaaaatgaagctggaaggaaactttctgacattttcctctccaaaccttccaaagcactatatcccgactactatatgattattaagtaccctgtcgcctttgataatattaacacgcatattgaaacgctagcttacaactcccttaaagagacccttcaagactttcatctgatattttccaatgctaggatatacaatactgagggttcagtagtatacgaagactctttagaactggaaaaagtggttacgaagaaatactgtgagataatgggcgataactcacaactcgactttactgaatttgatgaacagtatggcaccagacccctcgtcttgcctcctgttgttacttcaagcgtggctgaatctttcacagatgaagcggactcgagcatgacagaagcgagtgtatag

SEQ ID NO: 231 - DNA, 456 bp, Saccharomyces cerevisiae
atgtctgactgggatacaaatactattattggtagcagagcaagagctggtggttctggcccaagggccaacgttgccagatctcaaggccaaattaatgctgcaagaagacaggggttggttgtttccgttgacaagaagtacggatcaaccaatacgaggggtgacaacgagggtcagaggttgaccaaggtcgaccgtgagacagatattgtcaaacccaagaaattggatccaaatgtcggtagggccatctcgcgtgccagaacagacaagaaaatgtcgcagaaggatttggcaaccaaaattaacgaaaagccaaccgtggtaaacgattatgaagccgctagagctattccaaaccaacaagttttgtctaagttggaaagagccttaggcgttaaattgagaggtaacaacatcggttcgcctttgggagctccaaagaagaaatga

SEQ ID NO: 232 - DNA, 78 bp, Saccharomyces cerevisiae
atgtttagcttatcgaactctcaatacacctgccaagactacatatctgaccacatctggaaaactagctcccactaa

SEQ ID NO: 233 - DNA, 378 bp, Saccharomyces cerevisiae
atgctattttatcattgttcttcattttcctcatcttcttcatcttcttcatccagtgcttccactagaagactgccatttggccatagctggggcacttggtccaccgacatgtgctcgttaggatgcacagtatacttagggaagggcgataccaattcaaagtcgaattcaagtttaccacgttcgagatcaaatgaggtaagctcgttgttgatgatgtggccgagctcttcgatagtggtagcagtagcagtttcgagttgtccttctttaaagcagagcatatcgtcgtctgcagataattgcctgattttgtcctggagggcgttagaccattctgcggggtcactagaatataccgcgaggtatatgtga

(19) United States
(12) Patent Application Publication
(10) Pub. No.: US 2011/0061129 A1
(43) Pub. Date: Mar. 10, 2011

(54) Disease Resistant Transgenic Plants

(75) Inventors: Nilgun E. Tumer, Belle Mead, NJ (US); Mohamed Anwar Bin Umer, Paterson, NJ (US); John McLaughlin, Doylestown, PA (US)

(73) Assignee: RUTGERS, THE STATE UNIVERSITY, New Brunswick, NJ (US)

(21) Appl. No.: 12/919,613

(22) PCT Filed: Mar. 2, 2009
(86) PCT No.: PCT/US09/01337

§ 371 Date: Nov. 17, 2010

Related U.S. Application Data
(60) Provisional application No. 61/032,761, filed on Feb. 29, 2008.

(51) Int. Cl. C12N 15/87; A01H 5/00

(52) U.S. Cl. 800/279; 800/301

(57) ABSTRACT

Provided are transgenic plants, seed and plant parts which display increased resistance to fungal disease or mycotoxin. Also provided are methods of making transgenic plants with increased resistance to fungal disease or mycotoxin.

### CROSS REFERENCE

This application claims the benefit of the filing date of U.S. Provisional Patent Application No. 61/032,761 filed Feb. 29, 2008, the disclosure of which is hereby incorporated herein by reference.

### STATEMENT REGARDING FEDERALLY SPONSORED RESEARCH

The development of this invention was supported by USDA-ARS grant FY08-TU-100. Thus, the Government may have rights in the invention.

### TECHNICAL FIELD

This invention relates generally to agricultural biotechnology, and more specifically to transgenic plants, and methods and genetic elements for increased resistance to fungal diseases in plants.

### BACKGROUND ART

Fusarium Head Blight (FHB), caused mainly by the fungus Fusarium graminearum, results in yield reductions and in the contamination of grain with trichothecene mycotoxins, including deoxynivalenol (DON), diacetoxyscirpenol (DAS), T-2 toxin, and trichothecin (T-cin), among others. Kimura et al., Biosci Biotechnol Biochem 2007. Using trichodermin as a model compound, trichothecenes were shown to inhibit protein synthesis by targeting the peptidyltransferase center of eukaryotic ribosomes (Wei et al., Proc Natl Acad Sci USA, 1974.71:713-7), and the gene responsible for the increased trichodermin resistance (TCM1) was shown to encode ribosomal protein L3 (RPL3) in yeast (Fried et al., Proc Natl Acad Sci USA 1981, 78:238-42). Subsequent studies provided evidence that L3 is targeted by other trichothecenes, including DON in yeast (Fernandez-Lobato et al., Biochem J 1990, 267:709-13) and in plants (Di et al., Mol Plant Microbe Interact 2005, 18:762-70; Harris et al., Molecular Plant Pathol. 2001, 58:173-81; and Mitterbauer, et al., Plant Biotechnology J. 2004, 2:329-340). Trichothecenes have been reported to have diverse roles in the cell that are not limited to the inhibition of protein synthesis. These include single stranded breaks in DNA (Lafarge-Frayssinet et al, Mutat. Res. 1981, 88:115-23), inhibition of mitochondrial electron transport and chloroplast activity, membrane damage (Khachatourians, G. G., Can J Physiol Pharmacol 199068:1004-8), and inhibition of the cell cycle (Danuta, et al., Caryologia 2005, 58:86-930. It is not clear if these effects are cellular responses to protein synthesis inhibition or if trichothecenes have multiple modes of action. There is evidence that DON acts as a virulence factor during Fusarium infection (Proctor et al., Mol Plant Microbe Interact 1995, 8:593-601). However, the molecular mechanisms that control mycotoxin sensitivity in wheat or barley are not well understood and the genes targeted by DON other than ribosomal protein L3 have not been identified.

### SUMMARY OF THE INVENTION

The invention is directed to methods for making transgenic plants and transgenic plants and plant parts themselves which display increased resistance to a fungal disease or mycotoxin over non-transgenic control plants. The transgenic plants contained altered expression levels of a gene which confers resistance or sensitivity to a fungal disease or mycotoxin.

### BRIEF DESCRIPTION OF DRAWINGS

FIG. 1 is a series of pie charts showing the YKO strains identified as resistant to T-cin as classified by their associated roles in the cell. Charts represent the distribution of the types of genetic expression associated with the deleted genes that showed resistance to 4 μM of T-cin (A), 6 μM of T-cin (B) and 8 μM of T-cin (C), respectively.

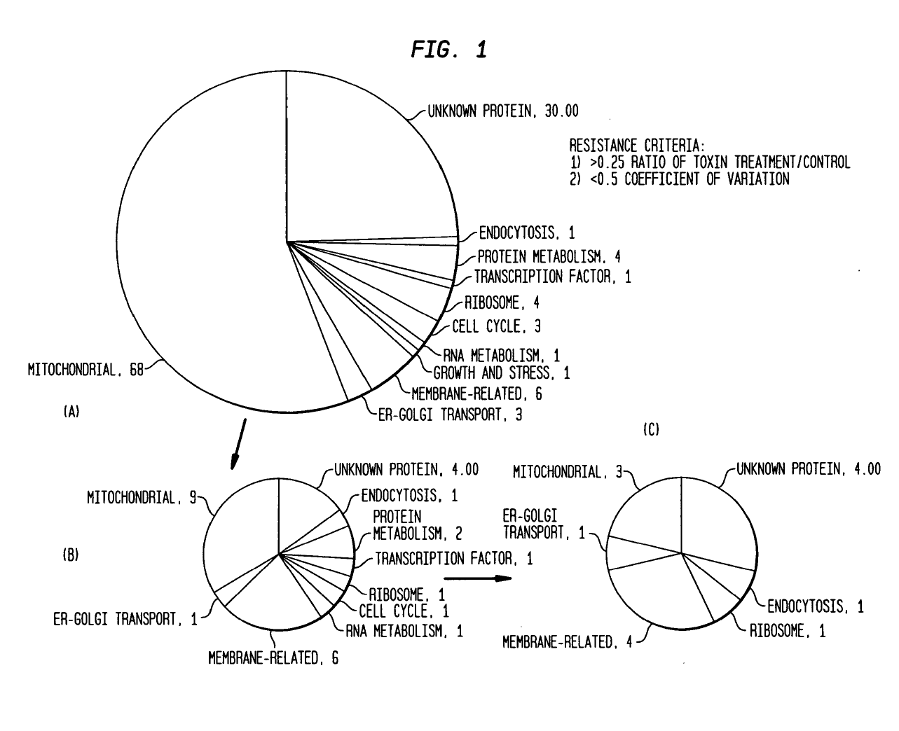

FIG. 2 is pie chart showing the YKO strains identified as sensitive to T-cin as classified by their associated roles in the cell.

FIG. 3 is a graph showing the results of the total translation assay.

FIG. 4 is a graph showing the results of the mitochondrial translation assay.

FIG. 5 is a graph showing the results of the both the total and mitochondrial translation assays.

### DETAILED DESCRIPTION OF THE INVENTION

The invention is directed to transgenic plants that display increased resistance to a fungal disease or mycotoxin. The invention also provides plant parts, e.g., leaves, stems and shoots, as well as plant cells and protoplasts which are used to produce the transgenic plant and/or produced by the transgenic plant. Non-naturally occurring seed derived from the transgenic plants is also provided. Methods of making the transgenic plants are further provided.

The transgenic plants or plant parts of the invention display altered expression of one or more genes, wherein the altered expression results in increased resistance to a fungal disease or mycotoxin as compared to a control plant without the altered expression.

In certain embodiments, the transgenic plant displays reduced expression levels of a gene which confers sensitivity to a fungal disease or mycotoxin. Non-limiting examples of genes which confer sensitivity to a fungal disease or mycotoxin (e.g., a sensitivity gene) are listed in Table 1 and include YGL139W, YDL054C, YDR157W, YDL056W, YGL135W, YKR010C, YLR099C, YLR193C, YGL110C, YKR012C, YDL045W-A, YLR262C-A, YDR533C, YPR047W, YMR187C, YNL011C, YKR046C, YER017C, YLR260W, YGR241C, YDL044C, YMR310C, YOL101C, YPR100W, YHR038W, YDR519W, YPL219W, YJL119C, YOL095C, YGL064C, YFR019W, YDR529C, YDR065W, YGR283C, YJL152W, YGL119W, YHR051W, YMR064W, YDL173W, YBR268W, YHR091C, YDR296W, YDR519W, YKR006C, YBR122C, YBR179C, YGR101W, YBR126C, YLR312W-A, YKL003C, YLR270W, YLR199C, YFR032C-A, YPL271W, YPL104W, YDR509W, YNL284C, YNL005C, YDR377W, YMR267W, YPR116W, YCR003W, YCR071C, YOL009C, YER058W, YGR219W, YCR046C, YDR462W, YPL052W, YJR101W, YPR166C, YER061C, YGL218W, YBR251W, YBR282W, YJL102W, YGL064C, YNL073W, YOR201C, YPL118W, YLL033W, YMR287C, YML110C, YLR439W, YDL044C, YNL081C, YER050C, YKL134C, YER087W, YJL193W, YLL006W, YDL056W, YBL090W, YOR241W, YLR295C, YBR120C, YCR024C, YMR293C, YBR163W, YPL173W, YPL005W, YBR104W, YER122C, YGL246C, YLR139C, YGR215W, YJL132W, YHR147C, YAL010C, YPL057C, YOL023W, YBR162W-A, YOR205C, YDL067C, YDL050C, YJL189W, YCR028C-A, YGR220C, YBR101C, YJL208C, YBR144C, and YGL251C.

Reduced expression includes any expression level of the sensitivity gene which is lower as compared to a control plant without altered expression and which results in increased resistance to a fungal disease or mycotoxin as compared to control plant. In certain embodiments, the sensitivity gene is not expressed at all.

In other embodiments, the transgenic plant displays increased expression levels of a gene which confers resistance to a fungal disease or mycotoxin. Non-limiting examples of genes which confer resistance to a fungal disease or mycotoxin (e.g., a resistance gene) include those listed in Table 6 and include YAL002W, YAL013W, YAL021C, YAL047C, YBL058W, YBL071W-A, YBL093C, YBR023C, YBR081C, YBR112C, YBR267W, YBR279W, YBR289W, YCR020W-B, YCR044C, YCR094W, YDL013W, YDL023C, YDL075W, YDL106C, YDR049W, YDR264C, YDR266C, YDR433W, YDR442W, YDR495C, YDR532C, YEL044W, YER070W, YER087W, YER151C, YFR010W, YGL012W, YGL038C, YPL050C, YPL084W, YPL129W, YPL254W, YPL268W, YGL058W, YGL070C, YGR006W, YGR063C, YGR092W, YGR104C, YGR196C, YGR262C, YGR271W, YHL025W, YHR013C, YHR030C, YJL028W, YJL095W, YJL129C, YJL175W, YJL184W, YJR090C, YJR102C, YKL002W, YKL139W, YKL213C, YKR023W, YKR082W, YKR093W, YLR025W, YLR027C, YLR226W, YLR234W, YLR240W, YLR322W, YLR330W, YLR337C, YLR338W, YPR072W, YPR173C, YPL042C, YPL045W, YKR004C-A, YLR370C, YLR417W, YML008C, YML062C, YML094W, YML112W, YMR032W, YMR077C, YMR202W, YMR263W, YNL054W, YNL064C, YNL084C, YNL097C, YNL133C, YNL140C, YNL147W, YNL229C, YNL236W, YNR051C, YNR052C, YOL004W, YOL051W, YOL068C, YOL086C, YOL148C, YOR026W, YOR035C, YOR036W, YOR124C, YOR152C, YOR290C, YOR298C-A, YOR302W, and YML013C-A.

The sequences of the specific sensitivity and resistance genes are available online at the Saccharomyces genome database (SGD) at http://www.yeastgenome.org and disclosed herein.

In certain embodiments, the transgenic plants of the invention display increased resistance to one or more diseases caused by plant fungi, including those caused by Fusarium (causing root rot of bean, dry rot of potatoes, head blight (scab) in wheat), Pythium (one of the causes of seed rot, seedling damping off and root rot), Phytophthora (the cause of late blight of potato and of root rots, and blights of many other plants), Bremia, Peronospora, Plasmopara, Pseudoperonospora and Sclerospora (causing downy mildews), Erysiphe graminis (causing powdery mildew of cereals and grasses), Verticillium (causing vascular wilts of vegetables, flowers, crop plants and trees), Rhizoctonia (causing damping off disease of many plants and brown patch disease of turfgrasses), Cochliobolus (causing root and foot rot, and also blight of cereals and grasses), Giberella (causing seedling blight and foot or stalk rot of corn and small grains), Gaeumannomyces (causing the take-all and whiteheads disease of cereals), Schlerotinia (causing crown rots and blights of flowers and vegetables and dollar spot disease of turfgrasses), Puccinia (causing the stem rust of wheat and other small grains), Ustilago (causing corn smut), Magnaporthae (causing summer patch of turfgrasses), and Schlerotium (causing southern blight of turfgrasses). Other important fungal diseases include those caused by Cercospora, Septoria, Mycosphoerella, Glomerella, Colletotrichum, Helminthosporium, Alterneria, Botrytis, Cladosporium and Aspergillus.

In certain embodiments, the transgenic plants of the invention are resistant to one or more mycotoxins. Mycotoxins include fumonisin, aflatoxin, ocratoxin, cercosporin, alternaria toxins, HC toxin and trichothecenes. Trichothecenes are a class of toxic, sesquiterpenoid secondary metabolites that are produced mainly by plant pathogenic fungi (Fernandez-Lobato et al., Biochem. J. 267:709-713 (1990). Trichothecenes include trichodermin, dexoynivalenol (DON), diacetoxyscirpenol (DAS), zearalenone, nivalenol, 3-acetyldeoxynivalenol, 15-acetyldeoxynivalenol, fusarenon-X, T-2 toxin and trichotecin (T-cin). Fusarium graminearum and F. culmorum produce DON, which contaminates a substantial portion of agricultural crops such as wheat, barley, oats, and maize.

The transgenic plants include a variety of higher plants including monocots (e.g., cereal crops) and dicots. Specific examples include maize, tomato, turfgrass, asparagus, papaya, sunflower, corn, rye, beans, ginger, lotus, bamboo, potato, rice, peanut, barley, malt, wheat, alfalfa, soybean, oat, eggplant, squash, onion, broccoli, sugarcane, sugar beet, beets, apples, oranges, grapefruit, pear, plum, peach, pineapple, grape, rose, carnation, daisy, tulip, Douglas fir, cedar, white pine, scotch pine, spruce, peas, cotton, flax, canola, ornamentals and coffee.

The transgenic plants may be produced in accordance with standard transformation techniques for the plant type of interest. See U.S. Pat. No. 5,675,322, Horsch et al., Science 227:1229-1231 (1985); and Hartman et al., Bio/technology 12:919-923 (1994).

The invention also includes plant parts (such as leaves, stems, and roots) which display altered expression of one or more genes as described herein resulting in increased resistance to fungal disease or mycotoxin. Also included are protoplasts which include the exogenous nucleic acids or altered genes as described herein. Further provided are non-naturally occurring seed produced by the transgenic plants described herein.

In certain embodiments, a protein encoded by a resistance gene described herein is applied directly onto plants. In certain other embodiments, a protein which interferes with the expression of a sensitivity gene described herein is applied directly onto plants. The plants may be transgenic, or non-transgenic.

The invention is also directed to methods of making a transgenic plant which display increased resistance to a fungal disease or mycotoxin. In certain embodiments, the transgenic plant has a genome which contains an exogenous nucleic acid comprising a transgene. The transgene may encode a protein which confers resistance to a fungal disease or mycotoxin, or it may encode a protein which interferes with the expression of a gene which confers sensitivity to a fungal disease or mycotoxin. In other embodiments, the transgenic plant has a genome which contains an alteration in a gene which confers sensitivity to a fungal disease or mycotoxin, wherein the alteration results in reduced expression of the gene.

The method of making a transgenic plant may comprise transforming a protoplast from a cell of a plant. The protoplast may be transformed with the exogenous nucleic acid (i.e., one encoding a resistance gene or one which encodes a protein which interferes with the expression of a sensitivity gene) or may be transformed by mutation of a sensitivity gene (e.g., through homologous recombination or the use of transposons). The transgenic plant may then be generated from the transformed protoplast. Alternatively, the method comprises introducing the exogenous nucleic acid or genome alteration into tissue of a plant to produce transformed plant tissue, and regenerating the transgenic plant from the transformed plant tissue.

Methods for preparing nucleic acids encoding are known in the art. In embodiments which include increased expression of a gene, the nucleic acid (e.g., a cDNA) is preferably inserted into a plant transformation vector in the form of an expression cassette containing all of the necessary elements for transformation of plant cells. The expression cassette typically contains, in proper reading frame, a promoter functional in plant cells, a 5′ non-translated leader sequence, the insert DNA, and a 3′ non-translated region functional in plants to cause the addition of polyadenylated nucleotides to the 3′ end of the RNA sequence.

Promoters functional in plant cells may be obtained from a variety of sources such as plants or plant DNA viruses. The selection of a promoter used in expression cassettes will determine the spatial and temporal expression pattern of the construction in the transgenic plant. Selected promoters may have constitutive activity and these include the CaMV 35S promoter, the actin promoter, and the ubiquitin promoter. Alternatively, they may be inducible e.g., wound-induced (Xu et al., Plant Mol. Biol. 22:573-588 (1993), Logemann et al., Plant Cell 1:151-158 (1989), Rohrmeier & Lehle, Plant Mol. Bio. 22:783-792 (1993), Firek et al. Plant Mol. Biol. 22:129-142 (1993), Warener et al., Plant J. 3:191-201 (1993)) and thus drive the expression of the resistance gene at the sites of wounding or pathogen infection. Other useful promoters are expressed in specific cell types (such as leaf epidermal cells, meosphyll cells, root cortex cells) or in specific tissues or organs (roots, leaves or flowers, for example). Patent Application WO 93/07278, for example, describes the isolation of the maize trpA gene that is preferentially expressed in pith cells. Hudspeth et al., Plant Mol. Biol. 12:579-589 (1989), have described a promoter derived from the maize gene encoding phosphoenolpyruvate carboxylase (PEPC) which directs expression in a leaf-specific manner. Alternatively, the selected promoter may drive expression of the gene under a light induced or other temporally regulated promoter. A further alternative is that the selected promoter be chemically regulated.

A variety of transcriptional cleavage and polyadenylation sites are available for use in expression cassettes. These are responsible for correct processing (formation) of the 3′ end of mRNAs. Appropriate transcriptional cleavage and polyadenylation sites functional in plants include the CaMV 35S cleavage and polyadenylation sites, the tml cleavage and polyadenylations sites, the nopaline synthase cleavage and polyadenylation sites, the pea rbcS E9 cleavage and polyadenylation sites. These can be used in both monocotyledons and dicotyledons.

Numerous sequences have been found to enhance gene expression from within the transcriptional unit and these sequences can be used in conjunction with the resistance genes of this invention to increase their expression in transgenic plants. Various intron sequences have been shown to enhance expression, particularly in monocotyledonous cells. For example, the introns of the maize Adh1 gene have been found to significantly enhance the expression of the wild-type gene under its cognate promoter when introduced into maize cells. Intron 1 was found to be particularly effective and enhanced expression in fusion constructs with the chloramphenicol acetyltransferase gene (Callis et al., Genes Develop 1:1183-1200 (1987)). In the same experimental system, the intron from the maize bronze-1 gene had a similar effect in enhancing expression (Callis et al., supra.). Intron sequences have been routinely incorporated into plant transformation vectors, typically within the non-translated leader.

A number of non-translated leader sequences derived from viruses are also known to enhance expression, and these are particularly effective in dicotyledonous cells. Specifically, leader sequences from Tobacco Mosaic Virus (TMV, the “Q-sequence”), Maize Chlorotic mottle Virus (MCMV), and Alfalfa Mosaic Virus (AMV) have been shown to be effective in enhancing expression (e.g. Gallie et al., Nucl. Acids Res. 15:8693-8711 (1987); Skuzeski et al., Plant Mol. Biol. 15:65-79 (1990)).

Numerous transformation vectors are available for plant transformation, and the genes of this invention can be used in conjunction with any such vectors. The selection of vector for use will depend upon the preferred transformation technique and the target species for transformation. For certain target species, different antibiotic or herbicide selection markers may be preferred. Selection markers used routinely in transformations include the nptII gene which confers resistance to kanamycin (Messing et al., Gene 19:259-268 (1982); Bevan et al., Nature 304:184-187 (1983)), the bar gene which confers resistance to the herbicide phosphinothricin (White et al., Nucl. Acids Res. 18:1062 (1990); Spencer et al., Theor. Appl. Genet. 79:625-631 (1990)), the hph gene which confers resistance to the antibiotic hygromycin, and the dhfr gene, which confers resistance to methotrexate. Vectors suitable for Agrobacterium transformation typically carry at least one T-DNA border sequence. These include vectors such as pBIN19 and pCIB200 (EP 0 332 104).

Transformation without the use of Agrobacterium tumefaciens circumvents the requirement for T-DNA sequences in the chosen transformation vector and consequently vectors lacking these sequences can be utilized in addition to vectors such as the ones described above which contain T-DNA sequences. Transformation techniques which do not rely on Agrobacterium include transformation via particle bombardment, protoplast uptake (e.g., PEG and electroporation) and microinjection. The choice of vector depends largely on the preferred selection for the species being transformed. For example, pCIB3064 is a pUC-derived vector suitable for the direct gene transfer technique in combination with selection by the herbicide basta (or phosphinothricin), described in WO 93/07278 and Koziel et al., Biotechnology 11:194-200 (1993).

An expression cassette containing the resistance gene DNA containing the various elements described above may be inserted into a plant transformation vector by standard recombinant DNA methods. Alternatively, some or all of the elements of the expression cassette may be present in the vector, and any remaining elements may be added to the vector as necessary.

Transformation techniques for dicotyledons are well known in the art and include Agrobacterium-based techniques and techniques which do not require Agrobacterium. Non-Agrobacterium techniques involve the uptake of exogenous genetic material directly by protoplasts or cells. This can be accomplished by PEG or electroporation mediated uptake, particle bombardment-mediated delivery, or microinjection. Examples of these techniques are described by Paszkowski et al., EMBO J. 3:2717-2722 (1984), Potrykis et al., Mol. Gen. Genet. 199:169-177 (1985), Reich et al., Biotechnology 4:1001-1004 (1986), and Klein et al., Nature 327:70-73 (1987). In each case the transformed cells are regenerated to whole plants using standard techniques.

Agrobacterium-mediated transformation is a preferred technique for transformation of dicotyledons because of its high efficiency of transformation and its broad utility with many different species. The many crop species which are routinely transformable by Agrobacterium include tobacco, tomato, sunflower, cotton, oilseed rape, potato, soybean, alfalfa and poplar (EP 0 317 511 (cotton), EP 0 249 432 (tomato), WO 87/07299 (Brassica), U.S. Pat. No. 4,795,855 (poplar)). Agrobacterium transformation typically involves the transfer of the binary vector carrying the foreign DNA of interest (e.g. pCIB200 or pCIB2001) to an appropriate Agrobacterium strain which may depend on the complement of vir genes carried by the host Agrobacterium strain either on a co-resident plasmid or chromosomally (e.g. strain CIB542 for pCIB200 (Uknes et al. Plant Cell 5:159-169 (1993)). The transfer of the recombinant binary vector, to Agrobacterium is accomplished by a triparental mating procedure using E. coli carrying the recombinant binary vector, a helper E. coli strain which carries a plasmid such as pRK2013 which is able to mobilize the recombinant binary vector to the target Agrobacterium strain. Alternatively, the recombinant binary vector can be transferred to Agrobacterium by DNA transformation (Hofgen & Willmitzer, Nucl. Acids Res. 16:9877 (1988)).

Transformation of the target plant species by recombinant Agrobacterium usually involves co-cultivation of the Agrobacterium with explants from the plant and follows protocols known in the art. Transformed tissue is regenerated on selectable medium carrying an antibiotic or herbicide resistance marker present between the binary plasmid T-DNA borders.

Preferred transformation techniques for monocots include direct gene transfer into protoplasts using PEG or electroporation techniques and particle bombardment into callus tissue. Transformation can be undertaken with a single DNA species or multiple DNA species (i.e., co-transformation) and both these techniques are suitable for use with this invention. Co-transformation may have the advantage of avoiding complex vector construction and of generating transgenic plants with unlinked loci for the gene of interest and the selectable marker, enabling the removal of the selectable marker in subsequent generations, should this be regarded desirable. However, a disadvantage of the use of co-transformation is the less than 100% frequency with which separate DNA species are integrated into the genome (Schocher et al., Biotechnology 4:1093-1096 (1986)).

Published Patent Applications EP 0292435, EP 0392225 and WO 93/07278 describe techniques for the preparation of callus and protoplasts of maize, transformation of protoplasts using PEG or electroporation, and the regeneration of maize plants from transformed protoplasts. Gordeon-Kamm et al., Plant Cell 2:603-618 (1990), and Fromm et al., Biotechnology 11:194-200 (1993), describe techniques for the transformation of elite inbred lines of maize by particle bombardment.

Transformation of rice can also be undertaken by direct gene transfer techniques utilizing protoplasts or particle bombardment. Protoplast-mediated transformation has been described for Japonica-types and Indica-types (Zhange et al., Plant Cell Rep. 7:739-384 (1988); Shimamoto et al. Nature 338:274-277 (1989); Datta et al. Biotechnology 8:736-740 (1990)). Both types are also routinely transformable using particle bombardment (Christou et al. Biotechnology 9:957-962 (1991)).

Patent Application EP 0 332 581 described techniques for the generation, transformation and regeneration of Pooideae protoplasts. Furthermore wheat transformation has been described by Vasil et al., Biotechnology 10:667-674 (1992), using particle bombardment into cells of type C long-term regenerable callus, and also by Vasil et al., Biotechnology 11:1553-1558 (1993), and Weeks et al., Plant Physiol. 102:1077-1084 (1993), using particle bombardment of immature embryos and immature embryo-derived callus.

Transformation of monocot cells such as Zea mays can be achieved by bringing the monocot cells into contact with a multiplicity of needle-like bodies on which these cells may be impaled, causing a rupture in the cell wall thereby allowing entry of transforming DNA into the cells. See U.S. Pat. No. 5,302,523. Transformation techniques applicable to both monocots and dicots are also disclosed in the following U.S. Pat. Nos. 5,240,855 (particle gun); 5,204,253 (cold gas shock accelerated microprojectiles); 5,179,022 (biolistic apparatus); 4,743,548 and 5,114,854 (microinjection); and 5,149,655, 5,120,657 (accelerated particle mediated transformation); 5,066,587 (gas driven microprojectile accelerator); 5,015,580 (particle-mediated transformation of soy bean plants); 5,013,660 (laser beam-mediated transformation); and 4,849,355 and 4,663,292.

In embodiments where the gene confers sensitivity to fungal disease or mycotoxin, transgenic plants may be prepared using standard techniques which result in downregulated expression or complete obliteration of gene expression. The downregulation may be the result of introduction of sequences which interfere with gene expression, such as antisense constructs, sense constructs, RNA silencing constructs, or RNA interference; or it may be the result of genomic disruptions of the sensitivity gene itself through the use of, for example, transposons, tilling, homologous recombination, or nonsense mutations.

Use of antisense nucleic acids is well known in the art. The antisense nucleic acid can be RNA, DNA, a PNA or any other appropriate molecule. An antisense nucleic acid can be produced, e.g., for a sensitivity gene by a number of well-established techniques (e.g., chemical synthesis of an antisense RNA or oligonucleotide (optionally including modified nucleotides and/or linkages that increase resistance to degradation or improve cellular uptake) or in vitro transcription). Antisense nucleic acids and their use are described, e.g., in U.S. Pat. Nos. 6,242,258; 6,500,615; 6,498,035; 6,395,544; and 5,563,050.

Another method to inhibit sensitivity gene expression is by sense suppression. Introduction of expression cassettes in which a nucleic acid is configured in the sense orientation with respect to the promoter has been shown to be an effective means by which to block the transcription of a desired target gene. See, e.g., Napoli et al. (1990), The Plant Cell 2:279-289, and U.S. Pat. Nos. 5,034,323, 5,231,020, and 5,283,184.

Catalytic RNA molecules or ribozymes can also be used to inhibit expression of sensitivity genes. It is possible to design ribozymes that specifically pair with virtually any target RNA and cleave the phosphodiester backbone at a specific location, thereby functionally inactivating the target RNA. In carrying out this cleavage, the ribozyme is not itself altered, and is thus capable of recycling and cleaving other molecules. The inclusion of ribozyme sequences within antisense RNAs confers RNA-cleaving activity upon them, thereby increasing the activity of the constructs.

Transgenic plants which include one or more inactivated sensitivity genes can also be produced by using RNA silencing or interference (RNAi), which can also be termed post-transcriptional gene silencing (PTGS) or cosuppression. In the context of this invention, “RNA silencing” (also called RNAi or RNA-mediated interference) refers to any mechanism through which the presence of a single-stranded or, typically, a double-stranded RNA in a cell results in inhibition of expression of a target gene comprising a sequence identical or nearly identical to that of the RNA, including, but not limited to, RNA interference, repression of translation of a target mRNA transcribed from the target gene without alteration of the mRNA's stability, and transcriptional silencing (e.g., histone acetylation and heterochromatin formation leading to inhibition of transcription of the target mRNA). In “RNA interference,” the presence of the single-stranded or double-stranded RNA in the cell leads to endonucleolytic cleavage and then degradation of the target mRNA. RNA interference is described e.g., in US patent application publications 2002/0173478, 2002/0162126, and 2002/0182223.

Any suitable transposable element may be used, including Class I (retrotransposons) or Class II (DNA transposons). Transposons useful in the present invention include members of the Mutator family of transposable elements, e.g., Robertson's Mutator (Mu) transposable elements; retrotransposons such as Ty1-copia, Ty3-gypsy, SINE-like, and LINE-like trasposons; and DNA transposable elements such as Ac, Tam1 and En/Spm.

Transposons may be delivered to cells, (e.g., through a sexual cross), transposition is selected for and the resulting insertion mutants are screened, e.g., for a phenotype of interest. Plants comprising disrupted sensitivity gene can be introduced into other plants by crossing the isolated or recombinant plants with a non-disrupted plant, e.g., by a sexual cross. Any of a number of standard breeding techniques can be used, depending upon the species to be crossed. The location of a transposon within a genome of an isolated or recombinant plant can be determined by known methods, e.g., sequencing of flanking regions as described herein. For example, a PCR reaction from the plant can be used to amplify the sequence, which can then be diagnostically sequenced to confirm its origin. Optionally, the insertion mutants are screened for a desired phenotype, such as the inhibition of expression or activity of sensitivity gene or fungal or mycotoxin resistance compared to a control plant.

Targeting Induced Local Lesions IN Genomics (TILLING) can also be used to generate transgenic plants with downregulated sensitivity gene expression. TILLING combines high density point mutations with rapid sensitive detection of the mutations. See, e.g., Oleykowski et al., (1998) Nucleic Acid Res. 26:4597-4602; and, Colbert et al., (2001) Plant Physiology 126:480-484.

Homologous recombination can also be used to inactivate one or more sensitivity genes by introducing mutations into the coding or non-coding sequences (including 5′ upstream, 3′ downstream, and intragenic regions). Homologous recombination has been demonstrated in plants. See, e.g., Puchta et al. (1994), Experientia 50: 277-284; Swoboda et al. (1994), EMBO J. 13: 484-489; Offringa et al. (1993), Proc. Natl. Acad. Sci. USA 90: 7346-7350; Kempin et al. (1997) Nature 389:802-803; and Terada et al., (2002) Nature Biotechnology, 20(10):1030-1034.

The transgenic plant cells or plant tissue are then grown into full plants in accordance with standard techniques. Transgenic seed can be obtained from transgenic flowering plants in accordance with standard techniques. Likewise, non-flowering plants such as potato and sugar beets can be propagated by a variety of known procedures. See e.g., Newell et al., Plant Cell Rep. 10:30-34 (1991) (disclosing potato transformation by stem culture).

The invention also includes transgenic plants and plant parts produced by any of the above techniques, non-naturally occurring seed obtained from such plants, as well as transformed protoplasts produced by the methods described above.

The present invention is described more fully by way of the following non-limiting examples. All references cited are hereby incorporated by reference in their entirety herein.

### EXAMPLES

### Example 1

### Identification of Sensitivity Genes

METHODS AND MATERIALS. The budding yeast, Saccharomyces cerevisiae, has been a powerful tool for genomic analysis of eukaryotic gene function, drug target identification and mechanism-of-action studies due to its facile genetics and highly conserved metabolic pathways. Around 30% of the known genes involved in human disease have yeast orthologs (7) Foury, F., Human genetic diseases: a cross-talk between man and yeast, Gene 1997, 195:1-10. Similarly, functional complementation of phenotypes in yeast allowed identification of the function of many plant genes (4, 16, 18, 21, 22) d'Enfert, et al., Cloning plant genes by complementation of yeast mutants, Methods Cell Biol 1995, 49:417-30; Mowla, et al., Yeast complementation reveals a role for an Arabidopsis thaliana late embryogenesis abundant (LEA)-like protein in oxidative stress tolerance, Plant J, 2006, 48:743-56; Rose, M. D., Isolation of genes by complementation in yeast, Methods Enzymol 1987, 152:481-504; Williamson, et al., Isolation of the structural gene for alcohol dehydrogenase by genetic complementation in yeast, Nature 1980, 283:214-6; Yang, et al., Functional characterization of allantoinase genes from Arabidopsis and a nonureide-type legume black locust, Plant Physiol 2004, 134:1039-49. The yeast deletion libraries have been used to identify the molecular basis for viral susceptibility, drug toxicity, and for bioactive compounds that affect numerous biological functions in eukaryotic cells (1, 10, 14, 19). Aouida, et al., A genome-wide screen in Saccharomyces cerevisiae reveals altered transport as a mechanism of resistance to the anticancer drug bleomycin, Cancer Res, 2004, 64:1102-9; Jiang, et al., Identification of essential host factors affecting tombusvirus RNA replication based on the yeast Tet promoters Hughes Collection. J Virol 2006, 80:7394-404; Luesch, et al., A genome-wide overexpression screen in yeast for small-molecule target identification, Chem Biol 2005, 12:55-63; Stepchenkova, et al., Genome-wide screening for genes whose deletions confer sensitivity to mutagenic purine base analogs in yeast, BMC Genet. 2005, 6:31.

The availability of several complete sets of deletion libraries in yeast provides a powerful approach to identify the genes that are critical for conferring resistance to trichothecenes. To identify the potential targets of these toxins, yeast (Saccharomyces cerevisiae) was used as a model organism to characterize the effect of mycotoxins on a broad range of genetic and biochemical processes in eukaryotes. A functional analysis of a large portion of the yeast genome is possible using a yeast knockout (YKO) library. The YKO library contains deletions of all non-essential genes in a homozygous diploid background (BY4743 parental strain) and is available from Open Biosystems. This library consists of 4720 unique strains representing 76% of the total known open reading frames (ORFs) in yeast. A screen using the YKO library was designed to identify genes that confer resistance to the mycotoxin trichothecin (T-cin) when deleted. Using this approach, novel targets of trichothecenes and pathways that control apoptosis upon exposure to mycotoxins cells can be identified. Understanding the mechanism of action of mycotoxins such as trichothecenes on eukaryotic cells would greatly benefit agriculture and human health, since these toxins are a major threat to the food supply. One potential benefit would be to devise new ways to enhance plant resistance to Fusarium and other fungal infections and thus reduce grain contamination.

Herein the applicant describes the genetic basis of eukaryotic cell susceptibility to mycotoxins such as the tricothecene family by identifying the genes, that when deleted, confer resistance to mycotoxins. The genes identified can be used to engineer resistance to diseases caused by mycotoxins such as Fusarium Head Blight and for developing effective approaches to prevent mycotoxin contamination of cereals. The present invention is useful in the areas of agriculture and human health since these toxins are a major threat to the food supply. The present invention can be advantageously applied to enhance plant resistance to certain fungal infections and thus reduce grain contamination.

The Saccharomyces cerevisiae genome homozygous gene deletion set was screened for resistance to 4 μM trichothecin. Sterile 96 deepwell plates (Costar 3960, 2 mL total volume) containing 450 μL YPD media with G418 sulfate (1.5 μg ml-1) were inoculated (10 μL) from the strain collection (stored in standard 96 well plates) obtained from Open Biosystems. The cultures were covered with a sterile, air permeable membrane (Breathe Easy strips from Research Products International Corp., catalog number 248738) and grown for 48 hours at 30° C. (nonshaking incubator). Glycerol stocks were made from this growth culture by adding 200 μL of a mixture of 50% glycerol/45% YP/5% dextrose. The growth culture media was mixed well by gently pipetting up and down and then distributed (200 each) to three 96 standard well plates (Nunc 267334). Plates were sealed with an aluminum seal (Nunc catalog number 276014), covered with a lid (Nunc 263339) and stored at −80° C. To generate the source plate (labeled Source Plate A) for the experiment, 10 μL of the prepared glycerol stock was used to inoculate a 96 deepwell plate containing 450 μL of YPD with G418 sulfate (1.5 μg ml−1). The plate (Source Plate A) was grown overnight to OD of 600 using a HiGro shaker (Gene Machines) at 350 RPM at 30° C. with oxygen supplied. The HiGro Shaker was a 24 deepwell plate capacity.

The experiment consists of replicate plating (3 replicates per library per treatment) into control 96 deep-well. The deep well library replication system from V&P Scientific, Inc. (VP408B) was used to inoculate plates containing 290 μL YPD liquid media supplemented with G418 sulfate (1.5 μg ml-1) from Source Plate A. The VP408B tool was used to thoroughly mix the culture prior to the transfer. Three control plates received 10 μL of 50% ethanol and three toxin treatment plates received 10 μL of Tcin (prepared in 50% ethanol). The final concentration of Tcin in the culture media is 4 μM. Plates were covered with the Breath Easy seal and grown for 20 hours in the HiGro shaker.

Growth rates were measured based on OD600 readings using the SpectraMax Plus spectrophotometer (96 well plate capacity). The OD600 for each treatment and control is obtained and a ratio of treated/untreated is calculated for each strain. A ratio of 1 indicates that the treatment had no effect on the strain (resistance). As a measure of variability, the coefficient of variation (standard deviation divided by the mean toxin treatment value) is reported. The current test is not designed to test for strains, which might be more susceptible to Tcin as the level of toxin chosen for this screening step was lethal to the parental strain (BY4743).

RESULTS. Our of 4720 homozygous diploid YKO strains which were screened, 122 strains were identified that showed resistance to 4 μM trichothecin (Tcin). 27 of these strains also showed resistance to 6 μM Tcin and 14 strains showed resistance to 8 Tcin. The experiments were performed in triplicate and descriptive statistics were generated for each strain. The criteria set to select the resistant strains from the bulk of the YKO data met the following requirements: 1)>0.25 ratio of toxin treatment/control, 2)<0.5 coefficient of variation (standard deviation of the toxin treatment divided by the toxin treatment mean).

At 4 μM, 122 (2.6%) YKO strains identified (54 cytosolic, 68 mitochondria related). At 6 μM, 27 (0.6%) YKO strains identified (18 cytosolic, 9 mitochondria related). At 8 μM (0.3%) YKO strains identified (11 cytosolic, 3 mitochondria related).

Applicants have identified genes involved in susceptibility to mycotoxins. These genes give resistance to trichothecene mycotoxins when their expression is inhibited. The genes represent novel candidate genes for resistance to diseases such as fusarium head blight (FHB) and reduced deoxynivalenol (DON) accumulation. Their expression can be inhibited using RNAi based or other approaches to obtain resistance to trichothecenes in cereals and other foodstuffs.

YKO strains which were found to be resistant at 8 μM tricothecin (Tcin), 27 YKO strains at 6 μM Tcin and 122 YKO strains at 4 μM Tcin. The genes represent novel candidate genes for resistance to fusarium head blight (FHB) and reduced deoxynivalenol (DON) accumulation. Resistance was conferred by deletion of genes involved in toxin uptake, intracellular transport, translation, RNA metabolism, defense response signaling and programmed cell death. The majority of identified genes were associated with mitochondria, implicating mitochondria in the toxin mechanism of action. These genes represent potential targets for engineering resistance to FHB and for developing effective approaches to prevent mycotoxin contamination of foods such as and not limited to starches, cereals and other grains.

Below is a summary of the 14 YKO strains which were found to be resistant to 8 μM Tcin. The genes represented may represent novel candidate genes for resistance to fusarium head blight (FHB) and reduced deoxynivalenol (DON) accumulation. Also see Table 1 for a summary of the 4 μM results, Table 2 for a summary of the 6 μM results, and Table 3 for 8 μM results.

Ribosome

RPL1B: N-terminally acetylated protein component of the large (60S) ribosomal subunit, nearly identical to Rpl1 Ap and has similarity to E. coli L1 and rat L10a ribosomal proteins; rpl1a rpl1b double null mutation is lethal.

Mitochondrial

TOF2: Nonessential mitochondrial protein of unknown function with sequence similarity to Net1p; identified as a topoisomerase I (Top1p) binding protein; displays synthetic genetic interactions with TOP1 (Topoisomerase I, nuclear enzyme that relieves torsional strain in DNA) and HPR1 (Subunit of THO/TREX complexes that couple transcription elongation with mitotic recombination and with mRNA metabolism and export, subunit of an RNA Pol II complex; regulates lifespan; involved in telomere maintenance).

AFG3: Component, with Yta12p, of the mitochondrial inner membrane m-AAA protease that mediates degradation of misfolded or unassembled proteins and is also required for correct assembly of mitochondrial enzyme complexes.

UPS1: Mitochondrial intermembrane space protein that regulates alternative processing and sorting of Mgm1p and other proteins; required for normal mitochondrial morphology; ortholog of human PRELI.

Endocytosis

YAP1802: (Yeast Assembly Polypeptide). Protein involved in clathrin cage assembly; binds Pan1p and clathrin; homologous to Yap1801p, member of the AP180 protein family. Likely role in endocytic pathway.

ER-Golgi Transport

FLC3: Putative FAD transporter, similar to Flc1p and Flc2p; localized to the ER.

Membrane-Related

MCH1: Protein with similarity to mammalian monocarboxylate permeases, which are involved in transport of monocarboxylic acids across the plasma membrane; mutant is not deficient in monocarboxylate transport.

LCB5: Minor sphingoid long-chain base kinase, paralog of Lcb4p responsible for few percent of the total activity, possibly involved in synthesis of long-chain base phosphates, which function as signaling molecules.

IZH4: Membrane protein involved in zinc metabolism, member of the four-protein IZH family, expression induced by fatty acids and altered zinc levels; deletion reduces sensitivity to excess zinc; possible role in sterol metabolism.

ICT1: Lysophosphatidic acid acyltransferase, responsible for enhanced phospholipid synthesis during organic solvent stress; null displays increased sensitivity to Calcofluor white; highly expressed during organic solvent stress, phosphatidic acid biosynthetic process.

Unknown Proteins

YDR157W: Dubious ORF; overlaps by 7 by with 3′ end of RPA14 (RNA Polymerase I subunit A 14)

CUE3: Protein of unknown function; has a CUE domain that binds ubiquitin, which may facilitate intramolecular monoubiquitination

YMR187c: Putative protein; physical interaction with MMS22 (protects against ionizing radiation). MMS22 (Methyl Methane Sulfonate sensitivity)-acts with Mms1p in a repair pathway that may be involved in resolving replication intermediates or preventing the damage caused by blocked replication forks

YNL011C: Uncharacterized ORF; genetic interactions with erg2 & erg6 (ergosterol biosynthesis)

TABLE 1
YKO hits at 4 μM: Classification and description

| | Gene | |
|---|---|---|
| ORF | Name | Description |
| YGL139W | FLC3 | Putative FAD transporter, similar to Flc1p and Flc2p; |
| | | localized to the ER |
| YDL054C | MCH1 | Protein with similarity to mammalian monocarboxylate |
| | | permeases, which are involved in transport of |
| | | monocarboxylic acids across the plasma membrane; mutant |
| | | is not deficient in monocarboxylate transport |
| YDR157W | | #N/A |
| YDL056W | MBP1 | Transcription factor involved in regulation of cell cycle |
| | | progression from G1 to S phase, forms a complex with |
| | | Swi6p that binds to MluI cell cycle box regulatory element |
| | | in promoters of DNA synthesis genes |
| YGL135W | RPL1B | N-terminally acetylated protein component of the large (60S) |
| | | ribosomal subunit, nearly identical to Rpl1Ap and has |
| | | similarity to E. coli L1 and rat L10a ribosomal proteins; |
| | | rpl1a rpl1b double null mutation is lethal |
| YKR010C | TOF2 | Nonessential mitochondrial protein of unknown function |
| | | with sequence similarity to Net1p; identified as a |
| | | topoisomerase I (Top1p) binding protein; displays synthetic |
| | | genetic interactions with TOP1 and HPR1 |
| YLR099C | ICT1 | Lysophosphatidic acid acyltransferase, responsible for |
| | | enhanced phospholipid synthesis during organic solvent |
| | | stress; null displays increased sensitivity to Calcofluor white; |
| | | highly expressed during organic solvent stress |
| YLR193C | UPS1 | Mitochondrial intermembrane space protein that regulates |
| | | alternative processing and sorting of Mgm1p and other |
| | | proteins; required for normal mitochondrial morphology; |
| | | ortholog of human PRELI |
| YGL110C | CUE3 | Protein of unknown function; has a CUE domain that binds |
| | | ubiquitin, which may facilitate intramolecular |
| | | monoubiquitination |
| YKR012C | | #N/A |
| YDL045W-A | MRP10 | Mitochondrial ribosomal protein of the small subunit |
| YLR262C-A | TMA7 | Protein of unknown that associates with ribosomes; null |
| | | mutant exhibits translation defects, altered polyribosome |
| | | profiles, and resistance to the translation inhibitor |
| | | anisomcyin |
| YDR533C | HSP31 | Possible chaperone and cysteine protease with similarity to |
| | | E. coli Hsp31; member of the DJ-1/ThiJ/PfpI superfamily, |
| | | which includes human DJ-1 involved in Parkinson's disease; |
| | | exists as a dimer and contains a putative metal-binding site |
| YPR047W | MSF1 | Mitochondrial phenylalanyl-tRNA synthetase alpha subunit, |
| | | active as a monomer, unlike the cytoplasmic subunit which |
| | | is active as a dimer complexed to a beta subunit dimer; |
| | | similar to the alpha subunit of E. coli phenylalanyl-tRNA |
| | | synthetase |
| YMR187C | | Putative protein; physical interaction with MMS22 (protects |
| | | against ionizing radiation) |
| YNL011C | | Uncharacterized ORF; genetic interactions with erg2 & erg6 |
| | | (ergosterol biosynthesis) |
| YKR046C | PET10 | Protein of unknown function that co-purifies with lipid |
| | | particles; expression pattern suggests a role in respiratory |
| | | growth; computational analysis of large-scale protein-protein |
| | | interaction data suggests a role in ATP/ADP exchange |
| YER017C | AFG3 | Component, with Yta12p, of the mitochondrial inner |
| | | membrane m-AAA protease that mediates degradation of |
| | | misfolded or unassembled proteins and is also required for |
| | | correct assembly of mitochondrial enzyme complexes |
| YLR260W | LCB5 | Minor sphingoid long-chain base kinase, paralog of Lcb4p |
| | | responsible for few percent of the total activity, possibly |
| | | involved in synthesis of long-chain base phosphates, which |
| | | function as signaling molecules |
| YGR241C | YAP1802 | Protein involved in clathrin cage assembly; binds Pan1p and |
| | | clathrin; homologous to Yap1801p, member of the AP180 |
| | | protein family |
| YDL044C | MTF2 | Mitochondrial matrix protein that interacts with an N- |
| | | terminal region of mitochondrial RNA polymerase (Rpo41p) |
| | | and couples RNA processing and translation to transcription |
| YMR310C | | #N/A |
| YOL101C | IZH4 | Membrane protein involved in zinc metabolism, member of |
| | | the four-protein IZH family, expression induced by fatty |
| | | acids and altered zinc levels; deletion reduces sensitivity to |
| | | excess zinc; possible role in sterol metabolism |
| YPR100W | MRPL51 | Mitochondrial ribosomal protein of the large subunit |
| YHR038W | RRF1 | Mitochondrial ribosome recycling factor, essential for |
| | | mitochondrial protein synthesis and for the maintenance of |
| | | the respiratory function of mitochondria |
| YDR519W | FPR2 | Membrane-bound peptidyl-prolyl cis-trans isomerase |
| | | (PPIase), binds to the drugs FK506 and rapamycin; |
| | | expression pattern suggests possible involvement in ER |
| | | protein trafficking |
| YPL219W | PCL8 | Cyclin, interacts with Pho85p cyclin-dependent kinase (Cdk) |
| | | to phosphorylate and regulate glycogen synthase, also |
| | | activates Pho85p for Glc8p phosphorylation |
| YJL119C | | #N/A |
| YOL095C | HMI1 | Mitochondrial inner membrane localized ATP-dependent |
| | | DNA helicase, required for the maintenance of the |
| | | mitochondrial genome; not required for mitochondrial |
| | | transcription; has homology to E. coli helicase uvrD |
| YGL064C | MRH4 | Mitochondrial ATP-dependent RNA helicase of the DEAD- |
| | | box family, plays an essential role in mitochondrial function |
| YFR019W | FAB1 | 1-phosphatidylinositol-3-phosphate 5-kinase; vacuolar |
| | | membrane kinase that generates phosphatidylinositol |
| | | (3,5)P2, which is involved in vacuolar sorting and |
| | | homeostasis |
| YDR529C | QCR7 | Subunit 7 of the ubiquinol cytochrome-c reductase complex, |
| | | which is a component of the mitochondrial inner membrane |
| | | electron transport chain; oriented facing the mitochondrial |
| | | matrix; N-terminus appears to play a role in complex |
| | | assembly |
| YDR065W | #N/A | #N/A |
| YGR283C | | #N/A |
| YJL152W | | #N/A |
| YGL119W | ABC1 | Protein required for ubiquinone (coenzyme Q) biosynthesis |
| | | and for respiratory growth; exhibits genetic interaction with |
| | | COQ9, suggesting a common function; similar to |
| | | prokaryotic proteins involved in early steps of ubiquinone |
| | | biosynthesis |
| YHR051W | COX6 | Subunit VI of cytochrome c oxidase, which is the terminal |
| | | member of the mitochondrial inner membrane electron |
| | | transport chain; expression is regulated by oxygen levels |
| YMR064W | AEP1 | Protein required for expression of the mitochondrial OLI1 |
| | | gene encoding subunit 9 of F1-F0 ATP synthase |
| YDL173W | | Putative protein of unknown function; green fluorescent |
| | | protein (GFP)-fusion protein localizes to the cytoplasm; |
| | | YDL173W is not an essential gene |
| YBR268W | MRPL37 | Mitochondrial ribosomal protein of the large subunit |
| YHR091C | MSR1 | Mitochondrial arginyl-tRNA synthetase |
| YDR296W | MHR1 | Protein involved in homologous recombination in |
| | | mitochondria and in transcription regulation in nucleus; |
| | | binds to activation domains of acidic activators; required for |
| | | recombination-dependent mtDNA partitioning |
| YDR519W | #N/A | #N/A |
| YKR006C | MRPL13 | Mitochondrial ribosomal protein of the large subunit, not |
| | | essential for mitochondrial translation |
| YBR122C | MRPL36 | Mitochondrial ribosomal protein of the large subunit; |
| | | overproduction suppresses mutations in the COX2 leader |
| | | peptide-encoding region |
| YBR179C | FZO1 | Mitochondrial integral membrane protein involved in |
| | | mitochondrial fusion and maintenance of the mitochondrial |
| | | genome; contains N-terminal GTPase domain |
| YGR101W | PCP1 | Mitochondrial serine protease required for the processing of |
| | | various mitochondrial proteins and maintenance of |
| | | mitochondrial DNA and morphology; belongs to the |
| | | rhomboid-GlpG superfamily of intramembrane peptidases |
| YBR126C | TPS1 | Synthase subunit of trehalose-6-phosphate |
| | | synthase/phosphatase complex, which synthesizes the |
| | | storage carbohydrate trehalose; also found in a monomeric |
| | | form; expression is induced by the stress response and |
| | | repressed by the Ras-cAMP pathway |
| YLR312W-A | MRPL15 | Mitochondrial ribosomal protein of the large subunit |
| YKL003C | MRP17 | Mitochondrial ribosomal protein of the small subunit; |
| | | MRP17 exhibits genetic interactions with PET122, encoding |
| | | a COX3-specific translational activator |
| YLR270W | DCS1 | Non-essential hydrolase involved in mRNA decapping, may |
| | | function in a feedback mechanism to regulate deadenylation, |
| | | contains pyrophosphatase activity and a HIT (histidine triad) |
| | | motif; interacts with neutral trehalase Nth1p |
| YLR199C | PBA1 | Protein involved in 20S proteasome assembly; forms a |
| | | heterodimer with Add66p that binds to proteasome |
| | | precursors; similar to human PAC1 constituent of the PAC1- |
| | | PAC2 complex involved in proteasome assembly |
| YFR032C-A | RPL29 | Protein component of the large (60S) ribosomal subunit, has |
| | | similarity to rat L29 ribosomal protein; not essential for |
| | | translation, but required for proper joining of the large and |
| | | small ribosomal subunits and for normal translation rate |
| YPL271W | ATP15 | Epsilon subunit of the F1 sector of mitochondrial F1F0 ATP |
| | | synthase, which is a large, evolutionarily conserved enzyme |
| | | complex required for ATP synthesis; phosphorylated |
| YPL104W | MSD1 | Mitochondrial aspartyl-tRNA synthetase, required for |
| | | acylation of aspartyl-tRNA; yeast and bacterial aspartyl-, |
| | | asparaginyl-, and lysyl-tRNA synthetases contain regions |
| | | with high sequence similarity, suggesting a common |
| | | ancestral gene |
| YDR509W | #N/A | #N/A |
| YNL284C | MRPL10 | Mitochondrial ribosomal protein of the large subunit; |
| | | appears as two protein spots (YmL10 and YmL18) on two- |
| | | dimensional SDS gels |
| YNL005C | MRP7 | Mitochondrial ribosomal protein of the large subunit |
| YDR377W | ATP17 | Subunit f of the F0 sector of mitochondrial F1F0 ATP |
| | | synthase, which is a large, evolutionarily conserved enzyme |
| | | complex required for ATP synthesis |
| YMR267W | PPA2 | Mitochondrial inorganic pyrophosphatase, required for |
| | | mitochondrial function and possibly involved in energy |
| | | generation from inorganic pyrophosphate |
| YPR116W | #N/A | #N/A |
| YCR003W | MRPL32 | Mitochondrial ribosomal protein of the large subunit |
| YCR071C | IMG2 | Mitochondrial ribosomal protein of the small subunit |
| YOL009C | MDM12 | Mitochondrial outer membrane protein, required for |
| | | transmission of mitochondria to daughter cells; component |
| | | of the Mdm10-Mdm12-Mmm1 complex involved in import |
| | | and assembly of outer membrane beta-barrel proteins |
| YER058W | PET117 | Protein required for assembly of cytochrome c oxidase |
| YGR219W | #N/A | #N/A |
| YCR046C | IMG1 | Mitochondrial ribosomal protein of the large subunit, |
| | | required for respiration and for maintenance of the |
| | | mitochondrial genome |
| YDR462W | #N/A | #N/A |
| YPL052W | OAZ1 | Regulator of ornithine decarboxylase (Spe1p), antizyme that |
| | | binds to Spe1p to regulate ubiquitin-independent |
| | | degradation; ribosomal frameshifting during synthesis of |
| | | Oaz1p and its ubiquitin-mediated degradation are both |
| | | polyamine-regulated |
| YJR101W | RSM26 | Mitochondrial ribosomal protein of the small subunit |
| YPR166C | MRP2 | Mitochondrial ribosomal protein of the small subunit |
| YER061C | CEM1 | Mitochondrial beta-keto-acyl synthase with possible role in |
| | | fatty acid synthesis; required for mitochondrial respiration |
| YGL218W | #N/A | #N/A |
| YBR251W | MRPS5 | Mitochondrial ribosomal protein of the small subunit |
| YBR282W | MRPL27 | Mitochondrial ribosomal protein of the large subunit |
| YJL102W | MEF2 | Mitochondrial elongation factor involved in translational |
| | | elongation |
| YGL064C | #N/A | #N/A |
| YNL073W | MSK1 | Mitochondrial lysine-tRNA synthetase, required for import |
| | | of both aminoacylated and deacylated forms of tRNA(Lys) |
| | | into mitochondria |
| YOR201C | MRM1 | Ribose methyltransferase that modifies a functionally |
| | | critical, conserved nucleotide in mitochondrial 21S rRNA |
| YPL118W | MRP51 | Mitochondrial ribosomal protein of the small subunit; |
| | | MRP51 exhibits genetic interactions with mutations in the |
| | | COX2 and COX3 mRNA 5′-untranslated leader sequences |
| YLL033W | IRC19 | Putative protein of unknown function; YLL033W is not an |
| | | essential gene but mutant is defective in spore formation; |
| | | null mutant displays increased levels of spontaneous Rad52 |
| | | foci |
| YMR287C | DSS1 | 3′-5′ exoribonuclease, component of the mitochondrial |
| | | degradosome along with the ATP-dependent RNA helicase |
| | | Suv3p; the degradosome associates with the ribosome and |
| | | mediates turnover of aberrant or unprocessed RNAs |
| YML110C | COQ5 | 2-hexaprenyl-6-methoxy-1,4-benzoquinone |
| | | methyltransferase, involved in ubiquinone (Coenzyme Q) |
| | | biosynthesis; localizes to the matrix face of the |
| | | mitochondrial inner membrane in a large complex with other |
| | | ubiquinone biosynthetic enzymes |
| YLR439W | MRPL4 | Mitochondrial ribosomal protein of the large subunit |
| YDL044C | #N/A | #N/A |
| YNL081C | SWS2 | Putative mitochondrial ribosomal protein of the small |
| | | subunit, has similarity to E. coli S13 ribosomal protein; |
| | | participates in controlling sporulation efficiency |
| YER050C | RSM18 | Mitochondrial ribosomal protein of the small subunit, has |
| | | similarity to E. coli S18 ribosomal protein |
| YKL134C | OCT1 | Mitochondrial intermediate peptidase, cleaves N-terminal |
| | | residues of a subset of proteins upon import, after their |
| | | cleavage by mitochondrial processing peptidase (Mas1p- |
| | | Mas2p); may contribute to mitochondrial iron homeostasis |
| YER087W | AIM10 | Protein with similarity to tRNA synthetases; non-tagged |
| | | protein is detected in purified mitochondria; null mutant |
| | | displays decreased frequency of mitochondrial genome loss |
| | | (petite formation), severe growth defect in minimal glycerol |
| | | media |
| YJL193W | | #N/A |
| YLL006W | MMM1 | Mitochondrial outer membrane protein, component of the |
| | | Mdm10-Mdm12-Mmm1 complex involved in import and |
| | | assembly of outer membrane beta-barrel proteins |
| YDL056W | #N/A | #N/A |
| YBL090W | MRP21 | Mitochondrial ribosomal protein of the large subunit; |
| | | MRP21 exhibits genetic interactions with mutations in the |
| | | COX2 and COX3 mRNA 5′-untranslated leader sequences |
| YOR241W | MET7 | Folylpolyglutamate synthetase, catalyzes extension of the |
| | | glutamate chains of the folate coenzymes, required for |
| | | methionine synthesis and for maintenance of mitochondrial |
| | | DNA |
| YLR295C | ATP14 | Subunit h of the F0 sector of mitochondrial F1F0 ATP |
| | | synthase, which is a large, evolutionarily conserved enzyme |
| | | complex required for ATP synthesis |
| YBR120C | CBP6 | Mitochondrial translational activator of the COB mRNA; |
| | | phosphorylated |
| YCR024C | SLM5 | Mitochondrial asparaginyl-tRNA synthetase |
| YMR293C | LRC6 | Putative protein similar to bacterial glutamyl-tRNA |
| | | amidotransferases; non-tagged protein is detected in purified |
| | | mitochondria; null mutant displays decreased frequency of |
| | | mitochondrial genome loss (petite formation) |
| YBR163W | #N/A | #N/A |
| YPL173W | MRPL40 | Mitochondrial ribosomal protein of the large subunit |
| YPL005W | AEP3 | Peripheral mitochondrial inner membrane protein, located on |
| | | the matrix face of the membrane; stabilizes the bicistronic |
| | | AAP1-ATP6 mRNA encoding subunits 6 and 8 of the ATP |
| | | synthase complex |
| YBR104W | YMC2 | Mitochondrial protein, putative inner membrane transporter |
| | | with a role in oleate metabolism and glutamate biosynthesis; |
| | | member of the mitochondrial carrier (MCF) family; has |
| | | similarity with Ymc1p |
| YER122C | GLO3 | ADP-ribosylation factor GTPase activating protein (ARF |
| | | GAP), involved in ER-Golgi transport; shares functional |
| | | similarity with Gcs1p |
| YGL246C | RAI1 | Nuclear protein that binds to and stabilizes the |
| | | exoribonuclease Rat1p, required for pre-rRNA processing |
| YLR139C | SLS1 | Mitochondrial membrane protein required for assembly of |
| | | respiratory-chain enzyme complexes III and IV; coordinates |
| | | expression of mitochondrially-encoded genes; may facilitate |
| | | delivery of mRNA to membrane-bound translation |
| | | machinery |
| YGR215W | RSM27 | Mitochondrial ribosomal protein of the small subunit |
| YJL132W | | #N/A |
| YHR147C | MRPL6 | Mitochondrial ribosomal protein of the large subunit |
| YAL010C | MDM10 | Subunit of both the Mdm10-Mdm12-Mmm1 complex and |
| | | the mitochondrial sorting and assembly machinery (SAM |
| | | complex); functions in both the general and Tom40p-specific |
| | | pathways for import and assembly of outer membrane beta- |
| | | barrel proteins |
| YPL057C | SUR1 | Probable catalytic subunit of a mannosylinositol |
| | | phosphorylceramide (MIPC) synthase, forms a complex with |
| | | probable regulatory subunit Csg2p; function in sphingolipid |
| | | biosynthesis is overlapping with that of Csh1p |
| YOL023W | IFM1 | Mitochondrial translation initiation factor 2 |
| YBR162W-A | #N/A | #N/A |
| YOR205C | LRC5 | Protein of unknown function; non-tagged protein is detected |
| | | in purified mitochondria in high-throughput studies; null |
| | | mutant displays decreased frequency of mitochondrial |
| | | genome loss and severe growth defect in minimal glycerol |
| | | media |
| YDL067C | #N/A | #N/A |
| YDL050C | #N/A | #N/A |
| YJL189W | RPL39 | Protein component of the large (60S) ribosomal subunit, has |
| | | similarity to rat L39 ribosomal protein; required for |
| | | ribosome biogenesis; exhibits genetic interactions with SIS1 |
| | | and PAB1 |
| YCR028C-A | RIM1 | Single-stranded DNA-binding protein essential for |
| | | mitochondrial genome maintenance; involved in |
| | | mitochondrial DNA replication |
| YGR220C | MRPL9 | Mitochondrial ribosomal protein of the large subunit |
| YBR101C | FES1 | Hsp70 (Ssa1p) nucleotide exchange factor, cytosolic |
| | | homolog of Sil1p, which is the nucleotide exchange factor |
| | | for BiP (Kar2p) in the endoplasmic reticulum |
| YJL208C | NUC1 | Major mitochondrial nuclease, has RNAse and DNA endo- |
| | | and exonucleolytic activities; has roles in mitochondrial |
| | | recombination, apoptosis and maintenance of polyploidy |
| YBR144C | #N/A | #N/A |
| YGL251C | HFM1 | Meiosis specific DNA helicase involved in the conversion of |
| | | double-stranded breaks to later recombination intermediates |
| | | and in crossover control; catalyzes the unwinding of |
| | | Holliday junctions; has ssDNA and dsDNA stimulated |
| | | ATPase activity |

TABLE 2
YKO hits at 6 μM: Classification and description

| | Gene | |
|---|---|---|
| ORF | Name | Description |
| YGL139W | FLC3 | Putative FAD transporter, similar to Flc1p and Flc2p; localized to |
| | | the ER |
| YDL054C | MCH1 | Protein with similarity to mammalian monocarboxylate |
| | | permeases, which are involved in transport of monocarboxylic |
| | | acids across the plasma membrane; mutant is not deficient in |
| | | monocarboxylate transport |
| YDR157W | | Dubious ORF; overlaps by 7 bp with 3′ end of RPA14 (RNA |
| | | Polymerase I subunit A14) |
| YDL056W | MBP1 | Transcription factor involved in regulation of cell cycle |
| | | progression from G1 to S phase, forms a complex with Swi6p that |
| | | binds to MluI cell cycle box regulatory element in promoters of |
| | | DNA synthesis genes |
| YGL135W | RPL1B | N-terminally acetylated protein component of the large (60S) |
| | | ribosomal subunit, nearly identical to Rpl1Ap and has similarity to |
| | | E. coli L1 and rat L10a ribosomal proteins; rpl1a rpl1b double null |
| | | mutation is lethal |
| YKR010C | TOF2 | Nonessential mitochondrial protein of unknown function with |
| | | sequence similarity to Net1p; identified as a topoisomerase I |
| | | (Top1p) binding protein; displays synthetic genetic interactions |
| | | with TOP1 and HPR1 |
| YLR099C | ICT1 | Lysophosphatidic acid acyltransferase, responsible for enhanced |
| | | phospholipid synthesis during organic solvent stress; null displays |
| | | increased sensitivity to Calcofluor white; highly expressed during |
| | | organic solvent stress |
| YLR193C | UPS1 | Mitochondrial intermembrane space protein that regulates |
| | | alternative processing and sorting of Mgm1p and other proteins; |
| | | required for normal mitochondrial morphology; ortholog of human |
| | | PRELI |
| YGL110C | CUE3 | Protein of unknown function; has a CUE domain that binds |
| | | ubiquitin, which may facilitate intramolecular monoubiquitination |
| YDL045W-A | MRP10 | Mitochondrial ribosomal protein of the small subunit |
| YDR533C | HSP31 | Possible chaperone and cysteine protease with similarity to E. coli |
| | | Hsp31; member of the DJ-1/ThiJ/PfpI superfamily, which includes |
| | | human DJ-1 involved in Parkinson's disease; exists as a dimer and |
| | | contains a putative metal-binding site |
| YPR047W | MSF1 | Mitochondrial phenylalanyl-tRNA synthetase alpha subunit, active |
| | | as a monomer, unlike the cytoplasmic subunit which is active as a |
| | | dimer complexed to a beta subunit dimer; similar to the alpha |
| | | subunit of E. coli phenylalanyl-tRNA synthetase |
| YMR187C | | Putative protein; physical interaction with MMS22 (protects |
| | | against ionizing radiation) |
| YNL011C | | Uncharacterized ORF; genetic interactions with erg2 & erg6 |
| | | (ergosterol biosynthesis) |
| YKR046C | PET10 | Protein of unknown function that co-purifies with lipid particles; |
| | | expression pattern suggests a role in respiratory growth; |
| | | computational analysis of large-scale protein-protein interaction |
| | | data suggests a role in ATP/ADP exchange |
| YGR241C | YAP1802 | Protein involved in clathrin cage assembly; binds Pan1p and |
| | | clathrin; homologous to Yap1801p, member of the AP180 protein |
| | | family |
| YLR260W | LCB5 | Minor sphingoid long-chain base kinase, paralog of Lcb4p |
| | | responsible for few percent of the total activity, possibly involved |
| | | in synthesis of long-chain base phosphates, which function as |
| | | signaling molecules |
| YER017C | AFG3 | Component, with Yta12p, of the mitochondrial inner membrane |
| | | m-AAA protease that mediates degradation of misfolded or |
| | | unassembled proteins and is also required for correct assembly of |
| | | mitochondrial enzyme complexes |
| YOL101C | IZH4 | Membrane protein involved in zinc metabolism, member of the |
| | | four-protein IZH family, expression induced by fatty acids and |
| | | altered zinc levels; deletion reduces sensitivity to excess zinc; |
| | | possible role in sterol metabolism |
| YDR519W | FPR2 | Membrane-bound peptidyl-prolyl cis-trans isomerase (PPIase), |
| | | binds to the drugs FK506 and rapamycin; expression pattern |
| | | suggests possible involvement in ER protein trafficking |
| YPL219W | PCL8 | Cyclin, interacts with Pho85p cyclin-dependent kinase (Cdk) to |
| | | phosphorylate and regulate glycogen synthase, also activates |
| | | Pho85p for Glc8p phosphorylation |
| YDR529C | QCR7 | Subunit 7 of the ubiquinol cytochrome-c reductase complex, |
| | | which is a component of the mitochondrial inner membrane |
| | | electron transport chain; oriented facing the mitochondrial matrix; |
| | | N-terminus appears to play a role in complex assembly |
| YLR270W | DCS1 | Non-essential hydrolase involved in mRNA decapping, may |
| | | function in a feedback mechanism to regulate deadenylation, |
| | | contains pyrophosphatase activity and a HIT (histidine triad) |
| | | motif; interacts with neutral trehalase Nth1p |
| YPL271W | ATP15 | Epsilon subunit of the F1 sector of mitochondrial F1F0 ATP |
| | | synthase, which is a large, evolutionarily conserved enzyme |
| | | complex required for ATP synthesis; phosphorylated |
| YPL052W | OAZ1 | Regulator of ornithine decarboxylase (Spe1p), antizyme that binds |
| | | to Spe1p to regulate ubiquitin-independent degradation; ribosomal |
| | | frameshifting during synthesis of Oaz1p and its ubiquitin- |
| | | mediated degradation are both polyamine-regulated |
| YPL057C | SUR1 | Probable catalytic subunit of a mannosylinositol |
| | | phosphorylceramide (MIPC) synthase, forms a complex with |
| | | probable regulatory subunit Csg2p; function in sphingolipid |
| | | biosynthesis is overlapping with that of Csh1p |
| YJL208C | NUC1 | Major mitochondrial nuclease, has RNAse and DNA endo- and |
| | | exonucleolytic activities; has roles in mitochondrial |
| | | recombination, apoptosis and maintenance of polyploidy |

TABLE 3
YKO hits at 8 μM: Classification and description

| | Gene | |
|---|---|---|
| ORF | Name | Description |
| YGL139W | FLC3 | Putative FAD transporter, similar to Flc1p and Flc2p; localized to |
| | | the ER |
| YDL054C | MCH1 | Protein with similarity to mammalian monocarboxylate permeases, |
| | | which are involved in transport of monocarboxylic acids across the |
| | | plasma membrane; mutant is not deficient in monocarboxylate |
| | | transport |
| YDR157W | | Dubious ORF; overlaps by 7 bp with 3′ end of RPA14 (RNA |
| | | Polymerase I subunit A14) |
| YGL135W | RPL1B | N-terminally acetylated protein component of the large (60S) |
| | | ribosomal subunit, nearly identical to Rpl1Ap and has similarity to |
| | | E. coli L1 and rat L10a ribosomal proteins; rpl1a rpl1b double null |
| | | mutation is lethal |
| YKR010C | TOF2 | Nonessential mitochondrial protein of unknown function with |
| | | sequence similarity to Net1p; identified as a topoisomerase I |
| | | (Top1p) binding protein; displays synthetic genetic interactions with |
| | | TOP1 and HPR1 |
| YLR099C | ICT1 | Lysophosphatidic acid acyltransferase, responsible for enhanced |
| | | phospholipid synthesis during organic solvent stress; null displays |
| | | increased sensitivity to Calcofluor white; highly expressed during |
| | | organic solvent stress |
| YGL110C | CUE3 | Protein of unknown function; has a CUE domain that binds |
| | | ubiquitin, which may facilitate intramolecular monoubiquitination |
| YMR187C | | Putative protein; physical interaction with MMS22 (protects against |
| | | ionizing radiation) |
| YNL011C | | Uncharacterized ORF; genetic interactions with erg2 & erg6 |
| | | (ergosterol biosynthesis) |
| YGR241C | YAP1802 | Protein involved in clathrin cage assembly; binds Pan1p and |
| | | clathrin; homologous to Yap1801p, member of the AP180 protein |
| | | family |
| YLR260W | LCB5 | Minor sphingoid long-chain base kinase, paralog of Lcb4p |
| | | responsible for few percent of the total activity, possibly involved in |
| | | synthesis of long-chain base phosphates, which function as |
| | | signaling molecules |
| YER017C | AFG3 | Component, with Yta12p, of the mitochondrial inner membrane m- |
| | | AAA protease that mediates degradation of misfolded or |
| | | unassembled proteins and is also required for correct assembly of |
| | | mitochondrial enzyme complexes |
| YOL101C | IZH4 | Membrane protein involved in zinc metabolism, member of the |
| | | four-protein IZH family, expression induced by fatty acids and |
| | | altered zinc levels; deletion reduces sensitivity to excess zinc; |
| | | possible role in sterol metabolism |
| YLR193C | UPS1 | Mitochondrial intermembrane space protein that regulates |
| | | alternative processing and sorting of Mgm1p and other proteins; |
| | | required for normal mitochondrial morphology; ortholog of human |
| | | PRELI |

### Example 2

### Further Characterization of Sensitivity Genes

Sensitivity to three different concentrations of T-cin was determined as outlined above. Results are shown in Table 4.

TABLE 4
YKO strains identified as resistant

| | Resistant | Resistant | Resistant |
|---|---|---|---|
| Gene Description | at 8 μM | at 12 μM | at 24 μM |
| Cell cycle | SPO13, PCL8 | SPO13, PCL8 | SPO13 |
| associated | | | |
| Cytosolic ribosome | TMA7, RPL1B | TMA7, RPL1B | TMA7 |
| associated | | | |
| Mitochondria | TOF2, MCH1, | TOF2, MCH1, | TOF2, |
| associated | UPS1, CBP6, | UPS1, CBP6, | UPS1 |
| | MRP51, PCP1, | MRP51, PCP1 | |
| | ATP17 | | |
| Membrane | ICT1, FLC3, | ICT1, FLC3, | ICT1 |
| associated/ | YAP1802, IZH4, | YAP1802, IZH4, | |
| Phospholipid | LCB5, VTS1 | LCB5, VTS1 | |
| synthesis | | | |
| Ubiquitination/ | CUE3, PBA1, | CUE3, PBA1 | |
| Proteasome Related | AFG3 | | |
| Unknown function | YNL011C, | YNL011C, | YNL011C |
| | YGR219W, PET10, | YGR219W, | |
| | YMR187C, | PET10, | |
| | YDL173W, | YMR187C, | |
| | YDR157W, IRC19 | YDL173W | |

Pulse-chase in vivo translation assays using [S35]-methionine was performed on the top 15 YKO T-cin resistant mutants along with BY4743. Translation inhibition was measured by in vivo [35S] methionine incorporation (pulse-chase experiment). Yeast cells were grown overnight to an A600 of approximately 1 OD in SD-Met containing 2% glucose. The synthetic media was prepared with glutamic acid (MSG) instead of ammonium sulfate as suggested by Tong and Boone (2006). Methods Mol Biol 313, 171-192. Previous research identified ammonium sulfate interfered with yeast sensitivity to antibiotics, such as G418 and that using a substitute such as monosodium glutamate (MSG) restored antibiotic selectivity (Webster and Dickson, R. C. (1983) Gene 26, 243-252; Cheng et al. (2000) Nucleic Acids Res 28, E108). Interestingly, the top 15 mutants grown in SD-met with ammonium sulfate as the nitrogen source became susceptible to 8 μM T-cin, a toxin concentration that all 15 are extremely resistant to when grown on complete media (YPD). Resistance was restored when grown in the SD-met media containing MSG. The equivalent of 10 OD Cells were collected by centrifugation and then resuspended in 6 mL SD-Met containing 2% glucose. However, when switched to SD-met with MSG as the nitrogen source, the expected resistance level of the mutants was restored. This 6 mL volume was divided equally into four 50 mL Falcon tubes (1.5 mL) each. To two of the tubes, 2 μL of 15 mM T-cin prepared in 50% ethanol was added (toxin treatment). This T-cin concentration was found from prior empirical work to inhibit the cytosolic translation rate of BY4743 by 80-90%. To the remaining two tubes, 2 μL of 50% ethanol was added (control treatment). The cultures were incubated in a 30° C. shaker for 1 h. The pulse began by the addition of 2 μL of [35S] methionine (PerkinElmer, NEG-009A, >1000 Ci/mmol) and the tubes were returned to the shaker/incubator for 10 min. The chase consisted of adding 38 μL 100 mM cold methionine for 10 min. Following the chase, an 800 μL sample was added to 200 μL of ice cold 100% trichloroacetic acid (TCA), placed on ice overnight and then heated at 80° C. for 20 minutes. Samples were quickly spun down and the labeled protein precipitate was collected onto pre-wetted (5% TCA) glass microfiber filters (Whatman Cat #1822024) using the Millipore 1225 sampling vacuum manifold. The filters were then washed 2× with 5% TCA, 2× with 95% ethanol, air dried and counted using a liquid scintillation counting assay. The cpm was normalized to the A600 reading and rates of translation were determined as cpm/A600.

In vivo [35S] methionine incorporation mitochondrial translation assay. Mitochondrial translation was measured using an assay developed by Fox et al., (1991) Methods Enzymol 194, 149-165. Modifications were made to accommodate the toxin exposure treatment. YPA-Raffinose (2%) yeast cultures (10 mL) were grown overnight at 30° C. The next morning, 5 mL of pre-warmed YPA-Raf was added to the culture. This was allowed to grow for 2-3 hours and the A600 OD was determined. Like the total protein assay described above, an equivalent of 10 OD cells were collected by centrifugation. The pellet was washed 2× in 5 mL SD-Met containing 2% raffinose using gulatmic acid (MSG) as the nitrogen source for the SD media. The cell pellet was then resuspended in 6 mL SD-Met Raf and divided equally into four 50 mL Falcon tubes. The toxin and control treatments were the same as for the total protein labeling experiment described above. The cultures were incubated in a 30° C. shaker for 1 h. Following this culture period, 50 μL of 7.5 mg/mL cycloheximide was added and the culture was incubated for 5 min at 30° C. The 10 min. pulse began by the addition of 2 μL of [35S] methionine followed by the addition of 38 μL cold methionine (100 mM) for a 10 min. chase, each phase incubated at 30° C. in the shaker. The samples were chilled in ice water for 5 min. and centrifuged at maximum speed for 1 min. At this time point, the chase period has been completed. The further process focuses on mitochondrial preparation. The cells are washed with 500 μL cold SHP buffer (0.6 M sorbitol, 20 mM HEPES-KOH pH7.4) with protease inhibitors and then resuspend in 250 μL SHP+ protease inhibitors. Yeast cells walls are disrupted by the addition of 1 vol. of cold glass beads and vortexed for an alternating period of 30 seconds and then 30 seconds on ice. This process is repeated 5 times and 150 μL of the supernatant is transferred to the new tube. Then, 250 μL of SHP buffer is added to the original tube and vortexed for an alternating period of 30 seconds and 30 seconds on ice. This process w is repeated 2 times and the supernatant is again added to the new tube. Finally, all the supernatant fractions are combined and spun at 4° C. at 3000 rpm for 5 min to pellet cell debris. An aliquot of 100 μL of supernatant is saved to protein analysis by the Bradford assay. The remaining supernatant is transferred to a new tube and spun at 4° C. at a maximum speed for 10 minutes. The pellet represents the mitochondrial fraction. The pellet is resuspended in 150 μL of 2× protein solubilizing buffer (Laemmli buffer) and heated for 65° C. for 5 minutes. A 5 μL aliquot of solution is added to 5 mL of scintillation fluid and counted with the scintillation counter. This count gives an indication of the amount of [S35] incorporated into the mitochondrial protein. The protein solution is loaded onto 15% gels and run at 50 mA for 1-1.5 hours. The gel is then stained with Coomassie blue, photographed, incubated with EN3HANCE (PerkinElmer) for 1 hr, washed with a water/glycerol/PEG solution for 15-30 minutes to precipitate the scintillators, and then dried under vacuum for approximately 1 hour. The dried gel is then exposed to film for 3-7 days.

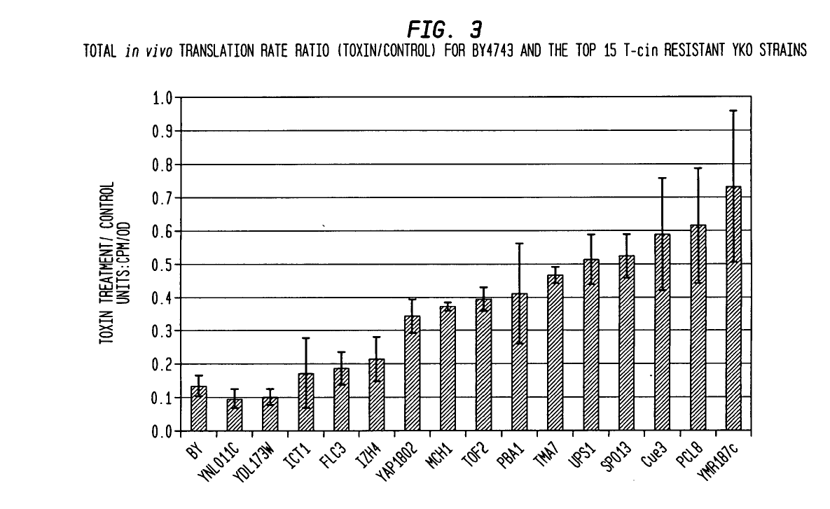

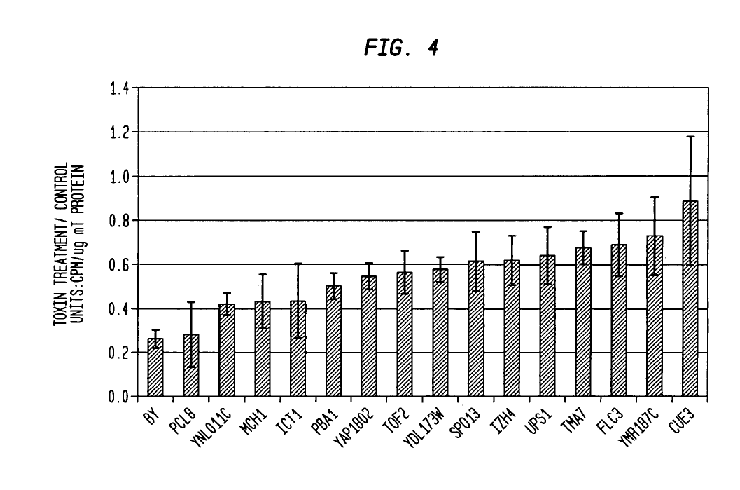

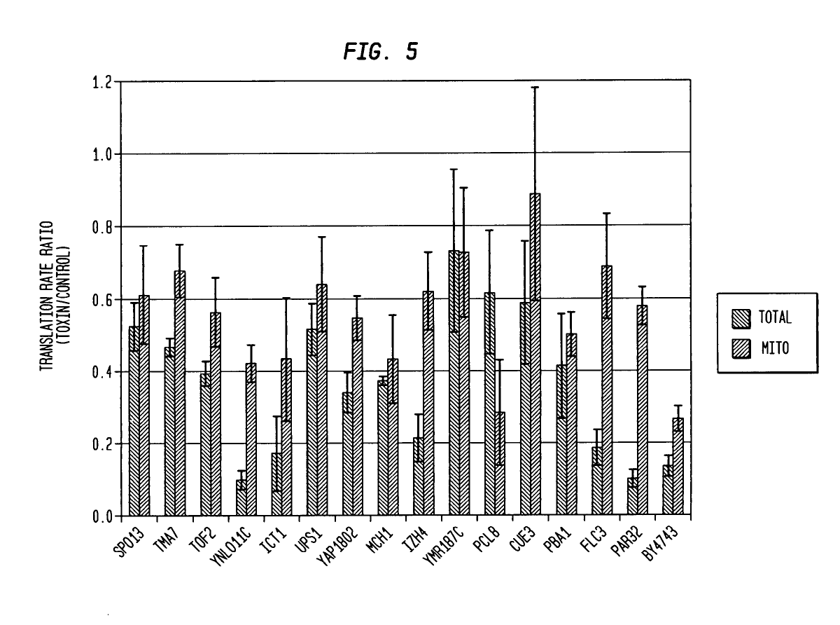

Pulse-chase in vivo translation assays using [S35]-methionine was performed on the top 15 YKO T-cin resistant mutants along with BY4743. Total translation (FIG. 3) and mitochondrial translation (FIG. 4) were measured for all 16 strains. A translation ratio was calculated for each strain based on the translation rate of the strain exposed to 8 μM T-cin/OD cells divided by the translation rate of that same strain grown without the toxin. In general, the interstrain-relative translation rates for the top 15 YKO strains were less inhibited by T-cin compared to the isogenic BY4743. A few top resistant YKO strains show translation rates as low as the most susceptible strain (BY4743) for total translation or the mitochondrial translation. However, no top resistant YKO deletion strain shows inhibition for both total translation and mitochondrial translation (FIG. 5 and Table 5).

TABLE 5
Summary of the in vivo translation pulse-
chase experiment (total and mitochondrial)

| YKO strains that display a total | YKO strains that display a |
|---|---|
| translation rate similar to or below | mitochondrial translation rate |
| BY4743 upon exposure to T-cin (8 | similar to BY4743 upon exposure |
| μM/OD cells) | to T-cin (8 μM/OD cells) |
| YNL011C | PCL8 |
| ICT1 | |
| IZH4 | |
| FLC3 | |
| PAR32 | |

The finding that growth in media containing non-fermentable sugar increases susceptibility, the identification of a large fraction of mitochondrial-related YKOs, and that fact that T-cin is able to inhibit mitochondrial translation (show that T-cin is capable of interfering with the activity of mitochondria in addition to inhibiting cytoplasmic protein synthesis. However, while many of the identified resistant YKOs at 4 μM T-cin, none of the top T-cin resistant YKO strains are rho⁰ or rho⁻, which indicates a potential nuclear-mitochondrial interaction or energeics for the top mutants.

### Example 3

### Identification of Resistance Genes

Materials and Methods. A high-throughput screening approach to screen the YKO library was developed based on a modified version of a protocol by Binder Binder, J., Nat Toxins 1999.7:401-6. The screen uses an in vivo liquid culture system laid out in a 96 deep-well plate format. This is the same format as the 96 well plates provided by Open Biosystems for the YKO glycerol stocks. The availability of several complete sets of deletion libraries in yeast provides a powerful approach to identify the genes that are critical for conferring resistance to trichothecenes. The parental strain of the homozygous diploid knockout library, BY4743, is particularly sensitive to the trichothecene trichothecin (T-cin). An IC₅₀ assay identified 3 μM T-cin to be sub lethal below which the toxin had no effect on the growth of BY4743. Thus a non lethal concentration of 2 μM was selected for the subsequent screening experiments. The high throughput, comprehensive screen of the yeast genome aims to identify and characterize genes, which play a role in trichothecene mycotoxin uptake, intracellular trafficking, signaling, metabolism, DNA/RNA/ribosome interactions, and ultimately the apoptosis response.

The Saccharomyces cerevisiae genome homozygous gene deletion set was screened for sensitivity to 2 μM trichothecin. Sterile 96 deepwell plates (Costar 3960, 2 mL total volume) containing 450 μL YPD media with G418 sulfate (1.5 pg ml⁻¹) were inoculated (10 μL) with the strain collection (stored in standard 96 well plates) obtained from Open Biosystems. The cultures were covered with a sterile, air permeable membrane (Breathe Easy strips from Research Products International Corp., catalog number 248738) and grown for 48 hours at 30° C. (non-shaking incubator). Glycerol stocks were made from this growth culture by adding 200 μL of a mixture of 50% glycerol/45% YP/5% dextrose. The growth culture media was mixed well by gently pipetting up and down and then distributed (200 μL each) to three 96 standard well plates (Nunc 267334). Plates were sealed with an aluminum seal (Nunc catalog number 276014), covered with a lid (Nunc 263339) and stored at −80° C. To generate the source plate (labeled Source Plate A) for the experiment, 10 pL of the prepared glycerol stock was used to inoculate a 96 deepwell plate containing 450 μL of YPD with G418 sulfate (1.5 μg ml⁻¹). The plate (Source Plate A) was grown overnight using a HiGro shaker (Gene Machines) at 350 RPM at 30° C. with oxygen supplied. The HiGro Shaker has a 24 deepwell plate capacity. To prepare YPD solid plates, 50 mL YPD agar was poured into each Omni Tray (VP 413, Nalge Nunc). Prior to pouring, 6.654 of 50% ethanol was added to YPD agar for the control plates and 6.654, of Tcin (prepared in 50% ethanol) for the toxin treatment plates. The final concentration of Tcin in each solid plate is 2 μM.

The experiment consists of replicate printing (3 replicates per library per treatment) cultures from Source Plate A onto solid plates. Cultures were printed twice onto each solid plate and grown for 36 hours in a 30° C. incubator. For printing, a 96 solid pin multi-blot replicator from V&P Scientific, Inc. (VP408B) is used to transfer the mutant strains from Source Plate A onto each solid plate with or without the 2 μM Tcin. The VP408B replicator was used to thoroughly mix the culture prior to the transfer.

After 20 hours the mutants form colonies on the solid plates at each spot where they were printed by the VP408replicator. However, some mutants formed colonies in the control plates and showed either no growth or little growth in the toxin treatment plates. These mutants were scored as more sensitive to the toxin than the wild type due to their inability to grow at this non lethal concentration of 2 μM, which is otherwise tolerated by the parental strain.

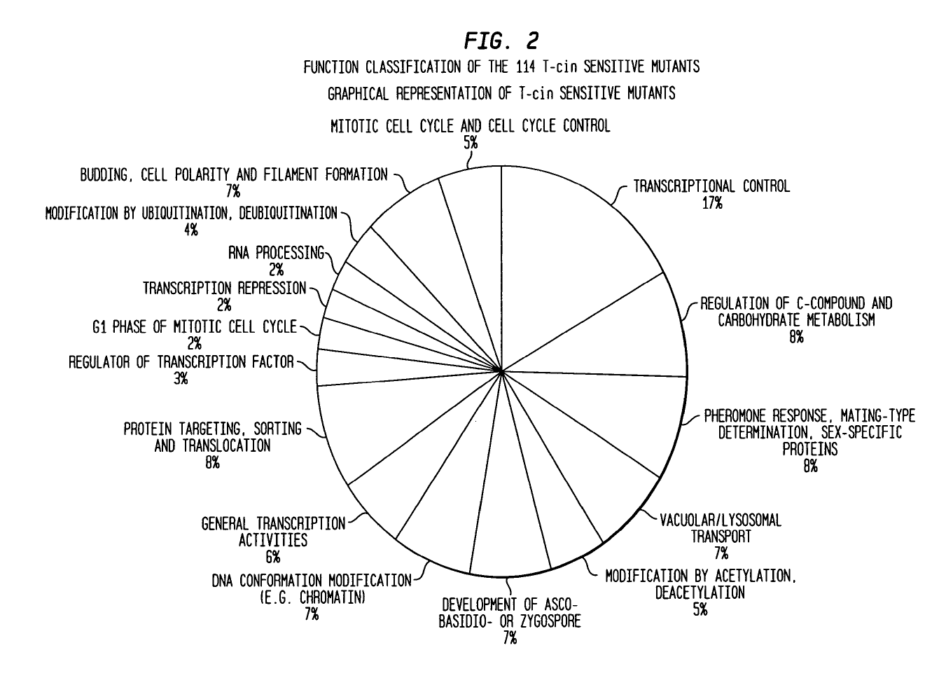

114 YKO strains (over 2% of the entire YKO homozygous diploid collection) were identified ranging from sensitive to hypersensitive at 2 μM T-cin concentration (Table 4) and showed little to now growth at this normally non-lethal concentration. The selection is based on two independent screenings with each screening done in duplicates. Functional classification of the select mutants revealed major cluster of genes associated with protein synthesis, vacuolar protein sorting & degradation, chemical modification, and chromatin remodeling (FIG. 2). The absence of these genes has rendered these mutants sensitive to T-cin and thus expression and the overexpression of these missing genes may induce resistance to T-cin at higher concentrations.

TABLE 6

| Systematic | Common |
|---|---|
| Name | Name |
| YAL002W | VPS8 |
| YAL013W | DEP1 |
| YAL021C | CCR4 |
| YAL047C | SPC72 |
| YBL058W | SHP1 |
| YBL071W-A | KTI11 |
| YBL093C | ROX3 |
| YBR023C | CHS3 |
| YBR081C | SPT7 |
| YBR112C | CYC8 |
| YBR267W | REI1 |
| YBR279W | PAF1 |
| YBR289W | SNF5 |
| YCR020W-B | HTL1 |
| YCR044C | PER1 |
| YCR094W | CDC50 |
| YDL013W | SLX5 |
| YDL023C | |
| YDL075W | RPL31A |
| YDL106C | PHO2 |
| YDR049W | |
| YDR264C | AKR1 |
| YDR266C | |
| YDR433W | |
| YDR442W | |
| YDR495C | VPS3 |
| YDR532C | |
| YEL044W | IES6 |
| YER070W | RNR1 |
| YER087W | AIM10 |
| YER151C | UBP3 |
| YFR010W | UBP6 |
| YGL012W | ERG4 |
| YGL038C | OCH1 |
| YPL050C | MNN9 |
| YPL084W | BRO1 |
| YPL129W | TAP14 |
| YPL254W | HFI1 |
| YPL268W | PLC1 |
| YGL058W | RAD6 |
| YGL070C | RPB9 |
| YGR006W | PRP18 |
| YGR063C | SPT4 |
| YGR092W | DBF2 |
| YGR104C | SRB5 |
| YGR196C | FYV8 |
| YGR262C | BUD32 |
| YGR271W | SLH1 |
| YHL025W | SNF6 |
| YHR013C | ARD1 |
| YHR030C | SLT2 |
| YJL028W | |
| YJL095W | BCK1 |
| YJL129C | TRK1 |
| YJL175W | |
| YJL184W | GON7 |
| YJR090C | GRR1 |
| YJR102C | VPS25 |
| YKL002W | DID4 |
| YKL139W | CTK1 |
| YKL213C | DOA1 |
| YKR023W | |
| YKR082W | NUP133 |
| YKR093W | PTR2 |
| YLR025W | SNF7 |
| YLR027C | AAT2 |
| YLR226W | BUR2 |
| YLR234W | TOP3 |
| YLR240W | VPS34 |
| YLR322W | VPS65 |
| YLR330W | CHS5 |
| YLR337C | VRP1 |
| YLR338W | OPI9 |
| YPR072W | NOT5 |
| YPR173C | VPS4 |
| YPL042C | SSN3 |
| YPL045W | VPS16 |
| YKR004C-A | |
| YLR370C | ARC18 |
| YLR417W | VPS36 |
| YML008C | ERG6 |
| YML062C | MFT1 |
| YML094W | GIM5 |
| YML112W | CTK3 |
| YMR032W | HOF1 |
| YMR077C | VPS20 |
| YMR202W | ERG2 |
| YMR263W | SAP30 |
| YNL054W | VAC7 |
| YNL064C | YDJ1 |
| YNL084C | END3 |
| YNL097C | PHO23 |
| YNL133C | FYV6 |
| YNL140C | |
| YNL147W | LSM7 |
| YNL229C | URE2 |
| YNL236W | SIN4 |
| YNR051C | BRE5 |
| YNR052C | POP2 |
| YOL004W | SIN3 |
| YOL051W | GAL11 |
| YOL068C | HST1 |
| YOL086C | ADH1 |
| YOL148C | SPT20 |
| YOR026W | BUB3 |
| YOR035C | SHE4 |
| YOR036W | PEP12 |
| YOR124C | UBP2 |
| YOR152C | |
| YOR290C | SNF2 |
| YOR298C-A | MBF1 |
| YOR302W | |
| YML013C-A | |

TABLE 7
Sensitivity genes

| Systematic | Common | |
|---|---|---|
| Name | Name | Sequence |
| YLL033W | IRC19 | ATGCGTAAGCCTTCTATTACGATAACAACAGCAAAAGCTATTATAACTCCAGACTATACT |
| | | CTTATTAAATCGCATTCTAAATACCAACTTCCTTCACGTTTTCAAAAGCTAGATGCAGAC |
| | | TCACCTGAGAGAAGTACAGTTGTTAAGCTATTTTACCGCCGATTTATGAGGCTTAAGCCT |
| | | TTCATATCGAATGTGAAAATGGTTAAAGATACTTACAGGGATTATGTTCGATATAAGTTT |
| | | ATGAAAGAAAACTACGAACTCAAACGATACTTGGTATTCAATCCTGATGGCTTGAGGTCG |
| | | AAAATCAATCTTGAGCTTCTCAGCAACACGAAATGTTGTGAAAGGATACTCCCCGTGACT |
| | | GAGATGCAAAGAACCTTGGAATTTGTTCTGAAGAGCTGTTCATACTTGCCCGAAACAAAA |
| | | GTTCAAAAGTGGGATATTGCAAGGGACAACACATATTGCAGGCAAATTTTAAAGAACCTT |
| | | TTAACAATGCAATATGAAAAGTACAGATCGATTTTGCATAGAGGAATAGGCCATGATGAG |
| | | CTTGACGTCAAATTCTCTCATTTGAAAACTACTTCTAGTCCGCTAACTAAGCTAAATAAA |
| | | ACCGAAAAGAAAAAGATTCCGCTTTTCAAAGTATTTAGTGATTTCGATACAACATTGATA |
| | | TATCTCAATGAGACATTAGGTACAAGGTTATAA |
| YDR377W | ATP17 | ATGATTTTTAAACGTGCAGTATCTACATTGATTCCTCCAAAAGTTGTGTCTTCCAAGAAT |
| | | ATAGGTTCGGCACCAAATGCCAAGCGCATTGCTAATGTTGTTCACTTTTATAAGTCTTTG |
| | | CCTCAAGGACCAGCACCAGCCATCAAGGCTAACACTAGATTGGCCAGATACAAAGCCAAG |
| | | TACTTTGATGGGGATAATGCTAGTGGTAAACCATTGTGGCATTTTGCTCTAGGTATAATT |
| | | GCCTTTGGCTATTCCATGGAATATTATTTTCATTTGAGACATCATAAAGGTGCGGAAGAG |
| | | CATTGA |
| YDR157W | — | ATGAGTGAGACCTGTAGCAGCAGCCTTGCCCTTTTACACAAAATCCTACATATCCATTCA |
| | | CATACGCCTTCTGTATACTACAATATATGCATATCAGTTAGAATTCTGACATCTGAACGT |
| | | CTTCAGTGCTTTTTTTTCTCTTTCTTTCCAGATCCAAATATCACCGGCAGCGGCCTCAAA |
| | | GTACCCGGTTTTCTTTTTTTTCACACTTTTTTTTTTTCCAAATCATGTTGTCAAGCATTG |
| | | ATTGACTCATTTTCCTCTGACTACTACCAGTTCAAAATGTTAGAGAAAAATAGAAAAGCA |
| | | GAAAAAATAAATAAAAGAACCATATTTATTTGTTCATTCACTTTCGAGTATAAAATTAAA |
| | | TCTTGTTTTTCTTGTTTTCACTTAAGTACACATACAAATTAA |
| YER017C* | AFG3; | ATGATGATGTGGCAACGATATGCAAGGGGTGCGCCACGCTCATTGACATCACTCTCATTT |
| | YTA10 | GGTAAAGCTAGCCGCATATCAACAGTGAAGCCAGTGCTTCGCTCGCGCATGCCAGTTCAC |
| | | CAGCGTTTGCAGACGTTATCTGGTCTGGCAACCAGAAACACAATACACCGTTCTACCCAA |
| | | ATACGTTCCTTCCATATCTCATGGACAAGGTTAAATGAAAATAGGCCAAACAAAGAGGGT |
| | | GAGGGCAAGAACAATGGTAATAAAGATAATAATAGCAATAAAGAAGATGGCAAAGACAAA |
| | | AGAAATGAGTTTGGTTCATTATCAGAATACTTCAGATCTAAGGAATTTGCTAATACGATG |
| | | TTTTTGACCATCGGATTCACAATTATATTTACTTTGCTCACACCTTCCAGTAACAACTCA |
| | | GGAGACGACTCTAACCGCGTCTTGACTTTCCAGGATTTCAAAACAAAATACCTGGAGAAG |
| | | GGTCTTGTGTCCAAGATTTACGTCGTTAATAAGTTTCTCGTAGAGGCCGAATTAGTCAAT |
| | | ACAAAGCAAGTTGTATCGTTCACCATTGGTTCAGTAGATATTTTTGAGGAACAGATGGAC |
| | | CAGATCCAGGACCTCTTGAACATTCCTCCTCGGGATCGTATCCCCATCAAATACATTGAG |
| | | AGATCTTCTCCCTTCACATTTTTGTTCCCCTTCCTGCCCACCATCATTCTGCTTGGTGGC |
| | | CTTTACTTCATAACCAGAAAAATAAATAGTTCACCACCAAATGCCAATGGTGGTGGTGGA |
| | | GGAGGCCTCGGCGGCATGTTTAATGTTGGAAAATCCAGAGCAAAGCTCTTCAATAAGGAA |
| | | ACAGACATTAAAATTTCATTTAAAAATGTTGCCGGTTGTGATGAAGCTAAACAGGAAATC |
| | | ATGGAATTTGTTCACTTTTTGAAGAACCCAGGTAAGTACACTAAATTGGGTGCCAAGATT |
| | | CCACGAGGCGCTATTCTTTCTGGACCCCCAGGTACCGGTAAGACTCTCTTGGCCAAGGCC |
| | | ACCGCAGGTGAGGCGAACGTGCCCTTCTTGTCAGTATCAGGTTCTGAGTTCGTCGAAATG |
| | | TTCGTTGGGGTGGGTGCTTCACGTGTAAGAGACTTGTTTACTCAAGCAAGGTCTATGGCC |
| | | CCCTCGATTATCTTTATAGATGAAATTGACGCTATCGGTAAAGAAAGAGGCAAAGGCGGC |
| | | GCTCTCGGTGGCGCTAACGATGAAAGAGAAGCTACGCTGAATCAATTATTGGTGGAAATG |
| | | GACGGATTCACTACTTCCGACCAAGTCGTAGTCCTTGCGGGTACAAATAGGCCCGATGTG |
| | | TTGGATAATGCTTTGATGAGACCGGGAAGGTTCGATAGACATATCCAAATTGATTCTCCT |
| | | GATGTCAATGGTAGGCAGCAAATATACCTGGTTCACTTGAAAAGACTGAATCTGGATCCC |
| | | CTTCTTACAGATGATATGAATAATCTTTCTGGGAAATTGGCTACGCTTACTCCAGGTTTT |
| | | ACTGGTGCAGATATCGCTAATGCTTGTAATGAGGCTGCATTAATCGCTGCCAGACATAAT |
| | | GACCCATATATCACTATCCATCACTTTGAGCAAGCCATTGAAAGAGTCATTGCGGGACTA |
| | | GAGAAGAAAACAAGAGTCCTTTCTAAGGAAGAAAAAAGGTCAGTGGCCTATCATGAGGCA |
| | | GGACATGCCGTTTGTGGTTGGTTTCTAAAATATGCGGATCCACTTCTGAAAGTAAGCATC |
| | | ATCCCGCGTGGACAAGGTGCTTTAGGCTATGCCCAGTACCTACCACCGGATCAATATTTG |
| | | ATATCTGAGGAGCAATTCAGACATAGAATGATCATGGCTCTTGGTGGCCGTGTTTCTGAG |
| | | GAGCTACATTTTCCATCGGTGACTAGCGGTGCTCATGATGATTTCAAAAAGGTTACACAG |
| | | ATGGCAAATGCCATGGTTACATCCCTAGGAATGTCACCCAAGATTGGCTACCTGTCGTTT |
| | | GATCAGAATGATGGAAACTTCAAAGTTAACAAACCCTTCAGTAATAAAACGGCAAGAACC |
| | | ATTGATTTAGAAGTTAAATCTATAGTAGATGATGCACACCGAGCTTGTACAGAATTGCTA |
| | | ACCAAAAATTTGGACAAAGTCGATCTTGTCGCAAAAGAATTGCTACGTAAGGAAGCAATT |
| | | ACTAGAGAAGACATGATTAGATTATTGGGTCCAAGGCCATTCAAGGAAAGGAACGAGGCT |
| | | TTTGAAAAATATTTGGATCCAAAGAGCAATACCGAACCGCCTGAGGCACCTGCAGCAACA |
| | | AATTAA |
| YGL135W | RPL1B; | ATGTCTAAGATCACCTCTTCTCAAGTCAGAGAACACGTCAAGGAATTGTTGAAGTATTCC |
| | SSM2 | AACGAAACCAAGAAGAGAAACTTCTTGGAAACCGTCGAACTACAAGTCGGTTTGAAGAAC |
| | | TATGACCCTCAAAGAGACAAGCGTTTCTCTGGTTCTTTGAAGTTGCCAAACTGTCCAAGA |
| | | CCAAACATGTCCATCTGTATCTTCGGTGATGCTTTCGATGTTGACAGAGCTAAGTCTTGC |
| | | GGTGTTGACGCTATGTCCGTCGATGACTTGAAGAAGTTGAACAAGAACAAGAAGTTAATC |
| | | AAGAAGTTGTCTAAGAAGTACAACGCTTTCATTGCTTCCGAAGTTTTGATCAAGCAAGTT |
| | | CCAAGACTATTGGGTCCTCAATTGTCCAAGGCTGGTAAGTTCCCAACCCCAGTTTCTCAC |
| | | AACGATGACTTGTACGGTAAGGTCACTGATGTCAGATCTACCATCAAGTTCCAATTGAAG |
| | | AAGGTCTTGTGTTTGGCTGTTGCCGTTGGTAACGTTGAAATGGAAGAAGACGTTTTGGTT |
| | | AACCAAATCTTGATGTCTGTTAACTTCTTTGTTTCTTTGTTGAAGAAGAACTGGCAAAAT |
| | | GTTGGTTCCTTGGTTGTTAAGTCCTCCATGGGTCCAGCTTTCAGATTGTACTAA |
| YGR219W | — | ATGTACATGATTGTAGTAAAATACTTATACGCCTTATGCTCAAGTTTTTTTGATTGCATC |
| | | TTGAATTTTAACGAAACTGTTTTTGGGCCCAGCCACAGAGCCTTTAACCCAAATAACATT |
| | | ATTTTCATCGTCGACTTTCAAAACTTTAACATTTTGGATAGTAACATGCTCGTTTCCCAT |
| | | ATGACCAGGCATCTTTCTTCCTGGCAGTACCCTACCTGGGTCTTGGTTTTGACCATAAGA |
| | | ACCACCGTGTCTATGCATAATAGATGTACCATGAGATGCTCTCAAACCCTTGAATCCATA |
| | | CCTTTTCATCACACCAGTAAAACCTTTGCCTTTGCTGACTGA |
| YLR260W | LCB5 | ATGACTTTGAAACCTTCAAAGAGACGTAAGGGCAGGTCTCGCCATTCCAGGAAGAAGCAA |
| | | ATAACGTCAGCGATACTGACTGAGGAAGGAATAATGATCAAGGCAAAACCATCAAGTCCT |
| | | TACACATACGCAAATAGAATGGCAGATAAACGAAGTCGCAGCAGCATTGACAACATCAGT |
| | | AGAACTAGCTTTCAAAGCAACATCAGTAGAACTAGCTTTCAAAGCAACAGTGATAACAAC |
| | | AGTATATTTGAAACGGCTTCACTAATTAGCTGTGTTACCTGTTTAAGCGATACTGATACA |
| | | ATAGACAGATCGGAAACATCGACAACGGATACAAGTAAAGATGATCTTTCTGCTAATCCA |
| | | AAACTTCATTATCCTTCGGTGAATGGTCAATTGCCAGCAAACACCGTTATCCCCTATGGA |
| | | CGAATTCTGGATGCCAGATACATTGAAAAGGAACCTCTGCATTATTATGATGCCAATTCA |
| | | TCACCCAGTTCACCTTTAAGCAGCTCAATGAGTAACATTAGTGAAAAGTGTGATCTTGAT |
| | | GAATTAGAGTCTTCCCAAAAAAAAGAAAGGAAGGGCAACTCGCTATCGCGAGGAAGTAAC |
| | | AGTAGTAGTAGCCTCCTGACTTCCAGATCTCCTTTTACGAAACTAGTAGAGGTTATATTT |
| | | GCTAGGCCAAGACGGCATGACGTTGTACCCAAAAGGGTTTCATTGTATATTGACTATAAA |
| | | CCCCATTCATCTTCTCACTTAAAAGAAGAAGATGACTTGGTTGAGGAGATTTTAAGAGA |
| | | AGCTACAAAAACACTAGAAGGAACAAATCCATATTTGTGATCATTAATCCGTTTGGTGGT |
| | | AAAGGTAAGGCGAAAAAACTGTTTATGACAAAGGCAAAGCCGTTACTATTAGCAAGTCGG |
| | | TGTTCCATAGAAGTGGTTTATACAAAATACCCTGGTCATGCTATAGAGATCGCGCGGGAA |
| | | ATGGATATTGACAAATATGACACTATTGCTTGCGCTTCGGGAGATGGCATTCCTCATGAG |
| | | GTGATCAATGGGTTATACCAAAGGCCTGATCATGTCAAAGCATTCAACAATATCGCCATT |
| | | ACAGAAATTCCATGCGGATCAGGTAACGCAATGAGCGTATCCTGCCACTGGACAAACAAT |
| | | CCTTCGTACTCAACTTTATGCTTAATTAAATCGATAGAGACTAGAATTGATTTGATGTGT |
| | | TGTTCGCAGCCTTCTTATGCAAGAGAGCACCCAAAGTTATCATTTTTAAGTCAAACATAT |
| | | GGTCTCATTGCAGAAACTGACATAAACACTGAATTTATTAGATGGATGGGACCTGCAAGG |
| | | TTTGAATTGGGTGTAGCCTTCAATATCATACAAAAAAAAAAATATCCTTGTGAGATATAT |
| | | GTAAAGTATGCTGCCAAATCAAAAAACGAGTTAAAAAATCATTACCTGGAACACAAAAAT |
| | | AAAGGGTCGTTAGAATTCCAGCATATTACTATGAACAAAGATAACGAGGATTGTGATAAT |
| | | TACAATTACGAAAATGAATACGAAACCGAAAACGAAGATGAAGATGAAGATGCGGATGCG |
| | | GATGACGAAGACTCCCACTTGATATCTCGTGATCTGGCAGATTCTAGTGCTGATCAAATT |
| | | AAAGAGGAAGATTTCAAAATAAAATATCCATTAGATGAAGGTATCCCTAGTGACTGGGAA |
| | | AGATTGGATCCTAATATTTCGAACAACCTAGGTATCTTCTATACGGGTAAAATGCCATAT |
| | | GTGGCTGCTGACACTAAATTCTTTCCGGCAGCGCTTCCTTCAGATGGTACAATGGATATG |
| | | GTTATCACCGATGCAAGAACCTCGTTGACGAGGATGGCACCAATCCTGCTGGGACTAGAT |
| | | AAGGGTTCCCATGTTTTACAACCGGAAGTCTTACACTCTAAAATTTTGGCATACAAGATA |
| | | ATACCAAAGCTAGGGAACGGCTTGTTCTCTGTCGATGGCGAGAAATTTCCTCTAGAGCCC |
| | | CTTCAAGTCGAAATTATGCCACGCTTATGCAAGACGTTACTGAGAAATGGCCGTTATGTG |
| | | GACACAGATTTCGATTCTATGTGA |
| YGR101W | PCP1; | ATGTCAGGTGTAAGCTCTGTTATGCTCGGTCTTCGACCTGCTACAAGAATTTTTTTCCGC |
| | MDM37; | AGTAATATTTCGGTTTCACCTTCGAGGACTTTTGTATCATATATTGGAAGATCCCAGAGC |
| | RBD1 | ACGTCGATACTCAAAAATGCTCCCAACTTAGAGGACAATGTCACAAATCTTCAGAAAATT |
| | | ATACCGAAACGGTTCTTTTCTCAAACATCAATTTTGAAATCAAGGTGGAAGCCTATATTC |
| | | AATGAAGAAACTACTAATCGATACGTACGTTTGAACAGGTTTCAGCAGTACCAGCAGCAG |
| | | AGAAGCGGCGGCAATCCTCTGGGCTCTATGACTATTTTGGGGCTCTCTTTAATGGCAGGA |
| | | ATATATTTTGGCTCCCCTTATTTGTTCGAGCACGTTCCACCCTTTACGTATTTTAAGACG |
| | | CATCCAAAGAATCTGGTATACGCGTTATTAGGCATCAATGTTGCCGTATTTGGACTATGG |
| | | CAGCTACCCAAATGCTGGAGGTTTCTACAGAAGTACATGTTGCTGCAAAAGGATTACGTA |
| | | ACTAGCAAAATTTCGATAATCGGAAGTGCGTTTTCACATCAAGAATTCTGGCACTTAGGT |
| | | ATGAACATGCTAGCGTTGTGGTCCTTTGGTACTTCACTCGCAACAATGTTGGGAGCATCC |
| | | AATTTTTTCTCCTTATATATGAATAGTGCCATTGCAGGTTCTTTGTTTTCGTTATGGTAT |
| | | CCAAAACTGGCACGCCTAGCCATTGTCGGACCTAGCTTGGGTGCCAGTGGAGCGCTATTT |
| | | GGGGTTTTAGGATGTTTTTCATATCTATTCCCACACGCTAAAATTTTGCTGTTTGTTTTC |
| | | CCAGTCCCAGGTGGGGCTTGGGTAGCATTCTTGGCTTCAGTGGCATGGAATGCAGCTGGT |
| | | TGTGCTTTAAGATGGGGGTCATTTGATTACGCTGCGCATTTAGGTGGCTCTATGATGGGG |
| | | GTCTTGTACGGATGGTATATAAGTAAAGCTGTAGAGAAACAAAGGCAGCGTCGCCTTCAG |
| | | GCTGCTGGTAGGTGGTTTTAA |
| YPL118W | MRP51 | ATGACATTGGCAGAGTTGCTTGGACGGTCGAGAATAGCTCAAGTAGCTAACAATCATAAA |
| | | CCATTGACATACACTGGCAAAAAATTTCATCCAACACATCAAATCATCGAAACTAAGCCA |
| | | TCGACACTATACAGACAAGAATGGGGGTTGAAATCCGCTATTCCTTCGAAAATTAAATCT |
| | | CGATATTTGGTGTACAACGATTTGGATACGTTGGAAAGAATTACCACATTCGAACCTAGA |
| | | GGCGGTACGCAATGGAATAGGCTAAGATTTCAAGAAATGGGAGTACCAATTGTGTCTAAT |
| | | ATAGGCAGACAAAATCCATTTTTCAAGTACATATCTCGTCCTGAGGATGAGTCTCATGCT |
| | | AAATTGTCCTTATTCAAAGAAATGAAAGGAGATACAGATATTTCGCCTGCTGCTATGAAG |
| | | AAGCGTTTGAAGAAGATAACAGCACTAATTAGATCGTTTCAAGATGAATTTAAAGAGTGG |
| | | CTGGTGGAAAATCATCCGGATGAATTGAAATTGAATTCTAATAAGCTGGAAGACTATGTA |
| | | GTGAAGTTTCTAAACAAAAAGTTGGAAACAAAAACGAATAAAAAATTTAACACTGAAATC |
| | | ATTGGTACAGGTGGATTATCATACAGCTTGCCAGGAAAGCTAAAAAACTCACCAAATGGT |
| | | GTCATTCAAAGGACTGTAGTACCCGGTAGAATCTTGAACGTTGTTAAGGAGAACAACGAC |
| | | AATAAATGGCTGGCAGCTATTGGTGGGTTTGTGGCCGATGTGGTATTTTTCCAATCACCA |
| | | CCTAGTTCCTTTAACTCCATGGGAGATTTCATCAGAATGAAAACGTTCTTGTTCGAGATC |
| | | TTGGAGGCATCTATGGAGAAAAATGGTTCTGTTTCGATGCACGCTAGATTACTCGAACCA |
| | | CAAAATGACAAGACCAGAGAATTTTTCAATAAGAGACCAATTTATAAACCATTGACCTCT |
| | | AGAAGGGCACGTCGACCATCAGTGGGAAACATTCAAGAGGCCAATAACCTTTTGAATATA |
| | | ATCAAGGGAAATTGA |
| YOR359W | VTS1 | ATGAAACATCCGTATGAGGAATTCCCCACAGGTTCCAAGTCACCATATAACATGTCTAGA |
| | | GGAGCTCATCCAGGCGCCGTTTTGCTATCACCACAATCTTCTGCCATAAACAAGAACAAT |
| | | CCGGGCAGCAATTCTGGCAATAACCAGGGCAATTCATCCGTGACCGCGAATGTTTTGTCC |
| | | CCTCAATCCCATAGTATGTCACTCAATGACATGCTTGATCAGCAATCCTTTATGCTAGAC |
| | | ACTGCGGGAACCAGGGCTCAGCCGCTCCAGCAACAACAGCAGCAGCAACAACAGCAACAG |
| | | CAGGCGTCGTTGCCTTCCCTTAATATTCAAACAGTTTCGTCTACAGCAGCTGGTTCTGCT |
| | | ATCGTTTCCCCCATGATGCAATCTCCAAAGGCGTTGCAATCTACTTTATCTTCAACATCT |
| | | ATGTATTTGGATTCATTTCAGAGATCTCCAAATAATATTTTGGGTATTCCATCGCAGAGT |
| | | GGTTCAATTCCTTTGCCGCAATCCCGTCAATCGCAACAACAGTCTCAATCCCAAAAAAAC |
| | | GATCCGAACATGGGCACCAATTTCAGCCAAGACATCAACCAATTATGTTCTTGGATATCC |
| | | ATGCTGAACAGCAGCCAACAAAATACTGTAATGGACAATATCCTTTCCATTTTGAATGAT |
| | | GATGTTTTGAAATATACTAAGTTAAAAATAGAGACTTTGACAAACACTCCCTTCATCTCC |
| | | CCACCGCTACCTGCAATTGCATCACCTATACCTAATAGGGATGACACACAAATTTTGAAT |
| | | ATTGATTCAGTCTTTTCTTCAAGTCCTATAACTAATGACCCTGAAAATACCGATAACTTG |
| | | TTGTATCAAAATTGGTCCCCTCAGCCACATTCAATACCAATTAGCCAACCCATATATGAC |
| | | AACATCACCGACGCTAGTCAAAGGTCCAAGTCGGCTGAGCCTCATGTTAATTCAAGCCCA |
| | | AATCTCATTCCTGTCCAGAAACAATTCAACAACGGTAACAGTACCAAATATAAAAAGTTG |
| | | CCATCAGAGAATCCCAACTATCTTTCTCACTCTCTCTCAAGTTCCCATTCTTTTTTCCAA |
| | | CCAAAGAAAAGGTCTAACATGGGAAATGAGTACAATTCTCATCACCATCATTCATTGCAT |
| | | CATCCTCTGCATAATACCACCTCATATTTTTCGAATACTTCAAGACCATCAGGAACTGAT |
| | | TTGAATAAGTCCAACCAGAACGTGTTCAATAATACCATTACGCATCCTAATGCTGGTCCT |
| | | ACAAGTGCCACATCGACATCTACATCATCTAACGGAAACACTCCTTTATCGAGTAACTCT |
| | | TCGATGAATCCAAAGAGTTTGACAGACCCCAAGCTACTAAAAAATATACCCATGTGGTTA |
| | | AAATCGTTAAGACTACACAAGTATTCAGATGCCTTAAGCGGAACCCCGTGGATCGAATTG |
| | | ATTTACTTAGATGACGAGACTTTAGAAAAAAAAGGCGTTCTTGCCTTGGGTGCAAGGAGA |
| | | AAATTGTTGAAGGCTTTTGGAATCGTTATTGATTACAAAGAACGTGATTTAATTGATAGA |
| | | TCTGCTTATTAA |
| YBR120C* | CBP6 | ATGTCTTCTTCCCAGGTCGTCAGGGATTCTGCCAAAAAATTAGTTAATTTACTGGAAAAA |
| | | TATCCAAAGGATCGTATACACCACTTGGTCTCATTCAGGGATGTACAAATAGCAAGATTT |
| | | AGACGTGTAGCGGGTCTGCCAAATGTAGATGACAAAGGAAAATCTATAAAAGAGAAAAAA |
| | | CCCTCATTAGATGAAATAAAAAGTATAATTAACAGAACTTCCGGTCCATTAGGACTGAAT |
| | | AAGGAGATGTTAACCAAAATTCAAAATAAAATGGTAGATGAGAAATTCACGGAAGAAAGC |
| | | ATCAACGAGCAAATTCGTGCCTTGAGCACTATAATGAATAATAAATTCAGAAACTATTAC |
| | | GATATTGGCGATAAGCTCTATAAACCTGCAGGAAATCCCCAATATTATCAACGGTTAATA |
| | | AATGCCGTTGACGGTAAGAAAAAGGAAAGCTTATTTACTGCAATGAGAACTGTATTATTT |
| | | GGTAAATAA |
| YKR046C* | PET10 | ATGTCTGAATCATCTATTTCTTCTTCTAAACCATCTGTGGAATTGCCACAAGCAACCTGG |
| | | TCGCATCTGCAAAGATACCCAGCTTTATCCAAGTTTATTAAATATGCGGAATCTCTGCCA |
| | | CCTGTGGAGAGATTGATTTCCTTCAACCTCGTTGTTTTGGGATCTGTGAACCAGTGGGTT |
| | | TCCGAATCGTCCAGCTCTCCTCGTCTGGTGAAGCAAGTTGTTGCTGCTGGGAAGGAAGGG |
| | | GCCTTCAAGTTGGACGAGTTAGTTAACCTCTTGGTGTTCAAGGAGGGTGTCGACGGCTTG |
| | | CTGTACAATTGGAAATCACATTCCAACACGCCAGGGATCTGGCTGGTGTGGTTCTTCGTC |
| | | GACTACGTCGCCAACATTTCTAATACTCTGTTGAGGGAGTTCCTGATCAAGCCATTGCAC |
| | | TTGCAAGGTTCTACCGCATCGAAGGAGATCGGCTCTTCCGGTGAGGAGAACAAGGTCACT |
| | | GATGCTTCTTCTTTGCCCCACGTGGCAGAGTTGTCTTCAACGACCAGAGGTATGTCGCAG |
| | | GAGATCCAGTCCAAGGTCAAGTCGAACTATATCGACCCAACCAAGGACCTGGCTAAAGAA |
| | | AAGTACGACGCCATAGTGAAGCCCACAACTGACAAGTTGCAGTCTGTGTACATCGACCCA |
| | | ACAAAGACTAAGCTTAACGAAACCTACCAACGCTTCACCACTGTCTATGAAAACAATCTA |
| | | AGTAAATCTGAAAGCGTCCCTAAAGCCATTGTATCCACCGGGTTGGACTTGGGCAATGCC |
| | | ACCATTGAGAAGCTAAAGGCCTCAAGAGAAGACCAAACCAATTCTAAGCCCGCGGCTGTG |
| | | TCGACCAATTAG |
| YPL219W | PCL8 | ATGGCTAATGATCAAGATCCCAACAAGTCTCTCATTAATGACGCTTTGACTCGGAGTATG |
| | | TCCGAATTTTATGATGATGATGATGATAACGACAGTGACATGTGTAGGGCAAATGATGAG |
| | | GGAGAAGACGTTTTTGACCTTCCACTTAAGGTAGGCGTTTCTCAAAGCAGGAACTTTTCA |
| | | GAAGTAAATGACGTGTTAGACCCGCTTTCTAGTTTGCATGGTCCGTCCAAGAAGGTGAGA |
| | | TTTGAACAGCAAAAGCAGCAACAGCAACATCAGCAACTTCACAATGACTTCAATACAGAT |
| | | TTCAACCTGAAAAGCCCGTCCAGTAAAAAAATGGGGGTAGAACAGCTGATCCAGTCAGCC |
| | | AATGAAATCAACGATTACCTCGCTAACAACATTGACAAGGTAAATAGCTTTAACTCAGAG |
| | | CTATTGAGCGGCAGTGGCAAGTTACCTGGGAGAGTAAAGAGTGACACAGCAACTCAAGGT |
| | | ACCGGACGCTTAGATTCCATGTCCAACTTTGCTCTTAGCGATACGGAGTTGGACAATGAT |
| | | GACGATAATTATCTCCTGGATCCCCTTGCAAATGCTAGCTCTACTACACCCACTGTGGAA |
| | | CACCACGGTTACAGCTTATTAGACAAGGCTTTATCCACGTCTGATAAGGAAAAAATATAT |
| | | ACGAATAAAGTGAATTCGAATAGTCAGATAGATACTGATAATCACTCTCACGAAAGTGGA |
| | | AACACTACCAATAACGAAACAGATGAAAATGAGAGTTCGGAGATCCTGGACTACACAAAA |
| | | TTTGATTCTTTCCCCTATCCCCCGTCTTCTGCCCCCAATGGGGAACCACCAGATTTGAAA |
| | | GTGCTAAGCATTGAATGTGAGCAGGAAAATGAAAAAGAGCTACGACGAATATCTTTATTA |
| | | TTGGATCATTACGAATCAATACCGAAAATACCAGAATTGTCAGACGATGAAGCACTATCT |
| | | AAGTTCCGTGAGAATATCGAATTAATTTTACAACTATCTAAGAAAATTAACGATAATGCG |
| | | AACACTTTAGCGATTTCCTCCGAAGATCCTCAAAAATTTGTTAATTTTGTGATGAAGAAC |
| | | CCACCTTCCTTATCATTCAGAGATTTTATCGACAGAATCCAGAATAAATGCATGTTTGGT |
| | | GCCGTAGTCTATCTTGGAGCCACCTATCTACTTCAACTTGTGTTTCTTACGAGAGACGAA |
| | | ATGGACGGTCCCATAAAGTTGAAGGCTAAATTACAGGAGGACCAAGCCCATAGGATCATC |
| | | ATATCTACAATAAGAATTGCCACCAAGCTATTAGAAGACTTTGTCCACTCACAAAACTAC |
| | | ATTTGCAAAGTCTTCGGTATTTCAAAAAGACTGCTGACCAAACTGGAAATTTCTTTCATG |
| | | GCGAGTGTTAATTTCGATGGATTAATGATTACTTGTGAAAAATTAGAAAAAACATTGCAT |
| | | ATCCTTGATGATACAAGACAGGCATTAGGAAACACATGA |
| YMR187C* | — | ATGACCCCACCTCATTTTTTTTTATCTCTCATAAAAAAAAGATGCATATGCTGGATATGT |
| | | TTGGAAGAATCCACATATGACTCTACATGGCTACAGCACACGTGCGGTTGCAACTTACAA |
| | | ATTCATAAACGCTGCTATATAAGATGGCTCTATCAAATGCATGTTGAACTTTTTCTTCCG |
| | | AACACAGTGGACCTACCAAAGGATGCTGATTTACCTATAATCACCTGTTTAAAATGTCTC |
| | | GTTGATGGGCACCATGATTTTATGACGACTTTCTCTCTTACAGAAATATGGGAAACTCGT |
| | | CCCATTTGGGGGCAAAAATCAGTACCGTTTCAAAATGACTACGTGTTCAATCTAATGTCT |
| | | CTTTATACCAAACGGGATAATCATCCACCTTATGTGTTAGTGAAATTTGGCGAATGTCCT |
| | | CAATGCAAGAAAACAAATTTCATAAAAAGACCTACAGTAACCATCCAGTCTTCTGTACTA |
| | | TCCCTGTTTTATCAGTGGCAAAAAATTACACGATACGTGATACCATTAGGGATAACTAGT |
| | | TTATTTCTTTTAAACCCTGAAAAGACAAGTTTTGACATTGGATTATGGCAACTACGCTGC |
| | | CTTTTCCCTGAGAATGTTCTACGAAATATGTTAAACATCAGCACTACAAAGGCATTGGAC |
| | | GTATATGCGCAAACTGAACGTGGCTTATTAAGCATTCCTTTAACCTCCTCGATCATAATA |
| | | TATGGGTTCATACATTATCTTAGCAACATTTCTAACGTCAGTGCTAATGCAATACTCTTT |
| | | AAGTGGGTGTATCTCAGTATTGTAAAAACTGCTGGAAACAAATATTACAAGGGTATAGGT |
| | | TTACCTAAAATAATTTTGTATTCTAACTTGGCTACATTTTGCTATAATTTTACCTTTAAA |
| | | AGATTGGTTGACTTAATTTACAGGAGGCTGATCAATAAAGGAGGTAAGTATTTGTATCAC |
| | | GGAAATTTTGAAAACTCTAGTAATAGCGTTCCAGCTGAGGAATTTTTTATCAGGCGGAAT |
| | | TGGTATGCTATTCTTGCAGAAAAGATACTATGGCCCTTTGTTGGTAAATGTACAGGAGGT |
| | | CTTCTATTGAATGCTTTTCTATGGATACAGAGAAAGTTTAAAATTGAATGGACGCCGAAT |
| | | TGCTCCCCTTCGGAATTTAGAATGATTTTCAATATTATAGGTTGTGGTACTGCTGCTATA |
| | | GGCTGGTCAAGTCTGAAGCTCTATGCCAGTTACAAAAGATGTCAGGAACTTGAAAAGATC |
| | | AATGAATTTATTGAACAAAGTTGCAAAGGTGAGTAA |
| YGL139W | FLC3 | ATGAGATTTCTTCAGGTTTATAAATCATCGGCATTAATTGGACTAATAATATTGTTAGCC |
| | | AGTAAAGTAAATCTTGCAGAGGCAAAAAGGAAGCTGGTAGCCACATCATTGGTCACATGT |
| | | ATGGAAAACTCGCAGCTGTCAGCAAATAGTTTTGACGTTGTTTTCAATCCAGACGACAGG |
| | | TCATTACATTATGATCTTGATATGTCAACTCAAATTGATTCATATATTTTTGCTGATATT |
| | | GATGTCTACGCATATGGTTTCAAGATCATTACTAAAAATGTCGATCTTTGTTCAATCAAC |
| | | TGGAAACAGTTTTGCCCGGTTCACCCAGGTAATATTCAGATCGACTCTATTGAATACATC |
| | | TCCAGTGAGTACGTAAATGAAATTCCTGGTATTGCATACCAAGTCCCCGATATCGACGCA |
| | | TATGCCAGGGTCAAGATCACCAACAATGTGAGCGAGTACCTAGCATGTATCCAAATATAC |
| | | TTCTCAAACGGTAAAACAGTTTCTCAGATTGGTGTAAAATGGGCAACTGCAGTGGTAGCG |
| | | GGAATTGGTCTTTTACTATCTGCCATCTTATCCACTTTTGGGAACTCTACCGCAGCCTCA |
| | | CATATTTCTGCCAACACAATGTCACTTTTCTTATACTTCCAATCTGTTGTCGTGGTTGCC |
| | | ATGCAACATGTTCATCGAGTTCCGCCTATTGCAGCCGCCTGGGCGGAAAACTTGGTTTGG |
| | | TCAATGGGGTTAATTAGAATCTCATTCATGCAAAGGATTTTCCGGTGGTATGTGCAATCC |
| | | ACTGGTGGTACGCCTAGCTTATATCTGACTTCAACATCTATGTCTGTTTTGGCTCAAAGA |
| | | AGTTGGCAATATTTGATGGAGCTCCCATTAATCAAAAGAGCTACCAATGTTTTGTATGGT |
| | | AATGCAAATACATTAATTTTTAGAGGAATCAAAAGGCTAGGCTATAAAATGGGAATCGAA |
| | | AATACGTCCATCGTTTGTACCGGTTTTACTTTCTTCGTCTTATGTGGATATGTGTTGGCT |
| | | GGATTTATCATAGTATTCAAATGCTGTGTGGAGTTAGCCACCCGGTTAGGATGGATCCAA |
| | | AAGGCAAGGTTTTGGGAATTCAGGAAACAATGGAGAATGATCTTAAAAGGTGCTCTTTTA |
| | | AGATATATTTACATCGGTTTCGTTCAGTTAACAATTCTCAGTTTTTGGGAATTTACTGAA |
| | | AGAGATTCACCTGCTGTTATCGTCATCGCATGCCTTTTTATTCTTTTGTCATGTGGGTTA |
| | | ATGCTTTGGGCTGCATGGAGGACTGTTTTTTTCGCAAGAAGATCTGTTGCATTGTACAAC |
| | | AACCCAGCAGCCTTATTGTATGGAGATGAATATGTCTTACACAAATACGGATTTTTCTAT |
| | | ACAATGTTCAATGCCAATCATTACTGGTGGAACATTGTTCTTTTATCCTACATTTTTGTA |
| | | AAATCATTATTGGTTGGATTTGCTCAGGCTTCTGGTCAAACACAAGTTCTTTTCATGTTC |
| | | ATATTGGATTTGTTTTACTTCGTTGCTATTATCTACTATAAGCCCTATCTGGACCGTCCA |
| | | ACAAACATAATGAATATTTTAATTGCTACTGTTACTGTAGTTAATTCCTTTCTATTCATG |
| | | TTTTTTTCGGACTTGTTTAACCAAAGCTACAAAGTGGCTGCTATTATGGGATGGATATTT |
| | | TTCATTATGAACGCTGCATTTTCGTTCATTTTACTAATGATGATATTGGCATTCGCTGGT |
| | | ATGATGCTTTTCTCAAAGAACCCCGATCTAAGATTTAAACCTGCTAAGGACGATAGAACT |
| | | TCTTTCCAAAGGAACACTATGAAACCAGAAGGTACTGTTAATAGATCAGTAGCGAACGAG |
| | | TTGCTTGCGTTGGGCAATGTGGCAAAAGACCATGACGACAATTCAGATTACGAATCAAAT |
| | | GATACTGGTGTGAATGATGAGCTAAAGCAAGCACAGGATGAAACCACTCCCACAACCGTA |
| | | ACATCAAGTGATGATAACAAGCCTACGTTTTCAGAGAAAATATTAAGTAAATTCTCTAGG |
| | | CCTAAGAATGAAAACGCAAGCACAGATGCTCTGAGAGTTGAAGCTCCAAAACAACAAACG |
| | | TTTCCTCACAACTTAACGAATCTTTCACGAGAAAACCTATCAACATTAGGCTCTAAACCT |
| | | TATCCAGGACACACAAGATCACAGTCTGATGCGCATAATGGACTGATCAATTCCTTTGAA |
| | | GAGGAAGATACAAGTTCAAATACCGATCCATTTCACGACTCCACTGAAGGAGACCTTTTA |
| | | GATACCTCAAGTTCGGACGGAGGATTTAGGTCGCAAAACTATGTTCGAGATGACAGCATA |
| | | AATAGTTTGGGAAACAATAAACAACCACTGAGGAAACCCCCTGGATTCTTCGATGAAGGA |
| | | TTTATGTGA |
| YLR199C* | PBA1; | ATGCTTTTTAAACAATGGAATGACTTACCAGAACCAAAACATCTGCTAGATCTCCCAGAG |
| | POC1 | ATTTCAAAAAACCTGCAATCTTTAGAAGTCTGCCCGGTTCCAAAAGTTGAGTTTCCTCAG |
| | | GACTTGGATGTTCCTCAATATTCGACAGCCGTCATTACTACTAAGATAATGAACCCATTA |
| | | TTCCCCAAAAATCTGCTTCAATTAACCTCTATTGGTGAAATTAAAACTACATTGACGGTG |
| | | AAAAGTCCAAGTCTTCCCCAATCTTCAGGTAAACACTCGTGGAATTACGACGAAAACTTT |
| | | CCAAACGAGGTTGATCCTGATCAAAAAAATGACACCGCCGATGAAACAGTATATGGTTTT |
| | | TCTTTCCCGATTTATTCATTTGGAAAGACGTTATTATTTTCTATGGAGGAGAATTTCATT |
| | | AGTATATCGCCGATTTTTGGTAACATGATAAGTAGATCTATCATATCACAGTTAGCCCAG |
| | | TTCTCCCCTGATATAATAGTAATTGGCACCTCTGATAAAATCGCCAGCATGAAGGTAATG |
| | | ACGGAAAATGAATGTACACTACAACCACCAGAATTTATAACGGGATTCATAGGAAGTGTG |
| | | CTAACACAATTGATTGTCGGTCCCAGTAAGGGACTGAAATTTAAATGTCTTGTAGCGCCT |
| | | AGTGAGGGGCCTAATGGATTTGAGAAACTTTCGTTGTCTGACATGGGTTCTTTAGTTGAT |
| | | TTGTGCGGTCAATGGCTTGGTTTTGAGCCTTCAAGATATTCTGAAGAATGCTATCGCTTA |
| | | TGGAGGTGTGATAGCGCGGCAATTGGTGCACAATCAGGCCTATATATATGA |
| YGR241C* | YAP1802 | ATGTCTTCATTATATACTAAGTTGGTTAAAGGGGCGACCAAAATAAAGATGGCCCCTCCA |
| | | AAGCAAAAATATGTGGACCCTATTCTCTCGGGCACTTCGAGCGCAAGAGGATTGCAAGAG |
| | | ATTACCCACGCATTGGATATAAGGTTGTCTGATACAGCCTGGACCATTGTTTATAAGGCA |
| | | TTGATTGTGTTGCATCTGATGATACAACAAGGCGAAAAAGATGTCACGCTAAGGCACTAT |
| | | TCCCATAATTTGGATGTTTTCCAATTGAGAAAGATTTCGCATACTACTAAGTGGTCTTCC |
| | | AATGATATGAGGGCTTTGCAGAGATACGACGAATACCTAAAAACACGTTGTGAAGAGTAT |
| | | GGCAGGCTAGGTATGGATCACCTACGAGACAATTACTCCTCGCTAAAGCTGGGCAGTAAG |
| | | AACCAGCTTTCAATGGATGAAGAGCTAGATCATGTTGAATCATTGGAAATTCAAATTAAT |
| | | GCCTTGATAAGGAATAAATACTCTGTTTCAGATTTAGAAAACCATTTGTTATTGTATGCA |
| | | TTCCAATTGTTAGTACAAGATTTACTAGGTCTGTACAATGCTCTTAATGAAGGTGTCATT |
| | | ACACTTTTAGAATCATTCTTCGAATTGTCCATTGAGCACGCAAAAAGAACACTGGATCTC |
| | | TATAAGGATTTCGTGGATATGACAGAATATGTTGTTAGATACCTAAAAATAGGAAAAGCT |
| | | GTCGGTTTGAAAATCCCCGTTATCAAACATATTACTACAAAGTTAATTAACTCCCTTGAG |
| | | GAGCATTTGAGAGAGGAAACAAAAAGACAAAGAGGTGAACCTTCTGAACCGCAGCAAGAT |
| | | AGAAAACCATCTACGGCCATTAGCTCTACCAGTAGTCACAATAACAACAGCAATGACAAG |
| | | AATAAGTCTATAGCGCAGAAGAAACTGGAACAGATAAGGGAACAAAAAAGACTGCTGGAG |
| | | CAACAATTACAAAACCAACAGTTGCTCATTTCCCCCACGGTCCCACAAGATGCTTACAAC |
| | | CCATTTGGATCACAACAGCAAGATTTAAATAATGACACCTTTTCATTCGAACCAACCCAG |
| | | CCACAAATGACCGCTCAAGTCCCTCAACCAACTGCTAACCCCTTCTTGATACCGCAACAA |
| | | CAGCAACAGGCTTTACAGTTGACATCTGCGTCTACAATGCCACAACCAAGTGAGATACAG |
| | | ATTACTCCAAATTTGAATAATCAACAAACTGGGATGTATGCTTCTAACCTTCAATACACC |
| | | CCAAATTTCACCGGCTCAGGGTTCGGCGGATACACGACCACCGAGAACAATGCAATTATG |
| | | ACCGGCACTTTAGATCCAACAAAAACGGGGTCTAACAACCCATTTTCGCTAGAAAATATT |
| | | GCCAGAGAACAGCAACAACAAAATTTCCAAAACTCCCCAAATCCTTTTACCCTCCAACAA |
| | | GCACAAACCACCCCGATTCTTGCTCATAGTCAAACAGGTAATCCATTTCAGGCTCAAAAC |
| | | GTGGTGACATCACCAATGGGCACATACATGACGAATCCCGTGGCCGGTCAACTTCAGTAT |
| | | GCCTCTACTGGTGCGCAGCAGCAACCACAAATGATGCAGGGTCAGCAAACAGGGTATGTT |
| | | ATGGTTCCCACTGCGTTTGTCCCCATCAATCAACAACAGCAACAACAGCAGCATCAGCAA |
| | | GAAAATCCTAACTTAATAGATATATAG |
| YGL110C* | CUE3 | ATGTTATCAAGGTACAACCGCGTTATTGAAATAAATGGGGGTAATGCAGATATTTCTCTG |
| | | CCCATTGTCAAATTTCCACCGTTTAAACTCAGAGCTCAATTAATTGAAAAGGACCCCGTA |
| | | GTATGGCTGCATCTAATAGAAACCTATGTAACGTATTTTGAGTATTTGATGCAAGGTGCT |
| | | AACGTTGAATTATTAGATGAATCTACACTTGATCATCTTCGTTTATTTTTGAGAACTTAC |
| | | TTGCATGAAATTGCTGATGAGGAAGGAAAGTTATTAAGTCTCGGTATCAACCATGATGTG |
| | | TCTGAACAGTTATATTTACTAAAAGGCTGGATATTTTCATTGATCAAGAAATGCGGGTTA |
| | | TTACATCTTCAGATTTTTGGTGACTCCCTGTGGAATTTGATCAAAGTATATGTTAGACGT |
| | | AATCCAGATTCTATTCGTGGTTTGATCGATGGCTCATTGAAACCACGGATCAACACGCAA |
| | | CGTGTACAATTGGACAAATCGTACCAAGTTCAACAGCATTTGAAGCAGTTAATAGAATCT |
| | | GGAAAATTTAAAAGAATAGATTTAAGGTGCGTTGAAGATCTGCTGTCTGCCAAATCCATG |
| | | CAGCCAAATAAGTTTGCGGAGAACTTCTTTACGGCAAATTGGATTGAAATTTTAGAGGCA |
| | | TTATGGGCAAAGGGACAGGGCCGGGGTCATAAAGAGGCTAGAGAATTGATCATTATCAGT |
| | | TTATTTTCTGTTTCTGCTGATCGTTTACTAAAAATAACTAAAGAATTAGGCATAAGCAAT |
| | | TTTGAAACTTTGGCGTTATATCCTCTTTTAGGAACAATGCTCATAAATGAAGGAGTCCAC |
| | | AAAAGGCTTCCTGATTTGAAGAGCAAGTTACTATTTCTGAATTTAGGGGGGTTGTCAATG |
| | | GACGAAGGAGACCATATGAGCTATCCAACATCATCAGGTACTGAAGTTAATGAAGAACAA |
| | | CTGTCCGCGTTGATGGAATTGTTCCCCCAGTTTTCTAAATATCAATTATCTCAAACGTTG |
| | | CTTGCCTATGATAATAACATTGAATTAGTAACGAATAAAATATTTGAGGATCCTACCATT |
| | | ATCGAAGCATTCTCCAGAGAACCTGCGGAGGAAGAAGTAGAGCCGGTGTCAGATGGTGAT |
| | | AATGCATCCTTTACTGAAGAACTGAGTATATTGGATAGAGGAGATAGCTCAAAAAATAAA |
| | | GAGCTAGACAAAAAGATTATCTCGGAGGGTGTTCCGGATGAATTAAGAAATAAAACTTTG |
| | | ACAAGAGCATTGAAACTTTTATACGAGGCTGATGAAGATGAAAGGGATGACACTTATGAT |
| | | GAAGCCGATGTGAACCGGTCCGATCCTTCGAAAAGAATTGGACTTCAAGAAGACGAAGAA |
| | | AGCTATGATACAAAAGATGATTCTAATGAAGTGCGACAAGATCATAACTATCATATTGTT |
| | | GAAGCTTACTTGTGGAATCTATTGAAAGAGGATCCGAAATTATTTGAGAGATCAAAAAGA |
| | | GGTACAAAAGTCAGGAAAACTATGAAAGAAATGACTTCATGGTCGGATGAAAAAATCGAA |
| | | GGTTGGTGTCGGATGTTAGAAAGATCTCCTACCCGAGCAAGACTTTTAGAAAAGAAGTTT |
| | | ATGTTCAAAGGAAATAGCAAAACAGGGAAAACGTCTTATGTTCATAATCGTGATAGCCAA |
| | | AATGATGGAAATGTCGTCAAAGAACAAGCCAAACAAAAAAAATCTGAAAATATTAAGAAA |
| | | CATGAACCTCAATCTACTGAACAGAAGAAAAGACAGCATGCTAAGAATGAAAAAAGGAAA |
| | | GGGGCTAGAGCTAACCATAACCGTAAGAAGGGGCACGATAAAAAATTGGCTCGTGCGGGA |
| | | AATAACGCTATTTGA |
| YOL101C* | IZH4 | ATGGTTTCATTGACTACAATAGAGCAAAGCCCAGTCAAATGCGAGACAACCACTGAGAAA |
| | | GAGAGCAATGATACTAGGGGGACAGATTCGAACGAGAATGCAGAGACAAAAGAAACAAAG |
| | | AAGGGTTTTCCATTCCATGACTTGGCAAAATTGCAGAAACAGTACAAAAATAAATCCAGT |
| | | AGGAACGAGTCACTAGTAGCACTAATATATCTCTTGGGTTCGATGCTGTCATTTTGTCTC |
| | | CTAATATTTTTCACGGACTTTTATTTGATACCTTTGTTCCCCACCACCACTACAATGACA |
| | | GACTACATTGTATTCAATTTTTATTTATTGAACGTTTTTGTATTTTGTATGGTGCATTTC |
| | | ATTTATCACTTCGTCAAAAATATTTCTCTACAACAACATCTCGAACATTGGCAGAAGTTT |
| | | TCGTATTTATCGAATATCAACTTATTGATATCATCACAAATCACAATTCTTTATTATCTA |
| | | TTCTATGATTACGTGTTTTTCTTCAAAATTTTTACCCTTTTAATGAACTTCATAGGATTA |
| | | GTGGCATACTTTTTTATATTGACGGACAAACTCATTTCCTCGAAAAGATTCAATAAAACA |
| | | GTTTTCTTCATTTCGGTATCAGTAGTGTGTTGTTCCTTGCCACTATTAACCGCAATAATC |
| | | ACCTTTGATGGTTTAGAAAACCTGAAAGAGAGGATAAAAGTGAACGCAATTACGTGGGAA |
| | | CTTGTGGCTTTGGTAGCGGCATCTATAATTTACGTTACCAGGTTCCCTGAATCATTGTTC |
| | | AGACGAAATAAAAAGGAAGAAGGTTGGAATCATTCCGAGTACCTTTTCCATCTCTTGATT |
| | | TCTGGTACGGCTTTTTACCATTTTTTCATTTTGATTCAATCTTATATTTTGATGCATTCA |
| | | AGTTTGAATCAGCCCGAATTAATTAATTTCAAGTCTTAG |
| YDL173W | PAR32 | ATGGCTACGTTCAACCCACAAAACGAGATGGAGAACCAGGCACGTGTACAAGAGTACAAG |
| | | GTCTCCACTGGCAGAGGCGGAGCTGGCAACATCCATAAATCTATGTCCAAGCCGTCTCCC |
| | | GTACTTCTTCCATTGAAATCCAATTCAAAGACAGTAGCAAACAACAATAATAACGGTAGC |
| | | AACCAGGAAAAGGTTCCTCGTTTTGCAATTGGTAGAGGTGGTGCCGGTAATATCTTTCAT |
| | | GATCCGCACCTGACGAGATCCGCCCAACAACTGGACTCTAACGACAATATCAACTATAAC |
| | | GACGTGATCAATGACATCGACGATTATATTTCCCCTATAACTTCGGATATGGTGGATGAA |
| | | GGTGGGTCAAACCCGGTGACCAACACCAGGTCTCGTATCAGCGCTACAAGGAGCCATCAG |
| | | TCTTTGCATGCCACCACTTCGTCTCCGAACAACAACGCCCCAATTGTCGTTGGTAGAGGT |
| | | GGGGCAGGAAACATTTTCTTCAACAAAAAAAAGGTGGCCAGCAATGGTGGAAATGAAGAG |
| | | GATGAGATACGAGGCGGCAATATCGAGGATGAGGATACTATCAATGCAAACGAGGACAAC |
| | | CTGTTTGTGGTGACTTCCAATGGCAATGCTTTGGCGGCAATCAAGTCCACGTCCAAGAAG |
| | | CCTAAAAATAAGCTCAAGGGCAAAAGCGCACCGGAAAAATTTGCTATTGGAAGAGGTGGC |
| | | GCTGGAAACATAATTTCTCCAAAGTCAAGCAGGAACACTATCAACCATAACTTAAACGAT |
| | | GATGATGAAGATAAGTTTAATTTGAAAGATGACAATGGTAAAGAGAAGAAGAAGAAGAAG |
| | | AAGAAGAAATCAGGGTTTTTCAGTTCTTTGAAAACTATGTTTAATTGA |
| YDL054C* | MCH1 | ATGCCTCTATCAAAGGTGGAGCACTACCTTTCATACCATACGCGCTTACTCTTACCCCAT |
| | | GTTTTGTCTCTTCAGTCATCACATCGTGTTGCATACATCTTTTCGCTATTATCTGCGGTG |
| | | TCAACTGGCTTCATTACTTTGATATCTCTTTACTCTCAACCGTGGCAGAAACATTTAAAT |
| | | TATTCCTCATGGCAAATCAACACCATCGCTAGTATGACTAATTTGGGGATGTACTTGACG |
| | | CCACCAATCTTGGGGATGATCGCTGATTCTCATGGCCCCATTACTTTAAGTCTTTTAGCC |
| | | ATCATAGGGTTCATACCTAGCTATTCATATCTGGCTTACGTTTTTAATCATCCGGAGTTA |
| | | TCTCTCGGAGGAAATGGTGACTCATCATTCAATCTATCCATCATTTGTTTCGTTTTCATA |
| | | GGTATATCAACAAGCGCTTTATACTTTAGCGCTTTACTGACATGCACTAAGCTATATCCT |
| | | CATACAAAACTACTATCCATTAGCTTACCAACGACATGTTATGGTATTTCTTCTGTAGTC |
| | | GGTTCTCAACTGCTAAGAATCAAATGGTTCTGGTCCTCTAACGCAAGTTCTTCCTCGTCC |
| | | AATAGTGACTTAAACCTGGGAAGAGTATTCCAAACATTTGCCCTCGTTTATGTCGTTATT |
| | | GGGCTACTTGCATGGATAGCCACCAGCGTGGTATCACTTTTGCATTTTAATGAAGAGCAA |
| | | GACAACCAAAAACGGCTGGATGATCAAACTGATGTGGAACAATCACCGCTGTTAGAACGA |
| | | AGTAATCATGTTCAAGAAAAGTTTACGCAGACGATGCTAAGGATCTTTAGTGATCCTGTG |
| | | ACATATATCCTAGCGGTATCAATTTTGTTATCACTTGGGCCCCTCGAGATGTTTATTGCC |
| | | AATATGGGATCACTGACTAACCTGCTAGTCCAATTAGATGCGCCAACCTTATCTACAAAG |
| | | TTGTTATCCACATACGCGCTATCTTCCACTTTTACGAGATTGCTCACAGGCATAGTGGCA |
| | | GACTTCTTCGCCAAGAAAAAAATATCAATTAAATGGATCCTGTTGACTTTCCTTTCATTA |
| | | GGGGTATGTGCACAACTGTTTTTATTGAAAATGACCTCTTCAGCGTCACCCTGGGGGCTA |
| | | GTACCTACAGGATCATTGGTTGGAATTGTATACGGTGGACTTTTCACTGTTTATCCGACG |
| | | CTGGTCCTGTTAGTATGGGGCGAACGCTCATTCGGGACTGTTTACGGTAGCTTACTAATT |
| | | GCACCTGCTATAGGTTCTATGATATTTTGCATGTTGTATGCCAAATTTTACGATTCTCGC |
| | | TGTATGAGTGGCGGAGGAGATCTGCGAAATCCGTCCTGTATTTCGGCTGTCTACAAGTAC |
| | | AGCAGTATCGCATTCGTTGTATCCGCTGTTCTTTCAGCTGTAGTATTTTGGAAATTAAAA |
| | | AGTAGAAAACTCAGAATTTAA |
| YHR014W | SPO13 | ATGGCACCCAGAAAACGCTTTAGGCTACTCGAATTAGGGTCACCAACACATTCTAAAAGG |
| | | AAAGTTCAAAAACCTCTTCAAGAGAAGACCCCAAACTTGCGAGTTTCTCCACTTGCTTTT |
| | | AAAATTGGTAAAGAAATCAAGAATAAGGAAATCAGAAAAACAAAAAAAACAGAATCAGAA |
| | | AATATATTCAATTCGAAACATGTCGATTTAAGACTAGAAAGCCCACATCCAGGATTAAAT |
| | | TTTGTAAGTGATGCTCAACAGTACTCAAAGGCTGGAGATGTTCGATATTTGAAAAATAAA |
| | | TCAAGCAATACCTTGAAAAACGAAAGACAAACAATAGAGCGCCCCTCATTTGATAATTCC |
| | | TTAAGGTTCGAGGACATTGAGCAGCCTCCAAAATCTACTTCAACGCCGGTTCTTTCTCAA |
| | | TCTTCTCAAATAAATGTAGAAAGAGAAGCACCCATGTTTCCAGTTCCTTACTATATTGCT |
| | | CCTTCTCCTATGTATAATTTTAGCCCGTATCAAAATTTCGTTGGAAACCCAACGTTCCTG |
| | | ACGCCAAGTCATAACCCAAACCTAAACTATGCCATTCCCATCCAACGGCCGGAGTTGCTT |
| | | TATCCGAATGTTAACGTATACGATTCGCCATTGTTTAAGAAAACAAGACTGCCCCATCAG |
| | | ACCAAAAGTCTAGATAAAGAAAAAAATTACCAATATCTTCCAATATATCCGGTCTCGATT |
| | | TCTAATAACGGAGATTTTGTTGGCCAAGAGACACCAAGGGCAGCACCAAAGTTAAGTAAA |
| | | AAGAGACTTTCAAATACATTGGATGTTAATTGTTCCGATTATGAGTCTAGTGGACAAAAT |
| | | GCAACTTATAATGATAGTGAGTCTTCCCTTAATTAA |
| YLR099C* | ICT1 | ATGTGGACAAACACTTTCAAATGGTGCAGCAAAACTGAGAAGGAGACCACTACTGCAGAC |
| | | GCCAAGGTGTGTGCTAGCGTGCAGGGCCTGAAGGCCCTGCAGCAGCAAATTATGGATAGC |
| | | ACCACTGTGCGTGGGTCCGTTAATAACACGATGACTCCAGGCGGGATTAACCAGTGGCAT |
| | | TTCCACAATAAGCGTGCGAACAAAGTGTGCACGCCTACAGTACTAATCCATGGATACGCT |
| | | GCCTCGTCGATGGCGTTCTACAGGACGTTTGAGAACCTGTCAGACAACATTAAAGATCTA |
| | | TATGCCATCGACTTGCCAGCCAATGGTGCCTCAGAGGCGCCAGCATTGCAAGTGAACAAA |
| | | ACCAAGAAGATCAAGTCTCTCAGGTTTAAGCATATAGAAGATGACGTAGTAATCCCTGTG |
| | | ATAGAGAAGCGCCCCCCAGCAGAGGACATCAAGTCACACCTGGAGCAGTACGAAAGCTAC |
| | | TTCGTAGACAGGATAGAGCAATGGCGGAAGGATAACAAGCTTCGCAAAATAAACGTGGTG |
| | | GGCCATTCATTCGGAGGATATATTTCATTCAAATACGCCCTCAAGTACCCTGATTCCATT |
| | | GAGAAACTGTGTCTCATATCTCCTCTTGGTGTGGAAAACAGTATACATGCTATCACCCAC |
| | | AAATGGGAGCCCAACACCACCTATCCACTTACGTTTACCGACCCATCGTCCCGATATTAT |
| | | ACAAGAAAGCTGAACGTACCGCGGTTTATCTTTGAAAACCAGCTAAACGTTTTGAAGTGG |
| | | ATGGGACCCATAGGCTCCAAATTGTGCTCGAACTACATCTCCACCGCATACGTAAAAGTT |
| | | CCGGATCAGATATATAAGGACTATCTTTTGCACTCATTTGTCGGGAAGAACCAGACGGTG |
| | | CAACCACAGACAATCAAGGTGTTCACCCATTTGTTTGAAAGGAATCTGATCGCTAGAGAT |
| | | CCGATAATAAACAACGTTCGTTTTTTGAACCCAGCAACGCCTGTCATGTTCATGTATGGC |
| | | GAGCACGATTGGATGGATAAATATGCGGGCTACTTGACTACTGAATCAATGCTAAAAAAC |
| | | AAGGCAAAGGCTAGCTACGTTGAAGTCCCTGATGCTGGCCATAATCTTTTCCTGGACAAC |
| | | CCGCAGCACTTTGCCTCTTCTTTAGTCTCGTTCCTGTCAAAGTAA |
| YLR193C* | UPS1 | ATGGTCCTTTTACACAAAAGCACACATATATTTCCTACCGATTTTGCCTCTGTTTCACGC |
| | | GCCTTTTTTAATAGATACCCCAATCCATACTCCCCCCATGTACTATCCATAGACACAATA |
| | | TCAAGGAACGTTGATCAAGAAGGAAATTTGCGCACAACGAGGCTGTTGAAAAAGTCCGGA |
| | | AAGCTGCCCACATGGGTCAAACCCTTTTTAAGAGGTATAACAGAAACATGGATAATCGAA |
| | | GTTTCCGTAGTGAACCCCGCTAACTCCACAATGAAAACTTACACTAGGAATCTGGATCAC |
| | | ACTGGAATCATGAAGGTTGAAGAATATACTACCTATCAATTTGACAGTGCTACAAGTAGT |
| | | ACGATAGCAGACAGCCGGGTGAAGTTTTCAAGTGGCTTCAATATGGGTATCAAATCTAAG |
| | | GTAGAGGATTGGTCGCGCACTAAATTTGACGAAAACGTTAAGAAAAGCAGAATGGGCATG |
| | | GCATTTGTTATCCAAAAACTCGAAGAGGCGAGAAATCCTCAGTTTTGA |
| YNL011C* | — | ATGAACGTCGTTGTGTGCTCCGGTGGGACTGCCACAAATTCATTGACTCCTTGCTTTTCT |
| | | AATATTTCTATCTTAAAAGGACATGAGCTGACGTATATATTGCCAATATCAGATAATGGC |
| | | GGGTCCACAAGTGAAATTCTACGAATAGTTGGTGGCCCAGCAATTGGTGATATCAGATCT |
| | | AGAATAGTGAGACTACTTCAAGATGAACAGCTTGTCGAGTTGTTTGGCCATAGGCTACCT |
| | | AATGATAAGTTATTAGCTAAGAAAGAATGGAATGAAATAGTGGAAGGTTCGCATCCCATA |
| | | TGGAAAAATATTTCGATAGAAGTTAAGGAAATGTGTCGTTCTTTTATTATCCATATGCAA |
| | | GCAGAACTCTTGAAAAAAATCAAACACTCGAATCCATTTCAGTTTGAGAGTGCATCCATT |
| | | GGAAATTTTTTCTTAACTGGTGCTAGATTGTTTTTAGGTTCTCTAGACGCATCTATTGAA |
| | | CTAATGATGAGGATAGGAAGGTGCAGTCCTCTAGTTCATGTTATTCCATGTATAAATACC |
| | | AATCACACACATCATATCTCGGCTTTGTTGACGAATGGTGAGATGATAACTGGCCAATCA |
| | | CAAATATCGCATCCCTCGAAATCTGTTCCTAAAGATAATAGCATAGCACATTCAGCTAAA |
| | | TTTATTCATTTACTTGGCTCTTATGACGACCATTTGAAAATTCTCCTCGACGATGAGGAA |
| | | GAAGAAGCGGAAGAGGAGTATGCCAATCCTATCTATATTCTTCCAGAACTGAAAAACTCC |
| | | CAGTTGCATTTTGACAAGCTTGATGAATCACAGAATCTACCTGCTCCGGTACATAGGATA |
| | | TTGTATATAAACCCATATGGCGAAGAGATAAAACCAATGGGAAATCCAAGAGCTATTTCC |
| | | AAGGTGAAAAAAGCTGACATGGTCGTTTATTCTATCGGTTCTTTAATGACAAGTTTACTC |
| | | CCAATCCTTATTCTTGGTAATCTTGCTGAAGTAATTTTGGAATCTAATAATACGAAAAAA |
| | | GTTTTGCTCATAAATAATAAATATGATAGAGAGGTTTTTGGTTTAGACGGTTTACACTAC |
| | | GTTCAAATGATCATAGATTCTATGAGTAGGGCAATAGCAGGCTATAGGCAATCTAAGGGT |
| | | GTCCATTCAGAGAATGATGACTTTGAATGGCAAGATTTTATTACTGATATTGTTTACCTA |
| | | AAAAATGGTGAAATTGAAATTGATGAAACGATATTCGAGAAGCATAGTATTAGATGCCAC |
| | | CAGATCGCGTCTTCGGATAAGATGGAAAGTGAAGAACTTGAAAAGGTTCTGAATCAAATT |
| | | GGCCTTAAAAACTGA |
| YLR262C-A* | TMA7 | ATGTCTAGTCGCCAAGGTGGTAAGATGAAGCCTCTAAAGCAAAAGAAGAAGCAACAACAG |
| | | GACTTAGATCCTGAAGACATTGCCTTCAAAGAGAAGCAAAAGGCCGATGCTGCTGCTAAG |
| | | AAAGCTTTGATGGCTAATATGAAAAGTGGTAAACCTTTAGTGGGTGGTGGTATCAAAAAA |
| | | TCTGGTAAGAAATAA |
| YKR010C* | TOF2 | ATGATAAAAATGTGGAGGTTACAGATTGTACTGGTGCCGCCAAGTGCTCAAGATATTATC |
| | | ACATTTCTCGAAGCACGCTTGAATACTCCACAATCAGTGTCTCCTATGGTCCAGTATAAT |
| | | GAAGATATAATTACGCACAACAACAGTATCAATAACTGTAGCGACCCCTCTCCTACGAGC |
| | | CCGTCTTCGCAAAATTCTATTCAGTCGAATCGTAGTAGTGATTTCATTAACTACCTCCCG |
| | | AATTGTAAAAAGTTTTTACATTTTACGGACGGTGACAATACCCTTTTACAACTATCTAAT |
| | | GAAATTTTAACTAAATTTGATAGACTTTATCCAAATTTCAAAGAATCCATTGAAATCGTA |
| | | TCTCTACAAGATAGGCATGGTTGTGATTTGGACTCTGAATTTATAATAAAAGACGTATTT |
| | | GAAAATGATGGTGTGGTTTTGGTAATTTTAAAAGATGAATTAGACTGGAGTAGAAATCAA |
| | | CATATTTCACTTTTACAATTGGCAAGACAGCGGAGACGGCAGGACAACAAGCCATCCACA |
| | | AAATCCATCGTTACTGAAAAGAGAAAAAAGATCTCTAAGGAAGACTTATCTTCTATATCA |
| | | AATAAAGATACAATGCATCTAATAGCTAAAAGCTCGCTCAAGAATAACTTCATTAATAAA |
| | | TCAAGAGTATCAACGCCGCTAATGAACGAAATTCTACCGCTTGCAAGCAAGTATGATGCA |
| | | CTGAATAAAGAAAAGTGTCCCATGCCACTTACTTCCACAGTAGTGGCATCAAATGTACAC |
| | | AAAGACGTAAAAGATCACGCTCGTGCTAAAGAGGGAGTTGTAACGCAAGGGAGTGATAAC |
| | | AATAAAGAAAATATACCTTCATCCACTCAACAACAGAAAAATGATGGCGCTAAAAGGGCC |
| | | GAATCAAAGGATTTAGATCTTTTACGCAATTCGTCAGAAGATGCTGATTATGAACCCGCA |
| | | GATGAAAACTCCCCTCAAATTTCGTTTGATTCGATTGATACCGACTTCCAATTGAGCACT |
| | | ACTTCTCATACTAATTCTGATATGCATATACAATATTCTAACCCCTCTAGTGGTGCTCAT |
| | | AGTCCCAGGAAGAGTTCCTTAGAAATTAAAGTTCAAAACAAAAAAGGTGATGATCTGCCA |
| | | TTGAACGACAAAGATATAGGTGAGAATTGTAGAAGGATAGAAGCATTTTCAGATGAAGAG |
| | | GATTTTAATGAAACCGATAATGATCGTGCGGATTCGTTTATCAACAATTCTAAGAAGGCA |
| | | TCGATGGGATTTCGGGATATCAATTCCGACTTGGATAGCGTTTCATTCAACAGTGATATT |
| | | GAAAATGCAGTACAAAGTACTCAAAGTACCAAAAATGTTGTTTCTCCTCCCTTTTTTCCT |
| | | GAAAAAGAATTGAATAATCGGTTACATCAAAGTCAAGGTAAGGAAGCCCTATTTCGACTT |
| | | GTTGAGAAGGAGTTTCCTGATAAATCATTGGGTGCTGCGTCATCTACTTCGCATGCAAAA |
| | | GACGTAAAAATACAAGAAACTATTCGGAAACTAAATAGGTTCAAGCCAACTGGGGAAACT |
| | | AAGGTCCAAAAAAGGAATAGTATTACGGAACCATATTACGGAAAATTTGGAATAATGAAG |
| | | AAAGATAAACCCAAATCCATAACTTCAAAAGGGGTAAGTCTGGAAACAAAGCACTTTGAT |
| | | GATCCCAATACCATTATTTCCGGTGAGAAATTCGCAAAATTTGGTAAAATAAAAGTGAAA |
| | | CGAAAAACTGATGATGTTGGATCGAAAGTTATAGAGTTTAAAAGAAAAAGAAATATGGGG |
| | | AATAGATCCCTAAAAGATATTTTTGCCAATGCTGGGAAACCGCCAAATGCAGCTTCGACC |
| | | ATAAAAGTAGTCAAATTAATGAGAGATCCTGTTGATAATAGTAAAGACAAAGTGGAGGCA |
| | | ACTTCTAATAGTACGGCTCAGGAACAAGAACAAGTCTCACCCAAGTTGCCAGTTATGAAT |
| | | TCAACTCCTGGAAAAAGAAAAAATGGCCAAGCGATACCGTCCAGTTTAGAAAGAACACCG |
| | | CAGCTCAAGAAAGTCAAGGTTACCAGGTCGCATTCTTCACCTTCTTCTTCTTCTTCCATG |
| | | TCACTTGAATCTTCATTGGATAGCTCTTCCTCAGATGATTCGGATGATGATTCTGATTCG |
| | | AGGAACGTGCAAGTTAAAAAAATTAATTTCAAAACAAGTCATGGACCAGCTGGAAATTCT |
| | | AATGGAAAACCAATGCTGGATGTGGATGACAATGAAATAAATACAAAGAAGTACCAAACA |
| | | CCTAAATATGTTGAAAGTGATGAAGACGACCAGTAA |
| YDL056W | MBP1 | ATGTCTAACCAAATATACTCAGCGAGATATTCGGGGGTTGATGTTTATGAATTCATTCAT |
| | | TCTACAGGATCTATCATGAAAAGGAAAAAGGATGATTGGGTCAATGCTACACATATTTTA |
| | | AAGGCCGCCAATTTTGCCAAGGCTAAAAGAACAAGGATTCTAGAGAAGGAAGTACTTAAG |
| | | GAAACTCATGAAAAAGTTCAGGGTGGATTTGGTAAATATCAGGGTACATGGGTCCCACTG |
| | | AACATAGCGAAACAACTGGCAGAAAAATTTAGTGTCTACGATCAGCTGAAACCGTTGTTC |
| | | GACTTTACGCAAACAGATGGGTCTGCTTCTCCACCTCCTGCTCCAAAACATCACCATGCC |
| | | TCGAAGGTGGATAGGAAAAAGGCTATTAGAAGTGCAAGTACTTCCGCAATTATGGAAACA |
| | | AAAAGAAACAACAAGAAAGCCGAGGAAAATCAATTTCAAAGCAGCAAAATATTGGGAAAT |
| | | CCCACGGCTGCACCAAGGAAAAGAGGTAGACCGGTAGGATCTACGAGGGGAAGTAGGCGG |
| | | AAGTTAGGTGTCAATTTACAACGTTCTCAAAGTGATATGGGATTTCCTAGACCGGCGATA |
| | | CCGAATTCTTCAATATCGACAACGCAACTTCCCTCTATTAGATCCACCATGGGACCACAA |
| | | TCCCCTACATTGGGTATTCTGGAAGAAGAAAGGCACGATTCTCGACAGCAGCAGCCGCAA |
| | | CAAAATAATTCTGCACAGTTCAAAGAAATTGATCTTGAGGACGGCTTATCAAGCGATGTG |
| | | GAACCTTCACAACAATTACAACAAGTTTTTAATCAAAATACTGGATTTGTACCCCAACAA |
| | | CAATCTTCCTTGATACAGACACAGCAAACAGAATCAATGGCCACGTCCGTATCTTCCTCT |
| | | CCTTCATTACCTACGTCACCGGGCGATTTTGCCGATAGTAATCCATTTGAAGAGCGATTT |
| | | CCCGGTGGTGGAACATCTCCTATTATTTCCATGATCCCGCGTTATCCTGTAACTTCAAGG |
| | | CCTCAAACATCGGATATTAATGATAAAGTTAACAAATACCTTTCAAAATTGGTTGATTAT |
| | | TTTATTTCCAATGAAATGAAGTCAAATAAGTCCCTACCACAAGTGTTATTGCACCCACCT |
| | | CCACACAGCGCTCCCTATATAGATGCTCCAATCGATCCAGAATTACATACTGCCTTCCAT |
| | | TGGGCTTGTTCTATGGGTAATTTACCAATTGCTGAGGCGTTGTACGAAGCCGGAACAAGT |
| | | ATCAGATCGACAAATTCTCAAGGCCAAACTCCATTGATGAGAAGTTCCTTATTCCACAAT |
| | | TCATACACTAGAAGAACTTTCCCTAGAATTTTCCAGCTACTGCACGAGACCGTATTTGAT |
| | | ATCGATTCGCAATCACAAACAGTAATTCACCATATTGTGAAACGAAAATCAACAACACCT |
| | | TCTGCAGTTTATTATCTTGATGTTGTGCTATCTAAGATCAAGGATTTTTCCCCACAGTAT |
| | | AGAATTGAATTACTTTTAAACACACAAGACAAAAATGGCGATACCGCACTTCATATTGCT |
| | | TCTAAAAATGGAGATGTTGTTTTTTTTAATACACTGGTCAAAATGGGTGCATTAACTACT |
| | | ATTTCCAATAAGGAAGGATTAACCGCCAATGAAATAATGAATCAACAATATGAGCAAATG |
| | | ATGATACAAAATGGTACAAATCAACATGTCAATTCTTCAAACACGGACTTGAATATCCAC |
| | | GTTAATACAAACAACATTGAAACGAAAAATGATGTTAATTCAATGGTAATCATGTCGCCT |
| | | GTTTCTCCTTCGGATTACATAACCTATCCATCTCAAATTGCCACCAATATATCAAGAAAT |
| | | ATTCCAAATGTAGTGAATTCTATGAAGCAAATGGCTAGCATATACAACGATCTTCATGAA |
| | | CAGCATGACAACGAAATAAAAAGTTTGCAAAAAACTTTAAAAAGCATTTCTAAGACGAAA |
| | | ATACAGGTAAGCCTAAAAACTTTAGAGGTATTGAAAGAGAGCAGTAAAGATGAAAACGGC |
| | | GAAGCTCAGACTAATGATGACTTCGAAATTTTATCTCGTCTACAAGAACAAAATACTAAG |
| | | AAATTGAGAAAAAGGCTCATACGATACAAACGGTTGATAAAACAAAAGCTGGAATACAGG |
| | | CAAACGGTTTTATTGAACAAATTAATAGAAGATGAAACTCAGGCTACCACCAATAACACA |
| | | GTTGAGAAAGATAATAATACGCTGGAAAGGTTGGAATTGGCTCAAGAACTAACGATGTTG |
| | | CAATTACAAAGGAAAAACAAATTGAGTTCCTTGGTGAAGAAATTTGAAGACAATGCCAAG |
| | | ATTCATAAATATAGACGGATTATCAGGGAAGGTACGGAAATGAATATTGAAGAAGTAGAT |
| | | AGTTCGCTGGATGTAATACTACAGACATTGATAGCCAACAATAATAAAAATAAGGGCGCA |
| | | GAACAGATCATCACAATCTCAAACGCGAATAGTCATGCATAA |
| YKR012C* | — | ATGAACGTCGTTAGAGTCTGGCCATTTTCTACACGAACTATACCTTGTACCACTACAGTG |
| | | GCAGCTTGGTGGACATGTTGCGTGACAGTCACAGCAACTGGGGCAGATAAAGCGACAGAT |
| | | GCAGATGCGGCCAGTAGTGAGACTTTAGAAAATTTCATCTTTGTAAGTTTTATTAAAGGA |
| | | ATGGTATTGTTTTATGATGGGCTGTTTCGAGGAACGATGATACCAAGAGCCAACAGATCC |
| | | ATCACAGCAATATGTAACACTCTGCGACAAGCATATATATATACAAAGATCCAAAGCATT |
| | | GCCTTTGAACGACGTCTATGTTTCCAAAACATTACATATAGAGAAGTGCGTGAGATACAC |
| | | GAAATGTACTGTAAATAG |
| YDL045W-A | MRP10 | ATGTCCGGAAAACCACCAGTTTATAGATTACCCCCTCTTCCCAGACTAAAAGTGAAGAAA |
| | | CCTATCATTAGGCAAGAAGCGAACAAATGTCTTGTTTTAATGTCAAACTTATTACAATGC |
| | | TGGTCTTCATATGGTCATATGAGCCCTAAATGTGCTGGCTTAGTAACGGAATTGAAAAGT |
| | | TGCACTAGTGAAAGTGCCCTCGGCAAAAGAAACAACGTTCAAAAGAGCAATATTAACTAT |
| | | CACGCCGCTAGGCTGTATGATAGGATCAACGGTAAACCCCATGATTAA |
| YDR533C* | HSP31 | ATGGCCCCAAAAAAAGTTTTACTCGCTCTTACCTCATATAACGATGTATTCTACAGTGAC |
| | | GGCGCCAAGACCGGTGTTTTTGTTGTAGAAGCCTTACATCCCTTCAACACATTCCGAAAA |
| | | GAAGGTTTTGAAGTCGATTTTGTATCTGAAACCGGAAAATTTGGTTGGGATGAGCATTCC |
| | | TTAGCCAAAGATTTTCTAAATGGTCAAGACGAAACGGATTTTAAAAATAAAGACTCAGAT |
| | | TTCAACAAGACATTGGCTAAAATTAAGACACCAAAAGAGGTGAATGCCGATGATTACCAA |
| | | ATTTTTTTTGCATCTGCAGGCCACGGTACCTTATTTGACTATCCTAAGGCTAAAGACTTG |
| | | CAGGACATTGCTTCCGAAATTTATGCTAACGGTGGTGTTGTCGCAGCTGTTTGTCACGGT |
| | | CCTGCTATTTTTGATGGGTTAACAGACAAAAAAACAGGAAGACCATTGATCGAAGGTAAA |
| | | TCTATCACCGGTTTTACTGATGTTGGTGAAACCATTTTGGGTGTTGATAGTATTTTGAAA |
| | | GCCAAGAATTTGGCAACCGTTGAAGATGTTGCTAAAAAATATGGCGCTAAGTATTTAGCT |
| | | CCGGTTGGGCCCTGGGATGATTACTCTATTACTGACGGAAGACTGGTAACAGGTGTGAAT |
| | | CCTGCTTCTGCGCACTCCACTGCCGTAAGATCCATCGACGCTTTAAAAAACTGA |
| YPR047W | MSF1 | ATGTTTCTCAATAGAATGATGAAGACCAGGACTGGTCTTTATCGCTTATATTCAACCCTT |
| | | AAAGTTCCACATGTAGAAATCAATGGCATAAAATACAAGACCGACCCACAGACTACCAAT |
| | | GTTACAGATTCAATAATAAAGCTTACCGACAGATCATTACATTTGAAAGAATCACATCCA |
| | | GTAGGCATTCTTCGCGATCTAATTGAAAAGAAATTAAACTCAGTCGACAACACATTTAAG |
| | | ATCTTTAATAATTTCAAGCCCGTGGTAACCACAATGGAAAACTTCGATTCTTTAGGGTTT |
| | | CCTAAGGATCATCCTGGAAGATCAAAATCTGACACATATTATATAAATGAGACGCACCTA |
| | | CTGAGAACACATACTTCAGCCCACGAATTAGAGTGCTTTCAAAAAATAAGAAACGATTCA |
| | | GATAATATTAAAAGTGGATTTTTAATATCTGCAGATGTGTACAGAAGAGATGAAATTGAC |
| | | AAAACTCACTATCCGGTATTCCACCAAATGGAAGGAGCCACAATTTGGAAACGAACGAAG |
| | | GCTGATGTGGGCGTAAAGGAGCCAATGTATATCGAGAAAATCCGTGAAGATATCAGACAG |
| | | GTAGAGAACCTTTTAAATAAAGAAAATGTAAAGATTACGGTTGACGATGATACTATACCT |
| | | TTGAAAGAAAATAATCCTAAACAAGAGTATATGTCCGATCTGGAGGTTGATTTGTGCTCT |
| | | CAACATTTGAAGAGGTCCATTGAACTGATAGTTTCTGAAGTTTTTAACAAAAAAATATCT |
| | | AGCATGATCAAGAACAAAGCGAATAATACACCCAAAGAGCTAAAAGTCCGTTGGATTAAC |
| | | GCTTACTTCCCCTGGACCGCGCCCTCATGGGAAATAGAGGTTTGGTGGCAGGGCGAATGG |
| | | CTCGAACTCTGCGGATGCGGATTGATTCGTCAAGATGTGCTACTAAGAGCCGGATATAAA |
| | | CCTTCTGAAACAATTGGGTGGGCTTTTGGCTTGGGTTTGGACCGCATTGCTATGCTTCTT |
| | | TTTGAAATTCCAGATATTAGACTGCTTTGGAGTCGTGATGAAAGATTTTCAAGACAATTC |
| | | TCCAAGGGATTAATTACTTCCTTCAAACCTTATTCAAAACACCCGGGATCATTTAGGGAT |
| | | GTTGCGTTTTGGTTACCAGAAGATAAACCAGATATTCATCAAGTTCATGAAAATGATTTG |
| | | ATGGAAATTATCAGAAATATAGCTGGCGATTTGGTAGAGAGTGTCAAGCTAGTCGATAGC |
| | | TTTACGCATCCGAAAACTGGGAGAAAATCTATGTGCTACAGGATCAACTATCAATCAATG |
| | | GACAGAAATTTGACAAACGCCGAAGTTAACACTTTGCAAGACATGGTGTGTTCTAAATTG |
| | | GTAAAAGAATACAGCGTAGAACTCAGATAG |
| YDL044C* | MTF2 | ATGATCAGAACATCATCTATATTAAAAAACTGTAACTATAGATACATACATTGCATCCAT |
| | | AGATGCTTGCTGAATGAAGCCAATTTGAAAGATAGAAAAACTCACAACGTAGAAAGAGTT |
| | | TCAAATGAAAAGACATTTGAACAAGCTCTCGAAGAGGAACGTAAGGTATTTGGAGAACTA |
| | | TTCGAAGCTGGAGCAAGGGTTGAAAACATGAGGCACACTAATGCTAGCAAAATAATTGAC |
| | | AAGTATTACAATGGGCTACAGGACAACTCCGAGGGAACTAGTGTAAAAAAAGAAAAAATT |
| | | GTTTTTAACCACAGTCAAAGAGCTCAAAGGAAGCTTCCAAATAAGGACCATGAATTCCTG |
| | | AAGGAAACAGCAGGCAATGACTATGTCTACGAACGGGCAGAACCTTCTGCTATTAGCACT |
| | | AAAACGATAAGTGAGCAGACAAGAACTTTGCTGGAAAAGATATTTGACGAAGATAATAGC |
| | | ATTAATAAAAGCAACAGAGAATTGCTGAATCTTAATCTGCGCAAGGGCTCGGGAATGGAA |
| | | GCTTTGAGGCAGCCAGTTGCACATTCCAACGTCAAATTTAGCGAGGAAGTGATGCAAGAA |
| | | ATTGGTAATAAGATTCGGTACCAAACCACACTGGATCAGGTTTTAGAACCTCACATTGAC |
| | | TACTTGAGGGAAGCTGTTAAAAGTGATTACGATCTTTTAAGATACCTAAAGCAATCACTA |
| | | GACATTTACAAGAAAAGGAACAAAGACCTAGAGTTAAAGATGAATGCAGAATCTTCAAAC |
| | | ATTTTCGAAGACATTAGAAGTGCGTGTATAAATAAACCGGCAGAACTACCGAAACCGCTT |
| | | GCAATGACTTTACCATATATTATAGTCAAATCGCTTAGATTAGGTGACTTTGATTTCCCG |
| | | GCAGACAGAAAGTACACATTGATCTCCTATGTTTATAATGAATGTAAAAACAACATGGAC |
| | | GCTTCACTGTACCTAACAATTTGTAATGTTGATTTCTACAATTTGCTGGTTCAGCTATTG |
| | | TGGGAAAACTTCCAGGAGATTCGCTACTTGAGACGTGTGGTTACCGAGATGAGCGTTAAT |
| | | GGTGTCATCGGAAATATTGAAACCGTGGATATATTGGATAAGATTGTCAAAGAAATGAGA |
| | | AGTTTAAATGAGGATGTCTTTCTTGAAGCAGGAGAACAGCTCAGTGCAGATGAGGAAGTT |
| | | TCGTCCAGCGCAAATAAAATAGTGAATGTAGGTGTTTTATGGAATAAAGACACGAATAAT |
| | | GACTTACTAATTGTTGAAAATTATCTCAAAAGCCTCAAAAAAAATTTAACTAGAGACAGG |
| | | TAG |
| YMR310C* | — | ATGAGCAGCACGAGGAAGTTCAAAAAGGTTGAGAAACCTCTATCACAAACCAGACATTAT |
| | | TCACTCTGTATCCCCACGACATTGGTATCGGACTGTCGCAACCTCTCTCAAATCACGCAT |
| | | AAGGTGTACCAAGTGGCCAAGTTTGCATCGCTGTTTAATGTTTCTGAGGTTGTCATTCTT |
| | | GAGGACAATTCCCAGGTCGATGCCACCAAAAAAAAAATCTCCACTGCTAAATTAATACTG |
| | | GCACTTTTACAATACTTCGTCACGCCTCCATATCTTCGCAATACTGTCTTCAACGAAAAA |
| | | TTCAGACCTTATTTGACCGCCGCTTCAAAATTACCAAGATTATCCACACTTCCTTTCACC |
| | | AGGTACCAAAAGCAAGATCACGGTCGGTACAGAGAAGGCCTCACTATCAAGATGCAGAAA |
| | | CCCACCTTGGCTCGCAAAAAGATTGGTAAGGTATTCAAGCAAACCAAATATATTAATATA |
| | | GGGAAAAGCAAAGCACTGGCTCTACAAAGTCAGCTGGTGCCGATAAACGCAAGGGTCACC |
| | | ATCGACACAATAACGAGGAAAATTGTATCTCCTCAGGAAGCTTACGGTGATTTCACCGGA |
| | | TTGGACTCGCAGTATGGCTACTATACTCGTATAGCGTCATCGTTCACAGACTTGTTTATG |
| | | AAGGGGCCTTTAAAAGAGGGTTATACACAATCAGTATATGTTCCCTTAACCACGAGAGAC |
| | | ACCTCTATTCCCGAGTTATCATCACTGCCTACCGCCGAAACTAATCCCCACATCTTATTG |
| | | GTATTCTCAACGTGGGACACTCTTGCTCGAGCATTCAAACTGGATCAAGATCAATTCGTA |
| | | GATTGTCAAGGTCCTCAAGAGTTCTTTGATGCTCAGCTACCATGCCCCGTATCCAATTCG |
| | | GACGTTGCAGATGCAATCCCCATGACACTGACAACTCTTTCAACTGTATTTTAA |
| YPR100W | MRPL51 | ATGGTTGTGAAAGCTATTGCACGAAATTCCATAGGTCGTAATGGAGTTGGAGCATTCGTG |
| | | TTTCCATGCAGAAAGATTACTTTGCAATTCTGTAACTGGGGAGGGTCTTCGGAAGGCATG |
| | | AGGAAATTTTTGACTTCAAAGAGATTAGATAAATGGGGCCAGGAGTTCCCATGGATACAG |
| | | TTTGAAGTGATGAGGAAGTCTGGACATCCTTTGTTGAGGGCAGAATACACAAATGGGCGT |
| | | GAAAAAGTTATTTGTGTGAGAAACTTGAACATTGACAATGTAGAAAACAAGCTAAAGCTA |
| | | TTGAAGGATTCTGATGGAGACATTTTACGCAGAAGAACAAAGAATGATAACGTGGAAAGC |
| | | TTGAACAGTAGTGTGAGGGGTATATGGTCGCCTCTGCATGCTGCGAAAAGGCATAGAATT |
| | | TAG |
| YHR038W | RRF1 | ATGATTTTAACCACAGCTAGATTAAATTGTAGACCAGTCACCGTTCCTCGTCTATTTAAT |
| | | CGTTCTTTTAGTCAATCTTTCATAATTTTGAAGAAAAAAAGTTCTACCCCTACTGAGAAA |
| | | GTCGAAGAAGATGAAATTGACGTGAATGAACTGCTGAAAAAGGCAGAAACTCAATTCAAA |
| | | AAAACTTTAGAAATTCAAAAACAGAAAATGAATGAGATAAAACAGGGAAATTTTAATCCT |
| | | AAGGTATTCAATAGTTTAGTGTTCAAAAATAACAGAAAGTTTACAGATATTGCTACCACA |
| | | TCCTTGAAAGGTAAAAATGCACTTTTAATAACAGTTTTCGACCCCAAAGATGTGAAAACT |
| | | GTGATCAGTGGGGTGCTTGCTGCGAACCTGAATTTAACTCCTGAAAGGGTCCCAAATAAC |
| | | GATTTGCAATTGAAAGTTTCGTTACCACCACCAACTACAGAATCCCGGTTAAAAGTAGCT |
| | | AAAGACTTAAAGAGAGTATTTGAAGAATATAAGCAGTCATCGCTAAAAGACTCATTAGGA |
| | | ACTATCAGAGGCAGTATTCTAAAGGAATTCAAAAGTTTCAAAAAGGATGATGCCGTTCGA |
| | | AAAGCTGAGAGGGATTTGGAAAAACTGCATAAGGATTACGTGAACAAGCTTCATGACCAA |
| | | TTCCAGAAAGTTGAAAAAAGCATTGTAAAATGA |
| YDR519W | FPR2 | ATGATGTTTAATATTTACCTTTTCGTCACTTTTTTTTCCACCATTCTTGCAGGTTCCCTG |
| | | TCAGATTTGGAAATCGGTATTATCAAGAGAATACCGGTAGAAGATTGCTTAATTAAGGCA |
| | | ATGCCAGGTGATAAAGTTAAGGTTCATTATACAGGATCTTTATTAGAATCGGGAACTGTA |
| | | TTTGACTCAAGTTATTCAAGAGGCTCTCCTATCGCTTTTGAACTTGGCGTTGGCAGAGTA |
| | | ATTAAAGGTTGGGATCAAGGTGTTGCCGGCATGTGCGTTGGCGAAAAAAGAAAGCTGCAA |
| | | ATTCCAAGTTCTTTGGCCTACGGAGAAAGAGGTGTCCCAGGCGTCATTCCTCCAAGTGCT |
| | | GATTTGGTGTTTGATGTCGAATTGGTAGACGTGAAATCAGCCGCCTAG |
| YJL119C* | — | ATGAACCACTTGACAACGGTCTTGGCCGCACGTAAAACCTGTGTGGTGCGAGAAGCAACG |
| | | AATCTGTATACAGTTGATACAGAACGTCGGATCAACAAAATGGGGCGTGATGAAGAAAAA |
| | | AAGTGGCGATCCCGTGGAGGACATTGGTGCATGGATGATTCAATCATACAATTTACGACA |
| | | TTTAACCAAAGTTGCACTCTGATGGGCTCAACGACTGCTACTCGTGCCAATAATGAAACT |
| | | GAAAAGCATGAAGCCATCACTGTTTTTTTCGTGGTCTTTCTATATATAAACAAAGCCTCT |
| | | GTAAAGTTTTTTTTTTTCGCATAA |
| YOL095C* | HMI1 | ATGGACAAGCTAACTCCATCTCAATGGAAGGTAATAAATAAGTCATATGAACCAGCGTCT |
| | | ACGATAAAAGTCATTGCGGGCCCAGGCTCAGGAAAAACGCTAACGCTACTATATAAAGTA |
| | | CTACATTTAATTACTGTGGAGAATATTAAGCCTGAAGAAATACTTATATTTTCGTTAACA |
| | | AACAAAGCAGTTGATAGTATAATAGAAAATTTACTGTCCATTTTCGAAAACTCGCATACA |
| | | AATAAGGAAATTGTCCATCAGATTGGGTGTTACACAGTTCATGGGTTGGCTAATAGAATC |
| | | GTTGTAGAGAATGAGGGGATGATAAACATTATAGAAGAGATTGGATGGAGAGGATTAATG |
| | | AAGTTGTTACCGCCGTCAAAGAGAACCCCCCATCATTTCAGATCATATAAGGAACTCGAG |
| | | AAAGTTGTTAAGGATTACAAGCTAAACAACGCAAAAAATAATAACCCCGTCATTGAGAAA |
| | | CTAGTTGAACTGATGGATAACTGTAAAGTGATGACCAATGATGATTTGATCATTCGCGCA |
| | | AAAAAATACTTAGAGCTTGATTCTTCTGATTCAGATGCATCGTCTTTTACTCAAGATCTC |
| | | CGCAATAAATACAAGGTTGTTCTTATCGATGAATTTCAGGACCTCTATCCAAGTTTAGCA |
| | | CCCTTAATTACCATGATTTGCAAAGGCAAGCAGCTCATTATGTTTGGTGATACAAATCAA |
| | | AGCATATACGGATTTTTGGGGAGTAACAACGAGATTATGTCACAATTAGACAATCTTCAC |
| | | CCAAAAAATTCCACGACTGTCCTGAAATTATTTGATAATTTTAGGTCTACCCCCGAAATA |
| | | ATCTCTCTAGCATCAAAAATTATCAATCGTCCACTAGCAGAGAAACAGATTATCGATGAT |
| | | ACTGATGAAACACCATCAGAGCTAGTTCGCAAGCTACCGAGTGGTGTTTCACCTCAAATA |
| | | ATGACATTTGATGATCTAGCCGCAGAATCTGAATTTATTATTGATAAAATTACCCAATTG |
| | | ATTTGTTCTTCTGCAAAGTTTTCCGATATAGCAATTCTATCAAGAACAAATTCACATCTA |
| | | ACGGCTATAGCCAGTATTTTGAAGAAATATGGTATTCCTTATCAAAAACTAAAGAGTCAA |
| | | CCTGATTGGATGGACGATTTACGAATTCAATTCCTTTTAGATATTCTAAAAGTGTGTTCT |
| | | TTAGCCTCCGACGAAAAGCATAATCGTGAGTTCAATACGGGTGATAAATGGCAAAGTAAC |
| | | TTCAGCATTTTGGTCACAATGAGCGCTCTCAAAGGGATTGGCGATGCTTCAATACAAGCC |
| | | TTGTATAAAGCATGCAGCCTGAAAAATCTGTCAATTTGGAAATACCTAACTATGGTTCCC |
| | | AATTTTGAATGGCCATTGGGCCTCTCCATAAAGAAAAAAATGGAAAATTACACTTCAAAT |
| | | TTATATGAAATGATCGAAAATGATCAAGTTCATCAATTAGATGATCCGATGGAACTTTTA |
| | | GAAAAAGTTGCTAGCATCACAAATAACTTAAACCTAAATCCTACCTACTTCCAATCCTTA |
| | | AGTGATGCTCAATCATCTTTAGAGTTTAAAACACATCTACAAGAGATGGCACAAGTAATG |
| | | AAGGTCTCAAAATCTAATAAGCCCCCGGGTATTTCATTTGTTAAATGGTTTCTAGAGACA |
| | | TATTTTGATCAAACGATGGTCTTTCACCAGTCTCAGCAGGCATTACAAACTACCGGTCCG |
| | | GGGACAGTAAAACTATCTACCATCCATTCTGCAAAGGGTTTAGAATTTCCCATAGTATTT |
| | | CTAACAAACGGGAGCATGTCCAATTTCCCAATGGATACAAACGCCCTTTATGTGGGGATA |
| | | ACTAGGGCAAGAAACTTGTTGTACATGTGCAACATGAAGCACGAGAGACTGGTCAGTAAA |
| | | TCATCACCATACTCAAGAAACATAATGTCTAATAATTTATTCTGGACTTACTACAATAAG |
| | | GATTTGAAACGATCTGTATGTGACGTTAAGGTGACACATGGTTATAATGTGCAGCGATAC |
| | | AATCAGTTACGTAAAAATTTTGGATTTTATAGAGCATATTCTTCACTAAGAGGTTGTAAG |
| | | AGCGTTTTCAGACGTATATAG |
| YGL064C* | MRH4 | ATGTCGCTTTTTTTCAAGCCAGTAATTTCACCACAGTGGTCTTTCCCCGTGCTTCTCAAG |
| | | ATAGGAGTGCGTTCTTATGCGGGAGGCCCAAGAACAAAGCATAAAGGAAACTCTCCATTA |
| | | GCTTCAGTGCCTACAGGCAGTAGTAATAAAAACCGAAAACAGAAGGCCAAAGGCAAAAAA |
| | | GGTAACAAAAAGAATGACCCTGACCAAGCATTCAACTTTGGAGAGTATGGTGGATTGAAA |
| | | AAAGACGTCGAGATGAATATGGACAGTACGAATAAACTCATTCAAAAAATATCGAATTTC |
| | | GACCAATTGCTTATACTTCCACCTGTCAGAGACGCTGTGAAAGAAATCATTTCCAAAGAG |
| | | TCCCTGAAGCTACAAGATAGTAGGAAAAAAACCAGTGAAAATATAATACCGAGTCCCATT |
| | | CAAACAGTGGCCATTAAGAGAATATCCAAAAATTTAATGGATCCAAAGTTACAAATACAT |
| | | GCGATTGCCGCTGAGACAGGTTCAGGTAAGACCATGGCCTACTTGATACCTTTAATAGAC |
| | | TATTTGAAAAGACAGGAGTTGGAAACTCCCGAATTATGGGAAACATTAAGAAAAAACGTT |
| | | CTGATTCGGTCCATCATACTGGTGCCTACGCATGAATTGGTTGACCAAGTATACGAAACT |
| | | GTTTCCAAGACGAAAACCTTGTTAGGCTTAAACTCTTTCAAGTGGGACAAGGCCACATCA |
| | | TATCGTGATTTATTAGAGAACATTAAAAATAGAATAGATATTTTAGTCACTACTCCGGGA |
| | | AAACTACTGAACCTGTTCTCCATAAGAATGATAACTAGACCTGATAAAGTCTTGTCCAAA |
| | | GTGGGTTTCGTAGTACTAGATGAAGCTGACACACTTCTAGATAGGTCATGGCTTGAAGAA |
| | | ACGCACTCCGCAATAAAGAGAATTCCTAACATTAATCACCTTATTTTCTGTTCCGCTACT |
| | | ATTCCTCAAGAGTTTAATAAAACAATGCAAAGACTATTTCCTACAGTGGTTCCTATAATG |
| | | ACACCCAGACTGCATAAGTTACCATTCGCTTTGGACTTTAAAGTTATAAATTCTGCCTTA |
| | | AGTCCTTTCAAGGGATCCAAGATCAAAGCATTGGCACAAACTTTATATGCTATTTCAAAT |
| | | GATGACACGGAACCAGGGTTCGAGAAAAGATGCATTATCTTTGTTAACGAAAAAAAGAAT |
| | | GTTCCTGAAATTGTTAACCTATTGAATAAGAAATTCGGTCATAATGCCATCGGTTTAACT |
| | | GGTGAAGATACATTTGAAGAAAGATCCGAGAAAATTATGCCCTTTTTATCACCACCGCGA |
| | | CCACTTTCGGAGGTGGTTGCTCAGAGTACTTCACCTCCCACTTCCTTGAAGAAATTTGAA |
| | | ATTCCTGATTCCAACATCGTCATCGGCAAATTGAAAAACACAAATTCTAATGGTACCGCA |
| | | CCCAGTAACAAGTCTTTGCATGTACTCGTAACCACCGACCTCATGGCAAGAGGGCTCAAT |
| | | TTCAAAGGTGTACGGAATGTTGTTCTCTACGATGTACCCAAAACGTCAATAGACCTGATA |
| | | CACCGAGTGGGCAGAACTGCTAGAATGAAACAGGGCGGCCGCGTATTTATGCTAACTGAC |
| | | AGTAAAACAAAATCTTGGGCTAAAGCTCTTCCAAAGATCATCAAAAAGCACCAGAGATTA |
| | | TCATGA |
| YFR019W | FAB1 | ATGTCATCGGAAGAACCCCATGCATCTATATCCTTTCCAGATGGCTCACATGTCCGGTCG |
| | | TCCTCCACTGGTACTTCATCTGTGAACACAATCGATGCTACCTTATCCAGACCGAACTAT |
| | | ATTAAGAAACCGTCCCTGCACATTATGTCGACATCTACGACGTCGACTACGACGGATCTA |
| | | GTGACAAACCCAATTCTGAGCAATATCTCCGTGCCAAAAATAAGCCCCCCCACATCATCC |
| | | TCGATAGCAACAGCGACATCAACAAGTCACGTGACCGGCACTGCGTCGCATTCGAACATT |
| | | AAAGCTAATGCTAACACTAGTACCAGCGTGAATAAGAAAAATTTGCCGCCAACAACTAGT |
| | | GGGAGGATACCCTCTAGCACCATCAAGAGATATCCGAGTCGCTATAAGCCTTCTCACTCG |
| | | CTGCAGCTGCCCATTAAGAACGACTCCAACTTTAAACGATCTAGCATATATGCCTCCAAA |
| | | TCCACTGTTACTGCTATCCCCATTAGGAACAATCGGCCAATTTCCATGCAGAATTCCTAT |
| | | GCGCGCACTCCTGACAGCGATCATGACGACGTTGGCGATGAAGTGTCTTCGATAAAGAGT |
| | | GCCTCTTCTTCGTTGACCGCATCCCTGTCCAAGAGTTTCCTGTTCGCATTTTATAACAAC |
| | | CGCAAGAAGGACAAGACCAGTAACAATGGTGTCCTCTCAAAGGAATATTGGATGAAGGAC |
| | | GAGAGTTCTAAAGAATGTTTCAGTTGTGGTAAAACGTTTAACACTTTCAGAAGGAAACAC |
| | | CACTGCAGGATATGCGGCCAAATTTTTTGCAGCAGTTGCACTCTCTTGATAGATGGTGAT |
| | | CGATTCGGCTGTCATGCCAAGATGCGAGTATGTTACAATTGTTATGAGCATGCAGACACA |
| | | TACGAGGACTCCAGTGATGAAGAGAATGATTCTACAATGCAATTGAATGAGCCAAGGTCT |
| | | CGCTCTCGCTCTCGTTCTTCCAATACCAATCCATATTCTCATTCACATTCGCATTTGCAT |
| | | CTCATTTCACAGGACAACCATAACGGTACCGATCTCCATGATCCAGTAGCAGCAACGGAT |
| | | AACCCTCAACAGCAGAATGAGGTCTACCTATTAAATGACGATGACGTACAAAGTATAATG |
| | | ACCTCAGGTGAAGATTCTAAATTGTTCATTTCTACACCCCCACCACCACCGAAGATGGCC |
| | | ATCCCAGCAACAAAGCAAGGCGGATCGCTGGAAATCTCATTCGATTCGGAAAACGATAGG |
| | | GCTTTGCATTATCAAGATGACAACCCCGGTCGCCATCATCACCTCGATTCCGTGCCCACA |
| | | AGGTACACTATCAGAGATATGGACAATATATCACATTACGATACCAACTCTAATTCCACT |
| | | TTGAGGCCACATTATAACACCAATAACAGCACTATTACCATCAACAACCTCAACAACACT |
| | | ACTTCCAACAACAGCAACTATAACAATACCAATAGCAACAGCAACATCAATAATCCTGCG |
| | | CATTCCTTAAGAAGGTCTATTTTCCATTATGTAAGCAGCAATAGTGTTAACAAGGACAGT |
| | | AACAATAGCTCTGCAACACCCGCTTCGTCTGCACAGTCTTCTTCCATACTGGATCCTGCC |
| | | AATCGTATTATCGGGAATTATGCTCATAGAAATTACAAGTTTAAATTCAACTACAATTCC |
| | | AAAGGTCCTTCGCAACAAAATGACACGGCAAATGGAAATAATGATAATAATAATAATAAT |
| | | AATAATAATAATAATAATAATAATAATAACAGTGCCAGCGGTATTGCTGACAACAATAAC |
| | | ATTCCCTCAAACGACAATGGCACGACGTTCACTCTAGATAAGAAGAAAAGAAACCCACTG |
| | | ACAAAATCTAAATCTACGTCCGCTTACCTAGAATATCCTCTAAACGAGGAAGACTCTTCT |
| | | GAGGACGAAGGTTCCATGTCCATATATTCTGTATTGAATGATGACCATAAAACTGACAAT |
| | | CCAATTAGATCAATGAGGAACTCCACAAAATCTTTTCAACGAGCGCAAGCTTCACTACAG |
| | | CGGATGAGGTTTAGAAGGAAAAGTAAAAGCAAGCATTTTCCCAACAATTCGAAAAGTTCC |
| | | ATTTATAGAGACTTAAATTTCTTGACCAATAGTACTCCCAATTTACTTTCTGTAGTAAGC |
| | | GACGATAATCTATACGACGATTCTTCTCCTTTGCAAGACAAGGCCTCTTCATCCGCTGCA |
| | | AGCAGACTCACTGACAGAAAATTCAGCAATAGCAGTGGCAGCAATAATAATAGCAATAGT |
| | | AATAGCAATATCAACACCGATCCTTGGAAGAGAATCGCTAGTATATCGGGATTCAAACTG |
| | | AAAAAGGAAAAAAAGAGAGAATTAAATGAAGTCTCTCTTTTACATATGCATGCCCTATTA |
| | | AAACAATTATTGAATGATCAAGAAATTTCCAATCTTCAAGAATGGATAACCCTTCTAGAT |
| | | GGCGCTTTGCGTAAAGTTTTAAGGACCATCCTGAACGCGAGGGATTTGAACACATTAGAT |
| | | TTTAGACAAACTTACGTTAAGATAAAAAGAATTAGTGGTGGATCTCCTCAAAATTCAGAA |
| | | TATATCGACGGGGTTGTCTTTTCGAAAGCATTGCCGTCGAAAACAATGCCACGGCATTTG |
| | | AAAAACCCAAGGATACTATTAATCATGTTTCCACTGGAGTACCAAAAGAACAATAACCAC |
| | | TTTCTAAGTATAGAGTCCGTCTTTAGGCAAGAGCGCGAATATTTGGATAAACTTGTTTCG |
| | | AGATTAAAGTCATTACATCCTGATATTATATACGTCGGCGCCAATGTTAGTGGGTACGCG |
| | | TTAGAACTATTAAATGACTCAGGCATTGTCGTACAGTTTAATATGAAACCACAAGTCATT |
| | | GAAAGAATTGCCAAGCTAACAGAAGCAGATATTGCCATTTCTGTGGATAAGTTGGCTACG |
| | | AATATTAAGATGGGTGAATGTGAAACTTTTGAAGTTAAAAGCTACATTTATGGAAACATC |
| | | AGTAAAACATATACATTTCTTCGGGGCTGTAACCCAGAACTTGGTGGAACAATTTTGTTA |
| | | AGAGGTGATAGTCTTGAAAACTTGAGAAAAATCAAACAAGTCTCTGAATTTATGGTCTAC |
| | | GCAATTTTTTCATTGAAGTTGGAAAGTTCATTTTTTAACGATAATTTCATACAACTTTCT |
| | | ACAGATGTTTATTTGAAGAGAGCTGAATCCAAAAAACTGCAAGTTTTCGAAGGTTATTTT |
| | | GCAGATTTCTTGATCAAATTCAATAACAGGATTTTGACTGTATCACCAACGGTAGATTTC |
| | | CCCATTCCCTTTTTATTGGAAAAAGCCAGAGGCTTGGAAAAGAAGCTCATAGAAAGGATC |
| | | AATCAATATGAAAGCGAATCTGATTTAGATCGGCAAACGCAACTAAATATGCTTCAAGGC |
| | | TTAGAATCGACTATCACTAAGAAACACTTAGGAAATTTAATAAAATTCCTGCATGAAATG |
| | | GAAATTGAAAATTTAGAGCTTGAGTTTCAGAAGAGAAGTCGTCAATGGGAGGTTTCATAT |
| | | TCATCATCACAGAACTTACTTGGTACGGGCTCACACCAGTCTATAACAGTTCTCTACTCA |
| | | ATGGTTTCAACAAAGACTGCCACACCTTGCGTTGGCCCTCAAATTGTTACTATTGACTAT |
| | | TTTTGGGACAGTGATATTTCCATTGGTCAGTTTATCGAGAACGTCGTGGGAACTGCCAGG |
| | | TATCCCTGCCAACAAGGCTGTAATGGTCTGTATTTGGATCATTATAGAAGCTATGTTCAT |
| | | GGGTCAGGAAAAGTAGACGTTTTAATCGAAAAATTCCAAACTAGATTACCAAAATTAAAA |
| | | GATATTATCCTCACGTGGAGTTATTGTAAAAAATGTGGGACATCTACTCCGATTTTACAA |
| | | ATTAGCGAAAAAACCTGGAATCATTCTTTCGGTAAATATTTAGAAGTCATGTTTTGGAGT |
| | | TACAAAGATAGCGTGACAGGAATTGGGAAATGTCCTCATGATTTCACTAAAGATCATGTC |
| | | AAATACTTTGGTTACAATGATTTAGTGGTTCGACTAGAATATTCTGATTTAGAAGTTCAT |
| | | GAATTGATAACCCCACCACGCAAAATAAAATGGAAACCTCATATCGACATCAAACTGAAG |
| | | GTGGAACTATACTATAAGATTCTGGAAAAGATCAATAATTTCTATGGGAGTGTCCTAAGT |
| | | CGGTTGGAACGTATTAAATTGGATAGTATGACTAAAGATAAAGTTCTTAGTGGACAAGCT |
| | | AAAATTATCGAATTGAAGAGCAATGCTACAGAGGAGCAAAAATTAATGTTGCAAGACCTG |
| | | GATACTTTTTACGCAGATAGTCCTTGCGATCAACATTTACCATTGAACCTGGTTATCAAA |
| | | TCTCTATACGATAAGGCTGTAAATTGGAACTCTACATTTGCGATATTTGCAAAAAGCTAT |
| | | TTGCCTTCTGAAACTGATATTAGTCGTATCACGGCCAAGCAGCTGAAAAAACTGTTTTAC |
| | | GATTCTTCAAGAAAAGACAGCGAAGACAAAAAATCACTTCATGATGAAAAGGCGAAGACG |
| | | AGGAAACCAGAAAAGAACGAATTACCGTTAGAGGGATTGAAAGATGTCGAAAAGCCGAAA |
| | | ATTGATAGCAAGAATACGACAGAGAATCGTGACAGAACGAACGAACCACAGAATGCCGTG |
| | | ACGATAACCACTTTTAAAGACGACACTCCAATTATTCCAACAAGTGGTACTAGTCACTTG |
| | | ACAGTTACACCTTCTGCATCGTCGGTTTCTTCTTCTTTGACCCCTCAAACCGAAGAACGT |
| | | CCGCCAATAAGCAGAAGTGGAACAGGTATATCTATGACTCATGATAAAAGCACTAGACCT |
| | | AACATAAGAAAAATGAGTAGTGATAGTAGTTTATGTGGACTAGCATCACTTGCTAATGAA |
| | | TATAGTAAAAATAATAAGGTGAGTAAGCTAGCTACATTTTTCGATCAAATGCATTTTGAT |
| | | GCCCTGTCGAAAGAATTTGAACTGGAACGTGAAAGGGAAAGATTACAACTTAATAAGGAT |
| | | AAATACCAAGCCATACGATTACAAACATCCACACCAATTGTGGAAATTTATAAAAATGTC |
| | | AAAGACGCAGTTGATGAACCGCTTCACTCTAGAAGTTCAGGCAATAATTTGTCATCTGCG |
| | | AATGTTAAGACTTTGGAGGCACCTGTGGGAGAACATTCCCGGGCCAATAATTGTAATCCT |
| | | CCTAATTTAGATCAAAACTTGGAAACTGAATTAGAAAATTCCATTAGCCAATGGGGAGAA |
| | | AACATTTTGAACCCCTCTGGAAAGACCACCGCAAGTACTCATTTAAATTCTAAGCCAGTA |
| | | GTAAAGGAAACTTCAGAAAATCCAAAGTCAATAGTTAGAGAATCAGATAATTCAAAGTCG |
| | | GAGCCGCTTCCACCTGTAATAACAACAACAACTGTCAATAAGGTAGAATCCACTCCACAA |
| | | CCTGAAAAATCATTATTGATGAAAACCTTGAGCAACTTTTGGGCAGATCGATCTGCGTAT |
| | | TTGTGGAAACCCCTTGTTTATCCAACCTGTCCTTCGGAACACATTTTTACTGATAGTGAC |
| | | GTTATAATCCGCGAAGATGAACCAAGTTCCTTGATCGCATTTTGCTTAAGTACTTCTGAC |
| | | TATAGAAATAAAATGATGAACTTGAATGTCCAGCAACAACAGCAGCAGCAAACAGCAGAA |
| | | GCTGCTCCTGCCAAAACAGGAGGAAACAGTGGAGGAACTACGCAAACCGGGGATCCTTCT |
| | | GTTAACATTTCTCCCTCAGTTTCTACAACTTCTCATAATAAGGGGAGGGACTCAGAAATT |
| | | TCATCCTTAGTAACCACCAAAGAGGGCTTGTTGAACACCCCTCCCATTGAAGGCGCCCGA |
| | | GATAGGACCCCACAAGAATCACAAACGCATTCCCAAGCAAATTTAGATACTCTTCAAGAA |
| | | TTAGAGAAAATTATGACAAAAAAAACAGCCACACACTTGAGATATCAATTTGAGGAGGGT |
| | | TTAACGGTAATGTCATGTAAAATTTTCTTCACAGAACATTTTGACGTGTTTAGAAAGATT |
| | | TGTGATTGTCAAGAAAACTTCATTCAAAGTTTGTCAAGATGTGTTAAATGGGATTCCAAT |
| | | GGGGGGAAAAGTGGGAGCGGGTTTTTAAAGACTTTGGATGATAGATTTATTATAAAGGAG |
| | | CTGTCACACGCAGAACTGGAAGCCTTCATAAAATTTGCACCAAGTTATTTTGAATACATG |
| | | GCACAAGCAATGTTCCATGATTTGCCCACGACTTTGGCTAAAGTATTTGGGTTTTATCAA |
| | | ATTCAAGTAAAAAGCTCTATTTCAAGTTCCAAAAGTTATAAAATGGACGTTATCATTATG |
| | | GAGAACTTATTTTATGAGAAGAAAACAACAAGAATATTTGATTTGAAGGGTTCCATGAGA |
| | | AATAGACATGTCGAACAAACAGGTAAAGCTAATGAAGTGTTGTTGGATGAAAATATGGTA |
| | | GAGTATATTTACGAATCACCTATTCATGTTCGCGAATATGACAAGAAACTTTTAAGAGCT |
| | | TCTGTTTGGAACGACACATTATTTTTGGCCAAAATGAATGTGATGGATTATTCCTTAGTC |
| | | ATTGGTATTGACAATGAAGGGTATACTTTGACAGTAGGAATCATTGATTTCATTCGAACG |
| | | TTTACATGGGACAAAAAACTAGAAAGTTGGGTCAAAGAGAAAGGCCTAGTAGGGGGGGCC |
| | | AGTGTGATCAAGCAGCCTACCGTTGTTACACCAAGACAATATAAAAAACGTTTTAGGGAA |
| | | GCAATGGAGAGGTATATTTTGATGGTTCCTGATCCGTGGTATTGGGAAGGAAATTAA |
| YDR529C* | QCR7 | ATGCCACAGTCTTTTACGTCTATTGCGAGAATTGGTGACTATATTTTGAAGTCACCCGTC |
| | | CTCTCCAAGTTATGTGTTCCAGTTGCCAATCAGTTCATTAACCTCGCAGGTTACAAGAAG |
| | | TTAGGGCTCAAATTTGACGACTTAATTGCAGAGGAAAATCCCATCATGCAGACCGCTTTA |
| | | AGAAGACTCCCTGAAGATGAATCTTATGCCAGAGCATATAGAATAATCAGGGCTCATCAA |
| | | ACCGAGTTGACTCATCATTTACTGCCAAGAAACGAATGGATCAAAGCCCAAGAGGATGTT |
| | | CCTTACCTGTTGCCATACATATTAGAAGCTGAAGCTGCAGCTAAGGAGAAGGACGAGTTA |
| | | GACAACATAGAGGTCTCCAAATGA |
| YDR065W | — | ATGGCTCAAAATTTTGGAAAGATTCCTTCTCACAAGTCTTACGTATTGAGCTTATATCGG |
| | | ACCGTTCTCAGAAACATTCCAAAGTGTTGCCACTCATATGCGTTTCAATATGAAATAAAA |
| | | AAAACACTTTCAATACAATTGTTCAAACACAAGCATGACAAGTCAAGTTGGAGTGTTTAT |
| | | ACTTTACTCAATGAATTCAGTTTGTTGAACAATTGCCTTTTGGAAGGTAAATTACAAGAA |
| | | ATCAAAAACTTAATGAAGCCATTAAAAAAGATGAAAAAACAACTGAAAACAACGAAAATC |
| | | CTCAACTCTTTGACTTCATTAGGGGATGTAAAGACCAACGACCCAGAAGAAGTAAGAAGA |
| | | TTTCATGTTCTAAGTGCTTACATTAAGCGAAAACAAGATTTGGGCCTTTTACCTGCATAC |
| | | ATACCAAAAACGTATCAGCACAAACTCCTATTACCATTAGCATTGAACGAACATGCGTGC |
| | | CTGAAACTATTTCATATTCAGCAAAAATTGAAAAATGGCCCACCTTCTGCTGGGCTGTCA |
| | | TATACAAAAGAAGGAAGGAATCAAATATGGTTTGTTCGGTCACCAATTAACAAGGGCAGA |
| | | CAACAATCGAAAAAGCTGGGAATATTGATACGAAAAGAAAGGAAGGATTCTCAGAAAAAT |
| | | ATCGATAATCTCAATTTTTGCGAAATTAATGCTGCTTGGGCATTACATGAGGCCATTTGG |
| | | GAGGAATATCTAGAGTCGAAAAAGATTATTAAAGTAAACTTACCGAAATACTTGGAATAC |
| | | GCTGCAAATATCCCAAAGTCTACTAAATGTAATCCTTCGAGTCAATATCAGAAAGTTAAA |
| | | GAATGGGTAGATCCCGTGAGAGAAATAATGTTCGAATTACATTCCAAAAGTTTTCAAAGA |
| | | GTGGAGTACTTTAACAAATATAAGGAAAAGCTTTTAAAGAACGGTGGCCAACTAGCATAT |
| | | TTTGATAAAAAGTCAAAAGAAATGTACGCCAAACGGTTGACACTATTCAGAAAAATGAGC |
| | | AAAGAAACATTACCATATGTGACACTCTTTATAGAAGGGAGGGACTTGCCAAGTGTTTTG |
| | | GCGAAATACGGTTTTTAA |
| YGR283C* | — | ATGGCAGTAAAACATAAATCCGAGAGCCTTAAGCACGAAGAGGGGGCTGCCAAAAAGGCA |
| | | AAGACAGGCCTATTGAAGCTAAAAAAAATCATGGATATAGAATCTAATGTGGTCAAATAT |
| | | TCCATATGCATACCGACAACTGTTATCGACAATTGTAATAATTTGGAGCAAGTTACATTT |
| | | ACTGCATATCAAATAGCTCGGACAGCTGTATTGTTCAATGTTCAAGAAATCATAGTTCTA |
| | | GACCAGTCAAAAGATAAAAAGCATGAAAAAAAATCAAGATCAAAGGAGACTATCTCTGAT |
| | | TGTTTATTACTTGCCACACTATTACAATATTTTGTAACGCCTCCAAACTTACTGGATACT |
| | | ACATTCAAAAAAAAAAACAAGTTATATTTAAAGTGTGCATCTACCTTTCCGCCATTGAAC |
| | | CAGCTACCTTTTATGAACGCATCTGCCGAACAACACTACAAGGAAGGCTTATCAATCGCA |
| | | CGAGATAGCTCAAAAGGTAAATCTGATGATGCTCTAACAAATTTGGTGTATATAGGAAAG |
| | | AATCAAATTATTACGTTATCTAACCAAAATATTCCCAATACTGCGCGTGTAACAGTTGAT |
| | | ACAGAGCGCAAAGAAGTAGTGTCACCCATAGATGCTTACAAAGGAAAACCACTCGGCTAT |
| | | CATGTTCGAATGGCAAGCACTTTGAATGAGGTGTCCGAAGGTTACACAAAGATCGTTTGG |
| | | GTCAACAGTGGTGACTTCCACTACGACGAAGAGTTGTCAAAGTACCACAAGGTAGAAACA |
| | | AAGCTACCATACATCGCAAAACTGAAAAAAAGTTCAACATCAGAAAAACCCTGTAATATC |
| | | CTGCTTATCTTCGGGAAATGGGGCCATTTAAAACGTTGTTTCAGGAGATCTGACTTAGAA |
| | | TCCTCTTCTTTGCATCATTACTTTTCTGGTCAACTGCAATTCCCTGCTTCCGTACCCCAG |
| | | GGAAATATACCCATTCAAGATAGCCTACCCATAGCTCTTACCATGTTTCAGCGTTGGGCA |
| | | AGTTGA |
| YJL152W | — | ATGCCGCATTTAGCCGCCGAAGCGCATACTTGGCCTCCGCATATTTCACATTCAACACTT |
| | | TCGATTCCGCATCCAACCCCGGAACACCGGCACGTATTTCATAAAAAGGACGTGAAGAAC |
| | | AAAAGGAACGAAGAAAAAGGCAATAATTTACTCTATGTGTTATTTAGAACTACGGTGATA |
| | | AAGAGCTCGTTCCGATCACTAAGTACGGCCGGAAGAGAGCTGTTGTTTGTTGTCCATCAA |
| | | GGGCACATCGGCACCGGCCTCATCGTCTTCATCATATGCTGGAGGCTGTGCTTGAGATTC |
| | | CTCTGCAGGGTGAGCTTCCAGGTCACGGTCTACGGCGGGCGCAGTCGCATGTCTGCGTGA |
| YGL119W | ABC1 | ATGGTTACAAATATGGTGAAATTGAGGAATTTAAGGAGATTGTATTGTTCATCCCGTCTC |
| | | TTAAGAACTATACAGAACGGGAGAATTTCTTCTGTTTCTTCAATATCGTTGAGTAAGAAA |
| | | TATACTACAAAATCTGCCAAAGAAGGTGAAGAAAACGTAGAAAGGAAGCATGAGGAGGAA |
| | | AAAAAGGATACATTAAAAAGTTCCAGTGTACCAACTTCACGAATATCGAGATTGTTCCAT |
| | | TATGGCTCACTGGCAGCAGGGGTGGGCATGAATGCCGCAGCAAAAGGCATATCGGAAGTT |
| | | GCAAAGGGCAATTCTCCCACTTGGAAATCTTTGATTCTATCAGATTCGAATATTGACAGA |
| | | ATTACCAATAAATTTTCTAAAATGAGAGGTGTTGCCCTTAAAATAGGGCAACTATTGTCT |
| | | TTTCAGGATGAGAAAGTGTTGCCAAAGGAATTATACGAAATTTTATCAAGGGTACAGAAT |
| | | AGTGCAAATCATATGCCTCAAAGGCAATTGGAAAAGGTTATGGCCAAGGAATTAGGTGCT |
| | | AATTGGAAGACAAAGTTTTCCAAATTTGACAAGATACCTATGGCAGCTGCTAGTATCGGA |
| | | CAGGTACATGCGGCAGAGTTGCCCAGTGGTCAAAGAGTGGTTGTCAAAATTCAATATCCT |
| | | GGGGTTAAAGAATCCATTGATTCGGATTTGAATAGTTTACTAATGTTACTTACAGCCTCA |
| | | AGTTTATTACCGAAGGGATTATTTTTGGATAAGACAATTGCCAACGCTAGAACTGAGTTA |
| | | AAATGGGAATGTGATTACAACAGAGAGGCTAGAGCTCTACAAAAGTTTGAGGCGTTGCTA |
| | | AAGGATGACCCAGCGTTTGAGGTTCCTCATGTGTTTCCTGAATATACAACCGATAATATT |
| | | ATAACTATGACACGTATGGAGGGAACTGAAATAATGAAATTGCCCAAGGCATCACAAGAA |
| | | ACCAAAAATTTTATTTCGGAGAATATCATGAGACTATGTCTGGAAGAAATTGCCACTTTC |
| | | AAGTATATGCAAACGGATCCTAATTGGGCAAATTTCCTCTATAACGGTAGGACAAAGAAA |
| | | ATTGAATTATTGGATTTCGGTGCCTCCAGACCTTTTGCAGAAGATTTTATTCTAAAATAT |
| | | AGAAAACTTTTAACTTACGCCACATTGAGAGATAGGAAAGGGGCGTACGAAATGTCTGTA |
| | | CAACTGGGCTATCTCACAGGCTTAGAATCGCAGTCCATGAAAGACGCGCACGTCGATAGT |
| | | GTACTTACACTTGGTGAACCTTTTAGGGGTGATGTAGATAAATCTTTCGATTTCAAGGAT |
| | | CAAACAGTCTCAGACAGGATTAGAGGTAATATCGGGTTAATGTTGAATGAAAGACTATGT |
| | | CCGCCACCAGAGGAAACATACTCGTTACATAGAAAATTTAGTGGCATTTTTCTTTTGTGT |
| | | GCAAGAATGGGTGCGTCTGTTCATTGTGCAAAGCTTTTTAAAGAGATTTTTGCCTATAAA |
| | | GTTTAA |
| YHR051W | COX6 | ATGTTATCAAGGGCCATATTCAGAAATCCAGTTATAAATAGAACTTTATTGAGAGCCAGA |
| | | CCTGGTGCTTATCATGCAACTAGATTGACTAAAAATACGTTTATTCAAAGTAGGAAGTAT |
| | | TCTGACGCACATGATGAAGAAACCTTTGAGGAATTCACCGCAAGATACGAAAAGGAGTTT |
| | | GATGAAGCCTATGATTTGTTTGAAGTGCAAAGAGTGCTCAACAACTGTTTTTCCTATGAT |
| | | TTAGTTCCTGCTCCTGCTGTTATTGAAAAAGCTTTGAGGGCTGCCAGAAGAGTCAATGAC |
| | | TTACCTACCGCAATTAGAGTATTTGAGGCTTTGAAATACAAGGTGGAGAATGAAGACCAA |
| | | TACAAGGCCTACTTGGATGAATTGAAGGATGTCAGACAAGAATTGGGCGTTCCCTTAAAG |
| | | GAAGAGCTATTTCCAAGCTCTTCTTAA |
| YMR064W | AEP1 | ATGATTACTACAGTGCAAGAAATCAGCAAATGGAGAAACCTGTGCTTCATTAGGATGCAA |
| | | TCTCGCAAATGGTACCCAGTACTTAAAAAGACTCCTTTAGTAGCGGACGGTCGAAAGATC |
| | | ATTAAACATGCCGATAAAGTTCCTCATCCGGAAGAAATAATTCACCCATTTTACCAACCA |
| | | ACAGCAATAGAACAATTCACGGCATGCGCTACGGAATACAATCCTAGCCTGCTTGATGGC |
| | | AAAAAAATTGCTCCATCACTGATAAAACACCCGGTGAGCCTAAAAACCATACTAGTTGAT |
| | | AGCAAATTAAAGTTTGATGATATTAGGGGAGTGAATAAATGGTTGATGGAATTCGTTGCA |
| | | AGACGACAACATCAAAGAAATATTGTACTTACACCTGCGAGCAAAAGCGTTAGATCTTTT |
| | | CACGTTCTGCATTTGTCTTCAACCGATATTGCCAAATTAAGAGGTCTTGAAAATATATTA |
| | | TCAGAGATTGAAAACACGAATGATTTGCAATCCAGGGTCGAGTCCGTAAATAATGAACTA |
| | | CAGAATATTTTTGACCGCGACAGTAAACAGACAAGATTATTTTGCGAGAATATACTGGCT |
| | | TATTTGATAAAGAATTATGGAAACTCGACTGAAAAACTTATACTACTAATAAATGTTACT |
| | | GAGATGCAATTATATTCACGTTTAGACCAAATGAAGGCCATGAACATTATTTTATATAAT |
| | | ATCCTTTGTAAAGTGGAAGCAAATGAAAACCCACCGTACAGTCCCACATTGGTTACCGCA |
| | | CTAGAAAACCTATTGGCAGCGATCAACAACAGATTTTTTCCAGGGCGTTGCGAAAATTCA |
| | | TTGCACCCAATAGTAATTGAACAACTATTATCATATTTCATTAAAACCGGCAATTTGAAT |
| | | GAAAGCAAAAATTTTTTGGGCCATTTAATAAAAAAGGGTATTTTACCAGAGGCTACTATC |
| | | ATTAATAGATATTTGGAGGCAATTGATGTTCATTTCGATAAAAGCACTAAGATTTTTGAT |
| | | ATAAGATCCAAATTTGCATTCATAGCAGATCTTGCACCCATAATAGAAAATTATGGAACA |
| | | ATAGACCTGTTCAAATTTTTGATACCTATGTGCCGGCATTTCGACGAATTGTGTTCTCTG |
| | | CTAAATATTATTCGAAAATCCAATAACGCAAAACGGGCTGTGGATAGCACCTTACCTATT |
| | | TTTATCAAAAAAGTATTAACATTTACTAAAGATCCAATGATCAACTCCGGAAATCTATCC |
| | | ACTGTTTTCAATATAGTGAGCCCAATATATGGACAAAACGTGCCCAGCGAATTCGTGGAG |
| | | AAATTTATACTGTCTTTTGCACTTCAAGGAAATTACACTATGATGGCTCATATGATAGAT |
| | | ACATATAAGATCAAACTAAGTCATAAATACCAACTACAAATTATTAGGGCTCTCAAAAAT |
| | | AGTGAGAGAAATCACGCGCTTAAGAATACGGGCGCTGTAGGTTATAATAAGGAATTTAAA |
| | | AAGTACTTCATCGAGAAATATTTGAATTGCACTGAACGAGAAGCTTTACGCCCATAA |
| YBR268W | MRPL37 | ATGTTGGCGCGCAGTTTGGGTTACAGGTTGATATCGACCAGTCGTATCTTATATAATAAA |
| | | CCCACGGTTAAGTCAGTGGTGTCGTCGTGTCCTGCGGGAACATCGCTTAACTTAAACATA |
| | | TGGAAGAGCGGTAAAGACGCAGTGGCACTCGAGGACAAGGAGTATCCAAATTGGTTATGG |
| | | AGCGTATTGGATAGTGATCACGTTGTCGAACATGCGGCTGAGGACCCAGAGGGACAGGCT |
| | | CTCTTAAAGAGAAGAAAGAACATAAGGAAGGCGAACAGGCAGCGAATCAAGCAAAACAAT |
| | | TTCTTGAGCCAACTTTAA |
| YHR091C* | MSR1 | ATGTTCGGGATAGTTTATCTGAAGAATCGCAGTTTACTCTGCAAGAACCCCTTTTCTTCT |
| | | TACCCTCGTTATGGCTTCATGCCCTCGTTCGACACCCAGTTCAGCAACCAATTTCGTAAA |
| | | TTAGAAATTAATATTGGACGGAAAAGATACAGCTCAAAAACCTTGAATACAAAATATACT |
| | | GACCAACCCGAAGGCCCAATTTATCCTTTGGATGTACTACGCCTAGACATCTCAAAAGCA |
| | | CTGCATGATATATCCGGAATAGACCACTCGCTAATACTAAATGCGCTTGAGTCGACAAAT |
| | | AGTATGGATAGAGGTGATCTACTGCTACCTCTTCCAAAGATAAAAGTGGCTGACCCTGTG |
| | | GCCGTTGCCAACCGATGGGCCATTGAACTAAGCACGCATGGGTGTATTGGAAAGGTTTGT |
| | | GCAAAAGGGCCATTTCTTCAGTTCTTCCTGGACCAGCGGTATTTGATACAATCAACGGTA |
| | | CCAAATATATTACTTCAAAAGGGGAAATATGGTCAGAAAAAATCTCGTCATCAGAAAAAA |
| | | GTTGTAGTAGAATTTTCATCACCAAATATTGCCAAACCATTCCATGCAGGCCATTTAAGA |
| | | TCGACTATAATAGGTGGATTCCTTTCAAACTTATATGAAGCAATGGGGTGGTCCGTTACA |
| | | CGTATGAACTACCTAGGAGACTGGGGCAGACAGTTTGGTTTGTTAGCAGTCGGTTTTAAG |
| | | AGATACGGCGATGAAAAGACGTTACAAAAGCAGCCAATTCAGCATTTATTCGACGTCTAC |
| | | GTTAAAATCAACATGGACCTTGCCAAGGAAGAGATAAACGGAAACTCTAAGTGTGGGATT |
| | | AGCGGTGAGGCCCGTAGTTTTTTCAAAAACTTAGAAAATGGGGATGAGAATGCCATTAAG |
| | | ATATGGAATAGGTTTCGCTCGTTATCCATCCACCACTATATCCAAACTTATTCACGTTTG |
| | | AACATTAATTTTGATATATTCTCCGGTGAATCACAAGTGTCTAAAGAATCTATGAATGAA |
| | | GCCTTAGATATATTTCGCAAAAACAATCTTGTTAAAGAAATAGACGGTGCCCTTGTCATC |
| | | GATTTAACCCAATGGTCGAAAAGATTGGGCAGAGTTGTTGTACAAAAATCAGACGGAACA |
| | | ACTTTGTACTTGACAAGAGATGTAGGGGCAGCCATAGAGCGTAAAAAGAATCTACATTTT |
| | | GATAAAATGGTGTACGTGATTTCCTCACAGCAAGATTTATACATGTCCCAGTTTTTTATG |
| | | ATATTAAAAAAAATGAATTTTGAATGGGCAAAAGACTTGCAGCATATAAATTTTGGTATG |
| | | GTGCAGGGCATGTCGACCAGAAAAGGAAATGTGGTATTCTTAGATACCATACTAGACGAG |
| | | GCAAGGGATAAAGCTCTGCAAATTATGGAAAATAACAAAATGAAAATATCTCAGGTAGAT |
| | | AATCCACAGCGTGTTGCAGATTTAATTGGCGTATCTGCTATCATTATTCAAGATATGAAA |
| | | TCAAAGCGTATCAATAACTATGAATTCAACTGGAACAGAATGTTGTCTTTCGAAGGTGAC |
| | | ACAGGGCCCTATTTACAATACACACATTCCAGACTTAGATCTTTAGAAAGGACCTCCAGC |
| | | GACTTTACGACGGACATGCTAATACACGCAGATTTTTCAAATTTAAATGAACCACAACTA |
| | | GTTGAGCTTGTGAGGTTGCTAGCGCAGTATCCAGATGTCCTTAGACGAGCATTCGAAACT |
| | | CAAGAGCCGGCCACGATTGTAACATATTTATTCAAAGTTTGCCATCAGGTCTCCTCGTGC |
| | | TATAAAAAAATATGGGTTTCCGGAAAACCTGCAGATATTGCCATACCGCGACTCGCCGTA |
| | | TATTCAGCAAGTAGACAAGTGTTACACAATGCAATGTCGCTTTTGGGACTAGTCCCGGTT |
| | | GATAGAATGTAA |
| YDR296W | MHR1 | ATGAAGGTAAACCATTCAATTTCAAGATTCCGCCCCGCTTCATGGTTCGAAAAGACAAAG |
| | | ATAATCCCACCCCAGGTATACATCTTTAGGAACCTAGAGTATGGACAAGTACTATATTCT |
| | | CAATTTCCCAACTTTTCGCAAACACAGGTGGATAAGCTGTTTGTGAGACCAAACTGGAGC |
| | | AACAGAAAGCCATCATTGAGAAGGGACATCTGGAAATGTATGTGTGTAGTGAACTTGCAA |
| | | AACTATAAGCAGAGTGTCCATCTATACCAGAACCTCTGCCGGTTGAGATACCTCCGTGAT |
| | | GTGGCACAGCGTAAGGAGAGTGACAAGCTAAGAAAAAAGGACTCTAACGGGCACGTCTGG |
| | | TATAGCGGACAGTATAGACCTACATATTGTCAAGAGGCAGTGGCAGACTTGCGGGAGTCC |
| | | TTGTTGAAGGTGTTTGAGAATGCCACACCAGCAGAAAAGCAGACAGTACCCGCCAAAAAA |
| | | CCGTCCATATACTGGGAGGACCCATGGAGGATGGGTGACAAGGACAAACATTGGAATTAC |
| | | GATGTGTTCAATGCTCTGGGGCTGGAACACAAGCTTATTCAGCGTGTGGGGAACATTGCG |
| | | AGGGAAGAAAGCGTTATTCTGAAGGAACTAGCTAAGCTCGAATCACATCCTACAGAGCAG |
| | | ACGGAAGTGTCTTCCCAGTAG |
| YKR006C* | MRPL13 | ATGTCGTCACTGCTTAAATTACATTGTATTAGACCATTGCCGCAAAGGTCGGTCTGGCTC |
| | | TCTGGCTACAAGCAGAAAGCCAGATGTATTCACAGTTCCGCTGCAAACGGGGACTTTATG |
| | | TCATGGTTCAAGAGGAAGAAGCAAGAAGAGCACCAAGAGCCTGTCAAGGACACCAAACAG |
| | | TTGATAAAGGATATTGAAGAAGGCACTAACGAGGCTTCCTCTCAATCCTCTTCCAATAAC |
| | | AAAAACAGATTGGAGCTAATACCAGAAAATTTTATTGGCGAAGGGTCTAGACGGTGTAAG |
| | | AGACAGAAGGAGTTGAAGCTTGCGGTCTCCTCTGCGCCATTCAATCAGTGGTTATCCAGG |
| | | GATAAGATTACGTCTGATAATCAGTTAGATGATATGATATTGCAGGCAACGGAAAAAACA |
| | | TTGGGCAAAGTAGACCAGGACGTCCAATTCTCTGATTTGGTAGCTAAATTTCAGTTCACC |
| | | AAATTTTTGCAATCTAAATCTGGCTATCTAATTCCTGATTATGAACTGACCACTTTAAGC |
| | | ACACCCTTGCAGTTTAAAAGGTATATTAAGGAAAAAATTTTGCCATCTGCCAACGATCCT |
| | | AAACTCGCTTATAAAGAAGCTGAGCCAAATGCTATCCACCCATTTTCTGATAATTACGCG |
| | | TCACCAAACATATATGTTGTTAACGATGTTACTTCTAAAGAGCAAAAAAGTAAATATGAT |
| | | ACTATTATGAAAGAAATTCAAAAGTTAGAGGATGATGCAACAAGAAAAGCTTTGGAAACA |
| | | GCAAGATCCGCTTAG |
| YBR122C* | MRPL36 | ATGTTGAAGTCTATATTTGCGAAGCGATTTGCTTCCACAGGGTCATATCCTGGTTCTACA |
| | | AGAATTACTTTGCCAAGAAGGCCTGCAAAAAAAATTCAACTGGGGAAGTCACGACCGGCT |
| | | ATTTATCACCAATTCAATGTCAAAATGGAGTTGAGTGATGGAAGTGTAGTTATCCGGAGA |
| | | TCCCAATATCCAAAGGGTGAAATTAGATTAATTCAAGACCAGAGAAATAATCCGCTGTGG |
| | | AATCCTAGCAGGGATGACCTAGTCGTTGTGGATGCTAATTCTGGTGGTAGCTTGGACAGA |
| | | TTTAACAAGAGGTATAGCTCTTTGTTTTCTGTCGATTCAACGACCCCAAATTCAAGCTCT |
| | | GAAACAGTTGAACTATCTGAAGAAAATAAAAAGAAAACACAGATTAAAAAGGAGGAGAAG |
| | | GAAGACGTTTCTGAAAAAGCGTTCGGTATGGATGACTATCTATCTCTGTTAGACGATAGT |
| | | GAACAACAGATCAAATCAGGTAAGTTGGCCAGTAAGAAGAGAGACAAGAAGTGA |
| YBR179C* | FZO1 | ATGTCTGAAGGAAAACAACAATTCAAAGACAGCAATAAACCGCACAAGGACTCCACAGAT |
| | | CAGGACGATGATGCCGCCACAATAGTACCACAAACTCTTACTTACTCACGCAACGAAGGT |
| | | CATTTTTTAGGCAGTAATTTCCATGGAGTAACTGATGACCGTACCACTTTGTTTGATGGT |
| | | GAAGAGGGGCGTAGGGAGGATGATTTGTTGCCCTCATTGCGTTCCTCCAACTCAAAAGCA |
| | | CACTTAATTTCTTCACAACTTAGCCAGTGGAATTATAACAACAATAGAGTACTTTTAAAA |
| | | AGATCTATATTAAAGACACAAGCTTTTATGGATCAACTTCAAGAAGAAAATAATATTCGC |
| | | CCGATTTTTATTGCTGCAAATGATGAACGGGAAAAATTACATGTTCTACAACTGAATATC |
| | | AAGCTAGATGGTCAGTACAATACCAAAGAAAAGAACGGTTTCAACATTGAGAAAAAAGCT |
| | | TTATCAAAATTATTTCACTCTCAGATCGTATCAGTGACAAATCATTTAAACGCTTTGAAA |
| | | AAAAGAGTGGATGACGTTTCATCTAAAGTATTCATCACAGGTGATGTAAATACTGGTAAA |
| | | TCAGCTCTTTGCAACTCTCTATTAAAGCAGCGTTTGCTACCTGAGGATCAGCTACCATGT |
| | | ACCAATGTATTTTCCGAAATACTAGAGGCTCGGGAAAATGACGGCATAGAGGAGGTTCAT |
| | | GCCATACCACTAAACATTGCTCCGACTCTCAAGGAAGCCATTGATATGTATTCAATACAA |
| | | AATCCCAAGACTTATGAAATACACACTTTGAAAGAACTTCCCGATTTGGTTCCCCAAAAC |
| | | GGAAAATATGCTTTACTGAAGATTTACATAAAGGACGATAAAAGGCCTGCCTCTACTAGT |
| | | CTATTAAGGAATGGTACTGTTGATATTTCATTAATTGACTCCCCTGGGTTGAACATGGAC |
| | | TCGTTACAAACCGCCGAAGTAATGTCCCGACAAGAAGAAATCGACTTAGTTATATTTGTT |
| | | GTCAATGCAGAGAACCAGCTAACACTATCTGCCAAGGAGTTTATTTCTTTAGCATCTCGT |
| | | GAGAAAAAATTAATGTTTTTTGTTGTGAAAAAATTTGACAAAATCAGGGACAAACAACGT |
| | | TGTAAGGAGTTGATCCTAAAGCAAATCCGTGACCTGTCCCCAGAAACATATAAACGTGCA |
| | | GCTGATTTTGTCCACTTTGTTTCCAAAAACGGAGATGAATTGCCACATTATCATAATGAA |
| | | AATGACAATGAAGATCATGGTGACCGAAAACCTGACGACGACCCATATTCTAGCAGCGAT |
| | | CCCGATCCGGATTTTGACAGTCTGGAAGATTCTCTACGCAATTTTGTTTTGAAGAAAAGA |
| | | TCACTTTCTAAATTGTTACCTGCCAAGACTTATCTATCGAAATTATTAAGCGATATAATA |
| | | ATGATCTCCAAATCTAATATGAAAATGTATAGTGAAGAAGAAATAAAAATTAACGAACAA |
| | | CTGGAAACATTGAGACCTGAAATTTTGAGCGCTAGAGCAAAGTGCAATGATTTAACAACT |
| | | TCCGTTGATCAAATGGCCGAGCAAACAATTACAATGACTTATAACAATACTAAAGAAGCA |
| | | CTTCTCAATGCGTTGGATGTTCCGCTACACGAATATCCGAAATATCAGGGCCTCGGTCAA |
| | | ATTTATGACTTCATATTTTCAACAGAAGCGTTCATAGCAAATCAGATTGATGAGTCAATA |
| | | GGATCAAGTGAATTATTTGCCAAGCAAAAGACAGATTTACTGGTAAAAAAAATTTATGAG |
| | | ATTGGAAAAAATGAACTTGGTGATGATTTCATGTGTGAACGTGTTTTCAGAAGCGAACTA |
| | | ATGTTTAGAAAAAGGAAGCATTTGATAGGAAAAAGGCTTAAAGTATCTTTGTCAATAACT |
| | | GATTTGTTTGCTCCAACTTGGAAAGGCTTTTTGTCTTACTTAAGCTGGCAGAAACCTGTC |
| | | ACAGCACCACTGCCGGATATAGAAGGACAAACTAACGAGGGTCAGATTGGCCTAATGAAG |
| | | TATTTGGGTTTGAAAAATTATCCTTTGACACAATATTGGTCAAGACCATCATTGCTCTTC |
| | | ACATCAAAAATTCCCACTCTAACGCTGTACTTTCTAGGAAGTACAAAGGTTGTGGGGAAC |
| | | ATTATATTAAACGGCATTAAATTATCGTCGTGGAGCTCACTCAAGAAGTTATCAGTTCCG |
| | | GTTATAGTGGTAGGTTCCTTGTTGGGACTTACGTATTTAATTCATGACCTCCCCCGTGCC |
| | | TTACCAATGAATCTTTCCATTAAATACAAAAGAAAGTTGCAAGAACTGGATTACATTCAT |
| | | CTTAACGCTCAAAGAACCTCAAATGAAGTACGTGATGTTTTGCGTGTACCTACACGTGAA |
| | | ATTCTAAGGTCGTGTGAAATAATTATGGATAAAAAACAAATCACTAAGAAAGAATTGGAA |
| | | AACAAAAAAGAAAGTAACTTGTTGTCAATCAAATTCTTCCAATCTCTATACGAAGGAACC |
| | | GTGGCTCAAAAATTGATGGTGGAAGAAATAAATTTAGACATCGATTAG |
| YBR126C* | TPS1 | ATGACTACGGATAACGCTAAGGCGCAACTGACCTCGTCTTCAGGGGGTAACATTATTGTG |
| | | GTGTCCAACAGGCTTCCCGTGACAATCACTAAAAACAGCAGTACGGGACAGTACGAGTAC |
| | | GCAATGTCGTCCGGAGGGCTGGTCACGGCGTTGGAAGGGTTGAAGAAGACGTACACTTTC |
| | | AAGTGGTTCGGATGGCCTGGGCTAGAGATTCCTGACGATGAGAAGGATCAGGTGAGGAAG |
| | | GACTTGCTGGAAAAGTTTAATGCCGTACCCATCTTCCTGAGCGATGAAATCGCAGACTTA |
| | | CACTACAACGGGTTCAGTAATTCTATTCTATGGCCGTTATTCCATTACCATCCTGGTGAG |
| | | ATCAATTTCGACGAGAATGCGTGGTTGGCATACAACGAGGCAAACCAGACGTTCACCAAC |
| | | GAGATTGCTAAGACTATGAACCATAACGATTTAATCTGGGTGCATGATTACCATTTGATG |
| | | TTGGTTCCGGAAATGTTGAGAGTCAAGATTCACGAGAAGCAACTGCAAAACGTTAAGGTC |
| | | GGGTGGTTCCTGCACACACCATTCCCTTCGAGTGAAATTTACAGAATCTTACCTGTCAGA |
| | | CAAGAGATTTTGAAGGGTGTTTTGAGTTGTGATTTAGTCGGGTTCCACACATACGATTAT |
| | | GCAAGACATTTCTTGTCTTCCGTGCAAAGAGTGCTTAACGTGAACACATTGCCTAATGGG |
| | | GTGGAATACCAGGGCAGATTCGTTAACGTAGGGGCCTTCCCTATCGGTATCGACGTGGAC |
| | | AAGTTCACCGATGGGTTGAAAAAGGAATCCGTACAAAAGAGAATCCAACAATTGAAGGAA |
| | | ACTTTCAAGGGCTGCAAGATCATAGTTGGTGTCGACAGGCTGGATTACATCAAAGGTGTG |
| | | CCTCAGAAGTTGCACGCCATGGAAGTGTTTCTGAACGAGCATCCAGAATGGAGGGGCAAG |
| | | GTTGTTCTGGTACAGGTTGCAGTGCCAAGTCGTGGAGATGTGGAAGAGTACCAATATTTA |
| | | AGATCTGTGGTCAATGAGTTGGTCGGTAGAATCAACGGTCAGTTCGGTACTGTGGAATTC |
| | | GTCCCCATCCATTTCATGCACAAGTCTATACCATTTGAAGAGCTGATTTCGTTATATGCT |
| | | GTGAGCGATGTTTGTTTGGTCTCGTCCACCCGTGATGGTATGAACTTGGTTTCCTACGAA |
| | | TATATTGCTTGCCAAGAAGAAAAGAAAGGTTCCTTAATCCTGAGTGAGTTCACAGGTGCC |
| | | GCACAATCCTTGAATGGTGCTATTATTGTAAATCCTTGGAACACCGATGATCTTTCTGAT |
| | | GCCATCAACGAGGCCTTGACTTTGCCCGATGTAAAGAAAGAAGTTAACTGGGAAAAACTT |
| | | TACAAATACATCTCTAAATACACTTCTGCCTTCTGGGGTGAAAATTTCGTCCATGAATTA |
| | | TACAGTACATCATCAAGCTCAACAAGCTCCTCTGCCACCAAAAACTGA |
| YLR312W-A | MRPL15 | ATGGAAAATAGCATGATGTTTATATCGCGTTCCTTGCGGAGACCCGTTACTGCTTTGAAC |
| | | TGCAATTTACAAAGCGTTAGAACAGTCATATACCTTCATAAGGGTCCGAGAATTAATGGT |
| | | TTGAGACGAGATCCAGAGTCGTACTTGAGAAATCCTAGCGGGGTACTCTTTACTGAAGTT |
| | | AACGCAAAAGAGTGTCAAGACAAAGTTAGATCCATACTACAATTGCCCAAGTACGGAATA |
| | | AATTTATCAAACGAACTGATTTTGCAGTGTTTAACACATAAGTCGTTCGCACACGGGTCA |
| | | AAGCCATACAACGAAAAACTGAATCTGCTAGGAGCACAGTTTTTGAAACTACAGACGTGC |
| | | ATTCATTCGTTAAAGAATGGTTCGCCGGCCGAGAGTTGCGAGAACGGGCAACTTTCTTTG |
| | | CAGTTCAGCAATCTGGGAACAAAATTCGCTAAGGAATTAACGTCCAAAAACACAGCATGC |
| | | ACATTCGTTAAATTACATAATCTAGACCCTTTCATATTTTGGAAGATGAGAGACCCAATA |
| | | AAAGATGGCCACATAAATGGTGAAACCACGATTTTTGCTAGTGTCTTGAACGCATTTATT |
| | | GGTGCAATTCTGTCAACGAATGGATCTGAAAAAGCAGCTAAATTTATCCAAGGAAGTTTG |
| | | CTGGATAAAGAAGATCTTCATTCCTTGGTTAACATTGCTAATGAAAATGTAGCATCTGCA |
| | | AAGGCAAAAATATCCGACAAGGAAAATAAAGCATTTCTGTGA |
| YKL003C* | MRP17 | ATGCTTTATGAGCTGATCGGACTTGTTCGCATAACCAACTCCAATGCGCCAAAGTTGGAA |
| | | GCAAAAGAATTGTCTTCCACAATCGGGAAACTGATTATTCAAAACAGAGGTGTGGTGAGG |
| | | GACATTGTACCCATGGGCATAAGGTATCTTCCTAAAATAATGAAGAAAGACCAGGAAAAA |
| | | CATTTTCGAGCATATCACTTTTTAATGCTGTTTGATTCATCAGCTGCGGTACAGTCTGAA |
| | | ATTCTAAGAACTTTAAAGAAAGATCCCCGTGTCATAAGATCATCCATCGTTAAAGTTGAT |
| | | TTAGATAAGCAGCTGGATAGAGCCTCATCGTTACACCGTTCTTTGGGGAAGAAGTCTATT |
| | | TTAGAATTAGTGAATGAAGATTATCAATCCATTTAG |
| YLR270W | DCS1 | ATGTCTCAACTGCCAACAGATTTTGCTTCATTGATCAAGAGATTCCAATTTGTTAGTGTC |
| | | CTCGATTCCAACCCTCAAACCAAAGTTATGTCCTTATTGGGAACCATTGACAACAAGGAT |
| | | GCTATTATCACGGCTGAAAAGACGCACTTTCTATTTGACGAAACCGTGAGAAGACCCTCT |
| | | CAAGATGGCCGCTCCACTCCAGTTTTATATAATTGTGAAAACGAATATTCTTGCATCAAC |
| | | GGAATTCAGGAATTAAAGGAGATTACTTCCAATGATATTTACTACTGGGGTTTGTCAGTG |
| | | ATAAAGCAGGATATGGAGTCGAACCCTACCGCCAAGTTGAATTTAATTTGGCCTGCTACT |
| | | CCAATTCATATCAAGAAATATGAACAACAAAACTTTCATTTAGTCAGAGAGACTCCTGAG |
| | | ATGTACAAGAGAATTGTTCAGCCTTACATCGAAGAAATGTGTAATAACGGTAGACTTAAA |
| | | TGGGTAAACAACATTCTTTATGAAGGCGCTGAGTCAGAAAGGGTCGTTTATAAAGATTTT |
| | | TCTGAGGAGAATAAGGACGACGGCTTCCTAATTTTACCTGATATGAAATGGGATGGCATG |
| | | AACTTAGACTCTTTATATCTAGTTGCGATCGTCTACAGGACCGATATCAAGACAATAAGG |
| | | GATTTAAGATATTCTGACAGACAGTGGTTAATCAACTTGAACAACAAGATTAGATCCATT |
| | | GTTCCTGGTTGTTACAACTATGCTGTCCATCCGGATGAGTTGAGAATTTTGGTTCACTAC |
| | | CAACCTTCTTATTATCATTTCCACATTCACATCGTTAACATAAAGCATCCCGGTTTAGGT |
| | | AATAGTATTGCCGCGGGCAAGGCCATCTTGTTGGAAGACATCATTGAAATGTTAAATTAT |
| | | CTAGGACCAGAAGGCTACATGAACAAGACAATCACCTACGCTATTGGTGAAAATCATGAT |
| | | TTGTGGAAAAGGGGTCTGGAAGAAGAACTCACTAAACAATTGGAAAGAGATGGTATCCCA |
| | | AAAATACCCAAGATTGTGAACGGTTTTAAATAA |
| YFR032C-A* | RPL29 | ATGGCTAAGTCTAAGAACCATACCGCTCACAACCAAACCAGAAAGGCTCACAGAAACGGT |
| | | ATCAAGAAGCCAAAGACCTACAAGTACCCTTCTTTGAAAGGTGTTGATCCAAAGTTTAGA |
| | | AGAAACCACAAGCATGCCCTACACGGCACTGCTAAGGCTTTGGCTGCTGCCAAGAAATAA |
| YPL271W | ATP15 | ATGTCTGCCTGGAGGAAAGCTGGTATATCATATGCTGCATATTTGAATGTGGCCGCTCAG |
| | | GCTATCCGTTCTTCATTGAAAACTGAATTACAAACCGCTAGTGTTCTTAACAGATCGCAA |
| | | ACAGATGCTTTTTATACCCAATATAAAAATGGCACTGCAGCTTCTGAACCCACTCCAATA |
| | | ACAAAATAG |
| YPL104W | MSD1 | ATGTTGGCCCGTTCCAGGGTGTGCTTGCAGACAATCACTAGACGGTTGGCAGACTTTCCA |
| | | GAAGCCAATGCTATTAAGAAAAAATTTCTCTTTAGGAAGGACACCTCGACCATCAAACAG |
| | | TTAAAAGGACTGTCTAGCGGCCAGAAAATAGTACTCAATGGATGGATAGAGCAGAAGCCA |
| | | AAAAGAGTTGGGAAAAATTTGATCTTCGGACTTTTAAGGGACTCTAACGGTGACATTATC |
| | | CAGTTGGTTGATAACAAATCGTTGTTGAAAGGCTTTACTTTAGAGGATGTGGTTCAAGCC |
| | | GTAGGTATACTCTCTTTGAAGAGGAAGCTATCAAATGAGGACGCAGATGAGTACGAAGTG |
| | | CAATTGGAGGATATTACTGTGTTAAATGCATCTAATAAAAAACCAGCGCAAATGCAGGAT |
| | | TTTAAATTGTCAGCCATATACCCACCTGAGTTCCGCTATTTGCAATTGAGAAATCCCAAA |
| | | TATCAAGATTTTTTAAAGAAGAGGTCATCTATCTCTAAGGAAATAAGAAACTCCTTCAAC |
| | | AATTTTGATTTTACGGAGGTCGAGACCCCAATGTTATTTAAAGCTACCCCAGAAGGCGCA |
| | | AGAGAGTTTCTGGTTCCAACAAGGACAAAGAGATCCGATGGTAAACCATCGTTTTATGCT |
| | | CTAGATCAGAGCCCTCAACAATACAAGCAACTCTTAATGGCTAGTGGTGTCAACAAATAT |
| | | TATCAAATGGCAAGGTGCTTTAGAGATGAAGATTTAAGAGCAGACAGGCAGCCTGAGTTT |
| | | ACACAGGTTGATATGGAAATGGCCTTTGCTAATTCTGAAGATGTCATGAAAATCATAGAA |
| | | AAGACAGTTTCTGGGGTATGGAGTAAATTTTCCAAAAAACGAGGATTATTGACTTTAGAC |
| | | AGTAAGGGTACATTAGTGCCTGCGAAAAAGGAAAACGGCACAGTATCTATCTTTCGTATG |
| | | ACCTACGAACAAGCCATGACCTCATATGGTATTGACAAGCCAGATTTGAGAGCGCCAGAT |
| | | TTGAAGATTATCAATTTAGGCGAGTTCAATGCCTTTAGTCATTTGAACAAAAAATTTCCC |
| | | GTTTTTGAAGTAATTATTCTAAGAAGTGCCTTTTCAAATATGGAAGAGTACAAAGAACGA |
| | | TGGTCGTTTCTGACAAATAACAGTAATTACAATTATAGAGTTCCAATAGTGCTACCAATT |
| | | GAAAATGACGAACAAGCTAATTCAAATTGGTTTGAGAATTTTCATGCAATTGCCACGTTT |
| | | GAAAACCCACATCTAATAACCAAATTTCTGAAACTGAAAAAAGGTGACATTGTATGCGGT |
| | | TGTACGAGAGAGCCAAACCATTCCATTTTCGAGAATCCTACTCCCCTGGGAAGATTGAGA |
| | | CAGTTGGTGCTACAAAGTGAGCATGGGAAAAATATCTATCATGCTGTCAATAAGGATGTT |
| | | GCCTCATGGATTGTGGATTTCCCGTTATTTTCTCCCGTTATAATTGAAGATAAGTCTGGT |
| | | AAAAAAGAAAAGCTTGCATATCCGGAGTACGAAAAGGATAGACTATGTTCCACGCATCAT |
| | | CCTTTTACTATGGTGAAGCTTAAAGACTACGAAAAATTAGAAAAGACGCCGGAAAAGTGC |
| | | TTGGGTCGGCATTATGACCTCGTAGTTAACGGTGTGGAACTTGGTGGTGGCTCAACAAGA |
| | | ATTCACGATCCAAGATTACAAGACTATATTTTCGAAGATATCCTCAAAATAGATAATGCA |
| | | TATGAACTATTTGGCCATTTACTGAATGCTTTTGATATGGGAACACCGCCACACGCTGGA |
| | | TTTGCTATTGGTTTTGATCGTATGTGCGCTATGATCTGTGAAACTGAGAGTATAAGGGAC |
| | | GTAATCGCCTTCCCAAAAAGTATTACCGGGGCTGATTTGGTTGTCAAGAGTCCAAGTGTG |
| | | ATACCTGAAAGCATTCTGGAACCTTACAATATCAAGTATAGTAATTCAAAAAAATGA |
| YDR509W | — | ATGATGTTCACCTTCATATTCCATATTTTCAATGGTTTTTTCCACTGTTTTTTCAAAATA |
| | | TTCTACTTCATTTTCCGGTTCTATAGAGCCAATTTGTTTTTCCTTTGGTTGTATTTCCTT |
| | | CATCTCGTAATGGGAAACATCGTCAAGGTCGTTACGATTCATATTCATATTAGAGCTTCC |
| | | CTCATTATTCCGCCCATGGCGTCTATTACCAAGCGTCATAATGTGCAATATTTGATATTA |
| | | TATAAGCTACTTGAGAAAGCGATAGTTTTTTTTTCTTACACAAAAAAAAAAAAACATAAA |
| | | GCACCTATAACTCTCAACTTTGAAGAAGCACGAAAGGAATATGTTTAA |
| YNL284C* | MRPL10 | ATGAAAGCGGAAAGGCAAACAGGATTAAGAAATAGTTTTACAACCGTAATCGGTCGCAAA |
| | | CTAATAAACACTTTTGTTCCGTCCATGATGTTAACTAGCGTAGCTGGTAACGATATTTTT |
| | | TTCCGAGGTTTATTCAAATCGCCGGTATTAGCATTTCAAAGTTATCGTTATGTTTCGATT |
| | | TTGGGACAATTGAAACCGTCAGATGGTTCTACAAAGTCTTTCAAAAGACTTGGGCGTGGT |
| | | CCTTCCAGCGGTTTAGGTAAGACATCAGGGCGTGGTCAAAAGGGTCAAAAGGCGCGTGGT |
| | | AAAGTAAAATCGTGGTTTGAAGGTGGTCAAACTCCAATTTACAAGCTATTTCCTAAAATT |
| | | GGATTTACCAACGTTGGCGCCAAACCATTAAAGGAATTAAACTTGAAACGCATTCAATGG |
| | | TTTCATGATAAAAATAGGCTTCATTTGCAACCAGGCGAGGTTTTAGACATGAATAAGATG |
| | | AGAAAGCTTGGTCTGGTAACTGGTCCCATAAAGTACGGAGTTAAAATTCTAGCCAGCGGA |
| | | AAGTTCCACTACAATCTACCGATTGCTTTGGAGGCTTCTAGAGCATCCGCTAAGGCTATT |
| | | GCGGCTATTGAAAAAGCAGGAGGTAAGTTTACAGCTCGTTACTACACACCGTTGGGTCTA |
| | | AGGGCACATCTCAACCCCCAGTGGTTTCTTGAGAAGAGGGGAAGAGTACCACTACAGGCT |
| | | AGACCCACGAAAAGAAGGGATATTGATTTCTATAGTAAAGAAGAGAAAAGAGGTTACCTA |
| | | GTAATGGAAAAAGATAAACTCTTACAAGATATCAAAGAAGCTCAAAATAAGGGTTCTAGA |
| | | CACTTTTTAAAACAGAATGTAAAAAAGAGCTCATTAGAAATAGAGCTAGAAGAACTGTCT |
| | | CCAGAGAAGGATTGGGTGCCAGTTGTGTCTAATTCTAAAGTCATGAACATCAAGGCTTTG |
| | | GACCACTAA |
| YNL005C* | MRP7 | ATGTGGAATCCTATTTTACTAGATACTTCTAGTTTTTCATTCCAAAAACATGTGTCTGGT |
| | | GTGTTCCTTCAAGTGCGTAATGCCACCAAAAGGGCTGCGGGCTCGAGAACAAGTATGAAG |
| | | GATTCTGCAGGAAGAAGGTTAGGACCAAAAAAATATGAAGGCCAGGATGTTTCCACAGGT |
| | | GAAATTATTATGAGGCAAAGAGGAACAAAGTTTTATCCTGGAGAAAACGTTGGTATAGGG |
| | | AAAGATCATTCCATATTCGCCTTAGAACCTGGTGTTGTTCGTTACTATCTTGATCCTTTT |
| | | CATCCTAAAAGAAAATTCATTGGTGTTGCCTTGAGGCGTGATTTGAAACTGCCATCTCCT |
| | | CATTTTGAGCCTACCGTAAGGAGGTTTGGCCGTTTTGAGTTAACAAATAAAAGGGCAGCA |
| | | TATAAGGAAGAAAATTCTATTTCTAGAAAAGACTATCTCGCAAAACCTAATATTTTGAAG |
| | | CAGTTAGAGGTCAGGGAATCTAAAAGAAAGGAGTTACAAGACAAGTTGAGTAAGGTTCTT |
| | | CGGGATGAACTTAAGTTAGATATAAAAGACATCGAATTGGCAACGTCATACTTAATTCGT |
| | | GTAAGGGCTTCCCTGAAGAATGGATACCCCATAGAAGATGCAAGGTTCAATAGTAGATAT |
| | | TATTTAAAAGAAGAAGAGCGTTTGAAGGCAAGGAGAGAAAGCTGGACGAATGAGAAATTG |
| | | TCGGAGAGTCTATCCAAGATTGATGAGTGTAGTGATCTTTTGAACTCATCTACATCTTTC |
| | | AATAACAAGCTAGAGCTACACCAGTATATTTCGGAACAAGAAAAACAAGCTTTGAAGGCA |
| | | AAACTATTAGAGGACCTAGAAAAATCCCAACACTTAGAAACCAAAAAAGACAAGAATTAT |
| | | ATTAAGGCGCTTTTCAAGGACGCTTGTAATTTTTTGACTTTATCTGAAGAAGTACATTTG |
| | | CGTAGGAAATACTTAAAGTCGGTCTTCCCCGAAACAGATAGTACGGTTGAAACCAAAAGC |
| | | GGGAAGAAGTCAATTGTGTCTAGACGTTTTGATTACACCAAAAATAAAGTTGAAGTCATT |
| | | GCTAGAAGTAGGCGGGCTTTTTTGAGCAAGCTTTGA |
| YMR267W | PPA2 | ATGAATTTATTACGAATGAATGCTTTAACAAGCAAGGCTAGGTCAATAGAACGGTTAAAA |
| | | CAAACATTGAATATTTTATCCATTCGTAATCATAGACAATTCAGCACCATTCAACAAGGC |
| | | AGCAAATATACCCTCGGGTTTAAGAAATACTTGACATTATTAAATGGAGAGGTTGGTTCT |
| | | TTTTTCCATGATGTCCCTTTAGATTTGAATGAACATGAAAAGACCGTAAATATGATTGTT |
| | | GAAGTACCAAGATGGACAACTGGAAAATTTGAAATATCCAAAGAACTTAGGTTCAATCCG |
| | | ATCGTTCAAGATACGAAGAATGGCAAGCTTAGGTTCGTGAACAATATCTTTCCCTATCAC |
| | | GGCTATATACACAACTATGGTGCTATTCCCCAAACATGGGAAGATCCCACAATTGAACAT |
| | | AAACTGGGGAAGTGTGACGTGGCTTTGAAAGGTGACAATGATCCTCTTGATTGTTGCGAA |
| | | ATCGGTTCGGATGTTTTGGAAATGGGTTCCATTAAAAAGGTCAAGGTATTAGGATCACTC |
| | | GCTTTAATTGATGATGGAGAATTGGATTGGAAGGTCATAGTGATCGACGTAAATGATCCT |
| | | TTATCCTCTAAAATAGATGACTTGGAAAAAATTGAGGAGTACTTTCCTGGGATTCTAGAT |
| | | ACGACTAGGGAGTGGTTTAGAAAGTATAAAGTTCCAGCAGGGAAGCCATTAAATAGTTTC |
| | | GCCTTTCATGAACAGTATCAAAATTCCAATAAAACCATCCAAACTATAAAGAAATGTCAC |
| | | AATTCGTGGAAAAACCTCATTTCAGGATCACTCCAGGAGAAGTATGACAACCTGCCTAAT |
| | | ACAGAAAGGGCTGGTAATGGTGTCACACTTGAAGATAGTGTTAAACCCCCTTCTCAAATA |
| | | CCGCCTGAGGTGCAAAAATGGTATTACGTTTAA |
| YPR116W | — | ATGGGTTTACCTAAATCGGCATATAAGAAGTTACTCATAGATTGTCCAACGAGAGTCATT |
| | | AATAAAAACTGTGCCCAACGAGTCAAAGATGTTTCTCCGCTCATTACAAACTTCGAGAAA |
| | | TGGTCGGATAAGCGCAAGAAGCTCTATTTCAAAGATGAAGAGGAAATGGTGGGGCAGTTC |
| | | CATTTGGAAAATTTCAATTTAAAAAATAACTTATATGGTAGGCTTTTAGCTTCTCCAATG |
| | | AGAGCAGAAAAAATCTCAAAATTAAAATCGTGTAGAGAATTACTAATTCCATTGAAAGTG |
| | | GTACCATCAACTGGCAAAGATCAGCATGCTGATAAAGATAAACTAAAACTGGTACCTACA |
| | | CTAGATTATTCCAAATCATACAAAAGCTCGTACGTATTGAATTCAGCCTCCATTGTACAA |
| | | GACAACTTGGCGGCTGCGACGTCATGGTTCCCAATCTCGGTATTGCAAACATCAACTCCA |
| | | AAGAGCCTAGAAGTTGATTCCTCTACATTTATAACGGAATACAATGCTAACTTGCACGCA |
| | | TTTATAAAAGCAAGACTTTCAGTGATACCGAATGTTGGACCTTCTTCGATAAATCGTGTT |
| | | TTACTCATTTGTGACAAGAGAAAAACACCCCCGATTGAAATACAAGTAGTTAGTCATGGT |
| | | AAAGGGCTACCAATTACACAGTCTGTCTTTAACCTTGGTTATCTCCACGAGCCAACTCTA |
| | | GAAGCTATCGTGAGTAAAGATGCAGTTACTAATGGAATTTACCTGGATGCCGACAATGAC |
| | | AAGGATCTCATCAAACACCTTTATAGTACATTATTGTTTCATTCAGTTAACTGA |
| YCR003W | MRPL32 | ATGAATTCTTTGATTTTTGGTAAACAATTAGCATTTCACAAAATTGTGCCTACCACTGCA |
| | | ATTGGGTGGTTGGTACCGCTAGGAAATCCTTCACTGCAGATTCCAGGCCAAAAACAACTG |
| | | GGATCTATCCACCGTTGGTTGAGAGAAAAGCTACAACAAGATCATAAGGACACTGAAGAT |
| | | AAAGATTTTTTCTCTAATAATGGTATTCTACTAGCAGTTCCTAAAAAAAAAGTATCACAC |
| | | CAAAAAAAAAGGCAAAAACTTTACGGTCCAGGTAAGAAGCAATTGAAGATGATTCACCAT |
| | | TTGAATAAGTGCCCATCATGCGGCCATTATAAGAGAGCCAATACACTGTGTATGTATTGT |
| | | GTTGGACAAATAAGTCATATATGGAAAACGCATACCGCTAAAGAAGAAATTAAGCCGAGA |
| | | CAAGAGGAGGAACTTTCCGAACTAGACCAAAGAGTCCTATATCCTGGTAGAAGAGATACC |
| | | AAATATACCAAGGATTTGAAAGATAAAGATAACTATTTGGAACGTCGCGTTCGGACTTTA |
| | | AAAAAGGACTAG |
| YCR071C* | IMG2 | ATGATCAGTTCGTGTGTTACTAGATGTTTTGGTAGGGGTAAATGCCTTCCAGGGCCTGCC |
| | | ACTGCCTCGATATACCAAACGATAAGATGTATATCCACTAATTCAAATAAAGCTGCTGAG |
| | | GCGCCAATATTTCCAAAGCTGGAAGACGTGAAGATGCATGAGCTCATAGGAAACAACAAT |
| | | TTTGGTAAAAAGACCTACTACGTGGAGAGAAGCAGGACCGGAAATCTACCGGTGTATTCC |
| | | GCTTATAAAAATGGAGGTAACAAGATTATCACGGAGATCAGAAAGATTGAAGGAGATGTA |
| | | ATTCAACTAAGAAATGACTTGCAGGAGCAACTGCCTTTCATACCCAAAAAATCATGGTCT |
| | | GTGGTGATGCAATCGAAAAAAATTATTATCAAGGGCAATGCAGTAGAGGCTGTCAAAAGG |
| | | GTATTAACCAAGAAATTTTAG |
| YOL009C* | MDM12 | ATGTCTTTTGATATTAATTGGAGTACATTGGAATCTGACAATAGACTGAATGATCTTATA |
| | | AGAAAACATTTAAATTCATATTTACAAAATACGCAACTACCAAGCTACGTAAGTAATCTT |
| | | AGGGTACTAGATTTTGATCTGGGAAAAGTTGGTCCTGCTATAACACTGAAAGAAATAACA |
| | | GATCCCCTAGATGAGTTTTATGATTCAATTAGAGAGGAGGCTGATCAAGAGACGGAGGAA |
| | | AATAATGACAACAAGGAGGACAGCGAACATATTTGTCCTGATCGTACTATAGCTAATCAT |
| | | GAAGGACCTAAAGATGATTTTGAGGCGCCTGTGGTGATGCCATCCCCCAACGATATACAA |
| | | TTTTTACTAGAGGTCGAGTACAAGGGTGACTTACTCGTTACGATTGGTGCCGACCTTGTG |
| | | CTGAATTATCCCGTAGAAAAATTTATGACTTTGCCAGTAAAACTTTCTATCAGTGATATC |
| | | GGTCTCCATTCCCTTTGCATTGTTGCATGTTTATCAAAGCAACTATTTCTTAGCTTTCTG |
| | | TGTGATGTTAGTGATCCTGCACTGGATGACAACCAAACTGTTTTAGATCCTAAGGGGCCA |
| | | ATTCTGGCAGCTACTAAGCCATTGGAGCGAATATCCATTGTGAGAAGTATGAAGATTGAA |
| | | ACCGAAATAGGGGAACAATATCAGGGACAGGGCTCAGTATTAAGAAGTGTTGGAGAACTA |
| | | GAACAGTTTTTGTTTACGATATTCAAAGATTTTTTAAGAAAGGAGTTAGCATGGCCAAGT |
| | | TGGATTAATCTGGATTTCAACGATGGTGATGAGTAA |
| YER058W | PET117 | ATGAGTAGGGCTAGCAAGATAACGTTTGCAGCTTCCTGTCTGATAACGGCAGCGACGGTA |
| | | GTGGGCGTGCATTATGTGCAAGAAATGGAAAGGGAAACTTTGCATCAGGGTCCGATAAAA |
| | | GATGCTAAACGAGTCGAAGAAAAGAGGTTGAGAAAGACAAACGGAGTTGCATCATTAGAT |
| | | CCCACAAAAGAAAGGAAAAGGTACTTCAATATGAGTGAACACGAGGAACAAAAAGAGTTG |
| | | CGAAAGAAGTATGAGACCATGCAACCGCTTAGTGGAGAAGTTGTGACCAAAGATGGAGAG |
| | | GTGGTTAAAGAATCTAAGAAATAA |
| YCR046C* | IMG1 | ATGTGGAGCAGGAACGTCAGATTGCTTGGATCATGGACAAGGTCCTACATGGTCCCCGCC |
| | | ACCAAGAGAAAAACCATCCCCGTGTACCCACCTGTGCAGCGCATAGCTTCGTCGCAGATT |
| | | ATGAAGCAGGTGGCCCTCTCAGAAATAGAGTCTCTGGATCCCGGGGCCGTTAAGAGGAAG |
| | | CTCATCAGTAAAAAGAACAAGGACCGCTTGAAGGCAGGCGACGTGGTCCGGATTGTGTAC |
| | | GACTCGTCCAAGTGCTCGTACGACACCTTTGTTGGCTACATCCTTTCCATAGACCGCAAA |
| | | CAACTGGTGCAAGACGCCTCGTTGCTGTTGCGGAACCAGATAGCCAAGACGGCCGTCGAG |
| | | ATTAGAGTGCCATTGTTTTCGCCGCTGATCGAGAGAATCGACTTGCTAACCCCCCACGTC |
| | | TCGAGCAGACAAAGAAACAAACACTACTACATCAGAGGTACAAGGTTGGATGTCGGCGAC |
| | | CTCGAGGCAGGTCTAAGAAGAAAGAAATAG |
| YDR462W | MRPL28 | ATGCTGGCACAAACATTCAAAAAACCACACAGAGCCGTTCTAGAACAGGTATCTGGGACC |
| | | ACGGTCTTCATCAGAAATAAGAGAACAAAGAGCAAGAGCTCACTGTCACCTTTGGCACAA |
| | | AGGGTCGTCACGCAGTTGAGTGTGATGTCTGCAAGCAGAAAGCAGCCCAAGTTGCTGAAG |
| | | CTGGCGCGTGAAGACCTGATTAAACATCAGACCATTGAGAAGTGTTGGTCAATTTATCAG |
| | | CAGCAACAACGGGAGCGCAGAAATTTACAGTTGGAATTACAATATAAGAGCATTGAGAGA |
| | | TCTATGAACCTTCTACAAGAACTCAGCCCTCGTCTGTTTGAGGCTGCCAATGCTTCCGAG |
| | | AAGGGCAAGCGATTCCCGATGGAAATGAAGGTGCCCACTGACTTCCCCCCAAATACGTTA |
| | | TGGCATTATAACTTCCGAAAATGA |
| YPL052W | OAZ1 | ATGTATGAAGTAATACAGAAAAGGAAAACAAAAATAATAAACGTTTTACAGAGTCCTGAA |
| | | CTCATGAGGCTCATAGAGGACCCATCAAATCTGGGTATTTCTTTACATTTTCCAGTAAGT |
| | | TCACTGCTAAAAAGTAATAAGTGCACACCAATGCCTAAACTTTCTACGTATAGTTTGGCT |
| | | AGTGGGGGATTTAAGGATTGGTGCGCGGACATCCCTCTAGACGTTCCACCAGAGATTGAT |
| | | ATCATCGATTTTTACTGGGATGTTATTTTATGCATGGAATCTCAATTCATATTAGATTAC |
| | | AATGTTCCGTCAAAAAATAAGGGGAACAATCAGAAGTCTGTTGCTAAGCTGTTGAAAAAT |
| | | AAGCTTGTAAACGATATGAAAACTACGTTAAAAAGACTAATTTATAATGAAAATACCAAG |
| | | CAATATAAAAATAATAATAGCCACGATGGTTACAATTGGAGAAAACTAGGCTCGCAGTAT |
| | | TTCATACTGTATCTTCCCCTATTTACGCAGGAACTGATTTGGTGTAAACTTAATGAAAAC |
| | | TATTTCCATGTTGTATTACCATCTTTACTGAATAGTAGGAACGTTCATGATAACCACAGT |
| | | ACCTATATAAATAAAGATTGGTTACTTGCCCTTTTAGAGCTAACTTCCAACCTGAACCAA |
| | | AACTTCAAATTCGAATACATGAAATTGAGATTGTATATTTTAAGAGATGATTTAATTAAT |
| | | AATGGTTTGGATCTTTTGAAAAATCTTAACTGGGTCGGTGGGAAACTGATTAAAAATGAA |
| | | GATAGAGAAGTCTTGTTGAACTCGACCGATTTAGCTACGGATTCTATTTCTCATTTATTA |
| | | GGTGATGAAAACTTTGTTATTTTAGAGTTTGAATGCTAA |
| YJR101W | RSM26 | ATGCTTGTTTTCAAAAGGGGTATTCACGTAGTACCTAAACTACCAAATTCTAAGGCCCTA |
| | | CTGCAAAATGGAGTGCCTAATATACTCAGTTCTTCGGGATTCAAGACGGTGTGGTTCGAC |
| | | TACCAGCGATATCTATGTGATAAGCTAACGCTGGCGACAGCGGGACAGTCTTTGGAATCA |
| | | TATTATCCATTTCACATATTGCTGAAAACTGCAGGAAATCCATTACAGTCAAACATCTTC |
| | | AATTTGGCGTCCTCTATCCATAATAACCATTTATTCGTGGAGAACATTCTACCTTCTGCT |
| | | GTTGAACATGGCACTAACTCGAACGCCGTTGTGAAGACAGAGCCTTCTAGGCTTTTTTTA |
| | | TCCAAAATCAAAGATTCATTCAACGGCAGCGATTGGGAAGTAGTGAAGGAGGAAATGATT |
| | | TACCGTGCTGAGAATGAAGTCTTGGGACAAGGCTGGTTATTTCTAGTTGAAAACAATGAG |
| | | AAAAAACTCTTCATATTAACGAGCAATAATAATGGTACGCCGTATTATTTTCCCAGGAAC |
| | | CAGTCATTTGATTTAAACAGTGCGATATCCATCGATGAATTTGCTACCTTGAAACAAATG |
| | | AAAGAACTGATAGGAAAGTCTACTAAGTTGAATGGTAAGGTTCAAGACTGGACCATGCCT |
| | | ATAATCTGTGTCAATCTGTGGGATCATGCTTATTTACATGATTATGGCGTTGGTAACCGG |
| | | TCCAAATACGTGAAGAATGTTCTAGATAACTTGAATTGGTCAGTGGTAAATAATAGGATA |
| | | TTCTCTGGTATATCGAAGTAA |
| YPR166C* | MRP2 | ATGGGTAATTTCAGATTTCCTATAAAAACCAAGCTACCACCAGGGTTTATCAATGCTCGC |
| | | ATACTTAGGGATAACTTCAAAAGACAACAATTTAAAGAGAATGAAATCCTTGTTAAATCT |
| | | TTGAAATTCATCGCTAGAAATATGAACCTTCCAACAAAACTGAGGTTGGAGGCTCAGTTA |
| | | AAACTAAATGCATTGCCAAACTACATGAGATCTACGCAGATCAAAAATAGGTGCGTGGAC |
| | | TCTGGACATGCAAGATTTGTTCTAAGTGATTTTAGGTTGTGTAGGTATCAATTTAGAGAA |
| | | AACGCTCTGAAGGGTAATTTGCCAGGTGTTAAGAAGGGTATTTGGTAA |
| YER061C* | CEM1 | ATGTCAAGAAGAGTGGTTATCACAGGATTGGGCTGTGTAACGCCGTTGGGAAGATCATTA |
| | | AGTGAGTCATGGGGGAATCTGCTCTCTTCCAAAAATGGACTCACACCAATCACATCTTTG |
| | | CCCAACTATAATGAGGACTACAAACTCAGAGAAAAAAGTATCCCATCAACGATAACAGTG |
| | | GGGAAGATTCCAGAGAATTTTCAAAACGAAAATTCAGCCATCAATAAACTGCTGTTCACT |
| | | AGCCAGGATGAGAGAAGAACCTCAAGCTTTATCAAGCTAGCACTACGTACAACTTATGAA |
| | | GCGCTTCACAATGCTGGTCTCTTGAACCCAAATGATATAACCATCAATACATCTCTGTGC |
| | | AATCTGGATCACTTTGGTTGCCTGATAGGTTCTGGTATAGGATCCATTCAAGACATATAC |
| | | CAAACTTCTCTACAATTCCATAACGACAATAAAAGAATAAATCCATATTTCGTCCCTAAA |
| | | ATCCTTACAAATATGGCAGCTGGTAATGTTTCCATCAAGTTTAACCTTAGAGGATTATCC |
| | | CATAGTGTTTCCACAGCATGCGCAACAGGTAATAACTCCATTGGCGATGCATTCAATTTT |
| | | ATTCGGTTAGGCATGCAAGACATCTGTGTCGCCGGTGCAAGTGAAACGAGTTTGCATCCG |
| | | TTAAGTTTAGCAGGTTTCATCAGAGCAAAGTCGATTACTACAAACGGGATCTCTAGACCC |
| | | TTTGATACACAACGTTCTGGATTCGTACTTGGTGAAGGATGCGGAATGATTGTCATGGAA |
| | | TCGCTAGAACATGCTCAAAAGAGAAATGCAAACATAATTTCTGAGCTCGTGGGCTATGGT |
| | | TTAAGCAGTGATGCCTGCCATATTACCTCCCCTCCTGCTGATGGAAATGGTGCCAAAAGA |
| | | GCAATAGAGATGGCTCTAAAAATGGCTAGATTAGAACCAACTGATGTTGACTACGTCAAT |
| | | GCACATGCTACATCAACTTTACTAGGCGATAAAGCAGAGTGTCTGGCAGTAGCCTCAGCA |
| | | CTCTTACCAGGAAGATCCAAAAGCAAGCCACTGTACATATCCAGTAACAAAGGTGCAATT |
| | | GGCCATCTTTTAGGTGCACGTGGCGCCGTAGAAAGTATATTTACAATTTGTTCCTTGAAG |
| | | GATGATAAGATGCCGCATACCTTAAACCTGGACAATGTTCTGACTCTAGAAAATAACGAG |
| | | GCCGATAAGCTACATTTCATAAGAGACAAACCTATTGTGGGAGCTAATCCGAAGTACGCA |
| | | TTATGCAACAGCTTCGGATTTGGAGGAGTTAACACATCTCTTCTCTTCAAGAAATGGGAA |
| | | GGGAGTTAA |
| YGL218W | — | ATGGTACTGGTGCTCTCGCCGCGATTGGTGAACCAGTTCTGTGACGTGTTGAAGATCAAA |
| | | GAAGGAAGAACATCCTTGAACACTACATGCATGGACTCTTTTAGCCTTCTCTCAATGGTG |
| | | CTTTGGAGAATTTTTACAGAACAATCAAACTTAAAATCCAAATCAACGTCATTAAAGGAT |
| | | ATCCCAATTCCAGGGTTCTTTACAAAAATATTCGTGATGGCTTCTAGCTCGATGGAACTG |
| | | AACGTCATAGTTATAGGAATTGTGAACGATCCATTATTGATGAGCTGGGGCATGGTAAAG |
| | | CTGGGTGTATCCTGTTCGTTAATCAATAAAAGATTCGACTCTATGACTGTCTGTATCCGC |
| | | AACATAGCATCCTTACATGAAATCTTGCAAATCCCCTTGGCCAGAGACTTGGGCTGCGTG |
| | | ATAATGTCTAGGTCCAGAATTTCTAGCTGTGGTATTGTAGGAAAATCCACCTTCTGTACC |
| | | TTAATTCCGCTTTTCAAGATATCTAATTTCTTTTTAGACGGTGAGTTCAAAGCCGTAGAT |
| | | AACTTTTCCCGTATTCGTTCATTGAAAGAGTTATCACCAAACACAGCTTCGTTGAATCTA |
| | | AATGACATGACGATGCTGAAGTTTCTTGCAGTTGTGTTTTTTTTGTTATAA |
| YER251W | MRPS5 | ATGTTCAAGAGGCAATTATCGACTAGTGTTCGTTATCTACAGCATTATGATGAGTCGCTA |
| | | TTATCAAGGTACTATCCTGAAAGTCTTTTAAAATCCATAAAACTTGCTCAACAAACGATA |
| | | CCAGAAGATACTAAATTCAGAGTATCTCGTAACGTGGAATTCGCACCACCATATTTGGAT |
| | | GATTTTACCAAAATACATCCTTTTTGGGATTACAAACCAGGCATGCCCCATCTCCACGCT |
| | | CAAGAGGAGAATAATAACTTCAGCATCTTTAGATGGGACCAGGTACAACAACCATTACCA |
| | | GGCGAAGGTAACATTCTGCCTCCAGGAGTCAGCTTACCAAACGATGGTGGTCGGAAATCG |
| | | AAAAGCGCCGATGTAGCTGCAGGGCTACACAAGCAAACCGGTGTAGATCCGGATTATATC |
| | | ACTAGAAAGTTGACTATGAAGCCGCTGGTGATGAAAAGAGTGTCAAATCAGACTGGGAAG |
| | | GGTAAAATTGCGTCTTTCTATGCCTTGGTTGTCGTTGGTGACAAAAACGGTATGGTAGGT |
| | | TTGGGAGAAGGTAAATCTCGTGAAGAAATGTCCAAAGCGATCTTTAAAGCTCATTGGGAT |
| | | GCGGTAAGGAACCTGAAGGAAATACCTAGGTATGAAAACAGAACTATTTATGGTGATATA |
| | | GATTTTAGGTATCATGGTGTGAAACTACATTTAAGAAGTGCAAAACCAGGGTTTGGATTA |
| | | CGTGTAAACCATGTAATCTTTGAGATTTGTGAATGTGCAGGTATCAAAGATCTGAGTGGG |
| | | AAAGTATATAAATCCAGAAACGATATGAATATAGCTAAAGGTACCATCGAGGCTTTCACG |
| | | AAAGCTCAAAAGACATTGGATGAGGTTGCCCTGGGTAGAGGCAAAAAGCTTGTTGATGTC |
| | | AGGAAAGTTTACTATTCAAGCTGA |
| YBR282W | MRPL27 | ATGAAAGGCTCACCCATTTCTCAATTTAGTAAGACCTCTATTAATGCTCTTACCAGGCCT |
| | | TGGAAGAAATACAGAGATGGTGAATTATTTTATGGGTTATCAAAAGTTGGGAATAAAAGA |
| | | GTGCCGTTGACCACGAAGCAAGGTAACAAGACGATGTACAAAGGGACAAGAGCTTCAGGG |
| | | ATTGGTAGACATACAAAATTCGGTGGTTATGTGATAAACTGGAAGAAAGTCAGAACCTAT |
| | | GTTACCCCAGATATGGTTAATTTCGAATTAAAGCCCTACGTTAACGCAAATGTACCACCT |
| | | CTAAAACACGAATTTAAAGGATTTAGCGGCGGCCCATTAGATCCTCGTTTACAGTTATTA |
| | | AAGATAAAAGAATACATAGTAAACGGGAGGGTACAAAGCGAAGGGGCCACCGACACTTCA |
| | | TGTTATAAGGAGCGTGGATAA |
| YJL102W | MEF2 | ATGTGGAAATGGAATGTGCGCAGATGGGCGGGCGCAAGAGTAAACATTAGCAAGAACCGA |
| | | TTAAGTGTAATTAACGTTGGAAGTAGATACTTATCGACCGCAAGAAGTCCACTGTCTAAG |
| | | GTTAGAAATATAGGAATTATAGCCCATATCGATGCAGGTAAAACTACCACAACAGAGAGG |
| | | ATGCTTTATTATGCAGGAATCTCAAAGCATATTGGAGACGTCGACACTGGCGATACGATA |
| | | ACTGATTTTTTAGAGCAGGAGAGATCCCGAGGTATCACAATTCAAAGCGCTGCGATTTCA |
| | | TTCCCATGGCGAAATACTTTTGCAATAAATCTTATTGATACGCCGGGACATATAGATTTT |
| | | ACTTTTGAAGTGATTAGGGCTTTGAAAGTTATTGATTCATGCGTGGTCATTTTGGACGCT |
| | | GTTGCCGGCGTAGAGGCACAGACAGAGAAGGTTTGGAAGCAAAGTAAATCGAAGCCCAAA |
| | | ATCTGCTTTATCAACAAAATGGATCGTATGGGGGCGAGTTTCAACCATACAGTCAATGAC |
| | | TTGATAAATAAATTTATGAGAGGAACAACTACCAAACCGGTGTTGGTAAACATTCCTTAC |
| | | TATCGAAAACAACCGACTAGCAATGATTATGTTTTCCAAGGTGTTATTGACGTCGTAAAT |
| | | GGGAAGCGATTAACCTGGAATCCAGAAAATCCTGATGAAATTATCGTGGATGAGTTGGAT |
| | | GGTACTTCTCTTGAGCAGTGTAATCGTTGTAGGGAATCGATGATAGAGACTTTGACTGAA |
| | | TACGACGAAGATTTAGTCCAGCACTTCTTAGAGGAGGCAGAAGGCGATTACTCCAAGGTT |
| | | TCCGCACAGTTTTTGAACGCTTCAATAAGAAAACTGACTATGAAGAATATGATTGTACCT |
| | | GTCTTATGCGGTGCGTCTTTTAAAAATATTGGCGTGCAGCCTCTATTAGATGCTATAGTT |
| | | AATTATCTCCCTTCGCCCATTGAAGCCGAACTACCAGAACTAAATGATAAAACCGTTCCA |
| | | ATGAAATATGACCCCAAAGTTGGATGTTTAGTAAACAACAACAAAAATCTTTGCATTGCT |
| | | CTTGCATTCAAAGTGATTACGGATCCAATCAGGGGTAAACAAATATTCATTAGAATTTAT |
| | | TCAGGTACACTAAACAGTGGCAATACCGTTTATAATTCTACTACTGGGGAAAAATTTAAA |
| | | TTGGGTAAATTACTGATACCCCATGCGGGAACATCACAACCTGTAAATATTTTAACTGCG |
| | | GGCCAAATCGGATTGCTTACTGGCTCTACAGTCGAAAACAACATATCTACTGGAGATACA |
| | | CTAATAACGCATTCGTCGAAGAAGGATGGGTTGAAATCACTTGATAAAAAAAAGGAGTTG |
| | | ACGTTAAAAATCAATTCTATCTTCATTCCCCCACCAGTTTTTGGTGTTTCCATCGAACCA |
| | | AGGACTTTAAGTAATAAAAAATCGATGGAAGAAGCTTTAAACACTTTAATTACTGAAGAT |
| | | CCCAGTCTTTCAATTTCTCAAAATGATGAGACTGGCCAAACGGTTTTAAACGGTATGGGT |
| | | GAATTACACCTTGAAATTGCCAAGGATCGATTGGTTAATGACCTAAAAGCAGATGTTGAA |
| | | TTTGGTCAGTTGATGGTGTCCTACAAAGAAACAATTAATTCAGAAACAAATATAGAAACG |
| | | TATGAGAGTGATGATGGATATAGATTCTCCTTATCTCTGCTACCAAATTCCGATGCCCTT |
| | | CCTAACTGCCTTGCATATCCATTAGGAGTCAACGAGAATTTTCTGATAATGGAAAAAAAT |
| | | GGTAATTGGGATAAAGAATGGAAATACCAAGTTTCTTTCGAATCAATTCTAAATTCTATT |
| | | ATTGCAAGTTGCATTGTGGGACTTCAAAGAGGCGGAAAAATAGCTAACTTTCCCTTATAT |
| | | GCATGCTCCATCAAAATCAATAGCGATTGGTCAGTCCCACCTGATATTGAAACTCCACAA |
| | | GAAATCTTAAAAATTACTAGAAACTTAATTTTTAAAGCTCTCAATGACTTAAAACCTGAA |
| | | AAATACAACCTTCTGGAACCCATCATGAATCTTGATCTAACAATCCCTCAATCTGATGTT |
| | | GGTACGGTGTTACAAGATCTAACAGGAGCAAGAAAGGCTCAAATTCTGTCTATTGAAGAC |
| | | GAATCAAGCGTGTCAAATTCTGGCGCATCCACTTGTAATTCTCCAGAGAACAGCAATAGG |
| | | ATATATATTCCCTCTGATGCTGTTACAACCCTACACGCAACCAAAGATAAAAAAAACACT |
| | | CAAGAGACCAGTTCTAATGTAAAAAAAATTATAAAAGCGAAAGTGCCACTAAGGGAAATT |
| | | ACCACCTACACCAATAAGCTAAGGAGCTTATCGCAAGGGAGGGGTGAGTTCAATATTGAA |
| | | TATTCCGATATGGAGAAAGTTACCAACGATCGCCTACAATCAATACTTCACGACTTGTAG |
| YNL073W | MSK1 | ATGAATGTGCTGTTAAAAAGACGCAGTTTAACGTTTGCACCGAGATGGCTGTGGTGCAAG |
| | | TGTCGAAGTAGCAGGAGCAGGCCGTATTCTCTGGCACACGCGGTCGACACAAGTAAGATG |
| | | GAGGCAACCAGGAGGAACGGTCAGATTGTGAAGGACCTGGGTAGATACTATCCGTCAATG |
| | | TCAGAATCAGCACTCCACGATCTTTGCCAGGAGTATAAAGAAGTCACTATCGCAGACTTT |
| | | AATGAGAGATTCTTGGGCAACCCAGCCACATTACACCATGAGGATAATCCTAATTTGTTA |
| | | CTTAGTATTAATGGCAGGATTAAAAGCATTAGGTTTTCAGGCCAGAAGATTGTTTTTATA |
| | | GATCTTTACAACGGCAGTAGCGGATTGAAGAATGATACGCAACTGCAGTTGATCGTAAAC |
| | | TACAATAAAATTGGTGGGAGTAGTGAGGATAAGGCGAATTTCTCAGAATATATGAACTTT |
| | | TTGAAGAAAGGAGATTATATTAAGGCGCTCGGTTATCCCGGGTTTTCCCAATCCAGAGTC |
| | | AAGATGCTGTCCTTGATATGTAATAAGCTACCTATAGTATTGTCTGTTTCGCAACTGCCG |
| | | TTACCTTCCAGACTGAATGATGAAACCAAGATCAAATCTAATAGAGTTGTTGACTACCAA |
| | | TTAAACGGCACTCAGACACTGCTAGTAAGGGCGCGTATCATCAAACTTTTGAGGAAGTTT |
| | | CTCGACGACAGAAACTTCGTTGAGGTTGAAACGCCTATACTGTCATCAAAGTCCAACGGT |
| | | GCCATGGCGAAGCCCTTTATAACGTCGTCCAAGGACTTCGACCATTTGGAGTTGCGTATT |
| | | GCACCAGAATTATGGCTGAAGAGGCTTATAATTAGTGGTTTACAAAAAGTATACGAAATA |
| | | GGAAAGGTCTTTCGAAACGAAGGCATTGATTCCACGCATAATGCTGAGTTTTCCACTTTA |
| | | GAATTCTATGAAACTTACATGTCAATGGACGATATAGTCACAAGGACTGAAGATTTGTTC |
| | | AAATTTTTGATTACTAACTTGCAAAAGTTCTTTCAAGATACCCGTCTGCCTGTCCCTAAG |
| | | ACGTTTAGTGAATTACATCTGGCACTATCCGAGAATAATTGGAAGTTTAGAAAAGTCGAA |
| | | TTCCTGCCCACTTTGAACAAAGAGCTTGGCATTGATCTAATGAACTCTGGATTGGATATC |
| | | AATAAGCCGAGCGAACTTTTGAAGGCTTTACCGAAAGACATTGCGAAGAAGTATTTTCCT |
| | | TCTGCAGATAACACTGGGCAATTATCTTCGCTGCAAATCCTAAATAAACTATCGGACGTG |
| | | TTCCTCGAGCAGCGCCATTGTCAATCAACCTTGCCTACGGTAATATACCATCAACCTGCA |
| | | ATATTATCGCCCTTAGCCAAGACGGATCCGCAAAATAAGCAAGTCACGAAGAGATTCGAA |
| | | GTCTTCATCAAAGGTAAGGAATACATCAATGCTTACGAAGAGGAAAATTGCCCCCAATTA |
| | | CAACTGCAGAAGTTTCTACAACAAAAGCAGATTAACGAATTGACAGGAAACAAAACTGAG |
| | | ACCTTATCGCCTGTTATTGACTACCAGTATGTTGAGACGATGAAATACGGTATGCCTCCA |
| | | GTAGGTGGCTTTGGCCTTGGCATAGATAGATTGTGCATGCTATTTTGCGACAAGAAGAGG |
| | | ATAGAAGAGGTTCTGCCCTTTGGCTGTGTGGATGATGTAAACAGGCAGTAA |
| YOR201C* | MRM1 | ATGACTTCCCTGACTAATGCCGTGTTCAAACGATACCTGGCAGTGACTCCTAGCGCTCAC |
| | | CAAGCTCTTAAAACGAGAATTAAGAAAAAGTCGTCATCTTTCGATAAGTTTTTCCCACAG |
| | | CAAAGCAATAGTAGAAAAAAACAATGGGAAACGTTGAATGAAGACAAAGCGTCGTGGTTT |
| | | AAAAGGAAATACGCTCACGTACATGCTAGGGAACAGGACCGTGCAGCGGATCCATATGGC |
| | | AAGAAAAAGGCCCACGTCGAGAAGCTAAAGGAGATCAAGAATCAGGCGAAATTAAACCAA |
| | | AAGAGCCATAAATCCAAGTTCCAGAATAAGGACATTGCTCTGAAACTTATGAATGATAAT |
| | | CCTATCTTCGAATATGTCTACGGCACCAACAGCGTTTATGCAGCGTTGCTGAACCCGAGT |
| | | AGAAATTGCCATTCTCGGTTGCTGTACCATGGCACTATTCCTAGTAAATTTCTTCAGATA |
| | | GTCGATGAACTGAAGGTTACGACAGAGCTGGTGGACAAGCACAGATTAAACTTGTTGACC |
| | | AATTACGGGGTGCACAATAACATTGCTCTTGAGACAAAACCTTTGCAGCCTGTGGAGATT |
| | | GCGTACCTAGGCGACATGGACACCAGTTCAGCAGCATTAAGCATTCACGAGCTGGGCTTC |
| | | AATAACGAAAATATACCTCATGAACTGCCTTATGGTACGAAAACAGACGCTAAGAAGTTT |
| | | CCTCTCGGGTTATATCTGGATGAAATAACTGACCCTCACAATATCGGTGCCATTATTAGA |
| | | AGCGCTTACTTCCTGGGGGTTGACTTCATTGTGATGTCGAGAAGAAACTGCTCTCCTTTG |
| | | ACGCCTGTGGTGTCCAAGACAAGCAGTGGGGCCCTCGAACTTCTGCCCATATTTTACGTT |
| | | GATAAACCGTTAGAGTTTTTTACTAAATCACAAGAAATGGGTGGCTGGACGTTTATTACA |
| | | AGCCACTTAGCTAATGCCACGTCAGAAAAATACACTGTTGGAAAAACTATTAGCATGCAC |
| | | GATTTGAACGGATTATGTAACGAGCTGCCTGTTGTTCTAGTGGTGGGGAACGAAAGCCAG |
| | | GGCGTAAGAACGAATTTGAAGATGAGGAGTGACTTCTTCGTCGAAATTCCCTTTGGTGGC |
| | | ATTGAGAAAGGCAATCGGGCCCCAGAACCCATTGTTGACTCACTAAACGTAAGCGTGGCT |
| | | ACTGCATTGTTAATAGATAATATTCTAACTTGTAAATAA |
| YMR287C* | DSS1 | ATGGTAGTTAGGCGGAAAGTACATGTTCTTCTAATTGCAAGGAGCTTTCATTCCTACACA |
| | | CCTTGCTTCAGAGTAACTACCAGAGGCAAACGACAGCGAAGTAAGAGTAAGCAGCAAGCA |
| | | AAAGTAGAACTAGATCACACACGAGAGCTAGATAATGATCAAGCTACAGAGACAGTTGTA |
| | | GACCGTTCGGTTGGACCGGAGAAAGATATAGAATCAATAAACAAAGACTTTCTGCAACGA |
| | | ACAAAAGGATTAGAGCCTGATATTGAATTAAAACAACTGCCTCAAATTAAACAGGAATTT |
| | | AACCAACGTTATAAAGATAGGTATGTTAAACCTTCCGAAGATTGGTACGTAAACTCCTGG |
| | | AGGTCTTTAACAAAACCAAAAATTCCGCTTTACAAGTTAATAAATTCAGATTTTCAATTA |
| | | ATAACAAAACTGAAAGCTCCAAACCCAATGGAATTTCAACCTGTTCAGCTAATGGAAAGT |
| | | CCCTTAAATGTTGGTGACTTTGTTCTTTTAAAAATGAGACCCAATGAATTGGCGATGTGT |
| | | GTAAGTCTTCCAAGTAGCACTATGGATCCTCGATACACTTTTGTTACTATTGATGGAACT |
| | | ATGTGCTTTGCGACCAAAAACCGGGTGTTATTGAGGATTCCTCACAAATTACCTGCTGGA |
| | | ATCCATTCACTTATACAGCCTGAGAGCCATCACAAACATTTGCCAATTGGAACCGTTAAG |
| | | AACTTTTCTAACCAAACGAATATTTTGCCTATTGTAGCAAGGCAGCTAATAACAAGTAGG |
| | | TATCCAGCCCAAATTTCAAAGTTGGCCTGGAAAGATTTGCCCATTACTACCAAAAAATTA |
| | | CAGTTACTCCACAGATCATTACAAAATTATATGGGTCCATGGCAGATTCCTTTTTTTACC |
| | | CTAGTCGGATTAGTTCAGAAATTGGATTTAAATAAAGCCTTGGACGATAAAAATGGAATA |
| | | AATTACTTAACTAGTTTAGTTAATAACTATCACACTGTAAACGATATACCAATAAATTCA |
| | | CCTACCTTTGTTTCCACGTACTGGGCGATTATGCAACAACAGGAATCGAACCTTTGGGGG |
| | | GAAATCCATCTGAATACAGCGTTGCTGTCGCCTATATCAGTAACGATTATTCCCTTAAAA |
| | | TCACAACATTTATATTATGCGCAGGTGATAGAGAAGCTAGAAGCCAACAGTTATAGAGAG |
| | | GTCAATAAGTTTGTAAAATTGGTGAATGAAAGGAAATATCGCGATATATCAGCTTTATAT |
| | | CCTTCTGTGATCCAATTATTGAAAGATTTTGCTGCTGGGAATTTTCACAATAACGGAATT |
| | | ATAGTAGCTTTGATCTCAAAAATATTCAGAAAGATAGAACGCTATAAGGATTGTGACATC |
| | | ACAAGAGATATATGTCAAGATTTAATCAATGAGATAACACCCAACTCAATACCCAATCCA |
| | | TTGTTACTTAACATGGATTTAGCATTACCTGCTTCCTCGAAGTTAGTGAAATGGCAACAA |
| | | AAACTCTACGACTTAACTAATATAGAAGAATTGCAATGGAAAAAATCCGGCACTGATGAC |
| | | GATAGGTACGATTTTGGCGATCTTAGGGTTTTTTGTATAGACTCTGAAACTGCGCATGAG |
| | | ATTGATGACGGCGTGTCGGTGAAAAACTATGGTAGAGATGGACTGTACACTTTATATATT |
| | | CATATTGCGGATCCAACTTCTATGTTTCCGGAAAGCACTAATGTTGATATTGAGGGTATA |
| | | AGTACAGATATCCTAAATGTTGCCTTGAAAAGATCATTTACCACATATTTACCAGATACG |
| | | GTTGTTCCTATGTTACCTCAATCTATTTGTCACTTATCAGATTTGGGGAAACAAGGACAA |
| | | AGGACGAAAACTATATCCTTCTCTGTTGATGTTAAAATCACTTCTAAATGCAGTGGAAAA |
| | | TCAATTGAAATTATGTATGACAGTTTTAAAATCCGGAAAGGCATTGTGTCCAACTTCCCA |
| | | AAGGCCACTTATGAAGATGTTGACAGGATACTCGGTACACCCAATAGCGAAGCATCACCT |
| | | GTCAAAAAGGATTTGGAAAGTTTGTCCATGATTTCCAAATTATTGAGAGAACAACGTATA |
| | | AAAAACAGTAATGCTGTTATTTTCGGGGAGGGCTTTAATAAGGGCCTAGTGATGCTAAAC |
| | | GCCGATTCAGAAGGAGAACTGACTGAGGTAACATTTTCGGATCAAGAGGAGACGCTGTCT |
| | | ACCATTTTGGTTTCAGAAATGATGATCTTGGCGAATACTTTAACGGGTAGATATTTTGCG |
| | | GAGAACAAAATCGGTGGTGTGTTCAGATGTTATAAACAGTTACCACTGGGCGAAGTAGCA |
| | | CAACAACAGTATGATTCTATGATAACAAGTACAAAAAAAGGAATTTTTCCCAAGCTAAAA |
| | | GATATCGTTAAGCTTTCTTCATTGTTGAACTCGAGTTTTTATACAGGTCGACCTTTTAGA |
| | | CATGAAATGATTGGAGCTAAACAATATTTGACAGTAACATCCCCTCTTCGTCGTTTTCCA |
| | | GACTTGATAAATCATCTACAAATTCACCGTCATTTGCAGAAAAAGCCTCTATGTTTTAAT |
| | | CAAACACAAATCGACAGTTTGATCTGGCCAATTCAATCTCGAGCGGATATTTTAAAAAGG |
| | | GCTAGCCGTAATTCGTCGACGTACTGGACACTGAATTATTTAAAAAAGCTAACCAAACTA |
| | | GAGCCAGAAAGGACTTTCGATGTAATGGTAACATCAGTACCGCAGAACGGATTTACTGGA |
| | | TGTGTTTTTCCAGACTTATCGTTCGCTAGAGGAACATTAAAATTGCATCCAAGTTCTATG |
| | | CATTATCCGATGATTGGAGATATCGTGAAGAATTGTAAAATTTCAAAAATTGACTGTTTA |
| | | GAAGGAATGTTAGAGTTGGAAAAGCTATAA |
| YML110C* | COQ5 | ATGTTGATTTCTTCACGGATCGTTCGAAGCTCGCTGGTAAATGTCCCGCTAAGATTATCT |
| | | AGGTGTTTTACGCAAGCTCACAGAGCATGCAAAGAAGAAGAAGTTAATAGTCCTTTATCA |
| | | TCCGCAGCTGAACAGCCAGAGCAGAAGTATACGCATTTTGGTTCGAAGACTGTATTGAAG |
| | | TCTACCAAGCAGAAGTTAGTTGGTGATGTCTTTTCTTCCGTGGCCAATCGGTATGACTTG |
| | | ATGAATGATGTTATGTCATTAGGAATTCATAGATTGTGGAAGGACCATTTTATCAATAAA |
| | | CTAGATGCGGGAAAAAGGCCAAACTCTACGACTCCTTTGAACTTCATAGATGTGGCTGGG |
| | | GGATCCGGTGATATTGCTTTCGGATTACTAGACCATGCTGAGTCGAAATTTGGTGACACT |
| | | GAGTCTACAATGGATATTGTAGATATCAACCCTGACATGCTTAAAGAAGGTGAGAAGAGA |
| | | GCCATGGAACAAGGAAAATATTTCAAGGATCCTCGTGTGAGATTTTTGGTTTCTAATGGT |
| | | GAGAAACTAGAGGAGATTGATTCTGATTCCAAGGACATCTACACAGTCTCCTTCGGTATC |
| | | AGAAATTTCACCGATATTCAAAAGGGTTTAAACACTGCTTATAGAGTTTTGAAACCGGGC |
| | | GGTATTTTTTATTGTCTAGAATTTTCCAAAATTGAGAATCCCCTAATGGACTTTGCTTAC |
| | | CAACAGTGGGCTAAGGTCCTACCTGTAATGGGCTCGATGATTGCTAATGACTACGACTCT |
| | | TACCAGTATTTGGTGGAGTCTATCGAAAGATTTCCTGACCAAGAAACGTTCAAATCCATG |
| | | ATTGAGAAGGCAGGATTCAAATCTGCTGGCTACGAAAGTTTAACTTTTGGTATATGTGCC |
| | | ATCCATTGGGGCATTAAAGTTTAA |
| YLR439W | MRPL4 | ATGTGGAAGAGATCGTTCCACTCCCAAGGAGGGCCCCTGCGTGCCAGAACAAAGTTTACC |
| | | AAACCAAAGCCAAAACAACCGGTGCTTCCCAAAGACAAAATAAGACCGCCCACTCAATTG |
| | | ACTCATCACTCCAACAACCTGAGGATTACCGAACCAATCCCACCAACAACCTCAAACCTG |
| | | CGCTGTCCCGACGACCACCCGCTGTGGCAGTTCTTCTCCAACAAGAAATTCATCAGGTCC |
| | | GCCGACGACTTGCCGCCTTCCAGCCATATCAGACCCTGGAGCATCCCAGAGTTGAGGCAC |
| | | AAGTCCTTTAACGACTTGCATTCCCTTTGGTACAACTGCCTCAGGGAGCAAAATGTGCTT |
| | | GCTCGCGAAAACCATCTCTTGAAAAACATTGTTGGCTCCACGCATGACGAATTCAGTGAG |
| | | CTCTCTAACTCTATCAGAACCACCATGTGGCAAATAAGACATGTACTTAACGAAAGAGAG |
| | | CTGGCCTATAGCGCTTCACGCGAATTTTTACAGGATGAATCGGAGAGGAAAAAGTTCTTG |
| | | GATACTTTGGCCAACGATTACTTCTTGAACAAGGATATCCCTGATGACGAAGTCGCCTCC |
| | | ATGCTCACACGATTCCAGTTGGCCATTTTCGGCATATCGGAAACCATCCAGGATAATACA |
| | | GTCGACATTAACTTCATCGACGGGATCAAGTTCTTGGCAAACTTAAAACTACAGCGGTTC |
| | | AAGGACTCCAATGACCTCATATCCGAAATATCACAAGAACCGATCACAGACGTGGGCGAG |
| | | TCGTTCATCTTATTCACTTCCGACTTCGAACCTCATGCCGTTCAAGAGGCTTGTGTCGCC |
| | | ATCAAAGACCTGAGAAAGTCCCCAGATAATAAAGTCCCTAAATTAGACGAACTCCCCACC |
| | | GTGAGAAAATACTTGAAACAGCTAATTCATGCAAGCTCCGTGGAGCAAGCAACTGCATGA |
| YNL081C* | SWS2 | ATGGTCGTTCACATCTTGGGTAAAGGTTTCAAAGGTAAAGAGGTCATTAAAATCGCACTG |
| | | GCCTCTAAATTTTACGGCATAGGTAAAACAACAGCCGAAAAGATATGCTCGAAGTTAGGG |
| | | TTCTACCCTTGGATGAGGATGCACCAACTATCAGAACCCCAAATCATGAGTATAGCTAGT |
| | | GAACTGTCGACAATGACGATTGAAGGAGACGCTAGAGCAATCGTAAAAGACAATATAGCT |
| | | CTAAAAAGAAAAATTGGGAGTTATAGCGGTATGAGACATACATTGCATCTGCCAGTTAGG |
| | | GGCCAGCATACAAGAAACAACGCGAAAACTGCAAGGAAGCTTAATAAAATCGATAGACGT |
| | | GGAATTCACACATTTTCTCAAGCAAAAGTACAACATAATCCGTCCCTTTGGTCTTGTATC |
| | | TTTGGCAAGTGA |
| YER050C* | RSM18 | ATGCAACCTATCATCAAGGGAGCAGTAAGCTCCACATTTAAACGTGCTCTATACAATTTC |
| | | GGTATCAAAGAAAAGAAGAGTGTCAATATTGAAATGGGGAGAACGCAACAAACAAAGAAA |
| | | ATCGATCAGTCTTTGTCCAAAAAACTTCCCAAAGGTACTATCTACGATCCATTTGACTTC |
| | | TCTATGGGACGAATCCATTTAGATAGAAAATATCAGGCAAACAAGAACTCAAACAGGAAC |
| | | GATATAATGAAGTCCGGTGCAAATCCACTAGAATTTTATGCAAGACCAAGAATACTTTCA |
| | | AGATATGTGACCTCCACGGGAAGAATCCAACATAGGGATATCACAGGCTTATCGGCAAAG |
| | | AATCAAAGAAGACTATCGAAAGCCATTAGACGGTGCCAGGCAATAGGATTAATGTGA |
| YKL134C* | OCT1 | ATGCTTCGCACGATAATATTGAAAGCCGGGTCCAATGCCTCCATACCGTCACCTTCCCGC |
| | | CAAAATAAGTTACTCAGGTTCTTCGCCACAGCCGGCGCAGTCTCTAGGACCAGTCCAGGA |
| | | AGCATTAAGAAGATTTTCGACGACAATTCATATTGGAGAAATATTAATGGTCAGGATGCT |
| | | AATAATAGCAAGATCTCACAATATCTTTTTAAAAAAAATAAAACCGGACTTTTCAAGAAC |
| | | CCTTATTTGACTTCTCCAGATGGTTTGCGTAAGTTTAGCCAGGTTTCTTTGCAGCAAGCA |
| | | CAAGAACTTCTCGACAAAATGAGGAATGATTTTAGCGAGAGTGGTAAATTAACCTATATT |
| | | ATGAACCTGGACAGATTAAGCGATACGCTATGTCGAGTTATTGATTTGTGCGAGTTTATT |
| | | AGGTCAACACATCCAGATGATGCATTTGTTAGGGCAGCACAAGATTGCCATGAACAAATG |
| | | TTTGAATTCATGAATGTCTTGAACACTGATGTTTCCTTATGTAACATACTAAAGTCGGTT |
| | | TTAAACAATCCAGAAGTGTCTTCGAAGTTAAGCGCAGAAGAACTTAAAGTTGGTAAAATA |
| | | TTATTGGATGATTTTGAAAAGTCAGGAATCTATATGAATCCAGATGTTAGAGAAAAGTTT |
| | | ATCCAGTTATCTCAGGAAATCAGTTTAGTAGGTCAAGAATTCATCAACCATACAGACTAT |
| | | CCTGGTTCAAATTCTGTGAAGATACCATGTAAAGATCTGGATAATAGTAAAGTGAGTACA |
| | | TTTCTATTGAAGCAATTAAATAAAGATGTAAAAGGGCAAAATTATAAAGTACCTACATTT |
| | | GGGTATGCAGCTTATGCATTATTAAAAAGTTGTGAAAATGAGATGGTAAGAAAAAAGTTG |
| | | TGGACCGCTCTTCACAGTTGTTCTGACAAACAGGTTAAAAGATTGAGTCACTTAATCAAA |
| | | CTAAGGGCAATCTTGGCTAATTTAATGCACAAAACAAGTTACGCAGAGTATCAATTGGAA |
| | | GGTAAATGGCAAGATCGAAGATGTCAAGATTTTATTTTGACGTTAATGAACAATACTATA |
| | | GAGAAGACAGCAAATGAATTGAAATTTATAGCTGAACTCAAGGCCAAAGATCTTAAGAAG |
| | | CCGTTGACTACAAACACGGACGAAATATTGAAGCTCGTGAGACCATGGGATAGGGATTAC |
| | | TATACTGGCAAATATTTCCAGCTCAACCCGTCAAACTCTCCCAATGCCAAAGAAATAAGC |
| | | TATTATTTTACATTAGGAAATGTCATTCAGGGCTTGTCAGATTTGTTTCAGCAAATATAT |
| | | GGTATTAGATTAGAGCCAGCAATTACTGATGAGGGAGAAACATGGTCCCCAGACGTGAGA |
| | | AGATTGAATGTGATATCTGAAGAGGAAGGAATCATCGGCATAATTTATTGTGATTTATTC |
| | | GAACGAAATGGCAAGACTTCAAATCCGGCTCATTTCACAGTTTGTTGCTCTAGGCAAATA |
| | | TATCCCAGTGAAACTGATTTCTCAACCATCCAAGTCGGTGAGAATCCAGACGGTACCTAC |
| | | TTTCAATTACCTGTTATTTCTTTGGTGTGTAATTTTTCTCCAATACTAATCGCTTCTAAA |
| | | AAAAGTCTTTGTTTTTTGCAGCTTAGTGAAGTTGAAACGCTCTTCCATGAAATGGGACAT |
| | | GCAATGCACTCAATGTTAGGGAGAACTCATATGCAAAACATAAGTGGTACAAGATGTGCT |
| | | ACTGATTTTGTAGAGTTACCAAGTATCCTGATGGAGCACTTCGCTAAGGATATACGAATT |
| | | CTGACAAAGATTGGCAAACATTACGGGACTGGAGAAACAATTCAGGCTGATATGTTACAG |
| | | CGCTTCATGAAAAGCACTAACTTCCTTCAAAATTGTGAAACATACTCTCAAGCAAAGATG |
| | | GCTATGCTGGATCAATCATTTCATGATGAAAAAATCATTTCTGATATTGATAACTTTGAC |
| | | GTTGTGGAAAACTATCAAGCACTAGAACGACGTTTAAAGGTCCTAGTGGACGATCAGAGT |
| | | AATTGGTGTGGAAGATTCGGCCATTTATTTGGATCGGGGCAACTTATTACAGCTACTTAT |
| | | TTTGATAGGACGATAGCTTCTAAAATCTGGTACGCCCTTTTCGAGGATGATCCGTACAGT |
| | | CGAAAAAATGGTGATAAATTTAAAAAGCATTTACTAAAATGGGGAGGGCTAAAAGACCCT |
| | | TGGAAATGTATCGCTGATGTTTTAGAATGCCCCATGCTAGAGAAAGGCGGTAGCGATGCG |
| | | ATGGAATTTATAGCACAGTCTCACAAGTCTTAG |
| YER087W | AIM10 | ATGTTGAAATATCGTACATTGTCTCGATCATGTCATATTTTTCACCCAAAATCTCTTTCA |
| | | AACAATACCCTGAAATCTGAAACTACTCAGGAACTACTTCAAACTGTGGGATTCGTAAGA |
| | | CGCTCGCAGGTTGGGTTGTTCCAATGGCTGCCACTTGGACTCAGATCACTTAATAAAGTA |
| | | TCAAATGCTATACGGAACCGAATGGATAGCGATGGTGGAGCCATAGAAGTTTCATTAAGT |
| | | GCTATCTCGTCAAAAGCATTATGGCAAGCGACGGATCGCTGGAACAACAGTGAATTATTT |
| | | AAATTGAAGGATTCTAAAGGGAAGCAGTACTGCTTGACAGCAACATGTGAGGAGGACATT |
| | | ACAGATTTAATGAAAAATTACATTGCTAGTTATAAAGATATGCCTATCACAATATACCAA |
| | | ATGACAAGAAAATATAGGGACGAAATCAGACCCAGGGGTGGTATCTTACGTGGACGAGAA |
| | | TTTTTAATGAAGGATGCGTATTCGTTTGCTAGTAATGAAGAGGATGCGTTTGCCAGTTTT |
| | | CAAAAGCTAGATGATACTTACAATAAGATATTCAAAGATCTGAAAATCCCCTTTGTTAGT |
| | | GCGTGGGCAGATAGCGGTGACATTGGCGGTGAGTTTAGCAAAGAATTTCACCTGATTCAT |
| | | GAATCCGGTGAGGATACCTTGATGAGTTGCAAACATTGCGGCGATATATCAACTTTGGAT |
| | | ATGTCCCAGTCATATCCTGAAAAAGATGGTCAATATTCAGGTGATGTCGATTGTAAGTAT |
| | | GCTTTGACTAAGGATCACTCAACATTGATATGTTTCTACTATCCAAAGGATAGACAATTG |
| | | AATTGGAATTTAGCATTAAATGCTATGGATAAAGACATCGATTTGACGTTGAGGAACAAA |
| | | CCAAATGATCACGTCTTACAAGTCTACGAAAAAGATAACGAAGATATTATGTTCAGTAAA |
| | | ATATTAAGAGTTATGGACTGTAGATTGAATTCAAAATCCAACTTTCCTGATTTTCCACTA |
| | | AAGAAATACCTGAAAAATAATTTTGGTCAAATCAGTGACGTTTCTATAGTGGATGCACAG |
| | | GAAAACGAAATCTGTGGCAAATGTGAGGAAGGAAGGTTAGAACCTCTCAAAAGTATTGAG |
| | | GTAGGCCACATATTTTTGCTGGGTAATAAATACTCGAAGCCGTTAAATGTCAAGTTTGTC |
| | | GACAAAGAAAACAAAAACGAGACATTTGTCCATATGGGTTGCTACGGCATTGGAGTAAGC |
| | | AGGCTAGTTGGAGCAATTGCTGAACTAGGGCGTGATTCCAATGGATTTAGATGGCCCGCT |
| | | ATAATGGCTCCATACAAAGTATCGATTTGTACTGGACCAAATAATCCTGAAAACTCACAG |
| | | AGGTTACAGGATGTTAAATCAGAGCTGCTGAATGATCCTACCATGCAAAATTTACAGAAT |
| | | GACATCCTTGACCAGTTTAATGAAAAACTGGGTATAGGTGCTAGAATAAAATTATCGCAT |
| | | GCCATGGGTATACCATTATGTGTTATCGTGGGCTCAAAAAGTTGGCCCAATGTGGAAATT |
| | | GAAGTTCGTGGCATAAGATGGGGAGAAAAGGACCTGTGGAGAAAGCAGTTTGAAAAAAGA |
| | | TGTAGCGAACTGCAATGGAAGTGTACCAAAAATGAACATGGAATTGAGAAACATACTGTT |
| | | CCAATCCAGCATTTAGCGGAAGTCATTGGCGTATTGCTAAAGGATATGTAG |
| YJL193W | — | ATGTTTCAACAGCTGTCGGCATCCATTAGGCACAATGCACACATAATTTTTTTATGCATA |
| | | AGTTGGTATTTTATTTCATCGTTGGCATCTCAGGTAACGAAGCAGGTACTAACGGTTTGC |
| | | CCATTACCACTTTTTTTGGGTGAGTTTCAGTTCATTTATACTGCTGTGCTAGCATGGTTT |
| | | ACGTGTTATATTGCATATAGTTTCCCAGGATTCTATCGCATATTTCCTAATGGTACTTTT |
| | | CCGGAATATTATATCGACGACAGAGAAACTAGCCGTGCAGCTAGGAAAGAATCAAAACTA |
| | | TCCTCCCTCATTATACCGCCCTCTAAACCAATCCTGCAAACAGTTCTACCTTTAGGTCTT |
| | | TTCCAATTTGTAGGGAAATATTTTGGGCATACAGCTACCTCTTTGGTGCCTGTTTCCACG |
| | | GTTGCAAGTATAAAAACGCTATCCCCCATGTTTATCCTTTTGCTCCAAAAGATTTTGAAA |
| | | ATATCCACTTTGAAAATTACTTTAACTCTTATCTTTAGTTTATGCACTTTAGTTCTTGGA |
| | | GTTTGGATTATAGTTCAAGAAGATAATCGTTCACCGGCCTCTTCCAATGAGCTGAGAGAA |
| | | TTCTCTAAATACGGCGTAATATGCGCTATGATATCAATGTTCATTTTTGTCTTGCAAAAT |
| | | ATATATGGCAAGACCGTGTTCACGTACAGATCTCAAACAGATGAATCTCAAAGTAATTCT |
| | | GGATTCTCACGGCAAGAATCACCTTTACCCTTATATGAAAAGTTGGATGAGAAACTTGTT |
| | | GCGAAAAAGAAACCTAAATCATATGACAAATTGACTTTAATGATTTATATTTCCCTTGTT |
| | | GGATTTTGTTTATCGTTTGGCTGGTTTATCACTCTAGAATTTCCTGTACTATTTAGGTAT |
| | | TTTTTTCAAATTAACAGCTCTTCTACAGTAATAAAAGCTTTTCCAGTCTCTTTATTTTTA |
| | | CTGAACGGGACATTCCACTTCATTCAGGCAATGATTACATTTCATTTACTGGGTGAGGTC |
| | | TCAACTTTAACATATTCAATAGCGAATCTGATGAAGCGCTTTGCCATTATTGCGGTTAGT |
| | | TGGGTCTTTATTGGAAGAAGAATTACTTGGCTGCAAGTGTTCGGTTTGGTTCTGAACACA |
| | | TTAGGGTTATTCCTTTATGAAAGATGCACATCACAATCGAAAATAAAAGCGAAAATACGT |
| | | CCTGAGTAG |
| YLL006W | MMM1 | ATGACTGATAGTGAGAATGAATCCACCGAAACGGATTCGTTAATGACGTTTGACGATTAT |
| | | ATAAGCAAAGAGCTACCTGAACATTTACAGAGACTAATCATGGAGAATTTGAAGGGTTCT |
| | | ACTACTAATGACTTAAAGCAAACTTCAAACAACTCAGAGTTTAATGTCAGTAAAAACGGG |
| | | AGCTTCAAAGGTCTCGATGATGCAATTCAAGCTTTGCAAATGCAAAGCGTGTTGCATCCT |
| | | TCTTCGTTAGGATCGTTAGCAACGTCCTCCAAATTTTCTGGATGGTCGTTTGCTCAAGGG |
| | | TTTTTTGTAGGACAGCTAAGCATAGTGTTGTTGTTCATCTTTTTCCTAAAGTTCTTTATA |
| | | TTCAGTGATGAGCCATCTAAAAGTAAGAATCCGAAACCTGCAGCCTCCCGTCACAGATCA |
| | | AAATTTAAAGAATATCCCTTTATATCTCGCGAATTCCTGACTTCTCTTGTTAGGAAGGGT |
| | | GCTAAACAACACTACGAGCTCAATGAAGAGGCAGAAAATGAACATCTTCAAGAACTAGCT |
| | | CTTATTTTAGAGAAAACCTATTATAATGTCGACGTGCACCCTGCAGAGTCATTGGACTGG |
| | | TTCAACGTTTTAGTTGCCCAAATAATACAGCAATTCCGCAGTGAGGCTTGGCACAGGGAC |
| | | AATATCCTTCATTCCTTGAATGATTTTATTGGAAGAAAATCACCCGATCTGCCTGAATAT |
| | | TTGGATACCATAAAAATAACTGAACTGGATACAGGTGATGATTTCCCCATTTTCTCGAAT |
| | | TGCAGAATACAATATTCGCCAAATTCAGGAAATAAAAAGCTAGAGGCTAAAATTGATATA |
| | | GATTTAAATGACCACTTAACTTTAGGAGTAGAAACAAAACTATTACTTAACTATCCAAAG |
| | | CCTGGTATTGCCGCACTCCCCATAAATCTAGTAGTGTCAATTGTGAGGTTTCAGGCGTGT |
| | | TTGACCGTATCTTTAACTAATGCAGAGGAGTTTGCTTCTACTTCGAACGGTAGCAGTAGT |
| | | GAAAACGGTATGGAGGGCAATTCAGGATACTTTTTGATGTTTTCTTTTTCTCCTGAATAT |
| | | AGAATGGAATTTGAAATCAAGTCGCTAATTGGCTCACGGTCTAAACTTGAAAATATTCCC |
| | | AAGATCGGCAGTGTCATTGAATACCAAATAAAAAAATGGTTCGTTGAACGATGCGTTGAA |
| | | CCAAGATTCCAATTTGTCAGGTTACCAAGTATGTGGCCACGTAGTAAAAATACGAGAGAA |
| | | GAAAAGCCTACAGAGTTATAA |
| YBL090W | MRP21 | ATGTTGAAGAGCACGCTGAGGCTTTCAAGAATCTCTCTCAGAAGAGGTTTCACAACGATC |
| | | GACTGTTTACGCCAACAAAATTCGGATATCGATAAAATCATACTAAATCCAATCAAATTA |
| | | GCTCAGGGAAGCAACAGCGATCGTGGCCAAACCTCTAAAAGCAAAACTGATAATGCAGAT |
| | | ATTTTATCAATGGAAATTCCAGTAGATATGATGCAATCTGCTGGGAGAATAAACAAGAGG |
| | | GAGCTTCTATCCGAGGCGGAAATTGCTAGAAGTAGCGTGGAGAATGCACAAATGAGATTC |
| | | AATTCTGGAAAATCTATAATCGTGAATAAGAACAACCCTGCAGAATCATTTAAGAGATTA |
| | | AACAGGATCATGTTTGAGAACAATATTCCCGGAGATAAAAGAAGTCAACGGTTTTACATG |
| | | AAGCCGGGGAAAGTGGCTGAATTGAAGAGATCTCAAAGGCATAGGAAGGAATTCATGATG |
| | | GGCTTCAAGAGGTTGATTGAAATTGTTAAAGATGCCAAGAGGAAAGGATACTAA |
| YOR241W | MET7 | ATGCATAAAGGAAAAAAAAATTACCCTAATTTGATTACTTCGTTTCGGATGAATTTGAAA |
| | | AAAATCATATTGAACCATGACCGATTTAGCCATCCAGAAAGATGGAAAACAAACGCACTC |
| | | TTAAGGTTCACCTTCGTGTATATAAAATTTCTCTTCGACTTGATGATAATCAAAAATCCA |
| | | TTAAGGATGGTTGGAAAAACTTATCGAGACGCTGTTACTGCGCTAAACTCTTTGCAGTCC |
| | | AATTATGCCAACATCATGGCTATCCGTCAAACTGGTGATCGTAAAAACACTATGACATTA |
| | | TTGGAAATGCATGAATGGTCTCGAAGAATTGGTTACTCTGCATCGGATTTCAATAAATTA |
| | | AATATTGTTCATATCACTGGAACAAAAGGTAAAGGTTCTACTGCCGCATTTACTTCATCA |
| | | ATTCTAGGACAATATAAAGAACAATTACCACGTATCGGGCTATATACGTCTCCACATCTT |
| | | AAGTCAGTGAGAGAAAGAATAAGAATAAATGGGGAGCCAATTTCTGAAGAAAAATTTGCG |
| | | AAGTACTTCTTTGAGGTTTGGGACCGTCTAGATAGTACAACCTCTTCTTTGGATAAATTC |
| | | CCGCACATGATCCCGGGAAGTAAGCCTGGCTATTTCAAGTTCCTCACATTGCTTTCATTT |
| | | CACACTTTTATACAAGAGGACTGCAAAAGCTGTGTCTACGAAGTCGGCGTCGGAGGTGAA |
| | | TTGGATAGCACTAATATAATTGAAAAGCCAATTGTCTGCGGTGTTACACTATTAGGAATA |
| | | GACCATACTTTCATGCTTGGTGATACTATTGAAGAAATTGCTTGGAATAAGGGAGGAATT |
| | | TTCAAATCTGGAGCACCAGCATTCACCGTTGAGAAACAACCTCCTCAGGGATTAACAATA |
| | | TTGAAAGAAAGAGCTGAGGAGCGCAAAACAACGCTAACAGAAGTACCCCCATTTAAACAG |
| | | TTGGAAAATGTCAAGCTTGGAATTGCTGGCGAGTTTCAAAAAAGTAACGCCTCCCTTGCC |
| | | GTTATGCTGGCTTCTGAGATCTTGCATACGTCCAATATATTGGAGGAGAAGATCAAATGC |
| | | AGTTCGAATGCATCGATTCCAGAAAAATTCATAATCGGTCTACAAAATACTAAGTGGGAA |
| | | GGCAGATGTCAAGTACTAGAAAAGGGAAAAAACGTTTGGTACATTGATGGTGCTCACACC |
| | | AAAGATAGTATGGTAGCTGCATCAACATGGTTCAGAGATATGGTTCGATTGTCGAAGAGA |
| | | AAGAAGATCTTGCTCTTCAACCAACAAAGCAGAGATGCCAACGCTCTTGTGAATAACCTG |
| | | TACTCATCTGTTTCGCCAGAAATAACGTTCGATGATGTGATATTTACTACTAATGTCACT |
| | | TGGAAATCAGGCTCATACAGCGCTGACCTCGTTTCCATGAACACTTCTCAAGAAGATGTA |
| | | GAAAAATTGAAAGTCCAGGAGTCATTAGTCAAGAATTGGAACAAAATAGACGATAATCGT |
| | | GCTAAAACGCATGTAACGGCTAGTATAGAGGAAGCAAATGAATTGATTGAAACGCTATAC |
| | | GATGAACCTGCTGATATATTTGTAACTGGTTCATTACATCTTGTTGGCGGATTATTGGTC |
| | | GTTTTTGATAGAATAGATGTAAAGTAA |
| YLR295C* | ATP14 | ATGTTCCCTATCGCTTCCAGAAGAATACTGCTCAATGCTTCAGTTCTGCCATTGAGACTG |
| | | TGCAATAGAAATTTCACTACCACAAGAATATCCTACAACGTCATACAAGATTTGTATTTG |
| | | AGGGAACTAAAAGACACCAAACTGGCTCCAAGTACCTTGCAAGATGCTGAAGGTAATGTT |
| | | AAGCCTTGGAACCCACCACAAAAACCAAATCTACCAGAATTGGAACTTCAAGGCCCAGAG |
| | | GCTTTAAAGGCTTACACCGAGCAAAATGTAGAAACTGCTCATGTTGCTAAAGAGTCTGAA |
| | | GAGGGTGAGTCAGAGCCAATTGAAGAGGATTGGCTAGTTTTGGATGATGCTGAGGAAACC |
| | | AAAGAAAGTCATTGA |
| YCR024C* | SLM5 | ATGTTTCATGCTTTCACCTTCCTTAAAGGTGGTAGATTTTACTCTTCACTAACAGTTAAA |
| | | TCATTGTACGAGCAGGTACACCATACTAGCCATGATCCCATTTCAATTAATGGATGGATC |
| | | AAATCCATAAGACTATTAAAACGTATAGCGTTTTTGGATTTACAAGATGGGACTTCTGTG |
| | | AACCCATTAAGAATAGTTATTCCACTCACAAATACTGATGAAGTACAGTTCCTAAAAATT |
| | | CTGAAAACTGGTCAAACTTTATCTATATCTAATGCTACCTGGCAAAGCACCCCTAATAGA |
| | | AAACAACCTTTTGAATTGCAAATCAAAAATCCTGTCAAGTCAATTAAACTTGTGGGTCCC |
| | | GTTTCAGAAAACTATCCATTACAAAAGAAATATCAAACCTTACGTTATTTAAGGTCCTTA |
| | | CCTACACTAAAATACAGAACCGCTTACTTAAGTGCAATTTTACGGTTAAGATCCTTTGTA |
| | | GAATTCCAGTTCATGCTATATTTCCAGAAAAACCACTTCACCAAAGTTTCACCACCAATA |
| | | TTAACTTCAAACGATTGTGAAGGTGCCGGCGAGTTGTTTCAAGTCTCCACCAATACGTCG |
| | | CCAACTGCATCCTCGTACTTTGGGAAGCCGACTTATTTGACTGTGTCCACTCAATTGCAC |
| | | TTGGAAATTTTAGCGTTATCACTGTCAAGGTGTTGGACGTTATCTCCTTGCTTTAGAGCC |
| | | GAAAAGAGTGATACTCCAAGACACCTTTCGGAGTTTTGGATGCTTGAAGTGGAAATGTGC |
| | | TTTGTTAATAGCGTCAACGAGCTAACATCGTTTGTTGAGACTACAATAAAACACATAATT |
| | | AAAGCTTGTATAGATAACCAACAAGAACTCTTGCCGAAGCAATTTATCTCTTCACAAGAA |
| | | AATAATGCATCGTCAGAGCTATCAATAAATCAAGAGACACAACAAATTAAAACACGATGG |
| | | GAAGATTTAATAAATGAAAAATGGCACAATATAACGTATACCAATGCAATAGAAATTCTC |
| | | AAGAAACGCCACAATGAAGTTTCACACTTTAAGTATGAACCTAAATGGGGACAGCCTTTG |
| | | CAAACTGAACATGAAAAATTTTTAGCCGGAGAGTATTTTAAGTCCCCAGTTTTCGTTACC |
| | | GACTATCCACGTCTTTGTAAACCATTCTACATGAAACAAAATTCCACTCCTGACGATACT |
| | | GTTGGATGCTTTGATCTACTGGTTCCTGGAATGGGTGAAATAATTGGTGGGAGTTTAAGG |
| | | GAAGATGACTATGACAAGTTATGTAGAGAAATGAAAGCACGCGGGATGAATAGATCTGGA |
| | | GAATTGGACTGGTATGTTTCTCTGAGAAAAGAAGGAAGTGCACCACACGGAGGCTTTGGT |
| | | CTAGGGTTTGAGAGATTTATCTCATACTTATATGGCAACCATAATATAAAGGATGCCATA |
| | | CCCTTTTATAGAACATCTGCAGAATCCATCGATTTTTGA |
| YMR293C* | LRC6; | ATGCCACTGAAACGCTCCTTAAAAGAATCAATCGAAAGATTATCTAGTTTTCAATCAAAA |
| | HER2 | TATAATATTTTCACTAGTATCAATCCATCTCCTTATTCAATCACAAATAAAAAAGGTACA |
| | | AAAGAAACATTGACAGGTTGCGTTGCCAGTATCAAGGATAATATAGTTACAAAAGATTTT |
| | | CCAACCACATGTGCCTCACATATACTTGAGAATTTCAAGTCTCCCTTCGATGCAACAGTA |
| | | GTGAAACTATTAAAACAGGCAGGAGTGCATATCTTAGGCAAGACAAATTTAGATGAATTT |
| | | GGAATGGGTTCTGGGGGAGTACATTCAATAAGAGGGCCCGTAATTAACCCTTTATATCCT |
| | | CATGAGGACAAGAAGATCATGGGAGGTTCATCGTCTGGAGCCGCTGCAAGCGTTGCTTGC |
| | | GATCTAGTCGATTTTGCCTTGGGAACAGATACTGGTGGCTCCGTTAGGCTCCCCGCATGC |
| | | TATGGATCTGTTTTAGGATTCAAGCCGTCCTATGGACGGTTGTCAAGATTTGGCGTAATA |
| | | GCGTATTCTCAATCTTTAGACACTGTTGGCATACTTAGCAAAAAGATAAACGTCTTGCGA |
| | | AAAGTATTTCATACGCTCGATAAATATGACATGAAAGATCCCACTTCCTTAAGTGTGGAA |
| | | TTGCGTGAATTGATAGAAGGGAATAAAAAGGTTAGGAGACCTTTAAAGGTAGGAATTGTA |
| | | AAGGAATTTAGCCATGAAAGTATGCCCATTGGGTTCCACAGATTATATTTATCCTTACTA |
| | | GAAAAGCTGATAAATTTAGGGCTTGAAATATACCCTGTATCCATTCCATCTGTAAAAAAC |
| | | TGTTTACCGATTTATTATACGCTATCACCCGCTGAAGCGGCTTCCAATTTATCAAGGTAT |
| | | GATGGTATCAGATATGGATACAGAGATTCAGAACTCGATATAAAAGACGGTATTTTATTC |
| | | GCACCTACTAGGTCCAAATTCGGAACAGAAGTTAAGAATAGAATCATTCTAGGTAATTAT |
| | | AATCTTTGTTCAGATGCATTTAAGAACAATTTTATTAAGGCAGAAAAGCTACGAGTTAAT |
| | | TTGATCGATGAATTTGACGGGATCTTCAGATTTCCAAATGTTTTAACTAATTCTAAGGGA |
| | | AATCCAGACGGCCTAGATCTACTGATAGTTCCCACATCGTCCAAGCTTCCTGGATCCATA |
| | | AGGGACTTCGAAGAAGAGGAAGCCAAATCTCCGGCTAACTCGTATATAAACGATGTCTTT |
| | | ACTGTCCCAATGTCATTGGCTGGACTACCAAGTTTATCAATGCCTTTAAAAGAGAAGACC |
| | | CCGATTGGCTTACAGGTAGTAGGTCAATATGGTGATGATTCCACTGTGTTAGATTTTGTC |
| | | GAGTCAATATCATAA |
| YBR163W | DEM1 | ATGCTGGGCCGGGCACTAATCAACAAGTATGGATTTTTAATTCATCCTAGGAGATTTGTA |
| | | CATTTAAACGATAAGTCCCTTGATGGAACATTTATACTACCTTCGAAGAAAAATCATATG |
| | | TATGATGTTCCAACTAATGATCCAAGCGGTATCCTGAATGCCTCGGATATCGATAGAATC |
| | | AATAATTTGCCATTTTTCGATAACACCAGTCCGACAAAAGAGACAAATACTAAGGAAGGA |
| | | GCATTGTTGAGCGAAAAATTGGCCAGCGTTAAAGAATTATTTGGAGAAGACCCTGAAAAT |
| | | CCTTCGTTTATCAATTACAGATTCCCCAGAGGTTTAGAGAATCCCTATTTTGACATTCAA |
| | | GTAAATCAGCTAAAGAAAAAACGATTATCCGTCACACAATTATGCACTACGCAGAATTGG |
| | | TGCGAGTTAAGGAATTTTTACGACTTTTATTCTCAAAATTTGTCTAACCAACTACTGAAC |
| | | TTAAAATTTCAGGTGCAGAAAGGTAAAAAGATTCATAAATCACTAGAGGACGAAACGCAT |
| | | CCTGAATTAAATCAGTATAAAAGCTTTACCCACAATTTCCTTGCACTGACAAAATTATCT |
| | | ATGGATATTGATAATGATATGGATGCCCTGCTAGATAACTGGTTCAACTCTATCAATAGA |
| | | TTGGTCTCTTTATTCACCAAAGGTGACGGTCATGCAAGAGAAATAGTTTGTCATGGCTTC |
| | | ATTAACTTAGAAGATGGCAAACTGGTGGAGCATTTGCTGAACAGTGATAGCAAGACAAAG |
| | | GAAAATGTGATTATATCAGGTGTTATTGACCACTTAACTCTTAGAAATAGGCACAATCAT |
| | | CAAGTACAGAAAGGTGCAGCTCATCTTGATACGGAATATCAGAGCTGGGGTAATATCCTC |
| | | ACTAATTTACTATCAAATCTAAAAGAACTGAAGTCGAATAACGAGATTGTTATCTCTGAT |
| | | ATCAAAACTAGGTCAGTACCAAAAATACCATCTATTGAGTCCGTTATAGAATCCTCTAAA |
| | | TTACAGACAATGTATTATAAGTTTTTTTTTTCCCACCTTAGTCAAGACATGACACAAACC |
| | | TACCATAGCTTTTTAATCAATGCCCAGAGAAGAGGACTCGACGTTGATGCGCCCATAAAT |
| | | CCTACCAAGATTTTAACATTCATACTAACAAACCCACTCTTCGCTAATGATGTGAAGAAC |
| | | CTCCTGTACGGACTACCGATAAATCATTCTGCTTTCGATAACGACGCCAAAGGCTCTAAT |
| | | ACATTTGATATGACCGCGTTCAATGATTTGCTGGATAGAGGTCCCACATCTTTTAACGTT |
| | | CCGATTGAGCAAGACGAAGATAGTTCGGAGTCAACAAAATGTGTATCTTTGCGAGATTAT |
| | | GGACATTTTTATACGAAATGGAAGACACCTCTAACTTTGAAATACTTTGCGGCAAGGTTA |
| | | TCACAGATCTATTTTATTGTGGGTAATCTCGTTTCGAATGATTTAATGATAGAATATTAT |
| | | TACCATAACGATAATTTCCACAATATCATCTTCCCATATGATCCCTTGAAACTGGGAACC |
| | | CATGCCCATGATTCTGCAATGGTTTGGTTTGGTGGCAGAGATATGCATCCAATTGAGCCT |
| | | ACTCAAAAAAACTTCAACACATACTGTAAGTTTTGTGACTACAGACACGTTTGTTCTTGG |
| | | AAAAATAAAAATGAATTGAAACTTATTGATCTAGGGAAAGAATTAAAAAAAATTATTCTT |
| | | GAATCAAGTATGAAGTAG |
| YPL173W | MRPL40 | ATGTCTGGAAGCTATCAACATTTATCAAACGTTGGATCGCGTGTTATGAAAAGATTGGGG |
| | | AATCGACCCAAAAACTTTCTTCCTCATAGTGAAAAGTTCATTAAAAAATCTACGCCTGAG |
| | | TTCATGAAGTCAGATTTAAAGGAAGTAGATGAGAAAACTAGTTTCAAGAGTGAAAAAGAA |
| | | TGGAAGTTTATACCTGGCGACAGAGTAGTAGTAATGAGCGGGGCTTCCAAAGGAAATATA |
| | | GCTGTAATTAAATCTTTCGATAAAAGAACAAATTCATTCATATTAGATGAGAATGGGCCC |
| | | ACAAAGACTGTTCCAGTTCCTAAACAATTCTGGCTTGAAGGCCAAACATCTCACATGATA |
| | | ACAATTCCAGTTTCAATTTTGGGAAAGGATTTAAGGCTAGTTGCTGATATTGACGATGAA |
| | | AAGACCCCAGGAAAGACAAGAACGGTGGCAGTAAGGGATGTTTCCTTCAATGGTTCCTAC |
| | | TATGATGCAGACTACAAAAAGGTAATGCCATATAGATGCGTAAAGGGTCAACCAGACTTG |
| | | ATCATTCCATGGCCTAAACCAGACCCAATTGATGTGCAAACGAACTTGGCTACCGATCCT |
| | | GTAATTGCTAGAGAACAAACATTTTGGGTCGATTCCGTAGTGAGAAACCCTATTCCAAAA |
| | | AAGGCAATACCAAGTATTCGCAATCCTCATTCTAAGTACAAAAGGGGTACCTTGACAGCG |
| | | AAAGATATAGCTAAGCTAGTAGCACCTGAGATGCCCCTAACAGAGGTAAGAAAAAGCCAT |
| | | TTGGCAGAAAAAAAGGAGTTGGCTGAGAGAGAAGTACCAAAACTAACTGAAGAGGACATG |
| | | GAAGCTATTGGGGCGAGGGTTTTTGAATTTTTGGAAAAACAAAAAAGAGAATAA |
| YPL005W | AEP3 | ATGAATACATTAAGGTGTTTGACACAAGCACTATCCAAAAGTGGGAGAGAAGCTCCAAAA |
| | | CTATATCAGAAAGTAATATTTCCAGGCCTGTTCAGAGAGGGTATACCAATTGCAAATGTC |
| | | AAGAAAGTTGATGAAAAAATAATAGATAGCCCAACATCAACGTCAGTTAATGGAGAGGCA |
| | | AAAAAAATAGTACGGCATGGTGTGAAGTATGAAAGGGAGCAAGTTAAGGAATACCTTTCA |
| | | AGCCTGCCGACTTTAACGCTTTCTAGGAAACAAATTAGGGATGACTATGATGAGGAACGT |
| | | GCAAAACGAATGTATATGTTCTCTAAACAAACGAATTCCTCGAATAAATTCCAGAAACTT |
| | | CTTACAGCAAAGTCTCAAGAGTTTACTAGAGAACTATTGACGCTACTGATAGATTGCACA |
| | | TCGAATGAGAAGAATTCAGGGCCAGAACGCTTCACTCGTAAGTTTCTTAAGTTTAGTAAT |
| | | GACGAGATCCCTCCTCTCCCTGACTTTTCAAAGAACCCTCAATTGTTTGAAAATTATATT |
| | | GGAATACTAAGTCATACAAAGTTCAATTTTCGTTCATCTTCCAAACTAAATGGTATTGTG |
| | | CGTAAGATGCTGCGGCACCTACTTCATCCAACCAATAAGACGACTCTGCCGTTGCGTTCT |
| | | GCTCAAGTTTATAATGACTCAATATACTTTTTCAGTGAGCACTTCGACTTTGCGTCTTGT |
| | | AGAGAAATATTTGCACAAATGAAGGCTGAGGGGACTAAACCAAATACAATCACTTTTAAT |
| | | CTTTTACTGCGTAATGTAGTGAAAAACTCGCATATTAGAAAAACGAAGCACCCTGACGAT |
| | | GAGGTCCTTTTTTATCTTAGAAGTATGCGTAATCATGGAGTTTTTGCAGATGTAATAACT |
| | | TGGACGACATGTTATAACTTTCTTCGTGATGAAGTTTCGAGACAATTGTACATTGTTCAA |
| | | ATGGGTGAACATTTAGGAAATTTTAACGTAAATTTTGTTTATACTGTGCTAAGGAACGGA |
| | | GATTATAGAGCAGAGGACTGTTTAAAAGTTTTAGCAGCAAATTCACTACCGATTTCCCGT |
| | | AAGACGTTCTATCTGTGCATAGAACGACTACTAAACGAGGAACAACTTGAAACCGCATCT |
| | | AAATTACTTGACTATGGTTTTCAGCACTTGAAAAGTAATTTTAAGTTGGACTCGGAGGCA |
| | | ATGAATCATTTCATGCGGGTATTTGCTAATAAGGGAAGAAGTGATTTGGCATTCCTTTGC |
| | | TATAATACTTGTCGTAAGATTTACAAAATTAAACCTGATTCTCAGACGTTCGAAATGTTA |
| | | TTTAAAGCGTTAGTTAGGAATGGCAACACCAAGAATTTTGGGGCAGTCCTACAGTATATC |
| | | AAAGACTTAAAGGTTAGTGAAGGATTTGGATTGCGGACAAGTTATTGGAGAACCAAAGCA |
| | | GACTCAATTTTCAAATTTGGTAGTCCTAATACGCTATCAGAAAAGTCAATTGAAAAAGCA |
| | | AGAAAATTGTTGGGCAACCTGATTGCTTCCGAGGGTGAGTTCTCTTGGAAAATTTGGAAA |
| | | GAAAGTGATTCTTCACAAAAAAAAATACTACGTTTTTTGGGTTGTATCCCCACTACTTTA |
| | | CGCTGCACCAATACAGCACAAGATCATCAGAAGCCCACAAACCTACCCTCAAACATTTCC |
| | | CAGAAAAAAAGAGAATATCGAAATCGGGTTAAAGCAATAGCCACAAAGGCTGCTTTGGAA |
| | | AAAAGAATGGCGTATATCAAAGACAATGATGTAGCTTTCAAAAAAGAGCTAGTAAAGAGG |
| | | AGAATAGTTGGGGAGGTTTGA |
| YBR104W | YMC2 | ATGAGTGAAGAATTTCCTACGCCGCAGCTTTTGGATGAATTGGAAGATCAACAGAAAGTT |
| | | ACCACGCCGAATGAGAAGAGAGAGTTAAGTTCTAATCGTGTGTTGAAGGATATTTTTGCG |
| | | GGTACCATCGGTGGTATTGCACAAGTTCTTGTAGGGCAACCGTTTGATACAACAAAAGTT |
| | | AGGCTTCAAACAGCCACCACAAGAACAACAACGTTGGAGGTATTGCGTAACTTGGTAAAG |
| | | AATGAGGGTGTGTTTGCATTCTACAAAGGTGCGTTAACACCGTTATTGGGTGTTGGTATA |
| | | TGTGTCTCAGTACAATTTGGTGTGAATGAGGCGATGAAAAGATTTTTCCAAAATTACAAC |
| | | GCTTCTAAGAACCCGAATATGAGCTCACAGGACGTAGATCTTTCACGTTCAAATACTCTG |
| | | CCACTATCCCAGTACTACGTTTGTGGGTTGACAGGTGGTGTAGTCAATTCATTCTTGGCA |
| | | TCGCCCATTGAACAAATAAGAATTCGTTTGCAGACGCAAACATCTAATGGTGGAGACAGA |
| | | GAATTCAAAGGTCCTTGGGACTGTATTAAAAAATTGAAAGCTCAAGGTGGATTGATGAGG |
| | | GGTTTATTTCCCACAATGATAAGAGCCGGACATGGGCTTGGTACATATTTTCTTGTCTAC |
| | | GAAGCATTGGTTGCCAGAGAAATTGGTACGGGACTAACAAGAAACGAAATTCCTCCTTGG |
| | | AAATTATGCTTATTTGGTGCATTTTCCGGGACAATGTTGTGGTTGACAGTATACCCATTG |
| | | GACGTTGTGAAATCCATTATACAAAACGATGATTTACGAAAACCTAAATACAAGAATTCC |
| | | ATTTCTTACGTAGCAAAGACCATTTATGCAAAAGAAGGTATCAGAGCCTTTTTCAAAGGG |
| | | TTTGGGCCAACAATGGTTAGGTCCGCGCCCGTTAATGGTGCGACATTCCTGACGTTTGAA |
| | | TTAGTCATGAGATTTCTGGGGGAAGAGTAG |
| YER122C* | GLO3 | ATGAGTAACGATGAAGGAGAAACATTTGCCACGGAACAGACTACTCAACAAGTTTTTCAG |
| | | AAGCTAGGTAGCAACATGGAAAATCGAGTTTGTTTTGATTGTGGTAACAAGAATCCTACT |
| | | TGGACTTCGGTACCTTTTGGTGTGATGCTGTGTATTCAATGCTCTGCTGTGCATAGAAAC |
| | | ATGGGTGTTCATATCACCTTTGTAAAGTCTTCTACATTAGATAAATGGACAATTAACAAT |
| | | TTAAGAAGGTTTAAGTTGGGTGGGAACCATAAGGCTCGTGATTTTTTCTTAAAGAACAAT |
| | | GGGAAACAGCTACTAAATACAGCAAATGTTGACGCGAAAACAAAATATACGAGCCCTGTT |
| | | GCTAAGAAATATAAGATACATCTCGACAAGAAGGTTCAAAAGGATATGGAGTTATACCCA |
| | | TCAGAATTAGTCTTGAACGGTCAAGATTCTTCGGACTCACCGTTGGACACCGATTCAGAC |
| | | GCTTCCAGGTCGACTTCCAAAGAAAATAGTGTTGACGATTTTTTCTCTAATTGGCAAAAA |
| | | CCAAGCTCCAATTCCAGTTCTAAACTCAATGTAAATACAGGCTCTCTCGCCCCAAAAAAC |
| | | AACACCACAGGCAGCACACCCAAGACCACTGTCACTAAAACGAGAAGTTCCATCTTAACG |
| | | GCTAGCAGAAAGAAACCGGTATTAAACTCGCAAGATAAAAAGAAACATTCAATTTTGTCT |
| | | TCGTCAAGAAAGCCCACTAGACTAACCGCAAAGAAAGTCGACAAATCACAGGCAGAAGAC |
| | | TTGTTTGATCAATTTAAAAAAGAGGCTCAACAAGAAAAGGAGGACGAGTTCACTAATTCG |
| | | TCTTCTAGCACCAAAATTAGACAAAATGATTACGATTCCCAATTTATGAATAACAGTAAA |
| | | GGCAACAATAATAACAGTATAGATGATATTAATACCCAGCCTGATGAATTCAATGATTTT |
| | | TTAAATGACACTTCAAACAGCTTCGACACAACTAGAAAAGAACAGCAAGATACTTTAACA |
| | | CCCAAGTTTGCCAAATTGGGATTTGGTATGACCATGAATGACGCCAATGATTTGGCAAAG |
| | | CAACAAAAAGAATCTCAGAAAATAGCTCAGGGACCAAGATATACGGGCAGAATCGCGGAA |
| | | AGGTACGGCACACAAAAAGCTATATCATCTGATCAATTATTTGGTCGTGGCTCATTTGAT |
| | | GAAGCAGCAAACAGGGAAGCGCATGACAAGTTGAAAACATTTGACAATGCTACATCAATT |
| | | TCCTCTTCTTCATACTTTGGAGAAGATAAAGAAGTGGATGAATTTGGTAATCCCATCAAT |
| | | AGTAGTGGAAGTGGAGCAGGGAATTTTGATGGCCGTAATTCTAATAACGGTTTCATCGAT |
| | | TTTAATGCCTCCGCAGATGATGAATTGCAGATGTTGAGAGACGTTGTAGAACAGGGTGCT |
| | | GAAAAACTCGGTAGTTATTTAAGGGACTACTTAAGAAAATAA |
| YGL246C* | RAI1 | ATGGGTGTTAGTGCAAATTTGTTCGTAAAGCAAAGAGGGTCGACAACAGCTCTGAAGCAG |
| | | CCAAAGGAAATAGGATTCTATTCAAGAACTAAGGATGAAGAATATTTAATTAGCGATGAT |
| | | ACGAATTTGAATTACTACTATCTTCCAGACGCTGAATTGGACAGGAAGTTGGATCTTTCA |
| | | TCAGGATTTCAGAAATTTAAGGATTATTACAAAGATTTTGAAGACAGGTGTTCCCTGCGT |
| | | GGGCTATTAGAAACCATAGAAAGCTCTGAAAGACATAAAGGAAAAAAAATTAATGCTGAT |
| | | ATAATTACTTTTAGAGGAATAGCACGGAAATTAATTTCATGTGCATTTGACTCTCCATCT |
| | | TTTAATACAGTTGATCTTCGTATCGTTTCATTCAACGGCCAACTTTTTATCAAGGAAGTG |
| | | CCTGAAGCTGTCAATGCAGCTAAGGCTTCCTCTGCAACTGAAGCAGGGCGTAATATAAAT |
| | | CAAGATCTGAATGTATTTACAGGGTACAAGTTTGAAACTTTGGCTACTCTTTCCAATCCG |
| | | TTGCAGTATACACCCCGTGAAGTGATAGAAAAAAGAACTAAAAGGATTGTATCGCATGGA |
| | | GATGAATACATTAGTGTTGTGAGAACTGGTGTAGGTAACTGCAAGTTAATTCTTGGTGCT |
| | | GAGGTGGATTGTATATTTGACTTTAAAGAAAACGGTAGAGACAATTTAAAACATTATGCC |
| | | GAATTGAAATGTACGCAGCAAGTTGCGAACATATCGGATACGCATAAGTTTGAGCGTAAA |
| | | CTGTTCCGGACCTGGCTGCAATGCTTCTTAGTTGGGATCCCAAGGATCATATATGGTTTT |
| | | AAAGACGACCATTATGTATTAAAAACTGTGGAAGAGTTTTCAACGGAGGAAGTACCTGTT |
| | | TTATTAAAGAACAATAATCCACAAGTTGGAAGTGCATGTTTAGAAGCCATTAAATGGTAT |
| | | GGTCTCCTTACTGAGTGGCTTTTGAAAATGATACCAAGAGATGAGGATCCTCATTCTCAA |
| | | ATAAGAGCATTTAAGTTGGTTTTCGAAAATAATCACCTAAGATTATCCGAAATTGAAGAA |
| | | AGTGATGAGGAATATTCAGGTTTGATTGATGGAGAACATATATTATCGAACGGGTTTAAG |
| | | GAGTGGAGAAAATCTTTGAAATAA |
| YLR139C* | SLS1 | ATGTGGAAATTCAACAAAAAGCTCGCCCGTCTTACTTACCGTCTTTATTCAAGCTCAGGC |
| | | CCAAGCTCGCCCCTTCATGGGAAGAAGAAACTGCCGCAAAATCTGAAATTCGTCGTCTTG |
| | | AACCCTACCCAATCTGGGCTAGTGAAAAATGATCAGAAGCAGCCTAGACACAGGCCGTCT |
| | | AAGAAACGTAGCCATAAGGAAACTGGAGATAACAACCTCGATTTCGGCTCGAAACTCCTT |
| | | GTCTTCGAAAAGCAGAATTCACTAGATTCCGCATTGAACTCCATCCGATTGAAGAAACCG |
| | | ACAAGCGCCAGTCTGCCCTCCCTGGAATACAATGCCCTTCTCCAATCGCTTACATCAAGC |
| | | TACAATCGCTACCAACTGCGCGAGTTCATCTCCACACATCAGCCAGACTCCTCTTCACAT |
| | | CTGACGCACTGGAAGAAAAGCAAGCTGTCCCAATATATAATTGAAAAAATCTGGAATTGC |
| | | CAACCAATTTCAACACCCACCACTCCCACAGGCATCAAGTCCACATCATTGACTTTCCAG |
| | | TTCGATTCTCCTAGAGAAATTTTCCTTTTACTCATCACTCAAAATGGTAAAATACTCACC |
| | | AACTTCAATAAGCTCGGGCTGACATTCATCATCTCCATTCAGGATAACGAATTGACCGTC |
| | | AAGGGTTCGCCTAGCTTGCTCAAATACGCAGAAATATCCTTAAACAAAATATGGTCAAAC |
| | | ATCACTCATGAAAATGTGCGTATGTATTCCCTCATGCCATCAAAAGACGTCATCAACCTT |
| | | ATTCAGAAGGAAACTCACACTTTTTTTGAATACCTCCCGGACTTGCAAATGTACAAGATT |
| | | TCAGCCCTAAGCACCAAGAAAATATCCATGGCCAAAGTCTTTTTATTAAATGCCGTCGCT |
| | | TCTAATCCTAACACAACTCAACACCACCACACAATAGCCTCACCAGCACTCAAGACAGAA |
| | | CTCTATCCGTTCAACAATACTCTAGAGAACTTGGACTGGCTAAACAAGTCACAAGATTGG |
| | | GCAAGATTACAGTCAGTTGTCCCTAAAAATTGCACAGATCTGATGACTCCCACTGAAAAC |
| | | GCCACACCAGAATTGACAGATGCGCAGGTCAGCCAATTCGAATCTTCCTTGTCTAAAAAT |
| | | ATACCATCTCTCTCCCCTTCCGACTCTATTTCCCAATCTTTATCAATAACGCTAGGTCAC |
| | | TCCTTACAGTCCGCGTCTTTTTCTAGTATTTTCCAACCTCTAATACATAAGAGTTTTATT |
| | | TCAAAATTACTGAATCTACCAATGTACAAAGAATCATCATCATCAGCCGTACCTGTGCCT |
| | | GTGCCTCTTGATCAACATCTAATAACAAATGCGCATCAATCCTTCATACAGCTGAACTTC |
| | | ACTCCTGTGCCGCCCACCTCTGGCTCCTCCTCGTCCCCTTTTTTGCAGATTTGGTTCGAG |
| | | ATTGACGAATTCGATAACATTGTTACCACCTCAATGAGGCCTCTTTTGAAATTACAAGAA |
| | | AATTCTGTTATCTTAAGAACTCCACAATGCCAGACAGACTATAAGATCACGTCAGATTAC |
| | | ATTCAAGATCTTTTACCTGATTTCGACCAGACCAATCCGGATGCTTGGTTATCTGAACAA |
| | | AAAGGCCTCCAAGAATTTCTTCTCAAGTCACACTGGAAGTTAAACAAATATCAGAATCTT |
| | | ATGAAAAAAATCAACATTTCTTTACCAGATAACCTAATACAGCAATACCAATTGACTGAT |
| | | GTTTTGACCCATCGTGTCTTGAATTTACGATTTCCTACGAACACTGCCCAAGATGATAAA |
| | | TACATCATTCAATATTCAGATATCAGCCGCGGATTTTTGAACAATGGCTCCTACAGACAA |
| | | TTAGATTTCATCAATGTGAACCCCAGTGAAACCTCACTAAAAACCTTTATCAATGATGTT |
| | | TTAAGCTTTTAA |
| YGR215W | RSM27 | ATGAACGTACCAAAGGCGAGACTCCTAAAAGTAGCAGAGTTATCTGCGAAGATATTTGAT |
| | | CAAAATTTCAACCCATCAGGTATAAGAACCGGTTCTAAGATTCTAAATGAGCGATTAAAG |
| | | GGCCCTTCTGTGGCAAGCTACTACGGCAATCCGGATATCCTGAAGTTCAGACATTTGAAA |
| | | ACATTATATCCTGATATTGAATTTGTAGACCTTGAGGAGCAATATCGATTATCTATGGTA |
| | | GAAGCAAAGAAACGTCGTGGTAAAGGTGCTCCTAAGAAGATGAAGAAGGATGCAGCTGCA |
| | | ACTGCAAAGGGGAAGGGGAAGAAAAAAAAATGA |
| YJL132W | — | ATGAGTATAATTAGTTCTTGGTTACTTGTCTCTATTATTTGCTTAACTACTTCTATTGTA |
| | | ACAAAATTGCAAGCTGCTGGAGTTACTACCCATCTATTTTATTTAACAAGAGGTGCTCCC |
| | | TTAAGTTTAAAGGAAAACTATTATCCTTGGTTGAAAGCTGGTTCTTTCTTTCCGGATGCT |
| | | TTGTACTCATGTGCACCTTCAAATAAGGATTGGTCTGATTTTGCAGAATTCACTCATTGG |
| | | CCCAATTTCTTAATGATTGCAGTATCTTATTGGCAGCAAAAGTATGGTCAGAACGATCGA |
| | | CTTCGGGGAACTCACGGCTCACTCGCACTAAAGTCATTTTTAATAGGGGTATTCACTCAT |
| | | CAGATAGTTGATGTTTCCTGGCATTCGTTAGTTACCGATTATAGAATGCACGGACTACTC |
| | | CGAGTTCTCAGCGAAACAGAATTTGATGGAGATATTGAAACTGCTCATACGTTTTTAGAC |
| | | GTTATGGGTGAATTCCTCACCTTAAATAATGTAATCCGTGACAGTAATAATAACGAAAAT |
| | | TGGGATTTTTTAACTCGCTCTGATTGGAAATTGCCCAGAGAAGAGGATTTAATGGAAATT |
| | | ATAAGGAACGCTGGACTTTCAAAGGAAAAGTTGTCCTACGCAGAACTGGAATTTTGCGTT |
| | | AAAAGAGGAATGGCAGCGGCTATTAGTGAAGGGTACCTATTCCGCTCTCAAAGAAACCAA |
| | | TTGTTAACTAATATTTACTCAACATCCCCCAGGGCTAATGATCTAATACTGAATCATTGG |
| | | CTAGGAGGGCAGTCCAATTTGGTTGCAATGCTCCAAAGATGCGTGCCTTTCTTCGAGACT |
| | | TTGTTTCATGACGAGAATACTAATGAAGCTCAAGCAGAAGAACTGAGATTATGTGCTAAT |
| | | TTACCGCCAGTATCACAAAAACGGATAAATGCACGACCTTTGGTCTCTTCATTAAAAGCA |
| | | CGTAAGGGAAATAGCCACATTGTCGTATCTCCCATGAAATCATTTTCCGATTTTGGCACA |
| | | AGCCTGACTATGGGAAAATTTCGAGAAGACAACAAGGATTATCTCGCTGTAAGTGCACCA |
| | | CTTGAAGACACAGTTGGTGCAATCTATATAGTTCCATGGGATATTCTAACTGTAGCAAGA |
| | | AAAGAGGATTTTAGCATTTTACAACCCATCACTGCCATGTACGGTTCAAAGGTCGGGACA |
| | | TACAAGGCAAGTGATGTTGACTACCTACTCGTTTCACAGCCTGGTACCTGTACAATCGAC |
| | | TTTTACTTCAAAGGAGTAAAAATACTGACAATCAAGGATGAGACCACCGAAGAAGCACAT |
| | | CAGTTACAATTTGCTGTAACAGGTAATTTTTATGATGATAAGATTCCTGATTTGGTGGTG |
| | | TCAAGCCCAAGTTATGGTGCCAACGAAACTGGAATTGCAACATTTATCCCAGGATCTTCC |
| | | ATAATAAGCTATTTGACCAATTCAGACAAATATCAAGTAGTTGATATCTCAACTTTTAAG |
| | | GGCGTTATTAACCTTGACGGGTACCCTATGAAAATACCATTTCAGCATTTCGGGGCAACA |
| | | ATTCAGATATCTGACACGACTGACAAACAAAAGTTAATATATATTACATGTCAAAGTCTT |
| | | GGTACTGTTTTTGTTTATTCGAGTAATGATTTACACGATTTGTCCATCCCAATATATTAT |
| | | ATAACGAAAAATGGCGTCATTCCAGCAAAGGACAGTGACCACGTTGAGTGGCATATAATT |
| | | CCCTCAAAAGAACATGGTATGTTCGGTGCAGCCATATATTCTTGGAATTTTGAAGGTATG |
| | | AGTTTTGTAGCCGTATCTCAACCAATGTTTGACACTGTGTTTATATATATTGAGAAGTCT |
| | | GGGCAAATAGAGTTTTTCTTAAAACTGGTTCTAAAGATTAAGACAAAATCAGATTCAATC |
| | | CCGGATGAGTTTGGTTCATCTTTGCTGTTTAATGACGAAGAAAAGAAATTGTACGTTTCC |
| | | TCCCCAGGCAGTTTCGATGCAAGAGGAAGTATATGGAAAATTAGTATGGATGAGCTATTA |
| | | AAGGCGGGAAACGATCCGAAGCGCAAAACCCTTTTGATTAATAATTTAAGGCATCTCATG |
| | | CTCATAAATCCTGACAAAAGTAGCAAGGGTGTGTCAAATTTTGGTAATAGCATGATTTTG |
| | | GGTCCCCAAAATCACCTTATAGTAGGCATTCCCCAATATGGGTATGGAAATTTTGATCAC |
| | | ATGCAGCTAACCGGTAGAATCCTGGTACTTTAA |
| YHR147C* | MRPL6 | ATGTCATTTATTCAGAGGAGGTTATTGTCGCAAACACTTTTTCTGCGCTCTCAAGTGGGG |
| | | TCTTTACCACTTTATATATCTCCTGAAGTTCAGGTTTCCATAAATGCATTATCCATGCCC |
| | | AGGATTATCAGAAAAGGTAGAACTTCAATGAATATATCACAGAATATCACAGTAAAGGGA |
| | | CCCAAGGGTGAATTGTCCGTGGAGGTCCCTGATTTTTTGCACTTGGACAAGGATGAAAAG |
| | | CACGGTAAAATAAATGTTACAGTTCAGAATTCAGAAGATAAACATCAACGTTCGATGTGG |
| | | GGTACAGTAAGATCACTTATAAATAATCACATTATAGGTGTTACTGAGGGGCATCTTGCT |
| | | GTCCTTAGATTTGTAGGGACAGGTTATAGAGCTCAATTAGAAAATGATGGTAAATTTGTC |
| | | AACGTTAAGGTGGGTGCATCTATCAAACAAGGATTGGACGTTCCTGAAGGTATTGTTGTC |
| | | AAAACACCTGCTCCCACGTCTCTGATTATTGAAGGGTGCAATAAGCAGCAGGTCCTTCTT |
| | | TTTGCTGCCAAATTGAGAAAATTCCATCCACCAGAGCCGTATAAAGGCAAAGGAATATAC |
| | | GTTAACGATGAAACAATAAAGCTAAAGGACAAGAAAATCAAATAA |
| YAL010C* | MDM10 | ATGCTACCCTATATGGACCAAGTACTAAGGGCATTTTATCAGAGCACCCATTGGAGTACG |
| | | CAAAATAGCTACGAGGATATAACGGCCACATCGAGAACATTATTAGATTTCCGAATTCCC |
| | | TCAGCAATACACCTGCAAATTTCCAACAAATCTACTCCCAATACATTCAATTCTTTAGAT |
| | | TTTTCTACGAGGTCCAGGATAAATGGTTCTCTGAGTTATTTATACTCCGATGCACAGCAA |
| | | TTGGAGAAATTCATGCGCAACTCTACTGATATCCCATTACAAGATGCCACCGAAACATAC |
| | | AGACAATTGCAACCAAACCTCAATTTCAGTGTTAGTAGTGCGAATACGTTGAGTAGTGAC |
| | | AACACCACAGTCGACAATGACAAGAAATTACTACATGACTCGAAATTTGTTAAAAAATCC |
| | | CTTTATTATGGTAGAATGTACTACCCCAGCTCTGATTTAGAAGCAATGATAATAAAACGA |
| | | CTAAGTCCACAAACCCAATTTATGCTTAAGGGTGTCAGTAGTTTCAAAGAAAGCTTAAAC |
| | | GTTTTAACGTGCTATTTTCAAAGAGATTCTCACCGCAATTTACAGGAGTGGATATTTTCC |
| | | ACCAGTGATCTATTATGTGGTTATAGAGTATTACACAATTTCCTTACCACGCCTTCCAAG |
| | | TTTAACACCTCACTGTACAATAATTCTTCGTTGTCGCTTGGTGCTGAATTTTGGTTAGGG |
| | | TTAGTAAGTTTAAGCCCCGGTTGTTCGACAACTTTAAGATATTACACACATTCTACAAAC |
| | | ACAGGACGACCACTAACTTTGACATTATCTTGGCAACCATTATTCGGCCATATATCCTCC |
| | | ACATATTCGGCCAAGACAGGGACAAATTCTACTTTTTGCGCGAAGTATGATTTTAATCTT |
| | | TATTCGATTGAATCAAATCTTTCATTTGGGTGCGAATTTTGGCAAAAAAAGCATCATTTG |
| | | CTTGAAACCAATAAAAACAATAATGATAAATTAGAACCAATCTCCGACGAATTGGTTGAT |
| | | ATAAATCCAAACAGCAGAGCGACTAAACTACTGCACGAAAATGTACCGGATCTGAATTCA |
| | | GCTGTTAACGATATTCCTTCTACACTAGATATACCTGTTCACAAACAAAAGCTATTAAAT |
| | | GATTTAACTTATGCATTCTCGTCGTCATTAAGAAAAATCGATGAAGAAAGATCTACCATC |
| | | GAAAAATTTGATAACAAAATAAATAGTTCCATTTTTACCAGTGTTTGGAAATTAAGCACG |
| | | TCATTACGTGACAAGACTTTAAAACTATTATGGGAAGGCAAATGGAGGGGATTTTTAATA |
| | | TCTGCCGGGACAGAGCTGGTATTCACTAGAGGCTTTCAAGAAAGTTTATCCGATGATGAA |
| | | AAGAATGATAATGCAATATCTATATCAGCAACTGATACAGAAAACGGCAATATACCAGTT |
| | | TTCCCGGCAAAGTTTGGCATACAATTCCAGTACTCCACATGA |
| YPL057C* | SUR1 | ATGAGAAAAGAATTAAAATACCTTATCTGCTTCAACATACTCCTCCTGTTATCTATAATA |
| | | TACTACACTTTCGATTTGCTAACGTTGTGTATTGACGATACTGTTAAAGATGCTATACTT |
| | | GAGGAAGACTTAAATCCAGATGCACCTCCAAAGCCTCAACTAATACCTAAAATCATACAT |
| | | CAGACTTATAAAACGGAAGACATCCCTGAGCACTGGAAAGAGGGTAGACAAAAATGTCTC |
| | | GATCTACATCCAGATTACAAGTACATCCTATGGACGGACGAGATGGCCTATGAGTTTATA |
| | | AAGGAAGAATACCCGTGGTTTCTCGATACTTTTGAGAACTACAAATACCCCATAGAACGT |
| | | GCCGATGCCATTCGTTACTTTATCCTGTCCCATTATGGTGGTGTATACATCGATTTAGAT |
| | | GACGGCTGCGAAAGGAAACTAGATCCTTTGTTAGCTTTCCCGGCCTTCTTAAGAAAGACT |
| | | TCACCTTTAGGTGTCTCAAACGATGTCATGGGTTCTGTGCCTAGACATCCCTTCTTTTTA |
| | | AAGGCTTTAAAGTCTTTGAAACACTATGACAAGTACTGGTTTATCCCTTACATGACTATT |
| | | ATGGGGTCTACTGGTCCGTTGTTTTTAAGTGTTATTTGGAAGCAGTACAAAAGATGGCGC |
| | | ATACCTAAGAACGGGACAGTAAGAATCCTACAACCTGCTTACTACAAGATGCATAGTTAT |
| | | TCATTTTTCTCCATTACTAAAGGCTCTTCATGGCATTTAGATGATGCAAAATTGATGAAA |
| | | GCACTAGAAAACCACATCTTATCCTGCGTCGTTACCGGATTTATTTTTGGCTTCTTTATC |
| | | CTATATGGTGAATTCACGTTCTATTGCTGGTTGTGTTCCAAGAATTTCAGTAATCTAACC |
| | | AAAAATTGGAAACTTAATGCTATAAAAGTAAGATTTGTCACAATTCTAAATTCATTAGGC |
| | | TTAAGACTAAAGTTGAGTAAAAGTACCAGTGATACTGCCAGTGCCACTTTGCTGGCAAGG |
| | | CAGCAGAAACGATTGAGGAAAGATTCCAATACAAACATAGTGTTACTAAAATCTTCAAGG |
| | | AAAAGTGATGTTTACGATTTAGAAAAGAATGATTCTTCTAAGTACTCACTGGGAAATAAC |
| | | AGCTCGTAA |
| YOL023W | IFM1 | ATGCTCAGAAGACACGGATTATTCTGGTTAAAGACATGTCCCCGTCTAAATGTTCTTCTA |
| | | AATCAATCCATACCGATACCTCATTTGCTTCATAGCCGTGATATTTGCCAGCAAAGATGG |
| | | TATGCAAAAGGGAAAAGAAGGAACCAGATTTCCAAGAAAGAGTTAAAGCCTCTCAATTTT |
| | | TCTATTCCGAATTATATTTCTGTGAATAAATTGGCCAATTTGTTAAATTGTCGTGTGGAA |
| | | AGATTAATCAAAGATTTGACAGCATTAGGTTTCGAAAATATTACAACCACGTATATATTA |
| | | TCAAAAGAATACGTCGAATTGATTTTACAAGAATACAACTTTGCCCTTCCGAATCTGTCA |
| | | ACGTCTACAAATTTAGATAATGTTTATGATGAGCTAAAATCTCCTGTAAACCCAAAATTA |
| | | TTAACCAAAAGAGCCCCCGTGGTAACTATAATGGGCCATGTTGATCATGGTAAGACCACT |
| | | ATCATAGACTATCTAAGGAAATCTTCTGTTGTGGCTCAAGAACATGGTGGAATCACGCAA |
| | | CACATTGGTGCCTTTCAAATAACAGCTCCGAAGTCTGGGAAAAAGATCACATTTTTAGAC |
| | | ACCCCTGGCCATGCTGCCTTTTTGAAAATGAGAGAAAGAGGTGCAAATATTACTGATATT |
| | | ATTGTTTTGGTGGTATCTGTAGAAGATTCTCTGATGCCCCAGACATTAGAAGCCATAAAA |
| | | CATGCCAAAAATTCAGGAAATGAAATGATAATTGCAATAACGAAAATAGATAGAATTCCA |
| | | CAGCCAAAAGAACGTGAAAAAAAAATTGAAAAAGTTATAAATGATTTGATTGTTCAAGGA |
| | | ATACCAGTAGAGAAAATTGGCGGAGACGTTCAAGTGATCCCGATAAGTGCCAAAACTGGT |
| | | GAGAACATGGACCTTTTAGAGGAATCAATAGTCCTATTAAGTGAAGTAATGGATATAAGA |
| | | GCTGAAAACTCCCCTAAAACTATCGCAGAGGGTTGGATTATCGAAAGTCAAGTTAAAAAA |
| | | CAGGTCGGAAATGTGGCGACCGTCTTGGTTAAGAAGGGCACTTTACAAAAAGGGAAAATT |
| | | CTAATTTGTGGTAATACATTCTGCAAGATCAAAAATTTAATTGATGATAAGGGCATACCG |
| | | ATTCTTAAGGCAACACCATCATATGCTACAGAGGTTCTTGGTTGGAAAGATGTACCCCAT |
| | | GTTGGAGATGAGGTCATTCAGGTAAAGAGCGAAGCGATCGCTAAAAAGTTCATCAGTAAA |
| | | AGGCAAGATCTTATCGAAGTTCAAAAAAACTCTAGTATCGTTGAAAAATTAAATGAAGAA |
| | | AGGGCCCTTGCCAAAGAGCAACACCTAAATAAGGAACTGGAACATGAGAATACTGTCCAG |
| | | GAACACGAACAAAATACTGGTCCTAAACTTATTAATTACATCATCAAATGTGATGTCTCT |
| | | GGCTCAGCAGAAGCGGTTTCAGAAAGCATTTCCTCTCTAGGAAATGACGAAGTGAGATGT |
| | | AATGTTATCTCTTCTTCTGTTGGTATACCTACGGAAAGTGACTTAAAGATGGCGCAAATA |
| | | ACGGAGAGTACAATTCTATGCTTCAATTTGGGGAATTTGCCGAGTGAGGTGATCAACAAT |
| | | CGTGCTGGCATCAAAATAAAACAGTACAATGTTATCTATAAGCTAATTGAAGACGTCACT |
| | | GAAACACTAACTGAAAATCTAAAGCCCATCTTTGAAAAAAAGATCGTTTCAACTGTTGAC |
| | | GTTCGTGAAACTTTTGACTTTAGGTTAAAGAAAAAAATTATTAGGATTGCAGGTTGCAAA |
| | | GTAAACAACGGTGTCATCAAAAAGAATTCATTGGTGCAGGTAGTTAGAGGTCCTAACGAA |
| | | GATGTAATTTTTGATGGGAAAATCTCTACTTTGAAGCACAATAAAGATGACGTAGCAGAA |
| | | GTTTCAAAAGGGCATGAATGTGGAATCACTTTTGAGAGTGGATTCGAGGGTTTCAAACCA |
| | | GGTGATAAGATCCTAGTTTATGAAAACGTGCGAGTTCCTCGTTACTTGTAA |
| YBR162W-A | YSY6 | ATGGCCGTACAGACACCAAGACAAAGACTGGCTAATGCCAAGTTTAACAAGAATAACGAA |
| | | AAGTATAGAAAATACGGTAAGAAGAAGGAGGGCAAAACTGAGAAAACCGCACCTGTGATA |
| | | TCCAAAACTTGGTTGGGTATTCTTCTGTTTCTTCTCGTAGGTGGTGGTGTTTTGCAACTA |
| | | ATCAGCTATATCCTATGA |
| YOR205C* | LRC5 | ATGTTGAATCTGTGTCATGCTCTTCGAGGCGTACGTCAGTTTTCCTGTTCTGTGATTGTG |
| | | AAAGTGAAATGTGCGTCATGTTCCATAAAACTACAGGATCAGGATCCTAGTAAACCAGGA |
| | | TATTATACGAAGCCAAAAAGTCTACCCGACTCAAAACTAAATCCTGACCTCCAAGATCTG |
| | | AAGTATTTGCTATTCAGTCAAGACATACAGCTCTCGAAACAGGCAACTCAAAATGATCCT |
| | | GACCTCAAAACCAAAAGAGATTTACTATTGAGAGTGATTTGCAAAAGGTGCAGTAATGCC |
| | | CTTCATCATAACAATTATAACCCGGAAGAGTTTCCCGAAAGCACATTAAATGATATTCTG |
| | | AACTACGTCCCCAGAGGCTCAAATGTTATGCATATTGTCCCCTTTGTAGAATTTCCCTTA |
| | | CATTTAGACCCCAATGTTTTGAAACGAAATGATTTAGATACTACATTAGTGTTAACGAAA |
| | | AGCGATCAAGTTTTTAAAGATAAAAATGCTGTCAGCAAAAAGGTTCCTATATTCATGAAG |
| | | CAATTTTTAAAGAACACTTTAAGAATTGACTCAAATAAAACATTCGCCATATCTGCGCTG |
| | | AAAAACTGGAACATATCCATGTTTTACAACTACTTTAAAAACTACACATATTTGCTAGGC |
| | | AACCCTAATGTTGGTAAATCAACTTTGATCAATACTCTACTACAGAAATATCTTGGATAT |
| | | AAGGTTAAAATTGATTCTACTGGGAAAATTAACTCCCCCTCTGAAGAAGTGATGCAAGAA |
| | | GCTTTTACGAACCCGAAGAATTTTTTCAAGATTCAAGCTGCTGGTGTATCACATATTCCC |
| | | AATTTAACTAGATCCGTACAAGCCTACCAAGTTGGAGGTAAAATACTTTTTGATCTTCCA |
| | | GGCTATTCAACTTCCACCTCCAGATTACGCCTAGAAGAACTAATCGACGAGCGTTGGCTT |
| | | CAAAGGCTGCGAAAAACCGATTTATTCAATCGGAAGCATATAAAACAGAAGACTTATGAG |
| | | TCTATGAAGGGCACATCACAGGGAGGCTGTTATACCGTGGGAGGGATTTTCTACCTGGTA |
| | | CCTCCGAAAGGATCCATAAATCAGATTGTGAAATACATACCAGGACCATCAAAAACATTC |
| | | AAGAACATTGAAAAAGGTATTGATGTATTCAACTCGTGCAATTCATCATCTGGCACACAT |
| | | CCGTTATCACGGTATTGTGGAATTAAAAGCGTTATATGCGAAAAAAGCCAGTACAAAAGG |
| | | TACGCAATACCTCCATTTATTGGAAGTATAGAGATCGTTCTGAAAGATATAGGATATATT |
| | | TTATTAAGGACAACTGGGAGGTATGAGTTCAAAGGACTACATGAGATATGGATACCTAGG |
| | | GGTATCCAAGTAGGTATAAGGGAACCGCTAGAAAACTTGATTGAGTCTGGCTACCAGCGT |
| | | TACATTGAAACAAATGGTAAAGAGTCAAGCTGTCCCAGAGATAGACCGATCATCAGTTCC |
| | | CTGTATGAGATGGCCCCGGATGAAGCTGATACTTTGAACGCAGTGAAGAAGTCGTATTTA |
| | | GAAAAAACCGAGAAAGATTTATCTGCTAGAAGATTTGTCGACGATGATCCTTATGATTTG |
| | | GTTCAACACCTAGAAAAAAAAAAGAATCCCTATTGGTACTACCAATGGTAA |
| YDL067C* | COX9 | ATGACTATTGCTCCAATTACTGGTACGATCAAGAGAAGAGTCATCATGGACATCGTCCTC |
| | | GGGTTCTCCCTCGGGGGTGTCATGGCCTCTTACTGGTGGTGGGGATTCCACATGGATAAG |
| | | ATTAACAAGAGAGAGAAGTTCTACGCAGAGCTAGCTGAGAGGAAAAAGCAAGAGAACTGA |
| YDL050C* | — | ATGATAATGAGATACGAGAACCAGAAGAAACACAAGAATCACTCCTTGTGCTCCTCATCG |
| | | TCATCGGCAATGGCAGAAGAGTCCTCTTTTGACTCTTCCTTGCCGTTTTTCTTCTTATTT |
| | | TTGGGGAATTTAGGCAAGTTTTTCTTCTTATGGCCGTTAAAGGATCTTGATCTACCGCTG |
| | | AAGTTTTTGGACTTCGAGGCCTCTCTCTGTAAATCAAACTGTTTTTTCGTCAAAACACTC |
| | | AGTTTCTTACCTTCATATGACAAAATTTCGTTGGACTCATCGTCATTGGAATACGATTTC |
| | | AAAAAAGCTTCACATTCTGGAATTGTCTTAAATTCCACCAAGACCGTACCATTAAATTTC |
| | | TTGTTTCTGTGA |
| YJL189W | RPL39 | ATGGCTGCTCAAAAGTCTTTCAGAATCAAGCAAAAAATGGCTAAGGCTAAGAAGCAAAAC |
| | | AGACCATTGCCACAATGGATCAGATTGAGAACCAACAACACTATCCGTTACAACGCTAAG |
| | | AGAAGAAACTGGAGAAGAACCAAGATGAACATCTAA |
| YCR028C-A* | RIM1 | ATGTTTTTACGTACTCAAGCTCGTTTCTTCCATGCTACTACCAAGAAGATGGACTTCTCG |
| | | AAAATGTCCATCGTCGGCCGCATTGGCTCTGAATTCACTGAACATACTTCTGCTAATAAC |
| | | AATCGTTATTTGAAATATAGTATCGCTTCGCAACCAAGAAGAGATGGCCAAACCAATTGG |
| | | TATAATATCACCGTTTTCAATGAACCTCAAATCAATTTTTTGACAGAATATGTTAGAAAA |
| | | GGCGCTTTGGTATATGTTGAAGCAGATGCTGCTAACTATGTCTTCGAGAGAGACGACGGT |
| | | TCTAAGGGTACTACTTTGAGCTTAGTTCAAAAGGACATTAATTTATTGAAGAATGGGAAG |
| | | AAATTAGAAGATGCTGAGGGCCAAGAAAATGCTGCTTCTTCAGAATAA |
| YGR220C* | MRPL9 | ATGTCGAAGTTCCTACAGGGATCAATATTTAGCATATCAAAACTTCATGTTAGATATTCT |
| | | TCTACTCGGCCTTTTCTAGTGGCTCCTAGTATTGCTAACAGTATAACAACCGAGGCTCCA |
| | | GCGATTAATCACTCACCTGAACTGGCAAATGCGAGAAAATGGCTACCAAAAAGATGCGGT |
| | | TTAATTACTCGTAAGAAGGGAATGATGCCTTATTTTGATAAATCTACTGGTGAAAGAAGC |
| | | GCCGCTACTATATTAGAAGTTAATAATGTTGAAGTAATAATGCATAGAACTTCGGAGGTC |
| | | AATGGGTACTTCGCGTGTCAAGTAGGGTACGGATCAAGGCATTTGTCTAAAGTTAGCAGA |
| | | CAAATGCTTGGTCATTTCGCTTCTAAAGTTGTTAACCCAAAAGAGCATGTGGCCGAATTC |
| | | CGAGTTAAAGATGAAAAGGGATTAATACCACCTGGCACATTATTGAAACCCTCTTTTTTC |
| | | AAAGAAGGCCAATATGTTGACGTCAGATCAGTCAGCAAAGGCAAAGGTTTTACTGGTGTG |
| | | ATGAAAAGGTATGGATTCAAGGGTTTGAGAGCATCTCATGGTACATCTATTATGCATAGA |
| | | CACGGTGGTTCTTATGGTCAAAACCAAGACCCAGGTAGGGTACTGCCAGGAAGAAAGATG |
| | | CCTGGTCATATGGGAAACGAGCATGTTACTATCCAAAATGTTAAAGTTTTGAAAGTCGAC |
| | | GATGAAAATAATGTTATTTGGGTTAAAGGCTCTGTGGCTGGGCCCAAAAACAGTTTCGTT |
| | | AAAATTCAAGATGCAATCAAAAAAACTTGA |
| YBR101C* | FES1 | ATGGAAAAGCTATTACAGTGGTCTATTGCGAATTCTCAAGGGGACAAAGAAGCTATGGCT |
| | | AGGGCCGGCCAACCTGATCCTAAATTGCTACAGCAGTTATTCGGTGGTGGTGGTCCTGAC |
| | | GATCCAACCTTAATGAAAGAATCCATGGCTGTTATTATGAATCCGGAGGTTGACTTAGAA |
| | | ACAAAACTCGTTGCATTTGACAACTTTGAAATGTTGATTGAGAACTTAGATAATGCTAAT |
| | | AATATCGAAAATTTAAAACTGTGGGAGCCATTGTTGGATGTTCTTGTTCAGACGAAGGAT |
| | | GAAGAACTACGTGCTGCTGCTTTATCCATTATTGGAACGGCTGTGCAAAACAACTTGGAT |
| | | TCGCAAAATAATTTCATGAAATACGACAATGGTCTGCGAAGCCTTATCGAAATAGCTAGT |
| | | GACAAGACAAAGCCACTCGACGTGAGAACAAAAGCTTTTTACGCACTATCTAATCTAATA |
| | | AGAAACCACAAAGATATCTCAGAAAAGTTTTTCAAATTAAATGGGCTCGACTGCATAGCA |
| | | CCTGTATTAAGTGATAACACCGCCAAACCAAAACTGAAAATGAGAGCCATTGCCTTATTG |
| | | ACCGCATATTTGTCATCTGTTAAGATTGATGAAAATATAATCAGTGTGCTGAGAAAGGAT |
| | | GGAGTAATTGAAAGTACGATTGAGTGCTTGTCTGACGAGAGTAACTTGAACATCATAGAT |
| | | AGAGTTCTGTCTTTTCTCTCTCACCTGATATCTTCCGGAATAAAATTTAATGAACAGGAA |
| | | TTGCACAAATTGAACGAAGGTTACAAACATATCGAGCCTCTAAAGGACAGACTTAATGAA |
| | | GACGATTATTTAGCCGTAAAGTATGTATTATGA |
| YJL208C* | NUC1 | ATGTGCAGTAGGATACTCTTGTCCGGTTTAGTCGGACTGGGTGCTGGTACTGGCTTAACC |
| | | TATCTTCTTTTGAACAAACATTCTCCAACGCAAATCATTGAGACACCTTATCCACCTACC |
| | | CAGAAACCTAATAGTAATATTCAATCTCACTCTTTCAACGTCGATCCTTCCGGGTTCTTC |
| | | AAGTATGGTTTTCCTGGTCCGATTCATGATCTGCAGAACCGCGAAGAGTTTATCTCATGT |
| | | TACAACAGACAAACTCAAAATCCTTATTGGGTCCTCGAACATATAACGCCAGAATCATTG |
| | | GCTGCAAGGAATGCTGACAGAAAAAATTCCTTTTTCAAGGAAGATGAAGTAATTCCAGAA |
| | | AAGTTTAGAGGTAAACTAAGAGACTATTTTAGGTCGGGCTATGATCGAGGCCATCAAGCC |
| | | CCAGCTGCAGACGCAAAATTTTCTCAACAGGCCATGGATGATACATTCTACTTATCCAAT |
| | | ATGTGTCCTCAAGTAGGAGAAGGTTTTAATAGAGACTATTGGGCGCATTTGGAGTACTTT |
| | | TGTAGGGGATTGACTAAGAAATATAAGAGTGTAAGAATCGTGACTGGTCCATTGTATCTA |
| | | CCCAAAAAGGATCCCATAGATAATAAATTTAGGGTTAATTATGAAGTTATTGGCAATCCA |
| | | CCCAGTATTGCTGTTCCAACGCACTTTTTTAAATTGATTGTTGCAGAAGCACCAACAGCC |
| | | AATCCTGCTAGAGAGGATATTGCTGTCGCGGCATTTGTATTGCCAAACGAACCGATATCA |
| | | AATGAGACGAAATTGACTGACTTTGAAGTTCCTATAGATGCTTTAGAGAGAAGTACTGGG |
| | | CTAGAACTTCTGCAAAAAGTACCACCTTCAAAGAAGAAGGCATTATGCAAAGAGGTAAAT |
| | | TGTCAAATTGTAGTGAGAGATTTCTCTAACGCGGCGATCAAACAATCGAAAGATGTGAAA |
| | | TTGTTACCTCCTCCAAAAAAAAGGAATTGA |
| YBR144C* | — | ATGCTTTTTGACTCATCCTTCCGCGAAAGGAGAACTTTTTACAATGCCGCCGGCTCTGTT |
| | | AGCGTACCCTTCTACAAGCAGATAGCAGAACAAACACATGATATATTCAAAAGGTGCAAT |
| | | GTCAGAGAACATATATTGCGCCCCTGTCCTGTAGACATCAGTCATTTTTCGCGGGACTTG |
| | | AATGGCGCACCATTATCCTATACAGAAAACATTAAATTTACTGCAAAATTTTGGGCAAAC |
| | | GCTTGGATAGACTATGAATCCGTCATGAAGACACTACTTGTAAAATTATATATATCCTTT |
| | | TTTTTCATGTGCTAG |
| YGL251C* | HFM1 | ATGAAAACAAAGTTTGATCGCCTCGGTACAGGAAAAAGAAGTAGACCCTCTCCAAATAAT |
| | | ATTGACTTTAACGACCAGTCTGCTACATTTAAAAGAAATAAGAAAAATAGCCGCCAACCA |
| | | AGTTTTAAAGTAGGTTTGTCTTATAACAGTTTACTGGATGATTGTGATGATGAAAATGAA |
| | | ACAGAGGAAATATTTGAGGGCCGTGGTCTGCAATTCTTCGACAAAGACGATAATTTTTCA |
| | | ATAACAGCAGATGATACACAAGTAACTAGTAAATTATTTGATCACGATTTAGAGCAGACA |
| | | CCTGATGAAGAGGCGAAGAAACCTAAGAAGGTCACGATAAGGAAATCAGCGAAGAAATGT |
| | | TTGAGCACGACCATATTACCAGATTCATTCAGAGGGGTTTTTAAATTTACAGAATTTAAT |
| | | AAAATGCAATCAGAGGCTTTTCCTAGTATTTATGAGAGTAACGAGAACTGCATAATTTCT |
| | | TCACCAACCGGATCAGGCAAGACTGTATTATTTGAATTGGCAATATTACGTCTTATAAAG |
| | | GAAACAAATAGTGATACCAATAACACCAAAATTATATACATCGCGCCAACCAAATCTCTA |
| | | TGTTACGAGATGTACAAGAACTGGTTTCCTTCCTTTGTGAATCTTTCTGTTGGTATGCTT |
| | | ACTAGTGATACCTCTTTTCTGGAAACTGAAAAGGCTAAAAAATGTAACATAATAATTACG |
| | | ACACCAGAAAAATGGGATTTGTTAACAAGAAGATGGTCCGATTACAGCCGATTATTCGAA |
| | | TTGGTCAAACTTGTCCTAGTTGATGAGATTCATACCATAAAGGAGAAAAGAGGAGCATCT |
| | | TTGGAAGTAATTCTGACAAGGATGAATACAATGTGTCAAAACATTCGGTTTGTTGCTTTA |
| | | AGTGCAACAGTTCCAAATATAGAAGACCTAGCATTGTGGCTCAAAACTAACAACGAGCTT |
| | | CCTGCAAATATTCTTTCTTTTGACGAATCGTACAGGCAAGTTCAGTTAACGAAGTTTGTT |
| | | TATGGGTACTCCTTCAATTGCAAAAATGACTTCCAAAAAGATGCTATATATAATTCCAAA |
| | | TTGATTGAAATAATTGAAAAGCATGCCGATAATCGTCCCGTACTAATATTTTGTCCGACT |
| | | AGGGCTTCAACTATATCAACAGCAAAATTTTTATTAAATAATCATATTTTTTCAAAAAGT |
| | | AAGAAAAGATGTAATCATAATCCCTCCGATAAAATATTAAATGAATGTATGCAACAAGGT |
| | | ATCGCTTTCCATCATGCTGGAATTTCTTTAGAAGACCGTACTGCCGTTGAGAAAGAATTT |
| | | CTTGCAGGTTCAATTAATATATTGTGTTCAACTTCAACGTTAGCTGTTGGTGTGAACCTT |
| | | CCCGCATATTTGGTCATTATCAAAGGCACCAAAAGTTGGAACTCTTCTGAAATCCAAGAG |
| | | TATTCAGACCTAGATGTACTGCAAATGATTGGGCGGGCAGGAAGACCTCAATTTGAAACT |
| | | CACGGGTGCGCAGTAATCATGACAGACTCTAAAATGAAGCAAACATATGAAAACTTAATT |
| | | CATGGAACTGACGTTTTGGAAAGTTCATTACATTTGAATTTGATCGAGCATTTAGCAGCA |
| | | GAAACCTCTTTGGAAACTGTATATTCAATTGAAACTGCAGTAAATTGGCTACGAAACACA |
| | | TTCTTTTACGTTAGATTTGGGAAGAATCCAGCTGCATATCAAGAGGTGAACAGATACGTT |
| | | AGTTTTCATTCCGTTGAAGACTCTCAGATAAATCAATTCTGTCAGTATTTGCTTGACACC |
| | | TTAGTAAAAGTGAAAATAATCGATATCAGCAATGGGGAGTACAAATCTACAGCGTACGGA |
| | | AATGCAATGACGCGACATTATATATCCTTTGAGTCGATGAAACAATTCATTAACGCAAAA |
| | | AAATTTCTATCATTACAAGGGATACTAAATTTACTGGCCACTTCTGAAGAGTTTTCAGTT |
| | | ATGAGGGTGAGGCACAATGAAAAAAAATTGTTCAAAGAAATCAATTTATCGCCACTTTTG |
| | | AAATATCCATTTTTAACCGAGAAGAAACAGAGCCAAATAATTGACAGGGTTAGTCAAAAG |
| | | GTTTCTCTTTTGATTCAATATGAGTTAGGTGGGTTAGAATTTCCATCATACGAAGGCGCT |
| | | TCTAAACTACATCAAACTCTTGTTCAGGATAAATTCCTCGTTTTTAGACATTGTTTTAGA |
| | | TTACTAAAATGCATGGTAGACACATTTATTGAAAAAAGTGATGGAACATCACTGAAAAAT |
| | | ACTTTGTTCTTACTGCGAAGCCTGAATGGTCACTGCTGGGAAAACACGCCAATGGTTCTT |
| | | AGGCAATTGAAAACAATTGGACTAGTTTCAGTAAGAAGGCTAATACGACATGGAATTACC |
| | | AATCTAGAAGAAATGGGGCATTTGTCAGATACTCAGATTGAATACTATCTTAACCTCAAG |
| | | ATTGGAAACGGCATAAAAATTAAAAACGACATTTCTTTGTTGCCTTGTCTGAATATTAGA |
| | | ACAAAACTAGAAAATTGCAAAATAGAAAATGAAGAGTTGTGGTTGACATTTAAAGTTGAG |
| | | ATTAGTGCAACATTCAAGTCTAGCATTTGGCATGGTCAGCACCTCTCATTAGATATCGAA |
| | | ACCGAGAAAAGCTCAGGAGAGTTAATCGACTTTAGAAGATTGCAAGTTAACAAACTACAG |
| | | TCACCAAGGGGCTTTAGAATTTCAGCAAAAATATCACCAAAACTAGAAAAAATAGAATTC |
| | | TCAATTCATTGCCAAGAAATCGCCGGACTCGGCAAAACAATCGTATATTCCACTGATCAT |
| | | TTGGCATCACAGTTCTCTGCGAAGACACCAAATATAAGGAAAGATCTAAATAGTTTAGAA |
| | | AAATGCCTCTTCTATGAAAGCAGTAGTGACGGAGAAGTGGGGAAAACGTCTAGGGTTTCC |
| | | CATAAAGATGGATTAGAAGAATCGCTATCCTCAGATGATAGTATACTAGATTATTTGAAT |
| | | GAAAGGAAGAAATCATCAAAGGCGGTTGAATCTGCAGCAGTTATTCACCCGGAGGCACAC |
| | | TCTTCTTCACATTTTAGTAATGGCCGCCAAGTAAGATCTAATGGCAATTACGAGTGTTTT |
| | | CATAGCTGCAAAGACAAAACGCAATGCAGACATTTATGTTGTAAAGAGGGTATTCCAGTA |
| | | AAGTATATAAAAGAAAAAGGACCCTCCTCAATCAAGCCAGTATCAAAACCTGATCAAATA |
| | | CGCCAGCCACTGCTTGCAAAAAATATCAACACAACCCCTCACCTGGAAAAAAGACTGAAT |
| | | AGTAAACCAAAGCAGTGGCAAGAAGAAAATACAGATATTGCGACAGTTCATACACTTCCT |
| | | TCAAAAATTTATAATCTATCGCAACAGATGTCGTCTATGGAAGCGGGTGAACAAGTCCTA |
| | | AAATCAGGCCCCGAAAACTGCCCTGAAATAATTCCTATTGATCTTGAATCATCCGATAGT |
| | | TATTCTAGTAATACAGCGGCGAGCTCAATAAGTGATCCGAATGGTGATCTGGATTTCTTG |
| | | GGTTCCGATATAGAGTTTGAATGA |

TABLE 8
Resistance Genes

| Systematic | Common | |
|---|---|---|
| Name | Name | Sequence |
| YAL002W | VPS8 | ATGGAGCAAAATGGCCTTGACCACGACAGCAGATCTAGCATCGATACGACTATTAATGAC |
| | | ACTCAAAAGACTTTCCTAGAATTTAGATCGTATACCCAATTAAGTGAAAAACTGGCATCT |
| | | AGTTCTTCATATACGGCACCTCCCCTGAACGAAGATGGTCCTAAAGGGGTAGCTTCTGCA |
| | | GTGTCACAAGGCTCCGAATCCGTAGTCTCATGGACAACTTTAACACACGTATATTCCATC |
| | | CTGGGTGCTTATGGAGGGCCCACGTGCTTGTATCCGACAGCCACGTATTTTTTGATGGGC |
| | | ACTTCTAAAGGATGCGTACTCATTTTTAATTATAATGAACATTTGCAGACAATCCTAGTG |
| | | CCGACCTTATCTGAGGACCCTTCTATTCACTCAATAAGAAGTCCAGTGAAATCAATTGTC |
| | | ATATGTTCCGATGGTACTCATGTAGCTGCCTCATACGAGACCGGAAATATATGCATTTGG |
| | | AACTTGAACGTAGGGTATAGAGTGAAACCCACTTCTGAACCAACAAATGGTATGACCCCA |
| | | ACGCCTGCCTTACCGGCAGTCTTACACATCGATGACCATGTGAACAAGGAAATCACAGGG |
| | | TTAGACTTTTTTGGTGCTCGGCATACAGCCCTGATTGTTAGTGATAGGACAGGTAAAGTA |
| | | TCACTCTATAACGGTTACAGAAGAGGCTTTTGGCAGTTGGTGTATAATTCAAAAAAAATT |
| | | TTAGATGTGAACTCTTCCAAGGAGAAATTAATAAGGTCAAAGTTGTCTCCACTAATATCA |
| | | CGGGAGAAAATTTCCACTAATTTGTTGAGTGTACTCACAACTACACATTTTGCCCTTATT |
| | | TTATTATCGCCACACGTTTCTTTGATGTTTCAAGAAACTGTTGAACCCTCAGTACAAAAT |
| | | TCTCTAGTCGTGAATAGCTCTATTTCATGGACTCAAAACTGTTCCAGGGTTGCTTATTCC |
| | | GTAAATAATAAAATTTCTGTTATTTCCATATCTTCATCAGACTTCAATGTTCAGTCCGCT |
| | | AGCCATTCTCCTGAATTTGCAGAATCTATATTATCCATTCAATGGATTGACCAGCTCCTA |
| | | CTTGGTGTTTTAACCATATCGCACCAATTTTTGGTATTGCACCCCCAACATGACTTCAAG |
| | | ATCCTGTTAAGATTGGATTTTCTGATTCACGATTTGATGATCCCACCTAATAAATATTTT |
| | | GTAATAAGTAGAAGAAGTTTCTACCTGTTAACAAACTACTCATTTAAAATTGGCAAATTT |
| | | GTGTCTTGGTCAGATATTACTTTAAGACATATTTTGAAAGGCGACTACTTGGGTGCATTG |
| | | GAGTTCATAGAATCACTTTTGCAACCTTACTGTCCACTGGCAAACTTGTTGAAGCTAGAT |
| | | AATAATACGGAAGAGAGGACTAAGCAACTTATGGAACCATTTTACAATCTGTCCTTGGCT |
| | | GCCCTAAGGTTTCTTATAAAAAAAGATAATGCCGACTACAATAGGGTTTACCAATTATTA |
| | | ATGGTAGTTGTTCGTGTTTTGCAGCAATCTTCCAAAAAACTAGACTCAATTCCTTCTCTA |
| | | GACGTCTTTTTGGAACAGGGTCTGGAGTTCTTTGAATTGAAGGACAACGCGGTATATTTT |
| | | GAAGTTGTAGCAAATATTGTTGCCCAAGGATCAGTTACGTCAATTTCCCCAGTTCTTTTC |
| | | AGGTCCATAATTGATTACTATGCTAAGGAGGAGAATTTAAAAGTAATTGAAGACTTAATC |
| | | ATCATGTTAAATCCTACTACGCTTGATGTTGATCTTGCCGTCAAACTATGCCAAAAGTAT |
| | | AATTTGTTCGATTTATTAATATATATTTGGAACAAGATCTTTGATGATTATCAAACCCCA |
| | | GTGGTGGACTTGATATACAGGATTTCTAACCAAAGTGAAAAATGTGTGATCTTCAATGGT |
| | | CCTCAAGTACCTCCTGAAACGACTATATTTGATTACGTAACGTATATCCTTACTGGCAGG |
| | | CAATATCCACAAAACTTGTCTATATCACCAAGTGATAAATGCTCCAAAATACAAAGGGAA |
| | | CTTTCAGCATTTATTTTTAGTGGCTTCTCCATAAAATGGCCGTCGAACAGCAATCATAAA |
| | | CTTTACATATGCGAAAATCCAGAAGAAGAGCCAGCATTTCCTTACTTTCACCTTTTATTG |
| | | AAATCGAATCCGAGTAGGTTCTTAGCAATGCTCAATGAAGTGTTTGAAGCGTCCTTGTTT |
| | | AACGATGACAATGACATGGTTGCATCAGTTGGAGAAGCAGAATTGGTAAGTAGGCAATAT |
| | | GTTATTGATCTACTATTGGATGCTATGAAAGATACGGGAAATTCAGACAACATCAGGGTA |
| | | CTTGTTGCAATTTTCATTGCAACTAGTATATCAAAATATCCTCAATTTATTAAAGTGTCT |
| | | AACCAAGCCCTCGACTGCGTTGTTAATACCATATGCTCCTCTAGGGTTCAAGGTATATAT |
| | | GAAATTTCTCAAATAGCTCTGGAGTCGCTTTTACCCTATTATCATTCAAGAACAACAGAA |
| | | AATTTTATACTGGAACTAAAAGAAAAAAATTTCAATAAAGTTCTTTTCCATATCTATAAA |
| | | AGTGAAAATAAGTACGCCAGTGCGCTTTCACTTATTTTAGAAACTAAGGACATCGAAAAA |
| | | GAATATAACACGGACATTGTATCCATAACCGACTACATACTCAAAAAATGCCCACCTGGA |
| | | AGTTTAGAATGTGGCAAAGTTACTGAAGTTATCGAGACGAACTTTGATCTTCTTCTCTCA |
| | | AGGATCGGTATCGAAAAATGCGTCACAATTTTTTCTGACTTTGACTATAATCTTCATCAA |
| | | GAAATCCTGGAAGTAAAGAATGAGGAGACTCAGCAAAAGTATTTGGATAAGCTTTTTTCT |
| | | ACGCCAAATATCAACAATAAGGTCGATAAGCGTTTAAGAAATTTACACATCGAATTGAAC |
| | | TGTAAATACAAGAGCAAAAGGGAAATGATTCTTTGGCTTAATGGTACAGTTCTCAGCAAC |
| | | GCTGAGAGCTTACAAATTCTGGACTTATTGAATCAAGACTCTAATTTTGAAGCTGCAGCT |
| | | ATAATTCACGAACGCTTGGAAAGTTTTAACCTAGCAGTCAGGGATTTATTAAGTTTTATT |
| | | GAACAATGTCTAAATGAAGGGAAAACAAATATATCTACTTTATTGGAATCTTTGAGGAGG |
| | | GCCTTTGATGATTGTAATTCTGCTGGTACCGAGAAAAAATCGTGTTGGATATTATTGATT |
| | | ACATTCCTGATCACTCTATATGGGAAATATCCTTCACACGATGAAAGGAAAGATTTATGT |
| | | AATAAACTACTTCAAGAAGCATTTTTGGGATTGGTTAGGTCCAAGAGTTCCTCTCAGAAG |
| | | GATTCAGGTGGGGAATTCTGGGAAATAATGTCTTCTGTTCTTGAGCACCAAGACGTTATT |
| | | TTAATGAAAGTTCAGGATTTAAAGCAACTGCTACTGAATGTTTTTAATACTTATAAATTG |
| | | GAAAGATCTCTTTCTGAGTTGATTCAAAAGATTATAGAGGATTCTTCGCAAGATCTTGTT |
| | | CAACAGTATAGAAAATTTCTGAGTGAAGGGTGGTCTATACACACCGACGACTGCGAAATC |
| | | TGCGGGAAAAAAATATGGGGAGCTGGTCTGGACCCATTACTTTTTCTAGCTTGGGAAAAT |
| | | GTACAGCGCCACCAAGATATGATTAGTGTAGATCTCAAAACTCCCCTTGTCATATTCAAA |
| | | TGTCACCATGGCTTTCACCAGACTTGCCTCGAAAACTTGGCCCAGAAACCCGATGAATAT |
| | | TCTTGTTTAATTTGCCAGACGGAATCTAACCCAAAAATAGTATAA |
| YAL013W | DEP1 | ATGAGTCAGCAAACACCACAGGAAAGTGAACAGACCACAGCGAAAGAACAGGACCTTGAT |
| | | CAAGAGAGCGTGTTGAGCAACATTGACTTCAATACGGATTTGAATCACAATTTGAATTTA |
| | | TCGGAATACTGTATATCCAGTGACGCAGGAACAGAGAAGATGGATAGCGACGAGGAGAAG |
| | | TCGTTGGCCAATCTGCCGGAGTTGAAATACGCTCCCAAGCTATCCAGCCTGGTGAAGCAA |
| | | GAGACGCTCACCGAGAGCTTGAAAAGACCACACGAAGATGAGAAAGAGGCGATAGATGAG |
| | | GCCAAGAAGATGAAAGTGCCGGGAGAGAACGAGGACGAAAGCAAGGAAGAGGAAAAGAGT |
| | | CAAGAACTGGAAGAGGCAATTGACAGCAAGGAGAAGAGCACCGACGCCAGGGACGAGCAA |
| | | GGGGACGAAGGTGATAATGAGGAGGAAAACAACGAGGAGGATAATGAAAACGAAAACGAG |
| | | CATACAGCACCGCCTGCGCTGGTGATGCCCTCCCCCATCGAAATGGAGGAACAGAGGATG |
| | | ACTGCGCTGAAGGAAATCACCGACATCGAGTACAAGTTCGCGCAATTGCGCCAAAAACTA |
| | | TATGACAATCAATTGGTGCGGTTGCAAACGGAGCTGCAGATGTGTCTGGAAGGGTCACAC |
| | | CCGGAATTGCAGGTCTACTACTCGAAGATTGCCGCGATCCGTGACTACAAGCTACACCGA |
| | | GCGTACCAGCGACAGAAGTACGAGCTTTCATGCATCAACACAGAAACAATCGCTACCAGG |
| | | ACATTCATTCACCAGGACTTCCACAAGAAGGTCACCGACCTGCGAGCCAGGCTGCTGAAC |
| | | AGAACCACGCAGACCTGGTACGATATCAACAAGGAGCGCCGCGATATGGATATAGTCATC |
| | | CCAGATGTCAATTACCACGTCCCCATCAAACTTGATAACAAGACGCTGAGCTGTATCACG |
| | | GGCTACGCCAGCGCAGCACAGCTGTGCTATCCCGGCGAGCCCGTGGCAGAGGACCTCGCT |
| | | TGCGAAAGCATCGAGTACCGCTACAGAGCCAACCCGGTGGACAAACTCGAAGTCATTGTG |
| | | GACCGAATGAGGCTCAATAACGAGATTAGCGACCTCGAAGGCCTGCGCAAATATTTCCAC |
| | | TCCTTCCCGGGTGCTCCTGAGTTGAACCCGCTTAGAGACTCCGAAATCAACGACGACTTC |
| | | CACCAGTGGGCCCAGTGTGACCGCCACACTGGACCCCATACCACTTCTTTTTGTTATTCT |
| | | TAA |
| YAL021C* | CCR4 | ATGAACGACCCTTCTTTACTAGGCTACCCTAATGTGGGCCCGCAGCAGCAACAGCAACAG |
| | | CAGCAACAGCAGCATGCCGGGCTACTAGGAAAGGGTACACCAAACGCTCTACAGCAGCAG |
| | | CTACACATGAACCAGCTCACCGGGATACCTCCACCGGGACTCATGAACAACAGCGATGTA |
| | | CACACTTCCAGCAACAACAACTCGCGCCAGTTGCTCGACCAATTGGCTAATGGGAACGCG |
| | | AACATGCTCAATATGAACATGGATAACAACAATAACAACAATAACAACAACAACAACAAC |
| | | AACAACAACGGCGGTGGAAGCGGGGTGATGATGAATGCCTCCACAGCCGCAGTAAATTCT |
| | | ATAGGGATGGTTCCTACCGTTGGTACGCCTGTGAACATCAATGTGAACGCCAGTAACCCT |
| | | TTATTACACCCGCACTTGGATGACCCTTCTTTACTAAACAATCCGATCTGGAAGCTTCAA |
| | | TTGCATCTGGCTGCAGTGTCCGCACAATCTCTGGGGCAACCCAACATTTATGCTAGACAA |
| | | AACGCTATGAAGAAGTATTTGGCTACACAACAGGCTCAGCAAGCTCAGCAACAAGCGCAG |
| | | CAACAGGCACAGCAGCAGGTCCCGGGCCCATTTGGCCCCGGACCTCAGGCTGCACCACCA |
| | | GCTTTGCAGCCCACCGATTTCCAGCAATCTCACATTGCAGAAGCCTCCAAATCACTGGTA |
| | | GACTGCACAAAGCAAGCCTTGATGGAAATGGCCGACACTCTCACCGACAGCAAGACAGCA |
| | | AAGAAACAACAACCTACGGGAGATAGCACTCCCTCAGGCACGGCAACTAACAGTGCAGTT |
| | | TCTACACCATTGACTCCCAAGATAGAGCTGTTTGCTAATGGCAAGGACGAAGCCAACCAG |
| | | GCGCTCTTACAACACAAGAAACTGTCTCAGTACAGCATCGACGAAGATGACGACATTGAA |
| | | AACAGAATGGTCATGCCCAAGGACTCGAAATACGACGACCAATTATGGCACGCGCTAGAT |
| | | TTGTCCAACTTGCAAATCTTCAATATCAGCGCCAACATCTTCAAGTACGATTTTCTAACG |
| | | AGACTATATTTGAATGGCAATAGCCTCACGGAACTGCCAGCGGAGATCAAGAACCTAAGC |
| | | AACCTACGCGTTTTGGACCTGTCGCATAATAGGTTAACATCTCTACCAGCGGAACTAGGC |
| | | TCATGTTTCCAATTGAAATACTTCTACTTTTTTGATAACATGGTCACCACATTACCATGG |
| | | GAGTTTGGGAACCTGTGTAACCTTCAGTTTCTTGGTGTAGAAGGAAACCCCTTAGAAAAA |
| | | CAGTTTTTAAAGATCCTTACAGAAAAATCTGTCACGGGATTGATTTTCTACCTAAGGGAC |
| | | AACAGACCAGAGATTCCCTTACCGCATGAACGTAGGTTCATCGAAATCAATACCGATGGG |
| | | GAACCACAGAGGGAGTACGATTCTTTGCAGCAATCCACTGAGCATCTGGCCACCGATTTA |
| | | GCCAAGAGGACGTTCACCGTCTTATCCTACAACACCTTATGTCAACACTATGCCACCCCA |
| | | AAAATGTACCGTTACACACCGTCGTGGGCGTTAAGTTGGGATTACAGGCGCAATAAATTA |
| | | AAGGAGCAGATTCTCTCGTACGACAGTGATCTGTTGTGTTTACAAGAAGTGGAGTCTAAG |
| | | ACTTTTGAAGAGTATTGGGTGCCCCTATTGGACAAGCACGGTTATACAGGCATCTTCCAT |
| | | GCAAAGGCAAGAGCCAAGACCATGCATTCCAAGGACTCCAAGAAAGTGGACGGGTGTTGC |
| | | ATTTTTTTCAAAAGGGACCAATTCAAGTTGATCACCAAAGACGCCATGGATTTCAGCGGT |
| | | GCTTGGATGAAACACAAGAAGTTCCAAAGAACTGAAGATTATTTAAACCGTGCAATGAAC |
| | | AAAGACAACGTTGCACTGTTCTTAAAGCTACAACACATTCCTAGTGGCGACACCATATGG |
| | | GCGGTCACCACGCATTTGCACTGGGATCCAAAATTTAATGATGTCAAGACATTCCAAGTA |
| | | GGTGTCCTGTTAGATCATCTGGAAACGCTGCTAAAGGAGGAGACATCGCACAATTTTAGA |
| | | CAGGACATTAAGAAATTTCCTGTGCTCATTTGTGGTGACTTCAATTCATACATCAACTCC |
| | | GCCGTATACGAATTGATAAATACAGGCCGTGTCCAAATACATCAAGAGGGAAATGGTAGA |
| | | GATTTCGGTTACATGTCGGAGAAAAATTTCTCACATAACTTGGCTCTCAAATCAAGTTAT |
| | | AATTGTATCGGAGAACTACCATTCACCAATTTCACACCGTCATTCACAGATGTTATCGAC |
| | | TATATATGGTTTTCTACACATGCTCTAAGGGTGCGTGGGCTATTGGGTGAAGTGGACCCT |
| | | GAATACGTGAGTAAGTTTATCGGGTTCCCCAACGACAAATTCCCCAGTGACCATATACCA |
| | | TTGTTAGCAAGATTTGAATTTATGAAGACAAACACAGGCAGTAAGAAAGTATAA |
| YAL047C* | SPC72 | ATGGTACGTCGATGGATTCCTAGTGGCAGGCATCTTCGCAATAATGACAACACTGGTGAT |
| | | GATGACGACAGCGAGTTCACAAACTCGATGGATTCTGGGATGTCCATACCATCACTCAGG |
| | | GACTCCATGACCACGAGGTCATCTCATAACGATCCCATCAAGCCTGCTCTGATGAACGAT |
| | | TCCAACAAAGTCAAAAATTTGGAGAAGGAGTTGACTAATGCCAAAATCAAGATTCAAGTA |
| | | CTCTATGAATACATTCGCAGAATCCCTAATAAAGACGGCAATGCACCATCGCTGGGCAAT |
| | | GACACTGATTTTAGAAATTCGATTATCGAGGGTCTAAATCTTGAAATAAACAAATTGAAA |
| | | CAGGATTTAAAGGCGAAGGAAGTCGAGTACCAAGATACGCTACAATTCGTTCAAGAGAAT |
| | | TTAGAAAATTCGGAAAGTATCGTGAATACGATCAATCACCTATTGTCCTTTATATTGACG |
| | | CATTTTAATGAGCAAGATGAAAATGCCCATCTTCTTGATAAAGAAGAGAGGGAGACCCTG |
| | | GAGGAAACTTTAGAGCTGAGCTCGGATTATGTTCTTGAAAAAATGGATACTTTGTCCAAG |
| | | TTCATCATCCAATTCTTGCAGGACTTTTTGCATTCCAAAAGTCGAGCGGAATCAAAGCAG |
| | | GATAAGGAAGAATTTCTTTCACTGGCCCAGTCCTCACCAGCAGGATCACAGTTAGAAAGT |
| | | AGGGACTCACCATCAAGTAAAGAGGAGAATACTGATGGTGGATACCAGAATGACGAAATT |
| | | CATGACAGTAACAACCACATTGATACAGAAAATGTAATGGCAAACAGTACTTCCTTACCA |
| | | ATTTCAGCCGTCGAGTCTCGTTTTGAGAAAACCTTAGACACTCAATTAGAGATTGTCATA |
| | | GAAATTTTGCACAAAGAATATGATCAATTTATAAATTCCATTAGATTGAAATTCGAGAAA |
| | | TCGCAAAAATTAGAAAAAATAATAGCGTCCAAACTAAATGAGCAGTCTCATCTACTAGAT |
| | | TCCTTAGAATTGGAAGAAAATTCTAGTTCCGTTATAGAAAAACAAGATCATTTGATTTCC |
| | | CAACTGAAGGAAAAGATCGAATCGCAATCTGTTTTGATAAACAATTTGGAAAAATTGAAG |
| | | GAAGACATCATTAAAATGAAACAAAATGAAAAAGTTTTAACCAAAGAACTGGAAACTCAG |
| | | ACCAAAATCAATAAACTAAAGGAAAATAATTGGGACAGCTACATCAATGACTTGGAAAAA |
| | | CAAATCAATGACCTTCAAATCGATAAATCAGAGGAATTCCACGTAATACAAAATCAGTTA |
| | | GACAAATTAGACTTGGAGAATTACCAATTAAAAAATCAGTTAAATACTTTGGACAACCAA |
| | | AAGTTAATACTATCCCAATATGAAAGCAATTTTATCAAATTTAATCAGAACCTTTTACTA |
| | | CACCTTGACAGTATTTTCAATATTCTGCAGAAAATCTTACAAGAAAGTTCTATTGCCCAA |
| | | TTTGACAGGAAAATGAAATCCATAAAATCTGTACCAAATGCGTTGAAAAACTTGAATTTG |
| | | ATTCAGCCCAAACTAGAATCGCTCTACACTTTTATAGAGACTGCATTAGAGTCCATAATA |
| | | AACTCATATATCTCTTCACTGATTTCCATGGAAACCCCAGAGCAGCCACATCAACAGGGC |
| | | AATGAATTAACGGCAACTCCAAATAAAGAACTGACCTTACGAATAGAGGAGTTACAAAGA |
| | | AGATGGATATCCGAAAGAGAAAGACGGAAGCTCGATGCTAACGCTTCAGAAGCTCGAATC |
| | | AAGGCTTTAGAACAGGAAAATGAGTCATTGAGATCGAAACTTTTCAACCTATCAATCAAC |
| | | AATCCCTAA |
| YBL058W | SHP1 | ATGGCGGAAATACCTGATGAAACCATCCAGCAGTTCATGGCATTGACCAATGTGTCGCAT |
| | | AACATAGCCGTTCAATATCTCTCTGAATTTGGAGATTTAAATGAAGCACTAAATTCCTAT |
| | | TATGCTTCTCAAACGGATGACCAAAAGGATAGAAGAGAGGAAGCACATTGGAACAGACAG |
| | | CAGGAGAAGGCCCTCAAGCAAGAAGCCTTCTCCACCAACTCTTCGAATAAAGCCATAAAT |
| | | ACGGAGCACGTTGGTGGGTTATGTCCAAAACCAGGATCCTCACAAGGTAGCAACGAGTAC |
| | | TTGAAAAGGAAAGGTTCTACCTCTCCTGAACCAACCAAGGGTAGTAGCCGCTCTGGAAGT |
| | | GGTAACAACTCCAGGTTTATGAGCTTTTCGGATATGGTAAGAGGTCAAGCTGATGATGAC |
| | | GATGAAGATCAACCGAGAAATACTTTTGCTGGTGGTGAAACATCCGGCTTAGAGGTTACA |
| | | GATCCTTCAGATCCTAATTCATTACTGAAGGATTTGCTGGAAAAAGCGAGAAGGGGTGGT |
| | | CAAATGGGCGCTGAAAACGGATTCCGTGATGACGAAGACCATGAAATGGGTGCCAATAGG |
| | | TTTACTGGAAGAGGTTTTAGATTAGGGTCAACCATCGACGCAGCAGATGAAGTCGTAGAA |
| | | GACAACACTTCACAATCACAACGTAGACCAGAAAAAGTCACAAGAGAAATTACATTTTGG |
| | | AAGGAAGGTTTTCAAGTGGCCGATGGTCCGCTTTATCGCTATGATGATCCTGCGAACAGT |
| | | TTCTATTTGAGCGAGTTAAATCAAGGGAGGGCTCCATTAAAGCTCTTAGATGTGCAATTT |
| | | GGACAAGAAGTTGAAGTTAATGTATATAAAAAATTAGATGAGTCTTATAAAGCTCCGACG |
| | | AGAAAACTGGGCGGTTTTTCAGGCCAGGGCCAAAGACTAGGATCTCCTATCCCGGGTGAA |
| | | TCGTCACCTGCGGAGGTTCCAAAGAATGAGACACCCGCTGCTCAGGAACAACCCATGCCG |
| | | GACAATGAGCCAAAACAAGGCGACACCTCCATCCAAATTAGATACGCAAATGGCAAAAGA |
| | | GAAGTTTTGCACTGCAATTCCACAGATACAGTAAAGTTTTTGTATGAGCATGTGACATCA |
| | | AATGCGAACACTGACCCATCGAGGAATTTCACCTTGAATTATGCCTTTCCTATCAAACCA |
| | | ATAAGCAACGATGAGACAACATTGAAGGACGCTGATCTGCTGAACTCCGTTGTCGTGCAA |
| | | AGATGGGCATGA |
| YBL071W-A | KTI11 | ATGTCAACATATGACGAAATCGAAATCGAAGATATGACGTTTGAGCCTGAAAATCAAATG |
| | | TTCACCTATCCTTGTCCCTGTGGAGATAGGTTTCAAATATATCTGGATGACATGTTTGAG |
| | | GGCGAAAAAGTTGCTGTTTGTCCCAGCTGCTCACTGATGATCGATGTAGTTTTCGATAAA |
| | | GAAGACTTGGCTGAGTACTACGAAGAGGCAGGCATCCACCCCCCTGAGCCTATTGCCGCT |
| | | GCTGCCTAA |
| YBL093C* | ROX3 | ATGGCTTCTAGAGTGGACGAAACTACAGTCCCCTCATACTACTATTACGTGGATCCGGAA |
| | | ACTACATATACGTACCAACAACCAAATCCTCTACAGGACTTGATATCGGTGTATGGCTTG |
| | | GATGACATCTCCAGGCAAGTGGCAAGAACAAATTTGGACGGCACTAAAGCCGTGAAGCTA |
| | | AGAAAATCTTACAAGAACCAGATAGCAGATCTTTCAGGTAAATTCTCCACCATACCGACC |
| | | AGAGAAAATGGTAAAGGTGGTCAAATAGCACATATTCTTTTCCAAAATAACCCAGACATG |
| | | ATGATACAACCACCTCAGCAGGGTCAAAACATGTCAGAGCAACAATGGCGCGAACAGCTG |
| | | CGCAATAGAGACATAGCATTGTTTCAGCCTCCAAATTTCGATTGGGACCTTTGCTCTTCG |
| | | GTACTATCGCAGTTTGAAAGGTCATATCCAAGCGAGTTCGCAAACCAGAACCAAGGGGGA |
| | | GCCCAAGCGCCGTTTGATATAGACGACTTGGCGTTTGATCTAGACGGTACAGGAAAAAGC |
| | | CAATCCGGCTCAAATTCAGGTAACAATAGTAAGAAAAGGAAGAACAAATCTAGTGGAAGT |
| | | TCGATGGCTACACCAACACATAGTGACAGTCATGAGGATATGAAAAGAAGGAGGCTGGAG |
| | | TAG |
| YBR023C* | CHS3 | ATGACCGGCTTGAATGGAGATGATCCTGATGACTACTATCTGAACCTTAATCAAGATGAA |
| | | GAGTCTCTACTTAGGTCAAGACACAGTGTCGGCTCAGGAGCACCTCATAGACAAGGCTCT |
| | | TTAGTGCGGCCCGAAAGAAGCCGACTGAACAATCCTGATAATCCACATTTTTATTATGCG |
| | | CAGAAAACGCAGGAGCAGATGAATCACCTGGATGTTTTACCATCAAGTACCGGTGTAAAC |
| | | CCAAATGCAACTCGTCGGAGTGGCTCCCTGCGCTCCAAAGGCTCAGTGAGAAGCAAATTT |
| | | AGTGGCCGCGAAACGGATAGCTATCTTTTACAAGATATGAATACTACTGACAAGAAGGCT |
| | | TCCGTTAAAATAAGTGATGAAGGTGTTGCGGAAGACGAATTTGATAAAGATGGTGATGTG |
| | | GACAATTTCGAAGAAAGCTCCACGCAGCCCATAAATAAGTCTATCAAACCATTAAGAAAG |
| | | GAAACGAATGATACATTGTCATTTTGGCAGATGTACTGTTATTTCATTACGTTTTGGGCA |
| | | CCTGCTCCAATTCTTGCTTTCTGCGGGATGCCAAAGAAGGAAAGACAAATGGCGTGGAGA |
| | | GAAAAGGTTGCTTTAATTTCTGTCATCTTGTACATTGGTGCGATTGTGGCTTTCCTGACT |
| | | TTTGGTTTCACTAAAACCGTTTGTAGTAGTTCGAAACTACGTTTGAAAAACAACGAAGTA |
| | | TCAACAGAATTTGTCGTAATTAACGGTAAGGCTTATGAATTGGATACTTCCTCGCGTTCC |
| | | GGTATACAAGACGTTGAAGTAGATTCAGACACCCTTTATGGGCCCTGGTCAGATGCTGGT |
| | | AAAGATGCTTCGTTCTTGTTTCAAAATGTGAATGGTAACTGTCATAACCTTATAACTCCA |
| | | AAGAGTAATTCTTCCATTCCCCATGACGATGATAATAATTTAGCATGGTATTTTCCTTGT |
| | | AAGTTAAAGAATCAAGATGGCTCTTCGAAGCCAAACTTCACAGTTGAAAATTACGCAGGA |
| | | TGGAACTGTCATACGTCTAAAGAAGATAGGGACGCATTTTACGGTTTAAAGTCGAAAGCT |
| | | GATGTTTACTTCACTTGGGATGGTATAAAGAACTCTTCTAGAAACTTGATTGTTTATAAT |
| | | GGCGACGTTTTGGATTTAGATCTTCTTGATTGGTTGGAAAAGGATGACGTTGACTATCCC |
| | | GTTGTATTCGATGACTTGAAGACTTCAAATTTACAAGGTTATGATCTTTCGTTGGTTTTG |
| | | TCAAATGGGCATGAAAGAAAAATTGCGAGATGTTTGAGCGAAATTATTAAAGTTGGTGAA |
| | | GTAGACTCCAAAACCGTCGGTTGTATTGCCTCTGATGTCGTTTTGTATGTTTCTCTGGTA |
| | | TTTATTCTTTCAGTGGTGATAATTAAATTCATAATTGCCTGCTACTTCCGTTGGACTGTA |
| | | GCTAGGAAACAAGGTGCATATATCGTGGACAATAAAACAATGGATAAACACACAAACGAT |
| | | ATCGAGGATTGGTCTAATAATATTCAAACAAAAGCTCCTCTAAAGGAAGTAGATCCTCAT |
| | | TTGAGGCCAAAGAAATACTCAAAAAAGTCGTTGGGACACAAGCGTGCTTCAACCTTTGAC |
| | | TTGCTGAAAAAACACAGCTCCAAAATGTTTCAATTTAACGAATCTGTGATAGATCTAGAC |
| | | ACCTCCATGAGCAGTTCACTACAATCTTCTGGTTCATACAGAGGAATGACAACAATGACC |
| | | ACTCAAAATGCTTGGAAACTCTCGAATGAAAACAAAGCTGTACATTCCCGTAATCCATCT |
| | | ACTTTGTTGCCTACATCCTCGATGTTTTGGAATAAAGCGACTTCCTCTCCTGTACCAGGA |
| | | TCATCGCTGATTCAGAGTCTTGATTCGACGATTATACATCCCGATATCGTTCAACAACCA |
| | | CCACTGGATTTTATGCCATACGGGTTCCCATTGATTCATACTATCTGTTTTGTTACTTGT |
| | | TATTCTGAGGATGAAGAGGGTTTAAGAACCACTTTAGACTCTCTTTCTACCACAGATTAT |
| | | CCAAATTCCCATAAACTACTGATGGTTGTTTGTGATGGTTTAATTAAGGGCTCGGGCAAC |
| | | GATAAGACTACTCCAGAGATAGCGTTAGGAATGATGGACGACTTTGTCACCCCACCTGAT |
| | | GAAGTTAAACCTTACTCCTATGTGGCAGTGGCATCAGGCTCTAAAAGACACAATATGGCC |
| | | AAGATATATGCGGGTTTTTACAAATATGACGATTCTACAATTCCACCAGAAAATCAACAA |
| | | CGTGTCCCAATCATTACAATTGTGAAGTGCGGTACTCCTGCAGAGCAGGGGGCCGCCAAA |
| | | CCCGGTAACAGAGGTAAGCGTGATTCTCAAATTATTCTGATGTCCTTTTTAGAAAAAATA |
| | | ACATTTGATGAAAGAATGACTCAATTGGAATTTCAGCTTTTAAAAAATATTTGGCAGATT |
| | | ACGGGGCTAATGGCAGACTTCTACGAAACGGTACTTATGGTTGATGCTGATACTAAAGTC |
| | | TTTCCCGATGCTTTAACTCATATGGTCGCTGAAATGGTTAAAGATCCTTTGATTATGGGT |
| | | CTTTGTGGTGAGACCAAGATCGCTAATAAGGCACAATCTTGGGTAACTGCAATTCAAGTG |
| | | TTTGAGTACTATATTTCGCATCATCAGGCTAAAGCTTTTGAATCTGTCTTCGGTTCGGTA |
| | | ACTTGTTTGCCGGGATGTTTCTCAATGTATCGTATAAAATCTCCTAAAGGTTCAGATGGT |
| | | TATTGGGTACCTGTATTGGCAAATCCAGATATTGTTGAAAGATATTCGGATAATGTTACA |
| | | AACACTTTGCATAAGAAGAACTTATTATTACTTGGTGAAGATAGATTTTTATCTTCATTA |
| | | ATGTTAAAGACTTTCCCTAAGAGAAAGCAAGTATTTGTTCCAAAAGCTGCTTGTAAAACT |
| | | ATTGCCCCTGATAAATTCAAAGTCTTACTTTCCCAGCGTCGAAGATGGATTAATTCTACG |
| | | GTACATAACCTTTTTGAATTAGTTCTAATCAGAGACTTATGTGGCACTTTCTGTTTTTCC |
| | | ATGCAATTTGTGATTGGTATTGAATTGATTGGTACTATGGTACTGCCGTTAGCCATTTGC |
| | | TTTACTATTTATGTCATTATTTTTGCCATTGTATCAAAACCTACACCCGTAATCACTTTA |
| | | GTTTTACTGGCAATTATTCTTGGTCTGCCCGGCTTAATTGTTGTTATAACTGCTACGAGA |
| | | TGGTCGTACCTATGGTGGATGTGCGTATATATTTGTGCTTTGCCTATTTGGAATTTCGTA |
| | | CTACCTTCATATGCGTACTGGAAATTTGATGACTTCTCATGGGGTGATACGAGAACTATT |
| | | GCGGGAGGTAATAAAAAGGCACAAGACGAGAATGAAGGTGAATTTGATCACTCAAAGATT |
| | | AAAATGAGGACATGGAGGGAATTTGAAAGGGAAGATATTCTCAATCGGAAGGAGGAAAGT |
| | | GACTCCTTCGTTGCATAG |
| YBR081C* | SPT7 | ATGACTGAAAGAATACCAATAAAGAATTATCAAAGAACAAATGCCAAAGCTTTACTTAAA |
| | | TTGACTGAAAAACTTTTTAACAAGAACTTTTTTGATCTCTATTTAACCTCTCAGCAATTG |
| | | GTCGTTCTTGAATACCTGCTGTCGATTTCAAGTGAAGAAGACAAACTGAAAGCATGGGAC |
| | | TATTTCTTAAAGGGAAACATAGCATTAAATGTCGAAAAATCATTTCCATTAACCCAAGAA |
| | | GAAGAACATCACGGAGCGGTCTCTCCTGCCGTTGACACACGATCAGATGATGTATCATCA |
| | | CAAACAATTAAGGACAATAACAATACTAATACCAACACCAGTATCAGCAATGAAAATCAT |
| | | GTTGAAAATGAAATTGAAGATAAAGGCGATAACGCAATAGCAAATGAAGATAATTTTGTG |
| | | AATAATGACGAAAGTGATAATGTTGAAGAAGACTTATTCAAATTAGATCTAGAGGACTTG |
| | | AAGCAGCAAATAAGCGGAACAAGGTTTATTGGAAACTTATCCTTGAAAATCAGATACGTC |
| | | TTGTGGCAGTGCGCCATAGATTATATATACTGTGATCGTAATGAGTTTGGTGATGAAAAT |
| | | GATACAGAATACACCCTATTAGATGTTGAAGAGAAGGAGGAAGAGGAAATTGGTAAAAAT |
| | | GAGAAGCCACAAAACAAAGAAGGTATTTCGAAGTTCGCCGAGGATGAAGATTACGACGAT |
| | | GAAGACGAGAACTATGATGAAGACAGTACAGACGTAAAAAATGTCGATGATCCTCCAAAA |
| | | AATCTCGATTCTATTTCCTCTTCTAATATCGAAATTGACGATGAACGACGCTTGGTGCTA |
| | | AATATCTCAATATCAAAAGAAACACTGTCAAAGTTAAAAACAAATAATGTAGAAGAAATT |
| | | ATGGGAAATTGGAACAAAATTTACCACAGTTTTGAATACGATAAAGAAACTATGATAAAG |
| | | CGATTAAAACTTGAAGAAAGCGATAAAATGATAGAGAAAGGAAAGAAGAAACGAAGTCGA |
| | | AGTGATTTAGAAGCAGCTACCGATGAACAAGATCGCGAAAATACAAATGATGAGCCAGAT |
| | | ACTAATCAAAAATTGCCCACTCCTGAAGGTTCAACATTCAGCGATACTGGGAACAAGCGC |
| | | CCCAAACAAAGTAATTTAGATTTAACAGTCAATCTAGGCATCGAAAATTTATCATTAAAG |
| | | CACCTTCTATCATCTATCCAGCAAAAAAAATCCCAATTAGGAATATCAGATTACGAATTA |
| | | AAACATCTGATTATGGATGTCAGAAAAAATCGGTCAAAATGGACATCGGATGAAAGAATT |
| | | GGGCAAGAGGAATTATACGAAGCCTGTGAAAAGGTTGTTTTGGAACTTAGAAACTACACT |
| | | GAGCATTCTACACCATTTCTGAATAAAGTGAGCAAAAGAGAAGCCCCCAATTATCATCAA |
| | | ATCATCAAAAAGTCCATGGACCTGAATACTGTTTTAAAAAAACTGAAAAGCTTTCAATAT |
| | | GACTCCAAACAAGAATTTGTAGACGATATTATGCTAATATGGAAAAATTGTTTGACCTAT |
| | | AATTCAGATCCTTCACATTTTTTGAGAGGGCATGCTATTGCTATGCAGAAGAAATCTCTT |
| | | CAGTTGATTCGCATGATTCCAAATATCACAATCCGAAACAGGGCTGATTTAGAAAAGGAA |
| | | ATTGAAGATATGGAAAAAGACAAAGACTACGAATTAGATGAGGAAGAGGAAGTTGCTGGT |
| | | TCTGGAAGAAAAGGATTGAATATGGGAGCTCATATGTTGGCCAAAGAGAATGGCAAGGTG |
| | | TCAGAAAAAGATAGCTCTAAAACCGTCAAGGATGAAGCACCAACCAATGATGACAAACTA |
| | | ACTTCTGTCATCCCTGAGGGGGAAAAAGAGAAAGATAAAACTGCTTCATCTACTGTAACG |
| | | GTACACGAAAATGTAAATAAGAACGAAATAAAAGAAAATGGGAAAAATGAAGAGCAAGAT |
| | | ATGGTTGAGGAAAGTAGTAAGACTGAGGATTCATCAAAAGATGCTGATGCTGCCAAAAAG |
| | | GATACGGAAGACGGACTACAAGATAAAACTGCAGAAAATAAGGAGGCTGGGGAAAATAAT |
| | | GAAGAGGAAGAGGATGATGATGACGAAGATGAAGACGAAGACATGGTCGACTCCCAATCT |
| | | TATTTACTTGAAAAGGATGACGATAGAGACGATTTGGAAATATCCGTGTGGAAAACTGTA |
| | | ACTGCCAAAGTTCGTGCGGAAATTTGCTTAAAAAGAACTGAATATTTTAAAAATGGAAAA |
| | | TTAAATAGTGATTCAGAGGCGTTTTTGAAAAACCCACAAAGAATGAAAAGGTTCGACCAG |
| | | CTTTTTCTTGAATATAAAGAGCAGAAAGCTTTAGAATCATATCGTCAAAAAATAGAGCAA |
| | | AATTCCATTATGAAAAATGGCTTTGGAACAGTACTAAAACAGGAAGACGATGACCAATTG |
| | | CAGTTTCATAATGATCACTCTTTAAATGGAAATGAAGCTTTTGAAAAGCAACCCAATGAT |
| | | ATTGAGTTAGATGATACCAGATTCCTACAGGAATATGATATTAGTAACGCCATTCCTGAC |
| | | ATAGTATACGAGGGAGTAAATACTAAAACATTAGACAAGATGGAAGACGCTTCCGTGGAC |
| | | CGCATGCTTCAAAATGGTATCAACAAACAAAGCAGATTTCTGGCTAACAAGGATTTAGGA |
| | | CTAACACCTAAAATGAACCAAAATATCACACTGATTCAGCAAATTAGGCACATATGCCAT |
| | | AAAATATCCCTGATCAGAATGTTACAGAGCCCTTTATCGGCTCAAAACTCCAGAAGCAAT |
| | | CCCAACGCTTTCCTTAACAACCACATTTATAATTACACTATTATTGATGACTCACTCGAT |
| | | ATTGATCCGGTGTCACAGCTTCCAACGCATGATTACAAAAACAACAGGGAGCTGATATGG |
| | | AAATTCATGCATAAGAACATATCTAAGGTTGCTATGGCCAATGGGTTTGAAACTGCCCAT |
| | | CCATCAGCAATAAACATGCTTACTGAAATCGCCGGGGATTACCTATCTAATCTGATAAAG |
| | | ACTTTGAAGCTTCATCATGAAACTAACTCCTTAAATAGAGGAACAAATGTGGAAATGCTG |
| | | CAAACAACACTGTTGGAAAACGGTATCAACAGGCCAGACGATCTATTTTCCTATGTTGAA |
| | | TCTGAATTTGGTAAAAAAACTAAGAAACTTCAGGACATCAAACAGAAACTAGAAAGCTTT |
| | | TTGAGAGCCTTATTAAGGCCAACTTTGCAGGAGTTGTCCGAGAGAAACTTTGAAGACGAG |
| | | AGCCAAAGCTTTTTTACAGGTGACTTTGCCAGCGAATTGACTGGTGAAGACTTCTTTGGT |
| | | TTTAGAGAGCTTGGATTAGAAAAGGAGTTTGGAGTTTTGAGTTCATCTGTTCCATTACAG |
| | | TTACTGACTACTCAGTTTCAAACTGTTGACGGGGAAACCAAAGTGCAGGCCAAAAAGATC |
| | | CAACCGGAAGAATCAGACAGCATTGTGTATAAGAAAATTACAAAAGGTATGCTGGATGCT |
| | | GGTTCATTCTGGAATACTCTACTTCCCCTATTACAAAAAGATTATGAACGTTCCAAGGCC |
| | | TATATAGCAAAGCAAAGCAAGTCATCTGCAAATGATAAAACCTCAATGACTTCCACAGAA |
| | | GACAATTCTTTCGCTTTACTAGAAGAGGATCAGTTTGTCTCAAAGAAAACCGCAACGAAG |
| | | GCAAGATTACCTCCTACTGGTAAGATAAGTACCACATACAAAAAGAAACCGATCGCAAGC |
| | | GCGTTTATACTTCCAGAAGAAGACTTGGAAAACGACGTAAAAGCGGATCCAACAACAACT |
| | | GTAAACGCCAAAGTGGGTGCAGAAAATGATGGAGATTCTTCCTTATTTTTGCGAACGCCT |
| | | CAACCTTTAGATCCTTTGGATATGGATGATGCTTTTGATGATACCAATATGGGCAGCAAT |
| | | AGTTCATTTAGCTTGAGCCTTCCTCGCCTTAATCAATAA |
| YBR112C* | CYC8 | ATGAATCCGGGCGGTGAACAAACAATAATGGAACAACCCGCTCAACAGCAACAACAACAG |
| | | CAACAACAACAGCAGCAACAGCAACAGCAGGCAGCAGTTCCTCAGCAGCCACTCGACCCA |
| | | TTAACACAATCAACTGCGGAAACTTGGCTCTCCATTGCTTCTTTGGCAGAAACCCTTGGT |
| | | GATGGCGACAGGGCCGCAATGGCATATGACGCCACTTTACAGTTCAATCCCTCATCTGCA |
| | | AAGGCTTTAACATCTTTGGCTCACTTGTACCGTTCCAGAGACATGTTCCAAAGAGCTGCA |
| | | GAATTATATGAAAGAGCACTTTTGGTAAATCCCGAACTATCAGATGTGTGGGCTACTTTA |
| | | GGTCATTGTTATCTGATGCTGGATGATCTGCAAAGAGCTTACAATGCCTATCAACAGGCT |
| | | CTCTACCACCTCAGTAATCCCAACGTACCGAAATTATGGCATGGAATCGGCATTCTTTAT |
| | | GACAGATATGGTTCGCTCGACTATGCCGAAGAAGCTTTTGCCAAAGTTTTGGAATTGGAC |
| | | CCTCATTTTGAAAAGGCAAACGAAATTTACTTCAGACTAGGTATTATTTATAAACATCAG |
| | | GGTAAATGGTCTCAAGCTTTGGAATGCTTCAGATACATTCTCCCTCAACCTCCTGCTCCC |
| | | TTGCAGGAGTGGGACATATGGTTTCAGTTGGGTAGTGTTTTGGAGAGTATGGGAGAGTGG |
| | | CAAGGTGCGAAGGAAGCCTACGAGCATGTCTTGGCTCAAAATCAACATCATGCCAAAGTA |
| | | TTACAACAATTAGGTTGTCTTTACGGTATGAGTAACGTACAATTTTATGACCCTCAAAAG |
| | | GCATTGGATTATCTTCTAAAGTCGTTAGAAGCAGATCCCTCCGATGCCACTACATGGTAC |
| | | CATCTCGGTAGAGTGCATATGATTAGAACAGATTATACTGCCGCATATGATGCTTTCCAA |
| | | CAAGCTGTTAATAGAGATTCAAGAAACCCTATCTTTTGGTGCTCAATCGGTGTTTTATAT |
| | | TACCAAATTTCTCAATACAGAGACGCCTTAGACGCGTACACAAGAGCCATAAGATTAAAT |
| | | CCTTATATTAGTGAAGTTTGGTACGATCTAGGTACTCTTTACGAAACTTGTAACAACCAA |
| | | TTATCTGACGCCCTTGATGCGTATAAGCAAGCTGCAAGACTGGACGTAAATAATGTTCAC |
| | | ATAAGAGAAAGATTAGAAGCTTTAACAAAGCAGTTAGAAAACCCAGGCAATATAAACAAA |
| | | TCGAACGGTGCGCCAACGAATGCCTCTCCTGCCCCACCTCCTGTGATTTTACAACCTACC |
| | | TTACAACCTAATGATCAAGGAAATCCTTTGAACACTAGAATTTCAGCCCAATCTGCCAAT |
| | | GCTACTGCTTCAATGGTACAACAACAGCATCCTGCTCAACAAACGCCTATTAACTCTTCT |
| | | GCAACAATGTACAGTAATGGAGCTTCCCCTCAATTACAAGCTCAAGCTCAAGCTCAAGCT |
| | | CAAGCACAAGCTCAAGCACAAGCACAAGCTCAAGCACAAGCACAAGCACAAGCGCAAGCA |
| | | CAAGCACAAGCACAGGCGCAAGCACAGGCACAAGCACAAGCACAAGCACATGCACAAGCG |
| | | CAAGCACAAGCACAAGCACAGGCACAAGCACAAGCACAGGCGCAGGCACAACAACAACAA |
| | | CAACAACAGCAACAACAACAACAACAACAACAACAACAACAACAACAACAACAACAACAA |
| | | CAACAACAACAGCAGCAGCAATTACAGCCCCTACCAAGACAACAGCTGCAGCAAAAGGGA |
| | | GTTTCTGTGCAAATGTTAAATCCTCAACAAGGGCAACCATATATCACACAGCCAACAGTC |
| | | ATACAAGCTCACCAACTGCAACCATTTTCTACACAAGCTATGGAACATCCGCAAAGCTCT |
| | | CAACTGCCACCTCAACAGCAACAACTACAATCTGTTCAACATCCACAACAACTTCAAGGC |
| | | CAGCCTCAAGCCCAAGCTCCCCAACCTTTAATCCAGCATAACGTGGAACAGAACGTTTTA |
| | | CCTCAAAAGAGATACATGGAAGGTGCAATCCACACTTTAGTAGATGCCGCCGTATCCAGT |
| | | AGCACCCACACAGAGAATAACACAAAGTCTCCTCGTCAACCAACCCATGCCATTCCAACG |
| | | CAAGCTCCCGCAACAGGAATAACGAACGCTGAACCACAGGTAAAGAAGCAAAAGTTGAAC |
| | | TCTCCAAATTCAAACATCAACAAATTAGTAAATACTGCTACTTCCATTGAAGAAAATGCA |
| | | AAATCTGAGGTGAGCAACCAATCGCCAGCAGTAGTGGAGTCTAATACCAATAATACTTCA |
| | | CAAGAAGAAAAACCTGTAAAAGCAAACTCAATACCTTCAGTAATTGGCGCACAGGAACCT |
| | | CCACAGGAAGCTAGTCCTGCTGAAGAAGCTACCAAAGCAGCTTCTGTTTCTCCTTCTACA |
| | | AAACCGCTTAATACGGAACCAGAGTCATCTAGTGTCCAACCAACTGTATCATCAGAAAGT |
| | | TCAACAACAAAAGCAAATGACCAAAGCACTGCTGAGACCATAGAACTTTCTACTGCTACT |
| | | GTTCCTGCAGAAGCAAGCCCTGTAGAAGACGAAGTAAGACAGCATTCTAAAGAGGAAAAC |
| | | GGCACAACTGAAGCATCTGCACCTTCTACTGAAGAGGCGGAGCCAGCAGCTTCCAGAGAT |
| | | GCTGAAAAACAACAAGATGAAACCGCTGCTACAACGATAACTGTAATCAAACCTACTTTG |
| | | GAAACAATGGAAACAGTGAAAGAGGAGGCCAAAATGCGTGAGGAAGAGCAAACATCTCAA |
| | | GAAAAATCCCCACAGGAGAACACACTTCCAAGAGAAAATGTAGTAAGGCAAGTGGAAGAA |
| | | GATGAAAACTACGACGACTAA |
| YBR267W | REI1 | ATGAGTAGCAGTGGTGTTTATACGTGTAATTCATGTGTCCTGACCTTTGATTCAAGCGAC |
| | | GAGCAGCGGGCCCACATGAAGTCCGATTGGCATCGCTACAATTTGAAAAGACGTGTTGCA |
| | | CAATTGCCACCAATATCATTTGAGACATTTGACTCCAAAGTGTCTGCAGCTGCTGCCAGT |
| | | ACTAGTAAGTCTGCTGAAAAGGAGAAACCTGTTACCAAAAAGGAGTTGAAAAGAAGGGAA |
| | | AAGCAAGCATTGCTCGAAAAGAAAAAGAAACTGTTGGAAATTGCCAGGGCTAATATGCTT |
| | | GAAAACATGCAAAAGAGCCAAGAGGGAAATACTCCCGACCTAAGCAAGCTCTCTTTGCAA |
| | | GAAAATGAGGAGAACAAGGAAAAGGAAGAACCTAAGAAGGAGGAGCCTGAACAGTTGACC |
| | | GAGGAAGAAATGGCGGAAAGAGTAATGCAAGAAAACGTACGCAACAGAGTCGATATTCCA |
| | | CTGGAACAATGTCTATTTTGTGAGCACAATAAGCACTTCAAAGATGTTGAAGAAAACCTG |
| | | GAACACATGTTTAGGACCCACGGGTTTTATATCCCAGAACAGAAATATCTAGTCGACAAG |
| | | ATCGGCTTGGTAAAATACATGTCGGAGAAGATTGGTCTTGGGAACATTTGTATTGTTTGT |
| | | AATTACCAGGGGAGAACGTTGACCGCTGTAAGACAGCACATGTTGGCAAAGAGACACTGT |
| | | AAAATTCCCTACGAAAGCGAGGATGAAAGGTTGGAGATATCTGAATTCTACGATTTTACA |
| | | AGCTCATACGCAAACTTTAATAGCAACACAACACCAGACAACGAAGATGACTGGGAAGAC |
| | | GTGGGCAGCGACGAAGCCGGAAGCGACGACGAAGATCTGCCACAAGAGTACTTATATAAC |
| | | GATGGTATAGAGCTGCATCTACCGACAGGCATCAAAGTTGGCCACAGGTCCTTGCAAAGA |
| | | TACTACAAGCAAGACTTAAAGCCCGAGGTGATACTGACCGAAGGCCAAGGTACCCTGGTC |
| | | GCTGCAGAGACGAGATCGTTCTTACCTGCCTTCGACAAAAAGGGCGTGCAGACTCAACAG |
| | | CGTGTTTGGCAAACTGAGAGGTTCGACAAGAAAAGGCTCGATAAGAGAAGTGCCAAGTTC |
| | | GTCAATAACCAACCACACTACAGAGACCAACTTCTGCAGTAA |
| YBR279W | PAF1 | ATGTCCAAAAAACAGGAATATATTGCACCAATAAAATACCAAAACAGCTTGCCTGTGCCA |
| | | CAATTACCTCCTAAACTTCTGGTTTACCCCGAGTCTCCGGAGACGAATGCAGACTCCTCC |
| | | CAACTAATCAATTCACTATACATCAAAACAAACGTCACTAATCTAATTCAACAGGATGAA |
| | | GATTTAGGTATGCCGGTTGATTTGATGAAATTCCCAGGCCTATTGAACAAATTAGATTCT |
| | | AAACTGCTTTACGGCTTTGATAATGTGAAATTGGACAAAGATGATCGAATTTTACTGAGG |
| | | GACCCTAGAATAGATAGACTGACCAAGACTGATATATCAAAGGTTACCTTCTTGAGACGC |
| | | ACCGAATATGTCTCCAATACAATTGCAGCCCATGATAACACATCGTTGAAAAGGAAAAGG |
| | | CGCTTGGATGATGGAGATTCGGATGATGAAAACCTTGATGTTAATCATATAATTAGTAGG |
| | | GTCGAAGGAACATTCAATAAGACGGACAAATGGCAACATCCGGTGAAGAAGGGTGTCAAG |
| | | ATGGTTAAAAAATGGGACCTATTGCCCGACACAGCTTCAATGGACCAGGTGTATTTTATC |
| | | CTAAAATTCATGGGCAGTGCATCATTGGATACTAAAGAAAAAAAATCTTTAAATACAGGA |
| | | ATTTTCAGGCCAGTGGAACTGGAAGAAGACGAATGGATTTCCATGTATGCCACAGATCAT |
| | | AAGGACTCTGCCATTCTGGAAAACGAATTGGAAAAGGGTATGGATGAAATGGATGATGAC |
| | | TCTCATGAAGGTAAAATATACAAGTTTAAAAGGATAAGAGACTACGACATGAAACAAGTG |
| | | GCTGAAAAGCCAATGACAGAGTTAGCTATACGTTTGAATGATAAGGACGGCATAGCTTAC |
| | | TACAAACCCCTACGTTCAAAGATCGAATTAAGACGTAGGAGAGTAAACGACATCATTAAA |
| | | CCTTTGGTGAAGGAGCACGATATAGACCAACTTAATGTTACTTTGAGAAACCCTAGCACT |
| | | AAGGAAGCTAACATAAGGGACAAGTTAAGAATGAAGTTTGACCCCATAAATTTCGCCACT |
| | | GTAGATGAAGAAGACGATGAAGACGAGGAGCAACCAGAAGACGTTAAGAAAGAATCGGAA |
| | | GGCGATTCAAAAACAGAAGGTTCTGAGCAAGAAGGAGAAAATGAGAAGGATGAAGAAATC |
| | | AAACAGGAAAAAGAGAATGAACAAGATGAAGAAAATAAACAAGATGAAAACCGTGCAGCT |
| | | GATACGCCCGAAACTTCAGATGCTGTTCATACTGAACAAAAACCAGAGGAAGAAAAGGAA |
| | | ACTTTACAAGAAGAATAG |
| YBR289W | SNF5 | ATGAATAATCAGCCGCAGGGTACCAACAGCGTTCCAAATAGTATTGGAAATATATTTAGC |
| | | AACATTGGAACTCCATCTTTTAACATGGCGCAAATTCCGCAACAGCTGTATCAGAGCCTC |
| | | ACACCACAACAATTGCAGATGATTCAGCAACGACACCAACAGTTACTGAGGAGTCGTCTA |
| | | CAACAACAACAACAACAACAACAACAAACTTCACCGCCACCGCAAACGCATCAATCTCCA |
| | | CCCCCTCCTCCGCAACAATCTCAACCCATTGCTAATCAATCAGCGACTTCTACCCCTCCT |
| | | CCTCCTCCAGCACCACACAACTTACATCCCCAAATTGGTCAAGTGCCCTTAGCTCCAGCG |
| | | CCTATTAATTTGCCTCCACAAATTGCTCAGTTACCTTTGGCTACACAGCAACAAGTTTTG |
| | | AACAAGTTGAGGCAGCAGGCCATAGCAAAAAATAATCCACAGGTTGTGAATGCAATTACT |
| | | GTTGCACAACAACAAGTGCAACGCCAAATTGAGCAGCAAAAGGGACAGCAAACGGCACAA |
| | | ACTCAGCTAGAACAGCAGAGGCAATTGCTGGTTCAGCAGCAACAGCAGCAGCAACTTAGA |
| | | AACCAAATACAGCGACAACAGCAACAACAGTTTAGGCATCATGTGCAAATACAACAGCAG |
| | | CAACAAAAGCAACAACAACAGCAGCAGCAGCATCAGCAACAACAACAACAACAACAGCAA |
| | | CAGCAGCAACAGCAACAGCAACAGCAGCAGCAACAACAACAGCAACAACAACAACAACAG |
| | | CAGCAGCAGCAGCAGCAGCAGCAAGGACAAATACCGCAATCTCAGCAAGTTCCTCAAGTT |
| | | AGATCCATGAGTGGACAACCTCCCACCAATGTTCAGCCCACTATTGGCCAACTTCCTCAA |
| | | CTTCCAAAATTAAACTTACCCAAGTACCAAACTATTCAATACGATCCACCAGAAACCAAG |
| | | CTACCATATCCAACCTATTGGTCAGACAAAAAAGCAGATACGGATACTTTGTTGTACGAA |
| | | CAAATTATCCAGCGTGATAAAATTAACAAATATTCGCTAATAAGAGAAACCAATGGTTAC |
| | | GATCCGTTTAGCATTTATGGATTTAGTAATAAAGAGTATATTAGTAGACTGTGGCATACA |
| | | CTGAAGTATTATCAAGATTTGAAGAACACTAGAATGAAATCTATCACAAGCACTTCTCAG |
| | | AAGATTCCTTCGGCAAGTATTTGGGGAAATGGTTACTCAGGGTATGGTAATGGGATTACG |
| | | AATACAACTACCAGAGTTATTCCACAAGTAGAAGTTGGAAATAGGAAGCATTACCTAGAG |
| | | GATAAATTAAAAGTCTATAAACAGGCCATGAATGAGACATCGGAACAGTTAGTTCCCATA |
| | | AGATTGGAGTTCGATCAAGATCGTGACAGATTCTTCCTCAGGGACACTTTGTTATGGAAC |
| | | AAAAATGACAAGCTTATTAAAATTGAAGACTTTGTGGACGACATGTTGCGAGATTACCGA |
| | | TTTGAGGACGCTACGAGAGAGCAACACATTGATACTATTTGTCAATCTATACAAGAGCAG |
| | | ATTCAGGAGTTTCAAGGAAATCCATATATAGAGTTGAATCAGGACCGTCTAGGCGGTGAT |
| | | GACTTGAGAATTAGAATCAAGCTGGATATTGTCGTGGGACAAAACCAGTTAATCGATCAA |
| | | TTTGAGTGGGAGATCTCTAATAGTGATAACTGTCCAGAAGAGTTTGCAGAGTCCATGTGT |
| | | CAAGAATTAGAACTACCAGGTGAGTTTGTGACTGCCATTGCTCACTCCATAAGAGAGCAA |
| | | GTTCATATGTATCATAAATCACTGGCACTGTTAGGTTACAATTTTGATGGATCAGCGATA |
| | | GAAGATGATGACATTAGAAGCAGAATGCTCCCAACGATTACTCTTGATGATGTTTATAGG |
| | | CCTGCAGCGGAAAGCAAAATTTTTACTCCAAACCTATTACAGATTTCAGCTGCAGAGTTA |
| | | GAGAGATTGGATAAAGATAAGGACAGAGACACAAGAAGGAAAAGAAGACAAGGTAGATCT |
| | | AATAGACGTGGTATGCTCGCATTGTCCGGCACATCTGCAAGTAATACATCTATGAACGGC |
| | | GTTCACAACACAGTAGCAGCAGGAAATGCTTCATCGTTGCCACCAGGAGAGATTTTACTG |
| | | CCAGATATTGCAGATATTCCAAGAACTTTCAGGACTCCAGTACCTAGCACTTTAATGCCT |
| | | GGTGGTGTTGACGTAGGCCCTTCTGTGGAATCGTACGAATTGAGAAACACAACCACTTAT |
| | | AAAAGCAGGCCAGATAGACCTAAGCCAGTTTCACCTCCTTGTTATATTATTGACCATATT |
| | | CCGGGTCATTCGCTACTACTTTCTATTAAACTGCCTGGGAAAGTTAATACAAAAGAAGAG |
| | | TTCGCAGCAGCGCCCAATGACACAAGTAGTGGCACCAATGCAATGCTTCCGAGTCCAGAA |
| | | TCGCTGAAAACTAAGCTGAATAGTAACATTCGCGCTGGTGTGACGATACCTTCAATCCCA |
| | | AACCCGATTGCCAATCACACTGTTACTAATTCACCCAATCCCACACTGCAGCCAGTAATC |
| | | CCAGGTGGGGCAGCTAGTAAATCGGTACCTACACCTAGTCTTCCTATAGCACCTCCAGTA |
| | | GCACCACATGATAGCGAAGCGACATTGTTGACTAATAGCAATAATGGTAGCAGTAACAAT |
| | | AACACACAGAATACATAG |
| YCR020W-B | HTL1 | ATGTCACAGAACAACACAATCAGCTCAATGAATCCTGAAAGAGCTTACAACAATGTAACG |
| | | CTGAAAAATTTAACAGCATTTCAGTTATTATCTCAAAGAGAAAACATATGCGAGTTATTG |
| | | AACTTGGTAGAAAGTACGGAAAGACACAATAGTATTATCAATCCTGAAAGGCAAAGGATG |
| | | AGTTTGGAAGAAATGAAAAAAATGCTCGATGCTTTGAAAAATGAAAGGAAAAAGTAG |
| YCR044C* | PER1 | ATGAGGTTAGCTGTGGTTGTGACCCTACTTGTTCACTGTTTCCTAGTGACATGCTCTCCA |
| | | GGAGATAATTTAGATGAGTTTATAGACTGTACGTATGCGTGCGAGTATAACAGAAGATGT |
| | | CCGAATTCCCAAATAAACTACATTGACCCTGAAACCAACATGTTTCATGATATTGAGTTT |
| | | TTCGATACCCCGCCTTTGTACTCTAAGTTATTGTTCTGGGATTGTATCTCAGATTGTGAT |
| | | TACCAATGTCAGCACATCATTACGCGCTGGAGAATTGACGAAGAGGAAGAAATATACCAA |
| | | TTCCATGGGAAATGGCCATTTTTGAGAGTATTGGGGACTCAGGAATTCTTCTCGACGATA |
| | | TTCAGTATAGGTAACTTTATTCCACATTATAAGGGATTTGTAAAGTTTTCTAGAATCATA |
| | | CGCGAAGAGGGAGATAGGAGGAGAAAAAACAGCAGAAGTATACTGATTTGGAACTACCTT |
| | | TACGTTACTGTGGCGGGAATGTTGGCTTGGACAGCAAGCTCGGTCTTTCACTGTCGTGAT |
| | | TTGATCATAACGGAGAAGCTAGATTACTTTTTCGCAGGTTTAACTGTCCTAACAGGGTTT |
| | | CATGCAATATTTGCAAGAATGACTTCCATGTTCCTGTACCCCAAGATAGCACAAGCGTTC |
| | | ACTGCGTCAGTTGCGGCAATCTTTGCCCTGCACATCTTGAGACTCTATGTTGACTGGTCG |
| | | TACACATACAACATGAGATTCAACATTTTTTTCGGTGTTTTACAGTATATTTTATTGATA |
| | | ATGTTATCATGCCAAAACTACCATGCTCTGCAAAAGCAAAAGCTAATGGGCGAATTCAAG |
| | | AAAACCGCGTACTCCAGCTTCAAGCGGCAAATCTTCAAACTGTGTGTCATTCCAATTCTT |
| | | CTTGTGATTGTAACCACAATGGCCATGTCATTGGAACTGTTTGACTTTTTTAGTTACGAA |
| | | TGGCAGATTGATGCCCATGCGCTATGGCATCTGTGCACAATATGGCCCTCGTGGGTATTG |
| | | TACGACTTTTTCCTCGAGGATTATGCCTATTGGGGTAATAGACAATTGTACTAA |
| YCR094W | CDC50 | ATGGTTTCATTGTTCAAAAGAGGTAAGGCTCCACCGCTCACGAAAGAAGGCCCCACTTCT |
| | | AAAAAGCCTCCTAACACAGCGTTTAGACAACAAAGGCTTAAGGCATGGCAACCAATACTG |
| | | TCTCCTCAAAGTGTGCTTCCGTTGTTAATATTCGTTGCATGTATATTTACTCCTATTGGT |
| | | ATTGGACTCATTGTAAGCGCTACTAAGGTACAAGATCTAACAATTGATTATAGTCATTGT |
| | | GATACAAAAGCATCTACAACTGCTTTTGAAGATATACCAAAGAAGTACATTAAATATCAC |
| | | TTTAAAAGTAAAGTTGAAAATAAACCACAATGGAGGCTAACCGAAAATGAAAATGGCGAA |
| | | CAATCATGCGAACTGCAGTTCGAAATCCCAAACGATATCAAGAAATCCATTTTTATATAT |
| | | TATAAAATAACCAATTTTTATCAAAATCATCGCAGATATGTCCAATCGTTTGACACAAAG |
| | | CAAATATTAGGGGAGCCTATCAAAAAAGATGATCTGGATACAAGCTGTAGTCCAATAAGA |
| | | AGTAGGGAAGACAAAATAATATATCCCTGTGGGTTGATCGCTAATTCCATGTTTAATGAT |
| | | ACATTTTCTCAGGTGTTGAGTGGTATAGATGACACAGAAGACTATAATTTAACTAACAAG |
| | | CATATATCATGGAGTATTGATCGTCACAGATTTAAAACCACCAAGTATAATGCTAGCGAT |
| | | ATTGTTCCACCGCCAAACTGGATGAAGAAGTATCCCGATGGGTATACAGATGAAAATCTT |
| | | CCTGATATCCATACTTGGGAAGAGTTCCAGGTATGGATGAGGACTGCAGCCTTTCCCAAG |
| | | TTTTACAAGTTGACGTTGAAAAATGAATCTGCTTCTTTACCGAAGGGTAAATATCAAATG |
| | | AACATTGAGTTGAATTATCCGATTTCACTCTTTGGTGGCACAAAATCATTTGTACTGACT |
| | | ACAAATGGAGCTATTGGTGGTAGAAATATGTCACTAGGCGTACTGTACCTCATCGTTGCA |
| | | GGGCTTTGCGCCTTATTTGGCATCATTTTTTTGGTTAAATTAATCTTCCAACCAAGAGCG |
| | | ATGGGTGATCACACTTATTTGAATTTTGATGATGAAGAAAACGAGGATTATGAGGATGTA |
| | | CACGCAGAGAATACAACATTGAGGGAAATTTTATAG |
| YDL013W | SLX5 | ATGCATAGCGATACTAACGGTCGAACAAAGAGCAATAATAGCCCATCAGACAATAATCCA |
| | | AATGAGACAGTTATATTAATAGATAGTGATAAGGAAGAAGACGCTTCTATTCGCGAGGCT |
| | | AACCTTCCTGTGAGGTTGTACCCAGACAGACGGGTAGGGAGACGACGTGATGCTTTAAAT |
| | | AGGTTTGTGAGGTCAGATTCAAGGAGCAGGAATTCTCAACGGACTCACATAACAGCAAGC |
| | | TCTGAGCGCCCTGATTTTCAAGCTAATAACGACGATATCACTATAATACGAGAAGTTGGA |
| | | CGGTTTTTTGGAGACGATGGACCTATTGATCCTTCTGCACATTACGTCGATCTTGATCAG |
| | | GAGCCAGGTTCTGAAACGTTAGAAACTCCAAGAACAATACAAGTAGATAACACAAACGGG |
| | | TACTTGAATGATAATGGTAATAACAATGAAAGTGATGATGGCCTGACAATAGTTGAAGAG |
| | | AGAACAACACGACCCAGAGTGACACTGAACCTGCCAGGCGGGGAGAGGCTTGAGGTAACT |
| | | GCGACGACAACAGACATACCGATAAGAAGGTCATTTGAATTCCAAGAAGATCTAGGCGCA |
| | | TCACGCAGGCAGTTACTGAGAAGGAGTGCTACAAGAGCCCGTAATTTATTTGTGGACCGG |
| | | TCTGATGAAAACGATGAAGATTGGACAGATGATACCCATAATTTACCAGAAGCTATTCAA |
| | | AGGGCTCGTAGGGAGAGCCGTATGCGAATGAGCAGACGGATTGCAGAAAGACAACGTAGG |
| | | GTACAACAGCAAAGGGTGTCCAGTGACGAAAATATAAGTACATCTATTAGGTTACAATCA |
| | | ATCAGGGAACGAATACAATCATATACGCCTGATATTCGTAGTGCTTTTCATCGTGCAGAG |
| | | TCACTGCATGAATTCAGGTCTATTTTACAAAATGTTGCTCCAATTACTTTGCAAGAATGT |
| | | GAAGAAGAACTAATGGCACTCTTCACTGAGTTCAGAAATCAACTGCTGCAGAATTGGGCC |
| | | ATTGATAGGGTCAGAAATACTCAGGAAGAGGCCTTGAGACTTCACCGAGAAGCATTGGAA |
| | | AGACAAGAAAGAACGGCAGGAAGAGTTTTCCATCGTGGCACATTACGTGAATCAATCACA |
| | | AACTATCTTAATTTCAATGGAGAGGATGGGTTCTTAAGTCGTTTGTGGAGTGGCCCAGCA |
| | | TTGAGCGATGCTGACGAGGAACGACACACTCAAAATATCATTGATATGATCCAAGAGAGA |
| | | GAAGAACGAGAGCGGGATGTGGTCATGAAGAATCTAATGAACAAAACCAGAGCCCAACAG |
| | | GAAGAATTCGAAGCTAGGGCAGCCAGCCTACCCGAAGGTTATAGCGCGTCATTTGATACT |
| | | ACGCCAAAAATGAAATTAGACATTACGAAAAATGGTAAAGAAGAAACCATCATTGTGACA |
| | | GATGACGATTTAGCAAAAACACTCGAGGATATTCCTGTTTGTTGCTTGTGTGGCGCAGAA |
| | | TTGGGGGTCGGAATACCGGATGATTTTACTGGAATCAGTCAAAAGGATCGTGGTGTTTCA |
| | | TTTGAAGGGTTAGTGTCAAAATACAAATTCCACTGCCCCTACCAAACCCTAGCCAGACCA |
| | | TCAATGCTTGATAGAGATTTATCAAAACGAACGTTTATTGCATCGTGTGGTCATGCATTT |
| | | TGTGGTAGGTGTTTCGCCAGGATTGACAATGCAAAAAAAAAATCTAAGATGCCCAAAAAG |
| | | AAACTAGCTCAACTAAAGGGTTCGGCTCATCCGGATAATTATGGCCCCAAATTATGTCCC |
| | | GCAGATTCTTGTAAGAAGCTTATTCGTTCAAGAGGCAGGTTGAAGGAGGTTTACTTCTAG |
| YDL023C* | | ATGATTTCAGTCAATTTTTCACCATTGATCTCTTCTTCGAACACCCACATTTGTACTATT |
| | | GGAGCGAAAACTTCTGGGTATCCCTTACAATTTTCGGCAACCACCTTGGCAATAGTAGTA |
| | | CCCCAGTTACCAGATCCAATCACAGTAACCTTGAAAGGCTTTTCGGCAGCCTTCAAAGAA |
| | | ACAGAAGAGGAACTTCTCTTTCTACCAGCATTCAAGTGGCCGGAAGTTAAGTTTAATCTA |
| | | TCAGCAGCAGCAGACATCTTTATATTATCAATATTTGTGTTTGTGGAGGGGGGGGGTGTA |
| | | CAATATACAATTGTTTCTTGA |
| YDL075W | RPL31A | ATGGCCGGTTTGAAAGACGTTGTCACTCGTGAATACACCATTAACTTGCACAAAAGATTG |
| | | CACGGTGTCTCCTTCAAGAAGAGAGCTCCAAGAGCTGTCAAGGAAATTAAGAAGTTCGCC |
| | | AAGTTACACATGGGTACTGATGATGTCCGTCTAGCTCCAGAATTGAACCAAGCTATCTGG |
| | | AAGAGAGGTGTCAAGGGTGTTGAATACAGATTAAGATTGAGAATTTCCAGAAAGAGAAAC |
| | | GAAGAAGAAGACGCCAAGAACCCATTGTTCTCCTACGTTGAACCTGTCTTAGTTGCTTCT |
| | | GCCAAGGGTCTACAAACTGTTGTTGTCGAAGAAGATGCTTAA |
| YDL106C* | PHO2 | ATGATGGAAGAATTCTCGTACGATCACGATTTTAACACACATTTTGCTACAGATTTGGAT |
| | | TATTTGCAACATGACCAACAACAACAACAACAGCAACAACATGATCAACAACATAATCAA |
| | | CAGCAACAACCACAACCACAACCAATTCAAACTCAAAACCTGGAGCACGACCACGACCAA |
| | | CATACTAATGATATGAGTGCTTCATCGAATGCATCAGATAGTGGACCTCAAAGGCCCAAG |
| | | AGGACTCGCGCAAAGGGTGAAGCACTAGATGTGCTAAAGCGTAAATTTGAAATAAATCCA |
| | | ACACCCTCTTTGGTAGAAAGAAAGAAAATATCAGATCTGATAGGAATGCCTGAAAAAAAC |
| | | GTCAGAATTTGGTTTCAGAACAGAAGAGCTAAATTGAGGAAAAAGCAGCATGGAAGTAAT |
| | | AAGGACACAATCCCCTCGTCACAATCCCGTGATATTGCCAACGATTACGATCGTGGGAGT |
| | | ACAGACAACAATTTGGTCACTACAACAAGTACTTCATCCATATTTCACGATGAAGACCTG |
| | | ACTTTTTTCGACCGTATTCCGCTGAACAGCAACAACAACTATTATTTTTTTGACATTTGC |
| | | TCAATTACTGTGGGAAGTTGGAATAGAATGAAAAGCGGCGCACTGCAAAGAAGGAACTTT |
| | | CAGTCTATAAAGGAGTTGAGAAACCTATCGCCAATAAAGATTAATAACATAATGTCGAAT |
| | | GCCACAGATTTAATGGTTTTGATATCCAAGAAAAACTCAGAAATAAACTATTTTTTTAGT |
| | | GCCATGGCAAATAATACTAAAATTCTCTTCAGGATCTTTTTCCCATTAAGTTCAGTCACG |
| | | AATTGCTCTCTAACTTTAGAAACTGACGACGATATAATAAATAGTAACAACACAAGCGAT |
| | | AAAAACAATAGTAATACTAATAATGATGATGATAACGACGATAACAGTAATGAAGACAAT |
| | | GATAATAGTAGTGAGGATAAGAGGAATGCTAAGGATAACTTTGGAGAATTGAAGCTAACA |
| | | GTCACCAGATCACCCACTTTTGCTGTTTACTTTTTAAATAATGCTCCTGATGAAGATCCA |
| | | AATTTGAACAATCAGTGGTCCATATGTGATGATTTCTCAGAAGGTAGACAGGTAAATGAC |
| | | GCATTTGTTGGTGGTTCGAATATTCCTCACACTTTGAAAGGTTTACAGAAATCATTAAGA |
| | | TTCATGAATTCTCTAATTCTAGACTATAAATCATCGAATGAAATATTACCTACGATCAAT |
| | | ACAGCGATCCCCACTGCTGCAGTTCCACAACAGAATATTGCCCCTCCCTTTCTGAATACA |
| | | AATTCAAGTGCAACAGACTCAAATCCAAATACAAATTTAGAAGATTCTCTCTTCTTCGAT |
| | | CATGATCTGTTATCGAGTTCGATAACCAACACCAACAACGGACAAGGCTCTAATAATGGA |
| | | CGTCAAGCTAGCAAGGATGATACGCTCAATTTACTGGATACTACCGTCAACAGCAATAAC |
| | | AATCATAATGCTAATAATGAGGAGAATCATCTAGCGCAAGAACATTTATCCAACGATGCT |
| | | GATATTGTTGCAAATCCAAATGATCATTTGTTGTCTTTACCGACTGATAGTGAACTCCCA |
| | | AATACTCCAGATTTTTTGAAGAACACTAACGAACTAACTGACGAGCATAGATGGATATGA |
| YDR049W | | ATGAATTCACAAAAGGCGAGTAAAATGACTGGCTCACTAAAAAAGAATGATCTTTATATT |
| | | TTTGATTTGTCTGAGCAACTTCTGAATTCATTGAAATTGATGAGCTTTGACTCCACACTG |
| | | CGAGAAGTTGAAGTAGAGAAGACATCTGATAATGATAGAAACAAGGAGTCTGGAGATCTC |
| | | CAAATAGCAAGAAAGAAGGTCACATCAAATGTTATGCGTTGTAGTGTTTGTCAAATGAGC |
| | | TTTGATTCCAGAAATGAACAAAAGGCCCACTACCAGACTGATTATCATTTGATGAATGTT |
| | | AAGCGTAACCTTAGGGGGCTGGATATCCTATCCGTTGAGGAATTCGATGCGCTGATTTCT |
| | | AAGGAGCATGGTATCAAGTCTGAAGACGAAAATTCTGGCGGCGAGCAAACTTCAAGCGAT |
| | | CACGAAGAAAGTGAGGAAGCCAGCGATCGGGACCCGGACCTACAAACTAATAATTATATG |
| | | GAAACTATAATCGAAAATGATTTGCAGAAATTAGGGTTCCAAAAAGACGAGTCGGATGCT |
| | | ATTAGCCATATTAATACCCAATCGCCCTACATTTATTTCAAATCGAAGTATCTTCAAAAG |
| | | AATGAGGTCTTGGCGATTTATAAGTCTCTATTTAATAAAAGGAGTCTTTCCAACCCCAAT |
| | | GAAGCTTTGACTTTTTGGAATTCGCAAGAGAACCCAATGGCCATTTCAGCTCTGTTTATG |
| | | GTGGGAGGTGGACATTTTGCGGGAGCAATTGTTTCTCATCAAAGACTCAATGTCAAGGGG |
| | | AACGCGCACAAGAAGGACGAAACCTTAATTGAGCAAGCGGTCAATTTCTTAGAGCACAAG |
| | | ACGTTTCACAGGTATACTACCAGAAGGAAGCAAGGTGGTTCTCAGTCAGCAATGGACAAT |
| | | GCAAAGGGAAAAGCTAACTCTGCCGGTTCAGCACTTCGTAGGTACAATGAAAGCGCTTTA |
| | | AAAACAGATATACAGGGTGTGTTGAAGGACTGGGAACCTTATCTGTCAAAGTGTGATAAT |
| | | ATTTTTATTCGAGCTCGCAATGTCAGTGATAAAAAAATTTTTACTGATAATACCGTTCTG |
| | | AACAAGGGTGATGAAAGGATAAAGAGCTTCCCTTTTACCACGAACAGGCCCACTGTATTA |
| | | GAATTGAAGAAGGCATGGTGCGAACTTTCCTACCTGAAAATATTACCTAAGCCTGAACCA |
| | | TTGGCAGTGAAAGAAACAGTACAAAAGTTAGAAGTGTCGAACAAAAAGGACGAATTTAAA |
| | | GAAAAACAAGAACCGTTACTGGAAGAAATCCAAACAGAAGAAATAATTTCTCTGCTGAAG |
| | | AAAGGAAGGGCACCGTTATTGATATCCTTCTTAAAAAAGAATAAGCTTGATGGAAACTTT |
| | | CGGTTAAAGCCTGAGAGTAAATATTCCTTAACCCCTACCATGTTACATTATGCATCGCAG |
| | | CAAGGGATGAAACAGATGGCACTGATTTTGCTCTCAAATATTAAATGCGATCCAACGATC |
| | | AAGAACAGACTAGGAAGGACTGCGTGGGATTTGAATAGAAACGACGACGTGAGACACGCG |
| | | TTTCAAATTGCAAGATACAATCTTGGAGAGTCTTTCACTAATTGGGACGAAACTCATATC |
| | | GGTCAGCCATTGAGCAGAGAACAAGTTGATGAAATCAATGAAAAGAAAAAAGCAATAGAG |
| | | AATGAAAAGGCAGAAAAACTGATTAAATTGGAATTAGAAGCCGCCAAGGAAAAACAAAGA |
| | | TTTGCTAAAGATGCAGAGAGGGGTCCAGGCAAAAAGTTAACCAATATTCCTTCAATTCAG |
| | | CAACAAAATTTGAATTCTTTGACCGATGAACAGAGACGCAGATTGATGAGAGAACAAAGG |
| | | GCTAGAGCCGCAGAAGAAAGGATGAAAAAGAAATACTGA |
| YDR264C* | AKR1 | ATGGTGAACGAATTAGAGAATGTGCCACGGGCGTCGACCTTGACGAATGAAGAGCAAACG |
| | | GTTGATCCTAGCAATAACGACTCACAGGAAGATATTAGTCTGGGTGATTCTAACGAAATT |
| | | ACTTCGTTGGCATCATTAAAGGCTATCCGATCAGGAAATGAAGAGGAAAGCGGGAACGAG |
| | | CAGGTGAATCATAATGATGAGGCCGAAGAAGATCCTCTGTTAACTCGCTACCATACCGCT |
| | | TGCCAAAGAGGTGATCTAGCGACTGTCAAAGAAATGATACATGGAAAGTTGCTCGAAGTT |
| | | AATAACGACGGTGACTCTACGGAACATATCACTGGTTTGCATTGGGCAAGTATTAATAAC |
| | | AGACTTTCTGTAGTGGATTTTCTAGTTTCACAAGGCGCTGATGTCAATGCAAGGGCTGGT |
| | | GCTTTGCATGCCACACCATTACATTGGGCTGCAAGGTATGGGTACGTGTACATTGTCGAT |
| | | TTTTTGCTTAAGCATGGAGCTGATCCCACCATGACCGATGACCAAGGTTTCAATCTACTA |
| | | CACTTATCTGTGAACAGCTCAAATATAATGCTAGTTCTATACGTTTTATTCAACGTTGTC |
| | | AGCAAAGGTCTTTTAGATATAGATTGCCGAGACCCAAAGGGCAGAACTTCACTACTATGG |
| | | GCTGCTTACCAGGGAGATTCCCTAACTGTAGCAGAATTGTTGAAATTTGGTGCCAGCATA |
| | | AAGATCGCAGATACAGAAGGATTCACCCCACTGCACTGGGGCACTGTAAAAGGGCAACCA |
| | | CATGTACTAAAATACTTGATTCAAGATGGTGCAGATTTTTTCCAAAAAACGGATACCGGA |
| | | AAAGATTGCTTTGCTATTGCTCAAGAAATGAACACTGTGTACTCACTTAGAGAGGCTCTA |
| | | ACACATTCTGGATTCGACTACCATGGTTATCCCATTAAAAAATGGTTCAAAAAAAGTCAA |
| | | CATGCTAAGCTCGTTACATTCATAACGCCATTTCTATTTCTAGGAATCGCATTTGCTCTT |
| | | TTCTCTCATATTAACCCTTTGTTTGTAATCATTGTCCTTTTTTTATTGGCCATTGCCACT |
| | | AACAAAGGCTTGAACAAATTCGTTCTGCCGTCATATGGACGAATGGGTGTCCACAACGTT |
| | | ACCTTGTTAAGGTCACCTTTGTTATCCGGTGTTTTTTTTGGCACTTTACTTTGGGTGACT |
| | | ATCGTTTGGTTTTTCAAAGTTATGCCTCGAACATTTTCTGATGAACAATATACTAACATC |
| | | CTAATGCTGGTAATACTTGTTTCTGTATTTTACCTTTTTGGGCAGCTTGTTATTATGGAC |
| | | CCAGGATGTTTGCCAGAAGAAACTGATCATGAGAACGTGCGACAAACAATCTCGAACTTG |
| | | CTCGAAATCGGTAAATTTGATACGAAGAACTTTTGTATCGAAACTTGGATTAGAAAACCT |
| | | CTAAGAAGCAAATTTTCGCCCTTAAACAATGCTGTGGTTGCAAGATTTGATCATTACTGC |
| | | CCTTGGATCTTTAATGATGTCGGACTCAAAAATCACAAAGCATTTATATTTTTTATTACA |
| | | TTAATGGAAAGTGGTATTTTCACATTTCTTGCACTTTGCCTGGAATATTTTGATGAACTT |
| | | GAAGATGCACACGAAGATACATCCCAGAAAAATGGCAAATGTTTCATCCTAGGCGCTTCA |
| | | GATCTCTGTTCGGGATTAATATATGATAGATTTGTCTTTCTGATATTATTATGGGCTTTA |
| | | CTCCAATCTATTTGGGTTGCAAGTTTGATCTTCGTCCAAGCCTTTCAAATATGTAAAGGA |
| | | ATGACCAATACAGAATTCAATGTTTTGATGAAAGAGAGTAAGTCCATTGGCCCCGATGGG |
| | | TTATCTTTCAATGAAAACTTCAATACTACTCCAGAAGGATTTGCGCCGTCGATTGATCCG |
| | | GGTGAGGAGAGTAATGATACTGTACTAGCACCAGTCCCAGGCTCAACGATAAGGAAACCA |
| | | CGCACTTGTTTTGGCGTTTGTTACGCGGTGACTGGTATGGATCAGTGGTTAGCGGTTATA |
| | | AAAGAAACCATTGGCATCAAGGATAGCACCGGACATAATGTGTATTCGATTACATCAAGA |
| | | ATACCAACAAATTACGGTTGGAAAAGAAATGTTAAAGACTTTTGGCTTACAAGCGATATA |
| | | AATGCGCCTTTGTGGAGGCGAATTTTGTATCCTCCTTCGGGCTCTAAAGCTTTGCTCAAC |
| | | GGGATAGAAGTTGACTATTTTAAGCTTTACAAATTGCCAAATAAGGATGTTGAACAAGGA |
| | | AATGATATGGTATGA |
| YDR266C* | | ATGAGCGAATCAGTGAAAGAAAACGTTACTCCGACACGTAACTTCCGTCGTACTCAAGGG |
| | | CCCCAAAATAATACAAAACCTCACAACGACAGGAAGAATTTCAGAAGAAAGCAAAAGAAA |
| | | AATAACCTTTCTGCTGAACCCAACCTCACCACTTCAAGCGCAGATGATACTGATGAAGAA |
| | | AATGAATTATGTGTAATTTGTGCGCGCAAGTTAACATACGTATCTCTAACACCATGCCAT |
| | | CATAAGACATGCCATATCTGTGGTTTCAGACAACGAGCTTTGTACAACAAGAAAAGCTGT |
| | | TTGATTTGCAGAACGGAGAATGAAGAAGTCATGTTTACCGATCGCATAGATGGTGATATT |
| | | TCAGATAAATATAACTTTTGCGAAAAAAATGAAAAATATGGAATAAATTTCACTAGCGAA |
| | | GAGGTTGCTACCGAAACACTAAATCTTTTAAAATTTTTTTGTCCCCTATCGAAGGATGAA |
| | | CAAGTTTGTGACTTTGGTAGTTTCAAGAAATATAATGAACACTTGAAATCTGAACATAAC |
| | | AGAATGATCTGTTTAATATGTGCAACCCATAAGCATGCATTCCCTTGTGAGCTTGAAATA |
| | | TTCACCCAAAATCAATTACGCAACCATCAAACTAAGGGTAATTCAGAAGGATTCAAAGGC |
| | | CACCCTATGTGTGCCTTCTGTTCCGGAAAACGATTTTATTCAGACGACGAATTGTACATT |
| | | CACATGAGAAATCAGCATGAAAAGTGCCATATTTGTGATAAGATGAACCCCGCTTCTCCT |
| | | CAATACTTTAAGGATTATAATCAGCTTTTTGACCACTTTAAACATTCGCATTACGTTTGC |
| | | ACCGTCCAAACTTGCTTAGATAACAAGTTTGTCGTATTTAAAGACGAGTTGGAGTTACAA |
| | | GCCCACATTTTGCAGGAGCATGGTAATATCCTGAAGGGAAAGCCAAAGTTTTTCCAGTCA |
| | | GAACTTTCTACTTTTATCTCGGCTCCATCTAGAGTGATCAGGGAAAGAGATGATTACGAC |
| | | CTTCCATCTATATCTTCCCTACCGGGTTCATCATCCGGCTCTAGAACTGATGTGAGATCT |
| | | GCTAGTTCTCCCGAGGAAAGTAGACTAAGATTGGCAGAAAGAGCAAAATACTATCTGGAA |
| | | AATTCCAAAGAAGACTTCAATAAGTTTTCTTCTTATAACGAAGATTATTCTAAAGGTCGC |
| | | TTAAGTGCTGAAAAATTATTGGAATCATATAAATTGTTATTCACAAAACCAAATGCGGAT |
| | | GTATATTTATTGATTCATAACTTAGCTGAAACCTTTCCCAAAAACTCCTCCAAATACAAT |
| | | AATCTGAACGCCATATATGAACAAAGAGAGCAAACCCTTGCACGACAAACTTCACTACCT |
| | | TCTTTATCTAGTGATTCTTCCCTTTCAATGTCAATAGGCAGGGGCCATTGGGGTGGAACA |
| | | AACGATGGGGGCAGCGCTGGTGCAGCATTAGGCGTTAGGAATATCAAAAATCTTCCAACC |
| | | CTGAAAAGTCCTTCAGCGTCGTATGATCCCTTTGCAACCACCGTAAAAAAAAACACTCTG |
| | | AGACCCGTGCAAAACATTAAAAGAACTACACCTCAATCAGTTAGTTATCGAACTTCCACT |
| | | AATACAGTCGCCTTCTCTCCAACCTATTTGGAAAGTAAAAAGGGTTCCTCTTCTACTTCT |
| | | CTAAATAATAGCAAAGATAAATTGAAAAGTCTCAACCTACCTCAACTACCTCCACCAAAG |
| | | CCAAAGGTGCAAATTCCAGGTCTGAACAGACCCCAAATTGCTGATCCTAAGCAGTGGGGT |
| | | AAAAAGTCAAGCACACAAGATACTAACGTGCACGATAATTTGAGGGAATTGAACACAACT |
| | | AGTGGAGGAAATAAAAAGAAAGGCAAACAAAAGCAGCTGTTATTCCACATTGGTGTATAG |
| YDR433W | | ATGACAATCCTCCACCAATCAGAAGATCAAATAGAGGTGGCTTCAGAGGTCGCGGCGGCT |
| | | TCAGAGGCGGCTTCAGAGGTGGCTTCAGAGGCGGTTTCTCCAGAGGCGGCTTCGGTGGCC |
| | | CCAGAGGTGGATTTGGTGGTCCAAGAGGTGGTTACGGTGGCTATTCCAGAGGTGGCTACG |
| | | GTGGCTACTCCAGAGGCGGATATGGTGGCTCCAGAGGTGGTTACGATAGTCCTAGAGGTG |
| | | GTTACGATAGTCCAAGAGGTGGTTATTCCAGAGGTGGCTATGGTGGTCCAAGAAATGATT |
| | | ACGGTCCTCCAAGAGGTAGCTACGGTGGTTCAAGAGGTGGTTATGATGGTCCAAGAGGCG |
| | | ATTATGGTCCTCCAAGAGATGCATACAGAACCAGAGATGCTCCACGTGAAAGATCACCAA |
| | | CCAGGTAAGCCATTTATATAG |
| YDR442W | | ATGAGCAGGAAAACGTTGCCGGAAAAAGTTTATTTATCCGAGAGAATAATTGATGAAGAA |
| | | GTAGCGGTATGCACAGTAGCGGCGGAAGTATTAGCAATTTTTACTCTGGTGTGCACAAGA |
| | | GTGTTCATCATTTTTTTCACGGCTAGGATATGCCATGGAATATGGCCATCTTCGCCATCA |
| | | GAGAGACCGTACCACACGTTTAGAGCAGCCAGGTTGCGAAACTCTTCTAAGATGGTTTCC |
| | | AGCAATTGTGTACTATCAGAATGTGGACAGTTTAAAAGGTTGACTGCGAATTTGTCCCAA |
| | | ACCGTATCACCGTCGCATTTTTTGAATTTGATCAAAGCACCACTCCTAATAGCACAGCGA |
| | | TGCTGTGAGTGTGCCAGTGGGAACGGGTGCTGA |
| YDR495C* | VPS3 | ATGGTAAAAAAGAAAACGAATAATGACAAAGGAAAAGAAGTAAAGGAAAATGAAGGAAAG |
| | | CTCGATATAGATAGCGAATCCTCACCTCACGAAAGAGAAAATGACAAAAAAAAAACAGAA |
| | | GATGATAGTCTACGTGCAACAGAAAGTGAAGAGACGAATACACATAATGCTAACCCAAAT |
| | | GAAACAGTACGAGCTGATAAATTTTCCCAGGAAGAGAGTCGACCAATCGAAGACAGTCCA |
| | | CATACTGATAAAAACACAGCACAAGAAAGCTGTCAGCCTAGCAGCGCAGAAGATAATGTC |
| | | ATAAATACAGATATTACAAGCTTAAATGAAAAAACCTCCACGAATGATGAACAGGAAAAA |
| | | GGGTTGCCACTGAAAATAAGCGAAGGTCCTTTTACAATCTCTACCCTATTAGACAATGTA |
| | | CCATCTGACTTGATATACACATGTTGTGAGGCGTATGAGAACCACATATTCTTGGGAACA |
| | | ACTACAGGTGATCTTTTACATTACTTTGAACTTGAACGTGGAAACTATATGCTTGTGTCG |
| | | CAAACAAAGTTTGATGCAGAATCAAATTCGAAGATAGACAAAATCCTTCTTTTACCTAAA |
| | | GTAGAAGGCGCACTAATATTATGTGATAACGAACTAGTTTTATTTATTCTCCCAGAATTT |
| | | GCACCAAGGCCCAACACAACTAGACTTAAAGGTATTAGTGATGTCGTTATTTGTAATTTT |
| | | TCCCGAAGCTCGAAGGCTTATAGAATATATGCATTTCACGCAGAAGGCGTAAGACTACTC |
| | | AAAATATCTGCAGATTCATTAGTACTTACCAAAGCTTTTAATTTCAAACTAATAGATAAA |
| | | GCTTGTGCGCACGAAGAAACATTAATGGTTTCGAAGTTAAATAGTTACGAACTTATAAAT |
| | | CTAAAGTCGTCACAGGTAATTCCATTATTTCGAATAAGTGAGACTGACGAAGACTTAGAA |
| | | CCTATAATAACCAGTTTCAATGAACAGAGTGAATTTTTAGTTTGTTCTGGAGGAGGATCA |
| | | TACGATAGTGGGGCGATGGCCTTAGTGGTAAATCATCATGGCGACATAATAAAAGGAACT |
| | | ATAGTGCTAAAAAATTATCCAAGAAATGTAATTGTTGAGTTTCCCTACATAATCGCTGAG |
| | | TCTGCTTTCCAGTCGGTTGACATATATTCTGCCTTGCCGAGTGAAAAATCCCAGTTGTTG |
| | | CAAAGTATAACTACTTCTGGATCGGATTTAAAAATTTCAAAATCTGACAATGTATTCACT |
| | | AATACCAATAATTCTGAAGAATTCAAAGAAAAAATATTCAATAAGTTGAGACTTGAGCCT |
| | | CTGACACATAGTGATAATAAATTTAGAATTGAAAGAGAACGCGCCTTTGTTGAAGAGTCT |
| | | TATGAAGAAAAGACCTCTTTAATCGTTTATAATAATTTAGGGATTCATTTATTAGTTCCA |
| | | ACGCCGATGGTCTTACGTTTTACATCATGTGAAGAGTCCGAAATTGATAATATTGAAGAT |
| | | CAATTGAAAAAGCTTGCGAAGAAGGATTTAACAAAATTTGAGCACATTGAAGCGAAATAT |
| | | TTGATGTCTCTTTTATTGTTTCTAATGACCTTACATTACGATCACATAGAAGATGAGGTA |
| | | ATGAAAAAGTGGTGTGACTTTTCTGACAAAGTTGACATCAGAATATTATTTTATATGTTT |
| | | GGCTGGAAAGTCTATAGCGAGATCTGGTGCTTCCATGGGTTAATAAATATAGTTGAAAGG |
| | | TTGAAAAGCTTGAAGCTAACTAACAAATGCGAGAATATTCTAAAAATGCTTCTGATGATG |
| | | AAGAATGAACTAAAGAAAAAGAATAAAACTGGACTCTTAACGAATGACTTTGATGATATA |
| | | ATGAAAACTATTGATATAACTTTATTCAAATTAAGATTGGAAAAAAAGGAAACTATAACT |
| | | GTTGATATGTTTGAACGCGAAAGTTACGACGAAATTATCAGAGAGATTAATTTACATGAT |
| | | GATAAACTTCCTAGGATAGAGTTACTAATTGAGATATATAAAGAAAAAGGGGAATATCTT |
| | | AAAGCACTAAATCTTTTGAGGGAAGCTGGGGATTACATTTCACTAGTTTCATTCATTGAA |
| | | GAAAATCTCAAAAAACTTCCTGAGGATTATATCAAAGAAAGAATAGCCGATGATCTTCTC |
| | | CTCACTCTTAAACAAGGCGACGAAAATACTGAGGAATGCGCCATCAAAAAGGTTCTTAAA |
| | | ATATTGGACATGGCATGTATTAATAAAAACGACTTTTTAAACAAAATACCCGCAGAAGAA |
| | | ACATCCCTGAAGGTATCATTCATAGAACAACTCGGTGTCCAAAACAGCAACGATTCCAAA |
| | | TTTCTCTTTAACTATTATTTGGCGAAATTGCGAGAAATAATCAACCAAAGTAACATATGG |
| | | TCCATTCTTGGAGATTTTATCAAAGAGTACAAAGACGATTTCGCGTATGATAAGACAGAT |
| | | ATAACAAATTTTATTCATATCAAATTAAAGCATAGTCTTCAGTGCGAAAATTTTTCCAAA |
| | | TATTATGAAAAATGTGAAAACTTGAAATCGGAAAATGAAAAAGATGATGAGTTTATTAAT |
| | | TTCACCTTCGACGAAATTTCCAAGATCGATAAAGAACACATATTGACCTTGTTATTTTTC |
| | | CCCAATGAATTAACAAACTGGGTTTCTTCCGAAGAATTGCTGAAAATATATTTGTCATTT |
| | | AATGATTTCAGAAGTGTAGAAAAATACATAGGCAAGCAGAACTTGGTTGCGGTGATGAAA |
| | | CAATACTTGGACATATCTTCTTTGAATTACTCAGTTGAATTAGTGACTAATTTGCTACAG |
| | | AGAAATTTTGAACTTTTAGATGATACAGATATTCAATTAAAGATCCTCGAAACTATACCT |
| | | TCCGTATTTCCCGTACAAACAATATCCGAATTGCTTTTAAAGGTACTTATTAAGTATCAA |
| | | GAAAAGAAGGAGGAATCTAATTTGCGGAAATGTTTGTTGAAAAATCAAATATCCATTTCT |
| | | GACGAACTAAGCCGGAATTTTGATAGTCAAGGATAA |
| YDR532C* | | ATGGATACAGGGAGTGCGAGCATAAAGGACTATGAGACCGTGTTAACGGATATTGAGGAT |
| | | TCTATTGCTGTGAGCAGCGAAGAAGTGCTAAATAACCAAGAACTAAGGCTCAAAAATACA |
| | | TTGCACGAAATTACTTCCAGCATTCTGGCAATAAATGAAGAGAACAAGTTTGTGAATCCT |
| | | CTTAGGAACGATGAGAGCTTAGACGTAGAGGGAAAGGAGGTCTTTGTAAATCCAAAAATT |
| | | CTGAGTGCAAAAATCAAGGAGTTCAATAAACTAATGGAGTTACTTAAGTTGACTTACTTG |
| | | GAACAGGAGACATTGGACTATTTCTTCAGGTTTACTTTGTCCTCGACGAAACCTTTGCAG |
| | | TTAGATTCCGAAAAAGACCCCCAATTTGTAAAACTTAATGAAAGAGTTAATGATTTAAAG |
| | | GAAGAAATTTCGAATGTTCAAGAAAGCAAAATAGAACAAATCAAAGCTGAAATCCAAGAG |
| | | ACCGGTCACAACTTTGCAGAAAGGCAAGATTTAATAAATGAGTTGTATCTGGAAGCCACT |
| | | GGTGATATTGAAAATTGTTGGGATTCGCTCAATGAACTAAAAAATTTAACAAACAAAGAA |
| | | GATAAGAATATGATGGGAGAAAAAGATACGATATTAAATTCAAGTGATTCAGATGATTTT |
| | | GTGGAAGAGACATACACGAATTGGCAAAAACTACTATTTTTGCAGAAGCAAAATCAAAGG |
| | | TTAACGAAAGAGCTCAAGGAAATGCATGAAGTTAAAAATCAAATAATACGAAAAGGAGAG |
| | | CAATCAAAAAAAGAGGATTCTGGACATTTAATGGCCAATGAATCAGAATTATGTCAGTCA |
| | | ATTAATTTGCTAACAAAATTTTGGGAAAAGCACTTTTTACTCAAAGGGTCAAAGACGACT |
| | | ATATTAAATTTTGAAATCTTTACCCAGCTTGGAAAAGTACAATTTGAAATAAAGGATATG |
| | | CAATATATAATTGCCATATCTTTAAGCGATTTAAAAAGGCCAATGATAAAAGACATTACT |
| | | ATTTTACAAAAAGCGGGTGGCAATATAGTCACAGACATTGAAGCGAGCAGTAAATTTAAC |
| | | AATAAGTACAGAAATAATACTAAGGTGCAAATCTTTGAGGTAATGGATGACATTATAAGC |
| | | GAGCTAACAAACGAATAA |
| YEL044W | IES6 | ATGAGCGGTAGTAGGGGCAATAGCAGCAATAGTAGCGTCAGTAATAATAGTAATAATAAC |
| | | AATAATAATGACGGGGGCGACGAGAGATTGCTGTTTCTAAGAAGCGTGGGCGAACGCAAT |
| | | GAAATTGGCTTTCCCTCTAGATTCAAGTCGGCGCATTACAAGAAACCGACAAGAAGACAC |
| | | AAATCAGCGAGGCAGTTGATCTCGGACGAAAACAAGCGGATCAACGCCTTGTTGACCAAG |
| | | GCTAACAAAGCTGCAGAGAGTTCTACTGCTGCTAGGCGACTTGTGCCCAAAGCGACGTAC |
| | | TTTAGCGTGGAAGCGCCACCGTCTATCAGGCCTGCCAAGAAGTACTGCGATGTTACTGGG |
| | | TTGAAGGGCTTCTACAAGTCGCCTACGAACAACATTCGGTATCACAACGCAGAAATCTAT |
| | | CAGTTAATCGTAAAGCCGATGGCCCCCGGCGTCGATCAGGAGTACTTAAAATTGAGAGGG |
| | | GCCAACTTCGTTCTAAAATAG |
| YER070W | RNR1 | ATGTACGTTTATAAAAGAGACGGTCGTAAAGAACCTGTCCAATTCGATAAGATTACCGCT |
| | | CGTATATCACGCTTATGCTATGGTTTAGATCCAAAACATATCGACGCCGTTAAGGTCACC |
| | | CAACGTATCATTTCTGGTGTCTATGAAGGTGTCACAACAATCGAACTAGACAACTTAGCC |
| | | GCTGAAACATGCGCTTATATGACTACTGTTCATCCAGATTACGCCACCCTAGCGGCCAGA |
| | | ATTGCCATTTCTAATTTACATAAACAAACCACAAAACAATTTTCTAAGGTTGTCGAAGAT |
| | | CTTTATAGATACGTCAATGCTGCTACTGGTAAGCCCGCTCCCATGATCTCTGATGATGTC |
| | | TACAATATTGTCATGGAAAACAAGGATAAATTGAACTCCGCAATTGTCTATGACAGAGAT |
| | | TTTCAGTACAGTTATTTTGGTTTTAAAACTTTGGAACGTTCTTATTTACTAAGAATCAAC |
| | | GGTCAAGTGGCCGAACGTCCACAACATTTAATTATGAGAGTCGCACTAGGCATCCACGGT |
| | | AGAGATATCGAGGCTGCTTTAGAAACGTATAACTTGATGTCTCTAAAATATTTTACTCAC |
| | | GCCTCTCCAACGTTGTTCAATGCCGGTACTCCAAAACCTCAAATGTCCTCTTGTTTCTTG |
| | | GTTGCCATGAAGGAGGACTCTATCGAGGGGATTTACGACACCTTGAAGGAATGTGCTTTG |
| | | ATTTCCAAAACTGCTGGTGGTATTGGTCTACATATCCATAACATTCGTTCAACTGGTTCT |
| | | TACATTGCTGGTACAAACGGTACTTCTAACGGTTTAATTCCTATGATTCGTGTTTTCAAT |
| | | AACACTGCCCGTTATGTTGACCAGGGTGGTAATAAAAGACCTGGTGCGTTTGCCCTTTAC |
| | | CTGGAACCATGGCATGCTGATATATTTGATTTTATTGATATTAGGAAGAACCACGGTAAA |
| | | GAGGAAATTCGTGCAAGAGATTTGTTCCCTGCTCTATGGATTCCTGATCTTTTCATGAAG |
| | | CGTGTCGAAGAAAATGGGACCTGGACATTATTCTCTCCAACATCAGCTCCTGGTTTAAGC |
| | | GATTGTTACGGTGACGAGTTTGAGGCTCTATATACCCGCTACGAGAAAGAAGGTCGTGGT |
| | | AAGACTATCAAAGCCCAAAAATTATGGTATTCCATTTTGGAAGCTCAAACTGAAACTGGT |
| | | ACACCTTTCGTTGTTTACAAGGATGCTTGTAACAGAAAATCTAATCAAAAAAACTTAGGT |
| | | GTCATCAAGTCATCAAACTTATGCTGTGAAATTGTTGAATACTCAGCTCCAGATGAAACT |
| | | GCTGTTTGTAACTTGGCTTCCGTTGCCTTACCAGCATTCATTGAAACTTCTGAGGATGGT |
| | | AAGACTTCCACATACAACTTCAAAAAATTACATGAAATTGCTAAAGTTGTTACTCGTAAT |
| | | TTAAACAGAGTCATTGATCGTAATTACTACCCTGTTGAAGAAGCGAGAAAATCCAATATG |
| | | AGACATAGACCAATTGCTTTGGGTGTTCAAGGTCTCGCTGACACTTTCATGCTGTTACGT |
| | | TTGCCATTTGATTCTGAGGAAGCCCGTTTGCTAAATATCCAAATCTTTGAAACTATTTAT |
| | | CATGCCTCCATGGAAGCTTCTTGTGAACTAGCTCAGAAGGACGGTCCATACGAAACTTTC |
| | | CAAGGATCTCCTGCTTCTCAAGGTATACTACAGTTTGATATGTGGGACCAAAAACCCTAC |
| | | GGCATGTGGGATTGGGACACCTTAAGAAAAGATATCATGAAGCATGGTGTTAGAAATTCC |
| | | TTGACCATGGCACCAATGCCTACTGCATCCACATCCCAAATATTGGGTTATAATGAATGT |
| | | TTCGAACCAGTCACTTCCAATATGTACTCCCGTCGTGTCTTATCCGGTGAATTCCAGGTT |
| | | GTGAACCCTTACTTACTGCGTGACTTGGTTGATTTAGGTATTTGGGATGAGGGTATGAAA |
| | | CAGTATCTGATTACACAAAATGGCTCCATTCAAGGCTTACCAAACGTTCCACAAGAATTG |
| | | AAGGACTTATACAAGACTGTTTGGGAAATTTCACAAAAGACTATCATTAACATGGCAGCC |
| | | GATCGTTCTGTCTATATTGATCAATCTCATTCTTTGAATTTGTTCTTACGTGCCCCAACT |
| | | ATGGGTAAACTAACAAGTATGCATTTTTACGGATGGAAGAAGGGATTGAAGACCGGTATG |
| | | TACTATTTGAGAACCCAAGCTGCATCTGCTGCAATTCAATTTACTATTGATCAGAAGATT |
| | | GCGGATCAAGCTACAGAAAACGTTGCTGATATTTCCAACTTGAAGCGTCCATCATATATG |
| | | CCTTCCAGTGCAAGCTACGCTGCCAGCGATTTCGTGCCCGCAGCTGTGACTGCAAACGCA |
| | | ACTATTCCATCTCTAGATAGCTCCTCGGAAGCTTCAAGAGAGGCATCTCCAGCTCCAACA |
| | | GGTAGCCACTCATTAACTAAAGGAATGGCAGAATTAAACGTTCAAGAGTCTAAGGTAGAA |
| | | GTTCCTGAAGTACCTGCCCCAACTAAGAATGAAGAAAAAGCTGCCCCCATCGTTGATGAT |
| | | GAGGAAACCGAGTTCGACATTTACAACTCTAAGGTTATAGCATGTGCTATTGATAACCCA |
| | | GAAGCTTGTGAAATGTGTTCGGGTTAA |
| YER087W | AIM10 | ATGTTGAAATATCGTACATTGTCTCGATCATGTCATATTTTTCACCCAAAATCTCTTTCA |
| | | AACAATACCCTGAAATCTGAAACTACTCAGGAACTACTTCAAACTGTGGGATTCGTAAGA |
| | | CGCTCGCAGGTTGGGTTGTTCCAATGGCTGCCACTTGGACTCAGATCACTTAATAAAGTA |
| | | TCAAATGCTATACGGAACCGAATGGATAGCGATGGTGGAGCCATAGAAGTTTCATTAAGT |
| | | GCTATCTCGTCAAAAGCATTATGGCAAGCGACGGATCGCTGGAACAACAGTGAATTATTT |
| | | AAATTGAAGGATTCTAAAGGGAAGCAGTACTGCTTGACAGCAACATGTGAGGAGGACATT |
| | | ACAGATTTAATGAAAAATTACATTGCTAGTTATAAAGATATGCCTATCACAATATACCAA |
| | | ATGACAAGAAAATATAGGGACGAAATCAGACCCAGGGGTGGTATCTTACGTGGACGAGAA |
| | | TTTTTAATGAAGGATGCGTATTCGTTTGCTAGTAATGAAGAGGATGCGTTTGCCAGTTTT |
| | | CAAAAGCTAGATGATACTTACAATAAGATATTCAAAGATCTGAAAATCCCCTTTGTTAGT |
| | | GCGTGGGCAGATAGCGGTGACATTGGCGGTGAGTTTAGCAAAGAATTTCACCTGATTCAT |
| | | GAATCCGGTGAGGATACCTTGATGAGTTGCAAACATTGCGGCGATATATCAACTTTGGAT |
| | | ATGTCCCAGTCATATCCTGAAAAAGATGGTCAATATTCAGGTGATGTCGATTGTAAGTAT |
| | | GCTTTGACTAAGGATCACTCAACATTGATATGTTTCTACTATCCAAAGGATAGACAATTG |
| | | AATTGGAATTTAGCATTAAATGCTATGGATAAAGACATCGATTTGACGTTGAGGAACAAA |
| | | CCAAATGATCACGTCTTACAAGTCTACGAAAAAGATAACGAAGATATTATGTTCAGTAAA |
| | | ATATTAAGAGTTATGGACTGTAGATTGAATTCAAAATCCAACTTTCCTGATTTTCCACTA |
| | | AAGAAATACCTGAAAAATAATTTTGGTCAAATCAGTGACGTTTCTATAGTGGATGCACAG |
| | | GAAAACGAAATCTGTGGCAAATGTGAGGAAGGAAGGTTAGAACCTCTCAAAAGTATTGAG |
| | | GTAGGCCACATATTTTTGCTGGGTAATAAATACTCGAAGCCGTTAAATGTCAAGTTTGTC |
| | | GACAAAGAAAACAAAAACGAGACATTTGTCCATATGGGTTGCTACGGCATTGGAGTAAGC |
| | | AGGCTAGTTGGAGCAATTGCTGAACTAGGGCGTGATTCCAATGGATTTAGATGGCCCGCT |
| | | ATAATGGCTCCATACAAAGTATCGATTTGTACTGGACCAAATAATCCTGAAAACTCACAG |
| | | AGGTTACAGGATGTTAAATCAGAGCTGCTGAATGATCCTACCATGCAAAATTTACAGAAT |
| | | GACATCCTTGACCAGTTTAATGAAAAACTGGGTATAGGTGCTAGAATAAAATTATCGCAT |
| | | GCCATGGGTATACCATTATGTGTTATCGTGGGCTCAAAAAGTTGGCCCAATGTGGAAATT |
| | | GAAGTTCGTGGCATAAGATGGGGAGAAAAGGACCTGTGGAGAAAGCAGTTTGAAAAAAGA |
| | | TGTAGCGAACTGCAATGGAAGTGTACCAAAAATGAACATGGAATTGAGAAACATACTGTT |
| | | CCAATCCAGCATTTAGCGGAAGTCATTGGCGTATTGCTAAAGGATATGTAG |
| YER151C* | UBP3 | ATGAACATGCAAGACGCTAACAAAGAAGAGTCGTACTCGATGTACCCGAAAACCTCTTCT |
| | | CCACCACCACCTACGCCAACCAATATGCAGATTCCTATTTATCAAGCGCCTTTGCAGATG |
| | | TACGGCTACACTCAGGCCCCATATCTATACCCCACACAAATACCTGCCTATTCGTTTAAT |
| | | ATGGTCAACCAAAACCAGCCAATCTACCATCAAAGTGGCAGCCCACATCACTTGCCTCCG |
| | | CAAAACAATATTAACGGCGGAAGCACTACCAATAACAACAACATTAACAAGAAGAAGTGG |
| | | CACTCTAATGGCATTACCAATAACAATGGAAGCAGCGGTAATCAAGGCGCCAACTCTAGC |
| | | GGTAGCGGCATGAGCTACAACAAATCCCACACCTACCATCACAATTACTCTAACAATCAT |
| | | ATCCCCATGATGGCCTCTCCAAACAGTGGCAGCAATGCGGGCATGAAAAAACAGACCAAC |
| | | TCTTCCAACGGCAACGGTTCTTCGGCTACTTCACCATCGTACTCTTCCTACAACTCTTCT |
| | | TCACAGTATGATTTATACAAGTTTGATGTCACTAAATTAAAGAATCTCAAGGAAAATTCA |
| | | TCAAACTTGATTCAATTGCCACTGTTCATAAACACTACGGAAGCAGAATTTGCTGCGGCA |
| | | AGTGTCCAAAGGTACGAATTAAACATGAAGGCTTTGAACCTAAACTCTGAAAGCTTAGAG |
| | | AACTCATCTGTAGAAAAGAGCTCTGCCCATCATCACACAAAAAGCCATAGTATACCAAAG |
| | | CATAATGAGGAAGTAAAGACAGAAACACATGGGGAAGAAGAAGATGCTCATGATAAAAAA |
| | | CCACATGCGAGCAAAGATGCGCACGAGCTTAAAAAGAAAACTGAAGTAAAGAAAGAGGAT |
| | | GCTAAGCAAGACCGTAACGAAAAAGTTATACAGGAACCTCAAGCTACTGTTTTACCTGTA |
| | | GTGGATAAGAAGGAACCAGAGGAATCTGTTGAAGAAAATACTTCCAAGACATCTTCACCT |
| | | TCACCATCTCCTCCAGCAGCAAAATCCTGGTCCGCCATAGCATCAGATGCGATTAAAAGT |
| | | AGACAAGCTAGTAACAAAACAGTCTCCGGATCGATGGTCACTAAAACACCAATTTCTGGT |
| | | ACGACCGCAGGCGTTTCATCAACAAACATGGCTGCGGCGACTATAGGTAAATCCAGCTCT |
| | | CCCCTGTTGTCCAAGCAGCCTCAGAAAAAGGATAAAAAATACGTTCCACCTTCTACAAAG |
| | | GGTATTGAGCCACTGGGTTCGATTGCGTTAAGAATGTGTTTTGATCCCGATTTCATTAGT |
| | | TACGTTTTACGGAATAAAGATGTTGAAAACAAAATACCAGTCCATTCCATTATTCCAAGA |
| | | GGCATAATTAACAGAGCCAACATTTGTTTTATGAGTTCTGTGTTACAAGTGTTACTCTAC |
| | | TGTAAGCCATTTATTGATGTAATTAACGTTCTCAGTACACGGAATACCAATTCAAGAGTC |
| | | GGCACATCATCCTGTAAATTATTAGATGCTTGTTTGACTATGTATAAGCAATTCGATAAG |
| | | GAAACCTATGAGAAAAAATTCCTAGAGAATGCTGATGATGCTGAAAAAACCACGGAAAGT |
| | | GATGCAAAAAAATCATCAAAATCCAAGAGTTTCCAACACTGCGCCACTGCCGATGCTGTC |
| | | AAACCTGACGAATTTTACAAAACTTTGTCTACTATACCGAAGTTCAAAGACTTGCAATGG |
| | | GGCCATCAGGAAGACGCAGAAGAATTTTTGACCCACTTATTGGACCAATTACACGAGGAA |
| | | TTAATTTCTGCAATTGATGGCTTAACCGATAATGAAATTCAAAATATGCTGCAAAGTATT |
| | | AATGATGAACAATTGAAAGTTTTCTTTATTAGAAATTTGTCACGTTATGGAAAAGCAGAG |
| | | TTTATCAAAAATGCTAGTCCTAGACTGAAGGAGTTGATAGAAAAATATGGCGTGATCAAT |
| | | GATGACTCTACCGAAGAAAATGGTTGGCATGAAGTGAGCGGATCTAGCAAAAGAGGCAAG |
| | | AAAACTAAGACCGCTGCCAAGAGGACTGTCGAGATTGTTCCATCACCAATCTCCAAACTT |
| | | TTCGGTGGCCAGTTCAGATCTGTGTTAGATATACCGAACAATAAGGAATCTCAATCGATT |
| | | ACACTCGATCCGTTCCAAACAATTCAATTGGACATTTCAGATGCTGGTGTGAATGATCTA |
| | | GAAACTGCATTCAAAAAATTTAGTGAATACGAATTGCTACCCTTTAAGTCCTCGTCAGGG |
| | | AATGATGTCGAGGCCAAGAAGCAGACTTTTATTGATAAATTGCCGCAAGTTCTTTTAATC |
| | | CAATTCAAAAGATTCTCATTCATAAATAATGTGAACAAAGACAACGCAATGACGAACTAT |
| | | AACGCGTACAATGGACGTATTGAGAAGATCAGGAAAAAAATTAAATATGGTCACGAGTTA |
| | | ATCATACCTGAAGAATCAATGTCTTCCATAACATTGAAAAACAACACCTCAGGGATTGAT |
| | | GATAGAAGATATAAGCTAACCGGAGTTATATACCATCATGGGGTAAGTTCCGATGGCGGT |
| | | CATTACACAGCGGATGTTTATCATAGCGAGCACAACAAATGGTATAGAATAGATGATGTA |
| | | AATATTACCGAACTAGAGGACGATGACGTTTTGAAAGGTGGCGAAGAAGCTTCTGATTCG |
| | | AGGACTGCCTATATTTTAATGTATCAAAAGAGAAATTAA |
| YFR010W | UBP6 | ATGAGCGGAGAAACGTTTGAGTTCAATATTAGACATTCTGGTAAAGTTTACCCAATAACA |
| | | CTTTCCACTGATGCTACTTCAGCAGATTTGAAAAGCAAAGCAGAGGAATTGACCCAAGTC |
| | | CCAAGTGCCCGCCAAAAATACATGGTTAAAGGTGGCTTGTCTGGCGAAGAGTCCATTAAA |
| | | ATATATCCCTTAATCAAGCCAGGATCGACAGTAATGCTATTGGGGACTCCAGATGCTAAC |
| | | CTGATTTCTAAACCAGCCAAAAAGAATAATTTCATTGAAGACCTTGCGCCTGAGCAACAA |
| | | GTCCAACAATTTGCTCAATTGCCTGTTGGTTTCAAGAATATGGGCAACACCTGTTATCTG |
| | | AATGCTACCCTACAGGCTTTATACAGAGTGAACGATTTAAGGGATATGATTCTTAATTAT |
| | | AACCCTTCTCAAGGTGTGTCTAACAGTGGTGCACAAGATGAAGAGATTCACAAACAAATC |
| | | GTTATTGAAATGAAGCGTTGTTTTGAAAATTTACAGAATAAAAGTTTCAAGAGTGTTTTG |
| | | CCAATTGTGTTATTAAACACGCTAAGAAAGTGTTATCCACAATTTGCTGAACGTGATTCA |
| | | CAAGGTGGGTTCTATAAACAGCAAGACGCTGAGGAGTTGTTTACACAACTATTCCATAGT |
| | | ATGAGTATTGTTTTTGGTGACAAATTTTCCGAAGATTTCAGGATTCAATTTAAAACTACC |
| | | ATCAAAGACACAGCTAATGATAACGATATTACTGTTAAAGAAAATGAAAGCGATTCTAAA |
| | | TTACAATGTCATATTTCTGGTACTACAAATTTCATGAGAAATGGGCTCCTGGAAGGTTTG |
| | | AATGAGAAAATTGAAAAAAGATCAGACTTGACTGGCGCCAATTCCATCTATAGCGTCGAA |
| | | AAGAAAATATCAAGATTACCAAAGTTTTTAACTGTTCAGTACGTTAGATTTTTCTGGAAA |
| | | AGGTCAACCAACAAAAAATCTAAAATATTGCGTAAGGTCGTTTTCCCATTTCAATTAGAT |
| | | GTTGCAGACATGCTTACCCCAGAATACGCAGCAGAGAAGGTAAAAGTTCGTGACGAACTG |
| | | AGAAAAGTTGAAAAGGAGAAAAATGAAAAGGAAAGAGAGATCAAAAGGCGTAAATTTGAC |
| | | CCATCATCCAGTGAAAATGTCATGACACCAAGAGAACAATATGAGACACAAGTGGCTCTT |
| | | AACGAAAGTGAAAAAGATCAATGGCTCGAAGAGTATAAGAAACATTTTCCTCCAAACTTG |
| | | GAAAAAGGTGAAAACCCATCTTGTGTTTATAACTTGATCGGTGTCATTACACATCAAGGT |
| | | GCCAATTCTGAGTCTGGACACTATCAAGCTTTCATAAGGGACGAACTGGACGAAAATAAA |
| | | TGGTACAAATTTAATGATGATAAAGTTAGCGTTGTTGAAAAGGAAAAAATTGAATCTTTA |
| | | GCCGGTGGGGGCGAAAGTGATAGTGCACTGATCTTAATGTATAAAGGATTTGGTCTGTAA |
| YGL012W | ERG4 | ATGGCAAAGGATAATAGTGAGAAGCTGCAGGTGCAGGGAGAGGAGAAAAAGTCCAAGCAA |
| | | CCGGTTAATTTCCTGCCTCAGGGTAAATGGCTGAAGCCAAATGAAATCGAATATGAGTTT |
| | | GGTGGGACTACTGGTGTTATTGGTATGCTGATCGGGTTTCCACTGCTAATGTACTATATG |
| | | TGGATTTGTGCGGAATTTTATCACGGTAAGGTTGCCCTACCCAAGGCTGGTGAATCGTGG |
| | | ATGCACTTTATCAAGCACCTATACCAGTTAGTCTTGGAGAACGGTATCCCAGAAAAGTAT |
| | | GACTGGACTATTTTCTTAACATTTTGGGTGTTTCAGATCATTTTCTACTATACGTTGCCC |
| | | GGGATTTGGACAAAAGGTCAACCATTGTCTCATTTGAAGGGAAAACAATTGCCTTACTTT |
| | | TGTAATGCCATGTGGACCTTGTATGTAACTACCACTTTGGTCTTGGTTTTGCACTTTACC |
| | | AATCTTTTTAGATTGTATGTCATTATTGACCGTTTTGGGAGGATCATGACATGTGCCATT |
| | | ATTTCAGGGTTTGCCTTCTCCATCATATTGTACTTATGGACTTTATTTATCTCACATGAC |
| | | TATCATAGAATGACAGGAAACCATCTATATGATTTCTTCATGGGAGCTCCACTAAACCCT |
| | | AGGTGGGGGATTTTGGACTTGAAGATGTTTTTCGAGGTTAGATTACCTTGGTTCACCCTT |
| | | TACTTTATCACTTTGGGTGCCTGTTTGAAGCAGTGGGAGACTTACGGCTATGTGACACCA |
| | | CAATTGGGGGTTGTCATGTTAGCTCATTGGTTGTACGCGAACGCATGTGCTAAAGGTGAA |
| | | GAATTGATTGTTCCAACCTGGGACATGGCTTACGAAAAGTTTGGATTTATGCTGATCTTC |
| | | TGGAATATTGCCGGTGTCCCATACACTTACTGTCATTGTACGTTGTATTTGTACTACCAT |
| | | GACCCATCTGAATATCACTGGTCTACACTGTACAATGTTTCGCTGTACGTTGTTCTATTA |
| | | TGCGCCTACTACTTCTTTGACACGGCAAATGCTCAGAAAAATGCCTTCAGAAAGCAAATG |
| | | TCTGGTGACAAGACAGGTAGGAAGACTTTCCCATTTTTGCCATACCAAATTTTGAAGAAT |
| | | CCAAAGTATATGGTTACCTCCAATGGATCGTACCTATTGATTGATGGTTGGTACACTTTG |
| | | GCTAGAAAAATTCACTACACTGCCGATTGGACTCAATCTCTCGTTTGGGCCTTGTCTTGC |
| | | GGGTTCAACTCGGTGTTCCCATGGTTTTTCCCAGTATTCTTCCTTGTTGTCCTGATTCAC |
| | | AGAGCCTTCAGAGACCAAGCAAAATGTAAGAGAAAGTACGGAAAAGATTGGGATGAGTAT |
| | | TGTAAACATTGCCCTTACGTCTTTATTCCTTATGTTTTCTAG |
| YGL038C* | OCH1 | ATGTCTAGGAAGTTGTCCCACCTGATCGCTACAAGGAAATCAAAAACAATAGTCGTAACC |
| | | GTACTTCTTATTTATTCTTTGTTGACATTTCACTTGTCAAACAAAAGGCTGCTTTCTCAG |
| | | TTTTACCCTAGCAAAGATGATTTCAAGCAAACTCTTCTCCCTACGACTTCTCATTCACAA |
| | | GATATAAATTTGAAGAAACAAATTACAGTTAACAAGAAAAAAAATCAATTGCATAATTTA |
| | | CGTGATCAATTATCGTTTGCGTTTCCCTACGACTCTCAGGCCCCCATCCCGCAAAGGGTG |
| | | TGGCAGACCTGGAAAGTCGGCGCAGATGATAAGAATTTTCCCTCTTCGTTCAGAACCTAT |
| | | CAAAAAACATGGTCTGGTTCGTATTCACCGGATTACCAATATTCTCTGATTTCGGATGAT |
| | | TCTATTATCCCTTTTTTGGAGAATCTTTACGCACCCGTTCCGATAGTCATCCAAGCGTTT |
| | | AAATTGATGCCTGGAAATATCCTAAAGGCAGATTTTTTAAGGTACCTATTATTATTTGCA |
| | | AGAGGTGGTATTTACTCAGATATGGATACTATGCTTTTGAAGCCAATTGATTCATGGCCT |
| | | TCTCAGAATAAGTCATGGCTAAACAACATAATAGATTTGAATAAACCTATTCCTTATAAG |
| | | AACTCAAAGCCCTCACTTCTCTCAAGTGATGAGATATCACACCAGCCAGGTTTGGTCATC |
| | | GGCATTGAGGCAGATCCGGACAGAGATGATTGGAGTGAATGGTATGCTCGTAGGATCCAG |
| | | TTTTGTCAATGGACTATCCAAGCCAAACCAGGTCACCCAATTCTAAGAGAATTGATCTTA |
| | | AATATTACTGCAACGACTTTGGCGAGCGTACAAAACCCAGGAGTTCCTGTCAGTGAAATG |
| | | ATTGATCCAAGATTTGAAGAAGACTACAACGTAAACTATAGGCACAAAAGACGTCATGAT |
| | | GAGACCTACAAACACTCCGAATTGAAAAATAACAAAAATGTTGATGGCTCAGATATAATG |
| | | AATTGGACGGGTCCAGGTATTTTCTCCGACATTATTTTCGAATACATGAACAATGTGCTC |
| | | CGATACAATAGCGATATCTTGTTAATCAACCCAAACCTAAACAAGAACGACGAAGAAGGT |
| | | AGTGAGAGTGCCACCACACCAGCAAAAGATGTTGATAACGATACGCTGTCCAAATCAACA |
| | | AGAAAATTTTATAAGAAAATATCAGAGTCTTTGCAATCGTCAAATTCAATGCCCTGGGAA |
| | | TTCTTCTCTTTTTTGAAAGAACCAGTTATCGTTGATGATGTGATGGTTCTACCAATAACA |
| | | AGTTTTTCACCAGATGTGGGGCAGATGGGCGCCCAGTCTAGTGACGACAAAATGGCTTTT |
| | | GTAAAGCACATGTTTAGCGGGAGCTGGAAAGAAGATGCTGATAAAAATGCAGGTCATAAA |
| | | TAA |
| YPL050C* | MNN9 | ATGTCACTTTCTCTTGTATCGTACCGCCTAAGAAAGAACCCGTGGGTTAACATTTTTCTA |
| | | CCTGTTTTGGCCATATTTCTAATATATATAATTTTTTTCCAGAGAGATCAATCTCTGTTG |
| | | GGACTTAATGGCCAGTCCATTTCCCAACACAAATGGGCACACGAAAAGGAAAACACATTT |
| | | TATTTTCCCTTCACCAAGAAATACAAAATGCCAAAGTACTCTTATAAGAAAAAAAGCGGC |
| | | TGGTTGTTCAACGATCACGTGGAAGATATTATCCCAGAAGGTCATATTGCACATTATGAT |
| | | TTGAACAAATTGCACTCTACGTCAGAAGCAGCTGTCAATAAGGAGCATATTTTGATATTG |
| | | ACTCCAATGCAAACATTTCATCAACAATACTGGGACAATTTGCTGCAGCTAAATTACCCT |
| | | CGGGAATTGATTGAATTGGGGTTCATTACACCAAGAACAGCCACTGGTGACTTGGCCTTA |
| | | AAGAAATTGGAGAATGCTATTAAAAAGGTTCAAACGGACAAGAAAACTCAAAGATTTAGT |
| | | AAAATTACTATTTTGCGACAGAATTCCCAGAGTTTTGATAAGTTGATGGAGAAGGAAAGA |
| | | CACGCTTTAGATGTTCAAAAGGAAAGACGTGCAGCAATGGCTTTGGCGCGCAATGAATTA |
| | | CTATTCTCCACCATAGGACCTCACACTTCTTGGGTGCTGTGGCTAGATGCCGATATTATA |
| | | GAGACACCACCATCTTTAATTCAAGACATGACCAAACACAACAAAGCTATCTTAGCTGCA |
| | | AACATTTATCAAAGATTTTACGATGAAGAGAAGAAGCAACCATCAATCAGACCATACGAT |
| | | TTCAACAACTGGCAAGAAAGTGACACCGGTTTAGAAATAGCCTCTCAGATGGGTGATGAC |
| | | GAGATTATTGTCGAGGGTTATGCAGAAATTGCCACTTATAGGCCATTAATGGCTCATTTC |
| | | TACGATGCTAATGGCGTACCAGGTGAAGAGATGGCGCTGGATGGTGTTGGTGGAGGCTGT |
| | | ACTTTGGTCAAAGCAGAAGTTCACAGAGACGGTGCCATGTTCCCTAATTTCCCATTTTAT |
| | | CACTTGATTGAAACAGAAGGTTTTGCTAAGATGGCGAAGAGATTAAACTACGATGTATTT |
| | | GGCTTACCAAACTATTTGGTTTATCACATAGAGGAAGAGAACCATTGA |
| YPL084W | BRO1 | ATGAAACCTTACTTATTTGACCTAAAGCTAAAAGACACAGAGAAGCTGGATTGGAAAAAA |
| | | GGCCTCTCCTCATATCTCAAGAAATCATACGGCTCCTCACAATGGAGAACATTTTATGAT |
| | | GAGAAAGCTACTTCAGAACTGGATCACTTAAGGAATAATGCAAATGGAGAACTAGCACCT |
| | | TCTTCATTGTCGGAACAAAATCTAAAATATTACTCATTTTTGGAGCATCTTTATTTTCGC |
| | | CTGGGTAGCAAAGGATCAAGATTAAAAATGGATTTCACTTGGTATGACGCAGAATACTCA |
| | | TCGGCCCAGAAAGGATTGAAATACACGCAACATACGTTAGCATTTGAAAAGTCTTGTACT |
| | | TTGTTCAACATTGCTGTAATCTTTACCCAAATTGCGAGGGAGAATATCAATGAGGACTAC |
| | | AAAAACTCAATCGCAAATTTGACAAAAGCTTTTTCCTGTTTTGAATATCTATCAGAAAAT |
| | | TTTTTGAACTCACCTTCAGTCGATCTTCAGTCAGAAAACACTAGGTTTCTGGCTAATATT |
| | | TGCCATGCAGAAGCTCAAGAATTGTTTGTCTTGAAATTATTAAATGATCAAATATCATCC |
| | | AAGCAATATACATTAATCAGTAAACTTTCTAGAGCCACGTGTAACCTCTTTCAGAAATGT |
| | | CACGATTTTATGAAAGAAATAGATGACGATGTGGCCATTTATGGTGAACCCAAATGGAAA |
| | | ACGACAGTTACTTGCAAACTGCATTTCTACAAATCGTTAAGCGCTTATTATCACGGTTTA |
| | | CACCTTGAAGAAGAAAATAGAGTTGGCGAAGCAATTGCTTTTCTCGATTTTTCTATGCAA |
| | | CAATTGATTTCATCCCTTCCATTCAAAACGTGGTTAGTGGAATTTATAGACTTTGATGGG |
| | | TTTAAAGAAACTTTAGAAAAGAAACAAAAGGAGTTGATTAAAGATAACGATTTTATATAT |
| | | CATGAAAGCGTTCCAGCCGTTGTGCAGGTTGATTCCATTAAGGCGCTCGATGCAATAAAA |
| | | TCTCCAACATGGGAGAAGATATTAGAACCATATATGCAAGATGTTGCAAATAAATGTGAC |
| | | TCTTTGTACAGAGGAATTATTCCCCTAGATGTCTATGAAAAGGAAAGTATTTACTCAGAA |
| | | GAAAAAGCGACGCTGTTGAGAAAGCAAGTTGAAGAAACTGAGACAGCAAATTTGGAATAT |
| | | TCTTCCTTCATCGAATTTACAAATCTACCCAGGCTCTTGAGTGATTTGGAAAAACAATTT |
| | | AGTGACGGAAATATTTTCTCGAATACGGATACACAGGGACAATTGATGAGGGACCAAATT |
| | | CAGACATGGTGTAAATTTATCCAAACAAATGAATTTAGGGATATAGAAGAACAGATGAAC |
| | | AAAATTGTTTTCAAAAGGAAACAAATTTTAGAAATCCTTTCTGCCTTACCCAATGATCAA |
| | | AAAGAAAATGTTACAAAACTAAAATCTTCTTTAGTAGCTGCTTCAAACTCAGACGAAAAA |
| | | TTGTTCGCATGCGTAAAACCACATATTGTCGAGATCAATCTATTGAATGACAATGGAAAA |
| | | ATATGGAAGAAGTTTGACGAATTTAATCGCAATACGCCTCCACAACCTAGCCTATTGGAT |
| | | ATCGATGATACCAAAAACGACAAGATATTAGAGTTGTTAAAACAAGTAAAGGGCCATGCG |
| | | GAAGACTTAAGAACATTGAAAGAGGAACGTTCGAGAAATTTGTCTGAACTAAGAGACGAA |
| | | ATCAACAACGATGATATCACAAAATTATTAATTATTAATAAGGGGAAATCCGATGTTGAG |
| | | CTCAAAGATTTATTCGAGGTGGAACTGGAGAAATTCGAGCCTTTGAGCACAAGAATAGAG |
| | | GCGACAATTTACAAACAATCTTCAATGATAGATGACATCAAAGCCAAGCTAGATGAAATT |
| | | TTTCACCTTTCAAATTTCAAGGATAAATCTTCTGGGGAAGAAAAATTTTTAGAAGATCGT |
| | | AAGAATTTTTTTGATAAGCTGCAAGAAGCAGTGAAATCATTCAGTATTTTTGCATCCGAC |
| | | TTGCCAAAAGGAATAGAGTTCTATGATTCATTATTCAATATGAGTAGAGACTTAGCAGAA |
| | | AGAGTGAGAGTTGCAAAGCAGACCGAGGATTCAACAGCTAATTCTCCCGCTCCTCCCCTC |
| | | CCTCCACTTGATTCTAAAGCGTCTGTCGTTGGGGGTCCTCCATTACTGCCCCAAAAAAGT |
| | | GCAGCCTTTCAGTCATTATCTAGACAAGGGCTCAATTTAGGGGACCAATTTCAAAATCTC |
| | | AAAATAAGTGCCGGCAGTGATTTACCTCAAGGACCCGGTATTCCACCAAGAACTTATGAA |
| | | GCTTCGCCATATGCTGCAACGCCTACTATGGCAGCCCCACCAGTACCACCGAAACAATCG |
| | | CAAGAGGATATGTACGACTTGAGAAGACGTAAAGCAGTTGAAAACGAAGAACGTGAACTG |
| | | CAAGAGAATCCTACGTCCTTTTACAATAGACCCTCTGTTTTTGATGAAAATATGTACTCC |
| | | AAATACAGCAGTTAG |
| YPL129W | TAF14 | ATGGTAGCTACAGTAAAAAGAACCATCCGTATAAAAACCCAACAACACATTCTGCCAGAA |
| | | GTGCCACCAGTAGAGAATTTTCCTGTTCGTCAATGGAGTATAGAAATTGTATTATTGGAT |
| | | GATGAAGGAAAAGAAATTCCCGCTACAATTTTTGACAAAGTTATTTACCACTTGCACCCA |
| | | ACTTTCGCCAATCCTAACAGGACATTTACTGACCCACCATTTAGAATTGAGGAACAAGGT |
| | | TGGGGTGGATTTCCTTTAGATATAAGCGTTTTCTTGTTGGAAAAAGCAGGTGAGCGGAAA |
| | | ATACCACACGATTTGAACTTTCTACAAGAGAGTTATGAAGTGGAGCACGTTATCCAGATT |
| | | CCTCTCAATAAACCACTTTTAACAGAAGAACTTGCGAAAAGTGGATCTACTGAAGAAACG |
| | | ACGGCCAATACAGGCACCATTGGGAAAAGAAGGACTACCACGAACACAACTGCAGAACCA |
| | | AAGGCGAAGAGAGCTAAGACGGGCAGTGCATCTACCGTGAAAGGGAGCGTGGACCTAGAA |
| | | AAATTAGCGTTCGGATTGACTAAACTAAATGAAGATGACCTGGTTGGTGTTGTTCAAATG |
| | | GTTACCGACAATAAAACACCAGAAATGAACGTGACGAATAATGTTGAAGAGGGTGAATTT |
| | | ATAATTGACTTGTATAGTTTACCTGAGGGATTATTGAAAAGTCTATGGGACTACGTTAAG |
| | | AAAAATACCGAGTAA |
| YPL254W | HFI1 | ATGTCAGCTATACAATCGCCAGCACCTAAACCTCTGCAGCCAACTTATCCAGCTGCTTCG |
| | | CCAGCTTCCACGAATGCATATATGAAGCCGGGCCTAATTGGTAGTCCTGCCGTCAGTAAT |
| | | CATACCGAGCCTAACAATGGTAACAATGAAACTGCGGAGCCTCAGGGACCAAATCAGAGA |
| | | ATTGATTTGGGTGCCATGATCGAAGAATTAACCTCACTATTGGGAAAAGAAAGCTGGACG |
| | | AAATATGCTCAGATCATCAGTCTTTTCATTTTAGGGAAGTTATCCAGAAAGGAACTTTCT |
| | | AATGAATTAGAGCTGGTATTTTCACCAAGCGCTGCAAGCTTAGAAAAATCAAATACAAAT |
| | | CATCACCATAGTTTAGTACGACTTCATAACCAACTTTTATTAGGGATTTTTGCTAATTCA |
| | | TTACGTGAAAACCCTCTTGGGAGAAACGGTAATGAGAGTTCTTGGGGATTTGGCAATGGA |
| | | AGCAACAATCCAAACAATAAACTAAAAAGAATCAATAAGCATAACTCTCAAATTGAAGTC |
| | | TATAAAAAAATTGTCATGTCGTTACCTCTAAATGATCGAAATAGACTTAAAATGATCACG |
| | | AAAGAGGCCGGCAAAAGAGGCTTCATTTTTTGCTCTGTATTTCAAGCCAGATTAAATAAT |
| | | ATACCCAAAATTCCCATTGTAACCAATCCAGAAAGTTTGAAGCGTGTCAAGAGCAATAAT |
| | | TTAAAAACGCCGCTGGAATGGTCACAAGATATAATGAATGGATTCAACGTTCCTTTAGCG |
| | | AGCGAAAGCCATTCTTTACCAGACACGGATTCGTTTTACTTAAGAATGGTTGGTATAGCA |
| | | AGAGAACATGGGTTAGTCGGCACAGTGGACGCACGTTGCGTAGAGCTTATATCATTGGCC |
| | | CTAGATCAATATCTAAAAAATATAATAGAGTTTACTATTGATACAGTTCGTTATAGAAGG |
| | | AAGAAATATTCAGATTATTATGATTTGAATGAGAGTGGGCTTTATAAATCCGTATCAGAA |
| | | ATGGCTGCTGATAAACGCGATGCCAAAATTAAGCAGTTAGATGATGATAAAAATGAGGAT |
| | | GAATGTGCCGATGAAGCCAAAAGTATTAATAACGGCAATAACAGTAGCAAGGACGATATT |
| | | GGCGACATATCAATGAGCAGCATTACAAAGGCTGGCGAAGCAGTTAATGAGGAGTTACAT |
| | | GAAAACAGAACGATATCATTAACGAATGAAGACATATATGATTCGTTATCTATATTTCCG |
| | | AATTTGGTTGAACCTTCAGGTTCATACTATGCGTTAACTAATTTAGGGCTAGTTAACGAT |
| | | GATGAACTAGTAGATATGAAGAGCAATATTGACGACTTGCCGGACTTCTTAAATGAAAAG |
| | | CCGACTTTCACGCCTTTGGATGAAAGAAACGTTGGTACAAGGCATGAATTAAATTGGTTA |
| | | ATTAAAGGAATCTTAACGGAAGATTGA |
| YPL268W | PLC1 | ATGACTGAAAGTGCTATAGATGACCAAAGGTTTAATCTTACAAAGGAGTTACAACGACAC |
| | | TCATGCAGAGATCAAGGGAAAATAACACAAAAGGATGATGCTTTAGATTTTATAAGTTAC |
| | | AGTTCTTTTCAATCCTCTTTTAATACTGACCAAAAAAGCGCGAACAATGGAAGTACTGTG |
| | | AGAAGAAGCATAAGGTCGATTTTTAGAAGAGCAGCCGAATTGCCTAGAGTCCATATGGGG |
| | | CCTCTTACTTATTCACATGGGATAAATGAGCTTGTTAACAAGAAATTAAGAAAAGACTGT |
| | | GATCTCAGCACGCTATGTCGCGTATTGCAAAGAGGAATCAGGATGATTAGGATGACAAGA |
| | | AGAAGAAGGAAGTTCTATGAATTTAAATTAATCAATAACAACGGGCAAATAATATGGAAA |
| | | GATGGTTCGAAGTATCTGGAATTAGACTCCGTTAAAGACATCAGAATCGGTGATACGGCG |
| | | AGTACTTATCAAGAGGAAGTAGATCCGAAAAGATTGAGATCGGACAGTAAACTTTGGATT |
| | | GCTATTATTTATAAGGTTTCCAACAAATTGAAAGCGTTACACGTAGTTGCTTTGAATGAA |
| | | TTGGACTTTAACACATTCTTAAGCTGCATTTGCGGTTTAGTGAAGTTAAGGAGGGAATTA |
| | | ATGGAAAGCATACTTTTGCCTGATAATTCGCAATTCGCTAGGATACATTGGCAAATTACT |
| | | GTTTCTGAAAAAGAGGAAGATGAGAAAAAGGACACATTGAGCTTTGCAGATGTGAAGAAA |
| | | CTATGTGATAAATTTCATATTTATGTGTCAACTGGTCAATTGTTAGAGTTTTTCCAATTA |
| | | GCAGACATCAATCATAACGGACTATTAAATTATTTTGAATTTGAAAAGTTCATTAAAATC |
| | | TTAAAGAATAGGAAAGAGGTAAATATGATTTGGTCTAAATTTACAAAACCTCCACACTCC |
| | | CACTTATCATTTGAAAATTTTTTTCAATTTTTAATTACAGAGCAGCATGAGCAAGTGGAT |
| | | CGTCAAACTGCATGGTCATATTTTATTAAGTACAGAGAACCGACTCAGCTGACGATGGGG |
| | | CAAGATGGTTTTACCAAGTTCTTGAAGGAGCAACCATATTTGGTGGAGGTTAAAGAGGAG |
| | | TTATATTCAAAGCCATTAAACCATTATTTTATTGCATCTTCACATAATACTTATTTATTG |
| | | GGGAAACAAATTGCAGAAACTCCATCTGTGGAGGGATATATTCAAGTCCTACAACAAGGT |
| | | TGTAGGTGCGTAGAAATTGATATATGGGATGGTGAAAATGGGCCAGTGGTATGCCATGGA |
| | | TTTTTAACGTCAGCTATTCCCTTAAAAACAGTTATACGCGTGATAAAGAAGTACGCTTTT |
| | | ATAACGTCTCCGTATCCTTTGATTATTTCACTGGAAATAAACTGTAACAAGGATAATCAA |
| | | AAATTGGCAAGCCTGATAATGAGAGAGGTTTTAGCAGAACAGCTGTACTTTGTTGGCACA |
| | | AGAACTGATAAACTGCCATCACCAAGGGAATTAAAACATAAAATACTGTTGAAATCCAAG |
| | | AAGACGAGTGAAGCAACTAGAGGTCTTAGCGTTAATGAGCCATTTCCATCTTCTTTTAGT |
| | | TCTTCTTACGAATCAGCTAATGAACAGGAATTAAGAATGAAAGATGACTCCACGAACAGT |
| | | AGTAGCGCAACAAATTCTTCTAGTATGCAACGCATCAAAAGAATTGGATTAAAAAAGCAC |
| | | GCTGATATAATTAATGATGTTTCTAACATTTCTGGAATACATGGCATCAAGTTCAGGAAT |
| | | TTCTCTTTACCAGAGTCCAAAACGATAGCGCATTGTTTTTCACTGAATGAGCGCAAAGTA |
| | | GAATACATGATAAAAGACAAACACTTAAAATTATCGTTGGATAAACATAATAGAAGATAT |
| | | TTGATGAGAGTCTATCCTCACGTTTTACGATACAAATCATCAAATTTTAACCCAATACCT |
| | | TTTTGGAAGGCCGGTGTACAAATGGTGGCTACGAATTGGCAAACTAATGATATTGGACAG |
| | | CAACTTAATCTGGCAATGTTTCAAATATTAGATCACCAACCTGATGGAAGTTTTAAATCA |
| | | GGATACGTGCTAAAACCAAAAAAGTTGCTACCTGTAGTAACAAAGGCGAAAATGATTCCA |
| | | TTAATTTACGAACATTTTGAAAATGGTAGTGATCCTGTTACTGTTAAAATTCGAATTTTA |
| | | TCTACACAGTTATTGCCCAGATTGAATGATACATCGCCAAGCAGAAATAACACCAATTCC |
| | | TTTGTTAAAGTGGAGTTTCATACCGATGACGAGCCGACAATGCCCATCTCAATCGATAAG |
| | | GGTACAAGGATTTCTGCCACTGAAGCATCCACTAAGAGTTCTCAAGGGAATGGATTCAAC |
| | | CCAATATGGGATGCTGAAGTTTCTATCACATTGAAAGATACAGACCTCACATTTATAAAA |
| | | TTCATGGTTATCTCTGAAGAGACCCAGATTGCATCTGTTTGCCTTAAGCTGAACTATTTG |
| | | AGGATGGGATATCGTCACATTCCGTTGTTTAACATGGAGGGGGAACAATACATATTTTGC |
| | | ACTTTATTTATTCATACACAAATTTTATAG |
| YGL058W | RAD6 | ATGTCCACACCAGCTAGAAGAAGGTTGATGAGAGATTTTAAACGTATGAAGGAAGATGCC |
| | | CCACCGGGTGTATCTGCTTCACCATTACCTGATAACGTCATGGTATGGAACGCCATGATT |
| | | ATCGGGCCAGCCGATACTCCATATGAAGACGGAACTTTTAGGTTATTGTTGGAGTTTGAT |
| | | GAAGAATATCCCAATAAGCCACCGCATGTCAAATTTTTGAGTGAAATGTTTCATCCCAAT |
| | | GTCTATGCAAATGGTGAAATTTGTTTGGATATTTTGCAGAACAGATGGACTCCAACATAT |
| | | GATGTCGCATCCATATTGACATCCATTCAAAGTTTATTCAACGATCCAAATCCAGCTTCG |
| | | CCAGCAAACGTTGAAGCTGCAACATTATTCAAAGATCATAAATCACAGTACGTCAAAAGA |
| | | GTTAAGGAGACGGTAGAGAAATCTTGGGAGGATGATATGGACGATATGGACGATGATGAT |
| | | GATGATGATGACGACGACGACGACGACGAAGCAGACTGA |
| YGL070C* | RPB9 | ATGACTACGTTTAGATTTTGTCGTGACTGCAACAATATGTTGTACCCTCGTGAGGATAAA |
| | | GAAAATAACCGGTTGTTATTTGAATGCAGAACATGCTCGTATGTAGAAGAAGCGGGCAGT |
| | | CCATTGGTCTACAGGCATGAATTGATTACCAACATTGGTGAAACTGCGGGAGTGGTGCAA |
| | | GATATTGGTTCCGATCCCACTCTTCCGAGATCTGATAGAGAATGTCCCAAATGTCACTCT |
| | | CGGGAAAATGTGTTTTTTCAATCACAACAAAGAAGAAAGGATACTTCGATGGTTTTATTC |
| | | TTTGTGTGCTTATCTTGCTCACACATATTTACTTCAGATCAAAAAAACAAAAGGACGCAG |
| | | TTTTCATGA |
| YGR006W | PRP18 | ATGGACCTAGATCTAGCCAGTATCTTAAAAGGTGAAATTTCTAAGAAGAAGAAAGAGCTG |
| | | GCCAACTCTAAAGGTGTTCAGCCACCATGCACTGAGAAATTCCAGCCACATGAATCCGCA |
| | | AACATAGACGAAACACCGCGACAGGTAGAACAAGAAAGTACAGATGAAGAAAACCTGTCA |
| | | GACAATCAGAGTGACGATATTAGGACCACCATTAGCAAATTAGAAAATCGGCCAGAAAGG |
| | | ATACAAGAAGCGATAGCTCAAGACAAAACCATCTCTGTAATCATTGATCCATCACAAATT |
| | | GGTTCTACTGAAGGAAAACCTCTTTTGTCAATGAAATGTAATTTATATATTCACGAAATA |
| | | TTATCTCGTTGGAAGGCGTCTTTGGAAGCTTATCATCCGGAGCTGTTCTTAGACACTAAA |
| | | AAAGCACTCTTTCCACTCCTACTGCAACTACGAAGGAACCAGCTTGCCCCGGATTTACTT |
| | | ATTTCCCTTGCGACCGTTCTCTATCATTTACAACAACCTAAAGAAATAAATTTAGCCGTT |
| | | CAGTCCTATATGAAACTCAGTATCGGCAATGTGGCCTGGCCTATTGGTGTTACTAGCGTA |
| | | GGCATTCATGCTCGTAGTGCACATTCGAAAATTCAAGGAGGCCGGAATGCTGCTAACATA |
| | | ATGATTGATGAAAGAACGCGATTATGGATTACCAGTATTAAAAGATTAATAACTTTTGAA |
| | | GAATGGTATACCAGCAACCACGATAGCTTAGCCTAA |
| YGR063C* | SPT4 | ATGTCTAGTGAAAGAGCCTGTATGCTGTGTGGCATAGTGCAGACCACAAATGAGTTTAAT |
| | | AGAGATGGTTGTCCCAACTGTCAGGGTATTTTTGAAGAGGCAGGTGTTTCTACAATGGAA |
| | | TGTACGTCGCCTTCTTTCGAGGGCCTCGTAGGAATGTGTAAGCCTACTAAGTCGTGGGTA |
| | | GCAAAGTGGCTGAGCGTAGATCATAGTATAGCTGGTATGTACGCCATCAAGGTCGATGGT |
| | | AGACTACCAGCTGAGGTTGTGGAGCTGTTGCCTCACTACAAACCGAGGGATGGCAGTCAA |
| | | GTTGAGTAA |
| YGR092W | DBF2 | ATGCTATCAAAATCAGAAAAAAATGTCGACCTCTTGGCAGGCAATATGAGCAATTTGAGT |
| | | TTCGATGGGCATGGGACTCCTGGGGGCACTGGACTATTTCCTAACCAAAATATAACAAAG |
| | | AGGAGGACAAGGCCTGCGGGTATCAATGATTCACCTTCGCCGGTAAAACCATCCTTTTTT |
| | | CCTTACGAAGATACTTCCAACATGGACATAGACGAAGTATCTCAACCTGATATGGATGTC |
| | | TCGAACTCTCCCAAGAAGCTTCCACCAAAGTTTTACGAAAGAGCAACTTCAAATAAAACA |
| | | CAGAGAGTTGTTAGTGTTTGCAAAATGTACTTTCTAGAACATTACTGTGATATGTTTGAC |
| | | TATGTAATTAGTAGAAGACAACGCACGAAGCAAGTTCTAGAATACCTGCAACAGCAAAGC |
| | | CAACTTCCGAATTCTGACCAGATTAAACTCAACGAAGAGTGGTCCTCTTACTTACAAAGG |
| | | GAACATCAGGTTTTGAGAAAAAGAAGGTTGAAACCAAAAAATAGGGATTTTGAAATGATC |
| | | ACACAAGTAGGTCAAGGTGGTTATGGGCAAGTTTATTTAGCCAGAAAGAAAGACACAAAA |
| | | GAGGTGTGTGCTTTGAAAATTTTGAACAAAAAACTATTGTTTAAGCTCAACGAGACAAAA |
| | | CACGTTTTAACTGAAAGAGATATCCTAACCACGACAAGGTCTGAATGGTTAGTAAAACTT |
| | | CTGTATGCATTCCAAGACCTACAAAGTTTATACCTGGCTATGGAATTTGTACCAGGCGGT |
| | | GATTTTCGTACATTATTAATAAATACTAGATGCTTGAAAAGTGGCCATGCGAGATTTTAC |
| | | ATCAGTGAAATGTTTTGCGCTGTTAATGCGTTACATGATTTAGGCTATACACATAGAGAT |
| | | TTAAAGCCGGAAAACTTCTTAATCGATGCCAAGGGACATATAAAATTAACAGATTTTGGT |
| | | TTGGCTGCTGGTACGATTTCTAATGAAAGAATTGAAAGTATGAAGATAAGGTTAGAAAAA |
| | | ATCAAGGACCTGGAATTTCCAGCGTTTACAGAGAAATCTATAGAAGATAGAAGAAAAATG |
| | | TACAACCAGTTGAGGGAGAAGGAAATTAATTATGCGAACTCCATGGTTGGGTCTCCGGAT |
| | | TATATGGCTTTGGAGGTTTTAGAAGGAAAGAAATATGACTTTACTGTGGATTACTGGTCG |
| | | TTGGGTTGTATGCTGTTCGAAAGTTTAGTTGGTTATACACCATTTAGTGGATCATCGACT |
| | | AATGAGACCTATGACAACTTAAGGCGCTGGAAACAAACTTTAAGAAGACCGAGACAATCT |
| | | GATGGGAGGGCAGCATTCTCCGATAGAACTTGGGATTTAATAACAAGATTGATTGCCGAT |
| | | CCAATCAATCGATTAAGATCCTTCGAACATGTTAAACGCATGTCTTATTTTGCAGATATA |
| | | AACTTTAGTACTTTGAGGTCAATGATCCCGCCTTTTACACCCCAACTAGACAGCGAAACT |
| | | GATGCCGGTTATTTTGATGACTTCACCAGTGAGGCTGACATGGCCAAATATGCTGATGTT |
| | | TTCAAAAGACAAGACAAATTAACGGCTATGGTAGATGACTCGGCAGTATCATCAAAACTT |
| | | GTTGGGTTCACTTTCCGACATAGAAATGGTAAACAGGGTTCCAGTGGCATCTTATTCAAC |
| | | GGACTGGAACACTCAGACCCCTTTTCAACCTTTTACTAG |
| YGR104C* | SRB5 | ATGGTTCAGCAACTAAGCCTTTTTGGATCTATTGGTGATGACGGCTACGATTTACTAATT |
| | | TCAACTTTGACCACAATATCAGGTAATCCTCCGCTACTGTATAACAGTTTATGCACTGTC |
| | | TGGAAACCAAATCCATCTTACGACGTCGAGAACGTGAACTCTAGAAACCAATTGGTTGAA |
| | | CCAAATAGAATAAAACTTTCCAAAGAGGTGCCATTTTCTTACCTGATCGATGAAACAATG |
| | | ATGGATAAGCCATTAAACTTTAGAATCTTGAAATCTTTTACAAACGATAAAATCCCGCTT |
| | | AACTATGCTATGACACGGAATATCTTGCACAACACAGTTCCGCAAGTCACCAACTTCAAC |
| | | AGCACAAACGAAGATCAAAACAACAGTAAGCATACAGAAGATACTGTAAATGAAAGTCGA |
| | | AACAGCGATGACATCATAGATGTCGACATGGATGCAAGTCCCGCCCCTTCAAACGAGTCA |
| | | TGTTCCCCTTGGTCATTGCAAATTTCAGATATTCCTGCTGCAGGAAACAATAGAAGTGTT |
| | | TCAATGCAAACGATAGCTGAGACTATCATATTATCTTCAGCTGGCAAAAACTCTTCAGTA |
| | | TCCTCGCTCATGAACGGATTGGGTTATGTATTCGAATTTCAGTATCTTACAATTGGTGTG |
| | | AAATTTTTTATGAAGCATGGTTTAATACTTGAGTTACAAAAAATTTGGCAAATAGAAGAA |
| | | GCAGGCAATTCACAAATAACAAGCGGAGGGTTCCTTTTAAAAGCATACATCAATGTTAGT |
| | | AGGGGGACCGATATCGATCGTATAAACTATACAGAGACTGCCTTGATGAACTTAAAAAAG |
| | | GAACTACAAGGCTATATAGAGTTAAGTGTACCCGATAGACAGTCAATGGACTCGAGGGTA |
| | | GCACATGGAAATATTCTAATATAA |
| YGR196C* | FYV8 | ATGAGCACTGAACAGGTCGGTAGAAAGAAATCGTATAGATGGGTTAGCGCATCTCAAGCA |
| | | AGTTATGACGGCGCTGGATGGGACAGTAGTGACGAATATGACTACTCTTCCGAGGATGGC |
| | | ACAAAAGGTTCAGAAATTCACAAGCAAAAAATATCTAATTTGCCTAGTTTACCCAAGCTC |
| | | AATTATACAGATGTTAATGGTGAGCATGATGAAAATACGGGTGAAAATAAAGACAGCAAT |
| | | GATAATAACGTCAGTAAGAGTGATATTAGTCCTAGTGACAAGGAAGTAGGCTATCTCTCA |
| | | GATGGGGTGCCTAAACTTATGGCTTCAAGAGAGTCTGTGGAATTACAAGCGAAAAAATCC |
| | | AGCGAGCACTCCAAATCAGATTATCTTTCGTCGACAGCATCTTTGAAAAGTCCAAGTGAA |
| | | AACAAAAAATCACCACATACAAATAGGGCTGTAAACGAAGACCTAGACAATTTGATAGAG |
| | | CAAATATCAAGGGAGATGACACCAGAAATTAGGCAAACATCTGATTTTCGGCGTGATTCA |
| | | GACAGTTGTGACGAAATCCAAAACGAAGCACCATTAGGGGAAGCGGTACCCTCTTCATCA |
| | | TCTCCTGTAGAAGAAGATGAAAAATCACATTCCTTAGGGGTTTCAATGGATACTAATGAA |
| | | GCTGATACCACTTTCAATACGCCAACAAGGAATGGAAATGAACATTTGAGTAGTGATGGC |
| | | GATGTTTCTGAGCAGAAAGACGACGAGTTTAAAGTTTCCGAAAGAGGCTACCTTGCAGAT |
| | | ATTCTGCCTGCGGAGAAAGAGGAGAATTTACAACAAGAAGATGATGGTGAAGTCGAGAGC |
| | | TCAGGAGCTTTGGAAAAGAAAGAAAAAAGTGAAGAAAAGACTAGTATTAGGAATAGGAAC |
| | | TCTACATCTAGTGGGCAAGATAAAGTAGCAAAGCCGAAACCCGTAGCAAATGAAACAAAA |
| | | ACTAGTGATAATGGATATCGAAATTCTTTTTTCAATGATTATCAGCATTCGTCAGACTCT |
| | | GAAGAAGATGATAACAACGAAGGCAACAGTGGTAGTAGTGATGATGATAATCGATCTTCG |
| | | GTTAGTGATAAACATGCTGATATTAACCGGCAATCGAAACAACTTGATACAACTGACGAT |
| | | GATGCATTGTCGTATACCGAATCAATAAAATATTCGACAAATGAGACAGAAGAAGAAGAT |
| | | AATGAAGATAATGAATCCATAGAAGACAAAAACGAAGATAATGAATCCATAGAAGACGAA |
| | | AATGAAGATACAGATAGCTATAAATTTTCTAATAGGGAAAAAGGTTCCATTCTACTTACG |
| | | AGTGATGAAGAGGAGGAGGAAAAAGGTATGAGCTCTGATTCAGATGAAGGCAGTCTCAAG |
| | | GCGCCCAAATCAGGTTATTTCTCCAAAATGATTGGTAATGATGATAAAGGTGATTCAGCA |
| | | TTGCAACCTAATCAAATAGACACCATTGAAAATACCAATTTGTCAAATAGCGGTTCTGAA |
| | | TTGGAAAACAGCGATGGCAGCGATGAGGAAGACCACATAAATGAAGACAAAGTTTTGGAA |
| | | GAAAGTTCAGTTAAAGACTCCACTGATGTTGATTCTTGGAAGCCGGACAGTGAGGCCTTA |
| | | CGATCAGGGTTCGTACAGGATACAGCCAATAAGAAAGCGCCACCAGGTTACGTGATCGAT |
| | | TCTAACGGGAAGCTAGTAGATTTAACACCTGCTAGTATGAAACCACGTGTTGTATCGACT |
| | | TATTCAGAGATGGAGAGTACTTGGGATGCATTCCCTTCCAAAGGCGAAGATGACGATTTG |
| | | GAGACAATCAGGGACACTAAGACTATATACGACAACAATACGATTTATAATGTTCCCGGT |
| | | TTGATTGGTAACCAATCGAATTTGCCACCTCTACCGATGGACGCACAGGAACAATTAAAT |
| | | GCAGGAAACGATAATAGTACAACAGATAATGATAATAGTAACAATACTGCCAATGATTTG |
| | | GCAGCGCGCAGTGCATCTTTCAAAAGTGAAAACAGAACAGTATCTCAAGGCGAAATGACA |
| | | AGCGTCCATGAACCCAGTACTGAAGAAATGGCGAAATTGGGGCAGCAGAATAACTTACCG |
| | | AAATTAGATATGAATAAACTACTGAACAGTAAGACTTCGCACGCCGGTAAGATAGAACAG |
| | | TTGAGAAATTATAAGCGTGAGCTAGATGAATATGATACGGGGATACAAACATGGATAAAT |
| | | TATACGCTAAAATCATCCTCTAATAAGGATAAGGATTTCATAGCTGAGGAGTATAAGCAG |
| | | CATAGTCACGTTAGGGAAGCATATGCGAACGCGGACGATTTGAGTAAGAAGCATACTGTC |
| | | ATTAACACGGTTGCTAGTGTCAACCAAAATGTAACGCACTTAAGGAGAAAAGTTTTCCAG |
| | | CATTCTATGAAACCAAAGGATTTATTCGCATCAATAGGTAAGAAAAAACTGTAA |
| YGR262C* | BUD32 | ATGACGCAAGAATTCATTGATAAAGTATCTTCCTACCTGACTCCTGATGTGGACATTGCA |
| | | CCTATTTCTCAAGGTGCAGAAGCCATTGTTTTCACAACGACTACTCACCCATATCTCCCA |
| | | AGAGCAAAGGATTCTCATCAAAAGTATATTATCAAATATAGGCCACCAAAGCGTTATAGA |
| | | CATCCACAAATAGACCAGGCACTAACAAAACATCGTACGTTGAATGAGTCGCGTTTATTG |
| | | GCCAAATTATACTTGATTCCAGGGCTATGTGTCCCTCAACTGATAGCGTGTGATCCATAC |
| | | AATGGATTCATTTGGTTAGAGTTCCTTGGAGAAGATCTTCCCGGAGGGCACGGTTTTAGT |
| | | AACCTAAAGAACTTCCTTTGGATGCATGACCAAGATCCATATAGTGATCTTGTAGCAACT |
| | | ACACTACGGAAAGTGGGGCGCCAAATTGGGTTGTTGCACTGGAATGACTACTGTCATGGT |
| | | GATTTGACAAGTTCTAACATTGTTCTCGTGCGAGATGGTGCGAGATGGACGCCTCATTTG |
| | | ATTGATTTCGGTCTGGGCTCAGTTTCAAACCTGGTCGAAGATAAAGGCGTCGATTTATAC |
| | | GTCTTAGAGAGAGCTATTTTAAGTACACATTCGAAGCATGCGGAAAAATACAATGCTTGG |
| | | ATCATGGAGGGGTTCGAAGAGGTCTATCGTGAACAAGGTGCGAAAGGTGCCAAGAAACTG |
| | | AAAGAAGTTACCAAAAGATTCGAAGAGGTCAGGTTGCGTGGTCGTAAGAGAAGTATGCTA |
| | | GGATAA |
| YGR271W | SLH1 | ATGTCAACTGAATACTCTGCTGATTCATCTAAATCATTTATGATTGCAATGCAATCTATG |
| | | ATTGATACTTCACAAACCTTTAATCTAGATAGATCGAAAATCTCTTTACCAGATTTTGAT |
| | | GACGAGCTAAAAAAAGTCCAAAAGGATGAACCAAATCAAAGAACTGAACTAACTGTTCTC |
| | | TCACAAGATAGAAATGATTGGGATGATATATTTGAGGAGTTTAAAGACATTTCATTTGCT |
| | | CAACTGCAATCTATAATTGATTCATACAAAACAAAGAATGCAGTTGCAGTATATAAAAAA |
| | | ATTGGAAAACTAATCAATGAAGCTGAAACGACACTAAGCAGCAATGTATTATTAGAAACT |
| | | GTACTGCAAATGGTTTATAAACATCAAAAGCAAGAGTTAGAGAAAGAATTGTTGGATTTT |
| | | TTAGGAACAGGGAATATAGACCTTGTTTCTCTGTTACTTCAGCATAGAAGAATGATCGTT |
| | | GCTACACCTATAGAAACGACGATTCTGCTAATAAAAAATGCGGTCAATTCAACGCCAGAG |
| | | TTCCTGACACAGCAAGATATCAGGAATCAAGTTTTGGAAAGTGCAGAGGATGCCAAGAAT |
| | | AGGAAATTAAATCCCGCCACTAAAATAATCAAGTACCCACACGTCTTCAGAAAATATGAA |
| | | GCAGGATCCACTACAGCTATGGCCTTTGCCGGTCAAAAATTCACTTTGCCAGTAGGGACT |
| | | ACAAGGATGTCGTACAATACCCATGAAGAAATTATAATTCCGGCAGCAGATCAGGCATCC |
| | | AACAAGAACTATCTCTATACAAAACTATTGAAAATAAGTGATTTGGATCATTTTTGCAAG |
| | | ACTGTATTTCCCTATGAAACTTTAAACCAAATACAATCCCTTGTTTACCCCGTAGCTTAT |
| | | AAAACAAATGAAAATATGCTAATTTGTGCACCTACTGGTGCAGGTAAAACAGACATTGCA |
| | | TTACTGACAATAATTAATACAATAAAACAATTCTCCGTGGTCAACGGGGAAAATGAAATT |
| | | GATATTCAATATGATGATTTTAAAGTTATTTATGTTGCCCCTTTGAAAGCACTCGCTGCT |
| | | GAAATCGTCGACAAGTTTAGTAAGAAATTGGCGCCTTTTAATATACAAGTAAGGGAGTTA |
| | | ACAGGCGATATGCAATTGACAAAGGCGGAAATTTTAGCCACTCAAGTAATAGTCACAACC |
| | | CCTGAGAAATGGGACGTTGTCACCCGTAAAGCAAACGGCGACAACGATTTGGTTTCAAAG |
| | | GTCAAATTACTAATTATTGATGAAGTTCATTTACTGCACGAAGATAGAGGTCCGGTTATT |
| | | GAAACTTTAGTTGCACGTACCTTGAGACAGGTAGAAAGCTCTCAATCAATGATTCGTATT |
| | | ATCGGTCTATCAGCTACATTACCCAATTTTATGGATGTAGCAGATTTCTTAGGAGTAAAT |
| | | AGACAGATCGGAATGTTCTATTTTGATCAATCATTCCGTCCAAAGCCTCTAGAGCAGCAA |
| | | CTTTTGGGCTGTAGAGGTAAGGCAGGTAGTAGACAAAGTAAAGAGAATATCGATAAAGTC |
| | | GCTTACGACAAATTGAGTGAAATGATTCAAAGAGGGTACCAGGTAATGGTATTTGTCCAC |
| | | TCAAGAAAGGAAACTGTTAAAAGTGCAAGAAACTTTATCAAACTAGCAGAATCTAACCAC |
| | | GAAGTTGACCTTTTTGCACCTGACCCTATCGAAAAGGATAAATATTCTAGAAGTTTAGTG |
| | | AAGAACAGAGACAAGGATATGAAGGAGATTTTTCAATTTGGTTTTGGTATCCATCATGCT |
| | | GGTATGGCCCGTTCAGATAGAAATCTTACTGAAAAAATGTTTAAAGACGGTGCTATCAAG |
| | | GTACTGTGTTGTACTGCAACATTAGCATGGGGTGTTAATTTACCTGCTGATTGTGTTATC |
| | | ATCAAGGGTACACAAGTATATGATTCTAAAAAGGGTGGATTCATAGATCTAGGTATTTCA |
| | | GATGTTATTCAGATTTTTGGCAGGGGTGGTAGGCCGGGCTTCGGTTCCGCAAATGGTACG |
| | | GGTATTTTATGTACTTCCAATGATCGTCTGGATCATTATGTTTCCTTAATTACTCAACAA |
| | | CACCCTATCGAATCTAGATTCGGTTCCAAGTTGGTTGATAACCTGAACGCCGAAATTTCA |
| | | TTGGGGAGCGTGACAAATGTAGATGAAGCAATCGAATGGCTTGGTTACACATACATGTTT |
| | | GTTCGTATGAGAAAAAATCCTTTTACTTACGGTATTGATTGGGAGGAAATTGCAAATGAC |
| | | CCGCAATTATATGAACGGAGGAGAAAAATGATCGTAGTAGCTGCAAGGCGATTACATGCT |
| | | CTCCAAATGATTGTGTTCGATGAAGTGTCAATGCATTTCATTGCAAAGGATTTGGGCAGG |
| | | GTGTCCTCTGATTTTTATCTTTTGAACGAATCCGTCGAGATATTCAATCAAATGTGTGAT |
| | | CCCAGAGCCACCGAAGCTGATGTATTGTCCATGATCAGTATGAGCAGTGAATTTGACGGT |
| | | ATAAAATTTAGAGAAGAAGAATCCAAGGAGTTGAAGAGACTTTCTGATGAATCTGTTGAG |
| | | TGCCAGATTGGAAGCCAACTTGACACCCCTCAAGGTAAAGCAAACGTGCTATTACAGGCT |
| | | TATATTTCCCAGACTAGAATTTTTGACTCAGCACTATCATCCGATTCGAATTATGTTGCT |
| | | CAGAACTCTGTTAGAATTTGTAGAGCATTGTTTTTAATAGGTGTCAATAGAAGATGGGGT |
| | | AAATTTTCAAACGTCATGCTAAATATTTGCAAATCTATTGAAAAGAGGCTCTGGGCCTTT |
| | | GATCACCCTCTCTGTCAGTTTGATTTGCCTGAAAATATTATCAGACGCATCAGAGATACT |
| | | AAACCTTCCATGGAGCATCTTTTAGAATTAGAGGCAGATGAGCTCGGAGAGCTGGTGCAT |
| | | AATAAGAAGGCCGGGTCTAGGTTATATAAAATATTGAGCCGATTTCCAAAGATTAATATA |
| | | GAAGCTGAGATTTTCCCCATTACAACAAATGTAATGAGGATACATATTGCATTAGGTCCA |
| | | GACTTTGTATGGGATTCCCGGATACATGGTGATGCACAGTTTTTTTGGGTTTTCGTTGAA |
| | | GAATCTGATAAGTCGCAAATCCTTCATTTTGAAAAGTTCATCTTAAACAGAAGGCAGTTG |
| | | AATAATCAGCATGAAATGGACTTCATGATCCCATTGTCAGACCCTTTGCCTCCGCAGGTT |
| | | GTCGTAAAGGTTGTGTCGGATACTTGGATTGGCTGTGAATCGACCCATGCCATCTCTTTT |
| | | CAACATTTAATCAGACCATTTAACGAAACTTTACAGACAAAATTGTTGAAGTTACGTCCT |
| | | CTACCTACATCAGCCCTCCAGAATCCTTTAATTGAGTCAATTTATCCTTTCAAGTATTTC |
| | | AATCCCATGCAAACTATGACATTTTATACACTTTACAATACCAATGAGAATGCGTTTGTT |
| | | GGCTCGCCTACAGGTTCTGGTAAAACGATTGTGGCTGAATTGGCTATATGGCATGCCTTC |
| | | AAAACATTTCCTGGGAAAAAGATTGTTTATATCGCACCAATGAAAGCATTAGTACGTGAA |
| | | AGGGTTGATGACTGGAGAAAGAAAATAACACCAGTGACCGGAGATAAAGTTGTAGAGTTA |
| | | ACCGGTGATTCTTTGCCAGATCCTAAAGATGTGCATGATGCCACCATTGTTATTACAACT |
| | | CCAGAAAAGTTTGATGGTATTTCCCGTAATTGGCAAACTCGTAAGTTTGTTCAAGATGTA |
| | | TCTTTAATTATCATGGATGAAATTCATTTGTTGGCTAGTGATCGTGGTCCTATCTTAGAA |
| | | ATGATTGTTAGTCGTATGAATTATATTTCCTCTCAAACCAAGCAACCGGTCAGACTGCTA |
| | | GGTATGTCAACTGCTGTTTCAAACGCCTACGACATGGCTGGCTGGTTGGGAGTTAAGGAC |
| | | CACGGACTGTATAACTTCCCGTCAAGCGTCCGTCCGGTTCCATTGAAAATGTACATTGAT |
| | | GGATTTCCAGATAATTTGGCATTCTGTCCTCTGATGAAAACAATGAACAAACCTGTGTTT |
| | | ATGGCCATTAAACAACATTCACCTGATAAACCAGCATTAATATTTGTTGCATCGCGTAGA |
| | | CAAACTAGACTAACGGCTTTAGACTTGATCCACTTATGTGGAATGGAAGATAATCCACGT |
| | | AGGTTTTTGAATATAGACGATGAGGAGGAACTGCAATATTACTTATCACAGGTCACTGAC |
| | | GACACTTTAAAGTTATCACTCCAGTTCGGTATTGGATTGCATCATGCAGGTCTAGTGCAA |
| | | AAAGATCGTTCTATTTCTCATCAGTTGTTTCAAAAAAACAAAATTCAGATTCTGATCGCT |
| | | ACATCAACCCTAGCGTGGGGTGTCAATTTGCCAGCTCACTTGGTTATCATCAAAGGTACA |
| | | CAATTTTTTGATGCAAAAATTGAAGGATATAGAGATATGGATTTAACAGATATCTTGCAA |
| | | ATGATGGGGAGAGCTGGTAGACCGGCTTATGACACTACAGGTACTGCTATCGTTTATACA |
| | | AAAGAGTCCAAAAAAATGTTTTATAAACATTTCCTAAACGTTGGTTTTCCTGTCGAGTCC |
| | | TCCTTGCACAAGGTATTAGATGATCATTTAGGTGCAGAAATAACGTCAGGTAGCATAACT |
| | | AATAAGCAAGAAGCGTTAGATTTTTTGAGTTGGACATTTTTGTTTCGTAGAGCGCATCAT |
| | | AACCCGACATATTATGGCATTGAAGATGATACAAGTACAGCAGGAGTCAGTGAACATTTG |
| | | AGTTCTTTGATAGATAGCACATTAGAAAATCTGCGAGAATCACAATGTGTTTTATTACAT |
| | | GGAGATGATATTGTTGCTACGCCATTCTTAAGTATATCCTCATACTACTATATTTCACAT |
| | | TTAACGATACGCCAATTATTGAAACAGATACATGATCATGCGACTTTTCAAGAGGTATTA |
| | | CGATGGTTATCGTTGGCTGTTGAGTATAATGAATTGCCTGTAAGAGGTGGTGAAATTATC |
| | | ATGAATGAAGAAATGTCGCAGCAATCGAGGTACTCCGTTGAAAGCACTTTTACCGATGAA |
| | | TTCGAATTACCGATGTGGGATCCACATGTAAAAACATTTTTATTACTTCAAGCTCATTTA |
| | | AGCCGTGTAGATTTACCGATTGCAGATTATATCCAGGATACCGTATCAGTTTTGGATCAA |
| | | TCTCTACGTATTCTACAGGCTTATATTGATGTCGCCAGTGAATTGGGTTACTTTCATACG |
| | | GTATTGACCATGATAAAGATGATGCAATGCATAAAGCAAGGGTATTGGTATGAAGACGAT |
| | | CCTGTTAGTGTACTTCCAGGATTACAACTGAGAAGAATCAAAGATTACACTTTTAGCGAG |
| | | CAGGGGTTCATTGAAATGACGCCGCAGCAAAAGAAGAAAAAGCTGTTAACCTTGGAGGAA |
| | | ATAGGAAGGTTTGGATATAAGAAATTATTAAATGTTTTTGATCAACTAACTTTTGGAATG |
| | | ACAGAAAGCGAAGATACGAAAAAACGTTTCGTCAGTGTCTGCCAAAGATTACCGGTTTTA |
| | | GAAGGCATGAAATTTGAAGAACAGGAAAATAACGAGGTGTTAACATTTTATAGCAAGCAC |
| | | CTTTCTAGTAAGCATAATAATGGGTTTGAAGTATATTGTGATAAATTCCCTAAGATACAA |
| | | AAAGAACTATGGTTTTTAATTGGACACAAAGGCGATGAACTGCTGATGATTAAAAGGTGT |
| | | CAACCAAAACAAATGAATAAAGAGGTCATAATACATTGTGATCTATTTATTCCCGAAGAA |
| | | ATTCGTGGCGAAGAATTGCAATTTTCCCTTATTAATGACGCTTTAGGATTACGTTATGAT |
| | | ATGGTCCATAAACTAATCTCTTAG |
| YHL025W | SNF6 | ATGGGTGTCATCAAGAAGAAAAGATCGCACCATGGAAAGGCTTCGCGCCAACAATACTAC |
| | | TCTGGGGTACAGGTTGGGGGAGTAGGCAGCATGGGCGCCATAAACAATAACATCCCGTCG |
| | | TTGACGAGCTTCGCGGAGGAAAACAACTATCAGTACGGATACAGCGGCTCCAGTGCCGGC |
| | | ATGAATGGCAGATCGCTTACGTACGCGCAGCAACAGCTTAATAAGCAAAGACAGGACTTC |
| | | GAACGTGTACGACTTAGACCAGAACAGCTCAGCAATATCATACATGACGAGAGCGACACG |
| | | ATATCGTTCCGATCCAACCTTTTGAAGAACTTTATAAGCTCGAACGACGCATTTAACATG |
| | | CTGAGTTTGACCACGGTACCGTGCGACAGAATTGAGAAGTCCAGATTGTTCAGTGAAAAA |
| | | ACAATAAGATATCTCATGCAAAAACAACACGAAATGAAAACACAAGCGGCAGAGTTGCAA |
| | | GAAAAGCCTCTGACGCCACTTAAATACACAAAACTTATAGCTGCGGCAGAGGACGGAAGC |
| | | CGTAGCACAAAGGATATGATAGATGCTGTCTTCGAGCAAGATAGTCATTTGAGGTACCAG |
| | | CCGGACGGCGTGGTCGTACATCGTGACGATCCTGCGCTGGTGGGTAAACTCCGCGGAGAT |
| | | CTCCGCGAAGCGCCGGCGGACTACTGGACGCATGCTTATAGGGATGTTTTGGCGCAATAC |
| | | CACGAGGCCAAGGAGCGTATCAGGCAGAAGGAAGTAACTGCAGGTGAAGCACAGGACGAA |
| | | GCCAGCTTGCAGCAGCAACAGCAGCAAGATTTGCAGCAACAGCAACAAGTAGTGACTACA |
| | | GTTGCCTCGCAAAGTCCTCATGCAACTGCAACGGAAAAGGAGCCAGTACCCGCCGTGGTT |
| | | GACGACCCACTGGAGAACATGTTCGGAGATTATTCCAATGAGCCGTTCAACACCAATTTC |
| | | GACGATGAATTTGGAGATCTTGATGCTGTATTTTTTTAA |
| YHR013C* | ARD1 | ATGCCTATTAATATTCGCAGAGCGACAATCAATGACATTATATGTATGCAAAATGCCAAC |
| | | CTTCATAACCTACCCGAAAATTATATGATGAAATATTATATGTATCATATTCTCTCGTGG |
| | | CCAGAGGCTTCGTTTGTTGCTACTACCACCACTTTGGACTGTGAAGATAGTGATGAACAA |
| | | GATGAGAACGACAAATTGGAATTGACCTTAGATGGGACCAATGACGGCAGGACAATCAAG |
| | | TTGGATCCAACATACTTGGCTCCGGGCGAAAAATTAGTTGGATACGTTCTCGTGAAGATG |
| | | AACGACGACCCCGACCAGCAAAACGAGCCACCAAATGGTCATATTACTTCCTTAAGTGTC |
| | | ATGAGAACCTATAGAAGGATGGGGATTGCCGAAAACTTAATGAGACAAGCTTTATTTGCG |
| | | TTGAGAGAAGTTCATCAAGCGGAATATGTTTCCTTGCATGTGAGACAATCTAATAGAGCA |
| | | GCTTTACACTTGTACAGGGACACATTGGCGTTTGAGGTGTTGAGTATCGAGAAAAGCTAC |
| | | TATCAGGATGGTGAAGATGCATACGCCATGAAGAAGGTTTTAAAATTAGAGGAGTTGCAG |
| | | ATAAGCAACTTCACCCATCGCCGTTTGAAAGAGAATGAGGAAAAACTAGAAGACGATCTA |
| | | GAAAGCGATCTACTAGAGGATATCATTAAGCAAGGCGTAAATGATATCATTGTATAA |
| YHR030C* | SLT2 | ATGGCTGATAAGATAGAGAGGCATACTTTCAAGGTCTTCAATCAAGATTTCAGTGTAGAT |
| | | AAGAGGTTTCAACTTATCAAAGAAATAGGGCATGGAGCATACGGCATAGTGTGTTCAGCG |
| | | CGGTTTGCAGAAGCTGCCGAAGATACCACAGTTGCCATCAAGAAAGTGACAAACGTTTTT |
| | | TCGAAGACCTTACTATGTAAAAGATCCCTACGTGAGCTAAAGCTTTTGAGACATTTCAGA |
| | | GGCCACAAAAATATTACATGTCTTTATGATATGGATATTGTTTTTTATCCAGACGGGTCT |
| | | ATCAATGGACTATATCTTTATGAGGAACTTATGGAATGTGATATGCACCAAATCATCAAA |
| | | TCCGGTCAACCTTTGACGGATGCTCACTATCAAAGTTTCACATACCAAATATTATGTGGT |
| | | TTAAAGTATATTCATTCTGCAGATGTCTTGCATCGTGATTTGAAGCCCGGCAATTTGCTT |
| | | GTCAATGCAGATTGTCAATTGAAAATCTGTGATTTTGGGTTAGCTAGAGGTTATTCGGAG |
| | | AATCCTGTCGAAAACAGTCAATTTTTGACGGAGTACGTGGCCACTAGATGGTATAGAGCT |
| | | CCGGAAATAATGTTGAGTTACCAAGGATATACCAAGGCGATTGACGTATGGTCAGCTGGC |
| | | TGTATTTTAGCGGAGTTTCTTGGTGGAAAGCCAATCTTCAAAGGAAAGGATTACGTTAAT |
| | | CAATTGAATCAAATATTACAAGTTTTAGGGACACCCCCAGACGAAACTTTAAGAAGGATT |
| | | GGTTCTAAAAATGTTCAGGACTACATACATCAATTAGGTTTCATTCCAAAAGTACCTTTT |
| | | GTCAATTTATACCCAAATGCCAATTCACAAGCATTAGACTTATTGGAGCAAATGCTCGCG |
| | | TTTGACCCTCAAAAGAGAATTACCGTGGATGAGGCCCTGGAGCATCCTTACTTGTCTATA |
| | | TGGCATGATCCAGCTGACGAACCTGTGTGTAGTGAAAAATTCGAATTTAGTTTTGAATCG |
| | | GTTAATGATATGGAGGACTTAAAACAAATGGTTATACAAGAAGTGCAAGATTTCAGGCTG |
| | | TTTGTGAGACAACCGCTATTAGAAGAGCAAAGGCAATTACAATTACAGCAGCAGCAACAG |
| | | CAGCAGCAACAGCAACAGCAACAGCAACAGCAGCCTTCAGATGTGGATAATGGCAACGCC |
| | | GCAGCGAGTGAAGAAAATTATCCAAAACAGATGGCCACGTCTAATTCTGTTGCGCCACAA |
| | | CAAGAATCATTTGGTATTCACTCCCAAAATTTGCCAAGGCATGATGCAGATTTCCCACCT |
| | | CGACCTCAAGAGAGTATGATGGAGATGAGACCTGCCACTGGAAATACCGCAGATATTCCG |
| | | CCTCAGAATGATAACGGCACGCTTCTAGACCTTGAAAAAGAGCTGGAGTTTGGATTAGAT |
| | | AGAAAATATTTTTAG |
| YJL028W | | ATGGCCCTGTGGGGTAGAAGTGCCTACAGACAAAAAACCGTTACGTCCCGCCTCACCAAG |
| | | CATCGACACACAAGCCCTCTCAACCTCCTTAATTTCTTTATCTTTTTTTCCCTTCACCTC |
| | | TGTGCTCTTTTTTTAGCCACAGCTGTGCATTACGCTTGCTTTGCTTGTTTTGTTCTCTTT |
| | | CGCCATGCCATATTACTACTTTTCTACTTACTGGCACGAGGCCGCGCAAGCCAGATCCAA |
| | | GCACGCCAGAAAGTGCGGTGTACTGGTGCAACCTTTTATCGATTTCTCATCATATCGCTA |
| | | TCGCAACGGGCATGGGCGACGAAAAAGCCAATCTAA |
| YJL095W | BCK1 | ATGCCCTTTTTGAGGAAAATAGCGGGGACAGCACATACACATTCTAGGTCTGATTCGAAC |
| | | TCATCTGTGAAATTCGGCCATCAGCCGACTAGTTCGGTAGCATCAACCAAAAGTTCAAGC |
| | | AAAAGCCCTCGTGCAACATCTCGCAAAAGCATTTATGATGATATTAGAAGCCAATTTCCC |
| | | AACCTAACCCCCAACTCTACCTCTTCTCAGTTTTACGAAAGCACGCCAGTTATCGAACAA |
| | | TCCTTTAATTGGACGACAGATGACCACATCTCAGCTGGAACGCTTGAAAACCCAACGAGC |
| | | TTTACAAACAGTTCTTATAAAAATGACAATGGACCTAGTAGCCTCTCTGATTCGAGGAAA |
| | | TCCTCCGGTGGCAATAGCGTAAATAGTTTGTCCTTTGACAAGCTAATTCTATCGTGGGAT |
| | | CCTACAGACCCTGATGAATGGACAATGCATCGCGTCACCTCATGGTTTAAATTTCATGAT |
| | | TTTCCAGAATCCTGGATATTGTTTTTCAAAAAGCATCAATTGTTTGGTCACAGATTTATA |
| | | AAGTTGCTTGCATATGATAATTTCGCTGTTTATGAAAAGTATTTGCCGCAGACTAAAACT |
| | | GCTTCATATACCAGGTTTCAGCAGTTATTGAAAAAAACAATGACCAAGAACGTAACAAAT |
| | | AGCCATATTCGTCAGAAGAGCGCTAGCAAACTTAAAAGTTCCAGGTCTTCCAGCGAATCG |
| | | ATCAAATCAAAATTAAAAAATAGTAAATCGCAAGAGGATATTTCAAATTCTAGATCAACG |
| | | TCAGAATCTGCATTGAGCCCAACAAAATCGGGCCCTTCCAAGACCGATGAAAAGAATTTT |
| | | TTACATTCTACTTCAACACACCAAAAAACCAAAAGCGCAAGTTCACTATACAGAAGAAGT |
| | | TTTATATCCCTAAGAGGCTCATCATCGAGCAATGCTTCCTCAGCAAAATCACCTTCAAAC |
| | | ATCAAGTTAAGTATACCGGCTCGGCCGCACTCAATTATTGAATCTAACAGTACACTTACC |
| | | AAATCGGCGAGCCCACCTGCATCTCCTTCGTATCCTAGCATATTTAGAAGACATCACAAA |
| | | AGTAGTTCATCTGAGTCGTCATTATTAAATTCCCTTTTTGGTAGTGGAATAGGCGAGGAA |
| | | GCTCCAACAAAGCCTAATCCACAAGGTCATAGTCTGTCTAGTGAAAATTTAGCTAAAGGA |
| | | AAATCTAAACACTATGAAACTAATGTGTCTTCACCTTTAAAACAATCTTCACTACCCACT |
| | | TCGGATGATAAAGGTAATTTATGGAATAAATTCAAAAGAAAGAGCCAAATAGGGGTTCCT |
| | | AGCCCAAATACGGTAGCTTATGTAACGTCTCAAGAAACTCCATCCTTAAAATCGAATTCG |
| | | AGTACTGCTACCTTAACCGTACAAACGGCAGATGTAAATATACCATCTCCATCTTCATCA |
| | | CCACCGCCAATACCCAAAACTGCAAACAGAAGTTTGGAGGTCATCAGCACAGAAGATACA |
| | | CCTAAAATTTCTTCAACCACGGCGTCTTTTAAAGAAACGTATCCTGATTGTATTAATCCA |
| | | GACAAGACAGTTCCAGTGCCGGTAAATAATCAAAAGTATAGTGTAAAGAACTTTTTACTG |
| | | GACCAAAAATTTTATCCTCTGAAGAAAACAGGGTTAAATGATAGTGAGAATAAATATATT |
| | | CTGGTTACCAAAGATAATGTTAGTTTTGTTCCGCTAAACTTAAAAAGTGTAGCAAAATTA |
| | | TCCAGTTTCAAAGAATCTGCTCTCACAAAATTGGGAATCAATCACAAAAATGTCACTTTC |
| | | CATATGACAGACTTTGATTGCGATATTGGTGCTGCAATTCCAGATGATACTTTGGAATTT |
| | | TTGAAAAAAAGCTTGTTTTTGAACACTTCTGGAAAAATTTATATCAAAGACCAAATGAAG |
| | | CTTCAACAAAAACCGAAACCTGCTCCTCTCACCTCAGAAAACAATGTTCCTTTAAAATCG |
| | | GTGAAAAGTAAGAGTTCAATGAGGTCCGGAACAAGCAGTCTGATAGCATCGACAGATGAT |
| | | GTTTCCATTGTCACTTCGTCTTCTGACATAACATCATTTGATGAACATGCATCAGGAAGT |
| | | GGGCGCAGGTACCCCCAAACCCCGAGTTATTACTATGACAGAGTTTCCAATACTAATCCA |
| | | ACTGAAGAATTGAATTATTGGAATATTAAAGAAGTTCTTTCTCATGAGGAAAATGCACCA |
| | | AAAATGGTTTTTAAAACAAGTCCAAAATTAGAACTCAACCTACCAGATAAAGGAAGTAAA |
| | | TTAAATATTCCTACCCCCATAACAGAAAATGAAAGCAAGAGTAGTTTTCAAGTGCTAAGA |
| | | AAAGATGAGGGGACTGAAATTGATTTCAATCATCGTAGGGAATCGCCTTATACAAAACCA |
| | | GAACTGGCACCAAAAAGAGAAGCTCCCAAGCCTCCCGCAAATACTTCTCCTCAGAGGACC |
| | | TTATCAACTTCTAAACAGAATAAACCGATCCGCCTAGTGAGGGCAAGTACAAAAATTTCG |
| | | AGAAGCAAAAGATCGAAACCATTGCCGCCACAATTATTATCATCTCCTATAGAAGCTAGC |
| | | AGCTCGTCTCCTGATTCGCTTACTTCCTCATATACTCCTGCTTCGACTCATGTTTTGATA |
| | | CCGCAACCTTATAAGGGTGCAAACGATGTTATGCGTAGGTTGAAAACAGACCAGGACTCG |
| | | ACGAGTACTTCCCCATCTTTGAAAATGAAACAGAAAGTGAATCGCTCAAATTCAACTGTA |
| | | TCGACTTCAAATTCAATTTTCTATTCTCCTTCACCATTGTTAAAAAGAGGTAACTCAAAA |
| | | AGAGTTGTTTCGTCGACATCTGCGGCCGATATATTTGAAGAGAATGACATAACATTCGCG |
| | | GATGCTCCGCCGATGTTTGACAGCGATGATAGTGATGACGATTCTAGTTCATCCGATGAC |
| | | ATTATCTGGTCCAAGAAAAAAACAGCTCCTGAGACTAATAATGAAAACAAAAAGGATGAG |
| | | AAAAGCGATAACAGTTCTACGCATTCTGACGAAATATTCTATGATTCTCAAACGCAGGAC |
| | | AAAATGGAGAGAAAGATGACCTTTAGACCATCTCCGGAGGTCGTTTATCAAAATTTAGAG |
| | | AAATTCTTCCCAAGGGCTAACTTAGATAAGCCAATCACTGAAGGAATAGCTTCACCAACA |
| | | TCTCCGAAATCCTTAGACAGCCTACTTTCACCAAAGAATGTGGCTTCATCGAGAACTGAG |
| | | CCAAGCACTCCTTCCCGTCCCGTCCCTCCTGATAGCTCATACGAGTTCATACAGGATGGA |
| | | CTTAACGGTAAAAATAAACCATTGAATCAAGCTAAGACACCTAAAAGAACAAAAACCATA |
| | | AGAACCATTGCACATGAAGCTAGTTTAGCAAGAAAAAACTCTGTAAAACTAAAAAGACAG |
| | | AACACCAAAATGTGGGGTACAAGAATGGTCGAAGTGACCGAAAACCATATGGTGTCAATT |
| | | AATAAAGCCAAAAATTCGAAAGGTGAGTATAAGGAATTCGCCTGGATGAAGGGTGAAATG |
| | | ATAGGGAAGGGATCTTTCGGTGCTGTTTATTTATGTTTAAACGTTACTACAGGTGAGATG |
| | | ATGGCCGTTAAGCAGGTTGAGGTCCCCAAGTATAGCTCACAAAATGAAGCCATTCTAAGT |
| | | ACCGTGGAAGCATTAAGATCTGAAGTGTCCACGTTAAAAGATTTAGATCATCTTAATATT |
| | | GTTCAATACTTAGGTTTTGAGAATAAAAACAATATTTACAGTTTGTTTTTAGAATATGTT |
| | | GCTGGTGGCTCCGTGGGATCCTTGATTAGAATGTATGGAAGATTCGATGAACCGTTGATC |
| | | AAACATTTAACAACACAAGTATTAAAAGGATTGGCATACCTACACTCGAAAGGTATTCTC |
| | | CACAGGGATATGAAGGCAGACAACTTACTTTTGGATCAAGATGGTATCTGCAAAATCAGT |
| | | GACTTCGGAATTTCAAGAAAATCAAAGGACATATACTCTAATTCGGATATGACCATGCGA |
| | | GGAACAGTCTTCTGGATGGCTCCTGAAATGGTTGATACAAAGCAAGGCTACAGTGCAAAA |
| | | GTTGATATATGGTCTCTGGGATGCATCGTTCTGGAAATGTTTGCTGGTAAGCGCCCGTGG |
| | | TCCAACTTAGAAGTCGTCGCAGCCATGTTCAAAATTGGAAAGTCAAAATCGGCACCACCA |
| | | ATTCCTGAGGACACTTTACCATTGATATCGCAAATCGGACGAAATTTTCTGGACGCATGC |
| | | TTCGAGATAAATCCAGAGAAAAGGCCAACCGCTAACGAGCTTCTTTCTCATCCTTTTAGT |
| | | GAAGTAAATGAAACATTCAATTTCAAATCTACCAGACTCGCGAAGTTTATAAAGTCAAAT |
| | | GATAAGTTAAACTCTTCAAAATTAAGGATAACCTCTCAGGAGAATAAAACTGAATAG |
| YJL129C* | TRK1 | ATGCATTTTAGAAGAACGATGAGTAGAGTGCCCACATTGGCATCTCTTGAAATACGATAT |
| | | AAAAAATCTTTCGGCCATAAATTTCGTGATTTTATTGCTCTATGTGGTCACTATTTTGCT |
| | | CCAGTTAAAAAATATATCTTCCCCAGTTTTATCGCGGTTCACTACTTCTACACGATATCC |
| | | CTGACATTAATAACTTCAATCCTGCTATATCCCATTAAGAATACCAGATACATTGATACA |
| | | TTGTTTTTAGCAGCGGGCGCAGTTACACAAGGTGGCTTAAATACTGTGGATATCAACAAT |
| | | CTAAGCTTATACCAACAAATTGTTCTGTATATCGTATGCTGCATATCAACACCAATTGCA |
| | | GTTCATAGTTGCTTGGCATTTGTACGGCTTTACTGGTTTGAGCGCTACTTCGATGGTATT |
| | | AGAGACTCTTCTAGACGAAATTTTAAGATGAGAAGAACGAAAACAATCTTAGAAAGGGAA |
| | | CTAACAGCAAGAACCATGACCAAGAATAGAACAGGTACCCAAAGAACGTCTTATCCTAGG |
| | | AAACAAGCTAAAACAGATGATTTCCAAGAAAAATTGTTCAGCGGAGAAATGGTTAATAGA |
| | | GATGAGCAGGACTCAGTTCACAGCGACCAGAATTCTCATGACATTAGTAGGGACAGCAGC |
| | | AATAATAATACGAATCACAATGGTAGCAGTGGCAGTTTAGATGATTTCGTTAAGGAAGAC |
| | | GAAACGGATGACAATGGAGAATATCAGGAGAACAACTCCTACTCGACGGTAGGTAGTTCG |
| | | TCTAACACAGTTGCAGACGAAAGTTTAAATCAGAAGCCCAAGCCAAGCAGTCTTCGGTTT |
| | | GATGAGCCACACAGCAAACAAAGACCCGCAAGAGTTCCCTCAGAGAAATTTGCAAAAAGA |
| | | AGGGGTTCAAGAGATATTAGCCCAGCCGATATGTATCGATCCATTATGATGCTACAAGGT |
| | | AAGCATGAAGCAACTGCTGAAGATGAAGGTCCCCCTTTAGTCATCGGGTCCCCTGCGGAT |
| | | GGCACAAGATATAAAAGTAATGTCAATAAGCTAAAGAAGGCCACCGGCATAAATGGTAAC |
| | | AAAATCAAGATTCGAGATAAGGGAAATGAAAGTAACACTGATCAAAATTCCGTGTCAAGT |
| | | GAAGCAAACAGTACGGCGAGCGTTTCGGACGAAAGCTCGTTACACACAAATTTTGGTAAC |
| | | AAAGTACCTTCATTAAGAACAAATACTCATAGATCAAATTCGGGCCCGATAGCCATTACT |
| | | GATAACGCAGAAACAGACAAAAAGCATGGGCCATCAATTCAATTCGATATAACTAAACCT |
| | | CCTAGAAAAATTTCAAAAAGAGTTTCAACCTTCGATGATTTGAACCCAAAATCTTCCGTT |
| | | CTTTATCGAAAAAAAGCATCGAAGAAGTACCTCATGAAACATTTTCCTAAAGCGCGGCGA |
| | | ATACGGCAACAAATTAAGAGAAGGCTTTCTACTGGTTCAATTGAGAAAAACAGCAGTAAC |
| | | AATGTTTCAGATAGAAAACCTATTACTGATATGGATGATGATGATGATGACGATGACAAC |
| | | GACGGCGATAACAACGAAGAATACTTTGCTGACAACGAAAGCGGCGATGAAGATGAACGA |
| | | GTACAGCAGTCTGAACCACATTCTGATTCAGAACTCAAATCGCACCAACAACAGCAAGAA |
| | | AAACACCAACTGCAGCAGAACCTGCACCGCATGTATAAAACCAAATCATTTGATGATAAT |
| | | CGTTCAAGAGCAGTTCCTATGGAACGTTCCAGGACCATCGATATGGCAGAGGCTAAGGAT |
| | | CTAAATGAGCTCGCAAGGACGCCTGATTTTCAAAAAATGGTCTATCAAAATTGGAAAGCC |
| | | CATCATAGAAAAAAACCGAACTTTAGGAAGAGGGGATGGAATAACAAGATATTTGAACAT |
| | | GGTCCCTATGCATCTGACAGCGATCGCAATTATCCTGATAATAGTAATACTGGAAACAGT |
| | | ATTCTTCATTACGCAGAGTCTATTTTACATCATGATGGCTCTCATAAAAATGGAAGCGAA |
| | | GAAGCCTCTTCCGACTCTAATGAGAATATCTATTCCACGAATGGAGGAAGCGACCACAAT |
| | | GGTCTTAACAACTATCCTACTTACAACGACGATGAAGAAGGCTATTATGGTTTACATTTC |
| | | GATACCGATTATGACCTAGATCCTCGTCATGATTTATCTAAAGGCAGTGGTAAAACGTAT |
| | | CTATCATGGCAACCAACTATTGGACGTAACTCAAACTTCCTTGGATTAACAAGAGCCCAG |
| | | AAAGATGAATTAGGTGGTGTCGAGTACAGAGCAATCAAACTTTTATGCACCATATTGGTT |
| | | GTCTACTACGTTGGATGGCATATTGTTGCTTTTGTTATGTTAGTACCTTGGATTATTTTG |
| | | AAAAAGCATTATAGTGAAGTTGTTAGAGATGATGGTGTTTCACCTACATGGTGGGGATTT |
| | | TGGACAGCAATGAGTGCATTTAATGATTTAGGTTTGACATTAACTCCAAATTCAATGATG |
| | | TCGTTTAACAAAGCTGTATACCCATTGATCGTTATGATTTGGTTTATCATTATCGGAAAT |
| | | ACAGGGTTTCCCATCCTTCTTAGATGCATCATTTGGATAATGTTTAAAATTTCTCCTGAT |
| | | TTATCACAGATGAGAGAAAGTTTAGGTTTTCTCTTAGACCATCCACGTCGTTGTTTCACC |
| | | TTGCTATTTCCTAAGGCAGCTACATGGTGGCTACTTTTAACGCTTGCAGGATTGAATATA |
| | | ACTGATTGGATTTTATTTATTATTCTAGATTTTGGCTCAACAGTTGTGAAATCATTATCG |
| | | AAAGGCTATAGAGTCCTTGTCGGCCTGTTTCAATCTGTTAGCACAAGAACTGCTGGATTC |
| | | AGCGTTGTCGATTTAAGTCAACTGCATCCTTCTATCCAAGTCTCCTATATGCTAATGATG |
| | | TATGTCTCCGTATTACCATTGGCCATCTCTATTCGACGGACAAATGTTTACGAGGAGCAA |
| | | TCTTTAGGACTATATGGAGATATGGGGGGAGAACCAGAAGATACGGATACTGAAGACGAT |
| | | GGTAACGATGAAGATGACGACGAGGAAAACGAGAGTCACGAAGGTCAAAGTAGTCAAAGA |
| | | AGTAGTTCGAACAACAACAACAATAACAACAGGAAAAAGAAAAAGAAAAAGAAAACTGAA |
| | | AATCCAAATGAAATATCTACAAAATCCTTTATCGGTGCCCATTTAAGGAAACAGCTTTCA |
| | | TTTGACTTGTGGTTTCTATTTTTAGGGTTATTTATCATTTGCATTTGTGAAGGGGACAAG |
| | | ATAAAGGACGTACAAGAACCAAACTTTAATATATTTGCAATTCTTTTTGAAATTGTTAGC |
| | | GCTTACGGTACAGTTGGGCTATCGCTAGGTTATCCGGACACCAACCAATCGTTTTCAAGA |
| | | CAGTTTACTACATTATCTAAGTTGGTGATCATAGCTATGCTGATCAGAGGCAAGAATAGA |
| | | GGTCTACCATACTCACTGGATCGTGCAATTATCTTGCCTAGTGATAGACTTGAACATATT |
| | | GACCACCTTGAGGGCATGAAATTGAAGAGACAGGCTAGAACCAATACAGAAGACCCAATG |
| | | ACGGAACATTTCAAGAGAAGTTTCACTGATGTGAAACATCGTTGGGGAGCTCTTAAGCGT |
| | | AAGACCACACATTCCCGAAATCCTAAAAGGAGCAGCACAACGCTCTAA |
| YJL175W | | ATGTTTTTAACGTCACCATTTGAATCCTGTATTGTGCTGTCTTCCTTAATAGCAGGCTTA |
| | | TTGTTTTCATTGTCTACCGGGTTCGTAGGTATACTAGGAGTGTTTGCCTCATTGTTCGAA |
| | | ACAGAACTCTCGGTTTCGCCAAAGAGATTATCACTATCCTCATTATCTTGGCCAAAGACG |
| | | TTCTGGGCCCTGCTTTCTTCTGTAGAAGGTGTTTCCTGGGAATCTAGCTTATTCGCTTGC |
| | | ATAGTCGGCTGCTGCTTTGCCGTTACCGTTATCGCCTCACTTTCTGCATCGCGTGTGTTC |
| | | GGAACAGTTGCTTCATCCTTCAGAGACTCATCTTGTTGCTGCGATTCTTCACCAGCAGTA |
| | | TCTGTATTGGCAACCCCTGCCACAGCTGCATTAGCGTTGCTATCACTATTGTTGTCATTG |
| | | CCATGTTGGTCCACGTCCACGGAAGCGTTTACCGTGGACCCCTCACCCAGTGTATTCTCC |
| | | ATGCTTGCAAACCGTATCACAATTGGTCTCTGA |
| YJL184W | GON7 | ATGAAACTACCGGTAGCACAGTACAGTGCACCAGATGGTGTGGAAAAAAGTTTTGCACCA |
| | | ATACGCGATGACCCTCGATACATGACCACAGAGGGAAGGACAACTGGGCCCAGTGACCAT |
| | | GTGCTAAACGCTGGCCAAATCGATAGAGACAAACCTTCAGAACCGGAACGCACGAAAGAT |
| | | GGCTCACAACTGACATACTTAGGCCAGCTGCGCACGCAGCTGACGGGGCTACAGGACGAT |
| | | ATTAACGAGTTTTTGACAGGAAGAATGGAATTGGCAAAAAATAAGAAGAAAGCCGGCGCA |
| | | GACGAGAAGCGGATCCAGGAAGAGATTAACCAGCTATTAGATGGTGGTGACGGTGACGAA |
| | | GATGCTGTTTAG |
| YJR090C* | GRR1 | ATGGATCAGGATAACAACAACCACAATGACAGCAATAGGCTGCACCCACCTGATATACAT |
| | | CCAAATTTGGGCCCTCAATTGTGGCTGAATAGTAGCGGTGATTTTGACGACAACAACAAC |
| | | AACAACAACAACAACAACAATAATAATAGCACAAGACCACAAATGCCATCACGAACTAGA |
| | | GAAACGGCAACTTCGGAAAGAAATGCAAGTGAGGTTAGGGATGCAACGCTAAATAATATC |
| | | TTTAGGTTCGATAGTATTCAACGGGAAACGCTTTTGCCAACCAACAACGGACAACCGCTA |
| | | AATCAAAACTTTTCGCTGACATTTCAACCACAACAGCAAACAAATGCGCTGAACGGGATT |
| | | GACATAAACACTGTGAACACAAACCTTATGAATGGTGTCAATGTTCAAATAGATCAACTT |
| | | AATCGATTGTTACCGAACCTACCAGAGGAAGAACGGAAGCAAATCCACGAATTCAAGCTA |
| | | ATAGTGGGCAAAAAAATCCAAGAGTTTCTGGTTGTTATAGAGAAACGTAGAAAAAAAATA |
| | | CTGAACGAAATTGAGCTAGACAACCTTAAACTAAAGGAGCTACGTATTGATAACTCCCCA |
| | | CAAGCAATTAGTTATTTGCATAAATTACAAAGAATGAGGCTTAGGGCGCTAGAGACAGAA |
| | | AACATGGAAATTAGAAATTTAAGGCTAAAAATATTAACAATTATAGAAGAGTACAAAAAG |
| | | TCATTATATGCATACTGCCATTCCAAGCTAAGAGGTCAACAAGTGGAAAATCCAACAGAT |
| | | AATTTCATCATTTGGATAAACTCCATAGATACTACTGAATCATCTGACTTGAAAGAAGGG |
| | | CTACAAGATCTTTCGAGATATTCAAGGCAGTTCATAAATAATGTGCTTTCGAATCCATCA |
| | | AATCAAAACATATGTACGAGTGTCACCCGAAGATCACCTGTGTTTGCCCTAAACATGCTA |
| | | CCCTCGGAAATATTACACTTAATATTAGATAAACTTAACCAAAAATATGATATTGTAAAA |
| | | TTCCTTACCGTTTCCAAACTCTGGGCTGAAATAATTGTGAAGATACTTTATTACAGACCG |
| | | CACATCAACAAAAAGAGTCAATTAGACTTGTTTTTAAGGACTATGAAGTTAACTTCTGAA |
| | | GAAACTGTATTCAACTATCGTTTAATGATCAAAAGATTAAATTTTTCATTTGTTGGTGAC |
| | | TACATGCACGATACAGAGCTTAACTATTTTGTCGGATGTAAGAATTTGGAGCGACTAACT |
| | | TTAGTATTTTGCAAGCATATAACCAGTGTTCCAATATCGGCTGTTTTGAGAGGATGTAAA |
| | | TTTCTCCAAAGTGTGGATATCACTGGAATAAGAGACGTTTCCGATGACGTATTTGATACC |
| | | TTAGCGACATATTGTCCCAGAGTACAGGGCTTTTATGTTCCTCAGGCAAGGAATGTAACA |
| | | TTCGATTCACTGCGGAATTTCATAGTCCATTCCCCGATGTTGAAAAGAATAAAAATCACA |
| | | GCAAACAATAACATGAATGACGAATTAGTAGAACTATTAGCCAACAAATGCCCTTTGCTT |
| | | GTAGAGGTCGATATAACATTAAGTCCAAATGTCACTGATTCTAGTTTGTTAAAACTCCTC |
| | | ACTAGGTTAGTTCAGCTGAGGGAATTCAGAATAACTCATAATACGAATATTACGGATAAT |
| | | CTTTTCCAGGAGCTTTCTAAAGTAGTTGACGATATGCCCTCTTTAAGATTGATTGATCTT |
| | | TCTGGATGTGAAAATATTACAGATAAAACTATAGAAAGTATAGTCAATTTAGCCCCTAAA |
| | | TTACGTAATGTTTTTCTAGGCAAGTGTAGCCGAATTACAGATGCATCGTTGTTCCAATTA |
| | | TCGAAGCTGGGCAAAAACTTGCAAACAGTGCATTTTGGGCACTGTTTCAATATAACTGAT |
| | | AACGGGGTAAGAGCACTCTTTCATTCATGTACAAGAATACAGTATGTGGACTTTGCGTGC |
| | | TGTACGAATTTAACCAATAGAACTCTTTATGAACTAGCAGACTTACCAAAATTAAAGAGA |
| | | ATTGGCCTTGTCAAATGTACGCAAATGACTGACGAGGGTTTGTTGAATATGGTTTCCTTG |
| | | CGAGGCCGAAATGATACTTTAGAAAGGGTACATTTATCTTACTGTTCTAATTTAACAATA |
| | | TATCCGATATATGAGCTTCTAATGTCTTGCCCAAGGCTCTCACATTTGTCTTTGACTGCT |
| | | GTTCCGTCATTTTTACGCCCCGATATAACGATGTATTGCAGGCCTGCACCCTCAGACTTT |
| | | AGTGAAAATCAACGTCAAATATTCTGCGTATTTTCAGGGAAAGGTGTTCATAAACTTCGC |
| | | CATTATTTAGTAAATTTAACGTCGCCCGCTTTTGGACCACATGTCGATGTAAATGATGTT |
| | | TTGACAAAATATATTAGATCCAAGAATTTGATATTTAACGGTGAAACACTTGAAGATGCT |
| | | CTTAGGAGAATCATAACTGATTTAAATCAAGATTCCGCTGCAATTATAGCTGCTACAGGA |
| | | TTAAATCAAATCAACGGTCTAAATAACGATTTTCTTTTCCAGAATATCAATTTTGAACGA |
| | | ATAGATGAAGTATTCAGTTGGTATCTCAATACTTTTGATGGCATTAGGATGAGCTCGGAG |
| | | GAAGTTAACTCACTATTATTGCAAGTAAACAAGACGTTTTGTGAAGATCCATTTAGTGAT |
| | | GTGGACGATGATCAAGATTATGTCGTAGCACCTGGTGTAAACCGGGAAATTAACAGTGAA |
| | | ATGTGTCATATTGTTAGAAAATTCCATGAGTTAAATGATCATATTGATGATTTCGAGGTG |
| | | AATGTTGCTAGTTTGGTAAGAGTTCAGTTTCAGTTTACTGGTTTTTTACTTCATGAAATG |
| | | ACTCAAACCTATATGCAAATGATTGAATTAAACAGACAAATTTGTTTAGTACAAAAAACG |
| | | GTTCAGGAATCGGGCAACATAGATTACCAAAAAGGGCTTTTAATATGGCGACTTTTATTC |
| | | ATTGACAAATTCATTATGGTGGTTCAGAAGTACAAGCTCTCCACCGTTGTTTTGAGACTA |
| | | TATTTAAAAGATAACATAACATTGTTAACCAGACAAAGAGAACTATTAATAGCCCACCAA |
| | | AGATCAGCATGGAATAACAATAATGACAATGACGCCAACCGGAACGCCAACAACATAGTG |
| | | AATATTGTATCGGATGCTGGGGCAAACGATACAAGTAACAATGAAACTAACAATGGTAAT |
| | | GATGACAATGAAACAGAAAATCCAAATTTCTGGCGTCAGTTTGGCAATAGAATGCAAATA |
| | | TCACCTGACCAGATGAGGAATCTCCAAATGGGACTTCGTAATCAGAACATGGTTAGGAAC |
| | | AATAACAACAACACAATTGACGAATCAATGCCTGACACTGCCATTGATTCTCAAATGGAT |
| | | GAAGCATCAGGAACGCCCGATGAAGATATGTTATAA |
| YJR102C* | VPS25 | ATGTCTGCATTACCTCCAGTATACTCATTCCCTCCACTATATACCCGTCAACCCAATTCT |
| | | TTAACAAGGAGACAACAGATAAGTACGTGGATAGACATCATATCACAATATTGCAAGACC |
| | | AAGAAAATTTGGTACATGTCTGTTGACGGGACTGTAATCAATGACAACGAACTAGATAGT |
| | | GGAAGTACAGATAATGACGATTCAAAAAAAATCTCGAAAAATCTTTTTAATAATGAGGAC |
| | | ATCCAAAGATCTGTATCACAGGTATTTATTGATGAAATTTGGTCGCAAATGACCAAAGAA |
| | | GGCAAATGCTTACCGATTGACCAAAGTGGTAGGAGAAGCAGCAATACCACCACTACGAGA |
| | | TATTTTATATTATGGAAGAGCTTAGACAGTTGGGCTTCTTTGATACTACAGTGGTTCGAA |
| | | GACTCTGGAAAACTGAATCAAGTCATTACGTTATACGAATTGAGTGAAGGTGATGAAACA |
| | | GTCAATTGGGAGTTCCATAGAATGCCCGAAAGCTTGCTCTATTATTGTTTGAAGCCCCTA |
| | | TGCGATAGAAATAGGGCCACAATGCTGAAAGATGAAAATGATAAAGTAATTGCCATTAAG |
| | | GTTGTTTAA |
| YKL002W | DID4 | ATGAGTTTGTTTGAGTGGGTATTTGGAAAGAATGTCACTCCGCAAGAGAGGTTAAAAAAA |
| | | AATCAAAGGGCTTTAGAAAGGACTCAGAGGGAGCTTGAAAGAGAAAAGAGAAAATTGGAA |
| | | CTACAAGATAAAAAACTTGTATCAGAAATTAAAAAATCGGCAAAGAATGGGCAAGTTGCA |
| | | GCTGCGAAAGTTCAAGCAAAAGATTTAGTAAGAACCCGGAATTACATCCAAAAATTTGAC |
| | | AACATGAAAGCTCAACTTCAGGCGATATCATTGAGAATACAGGCCGTTCGAAGTAGTGAC |
| | | CAAATGACACGTTCTATGAGCGAGGCCACTGGTCTTTTGGCTGGAATGAACAGAACAATG |
| | | AATCTACCTCAGTTGCAAAGGATATCGATGGAGTTTGAAAAGCAGAGTGATTTGATGGGT |
| | | CAACGACAGGAATTCATGGACGAAGCTATTGATAACGTCATGGGTGATGAGGTGGATGAA |
| | | GACGAAGAAGCTGACGAAATTGTAAATAAAGTTCTTGATGAGATTGGAGTGGATTTGAAT |
| | | TCACAGTTGCAAAGCACGCCTCAAAATCTGGTTTCTAATGCGCCGATCGCAGAAACAGCG |
| | | ATGGGGATTCCTGAACCTATTGGCGCCGGATCAGAATTTCACGGTAATCCTGACGATGAC |
| | | TTGCAAGCTCGGTTGAACACTTTGAAGAAGCAGACTTGA |
| YKL139W | CTK1 | ATGTCCTACAATAATGGCAATACTTATTCAAAGAGTTATAGCAGAAATAATAAGAGACCC |
| | | TTGTTCGGAAAGAGGTCGCCAAATCCTCAGTCCCTAGCGAGACCACCGCCACCAAAGAGA |
| | | ATACGGACTGATAGTGGTTACCAGTCGAATATGGACAATATATCTTCTCATAGGGTAAAT |
| | | TCAAATGACCAACCAGGCCACACGAAAAGTCGTGGTAACAATAATTTATCTCGCTATAAC |
| | | GATACATCCTTTCAAACAAGTTCCAGATATCAAGGTTCAAGATACAACAATAATAATACG |
| | | TCGTATGAAAATAGGCCTAAAAGCATAAAAAGGGATGAAACAAAGGCTGAATTCCTGTCT |
| | | CATTTGCCAAAGGGGCCCAAATCTGTTGAGAAATCAAGATATAATAATTCATCCAATACT |
| | | AGTAATGATATAAAAAATGGCTATCATGCTTCAAAATACTATAACCACAAAGGCCAGGAA |
| | | GGGCGGTCTGTCATAGCTAAGAAAGTTCCAGTTTCGGTTCTAACGCAGCAAAGGAGCACG |
| | | TCAGTTTACCTAAGGATAATGCAAGTCGGAGAAGGAACATATGGTAAAGTTTACAAGGCA |
| | | AAAAACACGAATACAGAGAAGTTAGTAGCGCTGAAAAAATTGAGATTACAAGGAGAGAGG |
| | | GAAGGCTTTCCCATAACCTCTATACGAGAGATTAAACTACTACAAAGTTTTGATCATCCA |
| | | AATGTCTCTACTATAAAAGAAATAATGGTCGAATCTCAAAAAACTGTATATATGATATTT |
| | | GAATATGCTGACAACGACTTAAGTGGATTACTATTAAATAAGGAAGTCCAAATTTCTCAC |
| | | TCACAATGCAAACATCTCTTCAAACAATTGCTATTAGGAATGGAATATTTACATGATAAT |
| | | AAGATTTTACACCGTGATGTTAAAGGCTCAAACATCTTAATCGATAACCAGGGGAATCTA |
| | | AAAATAACAGATTTTGGACTAGCGAGGAAAATGAATTCGCGAGCTGATTACACTAACCGT |
| | | GTCATTACGTTGTGGTACAGACCACCAGAACTATTGTTGGGAACTACAAATTATGGAACA |
| | | GAAGTTGACATGTGGGGTTGTGGCTGCCTCCTGGTGGAATTATTCAATAAAACTGCAATT |
| | | TTCCAGGGCTCTAATGAATTAGAGCAAATAGAATCAATTTTCAAGATTATGGGAACTCCT |
| | | ACAATAAATAGCTGGCCAACGCTTTACGATATGCCGTGGTTTTTTATGATTATGCCACAG |
| | | CAAACTACCAAGTATGTCAACAATTTTTCTGAGAAATTTAAAAGCGTTTTACCATCTTCA |
| | | AAATGTCTGCAATTGGCGATTAATTTGTTATGTTATGATCAGACGAAAAGGTTTAGTGCA |
| | | ACCGAAGCTTTACAAAGCGACTATTTCAAGGAAGAACCAAAGCCTGAACCTTTAGTTCTT |
| | | GATGGATTGGTAAGTTGCCACGAATATGAAGTCAAATTGGCAAGAAAACAAAAGCGTCCT |
| | | AACATACTATCCACCAACACAAACAATAAGGGTAATGGTAATAGTAATAATAATAATAAT |
| | | AATAATAATGACGATGATGATAAATAA |
| YKL213C* | DOA1 | ATGGGATATCAATTGAGTGCAACACTTAAAGGGCACGATCAGGACGTCAGGGATGTGGTA |
| | | GCTGTGGATGATTCAAAGGTTGCTAGTGTTTCGAGGGATGGAACAGTTCGCTTGTGGTCT |
| | | AAAGATGACCAATGGCTCGGTACGGTAGTTTATACAGGACAAGGATTTTTAAATAGTGTG |
| | | TGTTATGATTCCGAGAAGGAGTTGTTGCTTTTTGGAGGTAAGGATACTATGATCAACGGT |
| | | GTGCCCTTGTTTGCCACCTCAGGAGAGGATCCCTTGTATACCTTGATTGGCCACCAAGGG |
| | | AACGTTTGTAGTTTATCCTTCCAAGATGGTGTTGTGATAAGCGGTAGTTGGGACAAAACC |
| | | GCAAAGGTTTGGAAAGAAGGATCGTTGGTTTACAATTTGCAAGCACACAATGCCTCTGTA |
| | | TGGGATGCCAAAGTCGTATCCTTCTCAGAAAATAAATTCTTGACAGCTTCCGCCGATAAG |
| | | ACGATTAAACTGTGGCAAAACGATAAAGTGATCAAAACTTTCAGTGGGATTCACAATGAT |
| | | GTAGTAAGACATTTAGCTGTGGTGGATGATGGCCATTTTATCAGTTGTTCAAACGATGGC |
| | | CTCATCAAGTTGGTTGATATGCATACAGGTGACGTACTAAGAACATATGAAGGCCATGAA |
| | | AGCTTTGTTTATTGCATTAAACTGCTGCCCAATGGAGATATTGTAAGCTGCGGCGAAGAC |
| | | CGAACAGTCCGTATATGGTCAAAGGAAAATGGTTCGTTAAAGCAGGTAATTACACTTCCT |
| | | GCCATCTCTATTTGGTCCGTTGATTGTATGTCTAATGGTGATATTATTGTGGGAAGTAGT |
| | | GATAATCTTGTGAGAATATTTTCGCAAGAGAAGTCAAGATGGGCTTCCGAGGATGAAATA |
| | | AATGAACTATCTACGCAAGTGGAAAAATCCACTATAAGTTCCAAGACAATAGAATTTGAT |
| | | GAGTCTAAACTATCACCTTATGAAATTTTACAATCGCCGGGGCGCAAAGAGGGTCAAATT |
| | | GTGGTAGTCAAATCACCTCAAGGTACTATAGAAGCTCATCAATTTTCAAATTCTTCATGG |
| | | AAAAAGGTTGGTGATGTTGTCGGTGCAGGTGCTACTGGAAACGACAAAAAAATAGAATTC |
| | | GAGGGTAAAACGTATGACTACGTATTTGATGTGGATATTGAAGATGGAAAACCTCCCTTG |
| | | AAATTGCCGATTAATGTCAGCGACAACCCATATACAGCAGCCGACAATTTTTTGGCTCGT |
| | | TATGAACTGCCAATGAGTTATAGGGATCAAGTAGTACAGTTCATACTGAAGAACACTAAT |
| | | GGCATATCGCTAGACCAGCCCAACGATAATGCATCATCTTCTGCAGTATCACCTTCAAAA |
| | | ACGAGCGTAATGAAGGTTCTTCCTGTCAAGCAGTATCTTATCATGGAAAATTATAACCCA |
| | | GATACTATTTTCAATGGAATAGTAAAAATAAATTCAAATGAGAAGACCTTTGATGATGAA |
| | | ATTCTGGCGCAAATTGGTGGCGCACTACATGACATAGATGAAAGCTGGGAGTTGCTACTT |
| | | TCATTTGCTAACACTATAAGATCTAACTGGGAAATTAAGACTCCTGCATACGACATAGTA |
| | | CGATTGATTGTAAAAAAATTACCTTATTCTTCAGATATAAAAGATTACATCGAAGAAGGC |
| | | TTGGGCAATAAGAATATCACTCTAACCATGTTAACTGTGCGTATTTTGGTAAATTGCTTC |
| | | AATAATGAAAACTGGGGTGTGAAATTACTGGAATCAAATCAAGTTTATAAATCAATTTTT |
| | | GAGACTATAGATACTGAATTTTCTCAGGCATCTGCTAAACAGTCACAGAATTTGGCTATT |
| | | GCAGTTTCCACACTAATTTTCAACTATTCAGCTCTCGTCACGAAAGGGAACTCTGATTTA |
| | | GAACTTTTGCCTATCGTAGCTGACGCCATCAACACCAAGTATGGGCCATTGGAAGAGTAT |
| | | CAAGAATGCGAAGAAGCCGCATATAGGCTAACCGTAGCTTACGGAAATTTGGCTACTGTT |
| | | GAACCTACATTAAGACAATTTGCCAACTCCGTAACTTGGCTTGCAAACATCAAAAGGAGC |
| | | TATGGGAACGTGCCAAGGTTTAAGGATATTTTCGACGATCTCTCCTAA |
| YKR023W | | ATGACAAGAAAGCAGGCTATTGACTATGCAATCAAACAAGTTCCGCAAATATTGCCTTTG |
| | | GAAGAGTCTGATGTAAAAGCGCTGTGCGAACAAGTACTGAGTACATCATCTGATGATCCA |
| | | GAACAAATTGCTTCAAAATTTTTGGAGTTCCTAGGTCATGAAGATCTATCGTTTGAATTT |
| | | GTAATGAAGTTCAATGAATTATTGAATCAGAATGATAAAAAAGAGGAAAAAAAAACGAAA |
| | | AATGTGCATCTTGAACATACTGCCCCCACTTCATGGAAAAATGAATCAAAACAGCCCACT |
| | | AATAACTATATCAATAAGAAGGGAGATGAAAAACCAAAAAAATTAAAGGATGAGAAGAAA |
| | | AGCTCGACAACGAGGCCTACCGTTCAACCATCAAATCAATCTACGCAATCAAACCCCATA |
| | | AAGGAGAAAAAAGAGCACAGATCAAAGGGGAAATTACAATCGTTACAAGAAATAGACGAA |
| | | GCAATCAAGATGCTAGAGCTTCGAGATAGCGGTTCTTCTAAAAATTGTAATTGTCAAGGT |
| | | ACAAGGCATCCAGTTTTCGACATCGCGCCAAACTGTCTTCACTGTGGTAAAGTGGTTTGT |
| | | GTTATAGAGGGATTGAATAAAGGTAAATGCGGTCATTGCCATGAACAGTTGATTTCTGAT |
| | | AACGAAAGAACCCAAATGGTAGAAATTTTAAATCAGGAAAAAAACGAACTAAACGGTTCA |
| | | TCATCGTCCTTATCCAATGCGTCAAATGGAGCTAATGTCCCAAAGAAGAAGACTAAAACC |
| | | TACAAAATAACTTCCGGTATGGGTAAAAATTTATTTGCTGAACAGGACAAATTATTTGAT |
| | | TTCATCGAAAGGAAAAGAGAAAGAGAAAGAAAGCGTAATGAAGTGTTGAAATTGCAGGAG |
| | | GAAAAAGAAGAGAGCGAAGCAAAGGAAAGACAAGCGAGCGAGCATGACCATAAAGCTGAG |
| | | GAAAACCCGGAGTTGTTAGCAGCACAAGAACGATTAGATAGACTTTTATATTTCCAAGAC |
| | | ACTTCGGCAGAAAGAACAAAAATTATTGATAATGCCAGCGATTTTGACATGAATCAGGAG |
| | | GTGGGTTTATGGGGCAGCGCAAGAGAAAGAGCTCTTGCGTTGAAGAAGCAACAACGTAAT |
| | | TTGAGAAAGTGGGAAAAAGTAGAAAAGGAACGGAATGGCAGGCGCGAGAAGTACGTTGTT |
| | | AGCATGAATATAGGTTCAAACGGTAAAGTTACTATGACAGAAGTCCCTAAGGATACAGAG |
| | | AACGTAATTGCAGGTTCAGATGATGACATCAGTGATATAAGTGACGAAGAAGATATCAGC |
| | | GACCTTAAACATATACACGCTCTGAAAAGTGAAATAAATACAACGAAATCCTTGGAGAAT |
| | | TTGCATTTACAGTCCAAAGCATGGGATTACGAACGTGATAAAAAACAATTCGATAGGCCT |
| | | ACGTATGTGAAAAAAAATTCAGATACTGCACAACAGAATAGAAAAACGGAAGAAAAGGCT |
| | | CACGACATGCAAGCTTATGATTTAAAGTCAAGAGTACAAGTGGACCAAAATGCAGACGCA |
| | | TCAGTAGAACAGAATATTCTTGCTGTACTTTGA |
| YKR082W | NUP133 | ATGAGTGAAAAAAAAGTACATCTTCGTTTGCGGAAGGAACTTAGCGTACCCATTGCGGTG |
| | | GTTGAAAACGAATCCCTGGCGCAGTTGTCTTATGAAGAGGAAAGCCAGGCCTCTCTAATG |
| | | GACATTTCCATGGAGCAGCAACAGTTAAGGTTACATTCGCACTTTGATAATTCCAAGGTT |
| | | TTCACAGAAAATAACAGATACATAGTAAAAACCCTTCAAACAGACTACAGTAGCGGATTC |
| | | AGTAACGATGACGAATTGAATGGATATATCGATATGCAAATTGGATATGGACTAGTCAAT |
| | | GACCACAAGAAGGTTTACATCTGGAATATTCACTCCACTCAAAAGGATACGCCCTATATA |
| | | ACTGTACCATTTCGTTCTGATGATAATGATGAAATAGCAGTCGCGCCCAGGTGCATACTA |
| | | ACTTTCCCTGCTACAATGGATGAGTCTCCATTAGCACTGAATCCTAATGATCAGGACGAA |
| | | ACCGGAGGGCTTATCATTATCAAAGGTAGCAAAGCGATATATTATGAGGATATCAACTCC |
| | | ATAAATAATTTGAACTTTAAGTTATCTGAAAAATTCTCTCACGAGTTAGAATTACCCATC |
| | | AACTCTTCGGGCGGAGAGAAGTGTGACCTAATGTTAAACTGCGAACCTGCTGGTATAGTG |
| | | CTTTCTACAAATATGGGCAGAATATTTTTTATAACCATTAGAAATTCCATGGGTAAACCT |
| | | CAACTGAAGTTAGGCAAACTATTAAATAAACCTTTCAAATTGGGTATCTGGTCCAAAATT |
| | | TTCAATACAAACAGTTCAGTTGTCTCATTACGAAATGGACCTATCCTCGGTAAGGGGACA |
| | | AGGTTAGTATATATTACTACTAACAAAGGAATTTTTCAAACATGGCAGTTGTCTGCCACG |
| | | AATTCCCATCCAACAAAATTGATTGATGTAAACATTTACGAAGCCATTTTAGAGTCGCTC |
| | | CAGGATTTGTATCCATTTGCTCATGGTACGTTGAAGATTTGGGACTCTCATCCATTACAA |
| | | GATGAAAGTTCACAACTTTTCCTGTCATCTATTTACGACAGTTCATGCAATGAAACATAT |
| | | TATATTCTTTCCACGATTATCTTCGATTCTTCTTCTAATAGTTTTACTATTTTTTCCACT |
| | | TATAGATTGAACACTTTTATGGAGTCAATAACCGACACAAAGTTTAAGCCTAAGATATTT |
| | | ATACCTCAGATGGAGAATGCTAACGATACCAATGAGGTAACTTCCATTTTGGTAATGTTC |
| | | CCCAATGCTGTTGTCATTACTCAGGTGAACTCAAAATTGGACTCTAGCTATTCAATGAGA |
| | | AGAAAGTGGGAAGATATTGTTAGCCTTAGAAATGACATTGATATAATTGGTTCTGGTTAC |
| | | GACTCAAAATCTTTGTATGTTTTAACGAAACAAATGGGGGTGCTACAGTTTTTTGTGAAA |
| | | GAGAATGAGGAAACAAATTCTAAACCGGAAGTTGGGTTTGTCAAATCTCATGTTGATCAA |
| | | GCTGTTTATTTCTCCAAAATAAATGCAAATCCCATAGATTTCAATTTACCTCCAGAAATT |
| | | TCACTAGATCAAGAATCTATAGAGCATGATTTAAAATTAACCAGCGAGGAGATTTTTCAT |
| | | TCCAACGGCAAGTACATACCCCCTATGCTAAATACGTTGGGGCAACATCTCTCTGTCCGT |
| | | AAAGAATTTTTTCAAAATTTCCTGACATTTGTTGCTAAGAACTTCAACTATAAAATATCA |
| | | CCCGAACTGAAATTGGATCTGATCGAAAAATTTGAGATCTTGAATTGCTGTATTAAGTTC |
| | | AATAGTATCATCAGACAATCTGATGTATTGAACGATATATGGGAGAAGACTTTATCAAAC |
| | | TATAATCTGACACAAAATGAACACTTAACTACTAAAACAGTTGTAATCAACAGTCCCGAC |
| | | GTATTTCCAGTAATCTTTAAACAATTTTTAAACCATGTTGTGTTTGTTTTGTTCCCATCA |
| | | CAAAATCAAAATTTCAAACTAAATGTTACCAATTTAATCAACTTATGCTTTTATGATGGA |
| | | ATTCTTGAAGAAGGTGAAAAAACTATAAGGTATGAACTTTTGGAGTTAGACCCGATGGAG |
| | | GTTGATACATCCAAGCTACCATGGTTCATAAACTTTGATTACTTAAACTGTATCAATCAA |
| | | TGTTTTTTTGATTTTACATTTGCGTGTGAGGAGGAAGGAAGTCTGGATTCTTATAAAGAG |
| | | GGCTTGTTAAAGATTGTGAAAATTTTATATTATCAGTTCAACCAATTTAAAATATGGATC |
| | | AACACGCAACCCGTTAAATCTGTGAACGCCAATGATAATTTTATAAATATCAACAATCTT |
| | | TATGATGATAACCACCTGGATTGGAACCACGTCCTTTGTAAGGTAAATCTAAAAGAACAG |
| | | TGTATTCAAATAGCAGAATTTTACAAAGATTTATCTGGATTGGTGCAAACATTACAAACT |
| | | TTAGATCAAAATGACTCAACAACCGTATCGCTATACGAAACATTCTTCAACGAATTCCCT |
| | | AAAGAGTTTAGCTTTACATTATTTGAATATTTAATCAAGCATAAAAAATTGAACGACCTC |
| | | ATCTTCAGGTTCCCACAACAACACGATGTTTTAATACAGTTTTTCCAAGAGTCCGCTCCA |
| | | AAGTACGGTCATGTAGCATGGATCCAACAGATTCTGGATGGGTCGTACGCAGATGCCATG |
| | | AATACTTTAAAAAACATTACTGTTGATGATTCAAAGAAGGGCGAAAGCTTGAGCGAATGT |
| | | GAATTGCACTTGAATGTTGCAAAATTAAGCTCGTTGCTAGTTGAAAAGGATAATTTGGAC |
| | | ATTAATACTTTGAGAAAGATCCAATATAATTTAGATACAATTGATGCTGAGAAAAATATT |
| | | TCAAACAAATTGAAAAAGGGCGAAGTTCAGATATGTAAACGCTTCAAGAATGGCTCTATC |
| | | AGGGAAGTTTTCAACATATTAGTGGAAGAGCTTAAATCAACAACAGTAGTCAATCTATCG |
| | | GATCTCGTTGAGTTGTATTCTATGCTCGACGATGAAGAGAGCTTATTCATACCGCTAAGG |
| | | CTTCTTTCTGTTGATGGAAACTTACTGAATTTTGAAGTTAAAAAGTTCTTGAATGCTTTG |
| | | GTATGGAGAAGAATCGTTCTACTAAATGCTAGTAATGAAGGAGATAAACTGCTTCAGCAT |
| | | ATAGTCAAACGTGTTTTTGATGAAGAACTACCCAAAAATAATGATTTCCCCTTGCCCAGT |
| | | GTGGATCTTTTATGTGACAAATCGTTACTGACGCCAGAATACATAAGCGAAACATATGGC |
| | | AGGTTTCCTATTGATCAGAATGCCATACGTGAAGAGATATATGAAGAAATATCTCAAGTG |
| | | GAAACGTTGAACTCAGACAACTCACTCGAGATAAAGTTGCACTCGACTATTGGTTCTGTA |
| | | GCGAAAGAAAAAAACTATACCATCAACTATGAAACCAACACTGTAGAATACTAA |
| YKR093W | PTR2 | ATGCTCAACCATCCCAGCCAAGGCTCAGATGATGCTCAGGACGAAAAGCAAGGCGACTTC |
| | | CCGGTCATCGAAGAGGAGAAGACCCAGGCTGTAACGCTGAAGGATTCGTATGTTAGCGAC |
| | | GACGTCGCCAACTCCACGGAACGCTACAACTTGTCCCCTTCTCCGGAGGACGAAGACTTC |
| | | GAAGGCCCCACTGAAGAAGAAATGCAGACTTTAAGGCACGTTGGTGGTAAAATTCCTATG |
| | | AGGTGTTGGTTAATTGCTATTGTAGAGCTTTCCGAGAGATTCTCCTACTACGGGCTTTCC |
| | | GCACCATTCCAAAACTACATGGAATATGGACCTAATGACTCCCCAAAGGGTGTTCTGAGC |
| | | TTGAACAGTCAGGGTGCCACTGGGTTGTCGTATTTTTTCCAGTTTTGGTGTTACGTTACA |
| | | CCAGTTTTCGGTGGTTACGTTGCGGACACCTTCTGGGGTAAATATAATACAATTTGTTGC |
| | | GGTACCGCTATTTACATTGCCGGTATTTTCATTCTATTTATCACTTCGATTCCCTCCGTT |
| | | GGTAACAGAGACAGTGCTATTGGTGGGTTCATTGCTGCCATTATTCTGATCGGTATTGCC |
| | | ACTGGTATGATTAAAGCTAACCTTTCCGTGTTGATTGCCGACCAGCTTCCTAAGCGGAAA |
| | | CCCTCCATCAAAGTTTTAAAATCGGGCGAAAGAGTCATTGTCGATTCAAATATTACTTTA |
| | | CAAAACGTTTTTATGTTCTTCTATTTCATGATCAATGTCGGTTCTCTATCATTAATGGCC |
| | | ACTACTGAATTGGAATATCATAAGGGGTTCTGGGCGGCCTATCTATTGCCCTTCTGCTTC |
| | | TTTTGGATCGCTGTTGTCACTTTGATTTTTGGTAAAAAGCAATACATTCAAAGACCAATC |
| | | GGAGATAAAGTCATCGCTAAAAGTTTTAAAGTTTGTTGGATTTTAACTAAGAATAAGTTC |
| | | GACTTCAACGCTGCTAAACCTTCTGTTCATCCAGAAAAGAACTATCCATGGAATGACAAA |
| | | TTTGTTGATGAAATTAAGAGAGCTTTGGCGGCTTGTAAAGTCTTTATATTCTACCCAATT |
| | | TATTGGACCCAATACGGTACCATGATTTCCAGTTTCATCACTCAGGCCAGTATGATGGAA |
| | | TTACATGGAATTCCCAACGATTTCTTACAAGCATTCGATTCCATTGCGTTGATCATTTTC |
| | | ATCCCAATTTTTGAAAAATTCGTATATCCTTTCATTAGAAGATACACTCCACTAAAACCA |
| | | ATTACAAAAATTTTCTTCGGTTTCATGTTTGGATCTTTTGCCATGACATGGGCTGCTGTT |
| | | CTACAAAGTTTCGTTTACAAGGCTGGTCCATGGTATAATGAACCTCTGGGTCACAACACC |
| | | CCAAATCATGTCCACGTTTGCTGGCAAATACCCGCATATGTCTTGATTTCTTTTTCAGAG |
| | | ATCTTTGCCTCTATCACTGGGTTGGAATACGCTTATTCCAAAGCCCCAGCTTCCATGAAA |
| | | TCGTTTATTATGTCCATTTTCTTATTGACTAACGCCTTTGGTTCTGCAATCGGTTGTGCA |
| | | TTGTCCCCAGTGACCGTTGATCCTAAATTTACATGGTTATTCACTGGTTTGGCTGTTGCC |
| | | TGCTTTATTTCTGGTTGTTTGTTCTGGTTGTGCTTCAGGAAGTATAATGATACAGAGGAA |
| | | GAAATGAACGCTATGGACTACGAAGAAGAAGACGAATTTGATCTCAATCCAATTTCCGCA |
| | | CCTAAAGCTAACGATATTGAAATATTAGAACCAATGGAAAGTCTAAGATCCACCACCAAA |
| | | TATTAG |
| YLR025W | SNF7 | ATGTGGTCATCACTTTTTGGTTGGACATCAAGTAATGCCAAGAATAAAGAGTCACCAACA |
| | | AAGGCCATAGTGCGGTTGAGGGAGCATATCAACCTTCTATCCAAAAAGCAATCGCATTTA |
| | | CGTACTCAAATTACAAATCAAGAGAATGAAGCTAGAATCTTTTTGACGAAGGGCAATAAA |
| | | GTAATGGCGAAGAATGCACTTAAAAAGAAGAAGACAATCGAACAACTTTTAAGTAAGGTA |
| | | GAAGGCACAATGGAGTCTATGGAACAGCAGCTATTCTCTATAGAAAGTGCAAACTTAAAT |
| | | CTAGAGACAATGAGGGCTATGCAGGAAGGTGCTAAGGCAATGAAAACTATTCACAGTGGC |
| | | CTTGACATAGATAAAGTGGATGAAACTATGGACGAGATAAGGGAGCAAGTCGAATTAGGA |
| | | GATGAAATAAGCGACGCTATATCCAGGCCTTTAATTACTGGGGCAAACGAGGTGGATGAA |
| | | GATGAGCTGGACGAGGAATTGGACATGCTGGCTCAAGAAAATGCTAACCAAGAAACGTCC |
| | | AAGATCGTTAATAATAATGTTAATGCGGCGCCTATCTCAGAGAACAAAGTCTCACTACCT |
| | | AGTGTTCCAAGTAATAAAATTAAACAAAGTGAGAACTCTGTGAAGGACGGGGAAGAGGAA |
| | | GAGGATGAAGAAGATGAAGATGAAAAAGCATTAAGAGAACTACAAGCAGAAATGGGGCTT |
| | | TGA |
| YLR027C* | AAT2 | ATGTCTGCCACTCTGTTCAATAACATCGAATTGCTGCCCCCTGATGCCCTTTTTGGTATT |
| | | AAGCAAAGGTACGGGCAAGATCAACGTGCTACCAAGGTCGACTTGGGTATCGGGGCCTAC |
| | | AGAGACGACAACGGTAAACCATGGGTCTTGCCAAGTGTTAAAGCCGCCGAAAAGCTAATT |
| | | CATAACGACAGCTCCTACAACCATGAATACCTCGGTATTACCGGTCTGCCAAGTTTGACA |
| | | TCTAACGCCGCCAAGATCATCTTCGGTACGCAATCCGATGCCTTTCAGGAAGACAGAGTA |
| | | ATCTCAGTACAATCACTGTCTGGTACGGGTGCTCTTCATATATCTGCGAAGTTTTTTTCA |
| | | AAATTCTTCCCAGATAAACTGGTCTATTTGTCTAAGCCTACTTGGGCCAACCACATGGCC |
| | | ATTTTTGAGAATCAAGGCTTGAAAACGGCGACTTACCCTTACTGGGCCAACGAAACTAAG |
| | | TCTTTGGACCTAAACGGCTTTCTAAATGCTATTCAAAAAGCTCCAGAGGGCTCCATTTTC |
| | | GTTCTGCACTCTTGCGCCCATAACCCAACTGGTCTGGACCCTACTAGTGAACAATGGGTT |
| | | CAAATCGTTGATGCTATCGCCTCAAAGAACCACATCGCCTTATTTGACACCGCCTACCAA |
| | | GGGTTTGCCACTGGAGATTTGGACAAGGATGCCTATGCTGTGCGTCTAGGTGTGGAGAAG |
| | | CTTTCAACGGTCTCTCCCGTCTTTGTCTGTCAGTCCTTTGCCAAGAACGCCGGTATGTAC |
| | | GGTGAGCGTGTAGGTTGTTTCCATCTAGCACTTACAAAACAAGCTCAAAACAAAACTATA |
| | | AAGCCTGCTGTTACATCTCAATTGGCCAAAATCATTCGTAGTGAAGTGTCCAACCCACCC |
| | | GCCTACGGCGCTAAGATTGTCGCTAAACTGTTGGAAACGCCAGAATTAACGGAACAGTGG |
| | | CACAAGGATATGGTTACCATGTCCTCCAGAATTACGAAAATGAGGCACGCATTAAGAGAC |
| | | CATTTAGTCAAGTTGGGCACTCCTGGCAACTGGGATCATATAGTAAATCAATGCGGGATG |
| | | TTCTCCTTTACAGGATTGACTCCTCAAATGGTTAAACGACTTGAAGAAACCCACGCAGTT |
| | | TACTTGGTTGCCTCAGGTAGAGCTTCTATTGCTGGATTGAATCAAGGAAACGTGGAATAC |
| | | GTGGCTAAAGCCATTGATGAAGTGGTGCGCTTCTATACTATTGAAGCTAAATTGTAA |
| YLR226W | BUR2 | ATGTCTGCTACATCTTCAAGTGGTGACGTGAAAAAATTTCAAGCTGTACCAAAACCAACG |
| | | AGCAATGCATCACCACCACCAGCATCGAGTGGGTTTAACGCAAGGACCCTATGGCCGGAT |
| | | TTGATAGAAACACCAGAAAACCAGTGGGTTTTTGAGTGCAAAGATATTATTGAAAAGATA |
| | | GGCACTAATGGACCGATAGCAGTGGAAATAAAAAAAAATATGGAAAAATGCTTAATGTAC |
| | | TTTTACACACTTAAAAAAAAATTAAATCTTTTTGATCACACCTATACGGCATCATGCATT |
| | | CTATTTTACAGGTATTGGTTCATATATGGAATACCTACAGCCATAACTGAATGTATTCAT |
| | | ATATCACAGGGAATTCTGGTCACCGCTTGCAAAACAATGGAGAATAATAGGCCTATAGAG |
| | | GCCTATATTAAGGCGACTTGTGAGTTTTTGATGCAAAATATACCGTCTTTAAAAAGTCGA |
| | | ACAAATATTGATAAACTTAAATGGGAATTTAGAGATAAGTTAGTGACGAATGAAAAAAAA |
| | | ATACTATGTTTATTTGGCTTTGACTTAAATATCAGTAATCCAAAAGAATTGATTGAAGAA |
| | | GTGTTCAGTGGCTATTATAGATTCAACAGGGATCACAATCTGCCAGAAAACTTTAAAAAA |
| | | GCTTTTCCAAAAATTCTTCAAGAATCAAGAAATTTTATGGTCCAAGCTGTCACCCAGCCA |
| | | GTTTCTTTATTGTGCGACGGTTATACGTTCATTGTACTATCTCTCATATACTGTGGTTTA |
| | | GAGTATAAAAAGCTTGTAGACAAAGATTTCAGATATCCGAAAAACTTCTTCAAGGATAGA |
| | | TTCCCTATCGAAGTAACGCCGGAAAATTTTGCCAATATCTTTACCGATTACAAGTTACTG |
| | | GAAGAGAATTTTTTCAACTTAAAAAGTAATAAAGGTGCTAAATTACAAATTGATTCAAGC |
| | | ATGATTGATTCGGTGATTGACGAAAGTGGCGATGTGGAAAATGAAGTTTCAGAAATCTCT |
| | | GATCCGTTTAACTACGAGTTGATTAAATCCGGTGAGGTGAAGGAAGAGTTTTTGAATCAT |
| | | ATTGAAACAAGGGTTAAAGATTTACTAGATAAGGCTAAACAAGAAAGTATGAAGAGAAAG |
| | | GCTAAAGATCCTATAAGAACCCCAGATGCCAAAAAACCTAAAATATAA |
| YLR234W | TOP3 | ATGAAAGTGCTATGTGTCGCAGAGAAAAATTCTATAGCGAAGGCAGTTTCACAGATCCTA |
| | | GGAGGAGGCAGATCAACTTCAAGGGATTCCGGCTACATGTATGTAAAGAACTATGATTTC |
| | | ATGTTTAGTGGGTTCCCGTTTGCCAGAAATGGGGCTAACTGCGAAGTTACCATGACTAGT |
| | | GTTGCAGGGCACCTAACAGGCATTGATTTCAGCCATGATTCGCATGGGTGGGGAAAATGC |
| | | GCCATCCAAGAGTTATTTGATGCGCCACTGAACGAGATTATGAATAACAACCAAAAAAAG |
| | | ATAGCAAGCAACATCAAGCGAGAAGCGAGGAATGCAGACTATCTGATGATATGGACAGAT |
| | | TGCGACCGGGAAGGAGAGTACATCGGTTGGGAGATATGGCAGGAGGCCAAGAGAGGCAAC |
| | | AGGCTCATACAAAATGATCAAGTATACCGGGCAGTCTTTTCGCATCTCGAAAGACAACAC |
| | | ATATTAAATGCAGCACGAAACCCAAGTCGATTGGATATGAAGAGTGTGCACGCTGTAGGC |
| | | ACGCGGATTGAAATCGATCTTCGAGCAGGTGTTACATTCACCAGACTCTTAACAGAAACG |
| | | CTACGAAATAAACTGAGAAACCAAGCCACCATGACCAAGGATGGTGCAAAACACCGCGGT |
| | | GGTAACAAGAACGACTCACAAGTCGTATCGTATGGTACATGCCAGTTTCCAACGCTCGGC |
| | | TTTGTAGTAGACAGGTTTGAAAGAATACGAAATTTTGTTCCCGAAGAGTTCTGGTATATC |
| | | CAATTGGTAGTCGAAAACAAAGACAACGGCGGAACAACAACGTTCCAGTGGGACAGGGGC |
| | | CACTTGTTCGACCGGCTGAGCGTGTTAACGTTTTACGAGACATGCATCGAAACCGCCGGC |
| | | AATGTTGCTCAAGTAGTAGACTTGAAATCAAAGCCAACAACGAAATACAGACCTTTACCT |
| | | CTGACCACAGTGGAGCTACAAAAAAACTGCGCCCGGTACCTGCGTCTGAACGCCAAACAA |
| | | TCACTAGACGCAGCAGAAAAGCTATACCAAAAGGGGTTCATATCGTATCCAAGAACAGAG |
| | | ACTGATACTTTCCCACACGCAATGGACCTAAAATCCTTGGTCGAAAAGCAAGCTCAATTG |
| | | GACCAACTCGCTGCAGGCGGCAGAACCGCCTGGGCATCGTACGCGGCATCGCTGCTCCAA |
| | | CCCGAAAACACAAGTAACAATAACAAGTTCAAGTTTCCACGAAGCGGCTCCCATGACGAC |
| | | AAAGCGCATCCACCAATCCACCCCATCGTAAGTCTGGGGCCTGAAGCAAATGTTTCGCCA |
| | | GTGGAAAGAAGAGTATACGAGTACGTGGCCAGGCACTTTTTGGCATGCTGCTCAGAGGAC |
| | | GCCAAGGGCCAATCGATGACCCTTGTGTTGGACTGGGCCGTTGAACGTTTCTCAGCTTCA |
| | | GGTCTCGTAGTCCTAGAGAGAAATTTCCTCGATGTTTACCCTTGGGCCCGATGGGAAACC |
| | | ACCAAGCAGTTACCGCGGCTTGAAATGAATGCCCTCGTAGACATCGCGAAGGCCGAAATG |
| | | AAGGCGGGCACTACGGCGCCGCCCAAGCCGATGACTGAGAGTGAACTCATTCTCCTCATG |
| | | GATACAAACGGCATTGGCACAGACGCCACCATTGCGGAGCACATAGACAAGATCCAAGTA |
| | | CGTAATTACGTTAGGAGCGAGAAAGTAGGCAAGGAAACCTACTTACAACCCACGACCCTG |
| | | GGTGTCTCACTAGTGCACGGCTTCGAGGCCATCGGCCTCGAAGACTCCTTTGCAAAGCCC |
| | | TTCCAGCGCAGAGAAATGGAGCAAGACCTCAAGAAAATCTGCGAAGGTCATGCCTCCAAG |
| | | ACTGATGTTGTAAAGGACATAGTCGAGAAGTATAGGAAGTACTGGCACAAGACGAATGCC |
| | | TGCAAGAATACTCTCTTGCAAGTTTATGACCGTGTCAAGGCATCCATGTAA |
| YLR240W | VPS34 | ATGTCACTGAACAACATAACATTCTGTGTCTCACAGGATCTGGATGTTCCCCTGAAAGTG |
| | | AAAATCAAGTCATTGGAAGGACATAAGCCACTGTTGAAGCCATCTCAAAAAATCCTGAAC |
| | | CCTGAATTAATGCTGATAGGGTCAAATGTATTCCCTTCTAGTGATCTAATTGTATCCTTA |
| | | CAGGTATTTGATAAAGAGAGAAACAGAAATTTAACTCTTCCGATATACACGCCGTATATT |
| | | CCTTTCAGAAACTCTAGAACCTGGGATTATTGGTTGACTTTGCCCATACGTATCAAACAG |
| | | CTTACCTTTAGCAGTCATTTACGCATTATTTTGTGGGAATACAATGGATCCAAGCAAATT |
| | | CCCTTTTTCAATCTAGAAACGAGCATCTTTAACTTAAAAGACTGTACTTTAAAAAGAGGG |
| | | TTTGAATCCTTAAAATTTCGTTATGATGTGATTGACCACTGTGAAGTGGTCACTGATAAC |
| | | AAAGATCAAGAAAATTTGAATAAATATTTTCAGGGTGAATTCACGAGACTCCCTTGGCTT |
| | | GACGAAATCACTATAAGCAAATTAAGGAAACAACGGGAAAATAGGACTTGGCCTCAGGGC |
| | | ACCTTTGTCTTAAACTTAGAATTTCCAATGTTAGAGCTTCCTGTTGTGTTCATCGAAAGG |
| | | GAAATTATGAATACTCAAATGAATATTCCAACTTTGAAGAACAATCCTGGACTAAGCACA |
| | | GACTTACGAGAACCGAATAGGAATGATCCTCAAATCAAAATTTCTTTGGGGGACAAATAT |
| | | CACTCCACATTGAAGTTTTACGATCCTGACCAACCAAACAATGATCCAATAGAGGAAAAG |
| | | TATAGGAGATTGGAAAGAGCATCTAAAAATGCAAACTTGGACAAGCAAGTGAAGCCGGAC |
| | | ATTAAAAAGAGGGACTACTTGAATAAAATCATCAACTACCCCCCTGGCACTAAATTAACA |
| | | GCACATGAAAAGGGTTCAATATGGAAATATAGGTATTATTTGATGAACAATAAAAAAGCT |
| | | CTTACAAAGTTATTACAGAGTACAAATTTGAGAGAAGAATCGGAAAGAGTAGAGGTTTTA |
| | | GAACTAATGGACTCATGGGCAGAAATTGATATAGACGACGCATTAGAACTATTAGGCTCA |
| | | ACTTTTAAAAACCTTTCAGTGAGATCGTACGCCGTAAATAGGCTGAAGAAGGCATCAGAC |
| | | AAGGAGCTGGAATTGTACTTATTACAGTTGGTAGAAGCTGTCTGTTTTGAGAACCTGTCC |
| | | ACTTTTTCCGACAAATCTAACAGTGAATTCACTATTGTGGATGCCGTATCTTCGCAAAAG |
| | | CTTTCGGGTGATTCTATGTTACTATCTACATCGCATGCCAACCAAAAATTATTAAAGTCC |
| | | ATCTCGAGTGAATCGGAAACTTCCGGGACAGAATCGCTACCAATCGTCATTTCTCCCTTG |
| | | GCTGAGTTTTTGATTAGGAGAGCACTGGTCAACCCAAGGTTAGGAAGTTTTTTCTATTGG |
| | | TATTTAAAGTCCGAGTCTGAGGACAAGCCATATTTAGACCAGATTTTAAGTTCCTTTTGG |
| | | AGTAGACTGGATAAAAAATCTCGGAATATACTGAACGATCAAGTCAGGCTAATAAATGTG |
| | | CTCAGGGAATGTTGTGAAACAATTAAGAGGCTGAAAGATACTACAGCCAAAAAGATGGAA |
| | | TTACTAGTGCATTTATTGGAGACGAAAGTCAGGCCTCTTGTCAAAGTACGACCAATTGCT |
| | | TTACCACTAGACCCTGATGTGTTGATATGCGACGTTTGTCCTGAAACTTCGAAGGTATTT |
| | | AAAAGCTCTTTATCGCCACTAAAAATAACATTCAAAACGACCTTAAACCAACCATATCAC |
| | | TTAATGTTTAAGGTTGGCGATGATTTGAGACAAGATCAACTGGTAGTACAGATCATAAGT |
| | | TTAATGAATGAGTTATTAAAAAATGAAAACGTGGACTTGAAATTGACACCATACAAAATT |
| | | TTGGCAACAGGACCACAAGAAGGTGCCATTGAATTTATCCCTAATGACACATTAGCTAGC |
| | | ATACTAAGCAAATATCACGGTATTCTTGGCTACCTTAAACTCCACTATCCTGATGAGAAC |
| | | GCGACATTAGGCGTACAAGGCTGGGTTTTAGATAATTTTGTCAAATCTTGTGCTGGCTAT |
| | | TGTGTTATTACATACATCTTAGGTGTCGGCGATAGGCATTTAGACAACTTACTAGTCACG |
| | | CCAGATGGGCACTTTTTTCACGCAGACTTTGGTTATATCTTGGGTCAGGACCCCAAACCT |
| | | TTTCCGCCATTAATGAAATTGCCCCCTCAAATTATAGAGGCGTTTGGGGGTGCAGAATCA |
| | | TCAAATTATGATAAATTTCGCAGCTACTGTTTTGTGGCATATTCGATTTTAAGAAGGAAT |
| | | GCAGGCTTAATCTTAAACCTGTTTGAATTAATGAAGACTTCGAACATACCTGATATCAGA |
| | | ATAGATCCCAATGGTGCTATATTACGTGTAAGAGAGAGATTTAATTTGAATATGTCCGAA |
| | | GAAGATGCCACAGTGCATTTTCAGAATCTAATCAATGATAGTGTAAATGCTTTGCTGCCT |
| | | ATCGTGATTGATCATTTACATAATCTGGCACAATACTGGCGGACCTGA |
| YLR322W | VPS65 | ATGAGACACTGCATTATCTTTATAGTGTGTATCAGCATTGTTGAAATTAGAACCGTTCAT |
| | | ATTGAATTCATCAAAGAGATCGTTGTCATATTCCGCATAGTTGACCACTTTAGCCCTTTT |
| | | ATGCTGCCTTGTTTGTTGAGCCATTGTAAAGATGGGGATACCATCATCTTCGTTTGTCAA |
| | | TCTGTTATGAAAGTTAGAAATATAAGCTTGTGGAATAAGCTGGTTTTGGTGCGACATTGT |
| | | GTGCTGCTATGTGCCTTTCTTCTTTCTTTTTTCAATGTACTTCATTCAATAATCTCTATT |
| | | TGTCGTATTTTCTAA |
| YLR330W | CHS5 | ATGTCTTCAGTTGATGTACTGTTAACAGTAGGTAAGTTGGATGCCTCATTGGCGTTACTG |
| | | ACTACTCAGGATCATCATGTTATTGAGTTTCCTACAGTATTATTACCAGAAAATGTTAAA |
| | | GCTGGATCTATCATAAAAATGCAAGTTTCACAAAATTTAGAGGAGGAAAAAAAACAAAGG |
| | | AATCATTTTAAGAGTATACAAGCCAAAATTTTGGAGAAGTATGGTACCCATAAACCGGAG |
| | | AGCCCAGTTTTGAAAATTGTTAACGTTACGCAAACGAGCTGTGTTCTAGCATGGGATCCA |
| | | TTGAAACTTGGCTCAGCAAAATTGAAATCACTGATCCTTTATAGGAAGGGAATACGTTCA |
| | | ATGGTAATTCCAAATCCATTCAAAGTGACTACCACGAAAATATCCGGTCTTTCCGTTGAT |
| | | ACGCCATACGAATTTCAATTGAAACTGATAACCACGTCAGGAACATTATGGTCTGAAAAG |
| | | GTTATATTGCGTACACATAAGATGACTGACATGTCTGGTATCACTGTATGTTTGGGTCCA |
| | | TTGGATCCATTGAAAGAAATTTCAGACTTACAGATATCCCAATGTTTGTCTCACATCGGG |
| | | GCGAGACCTTTACAACGTCATGTTGCGATAGATACTACGCATTTTGTCTGTAACGATCTA |
| | | GACAATGAAGAAAGCAATGAAGAGCTTATAAGGGCAAAACATAACAACATACCAATTGTC |
| | | AGACCGGAATGGGTGAGAGCTTGTGAGGTTGAGAAAAGAATCGTTGGTGTTAGAGGATTT |
| | | TACTTAGATGCAGATCAAAGTATACTGAAAAACTACACATTCCCACCAGTTAATGAGGAA |
| | | GAACTTTCGTACTCAAAGGAGAATGAGCCGGTAGCCGAAGTAGCGGATGAAAATAAGATG |
| | | CCCGAGGACACAACAGATGTCGAACAGGTTGCATCACCTAATGACAATGAGAGTAATCCT |
| | | TCAGAAGCTAAGGAACAAGGAGAAAAGAGTGGACATGAAACTGCCCCAGTAAGTCCTGTA |
| | | GAAGATCCATTGCATGCTTCGACGGCTTTGGAGAATGAAACCACCATCGAAACCGTCAAC |
| | | CCCTCCGTAAGAAGTTTGAAAAGCGAACCTGTTGGTACTCCCAATATAGAGGAAAACAAA |
| | | GCGGACTCTTCCGCAGAAGCCGTGGTAGAAGAACCGAATGAAGCTGTGGCTGAAAGTTCT |
| | | CCAAATGAAGAAGCAACGGGACAGAAAAGTGAGGATACCGATACACATTCTAACGAACAA |
| | | GCTGATAATGGATTTGTACAGACTGAAGAAGTAGCTGAAAACAACATAACCACAGAAAGT |
| | | GCAGGGGAAAATAACGAACCTGCAGATGATGCAGCAATGGAATTTGGACGTCCAGAAGCT |
| | | GAAATTGAAACTCCAGAAGTAAATGAGTCTATAGAAGATGCCAATGAACCTGCGGAGGAT |
| | | TCCAATGAACCTGTGGAGGATTCCAACAAACCTGTGAAGGATTCCAACAAACCTGTGGAG |
| | | GATTCCAACAAACCTGTGGAGGATTCCAACAAACCTGTGGAGGATTCCAACAAACCTGTG |
| | | GAGGATGCCAATGAACCTGTGGAAGATACCAGTGAACCTGTGGAGGATGCCGGTGAACCC |
| | | GTACAAGAAACCAACGAGTTTACTACCGACATTGCCTCTCCAAGACATCAAGAAGAAGAT |
| | | ATAGAACTTGAAGCCGAACCTAAAGATGCTACCGAAAGTGTTGCAGTCGAGCCATCCAAT |
| | | GAAGATGTAAAACCAGAAGAAAAAGGTTCAGAGGCAGAAGACGATATCAACAACGTTTCC |
| | | AAGGAGGCTGCCTCTGGTGAGAGTACTACCCACCAAAAAACTGAGGCCTCTGCTTCTCTT |
| | | GAAAGCAGTGCCGTCACGGAAGAACAAGAGACAACGGAAGCCGAAGTAAATACAGATGAC |
| | | GTTTTGTCCACTAAAGAAGCTAAAAAAAATACTGGCAACAGCAACAGTAATAAGAAGAAG |
| | | AATAAGAAGAATAAGAAGAAAGGGAAAAAGAAATGA |
| YLR337C* | VRP1 | ATGGCAGGTGCTCCAGCTCCTCCTCCTCCACCGCCTCCACCAGCATTAGGTGGTAGTGCT |
| | | CCTAAACCGGCAAAATCTGTGATGCAGGGCCGTGACGCCCTGTTAGGTGACATTAGAAAG |
| | | GGAATGAAACTAAAGAAAGCTGAGACCAATGATAGAAGTGCCCCAATCGTGGGAGGTGGT |
| | | GTCGTTTCATCGGCCTCAGGAAGTTCTGGTACCGTCAGTAGTAAAGGACCCTCTATGTCA |
| | | GCTCCTCCAATCCCTGGGATGGGCGCTCCGCAGTTGGGCGATATTTTAGCTGGAGGTATA |
| | | CCGAAGTTAAAACATATCAATAACAATGCAAGCACTAAACCCTCTCCATCGGCGAGCGCA |
| | | CCGCCAATTCCGGGCGCGGTGCCATCTGTTGCGGCTCCCCCCATTCCTAATGCACCATTA |
| | | TCACCAGCACCCGCTGTTCCTAGTATTCCCTCGTCTTCTGCACCTCCAATTCCTGATATT |
| | | CCATCTTCTGCGGCCCCTCCTATTCCCATTGTACCATCATCTCCAGCACCACCGCTTCCA |
| | | TTATCAGGTGCCTCTGCTCCAAAGGTACCGCAAAATAGACCGCATATGCCTTCAGTAAGG |
| | | CCAGCACACAGAAGCCACCAAAGAAAAAGCTCTAATATTTCTCTTCCTAGTGTGTCAGCA |
| | | CCCCCTTTGCCTTCAGCGTCTCTGCCAACACATGTTAGCAACCCTCCACAAGCTCCACCC |
| | | CCTCCCCCAACTCCAACAATTGGCCTGGACTCAAAAAACATTAAACCTACAGATAACGCA |
| | | GTCTCACCACCATCTTCTGAAGTTCCTGCAGGCGGGTTGCCATTTTTGGCAGAAATTAAT |
| | | GCCAGGAGGTCTGAACGAGGAGCCGTCGAGGGCGTTTCTTCCACAAAGATTCAAACCGAA |
| | | AATCATAAATCTCCTTCACAACCACCACTTCCATCATCTGCTCCGCCCATTCCTACATCA |
| | | CATGCTCCGCCATTACCGCCTACTGCACCACCTCCACCATCGTTACCAAATGTGACCTCT |
| | | GCACCTAAAAAAGCAACTTCAGCTCCAGCTCCGCCTCCACCACCACTACCAGCAGCCATG |
| | | AGCTCTGCGTCGACTAACAGCGTAAAAGCAACCCCGGTTCCCCCAACTTTAGCACCACCG |
| | | TTACCAAATACTACATCAGTTCCTCCTAACAAAGCATCGTCAATGCCCGCGCCACCACCT |
| | | CCTCCTCCCCCACCACCTGGTGCTTTTTCAACATCCTCTGCCTTGTCAGCATCATCTATT |
| | | CCTTTAGCACCACTACCACCTCCTCCTCCTCCTTCGGTTGCAACGTCAGTACCATCGGCT |
| | | CCTCCTCCTCCTCCAACACTCACGACAAACAAACCCTCAGCGTCCAGTAAGCAATCCAAA |
| | | ATATCTTCATCCAGTTCTTCCTCGGCCGTAACACCTGGGGGACCCTTACCATTTCTTGCT |
| | | GAGATCCAAAAGAAGAGAGATGATAGATTCGTGGTTGGTGGTGATACTGGATACACTACC |
| | | CAAGATAAACAAGAGGATGTAATAGGTTCATCTAAGGATGATAACGTAAGACCTTCTCCT |
| | | ATTTCTCCATCTATAAACCCGCCTAAACAGAGTAGCCAAAATGGTATGTCGTTTCTGGAT |
| | | GAAATTGAATCCAAATTACATAAGCAAACATCATCGAATGCTTTTAACGCACCACCCCCC |
| | | CATACGGATGCTATGGCGCCTCCTTTGCCTCCTAGTGCACCTCCTCCTCCTATAACATCG |
| | | CTACCGACTCCAACTGCATCAGGTGATGACCACACCAATGATAAGAGTGAGACTGTGTTA |
| | | GGGATGAAAAAAGCAAAGGCCCCTGCACTGCCTGGACACGTTCCACCTCCACCAGTTCCA |
| | | CCAGTTTTATCAGATGATAGCAAGAATAATGTCCCCGCCGCTTCTTTATTACACGATGTT |
| | | CTTCCCTCCTCGAATCTCGAAAAACCTCCTTCTCCTCCTGTTGCAGCTGCTCCGCCATTG |
| | | CCAACATTTAGTGCACCAAGTCTGCCTCAGCAATCCGTGTCTACATCAATACCATCACCT |
| | | CCTCCTGTTGCGCCTACACTTTCCGTTCGTACCGAAACTGAATCAATTTCGAAGAACCCC |
| | | ACTAAGTCGCCACCCCCACCACCAAGTCCATCTACGATGGATACCGGTACCTCTAATAGC |
| | | CCCAGTAAAAACCTTAAACAACGGTTATTTTCTACAGGTGGATCGACGCTGCAACACAAG |
| | | CATAATACGCATACAAATCAACCAGATGTTGATGTAGGTAGGTATACGATAGGGGGATCG |
| | | AATTCTATCGTTGGGGCCAAATCTGGAAATGAAAGAATTGTTATAGATGATTCAAGATTT |
| | | AAATGGACTAATGTGTCACAAATGCCTAAGCCACGTCCCTTTCAAAATAAAACAAAACTG |
| | | TATCCAAGTGGAAAGGGTAGTAGTGTGCCATTGGACTTAACATTATTTACGTGA |
| YLR338W | OPI9 | ATGTTTTTTGAGTCCAGGCCAATTGTTGGAGTTGGGGGAGGGGGTGGAGCTTGTGGAGGG |
| | | TTGCTAACATGTGTTGGCAGAGACGCTGAAGGCAAAGGGGGTGCTGACACACTAGGAAGA |
| | | GAAATATTAGAGCTTTTTCTTTGGTGGCTTCTGTGTGCTGGCCTTACTGAAGGCATATGC |
| | | GGTCTATTTTGCGGTACCTTTGGAGCAGAGGCACCTGATAATGGAAGCGGTGGTGCTGGA |
| | | GATGATGGTACAATGGGAATAGGAGGGGCCGCAGAAGATGGAATATCAGGAATTGGAGGT |
| | | GCAGAAGACGAGGGAATACTAGGAACAGCGGGTGCTGGTGATAATGGTGCATTAGGAATG |
| | | GGGGGAGCCGCAACAGATGGCACCGCGCCCGGAATTGGCGGTGCGCTCGCCGATGGAGAG |
| | | GGTTTAGTGCTTGCATTGTTATTGATATGTTTTAACTTCGGTATACCTCCAGCTAAAATA |
| | | TCGCCCAACTGCGGAGCGCCCATCCCAGGGATTGGAGGAGCTGACATAGAGGGTCCTTTA |
| | | CTACTGACGGTACCAGAACTTCCTGAGGCCGATGAAACGACACCACCTCCCACGATTGGG |
| | | GCACTTCTATCATTGGTCTCAGCTTTCTTTAGTTTCATTCCCTTTCTAATGTCACCTAAC |
| | | AGGGCGTCACGGCCCTGCATCACAGATTTTGCCGGTTTAGGAGCACTACCACCTAATGCT |
| | | GGTGGAGGCGGTGGAGGAGGAGGAGCTGGAGCACCTGCCATTTCAACAATTCGTTATATA |
| | | TATGGTAGGCTTCTTCAACAAACGGTTGTGCGCTTTTTAGTCGTATGCTTTGTCTCATAT |
| | | CAAAAACTTTCTTCATAA |
| YPR072W | NOT5 | ATGTCTCAAAGAAAGCTACAACAGGATATCGATAAGCTTTTGAAGAAGGTGAAGGAAGGT |
| | | ATTGAAGACTTCGATGACATATACGAAAAATTTCAGTCAACAGACCCCTCCAATTCTTCG |
| | | CATAGAGAAAAACTGGAATCTGACTTAAAAAGAGAAATCAAGAAGCTGCAAAAACATAGG |
| | | GATCAAATAAAAACATGGCTCAGCAAAGAAGATGTAAAGGATAAGCAAAGTGTTCTGATG |
| | | ACTAATAGGAGGTTGATTGAAAATGGCATGGAAAGATTTAAGTCCGTGGAGAAGCTAATG |
| | | AAGACAAAACAGTTTTCAAAGGAGGCTTTAACAAATCCAGATATAATCAAAGACCCTAAA |
| | | GAACTCAAAAAAAGAGATCAGGTCTTATTTATTCACGATTGTTTAGATGAGCTACAAAAG |
| | | CAATTGGAACAGTATGAGGCTCAAGAAAATGAAGAACAAACAGAAAGACACGAATTTCAC |
| | | ATCGCCAACCTAGAGAATATCTTGAAAAAACTTCAGAATAATGAAATGGACCCAGAGCCC |
| | | GTAGAAGAATTCCAAGACGATATAAAGTACTATGTAGAAAATAACGATGATCCCGATTTC |
| | | ATTGAATACGATACAATTTATGAGGACATGGGTTGCGAAATACAACCTTCATCAAGTAAT |
| | | AATGAGGCTCCAAAAGAAGGAAACAATCAAACTTCTTTATCTAGCATTCGCTCATCAAAA |
| | | AAACAAGAACGTTCTCCAAAGAAAAAAGCTCCGCAGAGGGATGTTTCTATATCAGACAGG |
| | | GCGACGACTCCAATCGCGCCAGGTGTAGAATCAGCCTCGCAATCTATATCTTCTACGCCA |
| | | ACACCTGTGTCAACTGATACGCCATTGCATACAGTGAAAGATGATTCAATAAAATTCGAT |
| | | AATTCTACCCTTGGTACACCAACTACACATGTGTCTATGAAAAAGAAGGAATCGGAAAAC |
| | | GACTCGGAGCAACAACTAAATTTCCCACCGGATAGAACTGATGAAATTCGAAAAACCATC |
| | | CAGCACGATGTAGAGACGAATGCAGCTTTTCAAAATCCCTTATTCAACGACGAATTAAAA |
| | | TACTGGTTAGACTCGAAAAGGTACTTAATGCAACCTCTTCAAGAAATGTCACCAAAGATG |
| | | GTATCGCAATTAGAATCTTCGCTATTGAATTGTCCAGATTCTCTAGATGCCGATTCGCCA |
| | | TGTCTATACACCAAACCATTATCTTTGCCTCACCCAACGTCAATCTTTTTTCCCAATGAA |
| | | CCAATTCGATTTGTTTATCCGTACGATGTACCTTTAAATTTAACGAATAATGAAAATGAT |
| | | ACTGACAATAAGTTCGGTAAAGATAGTAAAGCAAAATCTAAGAAAGATGATGATATCTAT |
| | | TCCAGAACCTCACTAGCCAGAATATTTATGAAATTTGATCTTGATACTTTATTTTTCATA |
| | | TTCTATCATTACCAAGGATCATATGAACAGTTTTTAGCTGCTAGAGAACTTTTTAAAAAT |
| | | AGAAACTGGCTATTCAACAAGGTAGATCGCTGCTGGTATTATAAAGAAATCGAAAAGTTA |
| | | CCACCAGGAATGGGCAAATCTGAGGAGGAATCATGGAGATACTTTGATTATAAAAAAAGT |
| | | TGGTTAGCGAGACGTTGCGGTAACGACTTCGTATATAATGAAGAAGATTTCGAAAAACTG |
| | | TAA |
| YPR173C* | VPS4 | ATGAGCACGGGAGATTTTTTAACTAAGGGAATAGAACTGGTTCAAAAGGCCATTGACCTG |
| | | GACACAGCCACGCAGTATGAGGAGGCGTATACAGCATACTATAACGGATTAGATTATTTG |
| | | ATGTTGGCTTTGAAATACGAGAAAAACCCTAAGTCGAAGGATTTGATAAGAGCGAAGTTT |
| | | ACAGAGTATCTAAACCGTGCAGAGCAGTTGAAAAAGCACTTGGAAAGTGAAGAGGCAAAC |
| | | GCGGCAAAGAAATCTCCTAGCGCTGGTAGTGGTTCCAACGGTGGTAATAAGAAAATCTCT |
| | | CAGGAAGAGGGTGAGGATAATGGTGGAGAAGACAACAAAAAGCTAAGAGGTGCCTTATCG |
| | | AGTGCTATTTTGTCTGAGAAGCCCAATGTTAAGTGGGAAGACGTGGCTGGACTGGAAGGA |
| | | GCCAAAGAGGCCCTTAAAGAAGCCGTTATTTTACCTGTCAAGTTCCCACATTTGTTCAAA |
| | | GGTAACCGTAAGCCCACTTCGGGAATCTTATTGTACGGACCACCAGGTACAGGTAAATCA |
| | | TATTTGGCAAAAGCAGTGGCCACCGAGGCCAATTCTACCTTTTTCAGTGTTAGTTCCAGT |
| | | GATTTGGTTTCTAAGTGGATGGGTGAATCTGAAAAACTTGTCAAACAGTTATTTGCGATG |
| | | GCAAGAGAAAATAAACCTTCTATTATTTTTATAGATGAAGTGGATGCGCTAACAGGTACC |
| | | AGAGGAGAAGGAGAGAGCGAAGCAAGTAGAAGAATTAAAACAGAATTATTGGTACAAATG |
| | | AATGGTGTTGGGAATGACTCCCAAGGTGTTTTGGTCCTTGGCGCAACAAATATCCCATGG |
| | | CAACTGGACAGTGCCATCAGGAGAAGATTTGAAAGAAGAATATATATACCATTACCAGAT |
| | | TTGGCGGCAAGAACCACCATGTTCGAGATTAACGTCGGCGACACTCCATGCGTATTGACC |
| | | AAGGAAGATTATAGAACTTTAGGTGCAATGACTGAAGGTTATTCCGGGAGTGATATTGCT |
| | | GTAGTGGTGAAAGATGCGCTAATGCAACCGATAAGAAAAATTCAAAGCGCGACACATTTC |
| | | AAAGATGTCTCTACGGAGGACGACGAAACAAGAAAACTAACACCATGCTCTCCAGGTGAT |
| | | GATGGCGCCATCGAGATGAGTTGGACGGATATTGAAGCTGATGAATTAAAAGAACCAGAT |
| | | CTCACCATAAAGGATTTCTTAAAGGCTATCAAATCGACGAGACCTACCGTGAATGAGGAT |
| | | GACTTGCTGAAGCAAGAACAGTTCACTAGAGATTTTGGTCAAGAAGGTAACTAG |
| YPL042C* | SSN3 | ATGTATAATGGCAAGGATAGAGCACAAAACTCCTATCAGCCAATGTACCAAAGGCCTATG |
| | | CAGGTACAAGGACAACAGCAAGCTCAATCGTTCGTTGGAAAGAAAAACACAATCGGAAGT |
| | | GTGCATGGAAAAGCCCCGATGCTAATGGCCAATAATGATGTTTTTACTATTGGACCTTAT |
| | | AGGGCAAGAAAAGATAGAATGCGGGTATCTGTCTTAGAAAAGTACGAAGTTATTGGCTAC |
| | | ATTGCTGCGGGCACATATGGTAAAGTTTACAAAGCGAAAAGACAAATCAACTCCGGTACC |
| | | AATTCCGCTAATGGTTCTAGTCTGAATGGTACCAATGCGAAAATTCCGCAGTTTGACAGC |
| | | ACGCAACCAAAATCAAGCTCTTCAATGGACATGCAGGCAAATACAAACGCATTAAGAAGA |
| | | AACTTGTTAAAGGATGAAGGAGTGACCCCCGGAAGAATACGAACTACGAGGGAAGATGTA |
| | | TCCCCGCACTATAATTCCCAAAAACAAACCCTCATTAAAAAACCGCTGACGGTATTTTAT |
| | | GCCATTAAAAAGTTCAAGACAGAGAAGGATGGCGTCGAACAATTGCATTATACGGGAATA |
| | | TCTCAGAGTGCCTGTAGAGAAATGGCATTATGTCGAGAATTGCACAACAAGCATTTAACC |
| | | ACATTAGTGGAAATTTTTTTGGAAAGGAAATGTGTCCATATGGTATACGAATATGCGGAG |
| | | CATGATCTGCTACAAATTATCCACTTCCATTCCCATCCCGAAAAAAGGATGATACCACCA |
| | | AGAATGGTTCGGTCTATTATGTGGCAGCTTTTAGACGGCGTATCGTATCTTCATCAAAAT |
| | | TGGGTGCTTCATCGAGATTTGAAACCCGCAAATATAATGGTGACCATAGATGGATGTGTT |
| | | AAAATTGGTGATTTAGGTTTGGCCAGAAAATTTCATAATATGCTGCAAACCCTCTATACT |
| | | GGGGATAAAGTGGTTGTCACTATATGGTACCGTGCACCTGAGTTGCTATTGGGAGCACGG |
| | | CACTATACCCCTGCGGTTGATTTATGGTCCGTTGGCTGCATTTTTGCAGAACTGATAGGA |
| | | TTACAGCCCATATTTAAAGGTGAAGAAGCTAAACTAGACTCTAAAAAGACTGTTCCATTT |
| | | CAAGTGAATCAACTACAGAGAATTTTGGAAGTTCTTGGCACTCCCGATCAAAAAATTTGG |
| | | CCTTATTTGGAGAAGTATCCAGAATATGATCAAATTACGAAGTTTCCAAAGTATAGGGAT |
| | | AACCTTGCTACATGGTATCATTCCGCGGGAGGAAGGGACAAGCATGCTTTAAGCTTACTT |
| | | TACCACTTGTTAAATTATGATCCAATTAAAAGAATAGATGCATTTAATGCGTTGGAACAT |
| | | AAGTACTTCACAGAAAGTGATATTCCTGTTAGTGAAAATGTATTTGAAGGTCTAACTTAC |
| | | AAATACCCGGCAAGAAGAATTCACACGAACGATAATGACATCATGAATCTTGGATCAAGA |
| | | ACGAAAAACAATACACAAGCTTCAGGAATCACCGCAGGTGCCGCTGCAAATGCGTTAGGT |
| | | GGGCTTGGTGTTAACCGTAGAATTCTGGCCGCGGCAGCAGCAGCCGCTGCTGCGGTGTCA |
| | | GGAAACAATGCATCAGATGAGCCATCTCGAAAGAAAAACAGAAGATAG |
| YPL045W | VPS16 | ATGAAAAACCCTAGCTTCGACTGGGAAAGATTGAAAGACGTATTTTATAGAAGCAGAGCC |
| | | ATTGGCGAATTAAAATGGCCAACTCAGTACGAGGAGTTCAAATGTGCCTTGTCATTAACT |
| | | GTTATCGCGGTTGAAATTCAAGACTTTATACAAGTATACAATTATTTTGGGCAGCTGTTA |
| | | GGCAAGATAAATTTGCAAAGAATTCATGAGGATATAATAAAGTTTGAGTTCGATAAAGAC |
| | | GAGAAGCTGATATTGGTCACGAAAAGCTCCATCAAAATTGTGAAAGGTTGGTCGCCACTG |
| | | ACGATAGAAAGTGTCCCATTACAGGACCCAACCATTGATACTATCTGGGACTATCACAAT |
| | | GGAATCATGTTGTTAGCAAAATCTCGTGATATATACAAGTTAAACGGAAATGAATGGGAA |
| | | CTTCTTTACGAGAACAAAGATAAAAAATATAATTTATTGACAAAAAACCACTGGAGTTGC |
| | | AATGATGACTCCATCATTTTACTGGATGTTGATCATGTATATCAAGTAAGCACCTCGAAC |
| | | GGGGCACTATTGAAGTTAATCACAGATTCGTCGTGGCACAAGGTAACGATCTCATCTAGA |
| | | GGATTTATTTGCCTTTATAATATGAAGGACAATAAACTGCAGATTTTCAGAGATCCAGCA |
| | | AGAATACTAATGGAACACAACTTGGATTCTACACCAGATGATATATGTTGGTGTGGCAAT |
| | | GATACCGTTGCTTGTTCTTTCGAGGACGAAATCAAATTATACGGCCCAGATGGTTTGTAT |
| | | GTAACATTTTGGTACCCTTTTACTGTAACTAACCTAAGGGCAGAGGTTGATGGGTTGAAG |
| | | GTTATTACCACTGAAAAGATTTACTTTCTATCCAGGGTACAACCTCAGACTTCTAATATA |
| | | TTTAGAATTGGCTCAACCGAGCCTGGTGCAATGCTTGTTGACTCATTTAGTTTACTGGAG |
| | | GATCATGCACCCAAAGCAATTGAAATATTAAAGAATTTTGTCTTAGAGAAGGGTGTACTG |
| | | GACTGTATTGCTGCTGCAATAGACGAATTTGAACCTAAACTACAGAAGATGTTGTTGAAC |
| | | GCTGCATCTTATGGAAAAGCTTCTTTGCAGTACAAGTCATTTGACGCTAGTATATTTGTG |
| | | AATGCGTGTAATACGATAAAATTATTAAACTGCTTCAGAAGTTTTGGTATTTTTTTGACT |
| | | GTGGAGGAATACAGGTGTATTTCTTTGAAAGGTGTCATTGATAGGCTGTTGAAGTATCAC |
| | | AGATATTATGAGTGCATACAGATATGCAAATTGGCCAATGAACGATTTCTGTTAGGATAC |
| | | GTGTTTACTGAGTGGGCAAAGGATAAAATCAAGGGTAGTCCTGACATGGAAGATGATGAG |
| | | TTACTAGACAAAATCAAATCTCGACTAAGTGTTATAGATATGACGGATACATTACAAATG |
| | | GTAGCAGTTGCGAAAGTTGCATATCTTGAAGGAAGGTTCCAGTTATCCCGTAATTTAGCT |
| | | TTATTAGAGAAAAACGAAGAAGCTAGAATTGAGCAACTGTATAATCTTGATGATGATAGT |
| | | ATAGCGCTAAAGGAGTGTATCAAAGTACAAAATTACAGTCTGACAATTTCATTACTGATT |
| | | GCACTTTCCAAAAAGCTGACAAACTCTCAACTGACGAAGCTTTTGATCATTGATATGTTC |
| | | AACAATCCCTTATATCTCTATTATATGAGAATGGATAAAGCATACTTGTACGATTTTTAC |
| | | CGCCAAACTGACCGATTTATTGACTTGGCTCACGTATTACTGCAGCAGGGAAAAGAACAG |
| | | CAATCTTTACACAGCTTTTTACCGCAAATTAAGGACCTTTATAGCCAAGTTCAAAACAGT |
| | | GAAGTGGTCAACAATACAATAGAACAACTTCAAAGACAGGAAAAGTTGTGGATATACCAA |
| | | GAATCATTAGGCAAAAGATTTGCTATATCATTCACTAATATGACTTTAGATCAAACGCTT |
| | | TCGAAGTTAATAGAAACAGGGCAAGATAAACAGGTGAAAGAAATAGTTAAAAAGTTCAAA |
| | | ATAAGCGAAAAAAAGCTGTATCACCTGAAATGTAAAACGTTAGTCGAAGCTAAGAAGTTT |
| | | GACGAACTACTACAGTTTGCTCAGTCGCGGAAATCACCAATTGGTTACATGCCTTTCTAC |
| | | ACATACTTAAAAAGCAGAGGCCATATGGATAAAGCTAGTCCTTATGTTAATATGATACCT |
| | | GGCCTTTCGTATCAGGAAAAGAAAAAGCTTTATGTGGAGTGCAGGGGTTTTCGTGATGCA |
| | | ATTCAACTAGCAGGAAAGGAAAAAGACATTCCAGGCTTAAAGGAGATATATAATATTATA |
| | | CCACCTAATGAGCCGGAATTAAAGGCATTAGCAAATGAAACTATGAGCAGGATATAG |
| YKR004C-A* | ECM9 | ATGCAATCTTCTCTTCCCCTTTGCAGAGAATTTTTTGAAAAAATAACTGCATATTTAGAT |
| | | TATCATGATTTCAGGTTAACCATTACTGCCAATCAGCCTTCAATTACTCTCCCTTACTAC |
| | | GTAGACGAAAAAGCCCATTCCATAGAACTAATTATCTTCAAAACAACATTTTTATCTCTT |
| | | TTCCAAGAAGCACATACATATTTTAACAAAACTTTCTCTGATCAAAGCGGCATTTCTAAT |
| | | GAGAATATTTATTACATGACTGTTGGATTTCTACTTACAACACCTGAAAACAAAACGGTA |
| | | TATAATGTACACGAAGATTTACTCAAGAGATACTTCCAGGACAACAGTGTTTTAGTAATA |
| | | CCGGATTTATTGGTCAAAGAAGTAAGACTGATTCAACGGCTTCTTTGTTCATCAAACAAT |
| | | CGTATCAACAAGTCGTCATCTCTTTGGATATTATACCGAAAACTATTCGTTTTATCTCTT |
| | | GATGCTAATACACTGGTTCTTCCAGATATTTTATTTGTATTTCATTCTTCTGGATCCCAA |
| | | CATTTCTCTAATTACTACTGTTGGAATACTGCTAGGTGGTTCTACGATAATTTACCTTAC |
| | | AATAAAAGAATTGAGCTCTTCAATTTGACTAAGAGATTTTGCTTTCAAAATGTTAAGGAT |
| | | TGTTCCTCATGGAGCGCATTAGCATATATGGTATGCCAGCAGGAGGAAAAAAAAACCGAT |
| | | AATATACGTGATTTTCAAAGACTAACAAGCTCTTTTAATGTTCCGTTCAAGATAAATAAA |
| | | GTGGATTTGAATTTTCAAGTACAGCCTGCGGATGCATTTACTCAGGAATTAGTAAAATGG |
| | | ATTGATCGAACGTATGCTGCAGATTGGCCTCCATATCTGTGTCTTCTGCAAATTACGAAA |
| | | TTTAATATCACCCTTAGAATTGAAATGGACTCTGTCTTATTGACTTGGAGAAATGAAATT |
| | | CTGAATTTTGAAGAGAACAGTGGTCATATCAAAATGATAAATAATACTCCAATAGTACCT |
| | | GAGAAATTTTCAAACGATCTTCTAACTTCTGTGAATTTTGCACATTTTGGCTATAAAAAG |
| | | CTTTTCTTAAATAAGTTTTTGGATAAAAATAAGAAAGAACAATCAGATAGTTAG |
| YLR370C* | ARC18 | ATGCCTGCGTATCACTCGACTTTCCCTGTAGATCCTAACACTGACCGCATGGTAGGGAAC |
| | | TTTGCGCTTCTGCCCTTGAACACTAAGTTCAGGGGTCCTGCATACCCCAGCAATTCCGAT |
| | | TACGATATTATCGACGAGTGCTTGGACCTATTTCGTGCCAATTCCTTCTTCAAGAATTTT |
| | | GAAATTAAATCACCCGCCGACAGAGTGCTCATCTACGGAATTCTCTTTATCAATGATTGC |
| | | CTAGCCCATTTGAAAATAACAACGTCATTTAACGAAGCCGTCAAAGTGTTAACCAATGTG |
| | | GCCTTGGACAACTTTACTTTACCAGGTACACCGGGTTTCCCTCTGAATAATGTTTACCAA |
| | | GTTCCAGTACAAGATCATAATTCGATGGACTTGTTGAAGACTTATATTCAGCAATTCCGT |
| | | CAAGAATTGGCAATGAGACTATTAGAAAGAGTCTATAGCTCTACAGATTCTAAGGAATAT |
| | | CCCTCAAAGTTTTGGTTGGCGTTCACAAGAAGAAGATTCATGAACAAATCTCTATAA |
| YLR417W | VPS36 | ATGGAGTACTGGCATTATGTGGAAACTACGTCATCGGGCCAACCTCTTCTTCGAGAAGGT |
| | | GAAAAAGATATCTTTATCGATCAGTCCGTAGGTCTTTACCATGGCAAATCTAAGATTCTA |
| | | CAGCGACAGAGGGGCAGAATCTTTTTAACGTCACAAAGAATTATCTACATCGACGATGCA |
| | | AAGCCCACTCAGAACTCGCTTGGGTTGGAATTGGACGATCTAGCGTATGTAAACTATTCA |
| | | TCTGGCTTCTTGACCAGATCACCTCGTCTGATCCTGTTTTTCAAGGACCCCTCCAGTAAG |
| | | GATGAACTTGGTAAAAGTGCGGAAACAGCAAGTGCCGACGTTGTTTCGACGTGGGTCTGC |
| | | CCCATCTGTATGGTTTCGAACGAAACGCAGGGGGAATTCACTAAAGACACTCTACCTACA |
| | | CCCATTTGTATCAATTGCGGTGTGCCAGCGGACTATGAGCTCACCAAGTCGTCTATAAAT |
| | | TGTTCTAATGCAATCGACCCGAACGCCAACCCACAGAATCAGTTCGGAGTGAACTCTGAG |
| | | AACATTTGCCCCGCCTGTACTTTTGCTAATCATCCTCAAATAGGAAATTGCGAGATTTGC |
| | | GGCCACAGATTGCCTAATGCATCCAAAGTACGATCAAAGCTGAACAGGCTAAACTTTCAT |
| | | GATTCTAGGGTCCATATTGAGCTAGAAAAAAATTCACTGGCTAGAAACAAGAGCTCGCAC |
| | | TCGGCATTATCGTCCTCGTCCTCGACAGGATCATCGACAGAATTCGTGCAATTGAGTTTC |
| | | CGCAAATCTGATGGCGTATTGTTTTCACAAGCTACAGAGAGGGCTTTGGAGAATATACTC |
| | | ACTGAAAAGAATAAGCATATTTTCAATCAGAATGTGGTTTCTGTAAATGGTGTAGACATG |
| | | AGGAAAGGGGCTAGTTCTCACGAATACAACAATGAGGTGCCATTTATTGAGACTAAATTG |
| | | AGCAGAATTGGCATATCAAGCTTGGAAAAATCTAGAGAAAACCAGCTTTTGAACAATGAC |
| | | ATTCTCTTTAACAATGCGTTGACCGACCTAAACAAGCTAATGTCGCTGGCTACCAGCATC |
| | | GAAAGACTATACAAAAATAGCAACATAACGATGAAAACCAAGACATTGAACTTACAGGAT |
| | | GAATCAACCGTGAATGAACCGAAAACGAGAAGACCGCTATTGATACTTGATAGAGAGAAG |
| | | TTCCTAAATAAAGAGCTGTTCTTGGATGAGATTGCGAGAGAAATTTACGAGTTCACGTTG |
| | | TCCGAGTTTAAAGATTTGAACAGTGATACCAACTATATGATCATAACTCTGGTAGATCTA |
| | | TATGCAATGTACAACAAATCTATGCGTATAGGTACAGGCCTGATATCTCCCATGGAAATG |
| | | AGAGAAGCATGCGAAAGATTCGAGCATTTAGGTCTAAATGAATTGAAGTTAGTGAAAGTT |
| | | AACAAAAGAATATTATGTGTAACGAGCGAAAAATTCGACGTTGTAAAAGAAAAGCTAGTA |
| | | GATCTGATCGGTGATAATCCTGGTTCTGATCTCCTAAGATTGACACAGATTTTGAGTTCC |
| | | AATAATTCAAAATCAAACTGGACGCTAGGTATCCTTATGGAGGTATTACAAAATTGTGTC |
| | | GATGAAGGCGATTTACTGATAGACAAGCAACTGAGTGGGATTTACTACTATAAAAACTCT |
| | | TATTGGCCCTCGCATATATAA |
| YML008C* | ERG6 | ATGAGTGAAACAGAATTGAGAAAAAGACAGGCCCAATTCACTAGGGAGTTACATGGTGAT |
| | | GATATTGGTAAAAAGACAGGTTTGAGTGCATTGATGTCGAAGAACAACTCTGCCCAAAAG |
| | | GAAGCCGTTCAGAAGTACTTGAGAAATTGGGATGGTAGAACCGATAAAGATGCCGAAGAA |
| | | CGTCGTCTTGAGGATTATAATGAAGCCACACATTCCTACTATAACGTCGTTACAGATTTC |
| | | TATGAATATGGTTGGGGTTCCTCTTTCCATTTCAGCAGATTTTATAAAGGTGAGAGTTTC |
| | | GCTGCCTCGATAGCAAGACATGAACATTATTTAGCTTACAAGGCTGGTATTCAAAGAGGC |
| | | GATTTAGTTCTCGACGTTGGTTGTGGTGTTGGGGGCCCAGCAAGAGAGATTGCAAGATTT |
| | | ACCGGTTGTAACGTCATCGGTCTAAACAATAACGATTACCAAATTGCCAAGGCAAAATAT |
| | | TACGCTAAAAAATACAATTTGAGTGACCAAATGGACTTTGTAAAGGGTGATTTCATGAAA |
| | | ATGGATTTCGAAGAAAACACTTTCGACAAAGTTTATGCAATTGAGGCCACATGTCACGCT |
| | | CCAAAATTAGAAGGTGTATACAGCGAAATCTACAAGGTTTTGAAACCGGGTGGTACCTTT |
| | | GCTGTTTACGAATGGGTAATGACTGATAAATATGACGAAAACAATCCTGAACATAGAAAG |
| | | ATCGCTTATGAAATTGAACTAGGTGATGGTATCCCAAAGATGTTCCATGTCGACGTGGCT |
| | | AGGAAAGCATTGAAGAACTGTGGTTTCGAAGTCCTCGTTAGCGAAGACCTGGCGGACAAT |
| | | GATGATGAAATCCCTTGGTATTACCCATTAACTGGTGAGTGGAAGTACGTTCAAAACTTA |
| | | GCTAATTTGGCCACATTTTTCAGAACTTCTTACTTGGGTAGACAATTTACTACAGCAATG |
| | | GTTACTGTAATGGAAAAATTAGGTCTAGCCCCAGAAGGTTCCAAGGAAGTTACTGCTGCT |
| | | CTAGAAAATGCTGCGGTTGGTTTAGTTGCCGGTGGTAAGTCCAAGTTATTCACTCCAATG |
| | | ATGCTTTTCGTCGCTAGGAAGCCAGAAAACGCCGAAACCCCCTCCCAAACTTCCCAAGAA |
| | | GCAACTCAATAA |
| YML062C* | MFT1 | ATGCCTCTGTCACAAAAACAAATAGACCAAGTTAGAACCAAAGTGCACTACAGTGAAGTA |
| | | GATACTCCATTCAATAAATATTTAGACATCTTAGGAAAAGTAACCAAGCTGACAGGAAGC |
| | | ATTATAAATGGCACATTATCCAATGATGATAGCAAGATTGAAAAACTTACTGAGCAAAAT |
| | | ATCTCCCAACTAAAAGAAAGTGCTCATCTTCGATTTTTGGATCTGCAGTCATCAATTGAC |
| | | ACAAAGAAAGTAGCAGACGAAAATTGGGAAACATGCCAACAGGAGACATTGGCCAAGCTG |
| | | GAAAATCTCAAAGATAAATTGCCTGATATAAAGAGCATCCATAGCAAGTTGCTTTTACGT |
| | | ATTGGAAAACTACAAGGTCTTTACGATTCTGTCCAAGTAATTAACAGAGAGGTGGAAGGC |
| | | TTGTCAGAAGGCCGTACCAGCCTTGTGGTGACACGTGCGGAATGGGAGAAAGAACTTGGC |
| | | ACTGATCTGGTCAAGTTTTTAATTGAAAAGAATTATCTCAAACTAGTTGATCCGGGCCTG |
| | | AAAAAGGATAGTTCAGAGGAGAGATATCGTATTTATGACGATTTCTCTAAGGGCCCAAAA |
| | | GAGTTAGAAAGTATCAACGCCTCAATGAAATCGGATATAGAAAACGTAAGGCAGGAGGTA |
| | | TCGTCTTACAAAGAGAAGTGGCTAAGAGATGCAGAAATATTTGGCAAGATCACATCAATA |
| | | TTCAAAGAAGAACTTCTGAAGAGAGATGGCCTGCTCAATGAGGCAGAAGGAGATAACATT |
| | | GATGAAGATTATGAATCGGATGAAGATGAGGAAAGAAAAGAGAGGTTTAAAAGGCAGAGA |
| | | TCAATGGTGGAAGTGAATACTATAGAAAATGTGGACGAAAAAGAGGAAAGCGATCATGAA |
| | | TATGACGATCAGGAGGATGAAGAAAATGAAGAGGAAGATGATATGGAAGTAGACGTTGAG |
| | | GATATAAAAGAGGATAATGAAGTTGATGGGGAAAGCAGTCAACAAGAAGATAATAGTCGC |
| | | CAGGGTAATAATGAGGAAACAGACAAAGAAACTGGAGTGATAGAAGAGCCAGATGCGGTT |
| | | AATGACGCAGAGGAGGCAGATAGCGATCACTCAAGCAGGAAACTTGGAGGCACTACAAGC |
| | | GATTTTAGTGCGTCTTCCTCTGTTGAAGAAGTAAAATGA |
| YML094W | GIM5 | ATGTCCTCTCAAAAAATCGATTTAACTAAACTAAATCCCGAGCAGCTGAATGCTGTAAAA |
| | | CAGCAATTCGACCAAGAATTGCAGCATTTCACACAGTCCTTGCAAGCATTAACCATGGCT |
| | | AAGGGCAAGTTCACAGAATGTATTGATGATATTAAAACAGTCTCCCAAGCAGGAAATGAA |
| | | GGGCAAAAACTACTGGTTCCAGCATCTGCATCATTATACATCCCAGGTAAGATTGTAGAC |
| | | AATAAGAAATTCATGGTCGACATTGGTACAGGATATTACGTTGAAAAGAGCGCTGAAGCA |
| | | GCAATCGCATTTTACCAAAAGAAAGTAGACAAGCTTAACAAAGAATCTGTACAGATCCAA |
| | | GATATTATTAAGGAAAAGACCCAGTATTCCTTGTCCATCGAGGCCCAAATAAGACAAGCC |
| | | GCTATTAGACAGCATGAAGCGATGAGTAAACAACAACAGCAGCAACAAAAAAAGGAGTCG |
| | | TCTACAGCCTAG |
| YML112W | CTK3 | ATGGACTCTCTTGAAGCTAGATTACAATTCATTCAGGTCCTGAAGAACCTGCAAAAGACG |
| | | CTGCACAAGACCAGAGACTCTATCACATCATCGTCGACCACCACACCACCGTCATCGCAA |
| | | CAAAAGCTGAACAATGACCCTATACAGTTCTACTTGAGAAACTACAGACATCACTACGAG |
| | | GACTTCCACCAATGTTTGTTCGATACAACCATGAAGATGGACCCACTAGATAGACTGGAC |
| | | GTAGTGATATACTATGTTAGAATAATAAGAAACTTATATCCGCATAGCCATTCCAATACC |
| | | AATGTTACAAAAGTGTTAAACGAAGTGCTACTCATGGACATAGACTTGGTTTTTGAGCTT |
| | | TGTCTGCCCTGCCAGGACTGGAAATCCCTCACGAATCAAGCCACCTGTAAAGAGCTATTC |
| | | CTTGACTTATCCAAACTAATCCATTACGACGCCACCAGCGTCACGCACACACCATCAGAC |
| | | ACCACACTCATAGACGCTACTACCTGGTACAGTGTCAAGACAGAGAGGACTACAAAGGAC |
| | | TATAAAGAATCATTGCAACGAACGGAGTCTCTGCTTAAGGACAGAGACTTGAAGAAATTG |
| | | GCTTTCTTTCAGCAGTTCAATTCCGATACAACTGCGATCAACCCGGACTTACAGACGCAG |
| | | CCAACCAATGCAAACATTTTGTTGCACAGGATGGAAGCAGACAGGGAACTGCACAAGAGG |
| | | TCGAAAGAGACAAGTTGGTACATCGAAAGGCCCTCCAACGATATACTGGATGAATCCGAA |
| | | TTTAAAAGCTTGTGGACGCATTTTGAAACTACTGATTCCGGATTTGATAAGGACGATTAC |
| | | AAAAATATCAAGGCTTTAAATGACATTGCGAAGGCATCTTACATATATTAG |
| YMR032W | HOF1 | ATGAGCTACAGTTATGAAGCTTGTTTTTGGGACCCAAACGACAATGGTGTGAACATCCTT |
| | | CTGGGTCACATTTCTCAGGGAATAAGATCTTGCGACTCAATGATACTTTTCTTTAAGCAG |
| | | CGTAGTGAGCTTGAGAAGGACTATGCCAGGCGGCTTGGAGCCATCACGGGAAAACTAGAC |
| | | AAAGACATTGGAACAAACATGGATTATGGAAAGTTGAATGAAACATTTAATGTGGTGCTC |
| | | AGTGTTGAGAAAGCTCGAGCACAATCGCATTCCAAGCAAAGTGAGATTCTTTTCAGACAG |
| | | ATTTACACGGATACTAAGGCATTTGCCGCTAACTTGCAAGCAAGATATACTACATTGAGT |
| | | GGAAAGATTGAAAGGTTGCGGATGGACAAGTTCAACAAGAAAAAGGGGTGTGAGGTGTTG |
| | | CAAAAGAAATTACAGGATGCCCAGATTAGATTCAGAGACTTGCAATTGAACGAGAATAAT |
| | | ATGATTGGGGCCAAGAGAGTAGAACACAACAAGAGAGAACTATTGAAATGGGAATCGAAC |
| | | TCTCAGGAATATAAGGTTCAACTAGACGTTCTTAAGCAAGAATATAAGGCATCGCAGAAA |
| | | TTCTGGATACACGAGTGGGCGCAATTATCCTGCGAACTGCAAGAAATGGAGAACGCTAGA |
| | | ATATCCTTTTTACAATCGAAATTACAACAGTTCGCAACCTCATCGATGGAAACATATATT |
| | | TTAGAGCAAACAAAAATGGACATGTTAACAAATCATCTGAACTCATTCACGGCAGCGGAT |
| | | GAAATATCGACGTTTTCAAAAGAAAATGGAACTGGTAGACTGAAACATAAGACTTCCAAA |
| | | GGTGACATGAATTCTAGCGCCAACTGGGCTCAAATGAGCAGTATCTCCACGACAAGTAAA |
| | | AAAACCGAATCGTATATGGACAATATAAGGAAACTATCTTCTCAGCTGAAGGAAACCGAA |
| | | AATAAAAGGAAACTAGCTTCAATAGACAAATATGAGAAACCATTGCCATCTCCAGAAGTC |
| | | ACCATGGCTACACAATTTAGAAATTCCACACCCGTCATACGTAATGAGACAAAAGTGGTT |
| | | GCTAACCCAACATTATCTTTGAGGTCTTCACCCGTCCAGTTACAGAGCAATGTGGATGAC |
| | | TCCGTGTTAAGGCAGAAACCTGATAAACCAAGGCCGATTGTTGGGGAAGAACAACTTAAA |
| | | CCTGATGAAGATTCGAAAAATCCTGATGAAAAAGGTCTAATGGTGCATAAAAGAAATCAA |
| | | TCTCTCAGCTCACCATCAGAATCAAGTTCTTCTAATCCAACGGATTTTAGCCACATCAAA |
| | | AAGAGACAAAGTATGGAATCTATGACTACATCCGTAAGTTCAATGGCCAATAGTATAGAC |
| | | GACTCACAGAGATTTGCCAAATCATGGAACTCGTCAAATAGGAAAAGAAAATCTATGAGC |
| | | CATTTGCAAGTACCCTCATCCGCTTCTTCGAGGTCGGATGATGGGGGAAGAACGCCTAAC |
| | | TCTGCGCACAACCTCAATGAAGATGATTATAACACGAGGAGAGATACCAGTACAAGCACA |
| | | ATTTTGTTCAAACCTCCTGTGGCAGTAAGAGGAACGTCTAGGGGGCACACGCATAGACAA |
| | | TCCATGATAATGCAAGATTCAAGTAATCCAATTGAAGATGCCTTGTACGAAATGGAAAGA |
| | | ATCCAAAGTAGTTCTAAACCAGGTACAAAAACAGGAAACATCATGGATGAGAGAGGTGTG |
| | | GTCAGGGATAGAGGTATTACTGTTACCTTGCCTATTGTCACCAGTGAAGGTTTTCCAGTC |
| | | ATTGAATATGCCAAGGCCATGTATCCATTGATTGGAAATGAGGCACCTGGATTAGCGAAT |
| | | TTCCATAAGGGCGACTATCTGCTAATTACTGAGATTGTTAATAAAGATTGGTATAAAGGT |
| | | GAAGTTTATGATAATGACCGTATAGATAGAAATCATAGGATAGGATTAATTCCCTATAAT |
| | | TTCATTCAGCTACTGCATCAAGGTCTTTGA |
| YMR077C* | VPS20 | ATGGGCCAAAAAAGTAGTAAAGTGCATATAACCAAAACAGATAGGGCGATCCTAGAAGTA |
| | | AAACGATCTAAAGATGAAATTCATAAGTTTACGAGGAGGACAGATAATTTGATTTTGGTA |
| | | GAGAAGAGTCAACTAAAAGACCTAATACGAAAAAATCCTGAAAATTATAAATCTAACATG |
| | | AAGGTACGATTTCTACTAAAAAGAATTCATTATCAGGAGCATTTGTTGCAGCAGGCTTCG |
| | | GATCAGCTTATTAATTTGGAAAATATGGTGTCAACACTTGAGTTCAAAATGGTAGAAAAG |
| | | CAATTTATTAATGGACTGAAAAATGGAAATGAAATCTTGAAAAAACTAAACAAGGAATTT |
| | | AGCAATGTTGATGAACTGATGGACGATGTACAAGATCAAATTGCGTATCAAAATGAAATT |
| | | AACGAGACATTATCTAGAAGCTTAGTCGGAACAAGCAATTATGAAGACGATCTAGATAAG |
| | | GAATTGGATGCTCTAGAAAGTGAACTCAATCCAGAGAAGATGAATAACGCAAAAGTAGCT |
| | | AACATGCCATCAACAGAAGGTTTGCCATCATTGCCTCAAGGAGAACAAACCGAGCAAAAA |
| | | GAAAGAGAGGAATTCGCGACGGAGGAGAGATCAGACACTAAGGAACCTTTAGCATTACTA |
| | | TCCTGA |
| YMR202W | ERG2 | ATGAAGTTTTTCCCACTCCTTTTGTTGATTGGTGTTGTAGGCTACATTATGAACGTATTG |
| | | TTCACTACCTGGTTGCCAACCAATTACATGTTCGATCCAAAAACTTTGAACGAAATATGT |
| | | AACTCGGTGATTAGCAAACACAACGCAGCAGAAGGTTTATCCACTGAAGACCTGTTACAG |
| | | GATGTCAGAGACGCACTTGCCTCTCATTACGGGGACGAATACATCAACAGGTACGTCAAA |
| | | GAAGAATGGGTCTTCAACAATGCTGGTGGTGCGATGGGCCAAATGATCATCCTACACGCT |
| | | TCCGTATCCGAGTACTTAATTCTATTCGGAACCGCTGTTGGTACTGAAGGGCACACAGGT |
| | | GTTCACTTTGCTGACGACTATTTTACCATCTTACATGGTACGCAAATCGCAGCATTGCCA |
| | | TATGCCACTGAAGCCGAAGTTTACACTCCTGGTATGACTCATCACTTGAAGAAGGGATAC |
| | | GCCAAGCAATACAGCATGCCAGGTGGTTCCTTTGCCCTTGAATTGGCTCAAGGCTGGATT |
| | | CCATGTATGTTGCCATTCGGGTTTTTGGACACTTTCTCCAGTACTCTTGATTTATACACT |
| | | CTATATAGAACTGTCTACCTGACTGCCAGGGACATGGGTAAGAACTTGTTGCAAAACAAA |
| | | AAGTTCTAA |
| YMR263W | SAP30 | ATGGCTAGGCCAGTTAATACAAACGCTGAAACGGAATCGAGAGGCAGACCCACTCAGGGT |
| | | GGTGGTTACGCAAGTAACAACAATGGCAGCTGTAACAACAACAATGGCAGCAATAATAAC |
| | | AATAATAACAATAATAATAATAATAATAACAGCAATAACAGCAATAACAATAACGGGCCT |
| | | ACCTCCAGCGGGAGAACCAATGGGAAGCAAAGACTCACAGCTGCACAACAGCAGTACATT |
| | | AAGAATCTCATAGAGACACATATCACCGACAACCACCCTGATCTGCGCCCCAAGAGTCAC |
| | | CCAATGGATTTCGAAGAGTATACGGATGCGTTCCTGAGAAGGTATAAAGACCACTTTCAA |
| | | TTGGACGTACCGGACAACCTGACGCTGCAGGGATACTTATTGGGTTCCAAATTGGGCGCG |
| | | AAGACTTATTCATACAAGAGGAACACACAGGGGCAGCACGATAAAAGGATACATAAGAGA |
| | | GACCTGGCCAACGTGGTGAGAAGGCATTTCGATGAGCATTCCATTAAAGAGACAGATTGC |
| | | ATACCGCAATTTATATACAAGGTAAAAAACCAGAAGAAGAAATTCAAGATGGAATTTCGG |
| | | GGTTGA |
| YNL054W | VAC7 | ATGACAGAAGAAGATAGAAAGCTCACTGTAGAGACAGAAACAGTTGAGGCACCCGTGGCA |
| | | AATAATCTTTTATTGTCGAATAACAGTAATGTAGTAGCACCTAATCCTTCTATTCCCTCT |
| | | GCCTCCACATCTACCTCTCCGCTACACAGGGAAATAGTTGATGATTCTGTCGCTACTGCT |
| | | AACACCACCAGCAACGTTGTACAGCATAATTTGCCCACCATAGATAACAATTTAATGGAT |
| | | TCCGATGCCACGTCACATAATCAAGATCATTGGCATTCAGACATAAACAGGGCAGGAACA |
| | | TCAATGTCAACGAGTGATATCCCAACAGATTTACATTTAGAACATATCGGCTCTGTTTCA |
| | | TCAACTAATAATAATAGTAACAATGCCCTAATCAACCACAACCCTCTGTCATCTCATCTT |
| | | TCCAATCCGTCATCTTCATTACGAAACAAGAAGAGCTCTTTGTTGGTAGCTTCTAACCCT |
| | | GCGTTTGCTTCAGATGTTGAACTCTCGAAGAAGAAACCTGCCGTCATCTCCAATAATATG |
| | | CCTACAAGTAACATTGCCCTTTATCAAACAGCGAGATCGGCGAATATTCATGGTCCATCA |
| | | TCAACTTCCGCATCTAAAGCGTTCAGAAAGGCTTCGGCCTTCTCCAATAACACGGCACCC |
| | | AGCACTAGTAATAACATCGGCTCGAATACACCTCCAGCTCCTCTTTTACCTCTACCTTCA |
| | | CTATCACAACAAAATAAGCCAAAAATAATAGAGAGGCCCACAATGCACGTCACTAATTCA |
| | | AGAGAAATACTTTTAGGTGAAAACCTGTTAGATGATACAAAGGCGAAGAATGCTCCCGCG |
| | | AATTCAACCACACACGATAATGGTCCAGTAGCAAATGATGGGCTGCGTATACCGAATCAC |
| | | TCGAACGCAGATGATAATGAAAATAACAACAAAATGAAGAAGAATAAAAACATTAATAGT |
| | | GGGAAAAATGAACGTAATGATGACACCAGCAAAATATGCACTACATCTACTAAAACAGCG |
| | | CCTTCAACCGCACCTTTGGGCAGTACAGACAATACTCAGGCTCTCACTGCTAGTGTCTCC |
| | | AGCAGTAATGCTGACAATCACAATAATAACAAGAAGAAAACCAGCAGCAACAACAACGGC |
| | | AATAACAGTAATAGTGCATCCAATAAAACCAATGCCGATATCAAGAATTCTAACGCCGAC |
| | | TTGAGCGCTTCTACCTCTAACAATAATGCAATAAATGACGACTCACATGAGAGTAATTCA |
| | | GAAAAACCAACAAAGGCGGATTTTTTCGCTGCAAGGCTGGCTACAGCTGTAGGTGAAAAT |
| | | GAAATTAGTGATTCTGAGGAAACATTTGTTTATGAATCGGCAGCTAATTCGACTAAAAAC |
| | | CTAATATTTCCTGACTCCTCCAGCCAGCAGCAGCAGCAGCAACAGCAACCTCCAAAACAA |
| | | CAGCAACAGCAACAAAATCATGGAATAACCTCAAAGATAAGCGCCCCATTGCTAAACAAT |
| | | AACAAAAAATTATTAAGCCGACTGAAAAATTCAAGACATATTAGCACTGGTGCCATATTG |
| | | AATAACACAATCGCGACTATAAGCACAAATCCGAACTTGAATTCTAATGTGATGCAGAAC |
| | | AATAACAATCTGATGTCGGGACACAATCACCTGGACGAGTTGAGCAGTATAAAACAGGAG |
| | | CCACCGCATCAATTGCAGCAGCAGCAACCACCAATGGATGTACAATCGGTAGATTCGTAT |
| | | ACCTCTGACAACCCAGACAGCAATGTTATTGCCAAGTCGCCTGATAAGAGGTCAAGCTTA |
| | | GTATCCCTATCTAAAGTTTCTCCACATTTACTTTCATCCACATCAAGCAACGGTAACACA |
| | | ATATCGTGTCCCAATGTTGCCACAAATTCGCAGGAATTGGAACCAAACAATGATATTTCA |
| | | ACGAAGAAATCTCTTTCCAATTCTACTTTGAGACATTCCTCTGCTAATAGAAATTCTAAT |
| | | TATGGTGACAACAAAAGGCCTCTTAGAACAACAGTGTCAAAGATATTTGATTCAAACCCT |
| | | AATGGAGCTCCTTTACGGAGATACTCTGGGGTACCGGATCACGTTAATCTAGAAGATTAC |
| | | ATCGAACAGCCGCATAATTATCCAACAATGCAAAATAGTGTGAAAAAGGATGAATTTTAT |
| | | AACAGCAGGAACAATAAGTTTCCCCATGGTTTAAACTTTTATGGTGATAACAATGTTATT |
| | | GAAGAGGAAAATAATGGTGACTCGTCTAATGTAAATCGACCGCAACACACTAACCTTCAG |
| | | CATGAGTTTATTCCAGAAGATAACGAAAGTGATGAAAACGATATTCACTCCATGTTTTAT |
| | | TATAATCATAAGAACGATTTAGAAACAAAACCGCTAATATCCGATTATGGTGAAGATGAA |
| | | GACGTAGATGATTATGATCGCCCAAATGCTACTTTCAACAGTTACTATGGCTCAGCATCC |
| | | AACACGCACGAACTTCCATTACATGGAAGGATGCCTTCAAGATCAAATAATGATTACTAC |
| | | GATTTTATGGTTGGCAACAATACTGGCAATAACAACCAATTGAATGAATATACCCCCTTA |
| | | AGAATGAAACGTGGTCAAAGACACCTATCAAGAACAAACAATAGCATAATGAATGGTAGC |
| | | ATCCATATGAATGGTAACGATGACGTTACCCATTCCAATATCAATAATAACGATATTGTT |
| | | GGTTACTCACCGCACAACTTTTACTCAAGGAAGTCCCCATTTGTGAAAGTAAAGAATTTT |
| | | CTTTATCTTGCATTTGTTATATCATCACTATTGATGACAGGATTCATTCTGGGATTTTTA |
| | | TTGGCCACTAATAAAGAACTACAAGATGTAGACGTGGTAGTGATGGATAATGTGATTTCA |
| | | AGTTCGGACGAGTTGATCTTCGACATCACAGTAAGTGCTTTTAATCCAGGATTCTTCAGT |
| | | ATAAGCGTTTCCCAAGTCGATTTGGACATTTTTGCAAAAAGTTCTTACCTGAAGTGCGAT |
| | | TCTAATGGTGACTGTACAGTAATGGAACAGGAACGGAAAATTTTACAAATAACGACAAAT |
| | | CTTTCGTTAGTTGAAGAGAGTGCTAATAATGATATTAGTGGTGGGAACATAGAGACGGTA |
| | | TTACTAGGAACCGCTAAAAAACTAGAGACACCATTAAAGTTCCAGGGCGGCGCATTTAAT |
| | | AGGAACTACGATGTGTCAGTCTCGAGTGTCAAGCTTTTAAGTCCTGGGTCTCGTGAAGCC |
| | | AAGCACGAAAACGACGATGATGACGATGATGATGGCGACGATGGTGACGATGAAAACAAT |
| | | ACTAATGAAAGACAATACAAAAGCAAACCAAATGCTAGAGATGACAAAGAAGATGATACT |
| | | AAAAAATGGAAGCTACTAATCAAGCATGATTACGAATTGATAGTCCGTGGAAGCATGAAG |
| | | TATGAGGTGCCCTTTTTCAATACGCAAAAATCTACGGCTATTCAAAAGGATTCCATGGTC |
| | | CATCCTGGTAAGAAGTGA |
| YNL064C* | YDJ1 | ATGGTTAAAGAAACTAAGTTTTACGATATTCTAGGTGTTCCAGTAACTGCCACTGATGTC |
| | | GAAATTAAGAAAGCTTATAGAAAATGCGCCTTAAAATACCATCCAGATAAGAATCCAAGT |
| | | GAGGAAGCTGCAGAAAAGTTCAAAGAAGCTTCAGCAGCCTATGAAATTTTATCAGATCCT |
| | | GAAAAGAGAGATATATATGACCAATTTGGTGAAGATGGTCTAAGTGGTGCTGGTGGCGCT |
| | | GGCGGATTCCCAGGTGGTGGATTCGGTTTTGGTGACGATATCTTTTCCCAATTCTTTGGT |
| | | GCTGGTGGCGCACAAAGACCAAGAGGTCCCCAAAGAGGTAAAGATATCAAGCATGAAATT |
| | | TCTGCCTCACTTGAAGAATTATATAAGGGTAGGACAGCTAAGTTAGCCCTTAACAAACAG |
| | | ATCCTATGTAAAGAATGTGAAGGTCGTGGTGGTAAGAAAGGCGCCGTCAAGAAGTGTACC |
| | | AGCTGTAATGGTCAAGGTATTAAATTTGTAACAAGACAAATGGGTCCAATGATCCAAAGA |
| | | TTCCAAACAGAGTGTGATGTCTGTCACGGTACTGGTGATATCATTGATCCTAAGGATCGT |
| | | TGTAAATCTTGTAACGGTAAGAAAGTTGAAAACGAAAGGAAGATCCTAGAAGTCCATGTC |
| | | GAACCAGGTATGAAAGATGGTCAAAGAATCGTTTTCAAAGGTGAAGCTGACCAAGCCCCA |
| | | GATGTCATTCCAGGTGATGTTGTCTTCATAGTTTCTGAGAGACCACACAAGAGCTTCAAG |
| | | AGAGATGGTGATGATTTAGTATATGAGGCTGAAATTGATCTATTGACTGCTATCGCTGGT |
| | | GGTGAATTTGCATTGGAACATGTTTCTGGTGATTGGTTAAAGGTCGGTATTGTTCCAGGT |
| | | GAAGTTATTGCCCCAGGTATGCGTAAGGTCATCGAAGGTAAAGGTATGCCAATTCCAAAA |
| | | TACGGTGGCTATGGTAATTTAATCATCAAATTTACTATCAAGTTCCCAGAAAACCATTTC |
| | | ACATCAGAAGAAAACTTGAAGAAGTTAGAAGAAATTTTGCCTCCAAGAATTGTCCCAGCC |
| | | ATTCCAAAGAAAGCTACTGTGGACGAATGTGTACTCGCAGACTTTGACCCAGCCAAATAC |
| | | AACAGAACACGGGCCTCCAGGGGTGGTGCAAACTATGATTCCGATGAAGAAGAACAAGGT |
| | | GGCGAAGGTGTTCAATGTGCATCTCAATGA |
| YNL084C* | END3 | ATGCCCAAGTTGGAACAATTTGAAATAAAAAAATACTGGCAAATCTTCTCGGGTTTGAAA |
| | | CCAATAGAGAATAAGGTAAACCATGACCAGGTTTTACCAATTCTTTATAATTCCAAATTA |
| | | GATTCATCGGTTCTAAACAAGATTTGGTTTTTAGCTGATATTGATGACGACGACAATTTA |
| | | GACTTTGAGGAATTTGTAATTTGCATGAGATTAATATTTGATATGGTTAATAAAAACATT |
| | | AGCTCTGTTCCAGATGAATTGCCTGATTGGTTGATTCCAGGGAGTAAGGTGAATCTAATC |
| | | AAAGAAAGAAAGAAGCGCAAGCAAATAGAAAACGCAGACTTGCCTCCAAAGAAGGAAATC |
| | | AAAGTAGATTGGTACATGTCTCCAGATGATTTGAATCAATATGAAAAAATTTATAATAGC |
| | | TGTGCAAAACTAACCGATGGTACTATTACATTCAACGAACTGTCAACAAAACTTTCCACA |
| | | AAATTTTTTAACATCAGTAAGACAGATTTAAATAAGGTTTGGAGTTTAATTAACCCACAA |
| | | AATTTGCCATCAATTGATAGAGATCCTACTTTTTATTTTATTCACTGTTTAAGACAAAGA |
| | | AATGATTTGGGTGCTGAAATTCCAGCAAGTTTACCAAATTCTCTGGCGGAGGTGTGCAAT |
| | | AAAAAACAACTGAGCTATGATTTACGGTCCTCTCAACCTCCTACAAAGAGAAAAGAAGAA |
| | | GCTAACGAAGTGGACAATCTGCGTGACAATGGGCAGAACTCGAGTTCCGATAGCAGTGGC |
| | | AGTAACGTGCTTTCAAATGAAGACAGTATAAAGCAAAAATATGCTTCTCTGACAGATGAT |
| | | CAAGTTGCGAACATGAGAGAACAATTAGAAGGCCTTTTGAACTACAAAAAGAGTGAAAAA |
| | | ACTCAAGGAGGCTCAAAACTTTCTAAGCGGATTAACATCAGGTCGATAACTGATGACTTG |
| | | GATAACATTGAACAGCAGGTTGAAGTATTGGAGAATTACTTGAACAATAAGAGACACGAA |
| | | TTGCAAGCATTACAAGCAGAAATCAATTGA |
| YNL097C* | PHO23 | ATGAGTTCACCAGCGAACCTATTCCCTGGATTAAACGACATAACTGACGTGCTGGAGGAA |
| | | TTCCCACTGGCCACCTCCAGATATTTAACATTACTACACGAAATAGATGCAAAATGTGTG |
| | | CATTCTATGCCGAATTTGAACGAGAGGATAGATAAGTTCTTGAAGAAAGACTTCAATAAA |
| | | GATCACCAAACACAAGTAAGACTGCTCAATAATATCAACAAGATTTATGAAGAACTGATG |
| | | CCATCGCTGGAGGAGAAAATGCATGTCTCATCCATTATGCTGGATAATCTAGACAGATTG |
| | | ACGTCCCGGTTAGAATTGGCGTATGAAGTCGCAATCAAGAACACAGAAATTCCTAGAGGT |
| | | TTAAGACTGGGTGTGGACAACCATCCAGCAATGCACCTCCATCATGAACTAATGGAAAAA |
| | | ATAGAGAGCAAATCAAACAGCAAATCGTCGCAGGCACTGAAGAGCGAATCAAGAAGAGAA |
| | | GCCATGGCTGCCAACAGGAGGCAGGGCGAACATTACTCCGCCAGCACACACCAACAAGAC |
| | | GACTCAAAGAACGACGCAAACTACGGAGGCAGCAGGCACGAGAGCCAAGACCACACTGGT |
| | | AACAACACAAACTCAAGAAAAAGAGCCAACGCTGCCAATACCAACAACGCCGATCCAGAG |
| | | ACCAAAAAACGCAAGAGGAGAGTTGCCACCACAGCCGTTTCACCAAGCACTATCAGCACG |
| | | GCAACTGCCGTCAATAATGGCAGGATAGGTACATCTACAGCGTCCAGGGGAGTTAGCAGC |
| | | GTCGGAAACAGCAACAACAGCAGGATATCAAGACCAAAAACCAACGACTACGGCGAACCG |
| | | CTCTACTGCTACTGTAACCAAGTGGCATACGGGGAAATGGTGGGGTGTGATGGCGCAGAC |
| | | TGTGAGCTAGAATGGTTCCATTTGCCATGTATTGGACTCGAAACTCTACCTAAGGGCAAG |
| | | TGGTATTGCGACGACTGCAAAAAAAAACTGTGA |
| YNL133C* | FYV6 | ATGTCATCTACCAGTGATAATAATGCTAATAGTGCAAGAGAGAAAAAACCACTGAAATTC |
| | | GTGTCAGAAGGAGTTGGTAATGTAGAGGCCCAGAGGATACGAGAGCAAGTGGAGCAAAAA |
| | | AAATATGAAGCGGAATATAAAAGGAAAACCAGGAAGTCCCTGCGAGATCAGCTTAGGTCT |
| | | AATGCTATTAGTAAACAAAAACAATATAACGGTTTGGTGAGAGACCGTGAAAGTTTTACT |
| | | AGGTTGAGTAAAGAGGACCTGGAATTTTACCAGAAGAGCAAGAATGAGTTGCTAAAAAAG |
| | | GAAAAAGAACTGAATAACTACTTAGACGTCAAGGCAATCAATTTTGAAAAGAAAAAAAAA |
| | | GCATTACTAATGGAGAACGATTCTACTACTAATACAGAAAAATATTTAGAGACCGGCACT |
| | | AGTTTGGGCAGTAAAACACAAATAAAGGGCGTAAAGACATCCAGCCCTAAACCAAAAATC |
| | | AAGGTATCCATAAAAAAATTGGGTAGAAAACTGGAGAACTGA |
| YNL140C* | | ATGCTGGAGGCAAAGATGGTTATGGTAGTTCCAACTATAGAGGACCAAGCAATGATCGTT |
| | | CTGCACCCAAAAATATTAAGCCAATCAGTAGTTATGCACATAAAAGGTCAGAACTACCAA |
| | | CTAGACCTAGTAAAAGTAAAACATATAATGATAGAAGTAGGGCACTTAGACCCACAGGTC |
| | | CGGACAGGGGCGATGGTTTTGATCAAAGGGATAACCGCTTACGTGAAGAGTATAAGAAAA |
| | | ACAGTTCACAACGTTCACAATTACGGTTTCCTGAAAAGCCATTTCAAGAGGGCAAAGACA |
| | | GCAGCAAAGCCAACCCTTACCAAGCTTCATCTTACAAGCGCGACTCGCCTTCAGAAAATG |
| | | AAGAAAAGCCGAATAAAAGATTCAAGAAGGATGAAACAATTAGAAACAAATTTCAAACGC |
| | | AAGATTATAGAAATACACGAGATAGCGGTGCTGCCCATAGAGCCAATGAAAATCAAAGGT |
| | | ACAACGGAAATAGAAAGAGCAATACTCAGGCGCTTCCGCAAGGTCCCAAGGGTGGGAATT |
| | | ACGTCAGTAGGTACCAGAGGTAATTACTAG |
| YNL147W | LSM7 | ATGCATCAGCAACACTCCAAATCAGAGAACAAACCACAACAGCAAAGGAAAAAATTCGAA |
| | | GGCCCTAAAAGAGAAGCTATTCTGGATTTAGCGAAGTATAAAGATTCTAAAATTCGCGTC |
| | | AAATTAATGGGTGGTAAATTAGTTATAGGTGTCCTAAAAGGCTATGATCAACTGATGAAC |
| | | TTGGTACTTGATGATACAGTAGAATATATGTCTAATCCTGATGATGAAAACAACACTGAA |
| | | CTGATTTCTAAAAACGCAAGAAAGCTAGGTTTGACCGTCATAAGAGGTACTATTTTGGTC |
| | | TCTTTAAGTTCCGCCGAAGGTTCTGATGTACTATATATGCAAAAATAG |
| YNL229C* | URE2 | ATGATGAATAACAACGGCAACCAAGTGTCGAATCTCTCCAATGCGCTCCGTCAAGTAAAC |
| | | ATAGGAAACAGGAACAGTAATACAACCACCGATCAAAGTAATATAAATTTTGAATTTTCA |
| | | ACAGGTGTAAATAATAATAATAATAACAATAGCAGTAGTAATAACAATAATGTTCAAAAC |
| | | AATAACAGCGGCCGCAATGGTAGCCAAAATAATGATAACGAGAATAATATCAAGAATACC |
| | | TTAGAACAACATCGACAACAACAACAGGCATTTTCGGATATGAGTCACGTGGAGTATTCC |
| | | AGAATTACAAAATTTTTTCAAGAACAACCACTGGAGGGATATACCCTTTTCTCTCACAGG |
| | | TCTGCGCCTAATGGATTCAAAGTTGCTATAGTACTAAGTGAACTTGGATTTCATTATAAC |
| | | ACAATCTTCCTAGATTTCAATCTTGGCGAACATAGGGCCCCCGAATTTGTGTCTGTGAAC |
| | | CCTAATGCAAGAGTTCCAGCTTTAATCGATCATGGTATGGACAACTTGTCTATTTGGGAA |
| | | TCAGGGGCGATTTTATTACATTTGGTAAATAAATATTACAAAGAGACTGGTAATCCATTA |
| | | CTCTGGTCCGATGATTTAGCTGACCAATCACAAATCAACGCATGGTTGTTCTTCCAAACG |
| | | TCAGGGCATGCGCCAATGATTGGACAAGCTTTACATTTCAGATACTTCCATTCACAAAAG |
| | | ATAGCAAGTGCTGTAGAAAGATATACGGATGAGGTTAGAAGAGTTTACGGTGTAGTGGAG |
| | | ATGGCCTTGGCTGAACGTAGAGAAGCGCTGGTGATGGAATTAGACACGGAAAATGCGGCT |
| | | GCATACTCAGCTGGTACAACACCAATGTCACAAAGTCGTTTCTTTGATTATCCCGTATGG |
| | | CTTGTAGGAGATAAATTAACTATAGCAGATTTGGCCTTTGTCCCATGGAATAATGTCGTG |
| | | GATAGAATTGGCATTAATATCAAAATTGAATTTCCAGAAGTTTACAAATGGACGAAGCAT |
| | | ATGATGAGAAGACCCGCGGTCATCAAGGCATTGCGTGGTGAATGA |
| YNL236W | SIN4 | ATGATGCTTGGAGAGCATTTAATGAGCTGGTCTAAGACTGGTATTATTGCATACAGTGAC |
| | | TCACAGTCCTCAAATGCCAATATTTGCCTCACATTTTTAGAATCCATAAATGGAATAAAC |
| | | TGGCGATTTCATACGCCGCAAAAATATGTATTACACCCGCAATTGCACGAAGTTCAATAC |
| | | CAAGAAAGTAGTAGTACTCTAAGCACTCACTCGACAACAACTTCTGTCAATGGATCAACT |
| | | ACAGCCGGGGTTGGAAGCACTCCAAACTTCGGAGGCAACAGCAACAAGTCTCCTCCACAG |
| | | TTCTTTTATAATATATCTTCCATTCATTGGAACAACTGGTTTAGTTTACCAGGTGACATG |
| | | TTGGCTGTATGTGACGAATTAGGTAACATGACCATGTTAATTACTGGACAACGTCCTGAT |
| | | CGGGCCACTACATACGAAAAGTTAACGATGGTATTTCAAGATAATGTTTACAAAATTTAT |
| | | AACCACGTTATGCCTTTAAAGCCTGTAGACAAGCTAAAGCCCATGAACATTGAAAGGAAA |
| | | CAAACAAGGAAAGAATACAATACATCCATCTTGGAATTTCGCTGGCTCACCTCTTCAAAA |
| | | TCCGTAATCGTTTCTCAATTCTGCGCATTTGATAGTAGTTCTAATACATATAGAAGCAGA |
| | | GCACAGCAAGTGCCTCCATATGGTGTGTACCATCCACCCTTTATAAAATATGCGTGCTTG |
| | | GCAATAAGGAAAAACGGTCAAATAGATTTTTGGTACCAATTTTCAAACTCAAAGGATCAT |
| | | AAAAAAATAACGTTACAGCTGCTGGACACTTCAAATCAAAGATTCAAGGATCTTCAATGG |
| | | CTTGAATTTGCTAGAATTACACCTATGAATGATGATCAGTGTATGTTGATAACCACATAT |
| | | TCAAAATTATCTAAAAATATCTCTTTTTACAAACTACACGTTAATTGGAACCTTAATGCG |
| | | ACCAAACCAAACGTCCTAAATGACCCATCGTTGAAAATACAATTTATATTAAGCACTACT |
| | | TTAGATCCAACGGATGATGAAGGGCATGTGTTGAAGTTAGAGAACCTGCATGTTGTGTCT |
| | | AAATCATCAATTGAGAAAGATCCCTCCCCAGAAATTTTAGTTTTGTATAATGTTTGCGAT |
| | | ACATCAAAATCATTGGTGAAAAGATATAGACTAGCTCCAACACAACTTTCAGCTGAGTAT |
| | | TTGGTGATATTGAAACCAGATTTAAATATCGATCGAAATAATTCTACGAATCAAATATTC |
| | | CAATCGCGCCGTTACAACCTTCGTCGCCACAGTGATATCGTTCTCGACAAAAAAGTCACC |
| | | TTGATAACTTCTGAGATGTTCGATGCATTTGTTTCATTTTACTTTGAAGATGGTACCATC |
| | | GAATCCTACAATCAAAATGATTGGAAGTTGGAGACTGAACGACTTATAAGTCAGAGCCAA |
| | | TTAGGAAAATTCAAAAATATTATTGCATCCCCACTAAGCGCAGGATTTAATTATGGGAAG |
| | | CTCCCTTTACCTCCATCGGTTGAGTGGATGAAGGTTTCACCATCTATGTGTGGGGTTATC |
| | | GTTAAACAGTATAATAAAAAATGGCCACAGTTTTATGCTGCTGTTCAAAAAAACTACGCA |
| | | GATCCTGAGAAGGATTCTATAAATGCTACAGCTTTGGCATTTGGATACGTGAAAAGTTTA |
| | | CATAAGCAAATATCTGCCGAAGACTTGACCATCGCTGCGAAGACGCACATACTCAGAATT |
| | | TCATTCTTGGATAGAAAAAGGGCCAAAGAATTTATTACGACTTTGTTGAAAAGCCTATAC |
| | | TCGTTTTTCAACATTTCTCCTGATGCGCCTAAAGAGATTATGGATAAAATAATAACAAGT |
| | | AGGCCACTTCAAAAAATTATGCTATTGCAGCTGGAACTTGGCAGTTGTTTTTCGCAAGAA |
| | | AATATTGAAGAAATGGCGAGAGTTATACTCTACTTGAAAAATGTTTTGTTTGCGTTCAAT |
| | | GGTGTGGCGAGAAATTTTCATTTTGCGATTGAACAAATAAGTAATAATTCTAACCAACAA |
| | | CAAAACCCGAAGTTGTTCCAGACTATATTTTCAAAACAGGATCTCATTCATTCATTGATT |
| | | CCTGTTGCTAAATGGTTTGTCAAATTTATCACTTATTTGACCCAAGAAATTTTAATTTTA |
| | | ATAAATGATCCCACAAACAAGGAATACACACTAGTTCACGGAATATTTGGAGCCAAAATG |
| | | TCCAGGACTTTGATATTGTCTATTCTTAACGAAATCAAGAAAGTCACCCAAATAGTGGCG |
| | | AAATTTCCAGAAACATCATACCCAATTTTAAACGAGTCATCAACTTTTTTGAAACTAGTC |
| | | TTATCTGAATCACCAGTGGATTTCGAAAAATTTGAAACGTTTTTAGTTGATGTAAACAAC |
| | | AAATTTATCGCGCTATGCGAACAACAGCCATCACAGGAACGTGAGTTTTCGTTGTTGGTG |
| | | AAAGCTGAAATCCCCCCAGAATATGCCAAAGTGGGAGATTTCCTCCTGCAGTATGCTAAT |
| | | AATGCAGTGATTTCCCATGCGAATGCGGCTGCAGTATATTTTGCAGATACAAGTGGCTTG |
| | | AAAATATCTAATTCAGAGTTTTTCAATCCCGAAATATTTCATTTATTGCAACCATTAGAA |
| | | GAGGGATTAATTATTGATACAGATAAACTACCCATAAAAAATAGAACCTCCAAATCTTTT |
| | | AGCAAATTACTGTACGACGATGTAACCTGTGACAAACTGAGTGTATCAGAAATATCCGAT |
| | | GGGAAACTTAAGAGATGCAGTAGGTGCGGTTCCGTCACAAGAGCAGGAAATATAATATCG |
| | | AGTGATAAGACAATTGTACCCACTAGCATCCAAACCAAAAGATGGCCAACCATGTATACA |
| | | AGACTGTGCATATGTTCAGGTATGCTTTTTGAGATGGACGGCTGA |
| YNR051C* | BRE5 | ATGGGTGTTACCGTTCAAGACATATGTTTTGCCTTTTTGCAAAACTACTACGAAAGAATG |
| | | AGGACCGACCCTTCCAAATTGGCATATTTTTATGCAAGTACAGCCGAGTTGACGCATACT |
| | | AATTATCAGTCCAAGTCAACAAACGAAAAAGATGACGTTCTTCCAACGGTGAAAGTTACT |
| | | GGACGTGAAAACATTAATAAGTTTTTTTCTAGAAATGATGCCAAAGTTAGGAGCTTGAAA |
| | | CTAAAATTAGATACTATAGATTTCCAGTACACTGGGCACTTGCACAAAAGCATTCTGATC |
| | | ATGGCCACGGGTGAAATGTTTTGGACAGGAACTCCGGTCTATAAGTTTTGTCAAACATTT |
| | | ATCCTGCTACCATCCAGCAATGGGTCTACGTTTGACATTACCAATGATATAATCAGATTT |
| | | ATTTCCAATTCATTCAAGCCTTATGTCTTAACAGATGCTTCTTTGTCACAATCTAACGAG |
| | | GAGAACTCTGTCAGTGCAGTTGAAGAAGATAAAATAAGGCATGAGTCCGGAGTTGAAAAA |
| | | GAAAAAGAAAAAGAAAAATCACCAGAAATATCGAAACCAAAGGCCAAAAAGGAAACGGTC |
| | | AAAGACACAACTGCGCCAACTGAATCGTCAACTCAGGAAAAACCAATTGTCGATCATAGT |
| | | CAACCTCGGGCTATACCCGTCACTAAAGAGTCCAAGATTCACACCGAAACGGTACCATCT |
| | | AGTACTAAGGGCAACCACAAACAAGATGAGGTCTCTACAGAAGAGTTAGGAAACGTTACT |
| | | AAACTAAATGAAAAATCTCACAAAGCCGAAAAGAAAGCTGCACCAATCAAGACAAAGGAA |
| | | GGCTCTGTTGAGGCCATAAATGCTGTTAATAACTCTTCTTTGCCCAACGGTAAAGAAGTT |
| | | AGTGATGAAAAACCGGTACCTGGGGGCGTAAAGGAAGCTGAAACTGAAATTAAACCAATA |
| | | GAGCCACAGGTTTCCGACGCCAAAGAATCCGGCAATAACGCTTCAACACCTTCATCTTCT |
| | | CCAGAGCCCGTTGCGAATCCACCAAAGATGACATGGGCCTCAAAGTTGATGAATGAAAAC |
| | | TCCGACAGAATTAGCAAAAATAATACCACGGTTGAATACATCAGACCTGAAACACTACCC |
| | | AAGAAACCGACTGAAAGAAAATTTGAAATGGGGAATCGGAGGGACAACGCTAGTGCTAAC |
| | | AGCAAAAATAAGAAAAAACCAGTATTCAGCACCGTTAACAAGGACGGCTTTTATCCAATC |
| | | TACATTAGAGGTACAAATGGCTTAAGAGAAGAAAAATTGAGAAGTGCCTTAGAAAAAGAA |
| | | TTCGGTAAAGTTATGAGGATAACAGCTGCAGACAATTTTGCCGTCGTCGACTTTGAAACT |
| | | CAAAAGAGCCAAATCGATGCTTTAGAGAAGAAGAAAAAGTCAATTGATGGTATTGAAGTA |
| | | TGCTTAGAAAGAAAAACAGTAAAGAAGCCCACGAGCAATAATCCTCCTGGCATATTTACT |
| | | AATGGAACACGTTCTCATAGAAAGCAACCCCTAAAAAGAAAGGACTAG |
| YNR052C* | POP2 | ATGCAATCTATGAATGTACAACCGAGGGTATTAGCTGTAGGAGGTGAGCAGTTTTTCTCT |
| | | CAGAGGCAGGCTTCTGAACAACACCAGCAGCAAAACATGGGACCTCAGGTGTATTCGCCA |
| | | AAGGTTAATAGAGCAAGAATGTTTCCTCAAGGGATGCCTGTAAATACAATTAATGGCTCT |
| | | GTTAATCAGGAGATGAACAATGCCTATCTTTTGAAGCAGAAGAACGAGCCACTGTTAACT |
| | | CAGCAACAACAGCAACAGCAACAACAACAGCAGCCATTTAATATAGGAACGCCTGTTTCG |
| | | GTGGCTAGTTTGCCGCCCGGACTGAACGTTCTGCAGCAACAACAACAGCAGCAACAACAA |
| | | CAGCAGCAACAGCAGCAGGGAGTGGGACTCAATCGTCCCTTGGCTTCCCAGCTACCCAAG |
| | | CATTTAACCAACCAAAGTATGCCACCGATCTTTTTACCACCCCCAAATTATCTTTTCGTT |
| | | CGCGATGTCTGGAAGTCCAACCTTTACAGTGAATTCGCCGTTATAAGACAACTGGTCTCG |
| | | CAGTATAATCATGTGTCAATCAGTACTGAGTTTGTGGGGACTTTGGCTAGGCCGATCGGC |
| | | ACTTTCAGGTCGAAGGTCGATTACCACTATCAGACAATGAGGGCCAACGTAGACTTCTTA |
| | | AACCCAATACAACTTGGTCTTTCCTTGAGTGACGCCAACGGCAATAAGCCTGACAACGGT |
| | | CCCTCAACGTGGCAGTTCAATTTTGAATTTGACCCAAAGAAGGAAATCATGTCCACAGAA |
| | | TCGTTAGAATTACTCAGAAAATCAGGTATCAACTTCGAAAAACACGAAAATTTAGGCATC |
| | | GATGTATTTGAATTTTCGCAGCTTCTAATGGACTCTGGTTTAATGATGGATGATTCTGTT |
| | | ACTTGGATCACATACCATGCGGCATATGATCTAGGTTTCCTGATCAACATTCTAATGAAT |
| | | GACTCCATGCCCAACAACAAGGAGGATTTCGAGTGGTGGGTCCATCAATACATGCCCAAT |
| | | TTCTACGACTTAAACCTGGTGTATAAAATAATCCAGGAGTTCAAGAACCCTCAATTGCAA |
| | | CAATCTTCACAACAGCAACAGCAGCAGCAATATTCACTAACCACACTTGCCGATGAGTTA |
| | | GGGCTACCAAGATTCTCTATATTTACTACTACAGGCGGCCAGAGTCTATTGATGCTTCTA |
| | | TCCTTCTGCCAATTGAGCAAGCTGTCTATGCATAAGTTCCCAAACGGTACGGATTTCGCC |
| | | AAGTACCAAGGTGTCATATACGGTATTGATGGGGACCAATGA |
| YOL004W | SIN3 | ATGTCACAGGTTTGGCATAATTCGAATTCGCAATCAAACGATGTGGCTACTTCAAATGAC |
| | | GCTACGGGTTCCAACGAAAGAAACGAAAAAGAACCGTCCCTCCAGGGAAATAAGCCCGGT |
| | | TTTGTTCAACAGCAGCAACGGATTACTTTACCCTCGCTATCTGCCTTGAGTACTAAGGAG |
| | | GAAGATAGAAGAGATTCCAATGGCCAACAGGCTCTAACTTCTCATGCTGCTCACATATTA |
| | | GGTTATCCTCCCCCACATTCAAATGCTATGCCCTCAATTGCAACTGATTCAGCATTGAAA |
| | | CAGCCCCACGAGTATCACCCTCGCCCTAAATCTTCGTCCTCTTCTCCCTCTATAAACGCT |
| | | TCGCTTATGAATGCTGGTCCAGCTCCCCTCCCCACAGTGGGAGCCGCCAGTTTTTCTTTG |
| | | TCGAGATTTGACAATCCATTACCGATAAAAGCTCCTGTTCATACAGAGGAACCAAAAAGT |
| | | TATAATGGTCTTCAGGAAGAAGAAAAGGCGACGCAACGGCCTCAAGATTGCAAGGAAGTT |
| | | CCCGCTGGTGTGCAGCCTGCTGATGCCCCTGACCCTAGCAGCAACCATGCAGATGCTAAC |
| | | GATGACAATAATAACAACGAAAATTCTCACGATGAAGATGCTGACTACAGACCTCTAAAC |
| | | GTGAAGGATGCCCTATCTTACCTCGAACAGGTCAAATTTCAATTTAGTTCGCGTCCGGAT |
| | | ATCTATAATTTATTTTTAGATATTATGAAGGACTTTAAATCTCAGGCAATAGACACACCG |
| | | GGCGTTATTGAAAGAGTATCCACTTTGTTCAGAGGTTATCCAATTTTGATTCAAGGGTTC |
| | | AATACTTTTCTACCCCAAGGCTATAGAATCGAATGCTCCTCTAATCCGGACGACCCCATT |
| | | AGAGTTACTACACCAATGGGTACTACGACAGTAAACAATAACATCAGTCCATCTGGTAGA |
| | | GGTACAACGGATGCACAGGAACTTGGTTCTTTTCCAGAAAGCGATGGAAATGGTGTTCAA |
| | | CAGCCCTCCAATGTGCCAATGGTGCCTTCGAGTGTGTATCAATCGGAACAAAACCAAGAC |
| | | CAACAACAATCTTTGCCTCTTTTAGCTACTTCTTCTGGTTTACCTTCAATTCAACAACCT |
| | | GAAATGCCTGCACATCGCCAAATCCCACAAAGTCAATCTTTAGTGCCTCAAGAAGATGCT |
| | | AAGAAAAACGTTGATGTCGAATTTAGTCAAGCCATAAGCTACGTTAATAAAATTAAAACT |
| | | AGATTTGCCGACCAACCTGATATTTACAAGCATTTTCTGGAAATACTACAAACTTATCAG |
| | | CGAGAGCAAAAGCCAATAAACGAAGTCTACGCACAAGTGACGCATCTTTTCCAAAATGCT |
| | | CCTGATTTACTAGAAGATTTCAAGAAATTCTTGCCGGACTCTTCAGCTTCTGCCAATCAG |
| | | CAGGTGCAACATGCTCAGCAACATGCTCAACAACAACATGAGGCCCAAATGCATGCACAG |
| | | GCACAAGCTCAGGCTCAGGCACAGGCACAGGTGGAACAACAGAAGCAGCAACAGCAATTC |
| | | TTGTATCCAGCTTCGGGCTATTACGGCCATCCTTCTAACCGAGGTATTCCACAGCAAAAC |
| | | TTGCCTCCTATAGGAAGCTTCTCGCCTCCAACAAACGGTTCGACCGTACATGAAGCCTAT |
| | | CAGGATCAACAGCACATGCAACCACCCCACTTCATGCCCTTACCATCGATCGTTCAACAC |
| | | GGGCCAAATATGGTACATCAAGGGATAGCGAATGAAAATCCACCTCTATCAGATCTAAGA |
| | | ACGTCTCTCACTGAACAGTACGCTCCTTCTAGTATACAACATCAGCAGCAGCACCCGCAA |
| | | AGTATTAGTCCTATAGCAAATACGCAGTATGGTGATATCCCTGTTAGACCGGAAATTGAT |
| | | TTAGATCCTAGCATTGTGCCTGTGGTCCCTGAACCCACTGAGCCCATCGAAAACAATATA |
| | | TCGCTTAATGAGGAAGTCACTTTCTTCGAAAAGGCCAAGAGGTATATCGGCAATAAACAT |
| | | TTATACACTGAGTTTTTGAAAATTTTAAATTTGTACTCTCAAGATATACTTGATCTTGAC |
| | | GATTTAGTGGAAAAGGTAGATTTCTACTTGGGTTCCAATAAAGAACTATTTACGTGGTTC |
| | | AAAAACTTTGTTGGCTACCAAGAAAAAACCAAATGTATCGAGAATATTGTTCATGAAAAA |
| | | CATAGATTGGATTTAGATTTATGTGAGGCATTTGGCCCAAGTTACAAGAGGCTACCAAAA |
| | | AGTGACACTTTCATGCCATGCTCAGGTAGGGATGATATGTGTTGGGAAGTCTTGAACGAT |
| | | GAATGGGTTGGACATCCTGTATGGGCTTCCGAAGATTCGGGATTTATTGCTCATCGTAAA |
| | | AACCAGTATGAGGAAACACTATTCAAGATCGAAGAGGAAAGACATGAGTATGATTTTTAC |
| | | ATTGAATCAAATTTAAGAACTATTCAATGTTTGGAAACAATTGTAAATAAGATCGAGAAC |
| | | ATGACTGAGAATGAAAAAGCCAATTTTAAACTGCCTCCAGGTCTTGGCCATACTTCAATG |
| | | ACTATTTATAAAAAAGTGATAAGGAAAGTTTATGATAAGGAAAGGGGGTTCGAGATTATT |
| | | GATGCTTTGCATGAGCACCCTGCAGTGACAGCCCCAGTCGTTCTGAAAAGGTTAAAGCAA |
| | | AAAGACGAAGAATGGAGAAGAGCTCAACGTGAATGGAATAAAGTTTGGAGGGAGTTAGAA |
| | | CAGAAGGTTTTTTTCAAGTCATTAGATCATTTAGGCTTAACATTTAAACAGGCTGACAAG |
| | | AAATTATTAACTACAAAGCAGTTGATATCAGAGATTAGCAGCATCAAAGTTGATCAAACA |
| | | AACAAAAAAATTCACTGGTTAACTCCTAAACCAAAGAGCCAGTTAGATTTTGATTTCCCT |
| | | GATAAAAACATTTTCTATGATATCTTGTGTTTGGCTGACACTTTTATAACCCATACCACA |
| | | GCCTATTCTAATCCCGATAAAGAAAGATTGAAAGATTTACTAAAATACTTCATCTCTTTG |
| | | TTTTTTTCTATTTCTTTCGAAAAAATCGAAGAATCGTTGTACTCCCATAAGCAAAACGTG |
| | | TCAGAATCTAGCGGATCTGACGATGGCAGTTCTATTGCATCAAGGAAGAGGCCCTATCAA |
| | | CAAGAAATGAGTTTACTTGATATTTTACACAGGAGCAGATATCAAAAGCTAAAGCGTTCT |
| | | AATGATGAAGATGGCAAAGTTCCCCAGCTCTCTGAACCACCCGAAGAAGAACCTAATACC |
| | | ATTGAGGAGGAAGAGCTCATCGATGAAGAAGCTAAAAATCCGTGGCTAACTGGGAATTTA |
| | | GTGGAGGAAGCAAACTCTCAGGGTATTATCCAAAATCGCAGTATTTTTAATCTATTCGCT |
| | | AATACGAATATTTACATATTTTTCCGTCATTGGACAACGATTTATGAGCGGCTTTTGGAA |
| | | ATTAAGCAAATGAATGAAAGGGTCACAAAGGAAATCAACACAAGGTCGACAGTTACTTTT |
| | | GCCAAAGATCTAGATTTATTATCGAGTCAACTTTCCGAAATGGGACTAGATTTTGTTGGT |
| | | GAAGACGCGTACAAACAGGTTTTAAGACTGAGTAGAAGGTTGATTAATGGGGATCTTGAA |
| | | CATCAGTGGTTTGAAGAGAGTTTGCGGCAAGCTTACAATAACAAAGCGTTTAAACTCTAT |
| | | ACAATTGATAAAGTCACCCAATCGTTGGTAAAGCATGCTCATACCTTGATGACTGACGCT |
| | | AAAACTGCGGAAATAATGGCTTTGTTCGTTAAAGATAGAAATGCCTCCACCACGAGTGCG |
| | | AAGGACCAAATTATCTATCGCTTGCAGGTGCGCTCACATATGTCCAACACAGAAAATATG |
| | | TTTAGAATAGAGTTTGATAAAAGAACTCTGCATGTTTCCATTCAATATATTGCACTTGAT |
| | | GATTTGACACTAAAGGAACCAAAGGCAGACGAAGATAAATGGAAATATTATGTAACATCG |
| | | TATGCTCTGCCACATCCAACAGAAGGCATTCCGCACGAGAAACTGAAGATACCATTTTTG |
| | | GAAAGGCTCATCGAATTTGGACAAGATATTGATGGAACAGAGGTAGATGAGGAGTTTTCC |
| | | CCCGAAGGCATCTCTGTATCGACATTGAAAATTAAGATCCAGCCTATAACCTACCAACTG |
| | | CATATTGAAAACGGGTCTTACGATGTTTTTACCCGTAAGGCTACTAACAAATATCCTACT |
| | | ATTGCTAATGATAATACTCAAAAAGGAATGGTTTCTCAGAAGAAGGAGTTGATATCAAAA |
| | | TTTCTAGACTGTGCAGTTGGTTTGAGAAATAATTTAGATGAGGCTCAAAAATTAAGTATG |
| | | CAAAAAAAATGGGAGAACCTAAAAGACAGCATAGCAAAGACGAGTGCTGGAAACCAGGGA |
| | | ATAGAGAGTGAAACCGAAAAAGGTAAAATTACGAAACAGGAGCAGTCGGATAATTTGGAC |
| | | AGCTCTACTGCAAGTGTACTGCCTGCATCCATTACTACTGTACCACAAGATGATAATATA |
| | | GAAACGACTGGGAATACTGAATCTTCAGACAAGGGGGCTAAGATTCAATAA |
| YOL051W | GAL11 | ATGTCTGCTGCTCCTGTCCAAGACAAAGACACTCTGTCCAATGCCGAGCGTGCGAAGAAC |
| | | GTCAACGGGTTGCTTCAGGTGCTCATGGACATTAACACTCTGAACGGAGGGAGCTCCGAC |
| | | ACTGCTGATAAGATAAGAATTCATGCCAAAAACTTCGAGGCAGCTTTGTTCGCAAAGAGC |
| | | TCTTCAAAGAAAGAATACATGGACAGCATGAACGAAAAAGTTGCTGTCATGCGCAACACG |
| | | TACAATACGAGGAAAAACGCCGTTACTGCTGCTGCCGCTAATAACAACATTAAACCCGTG |
| | | GAACAGCACCATATTAACAACTTGAAAAATTCTGGCAACAGCGCCAATAATATGAATGTC |
| | | AATATGAATCTAAACCCACAGATGTTCTTGAATCAGCAGGCTCAGGCAAGGCAACAGGTT |
| | | GCGCAACAATTAAGAAATCAACAACAACAACAACAACAGCAGCAGCAGCAACAGAGGCGT |
| | | CAATTGACTCCTCAACAACAACAATTAGTGAACCAGATGAAAGTGGCACCTATTCCCAAA |
| | | CAATTACTGCAAAGAATTCCTAACATTCCACCCAATATCAACACCTGGCAGCAGGTCACT |
| | | GCTTTGGCTCAACAAAAGCTATTGACACCTCAGGATATGGAAGCTGCGAAGGAAGTCTAC |
| | | AAGATTCACCAGCAGTTGCTATTCAAAGCAAGGCTACAGCAACAACAAGCACAGGCTCAA |
| | | GCACAAGCTAATAACAACAACAACGGCCTCCCCCAAAATGGTAATATTAACAATAACATA |
| | | AATATTCCTCAACAGCAGCAAATGCAACCTCCCAATTCAAGTGCGAACAACAACCCTTTG |
| | | CAACAGCAATCATCACAAAATACCGTACCAAACGTCCTCAACCAAATTAACCAAATCTTT |
| | | TCTCCAGAGGAGCAACGCAGCTTATTACAAGAAGCCATCGAAACCTGCAAGAATTTTGAA |
| | | AAAACACAATTGGGTAGTACGATGACGGAACCTGTCAAGCAAAGTTTTATTAGGAAATAC |
| | | ATTAACCAAAAGGCCCTGAGAAAAATCCAAGCTTTGAGAGATGTTAAGAACAACAATAAC |
| | | GCTAACAACAACGGCTCGAACCTTCAGAGAGCCCAAAATGTCCCTATGAATATCATCCAA |
| | | CAACAACAACAACAGAACACGAACAATAATGACACCATTGCCACTTCTGCTACACCTAAT |
| | | GCTGCCGCTTTCTCTCAGCAACAGAACGCAAGTTCTAAATTATATCAGATGCAACAACAG |
| | | CAACAAGCTCAAGCTCAAGCTCAAGCTCAAGCTCAGGCACAGGCTCAAGCACAAGCTCAA |
| | | GCACAGGCGGCACAAGCGGCGCAAGCGCAAGCACAAGCACAAGCACAAGCACAAGCACAG |
| | | GCACAGGCACAGGCACAAGCCCAGGCGCAGGCCCAAGCCCAAGCCCAAGCACAAGCACAA |
| | | GCACACGCTCAGCACCAGCCCTCCCAACAACCACAACAAGCTCAGCAACAACCTAACCCA |
| | | CTACATGGGTTGACACCTACTGCAAAGGATGTCGAAGTAATTAAGCAATTGTCCTTGGAT |
| | | GCTTCTAAGACCAACCTAAGGCTTACGGACGTAACAAATTCTTTATCCAATGAAGAAAAG |
| | | GAAAAAATTAAAATGAAGTTAAAGCAAGGTCAAAAGCTTTTTGTTCAGGTGAGTAATTTC |
| | | GCCCCACAAGTCTACATCATCACAAAGAATGAAAACTTCTTGAAGGAAGTTTTTCAGTTA |
| | | AGAATCTTTGTAAAAGAGATCCTAGAAAAATGTGCCGAGGGTATATTTGTTGTTAAATTA |
| | | GACACCGTTGACAGGTTAATTATTAAGTATCAAAAATATTGGGAAAGTATGAGAATTCAA |
| | | ATTTTAAGAAGACAAGCTATTTTAAGACAACAACAGCAAATGGCAAACAACAATGGGAAC |
| | | CCAGGCACTACTTCTACTGGAAACAATAATAATATTGCAACTCAGCAAAATATGCAACAG |
| | | TCACTACAGCAAATGCAGCATTTACAGCAATTGAAAATGCAGCAGCAACAACAACAGCAG |
| | | CAACAACAACAGCAGCAACAACAACAGCAGCAACAACAACAGCAACAGCACATATATCCC |
| | | TCCTCGACTCCTGGTGTGGCTAATTATTCGGCAATGGCTAATGCACCCGGTAACAATATC |
| | | CCATATATGAACCATAAAAATACCTCTAGCATGGATTTTTTGAACTCTATGGAAAATACA |
| | | CCAAAAGTTCCCGTATCCGCTGCGGCAACCCCATCACTGAACAAGACGATCAACGGTAAG |
| | | GTGAATGGCAGGACAAAATCTAATACGATACCTGTTACCAGCATTCCATCAACAAATAAG |
| | | AAACTATCAATTTCAAATGCCGCTAGTCAACAACCCACTCCTAGGTCTGCATCAAATACC |
| | | GCTAAGTCAACCCCAAATACAAATCCTTCTCCACTGAAAACCCAAACTAAAAATGGCACA |
| | | CCAAACCCCAATAATATGAAGACTGTACAGTCTCCTATGGGTGCACAACCATCATATAAT |
| | | AGTGCCATTATAGAGAATGCATTCAGAAAGGAAGAACTCTTGTTAAAAGATTTGGAAATC |
| | | AGGAAGTTGGAGATATCTTCTCGTTTTAAACATCGCCAAGAAATTTTCAAAGATTCTCCT |
| | | ATGGATTTGTTTATGAGTACGCTGGGTGATTGCTTAGGTATCAAAGATGAAGAGATGCTT |
| | | ACGTCATGCACTATCCCAAAGGCTGTGGTTGATCACATCAACGGCTCTGGCAAAAGGAAG |
| | | CCTACAAAAGCGGCTCAAAGGGCTCGCGATCAAGATTCCATTGACATTTCCATAAAAGAC |
| | | AACAAATTGGTTATGAAGAGTAAATTCAATAAGAGCAATAGGTCGTATTCGATAGCGTTG |
| | | TCCAATGTAGCTGCTATATTCAAGGGTATCGGTGGTAACTTTAAAGACTTGTCCACTTTG |
| | | GTTCATTCATCATCGCCGTCCACATCTTCTAATATGGATGTCGGCAACCCAAGAAAAAGA |
| | | AAAGCCAGCGTATTAGAAATAAGCCCGCAAGATTCGATAGCCTCGGTGCTATCACCAGAT |
| | | TCAAATATAATGAGTGATTCTAAAAAAATTAAAGTAGATTCTCCTGATGACCCATTCATG |
| | | ACAAAATCAGGAGCCACAACTAGTGAAAAACAAGAAGTAACAAATGAAGCTCCATTTTTA |
| | | ACTTCTGGGACTAGTTCAGAACAATTCAATGTATGGGATTGGAATAATTGGACAAGTGCT |
| | | ACTTGA |
| YOL068C* | HST1 | ATGAACATATTGCTAATGCAACGGATAGTTAGTTTTATTTTGGTGGTTAGTCAAGGGCGA |
| | | TACTTTCATGTTGGAGAGCTGACCATGACAATGTTGAAAAGACCTCAAGAAGAAGAGTCA |
| | | GATAATAATGCTACGAAGAAGCTGAAAACGAGATTAACGTACCCTTGTATCCTCGGGAAG |
| | | GATAAGGTGACGGGCAAATTCATCTTTCCTGCAATCACTAAAGATGATGTTATGAATGCA |
| | | AGACTGTTTTTGAAGGACAACGATTTGAAAACTTTTTTAGAATATTTTTTGCCAGTGGAA |
| | | GTCAACTCGATCTACATTTATTTCATGATAAAATTGCTGGGATTCGACGTCAAAGATAAA |
| | | GAACTATTTATGGCGTTAAACTCTAATATCACTTCTAATAAAGAACGTTCTAGTGCGGAA |
| | | CTATCGTCTATACATGCAAAAGCTGAAGATGAGGACGAGTTGACCGATCCCTTAGAGAAA |
| | | AAGCATGCAGTAAAATTAATCAAAGATTTACAAAAAGCCATAAATAAAGTCCTTAGTACA |
| | | AGGCTTCGATTACCCAACTTCAATACGATTGATCATTTCACCGCCACTTTGCGTAACGCC |
| | | AAAAAAATTTTGGTTCTGACGGGCGCTGGAGTTTCCACTTCTTTAGGTATTCCCGATTTT |
| | | AGATCATCTGAGGGGTTTTATTCTAAGATCCGACACCTTGGATTGGAAGACCCTCAGGAT |
| | | GTTTTCAACTTGGATATATTCTTACAGGACCCTTCTGTTTTTTACAATATTGCCCATATG |
| | | GTTTTACCACCAGAGAACATGTATTCTCCGTTGCACAGCTTCATTAAAATGCTACAAGAC |
| | | AAGGGCAAACTATTAAGAAACTACACACAGAATATAGATAACTTAGAATCGTATGCAGGG |
| | | ATAGATCCTGATAAATTAGTGCAATGTCACGGTTCATTCGCCACTGCATCTTGTGTGACT |
| | | TGCCATTGGCAAATACCTGGTGAGAAAATATTCGAAAATATTAGAAACCTTGAACTACCA |
| | | TTATGTCCCTACTGTTATCAAAAAAGAAAACAATATTTTCCTATGAGTAATGGCAATAAT |
| | | ACGGTGCAAACAAACATAAATTTCAACTCACCCATTTTAAAGTCGTATGGTGTTTTGAAA |
| | | CCAGACATGACATTTTTTGGCGAGGCCCTGCCATCGCGATTTCATAAGACCATCCGCAAA |
| | | GATATACTGGAATGCGATCTATTGATTTGTATAGGAACAAGTTTGAAAGTGGCTCCTGTG |
| | | TCTGAAATTGTCAATATGGTACCTTCACATGTTCCGCAGATTTTGATCAATAGGGATATG |
| | | GTTACACATGCAGAGTTTGACTTAAACCTGTTGGGATTCTGTGATGACGTGGCCAGTCTC |
| | | GTAGCTAAAAAATGCCATTGGGATATACCGCATAAAAAATGGCAAGATCTAAAGAAGATA |
| | | GATTACAACTGTACGGAAATAGATAAAGGTACGTATAAGATTAAGAAACAGCCACGAAAG |
| | | AAACAACAGTAA |
| YOL086C* | ADH1 | ATGTCTATCCCAGAAACTCAAAAAGGTGTTATCTTCTACGAATCCCACGGTAAGTTGGAA |
| | | TACAAAGATATTCCAGTTCCAAAGCCAAAGGCCAACGAATTGTTGATCAACGTTAAATAC |
| | | TCTGGTGTCTGTCACACTGACTTGCACGCTTGGCACGGTGACTGGCCATTGCCAGTTAAG |
| | | CTACCATTAGTCGGTGGTCACGAAGGTGCCGGTGTCGTTGTCGGCATGGGTGAAAACGTT |
| | | AAGGGCTGGAAGATCGGTGACTACGCCGGTATCAAATGGTTGAACGGTTCTTGTATGGCC |
| | | TGTGAATACTGTGAATTGGGTAACGAATCCAACTGTCCTCACGCTGACTTGTCTGGTTAC |
| | | ACCCACGACGGTTCTTTCCAACAATACGCTACCGCTGACGCTGTTCAAGCCGCTCACATT |
| | | CCTCAAGGTACCGACTTGGCCCAAGTCGCCCCCATCTTGTGTGCTGGTATCACCGTCTAC |
| | | AAGGCTTTGAAGTCTGCTAACTTGATGGCCGGTCACTGGGTTGCTATCTCCGGTGCTGCT |
| | | GGTGGTCTAGGTTCTTTGGCTGTTCAATACGCCAAGGCTATGGGTTACAGAGTCTTGGGT |
| | | ATTGACGGTGGTGAAGGTAAGGAAGAATTATTCAGATCCATCGGTGGTGAAGTCTTCATT |
| | | GACTTCACTAAGGAAAAGGACATTGTCGGTGCTGTTCTAAAGGCCACTGACGGTGGTGCT |
| | | CACGGTGTCATCAACGTTTCCGTTTCCGAAGCCGCTATTGAAGCTTCTACCAGATACGTT |
| | | AGAGCTAACGGTACCACCGTTTTGGTCGGTATGCCAGCTGGTGCCAAGTGTTGTTCTGAT |
| | | GTCTTCAACCAAGTCGTCAAGTCCATCTCTATTGTTGGTTCTTACGTCGGTAACAGAGCT |
| | | GACACCAGAGAAGCTTTGGACTTCTTCGCCAGAGGTTTGGTCAAGTCTCCAATCAAGGTT |
| | | GTCGGCTTGTCTACCTTGCCAGAAATTTACGAAAAGATGGAAAAGGGTCAAATCGTTGGT |
| | | AGATACGTTGTTGACACTTCTAAATAA |
| YOL148C* | SPT20 | ATGAGTGCCAATAGCCCGACAGGAAACGATCCCCATGTATTTGGTATTCCTGTGAACGCA |
| | | ACACCATCCAATATGGGTTCGCCAGGCAGTCCAGTTAATGTACCACCTCCTATGAACCCA |
| | | GCGGTAGCAAATGTAAATCATCCTGTTATGAGGACAAACAGTAATAGTAATGCCAATGAA |
| | | GGTACTAGGACTTTAACCAGGGAGCAAATACAGCAATTGCAGCAAAGACAACGATTATTA |
| | | CTACAGCAAAGACTACTCGAACAACAGAGAAAACAGCAAGCACTCCAAAACTATGAGGCT |
| | | CAGTTTTATCAAATGCTTATGACCTTAAACAAAAGACCTAAAAGACTTTACAATTTTGTG |
| | | GAAGATGCAGACTCAATTTTGAAAAAATATGAGCAATATTTACACAGTTTTGAATTTCAT |
| | | ATTTATGAGAATAATTATAAGATTTGCGCTCCTGCAAATAGCAGGTTACAACAGCAGCAA |
| | | AAGCAACCCGAGCTGACTAGTGACGGTTTAATATTAACCAAAAATAACGAAACTTTGAAA |
| | | GAATTTTTAGAGTACGTCGCAAGAGGAAGGATTCCTGATGCTATTATGGAAGTCTTAAGG |
| | | GACTGTAATATCCAATTTTACGAAGGAAATCTTATTTTACAGGTTTATGACCATACTAAT |
| | | ACAGTTGATGTCACCCCCAAAGAGAACAAGCCTAATTTGAACAGCTCTTCTTCCCCTTCA |
| | | AATAATAATAGCACACAGGACAATTCCAAGATTCAACAACCATCCGAACCTAATAGTGGT |
| | | GTGGCAAATACAGGCGCAAACACAGCAAATAAGAAGGCATCCTTCAAACGACCTAGAGTA |
| | | TATCGAACATTGTTGAAGCCTAATGATTTGACGACGTATTATGACATGATGTCTTATGCA |
| | | GATAATGCCAGGTTTTCTGATAGCATTTATCAACAGTTTGAATCTGAAATTTTAACACTT |
| | | ACCAAGAGGAACTTGTCACTAAGTGTGCCATTAAATCCATATGAGCACCGAGACATGTTG |
| | | GAGGAAACAGCTTTTTCAGAACCTCATTGGGATAGTGAAAAAAAATCCTTCATTCACGAA |
| | | CATCGTGCTGAGTCTACGAGAGAAGGCACGAAGGGCGTTGTAGGGCATATCGAAGAACGC |
| | | GATGAATTTCCGCAACATAGTTCAAATTATGAACAGTTGATGTTGATCATGAATGAACGC |
| | | ACAACAACGATAACCAACTCCACGTTTGCTGTTTCGTTAACCAAGAATGCGATGGAGATT |
| | | GCTAGCTCCAGTTCCAACGGGGTACGTGGAGCTTCGTCCTCAACTTCGAATTCCGCATCA |
| | | AATACAAGGAATAACAGTTTGGCTAATGGCAATCAAGTAGCTTTGGCAGCGGCAGCGGCT |
| | | GCGGCTGCAGTGGGATCTACCATGGGTAACGATAATAATCAATTTAGCAGGCTGAAATTT |
| | | ATTGAACAATGGAGAATAAATAAAGAAAAAAGAAAACAGCAGGCTTTAAGTGCAAACATC |
| | | AATCCAACCCCCTTCAACGCTAGAATATCGATGACAGCACCATTGACTCCACAGCAACAG |
| | | CTACTTCAAAGACAACAGCAGGCCTTGGAGCAACAACAGAATGGTGGAGCCATGAAAAAC |
| | | GCAAATAAACGGAGTGGTAACAATGCCACCAGTAACAATAATAATAATAATAATAATTTA |
| | | GATAAACCAAAGGTTAAGCGACCTAGAAAAAATGCAAAAAAAAGCGAGAGCGGTACACCA |
| | | GCTCCTAAAAAGAAAAGAATGACTAAGAAGAAACAGAGCGCAAGTAGCACGCCCTCTTCT |
| | | ACTACAATGTCATAA |
| YOR026W | BUB3 | ATGCAGATAGTACAAATTGAGCAGGCCCCAAAAGACTACATAAGCGACATCAAAATAATC |
| | | CCTTCCAAGTCACTGCTTTTGATTACGTCTTGGGATGGCTCTTTAACAGTCTACAAATTC |
| | | GACATTCAAGCAAAGAATGTTGACCTTTTACAATCGCTACGATATAAACATCCGTTATTG |
| | | TGCTGCAATTTCATCGACAATACCGATCTGCAAATATACGTGGGAACTGTACAGGGTGAA |
| | | ATTCTAAAAGTTGATTTGATAGGTAGTCCCAGCTTCCAAGCTTTGACGAACAATGAAGCC |
| | | AATTTGGGTATTTGCCGAATATGCAAATATGGAGACGATAAACTCATTGCCGCGTCATGG |
| | | GATGGCCTGATAGAGGTTATCGACCCTCGCAATTATGGTGATGGAGTTATTGCTGTTAAA |
| | | AATTTGAACTCTAATAACACAAAGGTGAAGAATAAGATATTTACTATGGATACAAACTCC |
| | | TCTCGATTGATCGTTGGTATGAACAATAGTCAGGTTCAATGGTTTCGCCTGCCACTCTGT |
| | | GAGGATGATAACGGAACAATTGAAGAATCAGGACTGAAGTACCAAATAAGAGATGTCGCT |
| | | CTTTTACCGAAAGAACAAGAAGGTTATGCATGTAGCAGCATTGACGGGCGAGTTGCTGTG |
| | | GAGTTTTTCGATGATCAGGGCGATGATTACAACTCAAGCAAAAGATTTGCATTTAGATGC |
| | | CACCGTTTGAATTTAAAAGATACAAACTTAGCGTATCCAGTAAATTCTATTGAATTTTCC |
| | | CCCCGTCATAAGTTCCTATACACGGCTGGCTCTGATGGCATAATTTCATGCTGGAACCTA |
| | | CAAACCCGCAAGAAAATAAAAAATTTCGCCAAATTTAACGAAGACAGCGTGGTTAAAATT |
| | | GCTTGTTCGGACAATATTCTATGTCTGGCAACTTCTGATGATACTTTCAAGACAAACGCC |
| | | GCAATTGACCAAACTATTGAACTAAACGCAAGTTCAATATACATAATATTTGACTATGAG |
| | | AACTGA |
| YOR035C* | SHE4 | ATGCCACTGTGTGAGAAAGGGAATGATCCAATCGATAGCTCTACTATTGATAGCCTCTGC |
| | | GCTGCATTTGACAAGACTTTGAAATCCACCCCTGATGTCCAAAAATACAATGATGCTATA |
| | | AATACAATTTTCCAGCTAAGGCAAAAATCTGAATCTGGGAAAATGCCGGCTGATTTAACG |
| | | AATAGTGAAGCCCTTAAAGATCGTCAGAAAATCGAAGAAATATTGACCAGGTCTTACCAA |
| | | GACCATTCTGAGTCCCGCGTACATTTATCTAAACTTATACAGAATGATATTCCCTTCGCA |
| | | CTCAATTTATTTGAAATATTATCAAGATCATCCATCCACGTTTTCGTTGGTTGCTTTTCC |
| | | AATAAAGACGCAACAATTGCATTGTTAAATGAGCTGCAAATTAGGATACACTATGGCGAG |
| | | GACACCCACGTCACGTATTTACTGAGCATAATTCTACAACTATTGAATAAGTTCAAGTAC |
| | | AATTTCAAGGAGGTGCGATTTTTAGTGAAGGAACTTATTTTGCGCATAAGCGAAGATGAA |
| | | GTAAAATCGATGATGCTTATAATTTTTGCTGAATTACAATCAAGCTTCCAAAAAGACTTT |
| | | GATAAAGCAGTGGTCGATTTCATGAGTAGCTTAATTGTGGAGGCAGAAATAGACGTTGGA |
| | | AATGATCCATTGTCCATCATAGTCAAAACCCTATCAGAATTATATCCTTCATTGACAACA |
| | | CTTTGTTCTGAAATATTTTTGACCAAAGGACTAAGTAAGCTTTTTAAGAAGAGAGTATTC |
| | | GAAGAGCAGGATTTACAGTTTACAAAAGAACTGCTGCGGTTATTATCTTCCGCGTGTATT |
| | | GATGAGACCATGAGAACGTATATTACAGAAAATTACCTTCAACTATTGGAAAGATCACTA |
| | | AACGTGGAAGATGTACAAATATATTCTGCATTGGTGTTAGTTAAGACATGGTCTTTCACA |
| | | AAATTAACGTGTATAAACTTGAAGCAACTGTCCGAAATCTTTATAAACGCTATTTCAAGA |
| | | CGTATCATGCCAAAAATTGAAAATGTCAATGAAAGTGCGGTTAAGCTCGAGGAGGTTCCT |
| | | AAAGTTGAAATGTCCGTTGAAGCGCTGGCATATTTAAGTCTAAAGGCCTCGGTTAAGATT |
| | | ATGATTAGGAGTAACGAGAGCTTTACTGAAATTCTATTAACTATGATTAAAAGCCAGAAA |
| | | ATGACGCACTGTTTATATGGTCTCTTAGTTATCATGGCAAATTTGTCCACTTTACCAGAG |
| | | GAATCCAATGGTAGTTCACAGTCTATCAATGACTTAAAAAATTACGCAGATTTGAAGGGC |
| | | CCAGGCGCAGACAAGGTAGGCGCAGAGAAAGAATCAAAGGAAGATATTCTTTTGTTTAAC |
| | | GAAAAGTACATATTAAGGACCGAGCTGATTTCCTTTTTGAAGAGGGAAATGCACAATTTG |
| | | AGCCCCAATTGTAAACAACAAGTGGTTAGGGTTATATATAACATAACCCGCTCGAAAAAC |
| | | TTCATTCCTCAATGCATTTCACAAGGTGGTACAACTATCATCTTAGAATATTTGGCCAAT |
| | | AAGCAGGACATAGGGGAGCCAATACGGATTCTAGGGTGTCGGGCATTAACAAGAATGCTA |
| | | ATATTCACTAATCCGGGTTTGATATTTAAAAAATACTCTGCATTAAACGCTATCCCCTTC |
| | | TTGTTCGAATTGTTGCCAAGGTCTACGCCAGTGGATGACAATCCGTTACATAACGACGAA |
| | | CAAATAAAGCTGACAGACAATTACGAGGCACTATTAGCTCTGACGAACTTGGCATCATCG |
| | | GAGACCTCTGATGGTGAAGAAGTTTGCAAGCATATCGTATCTACAAAAGTATATTGGTCC |
| | | ACGATTGAGAATCTGATGCTAGACGAAAACGTTCCTTTGCAAAGATCAACATTAGAGCTC |
| | | ATAAGTAATATGATGAGTCATCCATTAACGATTGCTGCTAAGTTCTTTAACTTGGAAAAT |
| | | CCTCAGAGTTTGAGAAATTTTAATATATTGGTGAAACTTCTTCAGTTATCCGACGTTGAG |
| | | TCTCAACGAGCAGTGGCTGCTATCTTTGCTAACATTGCCACCACGATTCCATTGATAGCT |
| | | AAGGAACTGTTAACCAAGAAAGAACTAATCGAAAATGCTATTCAGGTATTTGCGGATCAA |
| | | ATTGATGACATTGAGTTAAGACAAAGGTTACTTATGCTTTTTTTTGGATTATTCGAAGTG |
| | | ATACCGGATAATGGCACTAACGAGGTGTATCCGTTGCTGCAAGAAAATCAAAAATTAAAG |
| | | GATGCTTTAAATATGTCTTTAAAGAGAGGCGATTCAGGACCAGAGTTTTCAGCGGCAATT |
| | | CCCGTTATCCTTGCTAAAATTAAAGTCTAA |
| YOR036W | PEP12 | ATGTCGGAAGACGAATTTTTTGGTGGTGATAATGAAGCCGTTTGGAACGGTTCCAGATTC |
| | | AGTGATTCACCTGAGTTCCAAACGTTGAAAGAAGAGGTTGCTGCAGAGTTATTTGAAATA |
| | | AATGGGCAAATAAGCACGCTGCAGCAGTTTACCGCGACACTTAAGTCATTTATAGATCGG |
| | | GGAGATGTTAGTGCGAAAGTTGTGGAAAGAATTAATAAGAGATCTGTGGCAAAGATAGAA |
| | | GAAATAGGCGGGCTCATTAAAAAGGTCAATACATCAGTGAAAAAGATGGATGCGATTGAG |
| | | GAAGCTAGCCTGGATAAGACTCAAATAATAGCGAGAGAGAAACTTGTGAGGGATGTCAGT |
| | | TACTCTTTTCAAGAGTTTCAAGGTATTCAGCGGCAGTTTACCCAAGTAATGAAACAAGTT |
| | | AATGAAAGAGCAAAAGAATCTCTTGAAGCAAGTGAGATGGCAAATGATGCTGCTTTATTA |
| | | GATGAAGAACAAAGGCAGAATAGCTCAAAAAGTACTCGAATACCAGGCAGCCAAATAGTC |
| | | ATTGAGAGAGACCCGATAAATAACGAAGAGTTTGCTTATCAGCAAAATCTTATCGAGCAA |
| | | AGAGACCAGGAAATCAGCAATATTGAAAGAGGTATAACGGAACTGAACGAAGTTTTTAAA |
| | | GATTTGGGAAGTGTTGTTCAACAGCAGGGTGTGCTAGTTGACAATATTGAAGCAAATATT |
| | | TATACAACGTCAGATAACACTCAATTGGCTTCAGACGAGCTAAGGAAGGCCATGCGGTAC |
| | | CAAAAACGTACGAGCAGATGGAGGGTGTATTTGTTGATTGTGCTTCTCGTAATGCTTCTT |
| | | TTTATTTTTCTCATTATGAAATTGTAA |
| YOR124C* | UBP2 | ATGCCGAACGAAGATAATGAACTTCAAAAAGCAATTGAGAACCATCATAATCAACTACTA |
| | | AACCAGGATAAAGAAAATGCTGACAGAAATGGGTCTGTTATAGAAGACCTCCCATTATAC |
| | | GGGACAAGTATAAACCAGCAGTCTACCCCTGGAGATGTTGACGATGGAAAACACTTACTG |
| | | TATCCAGATATTGCCACCAACCTACCACTGAAGACTTCTGACAGACTTTTGGACGATATA |
| | | CTTTGCGATACTATTTTTCTCAATTCTACAGACCCGAAGGTCATGCAAAAGGGCCTGCAA |
| | | TCGAGGGGTATTTTAAAAGAGTCTATGCTTTCTTACTCAACTTTCAGAAGTAGTATTCGC |
| | | CCTAACTGCTTGGGTTCATTAACTGATCAAGTGGTTTTTCAAACAAAATCCGAGTATGAT |
| | | TCCATTTCATGCCCAAAATATAATAAAATACATGTATTTCAGGCGGTCATCTTTAATCCA |
| | | TCACTGGCAGAACAGCAAATTTCAACTTTTGATGATATTGTTAAAATTCCTATTTATCAT |
| | | CTTAAGGTTAGCGTAAAAGTCCGCCAAGAACTGGAGCGGTTGAAGAAGCATGTCGGTGTT |
| | | ACTCAATTCCACTCACTAGATCATTTGCACGAATACGATCGAGTAGACCTTTCGACTTTT |
| | | GATTCTTCCGATCCTAATTTGTTGGATTACGGTATTTACGTTTCTGATGATACTAACAAA |
| | | CTGATCTTGATTGAAATTTTTAAACCCGAGTTTAATTCACCTGAAGAGCATGAGAGTTTT |
| | | ACTGCCGACGCAATTAAGAAGAGATACAATGCTATGTGTGTAAAAAATGAATCACTAGAT |
| | | AAAAGCGAGACGCCATCTCAAGTTGACTGTTTTTACACACTTTTTAAAATTTTTAAAGGG |
| | | CCTTTGACGAGGAAAAGTAAAGCGGAACCTACAAAGACAATTGATTCTGGAAATTTGGCC |
| | | CTTAACACTCACCTGAATCCTGAATGGTTAACGTCCAAGTATGGATTTCAAGCAAGCTCA |
| | | GAAATCGATGAGGAAACTAATGAGATATTTACTGAATACGTCCCTCCAGATATGGTGGAC |
| | | TATGTAAACGATTTGGAGACAAGAAAAATTCGAGAATCGTTTGTGAGGAAGTGTTTACAA |
| | | CTGATATTTTGGGGTCAACTATCTACCTCATTACTGGCACCTAATTCTCCCTTGAAAAAT |
| | | ACGAAAAGCGTAAAGGGAATGTCTTCATTACAAACTTCTTTCTCAACACTACCTTGGTTC |
| | | CATTTATTGGGAGAATCCAGAGCAAGGATTCTATTAAATTCCAATGAGCAAACTCATTCG |
| | | CCTTTGGACGCAGAACCTCATTTTATTAATCTTTCCGTTTCGCATTATTATACCGATAGA |
| | | GATATAATCAGAAACTACGAATCTTTGTCTTCTTTGGATCCTGAAAATATTGGGCTGTAT |
| | | TTTGACGCACTGACATACATTGCAAATAGGAAGGGGGCATATCAATTGATTGCTTACTGT |
| | | GGAAAACAGGACATTATAGGCCAAGAAGCTCTAGAAAATGCTTTGTTAATGTTTAAAATT |
| | | AACCCTAAAGAGTGTAACATCTCCGAATTAAATGAGGCGACTTTGCTATCTATTTACAAA |
| | | TATGAAACATCAAATAAGAGCCAAGTAACCTCTAATCACCTAACAAATTTGAAAAATGCT |
| | | CTAAGATTGTTGGCCAAATATACCAAATCTGACAAACTAAAATTTTACGTCGATCATGAG |
| | | CCCTACAGAGCTTTATCCCAGGCATACGACACACTTTCAATTGACGAGTCTGTTGATGAA |
| | | GACATTATAAAAACTGCATATTCGGTCAAGATTAACGACTCTCCCGGATTAAAGTTGGAT |
| | | TGTGATAGAGCACTTTACACCATTGCTATCAGTAAAAGAAGCCTTGATTTGTTCAATTTT |
| | | TTAACAGAGGAATGCCCACAGTTTTCCAACTATTATGGTCCAGAGAAGCTTGATTACCAA |
| | | GAGGCATTGAAGCTTCTTCAAGTGAATGAAAATGCCTCTGACGAAACCATTTTGAAAATC |
| | | TTTAAACAAAAGTGGTTTGATGAAAACGTTTATGAGCCTGACCAATTTCTTATTTTGAGG |
| | | GCAGCATTGACCAAAATCAGTATAGAAAGAAATTCAACTTTAATCACCAACTTCTTACTA |
| | | ACTGGTACGATAGATCCAAATTCCTTGCCGCCAGAAAATTGGCCAACTGGCATTAATAAT |
| | | ATCGGGAACACCTGTTACCTAAATTCTTTATTACAATATTACTTTTCCATTGCGCCACTA |
| | | AGAAGATATGTATTGGAATATCAAAAAACGGTAGAAAATTTCAATGACCACCTCTCTAAT |
| | | AGTGGGCATATTAGAAGAATTGGTGGAAGAGAAATTAGTAGAGGCGAAGTGGAAAGATCT |
| | | ATTCAATTCATATACCAACTTCGCAACCTTTTCTATGCGATGGTTCATACAAGAGAAAGA |
| | | TGTGTAACACCCTCAAAAGAGCTAGCATATTTGGCATTTGCTCCAAGTAATGTTGAAGTA |
| | | GAATTTGAAGTGGAAGGCAATAAAGTAGTTGATCAAACAGGAGTTCTTTCGGATTCAAAG |
| | | AAGGAAACAACGGATGACGCATTTACTACAAAAATAAAGGATACAAGCCTGATTGATTTA |
| | | GAAATGGAAGATGGCCTTAATGGCGATGTTGGTACAGATGCGAACAGAAAAAAAAATGAA |
| | | TCGAATGATGCTGAAGTAAGTGAGAACGAAGATACAACAGGATTAACTTCACCTACGCGT |
| | | GTGGCAAAAATCAGTTCTGATCAATTAGAAAATGCTTTGGAAATGGGTAGGCAACAAGAT |
| | | GTTACTGAATGCATAGGAAACGTGTTATTTCAGATAGAAAGCGGTTCAGAGCCTATCCGA |
| | | TATGATGAAGACAACGAGCAATATGACTTGGTTAAGCAACTATTTTATGGTACTACTAAA |
| | | CAAAGTATTGTTCCTTTGTCCGCAACAAATAAAGTCCGTACGAAAGTTGAAAGATTCCTA |
| | | TCGTTACTGATAAATATTGGCGATCATCCTAAAGATATTTATGATGCGTTTGATTCTTAT |
| | | TTTAAAGACGAATATCTGACAATGGAAGAGTATGGTGATGTTATACGTACCGTTGCTGTT |
| | | ACAACTTTTCCTACTATTTTGCAGGTACAAATCCAAAGAGTTTATTACGATCGTGAAAGA |
| | | TTAATGCCGTTTAAATCCATTGAGCCCTTACCATTCAAAGAAGTTATTTACATGGACAGA |
| | | TACGCGGATACAGAGAACCCTTTATTGTTGGCAAAAAAGAAAGAAACAGAAGAAATGAAG |
| | | CAAAAGTTGAAGGTAATGAAAAATAGACAAAGAGAGCTTTTGAGTCGTGATGATTCAGGG |
| | | CTTACAAGGAAGGATGCATTTTTGGAGAGTATCAAGCTATTGGAATCGGATACCATAAAG |
| | | AAAACTCCTTTAAAAATTGAGGCTGCTAATGATGTGATAAAGACGCTGAGAAACAACGTT |
| | | CAAAATATCGATAATGAATTGATGAAATTATACAATGATATCAACAGTTTGGAAGAGAAA |
| | | ATAAGCCATCAATTTGACGATTTCAAGGAATATGGTTACTCACTGTTTTCGGTTTTTATT |
| | | CATCGCGGCGAGGCCAGTTATGGTCACTATTGGATATATATCAAGGACAGAAATCGCAAT |
| | | GGAATTTGGAGGAAGTACAATGATGAAACCATCAGTGAGGTCCAGGAAGAGGAGGTCTTC |
| | | AATTTCAATGAGGGTAACACTGCAACTCCATATTTCCTAGTATATGTCAAACAAGGACAA |
| | | GAAGGTGATATTGAGCCATTGAAAAGAATTCTAAAGTAG |
| YOR152C* | | ATGTTTAATTTAATTTTATGGCCCTTATTTTTGCTTACCTCAGTTGCCATTCCTTTGCAG |
| | | CTGACATTAGAAGTGGTCTATCTGACGTCTTCCGTTGACTTCTCAAAGGCAAGTGCTGCT |
| | | AAAACCGCTACTTCTTTGGGTCAGTCCCCAGTTGTCATCACCATTTATAAATCACTGTTA |
| | | AAATATTGGAGTCTTTACGAGTTCATACATTTTATTTACCTCTATACTCCTATTGATGCC |
| | | TTTCTGAATTTCTTACCATTTACCTCTTTACTTATGTCGTTTGGAAGTATTTGCTTGACA |
| | | AGAGAGCTGGTATATGACTTCATTGCCTTTATGGAATCTCAAAACAAGCTTACCGGATTC |
| | | CTTAATAAGATAACAGAACCGAATTTTAACAGTTATTTACTGTTTTCTTCAATTTATAAC |
| | | ATTTGGTTTGCAGATGACACCAATGACAAATTCTTATTCGGTAAATTAACTCAGATTTTA |
| | | ATATCTGTAACAAAAAGGTACGAATTTCCAAGAACATTCTACTTAGCAAAAGTTTCTGAT |
| | | TTCCTACAAAATCTCATTCTAACGAGATTGAGACCCTTTGTAACGGAGCAACCCCAAGGC |
| | | GACAAAAATCGCTATCAAAACGGTGACCGTGAATCTACAAAGAATGGAGCTGCTTATCAG |
| | | AAGTCTTCACAACAATCATCGTCTTTCGAACAAAATTTTACCAGCACTGAGTTCCCGAAT |
| | | GATTATGATTTTATGGAGGATATTCTAGATGAGACAACTGAATTGGATTAG |
| YOR290C* | SNF2 | ATGAACATACCACAGCGTCAATTTAGCAACGAAGAGGTCAACCGCTGCTATTTAAGATGG |
| | | CAGCATTTGAGAAATGAACACGGAATGAACGCTCCGAGTGTGCCGGAATTTATCTATTTG |
| | | ACTAAAGTGTTGCAATTTGCTGCCAAGCAGCGACAAGAATTACAAATGCAACGCCAACAG |
| | | CAAGGCATTTCAGGTTCTCAGCAAAATATTGTTCCAAATTCTTCAGATCAAGCAGAACTG |
| | | CCGAATAATGCTTCCTCGCACATTTCTGCTTCTGCTTCCCCGCATTTAGCTCCGAATATG |
| | | CAATTGAATGGAAATGAGACCTTTAGCACTTCTGCTCATCAATCACCAATCATGCAAACA |
| | | CAGATGCCATTAAATAGCAATGGTGGTAATAACATGCTACCACAACGGCAATCTTCGGTT |
| | | GGTTCATTAAATGCAACTAATTTTTCACCAACACCAGCAAACAATGGAGAAAATGCTGCG |
| | | GAGAAACCAGATAACTCAAACCATAATAACTTAAACCTTAATAATTCCGAGCTTCAACCG |
| | | CAAAATCGATCATTACAAGAACACAACATACAGGATTCAAATGTGATGCCAGGTTCGCAG |
| | | ATAAATTCTCCTATGCCCCAGCAGGCGCAGATGCAGCAGGCACAATTCCAAGCACAACAG |
| | | GCGCAACAAGCGCAACAGGCACAACAGGCACAACAAGCACAAGCGAGACTGCAACAGGGG |
| | | AGAAGATTACCCATGACAATGTTTACTGCCGAGCAATCCGAACTGTTAAAGGCCCAAATC |
| | | ACATCTCTAAAATGTCTAGTAAATAGAAAGCCTATTCCGTTTGAATTTCAGGCTGTTATC |
| | | CAAAAATCTATTAACCATCCTCCAGATTTCAAAAGAATGTTACTGTCTTTGAGTGAGTTT |
| | | GCGAGAAGAAGACAACCCACAGATCAAAATAACCAATCAAATCTCAATGGCGGCAACAAT |
| | | ACGCAGCAACCCGGCACCAATTCCCACTACAATAACACTAACACTGACAACGTTTCTGGG |
| | | CTTACCAGAAATGCACCTTTAGATAGCAAAGACGAAAATTTTGCATCTGTTTCACCAGCT |
| | | GGTCCATCTAGTGTACATAATGCGAAGAATGGCACCCTCGATAAAAATTCGCAAACTGTA |
| | | AGTGGCACTCCAATCACGCAAACAGAATCGAAAAAAGAGGAAAACGAAACCATTTCAAAC |
| | | GTAGCTAAAACAGCTCCTAATAGTAATAAGACCCACACCGAGCAAAATAACCCGCCAAAA |
| | | CCGCAGAAACCCGTTCCTTTAAACGTCCTTCAAGATCAATATAAGGAAGGAATAAAAGTG |
| | | GTAGATATAGATGATCCTGATATGATGGTTGATTCTTTTACGATGCCCAATATCTCACAT |
| | | AGTAATATCGATTATCAGACTTTATTGGCCAATTCGGATCATGCAAAGTTTACTATTGAA |
| | | CCTGGCGTTCTACCGGTTGGTATTGACACACATACCGCTACTGACATTTACCAAACTTTA |
| | | ATTGCATTGAACCTAGATACTACGGTAAATGACTGTTTGGATAAGCTACTAAATGACGAA |
| | | TGTACGGAATCAACCAGGGAAAATGCTCTTTACGATTACTACGCCTTACAATTATTACCG |
| | | TTACAAAAAGCCGTAAGAGGTCATGTATTACAATTTGAATGGCATCAAAATTCTTTATTA |
| | | ACCAATACACATCCAAATTTTCTTTCGAAAATAAGAAACATCAACGTTCAAGATGCATTG |
| | | CTAACAAACCAACTTTATAAGAATCATGAATTACTTAAGTTAGAAAGGAAGAAGACTGAG |
| | | GCAGTGGCCAGATTAAAGTCGATGAATAAATCCGCCATCAATCAATATAATAGAAGACAA |
| | | GATAAAAAGAATAAAAGGTTGAAGTTTGGCCATAGACTCATTGCTACGCATACTAATTTA |
| | | GAAAGGGATGAGCAAAAGAGAGCTGAAAAAAAGGCAAAAGAACGTTTACAGGCTTTAAAG |
| | | GCGAACGATGAGGAAGCCTATATAAAATTATTAGACCAAACCAAAGATACAAGAATTACA |
| | | CATCTTCTAAGGCAAACAAACGCATTTTTAGATTCTTTAACAAGAGCCGTGAAAGATCAA |
| | | CAAAAGTACACTAAAGAAATGATTGATTCCCACATAAAGGAAGCATCGGAGGAAGTGGAT |
| | | GATTTGTCCATGGTCCCCAAGATGAAGGATGAAGAATATGATGACGACGACGACAATTCT |
| | | AACGTCGATTATTACAATGTCGCACATAGAATTAAAGAAGATATCAAGAAGCAGCCTTCA |
| | | ATATTGGTTGGTGGTACCTTAAAGGACTATCAAATCAAGGGTTTACAGTGGATGGTTTCG |
| | | CTTTTCAATAATCATTTAAATGGTATTTTAGCAGACGAAATGGGTCTTGGTAAGACTATC |
| | | CAGACGATATCCTTACTTACATATTTATATGAAATGAAGAATATTCGTGGGCCATATTTA |
| | | GTCATTGTTCCTTTATCTACACTGTCAAATTGGAGCAGTGAATTCGCGAAATGGGCTCCT |
| | | ACCTTAAGAACTATATCATTTAAAGGTTCGCCAAATGAGAGGAAAGCCAAACAGGCTAAA |
| | | ATAAGGGCAGGTGAATTTGATGTAGTTCTGACTACCTTTGAGTATATTATTAAAGAAAGA |
| | | GCACTTTTATCCAAGGTCAAGTGGGTCCATATGATTATTGATGAAGGCCATAGAATGAAA |
| | | AATGCGCAATCCAAGCTTTCTTTAACCTTGAACACACATTACCATGCAGATTATAGATTA |
| | | ATTTTGACAGGTACACCACTTCAAAACAACTTGCCAGAATTATGGGCCTTATTGAATTTT |
| | | GTGTTACCCAAGATTTTCAATTCCGTGAAATCTTTTGATGAATGGTTCAATACACCCTTT |
| | | GCCAACACCGGTGGGCAAGATAAAATTGAGTTGAGTGAAGAAGAGACGCTTTTGGTTATC |
| | | AGGAGATTGCATAAAGTCTTGAGACCCTTTTTATTGCGTCGTTTGAAAAAGGATGTAGAA |
| | | AAAGAATTACCGGATAAAGTTGAGAAGGTAGTGAAGTGCAAAATGAGCGCTTTACAACAA |
| | | ATTATGTACCAACAAATGTTGAAATATCGTCGTCTTTTTATCGGTGATCAAAATAATAAG |
| | | AAAATGGTAGGTTTAAGAGGTTTCAATAATCAAATCATGCAACTAAAGAAAATCTGTAAT |
| | | CATCCTTTTGTATTTGAAGAAGTTGAGGACCAAATCAATCCAACAAGAGAAACGAATGAT |
| | | GATATTTGGCGTGTTGCTGGTAAATTTGAACTATTAGATAGAATTTTGCCTAAGCTTAAG |
| | | GCAACTGGGCATAGAGTATTAATTTTTTTTCAAATGACCCAAATTATGGATATTATGGAA |
| | | GATTTTTTAAGGTACATTAATATCAAGTACCTGAGGTTAGATGGTCATACAAAATCTGAT |
| | | GAACGTAGTGAGTTGTTACGTCTATTTAACGCCCCTGATTCTGAATACTTGTGCTTTATC |
| | | TTATCAACTAGAGCGGGTGGTTTGGGTTTAAATTTACAAACTGCAGACACTGTCATTATT |
| | | TTTGATACTGACTGGAATCCTCATCAAGATTTGCAAGCTCAGGATAGAGCGCATAGAATA |
| | | GGTCAGAAGAATGAAGTTAGAATTCTAAGATTGATTACTACGAATTCAGTTGAAGAAGTT |
| | | ATCCTAGAGAGAGCCTACAAGAAGCTAGATATCGATGGTAAAGTCATTCAAGCAGGTAAA |
| | | TTCGATAACAAATCTACATCGGAGGAACAAGAAGCGTTGCTTAGATCTTTGCTGGATGCA |
| | | GAAGAGGAACGTAGAAAGAAAAGAGAGTCTGGTGTTGAAGAAGAAGAGGAGTTAAAAGAC |
| | | TCAGAAATCAACGAAATTCTTGCTCGAAATGACGAAGAAATGGCTGTTCTTACAAGGATG |
| | | GATGAAGATAGATCAAAGAAGGAGGAAGAATTGGGAGTAAAGAGCCGTCTCTTAGAAAAG |
| | | AGTGAATTACCTGATATTTACAGTAGAGATATTGGTGCCGAGCTCAAACGTGAAGAATCA |
| | | GAATCTGCTGCTGTTTACAATGGAAGGGGTGCAAGAGAACGTAAGACGGCAACTTACAAC |
| | | GATAATATGTCTGAAGAACAGTGGTTAAGACAATTTGAAGTTAGCGATGATGAAAAAAAT |
| | | GACAAGCAGGCAAGAAAACAACGAACAAAGAAGGAAGATAAGAGCGAGGCGATTGATGGG |
| | | AATGGAGAAATTAAAGGCGAAAACATTGACGCTGATAATGACGGTCCTAGAATTAATAAT |
| | | ATATCGGCCGAAGATCGGGCAGATACGGATTTGGCAATGAATGACGATGATTTCTTGTCT |
| | | AAAAAGAGAAAGGCAGGAAGGCCCCGTGGAAGACCTAAGAAAGTGAAACTTGAAGGATCT |
| | | GAAAATTCTGAACCACCAGCGCTTGAATCAAGTCCAGTTACTGGCGATAACTCTCCCAGT |
| | | GAGGATTTTATGGACATTCCAAAACCACGTACAGCGGGAAAAACATCTGTTAAATCAGCA |
| | | AGAACTTCAACAAGGGGCCGTGGTCGCGGTCGTGGTCGTGGTCGAGGGCGAGGACGTGGA |
| | | CGAGGAAGGCCGCCAAAAGCAAGAAATGGGCTTGATTATGTCCGCACGCCCGCTGCGGCA |
| | | ACGAGTCCTATTGATATTAGAGAGAAAGTTGCTAAACAAGCTCTAGACTTATACCACTTT |
| | | GCTCTCAATTATGAAAATGAAGCTGGAAGGAAACTTTCTGACATTTTCCTCTCCAAACCT |
| | | TCCAAAGCACTATATCCCGACTACTATATGATTATTAAGTACCCTGTCGCCTTTGATAAT |
| | | ATTAACACGCATATTGAAACGCTAGCTTACAACTCCCTTAAAGAGACCCTTCAAGACTTT |
| | | CATCTGATATTTTCCAATGCTAGGATATACAATACTGAGGGTTCAGTAGTATACGAAGAC |
| | | TCTTTAGAACTGGAAAAAGTGGTTACGAAGAAATACTGTGAGATAATGGGCGATAACTCA |
| | | CAACTCGACTTTACTGAATTTGATGAACAGTATGGCACCAGACCCCTCGTCTTGCCTCCT |
| | | GTTGTTACTTCAAGCGTGGCTGAATCTTTCACAGATGAAGCGGACTCGAGCATGACAGAA |
| | | GCGAGTGTATAG |
| YOR298C-A* | MBF1 | ATGTCTGACTGGGATACAAATACTATTATTGGTAGCAGAGCAAGAGCTGGTGGTTCTGGC |
| | | CCAAGGGCCAACGTTGCCAGATCTCAAGGCCAAATTAATGCTGCAAGAAGACAGGGGTTG |
| | | GTTGTTTCCGTTGACAAGAAGTACGGATCAACCAATACGAGGGGTGACAACGAGGGTCAG |
| | | AGGTTGACCAAGGTCGACCGTGAGACAGATATTGTCAAACCCAAGAAATTGGATCCAAAT |
| | | GTCGGTAGGGCCATCTCGCGTGCCAGAACAGACAAGAAAATGTCGCAGAAGGATTTGGCA |
| | | ACCAAAATTAACGAAAAGCCAACCGTGGTAAACGATTATGAAGCCGCTAGAGCTATTCCA |
| | | AACCAACAAGTTTTGTCTAAGTTGGAAAGAGCCTTAGGCGTTAAATTGAGAGGTAACAAC |
| | | ATCGGTTCGCCTTTGGGAGCTCCAAAGAAGAAATGA |
| YOR302W | | ATGTTTAGCTTATCGAACTCTCAATACACCTGCCAAGACTACATATCTGACCACATCTGG |
| | | AAAACTAGCTCCCACTAA |
| YML013C-A* | | ATGCTATTTTATCATTGTTCTTCATTTTCCTCATCTTCTTCATCTTCTTCATCCAGTGCT |
| | | TCCACTAGAAGACTGCCATTTGGCCATAGCTGGGGCACTTGGTCCACCGACATGTGCTCG |
| | | TTAGGATGCACAGTATACTTAGGGAAGGGCGATACCAATTCAAAGTCGAATTCAAGTTTA |
| | | CCACGTTCGAGATCAAATGAGGTAAGCTCGTTGTTGATGATGTGGCCGAGCTCTTCGATA |
| | | GTGGTAGCAGTAGCAGTTTCGAGTTGTCCTTCTTTAAAGCAGAGCATATCGTCGTCTGCA |
| | | GATAATTGCCTGATTTTGTCCTGGAGGGCGTTAGACCATTCTGCGGGGTCACTAGAATAT |
| | | ACCGCGAGGTATATGTGA |

1. A transgenic plant which plant displays altered expression of a gene selected from the group consisting of:
(a) decreased expression of a gene which confers sensitivity to fungal disease or mycotoxin, wherein said sensitivity gene is selected from the group consisting of: YGL139W, YDL054C, YDR157W, YDL056W, YGL135W, YKR010C, YLR099C, YLR193C, YGL110C, YKR012C, YDL045W-A, YLR262C-A, YDR533C, YPR047W, YMR187C, YNL011C, YKR046C, YER017C, YLR260W, YGR241C, YDL044C, YMR310C, YOL101C, YPR100W, YHR038W, YDR519W, YPL219W, YJL119C, YOL095C, YGL064C, YFR019W, YDR529C, YDR065W, YGR283C, YJL152W, YGL119W, YHR051W, YMR064W, YDL173W, YBR268W, YHR091C, YDR296W, YDR519W, YKR006C, YBR122C, YBR179C, YGR101W, YBR126C, YLR312W-A, YKL003C, YLR270W, YLR199C, YFR032C-A, YPL271W, YPL104W, YDR509W, YNL284C, YNL005C, YDR377W, YMR267W, YPR116W, YCR003W, YCR071C, YOL009C, YER058W, YGR219W, YCR046C, YDR462W, YPL052W, YJR101W, YPR166C, YER061C, YGL218W, YBR251W, YBR282W, YJL102W, YGL064C, YNL073W, YOR201C, YPL118W, YLL033W, YMR287C, YML110C, YLR439W, YDL044C, YNL081C, YER050C, YKL134C, YER087W, YJL193W, YLL006W, YDL056W, YBL090W, YOR241W, YLR295C, YBR120C, YCR024C, YMR293C, YBR163W, YPL173W, YPL005W, YBR104W, YER122C, YGL246C, YLR139C, YGR215W, YJL132W, YHR147C, YAL010C, YPL057C, YOL023W, YBR162W-A, YOR205C, YDL067C, YDL050C, YJL189W, YCR028C-A, YGR220C, YBR101C, YJL208C, YBR144C, and YGL251C; and
(b) increased expression of a gene which confers resistance to a fungal disease or mycotoxin wherein said resistance gene is selected from the group consisting of: YAL002W, YAL013W, YAL021C, YAL047C, YBL058W, YBL071W-A, YBL093C, YBR023C, YBR081C, YBR112C, YBR267W, YBR279W, YBR289W, YCR020W-B, YCR044C, YCR094W, YDL013W, YDL023C, YDL075W, YDL106C, YDR049W, YDR264C, YDR266C, YDR433W, YDR442W, YDR495C, YDR532C, YEL044W, YER070W, YER087W, YER151C, YFR010W, YGL012W, YGL038C, YPL050C, YPL084W, YPL129W, YPL254W, YPL268W, YGL058W, YGL070C, YGR006W, YGR063C, YGR092W, YGR104C, YGR196C, YGR262C, YGR271W, YHL025W, YHR013C, YHR030C, YJL028W, YJL095W, YJL129C, YJL175W, YJL184W, YJR090C, YJR102C, YKL002W, YKL139W, YKL213C, YKR023W, YKR082W, YKR093W, YLR025W, YLR027C, YLR226W, YLR234W, YLR240W, YLR322W, YLR330W, YLR337C, YLR338W, YPR072W, YPR173C, YPL042C, YPL045W, YKR004C-A, YLR370C, YLR417W, YML008C, YML062C, YML094W, YML112W, YMR032W, YMR077C, YMR202W, YMR263W, YNL054W, YNL064C, YNL084C, YNL097C, YNL133C, YNL140C, YNL147W, YNL229C, YNL236W, YNR051C, YNR052C, YOL004W, YOL051W, YOL068C, YOL086C, YOL148C, YOR026W, YOR035C, YOR036W, YOR124C, YOR152C, YOR290C, YOR298C-A, YOR302W, and YML013C-A;
wherein said altered expression confers said plant with increased resistance to a fungal disease or mycotoxin as compared to a control plant without said altered expression.

2. The transgenic plant of claim 1, wherein said plant displays increased expression of a resistance gene.

3. The transgenic plant of claim 1, wherein said plant displays decreased expression of a sensitivity gene.

4. The transgenic plant of claim 1, wherein said mycotoxin is a trichothecene.

5. The transgenic plant of claim 1, wherein said trichothecene is selected from the group consisting of trichodermin, deoxynivalenol, diacetoxyscirpeol, zearalenone, nivalenol, 3-acetyldeoxynivalenol, 15-acetyldeoxynivalenol, fusarenon-X, T-2 toxin, and trichothecin (T-cin).

6. The transgenic plant of claim 1, which is a monocot plant.

7. The transgenic plant of claim 1, which is a cereal crop plant.

8. The transgenic plant of claim 7, wherein the cereal crop plant is maize, rice, wheat, oat, barley or wheat.

9. The transgenic plant of claim 1, which is a dicot plant.

10. A non-naturally occurring seed derived from the transgenic plant of claim 1.

11. A method of making a transgenic plant having increased resistance to fungal disease or mycotoxin, comprising preparing a transgenic plant having a genome that contains an exogenous nucleic acid comprising a transgene encoding a gene selected from the group consisting of YAL002W, YAL013W, YAL021C, YAL047C, YBL058W, YBL071W-A, YBL093C, YBR023C, YBR081C, YBR112C, YBR267W, YBR279W, YBR289W, YCR020W-B, YCR044C, YCR094W, YDL013W, YDL023C, YDL075W, YDL106C, YDR049W, YDR264C, YDR266C, YDR433W, YDR442W, YDR495C, YDR532C, YEL044W, YER070W, YER087W, YER151C, YFR010W, YGL012W, YGL038C, YPL050C, YPL084W, YPL129W, YPL254W, YPL268W, YGL058W, YGL070C, YGR006W, YGR063C, YGR092W, YGR104C, YGR196C, YGR262C, YGR271W, YHL025W, YHR013C, YHR030C, YJL028W, YJL095W, YJL129C, YJL175W, YJL184W, YJR090C, YJR102C, YKL002W, YKL139W, YKL213C, YKR023W, YKR082W, YKR093W, YLR025W, YLR027C, YLR226W, YLR234W, YLR240W, YLR322W, YLR330W, YLR337C, YLR338W, YPR072W, YPR173C, YPL042C, YPL045W, YKR004C-A, YLR370C, YLR417W, YML008C, YML062C, YML094W, YML112W, YMR032W, YMR077C, YMR202W, YMR263W, YNL054W, YNL064C, YNL084C, YNL097C, YNL133C, YNL140C, YNL147W, YNL229C, YNL236W, YNR051C, YNR052C, YOL004W, YOL051W, YOL068C, YOL086C, YOL148C, YOR026W, YOR035C, YOR036W, YOR124C, YOR152C, YOR290C, YOR298C-A, YOR302W, and YML013C-A; wherein expression of the transgene in the transgenic plant confers increased resistance to fungal disease or mycotoxin to a non-transgenic control plant.

12. The method of claim 11, wherein said preparing a transgenic plant comprises transforming a protoplast from a cell of a plant with the exogenous nucleic acid to produce a transformed protoplast, and generating the transgenic plant from the transformed protoplast.

13. The method of claim 11, wherein said preparing a transgenic plant comprises introducing the exogenous nucleic acid into tissue of a plant to produce transformed plant tissue, and regenerating the transgenic plant from the transformed plant tissue.

14. A method of making a transgenic plant having increased resistance to fungal disease or mycotoxin, comprising preparing a transgenic plant having a genome that contains a disruption in the gene selected from the group consisting of: YGL139W, YDL054C, YDR157W, YDL056W, YGL135W, YKR010C, YLR099C, YLR193C, YGL110C, YKR012C, YDL045W-A, YLR262C-A, YDR533C, YPR047W, YMR187C, YNL011C, YKR046C, YER017C, YLR260W, YGR241C, YDL044C, YMR310C, YOL101C, YPR100W, YHR038W, YDR519W, YPL219W, YJL119C, YOL095C, YGL064C, YFR019W, YDR529C, YDR065W, YGR283C, YJL152W, YGL119W, YHR051W, YMR064W, YDL173W, YBR268W, YHR091C, YDR296W, YDR519W, YKR006C, YBR122C, YBR179C, YGR101W, YBR126C, YLR312W-A, YKL003C, YLR270W, YLR199C, YFR032C-A, YPL271W, YPL104W, YDR509W, YNL284C, YNL005C, YDR377W, YMR267W, YPR116W, YCR003W, YCR071C, YOL009C, YER058W, YGR219W, YCR046C, YDR462W, YPL052W, YJR101W, YPR166C, YER061C, YGL218W, YBR251W, YBR282W, YJL102W, YGL064C, YNL073W, YOR201C, YPL118W, YLL033W, YMR287C, YML110C, YLR439W, YDL044C, YNL081C, YER050C, YKL134C, YER087W, YJL193W, YLL006W, YDL056W, YBL090W, YOR241W, YLR295C, YBR120C, YCR024C, YMR293C, YBR163W, YPL173W, YPL005W, YBR104W, YER122C, YGL246C, YLR139C, YGR215W, YJL132W, YHR147C, YAL010C, YPL057C, YOL023W, YBR162W-A, YOR205C, YDL067C, YDL050C, YJL189W, YCR028C-A, YGR220C, YBR101C, YJL208C, YBR144C, and YGL251C, wherein said disruption results in decreased expression of said gene.

15. The method of claim 14, wherein said preparing a transgenic plant comprises transforming a protoplast from a cell of a plant with the disrupted gene to produce a transformed protoplast, and generating the transgenic plant from the transformed protoplast.

16. The method of claim 11, wherein said preparing a transgenic plant comprises introducing the disrupted gene into tissue of a plant to produce transformed plant tissue, and regenerating the transgenic plant from the transformed plant tissue.
